(19) United States
(12) Patent Application Publication
(10) Pub. No.: US 2004/0254182 A1
(43) Pub. Date: Dec. 16, 2004

(54) Enhanced propertied pharmaceuticals

(76) Inventors: Mark Joseph Mulvihill, East Northport, NY (US); Steven Howard Shaber, Indianapolis, IN (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/182,076

(22) PCT Filed: Jan. 26, 2001
(86) PCT No.: PCT/US01/00653

Related U.S. Application Data
(63) Continuation-in-part of application No. 09/493,865, filed on Jan. 28, 2000, now Pat. No. 6,376,548.
(60) Provisional application No. 60/178,878, filed on Jan. 28, 2000.

(51) Int. Cl. A61K 31/675; A61K 31/541; A61K 31/5377; A61K 31/496

(52) U.S. Cl. 514/227.8; 514/235.5; 514/252.13; 514/255.02; 514/253.08

(57) ABSTRACT

This invention relates to compounds which are useful as enhanced propertied pharmaceutical compounds for both human and veterinary application. The pharmaceutical compounds which are suitable for use in this invention are those compounds which can be substituted with a moiety, said moiety comprising a substituent which enhances or changes the properties of the pharmaceutical compound. The chemical modification of drugs into labile derivatives with enhanced physicochemical properties that enable better transport through biological barriers is a useful approach for improving-drug delivery.

This invention relates to compounds which are useful as enhanced propertied pharmaceutical compounds for both human and veterinary application. The pharmaceutical compounds which are suitable for use in this invention are those compounds which can be substituted with a moiety, said moiety comprising a substituent which enhances or changes the properties of the pharmaceutical compound. The chemical modification of drugs into labile derivatives with enhanced physicochemical properties that enable better transport through biological barriers is a useful approach for improving-drug delivery. This modification can be conveniently practiced on ionizable molecules containing moieties such as a carboxy group, an amino group, a hydroxy group, a mercapto group or a group containing an appropriately substituted phosphorous atom that can be utilized for derivatization in order to modify their ionization at physiological pH and to render desirable partition and solubility properties. A necessary requirement of this approach is that the enhanced propertied drug is non-toxic and, when administered to a warm-blooded animal including a human being, is enzymatically and/or chemically cleaved in such a manner as to release the drug at its target or site of activity, quantitatively and at a desirable rate, while the remaining cleaved moiety remains non-toxic and is metabolized in such a manner that non-toxic metabolic products are produced. It is naturally also desirable that the enhanced propertied drug can be provided without excessive costs in connection with its production, in particular without an appreciable loss of the unmodified drug itself during its production and recovery, since the unmodified drug is usually the more expensive part of the enhanced propertied drug.

Furthermore, the new substituent on the original pharmaceutical compound may itself optionally comprise a pharmaceutical compound which may be the same as or different from the original pharmaceutical compound. This allows a combination of pharmaceutical compounds to be applied simultaneously as a single compound to the host. The application of such a compound provides many advantages such as a greater spectrum of activity against the disease being treated and an attenuation of the build up of disease resistance since the disease is being controlled with two different modes of action. This type of enhanced propertied pharmaceutical compound will naturally comprise two different pharmaceutical moieties which are compatible with one another and which can be used without an antagonistic interactive effect upon the host. Such combinations should be apparent to one of ordinary skill in the art.

U.S. Pat. No. 4,760,057, U.S. Pat. No. 4,916,230, U.S. Pat. No. 5,401,868, U.S. Pat. No. 5,459,155, U.S. Pat. No. 5,583,148, U.S. Pat. No. 5,684,018, WO 98/16537, WO 98/43970 and WO 99/61017 describe certain pharmaceutical compounds which are substituted with moieties to alter the properties of pharmaceutical compounds; however, the moieties employed in the compounds of the present invention are neither disclosed nor suggested.

U.S. Pat. No. 5,284,863, U.S. Pat. No. 4,912,090, U.S. Pat. No. 5,385,880, U.S. Pat. No. 5,391,537, JP 1275565 A2, WO 96/36613, WO 99/35141, WO 00/29378, WO 00/40582, DE 4343831 A1, JP 53-43571 B and JP 54-1771 B disclose certain pesticidal compounds which are substituted with moieties to alter the properties of pesticidal compounds; however, the moieties employed in the compounds of the present invention are neither disclosed nor suggested.

The pharmaceutical compounds which may be advantageously employed to produce the enhanced propertied human health pharmaceutical compounds of this invention include, but are not limited to, 1-cyclopropyl-6-fluoro-1,4-dihydro-4-oxo-7-(1-piperezinyl)-3-quinolinecarboxylic acid, 1-ethyl-6-fluoro-1,4-dihydro-4-oxo-7-(1-piperazinyl)-1,8-naphthyridine-3-carboxylic acid, accolate, acebutolol, acetaminophen, acetazolamide, acrivistine, acyclovir, adamantamine, adapalene, adenosine phosphate, adrenelone, albendazole, albuterol, albutoin, alendronate sodium, aletamine, aliconazole, alinidine, alisobumal, alizapride, allopurinol, alprazolam, alprenolol, amethopterine, amidephrine, aminorex, amiodarone, amitriptyline, amlodipine, amoxapine, amoxicillin, amphetamine, amrinone, apraclonidine, aprinocid, arthrocine, arthrotec, aspartame, astemizole, atenolol, atorvastatin, atropine, azatadine, azathioprine, azithromycin, aztreonam, baclofen, bamethan, becliconazole, beclomet, benazepril, benzatropine, benzonatate, benzphetamine, benztropine, beperiden, betahistine, bicalutamide, bisacodyl, botalol hydrochloride, brimonidine tartrate, brolaconazole, bromocriptine, bromopheniramine, bucindolol, budesonide, bufenox, bunolol, bupivacaine, buprenorphine, bupropion, buspirone, butaconazole, butopamine, butorphanol, butoxamine, caffeine, cafiminol, cambendazole, captopril, carbamazepam, carbinoxamine, carbuterol, carisoprodal, cartelolol, carvedilol, cefachlor, cefadroxil, cefamandole, cefazolin, cefixime, cefinetazole, cefonicid, cefoperazone, cefotaxime, cefotetan, cefpodoxime, cefprozil, ceftriaxone, celecoxib, centerdrine, cephapirin, cetirizine, chlorambucil, chloramiphene, chlorazepate, chlordiazepoxide, chloroprocaine, chloroquine, chloroxazone, chlorpheniramine, chlorpromazine, chlortermine, chlorthiazide, cimetidine, cinnarizine, ciprofloxacin, cisapride, cisconazole, citalopram, cladribine, clarithromycin, clemastine, clindamycin, clobesol, clofazimine, clomiphene, clonazepam, clonidine, clopidrogrel, clorprenaline, clotrimazole, cloxacillin, clozapine, cocaine, codeine, colchicine, colterol, combivir, cortisine, croconazole, cromolyn sodium, cyclobendazole, cyclobenzaprine, cyclopentolate, cyclophosphamide, cyclopsporin, cyproheptadine, dacarbazine, dactinomycin, dantrolene, deacetylketoconazole, demeclocycline, demethylamitriptyline, demethylimipramine, democonazole, deprenil, desipramine, deterenol, dexpropranolol, diacetolol, diazepam, diazoxide, dibucaine, diclofenac sodium, dicodid, dicyclomine, didanosine, diethylproprion, diflunisal, dihydroergotamine, dilantin, diltiazem, dimenhydrinate, dimethoxyphenethylmine, diphenhydramine, diphenidol, diphenoxylate hydrochloride, dipyridamole, disopyramide, dobutamine, doconazole, donezapil hydrochloride, dopamine, dotycin, doxapram, doxazosin, doxepin, doxylamine, dypyridamole, eberconazole, econazole, EDTA, efavirenz, enalapril, enoxacin, enviroxime, epinephrine, ergotamine, erythromycin, estazolam, ethionamide, etintidine, etodolac, etryptamine, exaprolol, exprenolol, famciclovir, famotidine, felodipine, fenbendazole, fenfluramine, fenmetazole, fenoterol, fentanyl, fenticonazole, fenyripol, fexofenadine, finasteride, flavoxate, fluazepam, flubendazole, fluconazole, fludarabine, fludorex, flufenazin, fluoxetine hydrochloride, fluphenazine, flurazepam, flurbiprofen, fluticasone propionate, fluvastatin, fluvoxamine maleate, folic acid, fosphenyloin, furazolidone, furosemide, gabapentin, ganciclovir, gemfibrozil, genaconazole, gentian violet, glimepiride, glipizide, glyburide, granisetron, guaifenesin, guanethidine, guanfacine hydrochloride, halazepam, haloperidol, homatropine, hydrochlorothiazide, hydrocodone, hydromorphone, hydroxychloroquine, hydroxyitraconazole, hydroxyzine, hyoscyamine, ibuprofen, imipramine hydrochloride, indapamide, indinivar sulfate, indomethacin, iodoquinol, ipratropium bromide, irbesartan, isoconazole, isoniazid, isosornide monnitrate, isotretinoin, itraconazole, ketoconazole, ketoprofen, ketorolac, ketorolac, labetalol, labotolol, lamivudine, lamotrigin, lamotrigine, lansoprazole, levobunolol, levocabastine, levofloxacin, levomethadyl, levorphanol, lidocaine, lincomycin, lisinopril, lomefloxacin, loperidine hydrochloride, loratadine, lorazepam, losartan, lotrisone, lovastatin, loxapine, L-thyroxine, mazindol, mebendazole, mechlorethamine, meclizine hydrochloride, medroxyprogesteron, megestrol, melphalan, meperidine, mepivacaine, mercaptopurine, mesalamine, mesoridazine, metaproterenol, metazoline methadone, methdilazine, methenamine, methimazol, methocarbamol, methochlopramide, methotrexate, methotrimeprazine, methyl dopa, methylphenidate, methysergide, metoclopramide, metolol, metolprolol tartrate, metoprolol, metoprolol succinate, metronidazole, miconazole, midazolam, miloride, minocycline, minoxidil, mitazapine, molindone, mometasone furoate, monopril, monozid, montelukast, morphine, mycophenolate mofetil hydrochloride, N-[3(R)-[2-(piperidin-4-yl)ethyl]-2-piperidon-1-yl]acetyl-3(R)-methyl-beta-alanine, nadolol, nafazodone, naftifine, nalbuphine, nalidixic acid, nalmefene, naloxone, naphazoline, naproxen, natrexone, nedocromil, nefazodone, nelfinavir mesylate, neticonazole, niacin, nicardipine, nicotine, nifedinpine, nifurantin, nimodipine, nitrofurantoin, nitrofurazone, nizatidine, nocodazole, nore phrine, norfloxacina oaan olopatadine hydrochloride, omeprazole, omoconazole, ondansetron, orconazole, orphenadrine, oxaprozin, oxazepam, oxfendazole, obendazole, oxibendazole, oxiconazole, oxmetidine, oxybutynin, oxycodone, oxymetazoline, oxymorphone, oxytetracycline, pamatolol, papaverine, parbendazole, parconazole, paroxetine, pemoline, penbutalol, penicillin VK, pentazocine, pentostatin, pentoxifylline, perphenazine, phenazopyridine, phenobarbitol, phenoxybenzamine, phentermine, phentolamine, phenyloin, physostigmine, pilocarpine, pimozide, pindolol, pipemidic acid, pirbuterol, piroxicam, polymixin, posaconazole, practolol, pramoxine, pravastatin, prazosin, prednisolone, prenalterol, primaquine, primidolol, prizidilol, procainamide, procaine, procaterol, prochlorperazine, promazine, promethazine, propanolol, proparacaine, proparacaine, propoxyphene, propranolol, pyrazinamide, pyrimethamine, pyroxidine, quazepam, quetiapine fumarate, quinapril, quinidine, quinine, quinterenol, raloxifen hydrochloride, ramipril, ranitidine, ranitidine hydrochloride, ravuconazole, retinoic acid, ribavarin, riboflavin, rifabutin, rifampin, rimiterol, risperidone, ritodrine, rocuronium, rosiglitazone, salmeterol, saperconazole, scopolamine, sertaconazole, sertraline, sibutramine, simvastatin, soterenol, sotolol, stavudine, sufentanil, sulconazole, sulfadiazine, sulfamethizole, sulfamethoxazole, sulfasalazine, sulfasoxazole, sulfinolol, sulfonterol, suloctidil, sumatriptan succinate, tacrine, tamoxifen, tamulosin hydrochloride, tazolol, temazepam, tenormin, tentrahydrozoline, terazosin, terbinafine, terbutaline, terconazole, terfenadine, tetracaine, tetracycline, tetrahydrazoline, tetrahydrolipstatin, theophylline, thiabendazole, thiamine, thiethylperazine, thioguanine, thioridazine, thiothixene, tiarnenidine, ticlopidine, timolol, tinazoline, tioconazole, tiotidine, tiprenolol, tipropidil, tocainide, tolamolol, tolazamide, tolazoline, tolmetin, tolteridine, toprimate, tramadol, tramazoline, trazodone, triamcinolone acetonide, triamterene, triazolam, trifluoroperazine, triflupromazine, trihexyphenidyl, trimeprazine, trimethoprin, trimetrexate, trimipramine, triplenamine, troglitazone, troleandomycin, tropicamide, tubocurarine, uracil mustard, valacyclovir hydrochloride, valconazole, valproic acid, valsartan, vecuronium, venlafaxine, verapamil, vidarabine, vinblastine, vincristine, vinorelbine, voriconazole, warfarin, xylometazoline, zinoconazole, zoficonazole and zolmitriptan.

The preferred pharmaceutical compounds which may be advantageously employed to produce the enhanced propertied human health pharmaceutical compounds of this invention include, but are not limited to, acrivistine, adapalene, aletamine, aliconazole, amiodarone, amitriptyline, amoxapine, amoxicillin, amphetamine, amrinone, arthrocine, astemizole, atorvastatin, atropine, becliconazole, benazepril, benzatropine, benzphetamine, beperiden, betahistine, bicalutamide, bisacodyl, brolaconazole, bromopheniramine, bupivacaine, buprenorphine, bupropion, caffeine, cafiminol, carbamazepam, cefachlor, cefadroxil, centerdrine, chlordiazepoxide, chloroprocaine, chloroquine, ciprofloxacin, citalopram, clemastine, clobesol, clomiphene, clonazepam, clonidine, clotrimazole, cloxacillin, clozapine, colchicine, croconazole, cyclobenzaprine, cyclopentolate, cyproheptadine, demethylimipramine, democonazole, deprenil, desipramine, dicodid, dicyclomine, diethylproprion, diltiazem, diphenidol, diphenoxylate hydrochloride, diphenhydramine doconazole, donezapil hydrochloride, doxapram, doxazosin, doxepin, eberconazole, econazole, enalapril, enoxacin, etintidine, famciclovir, fentanyl, fenfluramine, fenticonazole, flavoxate, fluazepam, fluconazole, fludorex, fluoxetine hydrochloride, flurbiprofen, fluticasone propionate, gabapentin, gemfibrozil, genaconazole, halazepam, haloperidol, hydroxyitraconazole, hydroxyzine, ibuprofen, indomethacin, iodoquinol, isoconazole, ketoprofen, ketorolac, lansoprazole, levomethadyl, lomefloxacin, loperidine hydrochloride, loratadine, lorazepam, lovastatin, mazindol, mechlorethamine, meperidine, mepivacaine, methadone, methimazole, methylphenidate, metronidazole, miconazole, minoxidil, molindone, monopril, monozid, naftifine, naphazoline, naproxen, nefazodone, neticonazole, nifedinpine, nifurantin, norfloxacin, olanzapine, omeprazole, omoconazole, orconazole, orphenadrine, oxaprozin, oxiconazole, oxmetidine, oxybutynin, oxycodone, oxymetazoline, papaverine, parconazole, paroxetine, pentazocine, perphenazine, phenoxybenzamine, phentermine, pilocarpine, pimozide, piroxicam, posaconazole, pramoxine, prazosin, procaine, proparacaine, propoxyphene, pyrazinamide, pyroxidine, quinapril, quinidine, ravuconazole, retinoic acid, risperidone, scopolamine, sertaconazole, sertraline, sibutramine, simvastatin, sufentanil, sulconazole, sulfamethizole, tacrine, tamoxifen, temazepam, terazosin, terbinafine, tetrahydrazoline, thiabendazole, ticlopidine, timolol, tioconazole, tocainide, tolazoline, tolteridine, tramadol, triamterene, trihexyphenidyl, troglitazone, troleandomycin, tropicamide, valconazole, valproic acid, verapamil, voriconazole, warfarin, xylometazoline and zinoconazole.

The pharmaceutical compounds which may be advantageously employed to produce the enhanced propertied veterinary pharmaceutical compounds of this invention include, but are not limited to, acepromazine, acetaminophen, acetazolamide, acetohydroxamic acid, acetylcysteine, acetylcysteine, acyclovir, aggrastat, albendazole, albuterol, allopurinol, altrenogest, aminocaproic acid, aminopentamide, aminophylline, aminopropazine, amiodarone, amitraz, amitriptyline, amlodipine, amoxicillin, ampicillin, amprolium, amrinone, apomorphine, apramycin, ascorbic acid, aspirin, atenolol, atipamezole, atropine, aurothioglucose, azaperone, azathioprine, benazepril, betamethasone, boldenone, bromocriptine, buprenorphine, buspirone, butorphanol, captopril, carbenicillin, carnitine, carprofen, cefadroxil, cefazolin, cefoperawne, cefotaxime, cefoxitin, ceftiofur, ceftriaxone, cephalexin, cephalothin, cephapirin, chloramphenicol, chlorothiazide, chlorpheniramine, chlorpromazine, chlorpropamide, cilastatin, cimetidine, ciprofloxacin, cisapride, clavulanate, clemastine, clenbuterol, clioquinol, clomipramine, clonazepam, cloprostenol, clorazepate, clorsulon, cloxacillin, codeine, colchicine, cyclophosphamide, cyproheptadine, cytarabine, cythioate, danazol, decoquinate, desoxycorticosterone Piv, detomidine, dexamethasone, dexpanthenol, diazoxide, dichlorphenamide, dicloxacillin, diethylcarbamazine, difloxacin, digitoxin, digoxin, dihydrotachysterol, diltiazem, dimenhydrinate, dimercaprol, dinoprost, diphenhydramine, diphenoxylate, disopyramide, dobutamine, dopamine, doramectin, doxapram, doxepin, doxorubicin, doxylamine, droperidol, edetate calcium, enalapril, enrofloxacin, ephedrine, epinephrine, esmolol, estradiol, ethacrynic acid, ethylisobutrazine, etidronate, etodolac, famotidine, febantel, fenbendazole, fenprostalene, fentanyl, fipronil, florfenicol, fluconazole, flucytosine, fludrocortisone, flumazenil, flumethasone, flunixin, fluprostenol, fomepizole-4-MP, furosemide, glipizide, glycopyrrolate, guaifenesin, hetacillin, hydralazine, hydrochlorothiazide, hydrocodone, hydrocortisone, hydroxyurea, hydroxyzine, imidacloprid, imidocarb, imipenem, imipramine, isopropamide, isoproterenol, isotretinoin, isoxsuprine, itraconazole, ketamine, ketoconazole, ketoprofen, levamisole, levothyroxine, lidocaine, lincomycin, liothyronine, lomustine, lufenuron, mechlorethamine, meclizine, meclofenamic acid, medetomidine, medroxyprogesterone, megestrol, melphalan, meperidine, mephenyloin, mercaptopurine, methenamine, methimazole, methionine, methocarbamol, methotrexate, methylprednisolone, metoclopramide, metoprolol, metronidazole, mexiletine, mibolerone, midazolam, misoprostol, morantel, morphine, naloxone, naltrexone, nandrolone, naproxen, nitrofurantoin, omeprazole, orbifloxacin, ormetoprim, oxacillin, oxazepam, oxfendazole, oxibendazole, oxybutynin, oxymorphone, penicillamine, penicillin G, penicillin V, pentazocine, pentoxifylline, phenoxybenzamine, phenylephrine, phenylpropanolamine, phenyloin, phytonadione, piperazine, pirlimycin, piroxicam, pralidoxime, prazosin, prednisolone, prednisone, primidone, procainamide, promazine, propofol, propranolol, prostaglandin E1, pyrantel, pyrilamine, pyrimethamine, quinacrine, quinidine, ranitidine, selegiline, stanozolol, sulfachlorpyridazine, sulfadiazine, sulfadimethoxine, sulfamethoxazole, sulfasalazine, terbutaline, testosterone, theophylline, thiabendazole, thiamine, thiotepa, tiamulin, ticarcillin, tiletamine, tiopronin, tocainide, tolazoline, triamcinolone, trimeprazine, trimethoprim, tripelennamine, ursodiol, valproic acid, verapamil, vinblastine, vincristine, warfarin, xylazine and yohimbine.

The preferred pharmaceutical compounds which may be advantageously employed to produce the enhanced propertied veterinary pharmaceutical compounds of this invention include, but are not limited to, acepromazine, albendazole, aminocaproic acid, aminophylline, aminopropazine, amiodarone, amitriptyline, amprolium, atipamezole, atropine, azaperone, benazepril, buspirone, captopril, cephapirin, chlorpheniramine, cisapride, clemastine, clioquinol, clomipramine, cyproheptadine, detomidine, diethylcarbamazine, diltiazem, diphenhydramine, diphenoxylate, disopyramide, doxapram, doxepin, doxylamine, droperidol, enalapril, febantel, fenbendazole, fentanyl, fluconazole, guaifenesin, hetacillin, hydrocodone, hydroxyzine, imidacloprid, itraconazole, ketamine, ketoprofen, levamisole, lidocaine, lincomycin, lomustine, mechlorethamine, meclizine, meclofenamic acid, meperidine, mercaptopurine, methenamine, methotrexate, mexiletine, mibolerone, morantel, naltrexone, omeprazole, ormetoprim, oxazepam, oxybutynin, pentazocine, piroxicam, primidone, procainamide, promazine, prostaglandin E1, pyrantel, quinacrine, quinidine, selegiline, sulfachlorpyridazine, sulfadiazine, sulfamethoxazole, theophylline, thiabendazole, tiamuLin, tiletamine, tocainide, valproic acid, verapamil, vincristine, warfarin and xylazine.

A first embodiment of this invention relates to a pharmaceutical moiety represented by Z¹(X¹)ₘ wherein

X¹ is an oxygen atom, a sulfur atom, a phosphorous atom or a nitrogen atom attached to Z¹,

Z¹ represents the remainder of said pharmaceutical moiety,

m is 1, and

Z¹(X¹)ₘ—H represents the complete pharmaceutical compound, said pharmaceutical moiety Z¹(X¹)ₘ being substituted with a second moiety on X¹, said second moiety comprising a substituent which enhances or changes the properties of said pharmaceutical compound, said substituent having the formula

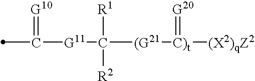

wherein

G¹⁰, G¹¹ and G²⁰ are each independently an oxygen atom or a sulfur atom,

G²¹ is an oxygen atom, a sulfur atom or NR³,

q is 0,

t is 0 or 1,

●- represents the connection point of said substituent to said pharmaceutical moiety Z¹(X¹)ₘ,

Z²(X²)_{q} is a hydrogen atom, alkyl, alkylcarbonyloxyalkyl, alkylcarbonyl, alkenylcarbonyl, alkynylcarbonyl, hydroxyalkyl, alkylsulfonylalkyl, alkylcarbonylaminoalkyl, arylcarbonylaminoalkyl, heteroarylcarbonylaminoalkyl, haloalkyl, alkenyl, acetylaminoalkenyl, alkylcarbonylaminoalkenyl, arylcarbonylaminoalkenyl, heteroarylcarbonylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioaLkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and alkylNR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl, or heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N-G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)+M⁻}, OR³, S(O)ⱼR³ or SO₂NR³R⁴ when both q and t are 0 wherein M⁻ is halo, hydroxy, alkoxy or the anion of a carboxylic acid and j is 0, or 2,

R¹ is

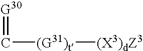

wherein

G³⁰ is an oxygen atom or a sulfur atom,

G³¹ is an oxygen atom, a sulfur atom or NR³,

d is 0,

t′ is 0 or 1,

Z³(X³)_{d}, when d is 0 and t′ is 1, is a hydrogen atom, alkyl, alkylcarbonyloxyalkyl, alkylcarbonyl, alkylcarbonylalkyl, alkenylcarbonyl, alkenylcarbonylalkyl, alkynylcarbonyl, alkynylcarbonylalkyl, hydroxyalkyl, alkylsulfonylalkyl, alkylcarbonylaminoalkyl, arylcarbonylaminoalkyl, heteroarylcarbonylalkylamino, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, arylcarbonyloxyalkyl, arylcarbonylalkyl, aroxycarbonylalkyl, or aryl, arylcarbonyloxyalkyl, arylcarbonylalkyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkylcarbonyloxyalkyl, aralkylcarbonylalkyl, aralkoxycarbonylalkyl, aralkenyl, aralkynyl, arcycloalkyl, aroxyalkyl, or aralkyl, aralkylcarbonyloxyalkyl, aralkylcarbonylalkyl, aralkoxycarbonylalkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyloxyalkyl, heteroarylcarbonylalkyl, heteroaroxycarbonylalkyl, or heteroaryl, heteroarylcarbonyloxyalkyl, heteroarylarylcarbonylalkyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl, or heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴ wherein j is 0, 1 or 2, Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylaLienyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl, aroxycarbonylalkyl, or arylcarbonyl, aralkylcarbonyl, aralkenylcarbonyl, aralkynylcarbonyl, aroxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl, or heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo; alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring, or

the pharmaceutically acceptable salts, isomers, tautomers, enantiomers and mixtures thereof.

A second embodiment of this invention relates to a pharmaceutical moiety represented by Z¹(X¹)ₘ wherein

X¹ is an oxygen atom, a sulfur atom, a phosphorous atom or a nitrogen atom attached to Z¹,

Z¹ represents the remainder of said pharmaceutical moiety,

m is 1, and

Z¹(X¹)ₘ—H represents the complete pharmaceutical compound, said pharmaceutical moiety Z¹(X¹)ₘ being substituted with a second moiety on X¹, said second moiety comprising a substituent which enhances or changes the properties of said pharmaceutical compound, said substituent having the formula

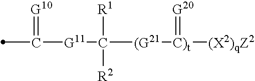

wherein

G¹⁰, G¹¹ and G²⁰ are each independently an oxygen atom or a sulfur atom,

G²¹ is an oxygen atom, a sulfur atom or NR³,

q is 0 or 1,

●- represents the connection point of said substituent to said pharmaceutical moiety Z¹(X¹)ₘ,

X² is an oxygen atom, a sulfur atom, a phosphorous atom, a nitrogen atom or a carbon atom attached to Z²,

t is 0 or 1,

Z²(X²)_{q}(C(=G²⁰)G²¹)ₜ is a second pharmaceutical moiety when q is 1 wherein Z²(X²)_{q}(C(=G²⁰)G²¹)ₜ or Z²(X²)_{q}(C(=G²⁰)G²′)ₜ—H represents the second pharmaceutical,

Z² represents the remainder of said second pharmaceutical moiety,

R¹ is

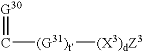

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)_{t′} is a second alternative pharmaceutical moiety when d is 1 wherein Z³(X³)_{d}(G³′)ₜ—H represents the second alternative pharmaceutical,

Z³ represents the remainder of said second alternative pharmaceutical moiety,

Z³(X³)_{d}, when d is 0 and t′ is 1, is a hydrogen atom, alkyl, alkylcarbonyloxyalkyl, alkylcarbonyl, alkylcarbonylalkyl, alkenylcarbonyl, alkenylcarbonylalkyl, alkynylcarbonyl, alkynylcarbonylalkyl, hydroxyalkyl, alkylsulfonylalkyl, alkylcarbonylaminoalkyl, arylcarbonylaminoalkyl, heteroarylcarbonylalkylamino, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkeiiyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, arylcarbonyloxyalkyl, arylcarbonylalkyl, aroxycarbonylalkyl, or aryl, arylcarbonyloxyalkyl, arylcarbonylalkyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkylcarbonyloxyalkyl, aralkylcarbonylalkyl, aralkoxycarbonylalkyl, aralkenyl, aralkynyl, arcycloalkyl, aroxyalkyl, or aralkyl, aralkylcarbonyloxyalkyl, aralkylcarbonylalkyl, aralkoxycarbonylalkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyloxyalkyl, heteroarylcarbonylalkyl, heteroaroxycarbonylalkyl, or heteroaryl, heteroarylcarbonyloxyalkyl, heteroarylarylcarbonylalkyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterQcyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl, or heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴ wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl, aroxycarbonylalkyl, or arylcarbonyl, aralkylcarbonyl, aralkenylcarbonyl, aralkynylcarbonyl, aroxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloallcynyl, alkoxy, haloalkoxy, SO₂NR³R⁴ and NR³R⁴, heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl, or heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring,

(q+d) is 1, or

the pharmaceutically acceptable salts, isomers, tautomers, enantiomers and mixtures thereof.

A third embodiment of this invention relates to a pharmaceutical compound of formula (I)

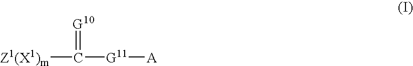

wherein

A is

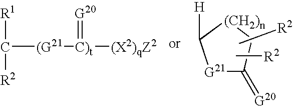

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m, q and t are each independently 0 or 1,

n is 1 or 2,

Z¹(X¹)ₘ is a pharmaceutical moiety when m is 1 wherein Z¹(X¹)ₘ—H represents the pharmaceutical,

Z²(X²)_{q}(C(=G²⁰)G²¹)ₜ is a pharmaceutical moiety when q is 1 wherein Z²(X²)_{q}(C(=G²⁰)G²¹)ₜ—H or Z²(X²)_{q}(C(=G²⁰)G²′)ₜ represents the pharmaceutical,

Z¹(X¹)ₘ, when m is 0, is a hydrogen atom, halo, alkyl, alkylcarbonyloxyalkyl, alkylcarbonyl, hydroxyalkyl, alkylsulfonylalkyl, acetylaminoalkyl, alkylcarbonylaminoalkyl, arylcarbonylaminoalkyl, heteroarylcarbonylaminoalkyl, haloalkyl, alkenyl, acetylaminoalkenyl, alkylcarbonylaminoalkenyl, arylcarbonylaminoalkenyl, heteroarylcarbonylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkenyl, alkoxyalkynyl, alkoxycarbonyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, OR³, S(O)ⱼR³, carboxyalkyl, carboxyalkenyl, carboxyalkynyl, aryl, arylcarbonyl, arylcarbonylalkyl, aroxycarbonyl, aroxycarbonylalkyl, or aryl, arylcarbonyl, arylcarbonylalkyl, aroxycarbonyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, aralkylcarbonyl, aralkenylcarbonyl, aralkylcarbonylalkyl, aralkenylcarbonylalkyl, or aralkyl, aralkenyl, aralkynyl, arcycloalkyl, aroxyalkyl, aralkylcarbonyl, aralkenylcarbonyl, aralkylcarbonylalkyl, aralkenylcarbonylalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyl, heteroarylcarbonylalkyl, heteroaroxycarbonyl, heteroaroxycarbonylalkyl, or heteroaryl, heteroarylcarbonyl, heteroaroxycarbonylalkyl, heteroaroxycarbonyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, heteroaralkylcarbonyl, heteroaralkylcarbonylalkyl, heteroaralkenylcarbonyl, heteroaralkenylcarbonylalkyl, or heteroaralkyl, heteroaralkenyl, heteroaralkynyl, heteroaralkylcarbonyl, heteroaralkylcarbonylalkyl, heteroaralkenylcarbonyl, heteroaralkenylcarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylalkyl, heterocyclylalkenyl, heterocyclylalkynyl, heterocyclylcarbonyl, heterocyclylcarbonylalkyl, heterocyclyloxycarbonyl, heterocyclyloxycarbonylalkyl, or heterocyclyl, heterocyclylalkyl, heterocyclylalkenyl, heterocyclylalkynyl, heterocyclylcarbonyl, heterocyclylcarbonylalkyl, heterocyclyloxycarbonyl, heterocyclyloxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴ wherein j is 0, 1 or 2,

Z²(X²)_{q} is a hydrogen atom, alkyl, alkylcarbonyloxyalkyl, alkylcarbonyl, alkenylcarbonyl, alkynylcarbonyl, hydroxyalkyl, alkylsulfonylalkyl, alkylcarbonylaminoalkyl, arylcarbonylaminoalkyl, heteroarylcarbonylaminoalkyl, haloalkyl, alkenyl, acetylaminoalkenyl, alkylcarbonylaminoalkenyl, arylcarbonylaminoalkenyl, heteroarylcarbonylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and alkylNR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl, or heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N-G²²)R² when q is 0 and t is 1,

G²² is OR³, OCOR³, S(O)ⱼR³, OS(O)ⱼR³⁷ NR³R⁴, OSO₂NR³R⁴, OP(—O)OR³NR³R⁴, OP(═O)(OR³)₂ or N═CR³R⁴,

j is 0, 1 or 2,

Z²(X²)_{q} is halo, NR³R⁴, {(NR³R⁴R⁵)⁺M⁻}, OR³, S(O)ⱼR³ or SO₂NR³R⁴ when both q and t are 0 wherein M⁻ is halo, hydroxy, alkoxy or the anion of a carboxylic acid and j is 0, 1 or 2,

R¹ is

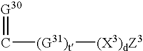

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)ₜ, is a pharmaceutical moiety when d is 1 wherein Z³(X³)_{d}(G³¹)_{t′}—H represents the pharmaceutical,

Z³(X³)_{d}, when d is 0 and t′ is 1, is a hydrogen atom, alkyl, alkylcarbonyloxyalkyl, alkylcarbonyl, alkylcarbonylalkyl, alkenylcarbonyl, alkenylcarbonylalkyl, alkynylcarbonyl, alkynylcarbonylalkyl, hydroxyalkyl, alkylsulfonylalkyl, alkylcarbonylaminoalkyl, arylcarbonylaminoalkyl, heteroarylcarbonylalkylamino, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, arylcarbonyloxyalkyl, arylcarbonylalkyl, aroxycarbonylalkyl, or aryl, arylcarbonyloxyalkyl, arylcarbonylalkyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkylcarbonyloxyalkyl, aralkylcarbonylalkyl, aralkoxycarbonylalkyl, aralkenyl, aralkynyl, arcycloalkyl, aroxyalkyl, or aralkyl, aralkylcarbonyloxyalkyl, aralkylcarbonylalkyl, aralkoxycarbonylalkyl, aralk-enyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyloxyalkyl, heteroarylcarbonylalkyl, heteroarbxycarbonylalkyl, or heteroaryl, heteroarylcarbonyloxyalkyl, heteroarylarylcarbonylalkyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaraLkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl, or heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴ wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

each R² is independently a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalky nyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl, aroxycarbonylalkyl, or arylcarbonyl, aralkylcarbonyl, aralkenylcarbonyl, aralkynylcarbonyl, aroxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl, or heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,

R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring, or

A is

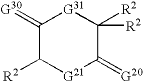

wherein each R², G²⁰, G²¹, G³⁰ and G³¹ are as previously defined,

provided that when both m and q are 0, A is

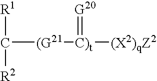

and

within the definition of R¹

d is 1,

G³⁰, G³¹, Z³, X³ and t′ are as previously defined and

Z³(X³)_{d}(G³¹)_{t′} is a pharmaceutical moiety wherein Z³(X³)_{d}(G³′)_{t′}—H represents the pharmaceutical, or

the pharmaceutically acceptable salts, isomers, tautomers, enantiomers and mixtures thereof.

In a preferred third embodiment of this invention, the pharmaceutical compound is represented by the compound of formula (I) wherein t is 1, m is 1, q is 0, (X¹) is a nitrogen atom and Z¹(X¹)ₘ—H is a pharmaceutical, or the pharmaceutically acceptable salts, isomers, tautomers, enantiomers and mixtures thereof.

In another preferred third embodiment of this invention, the pharmaceutical compound is represented by the compound of formula (I) wherein t is 1, m is 1, q is 0, (X¹)ₘ is a phosphorous, oxygen or sulfur atom and Z¹(X¹)ₘ—H is a pharmaceutical, or t is 1, m is 0, q is 1, (X²)_{q} is a phosphorous, oxygen or sulfur atom and Z²(X²)_{q}-[(C=G²⁰)-G²¹]ₜ—H is a pharmaceutical, or the pharmaceutically acceptable salts, isomers, tautomers, enantiomers and mixtures thereof.

In another preferred third embodiment of this invention, the pharmaceutical compound is represented by the compound of formula (I) wherein t is 1, m is 0, q is 1, (X²)_{q} is a carbon atom and Z²(X²)_{q}-[(C=G²⁰)-G²¹]ₜ-H is a pharmaceutical, or the pharmaceutically acceptable salts, isomers, tautomers, enantiomers and mixtures thereof.

In another preferred third embodiment of this invention, the pharmaceutical compound is represented by the compound of formula (I) wherein t is 0, m is 1, q is 0, (X¹)ₘ is a nitrogen, phosphorous, oxygen or sulfur atom and Z¹(X¹)ₘ—H is a pharmaceutical, or t is 0, m is 0, q is 1, (X²)_{q} is a nitrogen, phosphorous, oxygen, sulfur or carbon atom and Z²(X²)_{q}-[(C=G²⁰)-G²¹]ₜ-H is a pharmaceutical or Z²(X²)_{q}-[(C=G²⁰)-G²¹]ₜ is a pharmaceutical, or the pharmaceutically acceptable salts, isomers, tautomers, enantiomers and mixtures thereof.

In another preferred third embodiment of this invention, the pharmaceutical compound is represented by the compound of formula (I) wherein t is 0 or 1, m is 1, q is 1, (X¹)ₘ is a nitrogen, phosphorous, oxygen or sulfur atom, (X²)_{q} is a nitrogen, phosphorous, oxygen, sulfur or carbon atom, Z¹(X¹)ₘ—H is a pharmaceutical and Z²(X²)_{q}—(C=G²⁰-G²¹)ₜ-H or Z²(X²)_{q}-(C=G²⁰-G²¹)ₜ is a pharmaceutical, or the pharmaceutically acceptable salts, isomers, tautomers, enantiomers and mixtures thereof.

In another preferred third embodiment of this invention, the pharmaceutical compound is represented by the compound of formula (I) wherein A is

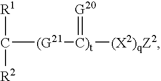

m and q are 0,

Z¹(X¹)ₘ and Z²(X²)_{q} are non-pharmaceutical moieties,

R¹ is

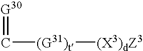

wherein

G³⁰ is an oxygen atom or a sulfur atom,

G³¹ is an oxygen atom, a sulfur atom or NR³,

d is 1,

t′ is 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G3³¹ is NR³, or a carbon atom attached to Z³ when t′ is 1 and G³¹ is an oxygen atom or a sulfur atom,

Z³(X³)_{d}(G³¹)_{t′} is a pharmaceutical moiety wherein Z³(X³)_{d}(G³¹)_{t′}-H represents the pharmaceutical, or

the pharmaceutically acceptable salts, isomers, tautomers, enantiomers and mixtures thereof.

In another preferred third embodiment of this invention, a pharmaceutical compound is represented by formula (I)

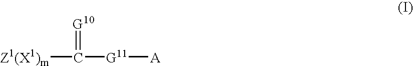

wherein

A is

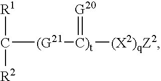

R¹ is

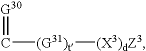

(d+m+q) is 1 or 2, or

the pharmaceutically acceptable salts, isomers, tautomers, enantiomers and mixtures thereof.

In another preferred third embodiment of this invention, a pharmaceutical compound is represented by formula (I)

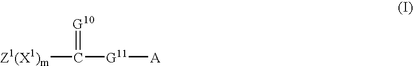

wherein

A is

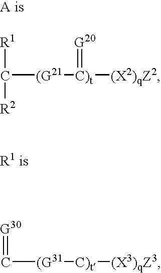

both q and t are 1,

X² is a carbon atom, or

the pharmaceutically acceptable salts, isomers, tautomers, enantiomers and mixtures thereof.

In another preferred third embodiment of this invention, a pharmaceutical compound is represented by formula (I)

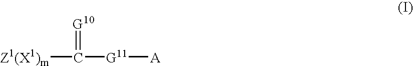

wherein

A is

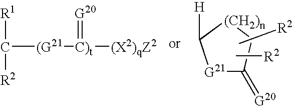

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m, q and t are each independently 0 or 1,

n is 1 or 2,

Z¹(X¹)ₘ is a pharmaceutical moiety when m is 1 wherein Z¹(X¹)ₘ—H represents the pharmaceutical,

Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²¹)ₜ-H represents the pharmaceutical,

Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C3-C₈)alkenyl, carboxycyclo(C₃-C₈)alkyl, carboxycyclo(C₃-C₈)alkenyl, cyclo(C3-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀))alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C3-Cs)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,

Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₋₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₉)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(Ca-CB)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)+M-}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,

R¹ is

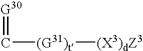

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)_{t′} is a pharmaceutical moiety when d is 1 wherein Z³(X³)_{d}(G³¹)_{t′}-H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclO(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₁-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₁₀)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀ alkyl, or a carbon ar C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkWxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₁₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

A is

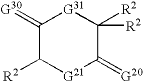

wherein each R², G²⁰, G²¹, G³⁰ and G³¹ are as previously defined,

provided that when both m and q are 0, A is

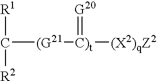

and

within the definition of R¹

d is 1,

G³⁰, G³¹, Z³, X³ and t′ are as previously defined and

Z³(X³)_{d}(G³¹)_{t′} is a pharmaceutical moiety wherein Z³(X³)_{d}(G³¹)_{t′}—H represents the pharmaceutical, or

the pharmaceutically acceptable salts, isomers, tautomers, enantiomers and mixtures thereof.

In a fourth embodiment of this invention, a human health pharmaceutical compound is represented by formula (I)

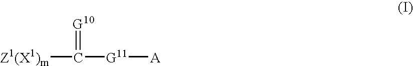

wherein

A is

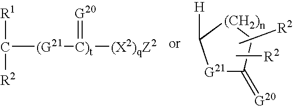

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m is 1,

q and t are each independently 0 or 1,

n is 1 or 2,

Z¹(X¹)ₘ is a pharmaceutical moiety when m is 1 wherein Z¹(X¹)ₘ—H represents the pharmaceutical selected from the group consisting of aletamine, amoxapine, amoxicillin, amphetamine, atorvastatin, benazepril, betahistine, bupropion, carbamazepam, cefachlor, cefadroxil, centerdrine, chlordiazepoxide, chloroquine, ciprofloxacin, clonazepam, clonidine, clozapine, demethylimipramine, deprenil, desipramine, enoxacin, etintidine, fenfluramine, fludorex, fluoxetine hydrochloride, gabapentin, lansoprazole, mepivacalne, metnylpneniclate, moincdone, napnazoline, norfloxacin, olanzapine, omeprazole, oxmetidine, paroxetine, phentermine, pimozide, piroxicam, posaconazole, prazosin, procaine, propanolol, proparacaine, quinapril, sertraline, sulfamethizole, tacrine, temazepam, terazosin, tetrahydrazoline, thiabendazole, timolol, tocainide, tolazoline, tramadol, triamterene, troglitazone and xylometazoline when X¹ is a nitrogen atom, or

the pharmaceutical selected from the group consisting of atorvastatin, atropine, bicalutamide, buprenorphine, cafiminol, clobesol, deprenil, doxazosin, enalapril, famciclovir, fluconazole, fluticasone propionate, genaconazole, haloperidol, hydroxyitraconazole, hydroxyzine, iodoquinol, loperidine hydrochloride, lorazepam, lovastatin, mazindol, metronidazole, oxycodone, quinidine, scopolamine, simvastatin, tramadol and voriconazole when X¹ is an oxygen atom, or

the pharmaceutical methimazole when X¹ is a sulfur atom,

Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²¹)ₜ—H represents the pharmaceutical,

Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₂-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl halo(C₁-C₁₀)alkoxy(C₂-C₁₀)alkynyl, (C₁-C₁₀)alkylthio(C₁-C₁₀) alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀) alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)⁺M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,

R¹ is

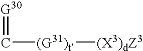

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)_{t′} is a pharmaceutical moiety when d is 1 wherein Z³(X³)_{d}(G³¹)_{t′}—H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C3-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C3-CS8)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀) alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR¹R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyll cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halO(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)-alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

A is

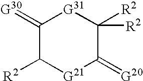

wherein each R², G²⁰, G²¹, G³⁰ and G³¹ are as previously defined,

provided that when both m and q are 0, A is

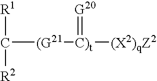

and

within the definition of R¹

d is 1,

G³⁰, G³¹, Z³, X³ and t′ are as previously defined and

Z³(X³)_{d}(G³¹)_{t′} is a pharmaceutical moiety wherein Z³(X³)_{d}(G³¹)_{t′}—H represents the pharmaceutical, or

the pharmaceutically acceptable salts, isomers, tautomers, enantiomers and mixtures thereof.

In a fifth embodiment of this invention, a human health pharmaceutical compound is represented by formula (I)

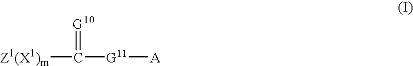

wherein

A is

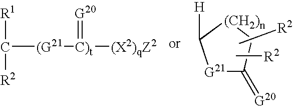

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m and t are each independently 0 or 1,

q is 1,

n is 1 or 2,

Z¹(X¹)ₘ is a pharmaceutical moiety when m is 1 wherein Z¹(X¹)ₘ—H represents the pharmaceutical,

Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²′)ₜ—H represents the pharmaceutical selected from the group consisting of acrivistine, aliconazole, amiodarone, amitriptyline, amoxapine, amrinone, astemizole, atropine, becliconazole, benzatropine, benzphetamine, beperiden, bisacodyl, brolaconazole, bromopheniramine, bupivacaine, caffeine, chloroprocaine, citalopram, clemastine, clomiphene, clotrimazole, colchicine, croconazole, cyclobenzaprine, cyclopentolate, cyproheptadine, democonazole, dicodid, dicyclomine, diethylproprion, diphenhydramine, diphenidol, diltiazem, diphenoxylate hydrochloride, doconazole, donezapil hydrochloride, doxapram, doxepin, eberconazole, econazole, fentanyl, fenticonazole, flavoxate, fluazepam, fluconazole, halazepam, hydroxyitraconazole, isoconazole, lansoprazole, levomethadyl, loratadine, mechlorethamine, meperidine, mepivacaine, methadone, methimazole, miconazole, minoxidil, naftifine, nefazodone, neticonazole, nifurantin, omoconazole, orconazole, orphenadrine, oxiconazole, oxybutynin, oxymetazoline, papaverine, parconazole, phenoxybenzamine, pilocarpine, pramoxine, propoxyphene, pyrazinamide, pyroxidine, ravuconazole, retinoic acid, risperidone, sertaconazole, sibutramine sufentanil, sulconazole, tamoxifen, terbinafine, ticlopidine, tioconazole, tolteridine, trihexyphenidyl, troleandomycin, tropicamide, valconazole, verapamil and zinoconazole when X² is a nitrogen atom, or

the pharmaceutical selected from the group consisting of fluconazole, genaconazole, hydroxyitraconazole, iodoquinol, lovastatin, mazindol, metronidazole, posaconazole, voriconazole and warfarin when X² is an oxygen atom, or

the pharmaceutical selected from the group consisting of adapalene, arthrocine, cloxacillin, flurbiprofen, gemfibrozil, hydroxyitraconazole, ibuprofen, indomethacin, ketoprofen, ketorolac, loratadine, monopril, oxaprozin, valproic acid and verapamil when X² is an carbon atom,

Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C3-Cs)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-Cs)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C3-Cs)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,

R¹ is

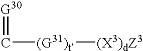

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)_{t′} is a pharmaceutical moiety when d is 1 wherein Z³(X³)_{d}(G³¹)_{t′}—H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C3-C₈)alkenyl(C₁-C₁₀)alkyl, carboxycyclo(C₃-C₁₀)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C3-Cs)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(c₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

A is

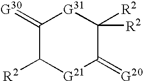

wherein each R², G²⁰, G²¹, G³⁰ and G³¹ are as previously defined,

provided that when both m and q are 0, A is

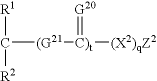

and

within the definition of R¹

dis 1,

G³⁰, G³¹, Z³, X³ and t′ are as previously defined and

Z³(X³)_{d}(G³¹)_{t′} is a pharmaceutical moiety wherein Z³(X³)_{d}(G3′)_{t′}—H represents the pharmaceutical, or

the pharmaceutically acceptable salts, isomers, tautomers, enantiomers and mixtures thereof.

In a sixth embodiment of this invention, a veterinary pharmaceutical compound is represented by formula (I)

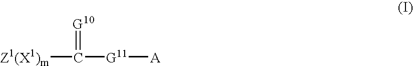

wherein

A is

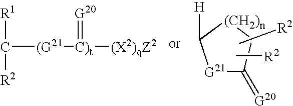

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m is 1,

q and t are each independently 0 or 1,

n is 1 or 2,

Z¹(X¹)ₘ is a pharmaceutical moiety when m is 1 wherein Z¹(X¹)ₘ-H represents the pharmaceutical selected from the group consisting of albendazole, aminocaproic acid, aminophylline, amprolium, atipamezole, benazepril, cisapride, detomidine, disopyramide, enalapril, febantel, fluconazole, imidacloprid, ketamine, lidocaine, lincomycin, lomustine, mechlorethamine, meclofenamic acid, mercaptopurine, methotrexate, mexiletine, ormetoprim, piroxicam, primidone, procainamide, prostaglandin E1, quinacrine, quinidine, sulfachlorpyridazine, sulfadiazine, sulfamethoxazole, theophylline, thiabendazole, tiletamine, tocainide, vincrist-ine and xylazine when X¹ is a nitrogen atom, or

the pharmaceutical selected from the group consisting of clioquinol, enalapril, guaifenesin, mibolerone, oxazepam, prostaglandin E1 and warfarin when X¹ is an oxygen atom,

Z²(X²)_{q}(C(=G²⁰)G²¹)ₜ is a pharmaceutical moiety when q is 1 wherein Z²(Y²)_{q}(C(=G²⁰)G²¹), or Z²(X²)_{q}(C(=G²O)G²′)ₜ—H represents the pharmaceutical,

Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₈-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(—S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)+M-}, OR³, S(O)ⱼR³ or SO₂NR³R⁴ when both q and t are 0 wherein M⁻ is halo, hydroxy, (CI-Cg)alkoxy or the anion of a carboxylic acid and j is 0, 1 or 2,

R¹ is

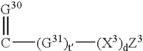

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)ₜ is a pharmaceutical moiety when d is 1 wherein Z³(X³)_{d}(G³¹)_{t′}—H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C3-Ca)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-Cs)alkenyl(C₂-C₁₀)alkynyl, carboxycyclo(C₃-CB)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-Cs)alkyl(C₂-C₁₀)alkvnyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)-alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents indepeindently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C3-Cg)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alk-enyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C4)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarykarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy; nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(Ca-C8)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C3-Cs)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkvnyl, (C₁-C₁₀)alkloxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

A is

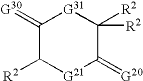

wherein each R², G²⁰, G²¹, G³⁰ and G³¹ are as previously defined,

provided that when both m and q are 0, A is

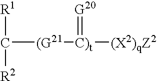

and

within the definition of R¹

d is 1,

G³⁰, G³¹, Z³, X³ and t′ are as previously defined and

Z³(X³)_{d}(G³¹)ₜ is a pharmaceutical moiety wherein Z³(X³)_{d}(G³′)ₜ—H represents the pharmaceutical, or

the pharmaceutically acceptable salts, isomers, tautomers, enantiomers and mixtures thereof.

In a seventh embodiment of this invention, a veterinary pharmaceutical compound is represented by formula (I)

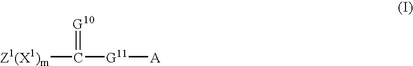

wherein

A is

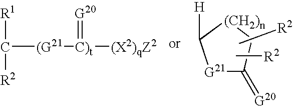

G¹⁰, G¹¹ and G²⁰ are each independently an oxygen atom or a sulfur atom,

G²¹ is an oxygen atom, a sulfur atom or NR³,

X¹ is an oxygen atom, a sulfur atom, a phosphorous atom or a nitrogen atom attached to Z¹,

X² is an oxygen atom, a sulfur atom, a phosphorous atom, a nitrogen atom or a carbon atom attached to Z²,

m and t are each independently 0 or 1,

q is 1,

n is 1 or 2,

Z¹(X¹)ₘ is a pharmaceutical moiety when m is 1 wherein Z¹(X¹)ₘ—H represents the pharmaceutical,

Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²¹)ₜ—H represents the pharmaceutical selected from the group consisting of acepromazine, albendazole, aminopropazine, amiodarone, amitriptyline, atropine, azaperone, buspirone, captopril, cephapirin, chlorpheniramine, clemastine, clomipramine, cyproheptadine, diethylcarbamazine, diltiazem, diphenhydramine, diphenoxylate, doxapram, doxepin, doxylamine, droperidol, febantel, fenbendazole, fentanyl, hetacillin, hydrocodone, hydroxyzine, itraconazole, levamisole, meclizine, meperidine, methenamine, morantel, naltrexone, omeprazole, oxybutynin, pentazocine, piroxicam, primidone, procainamide, promazine, pyrantel, selegiline, thiabendazole, tiamulin and verapamil when X² is a nitrogen atom, or

the pharmaceutical selected from the group consisting of clioquinol, guaifenesin, mibolerone, oxazezam and warfarin when X² is an oxygen atom, or

the pharmaceutical selected from the group consisting of ketoprofen and valproic acid when X² is a carbon atom,

Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C3-Ca)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀) alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alken-ylcarbonyl, ar(C₂-C₁₀)alkenycarbonyl(C₁-C₁₀)alkyl, or ar(C₁-C₁₀)alkyl, ar(C₂-C₁₀)alkenyl, ar(C₂-C₁₀) alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C3-Cs)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, hetercar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,

R¹ is

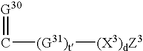

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³′ is an oxygen atom or a sulfur atom,

Z³(X³)_{d}(G³¹)_{t′} is a pharmaceutical moiety when d is 1 wherein Z³(X³)_{d}(G³¹)ₜ—H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C3-Cs)alkyl(C₁-C₁₀)alkyl, carboxycyclo(C₃-C₈)alkyl(C₂-C₁₀)alkenyl, carboxycyclo(C3-Cs)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, aryarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arCyclo(C3-Cs)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀))akyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀) alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein i is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C3-CB)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀) alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alke nyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl; (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀) alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈) alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alenyl, (C₂-C₁₀)alkyriyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

A is

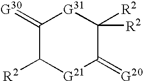

wherein each R², G²⁰, G²¹, G³⁰ and G³¹ are as previously defined,

provided that when both m and q are 0, A is

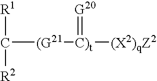

and

within the definition of R¹

d is 1,

G³⁰, G³¹, Z³, X³ and t′ are as previously defined and

Z³(X³)_{d}(G3′)_{t′} is a pharmaceutical moiety wherein Z³(X³)_{d}(G³′)_{t′}—H represents the pharmaceutical, or

the pharmaceutically acceptable salts, isomers, tautomers, enantiomers and mixtures thereof.

In an eighth embodiment of this invention, the pharmaceutical compound is represented by formula (I)

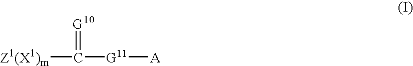

wherein

A is

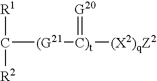

G¹⁰, G¹¹ and G²⁰ are each independently an oxygen atom or a sulfur atom,

G²¹ is an oxygen atom, a sulfur atom or NR³,

X¹ is a nitrogen atom attached to Z¹,

X² is an oxygen atom, a sulfur atom, a phosphorous atom, a nitrogen atom or a carbon atom attached to Z²,

m is 1,

q and t are each independently 0 or 1,

Z¹(X¹)ₘ is a pharmaceutical moiety selected from

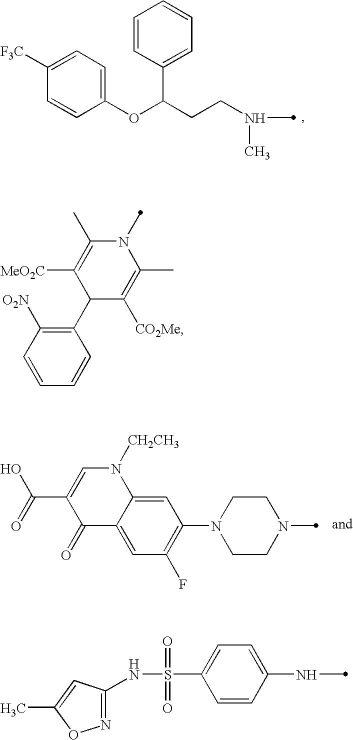

wherein Z¹(X¹)ₘ—H represents the respective pharmaceutical selected from

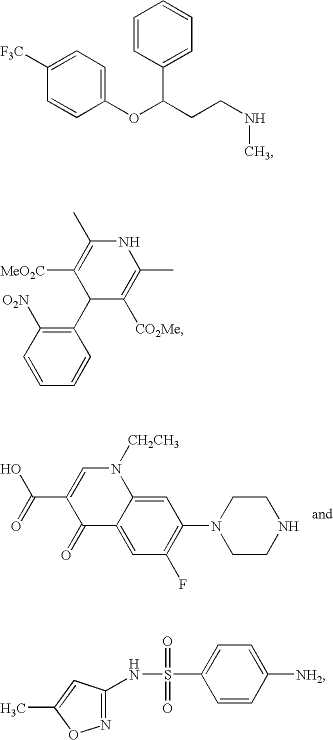

-● represents the connection point between said pharmaceutical moiety and the moiety represented by

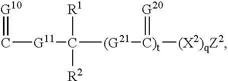

Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²¹)ₜH represents the pharmaceutical,

Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀) alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, R³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₁-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C2-C o)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,

Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(—O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydrqxy, cyano, (C₁-C₁₀)alkyl, cyclo(C₃-C₈)alkyl, (C2-Cto)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀) alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and N—R³R⁴, and C(═N-G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)⁺M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,

R¹ is

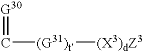

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)_{t′} is a pharmaceutical moiety when d is 1 wherein Z³(X³)_{d}(G³′)ₜ—H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀) alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀) alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)a kynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₁-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

(d+q)is 0 or 1,

R² is a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀) alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynylv (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀) alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C3-Cs)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

the pharmaceutically acceptable salts, isomers, tautomers, enantiomers and mixtures thereof.

In a ninth embodiment of this invention, the pharmaceutical-compound is represented by formula (I)

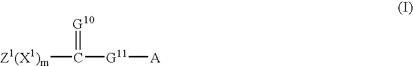

wherein

A is

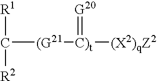

G¹⁰, G¹¹ and G²⁰ are each independently an oxygen atom or a sulfur atom,

G²¹ is an oxygen atom, a sulfur atom or NR³,

X¹ is an oxygen atom attached to Z¹,

X² is an oxygen atom, a sulfur atom, a phosphorous atom, a nitrogen atom or a carbon atom attached to Z²,

m is 1,

q and t are each independently 0 or 1,

Z¹(X¹)ₘ is a pharmaceutical moiety selected from

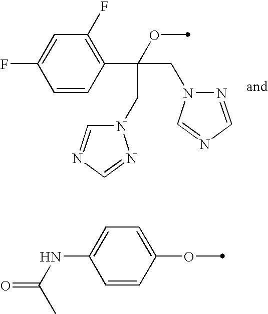

wherein Z¹(X¹)ₘ—H represents the respective pharmaceutical selected from

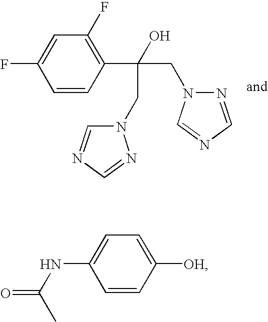

-● represents the connection point between said pharmaceutical moiety and the moiety represented by

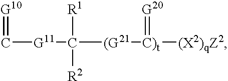

Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²¹)ₜ—H represents the pharmaceutical,

Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₉)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,

Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)Galkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloal(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C2-Cto)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, lieteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,

G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,

j is 0, 1 or 2,

Z²(X²)_{q} is halo, NR³R⁴, {(NR³R⁴R⁵)⁺M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,

R¹ is

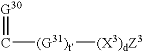

wherein

G³⁰ is an oxygen atom or a sulfur atom,

G³¹ is an oxygen atom, a sulfur atom or NR³,

t′ and d are each independently 0 or 1,

X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,

Z³(X³)_{d}(G³¹)ₜ is a pharmaceutical moiety when d is 1 wherein Z³(X³)_{d}(G³′)ₜ—H represents the pharmaceutical,

Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, Cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxyycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,

Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,

(d+q)is 0 or 1,

R² is a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C3-CB)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C3-Cg)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C4)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,

R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or

the pharmaceutically acceptable salts, isomers, tautomers, enantiomers and mixtures thereof.

Another aspect of this invention relates to pharmaceutical compositions comprising a pharmaceutical compound of this invention and a pharmaceutically acceptable carrier. Preferably, the composition contains from about 0.1% to about 99% by weight of said pharmaceutical compound depending on the host treated, the disease and the particular mode of administration.

Still another aspect of this invention relates to a method of controlling pain or disease symptoms in a warm-blooded animal exhibiting pain or disease symptoms comprising administering thereto a pharmaceutically effective amount of a compound or a composition comprising a compound of this invention and a pharmaceutically acceptable carrier.

In all embodiments of this invention, the term “alkyl” includes both branched and straight chain alkyl groups. Typical alkyl groups are methyl, ethyl, it-propyl, isopropyl, n-butyl, sec-butyl, isobutyl, tert-butyl, n-pentyl, isopentyl, n-hexyl, n-heptyl, isooctyl, nonyl, decyl, undecyl, dodecyl, tetradecyl, hexadecyl, octadecyl, eicosyl and the like.

The term “halo” refers to fluoro, chloro, bromo or iodo.

The term “haloalkyl” refers to an alkyl group substituted with one or more halo groups, for example chloromethyl, 2-bromoethyl, 3-iodopropyl, trifluoromethyl, perfluoropropyl, 8-chlorononyl and the like.

The term “cycloalkyl” refers to a cyclic aliphatic ring structure, optionally substituted with alkyl, hydroxy and halo, such as cyclopropyl, methylcyclopropyl, cyclobutyl, 2-hydroxycyclopentyl, cyclohexyl, 4-chlorocyclohexyl, cycloheptyl, cyclooctyl and the like.

The term “alkylcarbonyloxyalkyl” refers to an ester moiety, for example acetoxymethyl, n-butyryloxyethyl and the like.

The term “alkynylcarbonyl” refers to an alkynylketo functionality, for example propynoyl and the like.

The term “hydroxyalkyl” refers to an alkyl group substituted with one or more hydroxy groups, for example hydroxymethyl, 2,3-dihydroxybutyl and the like.

The term “alkylsulfonylalkyl” refers to an alkyl group substituted with an alkylsulfonyl moiety, for example mesylmethyl, isopropylsulfonylethyl and the like.

The term “alkylsulfonyl” refers to a sulfonyl moiety substituted with an alkyl group, for example mesyl, n-propylsulfonyl and the like.

The term “acetylaminoalkyl” refers to an alkyl group substituted with an amide moiety, for example acetylaminomethyl and the like.

The term “acetylaminoalkenyl” refers to an alkenyl group substituted with an amide moiety, for example 2-(acetylamino)vinyl and the like.

The term “alkenyl” refers to an ethylenicaliy unsaturated hydrocarbon group, straight or branched chain, having 1 or 2 ethlylenic bonds, for example vinyl, allyl, 1-butenyl, 2-butenyl, isopropenyl, 2-pentenyl and the like.

The term “haloalkenyl” refers to an alkenyl group substituted with one or more halo groups.

The term “cycloalkenyl” refers to a cyclic aliphatic ring structure, optionally substituted with alkyl, hydroxy and halo, having 1 or 2 ethylenic bonds such as methylcyclopropenyl, trifluoromethylcyclopropenyl, cyclopentenyl, cyclohexenyl, 1,4-cyclohexadienyl and the like.

The term “alkynyl” refers to an unsaturated hydrocarbon group, straight or branched, having 1 or 2 acetylenic bonds, for example ethynyl, propargyl and the like.

The term “haloalkynyl” refers to an alkynyl group substituted with one or Smore halo groups.

The term “alkylcarbonyl” refers to an alkylketo functionality, for example acetyl, n-butyryl and the like.

The term “alkenylcarbonyl” refers to an alkenylketo functionality, for example, propenoyl and the like.

The term “aryl” refers to phenyl or naphthyl which may be optionally substituted. Typical aryl substituents include, but are not limited to, phenyl, 4-chlorophenyl, 4-fluorophenyl, 4-bromophenyl, 3-nitrophenyl, 2-methoxyphenyl, 2-methylphenyl, 3-methyphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl and 2-iodo-4-methylphenyl.

The term “heteroaryl” refers to a substituted or unsubstituted 5 or 6 membered unsaturated ring containing one, two or three heteroatoms, preferably one or two heteroatoms independently selected from oxygen, nitrogen and sulfur or to a bicyclic unsaturated ring system containing up to 10 atoms including one heteroatom selected from oxygen, nitrogen and sulfur. Examples of heteroaryls include, but is not limited to, 2-, 3- or 4-pyridinyl, pyrazinyl, 2-, 4-, or 5-pyrimidinyl, pyridazinyl, triazolyl, imidazolyl, 2- or 3-thienyl, 2- or 3-furyl, pyrrolyl, oxazolyl, isoxazolyl, thiazolyl, isothiazolyl, oxadiazolyl, thiadiazolyl, quinolyl and isoquinolyl. The heterocyclic ring may be optionally substituted with up to two substituents.

The term “aralkyl” is used to describe a group wherein the alkyl chain can be branched or straight chain with the aryl portion, as defined hereinbefore, forming a terminal portion of the aralkyl moiety. Examples of aralkyl groups include, but are not limited to, optionally substituted benzyl, phenethyl, phenpropyl and phenbutyl such as 4-chlorobenzyl, 2,4-dibromobenzyl, 2-methylbenzyl, 2-(3-fluorophenyl)ethyl, 2-(4-methylphenyl)ethyl, 2-(4-(trifluoromethyl)phenyl)ethyl, 2-(2-methoxyphenyl)ethyl, 2-(3-nitrophenyl)ethyl, 2-(2,4-dichlorophenyl)ethyl, 2-(3,5-dimethoxyphenyl)ethyl, 3-phenylpropyl, 3-(3-chlorophenyl)propyl, 3-(2-methylphenyl)propyl, 3-(4-methoxyphenyl)propyl, 3-(4-(trifluoromethyl)phenyl)propyl, 3-(2,4-dichlorophenyl)propyl, 4-phenylbutyl, 4-(4-chlorophenyl)butyl, 4-(2-methylphenyl)butyl, 4-(2,4-dichlorophenyl)butyl, 4-(2-methoxphenyl)butyl and 10-phenyldecyl.

The term “arcycloalkyl” is used to describe a group wherein the aryl group is attached to a cycloalkyl group, for example phenylcyclopentyl and the like.

The term “aralkenyl” is used to describe a group wherein the alkenyl chain can be branched or straight chain with the aryl portion, as defined hereinbefore, forming a terminal portion of the aralkenyl moiety, for example styryl (2-phenylvinyl), phenpropenyl and the like.

The term “aralkynyl” is used to describe a group wherein the alkynyl chain can be branched or straight chain with the aryl portion, as defined hereinbefore, forming a terminal portion of the aralkynyl moiety, for example 3-phenyl-1-propynyl and the like.

The term “aroxy” is used to describe an aryl group attached to a terminal oxygen atom. Typical aroxy groups include phenoxy, 3,4-dichlorophenoxy and the like.

The term “aroxyalkyl” is used to describe a group wherein an alkyl group is substituted with an aroxy group, for example pentafluorophenoxymethyl and the like.

The term “heteroaroxy” is used to describe an heteroaryl group attached to a terminal oxygen atom. Typical heteroaroxy groups include 4,6-dimethoxypyrimidin. 2-yloxy and the like.

The term “heteroaralyl” is used to describe a group wherein the alkyl chain can be branched or straight chain with the heteroaryl portion, as defined hereinbefore, forming a terminal portion of the heteroaralkyl moiety, for example 3-furylmethyl, thenyl, furfuryl and the like.

The term “heteroaralkenyl” is used to describe a group wherein the alkenyl chain can be branched or straight chain with the heteroaryl portion, as defined hereinbefore, forming a terminal portion of the heteroaralkenyl moiety, for example 3-(4-pyridyl)-1-propenyl.

The term “heteroaralkynyl” is used to describe a group wherein the alkynyl chain can be branched or straight chain with the heteroaryl portion, as defined hereinbefore, forming a terminal portion of the heteroaralkynyl moiety, for example 4-(2-thienyl)-1-butynyl.

The term “heterocyclyl” refers to a substituted or unsubstituted 5 or 6 membered saturated ring containing one, two or three heteroatoms, preferably one or two heteroatoms independently selected from oxygen, nitrogen and sulfur or to a bicyclic ring system containing up to 10 atoms including one heteroatom selected from oxygen, nitrogen and sulfur wherein the ring containing the heteroatom is saturated. Examples of heterocyclyls include, but are not limited to, tetrahydrofuryl, pyrrolidinyl, piperidinyl, tetrahydropyranyl, morpholinyl, piperazinyl, dioxolanyl, dioxanyl, indolinyl and 5-methyl-6-chromanyl.

The term “heterocyclylalkyl” is used to describe a group wherein the alkyl chain can be branched or straight chain with the heterocyclyl portion, as defined hereinabove, forming a terminal portion of the heterocyclylalkyl moiety, for example 3-piperidinylmethyl and the like.

The term “heterocyclylalkenyl” is used to describe a group wherein the alkenyl chain can be branched or straight chain with the heterocyclyl portion, as defined hereinbefore, forming a terminal portion of the heterocyclylalkenyl moiety, for example 2-morpholinyl-1-propenyl.

The term “heterocyclylalkynyl” is used to describe a group wherein the alkynyl chain can be branched or straight chain with the heterocyclyl portion, as defined hereinbefore, forming a terminal portion of the heterocyclylalkynyl moiety, for example 2-pyrrolidinyl-1-butynyl.

The term “carboxyalkyl” includes both branched and straight chain alkyl groups as defined hereinbefore attached to a carboxy (—COOH) group.

The term “carboxyalkenyl” includes both branched and straight chain alkenyl groups as defined hereinbefore attached to a carboxy group.

The term “carboxyalkynyl” includes both branched and straight chain alkynyl groups as defined hereinbefore attached to a carboxy group.

The term “carboxycycloalkyl” refers to a carboxy group attached to a cyclic aliphatic ring structure as defined hereinbefore.

The term “carboxycycloalkenyl” refers to a carboxy group attached to a cyclic aliphatic ring structure having 1 or 2 ethylenic bonds as defined hereinbefore.

The term “cycloalkylalkyl” refers to a cycloalkyl group as defined hereinbefore attached to an alkyl group, for example cyclopropylmethyl, cyclohexylethyl and the like.

The term “cycloalkylalkenyl” refers to a cycloalkyl group as defined hereinbefore attached to an alkenyl group, for example cyclohexylvinyl, cycloheptylallyl and the like.

The term “cycloalkylalkynyl” refers to a cycloalkyl group as defined hereinbefore attached to an alkynyl group, for example cyclopropylpropargyl, 4-cyclopentyl-2-butynyl and the like.

The term “cycloalkenylalkyl” refers to a cycloalkenyl group as defined hereinbefore attached to an alkyl group, for example 2-(cyclopenten-1-yl)ethyl and the like.

The term “cycloalkenylalkenyl” refers to a cycloalkenyl group as defined hereinbefore attached to an alkenyl group, for example 1-(cyclohexen-3-yl)allyl and the like.

The term “cycloalkenylalkynyl” refers to a cycloalkenyl group as defined hereinbefore attached to an alkynyl group, for example 1-(cyclohexen-3-yl)propargyl and the like.

The term “carboxycycloalkylalkyl” refers to a carboxy group attached to the cycloalkyl ring portion of a cycloalkylalkyl group as defined hereinbefore.

The term “carboxycycloalkylalkenyl” refers to a carboxy group attached to the cycloalkyl ring portion of a cycloalkylalkenyl group as defined hereinbefore.

The term “carboxycycloalkylalkynyl” refers to a carboxy group attached to the cycloalkyl ring portion of a cycloalkylalkynyl group as defined hereinbefore.

The term “carboxycycloalkenylalkyl” refers to a carboxy group attached to the cycloalkenyl ring portion of a cycloalkenylalkyl group as defined hereinbefore.

The term “carboxycycloalkenylalkenyl” refers to a carboxy group attached to the cycloalkenyl ring portion of a cycloalkenylalkenyl group as defined hereinbefore.

The term “carboxycycloalkenylalkynyl” refers to a carboxy group attached to the cycloalkenyl ring portion of a cycloalkenylalkynyl group as defined hereinbefore.

The term “alkoxy” includes both branched and straight chain alkyl groups attached to a terminal oxygen atom. Typical alkoxy groups include methoxy, ethoxy, n-propoxy, isopropoxy, tert-butoxy and the like.

The term “haloalkoxy” refers to an alkoxy group substituted with one or more halo groups, for example chloromethoxy, trifluoromethoxy, difluoromethoxy, perfluoroisobutoxy and the like.

The term “alkoxyalkoxyalkyl” refers to an alkyl group substituted with an alkoxy moiety which is in turn substituted with a second alkoxy moiety, for example methoxymethoxymethyl, isopropoxymethoxyethyl and the like.

The term “alkylthio” includes both branched and straight chain alkyl groups attached to a terminal sulfur atom, for example methylthio.

The term “haloalkylthio” refers to an alkylthio group substituted with one or more halo groups, for example trifluoromethylthio.

The term “alkoxyalkyl” refers to an alkyl group substituted with an alkoxy group, for example isopropoxymethyl.

The term “alkoxyalkenyl” refers to an alkenyl group substituted with an alkoxy group, for example 3-methoxyallyl.

The term “alkoxyalkynyl” refers to an alkynyl group substituted with an alkoxy group, for example 3-methoxypropargyl.

The term “alkoxycarbonylalkyl” refers to a straight chain or branched alkyl substituted with an alkoxycarbonyl, for example ethoxycarbonylmethyl, 2-(methoxycarbonyl)propyl and the like.

The term “alkoxycarbonylalkenyl” refers to a straight chain or branched alkenyl as defined hereinbefore substituted with an alkoxycarbonyl, for example 4-(ethoxycarbonyl)-2-butenyl and the like.

The term “alkoxycarbonylalkynyl” refers to a straight chain or branched alkynyl as defined hereinbefore substituted with an alkoxycarbonyl, for example 4-(ethoxycarbonyl)-2-butynyl and the like.

The term “haloalkoxyalkyl” refers to a straight chain or branched alkyl as defined hereinbefore substituted with a haloalkoxy, for example 2-chloroethoxymethyl, trifluoromethoxymethyl and the like.

The term “haloalkoxyalkenyl” refers to a straight chain or branched alkenyl as defined hereinbefore substituted with a haloalkoxy, for example 4-(chloromethoxy)-2-butenyl and the like.

The term “haloalkoxyalkynyl” refers to a straight chain or branched alkynyl as defined hereinbefore substituted with a haloalkoxy, for example 4-(2-fluoroethoxy)-2-butynyl and the like.

The term “alkylthioalkyl” refers to a straight chain or branched alkyl as defined hereinbefore substituted with an alkylthio group, for example methylthiomethyl, 3-(isobutylthio)heptyl and the like.

The term “alkylthioalkenyl” refers to a straight chain or branched alkenyl as defined hereinbefore substituted with an alkylthio group, for example 4-(methylthio)-2-butenyl and the like.

The term “alkylthioalkynyl” refers to a straight chain or branched alkynyl as defined hereinbefore substituted with an alkylthio group, for example. 4-(ethylthio)-2-butynyl and the like.

The term “haloalkylthioalkyl” refers to a straight chain or branched alkyl as defined hereinbefore substituted with an haloalkylthio group, for example 2-chloroethylthiomethyl, trifluoromethylthiomethyl and the like.

The term “haloalkylthioalkenyl” refers to a straight chain or branched alkenyl as defined hereinbefore substituted with an haloalkylthio group, for example 4-(chloromethylthio)-2-butenyl and the like.

The term “haloalkylthioalkynyl” refers to a straight chain or branched alkynyl as defined hereinbefore substituted with an haloalkylthio group, for example 4-(2-fluoroethylthio)-2-butynyl and the like.

The term “dialkoxyphosphorylalkyl” refers to two straight chain or branched alkcoxy groups as defined hereinbefore attached to a pentavalent phosphorous atom, containing an oxo substituent, which is in turn attached to an alkyl, for example diethoxyphosphorylmethyl.

The term “oligomer” refers to a low-molecular weight polymer, whose number average molecular weight is typically less than about 5000 g/mol, and whose degree of polymerization (average number of monomer units per chain) is greater than one and typically equal to or less than about 50.

In Schemes 1-20 hereinafter showing how to synthesize compounds of this invention and Tables 1-10 hereinafter listing various representative compounds of this invention, the following abbreviations may be present: Me for methyl, Et for ethyl, ⁱPr or ⁱPr for isopropyl, n-Bu for n-butyl, t-Bu for tert-butyl, Ac for acetyl, Ph for phenyl, 4Cl-Ph or (4Cl)Ph for 4-chlorophenyl, 4Me-Ph or (4Me)Ph for 4-methylphenyl, (p-CH₃O)Ph for p-methoxyphenyl, (p-NO₂)Ph for p-nitrophenyl, 4Br-Ph or (4Br)Ph for 4-bromophenyl, 2-CF3-Ph or (2CF₃)Ph for 2-trifluoromethylphenyl, Bn for benzyl and the like.

The compounds of Formula I of this invention and the intermediates used in the synthesis of the compounds of this invention can be prepared according to the following methods. Method A can be used when preparing compounds of Formula I-A [compound of Formula I where R¹ equals C(=G³⁰)-(G³¹)ₜ(X³)_{d}Z³] as shown below in Scheme 1:

Method A:

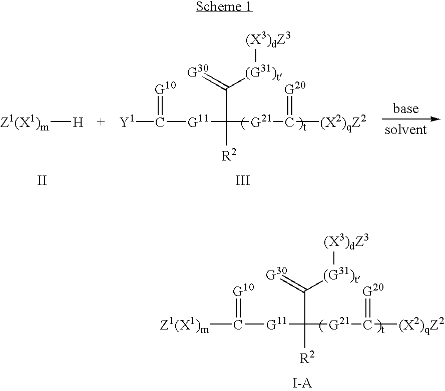

where Z¹(X¹)ₘ—H, Z²(X²)_{q}, Z³(X³)_{d}, Z¹(X¹)ₘ, G¹⁰, G¹¹, G²⁰, G²¹, G³⁰, G³¹, and R² are as defined previously for compound of Formula I-A and t=0 or 1, t′=0 or 1, q=C or 1, d=0 or 1, and Y¹=halogen such as chlorine.

In a typical preparation, according to Method A, of a compound of Formula I-A [compound of Formula I where R¹ equals C(=G³⁰)-(G³¹)_{t′}(X³)_{d}Z³], a compound of Formula II is reacted with a compound of Formula III in a suitable solvent in the presence of a suitable base. Suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula II. The compounds of Formula II are generally commercially available or can be prepared according to known procedures.

The compounds of Formula III of Scheme 1 are prepared as shown in Scheme 2.

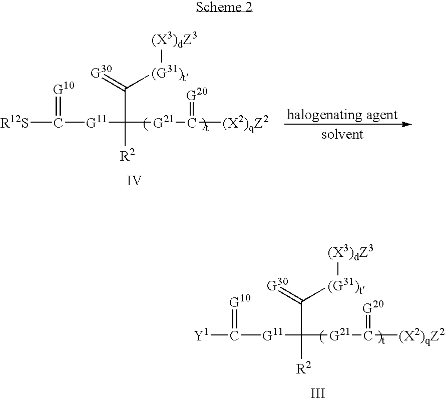

where Z²(X²)_{q}, Z³(X³)_{d}, G¹⁰, G¹¹, G²⁰, G²¹, G³⁰, G³¹, and R² are as defined previously for compound of Formula I-A and t=0 or 1, t′=0 or 1, q=0 or 1, d=0 or 1, R¹² is an alkyl, aralkyl, or aryl group, and Y¹=halogen such as chlorine.

In a typical preparation of a compound of Formula III, a compound of Formula IV is treated with a suitable halogenating agent in a suitable solvent, where the suitable halogenating agents include chlorine gas, thionyl chloride, and sulfuryl chloride, however, the preferred halogenating agent is sulfuryl chloride. Suitable solvents for use in the above process include, but are not limited to, hexanes, chlorinated solvents such as methylene chloride, dichloroethane, chloroform, carbon tetrachloride, and the like, however, the reactions are normally run neat. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired.

The compounds of Formula IV of Scheme 2 are prepared as shown in Scheme 3:

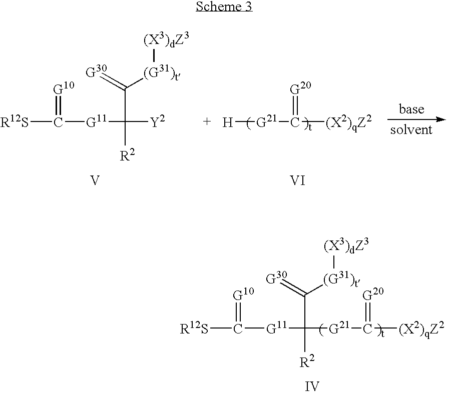

where Z²(X²)_{q}[(C=G²⁰)-G²¹]ₜ—H, Z²(X²)_{q}, Z³(X³)_{d}, G¹⁰, G¹¹, G²⁰, G²¹, G³⁰, G³¹, and R² are as defined previously for compound of Formula I and t=0 or 1, t′=0 or 1, q=0 or 1, d=0 or 1, R¹² is an alkyl, aralkyl, or aryl group, and Y²=halogen such as chlorine.

In a typical preparation of a compound of Formula IV, a compound of Formula V is reacted with a compound of Formula VI in a suitable solvent in the presence of a suitable base. Suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula VI. The compounds of Formula VI are generally commercially available or can be prepared according to known procedures.

The compounds of Formula V of Scheme 3 are prepared as shown in Scheme 4:

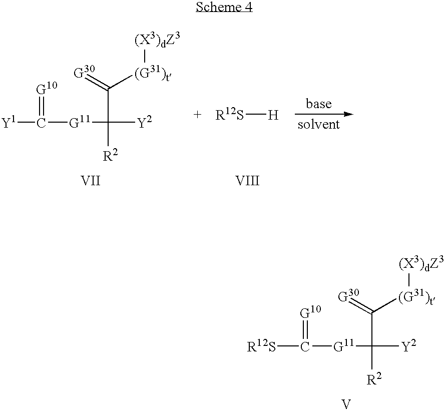

where Z³(X³)_{d}, G¹⁰, G¹¹, G³⁰, G³¹, and R² are as defined previously for compound of Formula I-A and t′=0 or 1, d=0 or 1, R¹²=alkyl, aralkyl, or aryl, and Y¹ and Y²=halogen such as chlorine, bromine, or iodine.

In a typical preparation of a compound of Formula V, a compound of Formula VII is reacted with a compound of Formula VIII in a suitable solvent in the presence of a suitable base. Suitable solvents for use in the above process include, but are not limited to, ethers such as tetrahydrofuran THF), glyme, diethyl ether, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used, however, the preferred solvent is diethyl ether. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used, however, the preferred base is sodium hydride. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula VIII. The compounds of Formula VIII are generally commercially available or can be prepared according to known procedures. Conversion of Y2 from Cl to Br or Cl to I in compound of Formula V can be prepared according to literature procedures. A general description of the synthesis of halogen exchange (Finkelstein reaction) is described in March, J. Advanced Organic Chemistry, 4th ed.; Wiley and Sons: New York, 1992; pp 430-431.

The compounds of Formula VII of Scheme 4 are prepared as shown in Scheme 5:

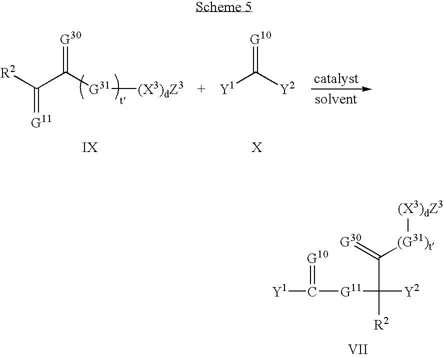

where Z³(X³)_{d}, G¹⁰, G¹¹, G³⁰, G³¹, and R² are as defined previously for compound of Formula I-A, t′=0 or 1, d=0 or 1, and Y¹ and Y²=halogen such as chlorine, bromine, or iodine.

In a typical preparation of a compound of Formula VII, a compound of Formula IX is reacted with a compound of Formula X (or a suitable precursor of compound of Formula X) in a suitable solvent in the presence of a suitable base. Suitable solvents for use in the above process include, but are not limited to, ethers such as tetrahydrofuran (THF), glyme, diethyl ether, dioxane and the like; aromatic solvents such as benzene and toluene; acetonitrile; chlorinated solvents such as methylene chloride (CH₂Cl₂), carbon tetrachloride (CCl₄) or chloroform (CHCl₃). If desired, mixtures of these solvents may be used. The preferred solvent is dependent upon the substrates employed and is selected according to the properties of the substrates. Suitable catalysts for use in the above process include, but are not limited to, pyridine, thioureas and ureas such as tetra-n-butylurea, phosphoramides such as hexamethylphosphotriamide, substituted amides such as dimethylformamide, quaternary ammonium halides such as tetrabutyl or tributylbenzyl ammonium chloride, arylamines such as N,N,-dimethylaminopyridine, N,N-dimethylaniline, tertiary phosphines such as trioctyl phosphine, and alkali metal or alkaline earth metal halides such as cesium or potassium chloride which are used in conjunction with a sequestering agent such as a crown ether (18-crown-6). If desired, mixtures of these catalysts may be used, however, the preferred catalyst is pyridine. Compound of Formula IX may in some cases exist in a polymeric form. If so, the monomeric form can be achieved via, known procedures, one being through thermal depolymerization. Compound of Formula X when G¹⁰=O and Y¹ and Y²=Cl is phosgene, C(═O)Cl₂. However, other forms of phosgene, phosgene equivalents, can be utilized such as trichloromethyl chloroformate (compound of Formula X in which G¹⁰=O, Y¹=Cl, and Y²=OCCl₃) or di(trichloromethyl)carbonate (compound of Formula X in which G¹⁰=O and Y¹ and Y²=OCCl₃). The above process may be carried out at temperatures between about −78° C. and about 100° C. Preferably, the reaction is carried out between 0° C. and about 100° C. The above process to produce compounds of the present invention is preferably carried out at about atmospheric pressure although higher or lower pressures can be used if desired. Substantially equimolar amounts of reactants are preferably used although higher or lower amounts can be used if desired. The catalyst is normally used in lower amounts than that of both compounds of Formula IX and X. The compounds of Formula IX and X are generally commercially available or can be prepared according to known procedures.

The compounds of Formula I-A [compound of Formula I where R¹ equals C(=G³⁰)-(G³¹)_{t′}(X³)_{d}Z³] can be prepared according to Method B as shown in Scheme 6:

Method B:

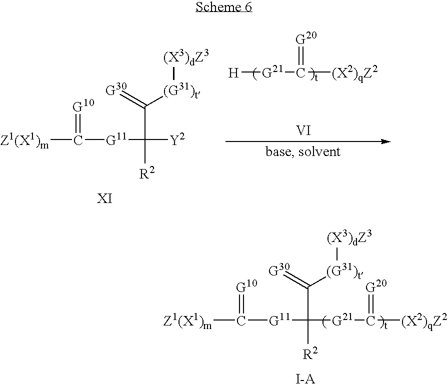

where Z¹(X¹)ₘ, Z²(X²)_{q}, Z³(X³)_{d}, G¹⁰, G¹¹, G²⁰, G²¹, G³⁰, G³¹, and R² are as defined previously for compound of Formula I-A and m=0 or 1, t=0 or 1, q=0 or 1, t′=0 or 1, d=0 or 1, and Y²=halogen such as chlorine, bromine, or iodine.

In a typical preparation, according to Method B, Scheme 6, of a compound of Formula I-A [compound of Formula I where R¹ equals C(=G³⁰)-(G³¹)_{t′}(X³)_{d}Z³], a compound of Formula XI is reacted with a compound of Formula VI in a suitable solvent in the presence of a suitable base. Suitable solvents for use in the above process include, but are not limited to, ethers such as tetrahydrofuran (THF), glyme, diethyl ether, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula VI. The compounds of Formula VI are generally commercially available or can be prepared according to known procedures.

The compounds of Formula XI of Scheme 6 are prepared as shown in Scheme 7:

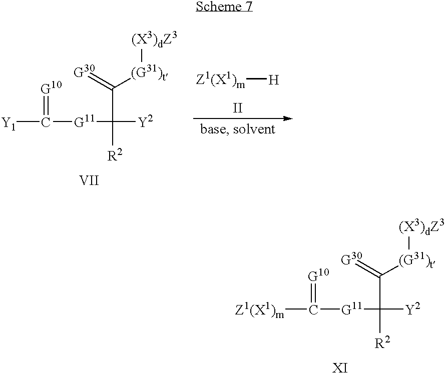

where Z¹(X¹)ₘ—H, Z³(X³)_{d}, Z¹(X¹)ₘ, G¹⁰, G¹¹, G³⁰, G³¹, and R² are as defined previously for compound of Formula I-A and m=0 or 1, t′=0 or 1, d=0 or 1, and Y¹ and Y²=halogen such as chlorine.

In a typical preparation of a compound of Formula XI, a compound of Formula II is reacted with a compound of Formula VII in a suitable solvent in the presence of a suitable base. Suitable solvents for use in the above process include, but are not limited to, ethers such as tetrahydrofuran (THF), glyme, diethyl ether, and the like; dimethylformamide (DMF); dimethylsulfoxide (DMSO); acetonitrile; chlorinated solvents such as methylene chloride (CH₂CL) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula II. The compounds of Formula II are generally commercially available or can be prepared according to known procedures. Conversion of Y² from Cl to Br or Cl to I in compound of Formula XI can be prepared according to literature procedures. A general description of the synthesis of halogen exchange (Finkelstein reaction) is described in March, J. Advanced Organiic Chemistry, 4^{th} ed.; Wiley and Sons: New York, 1992; pp 430-431.

The compounds of Formula I-A [compound of Formula I where R¹ equals C(=G³⁰)-(G³¹)_{t′}(X³)_{d}Z³] can be prepared according to Method C as shown in Scheme 8:

Method C:

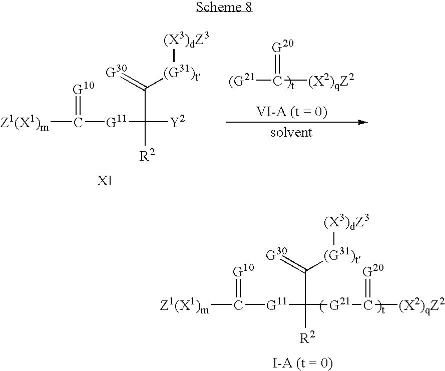

where Y²=halogen such of as iodine, bromine, or chlorine, Z¹(X¹)ₘ, Z³(X³)_{d}, G¹⁰, G¹¹, G³⁰, G³¹, and R² are as defined previously for compound of Formula I-A and m=0 or 1, t=0, q=0 or 1, t′=0 or 1, and d=0 or 1 where Z²(X²)_{q}═NR³R⁴, NR³R⁴R⁵, or {(NR³R⁴R⁵)M} and when q=0, in compound of Formula VI-A, Z² can be a tertiary amine where in compound of Formula I-A, Z² is a quaternary amine salt, and when q=1, in compound of Formula VI-A, X²Z² can be a tertiary amine where in compound of Formula I-A, X²Z² is a quaternary amine salt.

In a typical preparation of a compound of Formula I-A [compound of Formula I where R¹ equals C(=G³⁰)-(G³¹)_{t′}(X³)_{d}Z³], according to Method C, a compound of Formula XI is reacted with a compound of Formula VI-A in a suitable solvent. Suitable solvents for use in the above process include, but are not limited to, ethers such as tetrahydrofuran (THF), glyme, diethyl ether, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. The above process may be carried out at temperatures between about −78° C. and about 200° C. Preferably, the reaction is carried out between 0° C. and about 100° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of compound of Formula XI is used per equivalent of starting material of compound of Formula VI-A. The compounds of Formula VI-A are generally commercially available or can be prepared according to known procedures. The compounds of Formula XI can be prepared by the same process as that of Scheme 7.

The compounds of Formula I-B, I-C, and I-A [compounds of Formula I where R¹ equals C(=G³⁰)-(G³¹)_{t′}-H, C(=G³⁰)-Y¹, and C(=G³⁰)-(G³¹)_{t′}(X³)_{d}Z³, respectively] can be prepared according to Method D as shown below in Scheme 9:

Method D:

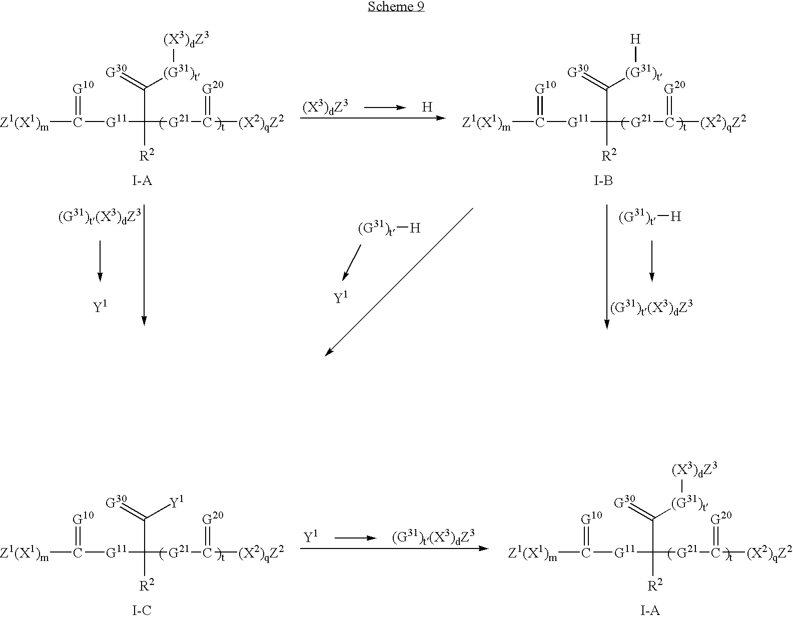

where Z¹(X¹)ₘ, Z²(X²)_{q}, Z³(X³)_{d}, G¹⁰, G¹¹, G²⁰, G²¹, G³⁰, G³¹, and R² are as defined above for compound of Formula I-A and m 0 or 1, t=0 or 1, t′=0 or 1, q=0 or 1, d=0 or 1, and Y¹=halogen such as chlorine.

In a typical preparation of a compound of Formula I-B [compound of Formula I where R¹ equals C(=G³⁰)-(G³¹)_{t′}-H], a compound of Formula I-A [compound of Formula I where R¹ equals C(=G³⁰)-(G³¹)_{t′}(X³)_{d}Z³] is treated with a suitable solvent under suitable reaction conditions which can successfully transform (G³¹)_{t′}(X³)_{d}Z³ to (G³¹)_{t′}-H via methods known to one skilled in the art. For example, if d=0, Z³=benzyl (Bn), t′=1, and G³⁰ and G³¹=oxygen, then typical reaction conditions for the transformation of C(=G³⁰)-G³¹Z³ (CO₂Bn) to C(=G³⁰)-G³¹H(CO₂H) would involve the following: suitable solvents include, but are not limited to, ethers such as tetrahydrofuran (THF), glyme, and the like; esters such as ethyl acetate; acetonitrile; alcohols such as methanol or ethanol; chlorinated solvents such as methylene chloride (CH₂Cl₂) or chloroform (CHCl₃). If desired, mixtures of these solvents may be used, however, the preferred solvent is ethyl acetate. Suitable catalysts in the presence of at least one equivalent of hydrogen include, palladium, platinum, nickel, rhodium, iridium and ruthenium. The catalysts are normally adsorbed or admixed on an inert support material which includes carbon, alumina, calcium sulfate, or barium sulfate, however, the preferred catalyst and support is palladium on carbon. The above process to produce compounds of the present invention is preferably carried out at about atmospheric pressure although higher or lower pressures can be used if desired. Substantially equimolar amounts of reactants are preferably used although higher or lower amounts can be used if desired. One skilled in the art would recognize that a hydrogenation reaction to remove a benzyl group as described above would only be utilized when all other functional groups present within compound of Formula I-A are deemed compatible with the reaction conditions. See Greene, T. W.; Wuts, P. G. M. Protective Groups in Organic Synthesis; 2nd ed.; Wiley and Sons: New York, 1991; pp 227-265 for additional suitable reaction conditions and appropriate C(=G³⁰)-(G³¹)_{t′}(X³)_{d}Z³ chemical moieties for the transformation of (G³¹)_{t′}(X³)_{d}Z³ to (G³¹)_{t′}-H.

In a typical preparation of a compound of Formula I-C [compound of Formula I where R¹ equals C(=G³⁰)-Y¹], a compound of Formula I-B [compound of Formula I where R¹ equals C(=G³⁰)-(G³¹)_{t′}-H] is treated with a suitable halo-de-hydroxylation reagent in a suitable solvent, where suitable halo-de-hydroxylation reagents include, but are not limited to, thionyl chloride, oxalyl chloride, oxalyl bromide, triphenyl phosphine in carbon tetrachloride, phosphorus trichloride, and phosphorus pentachloride, however, the preferred halo-de-hydroxylation reagent is thionyl chloride. Suitable solvents for use in the above process include, but are not limited to, hexanes, ethers such as tetrahydrofuran, glyme, and the like; chlorinated solvents such as methylene chloride, dichloroethane, chloroform, carbon tetrachloride, and the like, however, the reactions are normally run neat with a catalytic amount of dimethylformamide present. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Compounds of Formula I-C [compound of Formula I where R¹ equals C(=G³⁰)-Y¹] can also be synthesized directly from compounds of Formula I-A [compound of Formula I where R¹ equals C(=G³⁰)-(G³¹)_{t′}(X³)_{d}Z³]. In a typical preparation of a compound of Formula I-C, a compound of Formula I-A is treated with a suitable solvent in the presence of suitable reaction conditions which can successfully transform C(=G³⁰)-(G³¹)_{t′}(Y³)_{d}Z³ to C(=G³⁰)-Y¹ via methods known to one skilled in the art. For example, if d=0, t′=1, and Z³=trimethylsilyl and G³⁰ and G³¹=oxygen, then typical reaction conditions for the transformation of C(=G³⁰)-G³¹Z³ (CO₂SiMe₃) to C(=G³⁰)-Y¹ [C(═O)—Cl] would involve treatment of the compound of Formula I-A in which C(=G³⁰)-G³¹Z³ is CO₂SiMe₃ with oxalyl chloride in a suitable solvent such as THF at a temperature ranging from −78° C. and about 100° C. See Larock, R. C. Comprehensive Organic Transformations, 2nd ed, New York, 1999; p 1968 as well as Greene, T. W.; Wuts, P. G. M. Protective Groups in Organic Synthesis; 2nd ed.; Wiley and Sons: New York, 1991; pp 261-262 for additional suitable reaction conditions for the transformation of C(=G³⁰)-(G³¹)_{t′}(X³)_{d}Z³ to C(=G³⁰)-Y¹. One skilled in the art would recognize that a halogenation reaction to convert a silyl ester to an acid chloride as described above would only be utilized when all other functional groups present within compounds of Formula I-A are deemed compatible with the reaction conditions. Compound of Formula I-C in which Y¹ is chlorine can be interconverted to other halo derivatives via reaction conditions listed in Larock, R. C. Comprehensive Organic Transformations, 2nd ed.; Wiley and Sons: New York, 1999; pp 1950-1951.

In a typical preparation of a compound of Formula I-A [compound of Formula I where R¹ equals C(=G³⁰)-(G³¹)_{t′}(X³)_{d}Z³] from a compound of Formula I-C [compound of Formula I where R¹ equals C(=G³⁰)-Y¹], a compound of Formula I-C is reacted with Z³(X³)_{d}(G³′)_{t′}-H in a suitable solvent in the presence of a suitable base. Suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula I-C. Suitable reaction conditions for the conversion of C(=G³⁰)-Y¹ to C(=G³⁰)-(G³¹)_{t′}(X³)_{d}Z³ can be found in Larock, R. C. Comprehensive Organic Transformations, 2nd ed.; Wiley and Sons: New York, 1999; pp 1952-1954.

Additionally, a compound of Formula I-A [compound of Formula I where R¹ equals C(=G³⁰)-(G³¹)_{t′}(X³)_{d}Z³] can be prepared via the reaction of a compound of Formula I-B [compound of Formula I where R¹ equals C(=G³⁰)-(G³¹)_{t′}-H] in a suitable solvent with Z³(X³)_{d}—H and a suitable coupling reagent. Suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried uut at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula I-B. Suitable coupling reagents for use in the above process include, but are not limited to, lipases, diazo compounds, anhydrides, acid chlorides, carbodiimides, and carbodiimidazoles. Suitable reaction conditions for the conversion of C(=G³⁰)-(G³¹)_{t′}-H to C(=G³⁰)-(G³¹)_{t′}(X³)_{d}Z³ can be found in Larock, R. C. Comprehensive Organic Transformations, 2nd ed.; Wiley and Sons: New York, 1999; pp 1932-1949.

Alternatively, transformation of a compound of Formula I-B [compound of Formula I where R¹ equals C(=G³⁰)-(G³′)_{t′}-H] to a compound of Formula I-A [compound of Formula I where R¹ equals C(=G³⁰)-(G³¹)_{t′}(X³)_{d}Z³] can also be accomplished by reaction of a compound of Formula I-B in a suitable solvent with a suitable base in the presence of a suitable commercially available halide, such as an alkyl halide. Suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. Suitable halides include, but are not limited to, alkyl halides such as benzyl chloride, benzyl bromide, methyl iodide, and ethyl iodide. The preferred halide is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula I-B. Suitable reaction conditions for the conversion of C(=G³⁰)-(G³¹)_{t′}-H to C(=G³⁰)-(G³¹)_{t′}(X³)_{d}Z³ can be found in Larock, R. C. Comprehensiue Organic Transformations, 2^{nd} ed.; Wiley and Sons: New York, 1999; pp 1938-1940.

The compounds of Formula I-D of this invention and the intermediates used in the synthesis of the compounds of this invention can be prepared according to the following methods. Method E can be used when preparing compounds of Formula I-D as shown below in Scheme 10:

Method E:

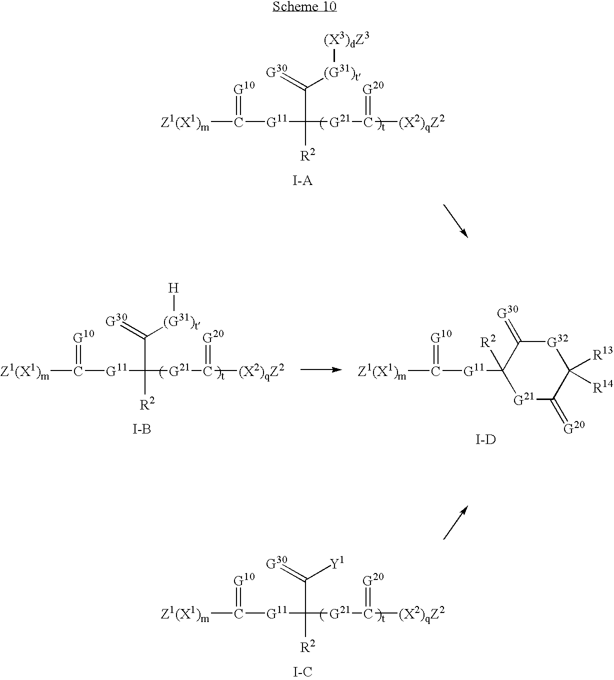

where Z¹(X¹)ₘ, Z³(X³)_{d}, G¹⁰, G¹¹, G²⁰, G²¹, G³⁰, G³¹, and R² are as defined above for compound of Formula I-A, m=0 or 1, Y¹=halogen such as chlorine, and q=0 and Z²=CR¹³R¹⁴G³², where G³² is an oxygen atom, a sulfur atom or NR³, and R¹³ and R¹⁴ are independently defined as for R².

In a typical preparation of a compound of Formula I-D, a compound of Formula I-A [compound of Formula I where R¹ equals C(=G³⁰)-(G³¹)_{t′}(X³)_{d}Z³], where the C(=G³⁰)-(G³¹)_{t′}(X³)_{d}Z³ moiety is reacted intramolecularly with the (X²)_{q}Z² moiety (where q=0 and Z²=CR¹³R¹⁴G³²) in a suitable solvent in the presence of a suitable base to afford cyclic [—C(=G³⁰)-G³²-CR¹³R¹⁴C(=G²⁰)G²¹C(R²)—]. Suitable solvents include, but are not limited to, ethers such as tetrahydrofuran (THF), glyme, and the like; esters such as ethyl acetate; acetonitrile; alcohols such as methanol or ethanol; chlorinated solvents such as methylene chloride (CH₂Cl₂) or chloroform (CHCl₃). The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula I-A.

A compound of Formula I-D can also be prepared via the reaction of a compound of Formula I-B [compound of Formula I where R¹ equals C(=G³⁰)-(G³¹)_{t′}-H], where C(=G³⁰)-(G³¹)_{t′}-H is reacted intramolecularly with (X²)_{q}Z², where Z²═CR¹³R¹⁴G³², in a suitable solvent in the presence of a suitable base along with a suitable coupling reagent. Suitable reaction conditions would involve the following: suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula I-B. Suitable coupling reagents for use in the above process include, but are not limited to, lipases, diazo compounds, anhydrides, acid chlorides, carbodiimides, and carbodiimidazoles. Suitable reaction conditions for the conversion of H-(G³¹)_{t′}-(G³⁰=)C—C(R²)-(G²¹[G²⁰=]C)ₜ(X²)_{q}Z² where Z²=CR¹³R¹⁴G³², to cyclic [—C(=G³⁰)-G³²-CR¹³R¹⁴C(=G²⁰)G²¹C(R²)—] can be found in Larock, R. C. Comprehensive Organic Transformations, 2nd ed.; Wiley and Sons: New York, 1999; pp 1932-1949.

In a typical preparation of a compound of Formula I-D, a compound of Formula I-C [compound of Formula I where R¹ equals C(=G³⁰)-Y¹] where the C(=G³⁰)Y¹ moiety is reacted intramolecularly with the (X²)_{q}Z² moiety (where Z²=CR¹³R¹⁴G³²) in a suitable solvent in the presence of a suitable base. Suitable reaction conditions would involve the following: suitable solvents for use in the above process include, but are not limited to, ethers such as tetrahydrofuran (THF), glyme, and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used. The preferred solvent is dependent upon the substrates employed and is selected according to the properties of the substrates. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used. The preferred base is dependent upon the substrates employed and is selected according to the properties of the substrates. The above process may be carried out at temperatures between about −78° C. and about 100° C. Preferably, the reaction is carried out between 0° C. and about 50° C. The above process to produce compounds of the present invention is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula I-C. Suitable reaction conditions for the conversion of Y¹-(G³⁰=)C—C(R²)-(G²¹[G²⁰=]C)ₜ(X²)_{q}Z², where Z² CR¹³R¹⁴G³², to cyclic [-C(=G³⁰)-G³²CR¹³R¹⁴C(=G²⁰)G²⁰C(R²)—] can be found in Larock, R. C. Comprehensive Organic Transformations, 2nd ed.; Wiley and Sons: New York, 1999; pp 1952-1954.

Application of Method A (Scheme 5) as described previously for the synthesis of compound of Formula VII to the synthesis of compound of Formula XIV is described below in Scheme 11. Compound of Formula XII (compound of Formula IX where G¹¹ and G³⁰=O) is reacted with compound of Formula XIII (compound of Formula X where G¹⁰=O) to afford compound of Formula XIV (compound of Formula VII where G¹⁰, G¹¹, and G³⁰=O):

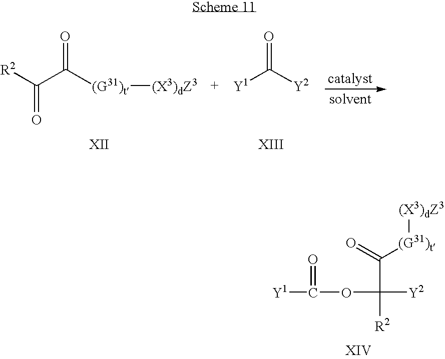

where Z³(X³)_{d}, d, t′, G³¹, and R² are as defined previously for compound of Formula I-A, Y¹ and Y²=halogen such as chlorine, bromine, or iodine.

Suitable solvents for use in the above process include, but are not limited to, ethers such as tetrahydrofuran (THF), glyme, diethyl ether, dioxane and the like; acetonitrile; chlorinated solvents such as methylene chloride (CH₂Cl₂), carbon tetrachloride (CCl₄) or chloroform (CHCl₃). If desired, mixtures of these solvents may be used. The preferred solvent is dependent upon the substrates employed and is selected according to the properties of the substrates. For example, in the reaction of compound of Formula XII, where t′=1, G³¹=O, d=0, R²=H, and Z³=benzyl (Bn), with compound of Formula XIII, where Y¹ and Y²=Cl, to afford compound of Formula XIV where t′=1, G³¹=O, d=0, R²=H, Z³=benzyl (Bn), and Y¹ and Y²=Cl, then the preferred solvent is carbon tetrachloride. However, in the reaction of compound of Formula XII, where t′=1, G³¹=O, d=0, R²=H, and Z³=ethyl (Et), with compound of Formula XIII, where Y¹ and Y²=Cl, to afford compound of Formula XIV, where t′=1, G³¹=O, d=0, R²=H, Z³=ethyl (Et), and Y¹ and Y²=Cl, then the preferred solvent is tetrahydrofuran. Suitable catalysts for use in the above process include, but are not limited to, pyridine, thioureas and ureas such as tetra-n-butylurea, phosphoramides such as hexamethylphosphotriamide, substituted amides such as dimethylformamide, quaternary ammonium halides such as tetrabutyl or tributylbenzyl ammonium chloride, arylamines such as N,N,-dimethylaminopyridine, N,N-dimethylaniline, tertiary phosphines such as trioctyl phosphine, and alkali metal or alkaline earth metal halides such as cesium or potassium chloride which are used in conjunction with a sequestering agent such as a crown ether (18-crown-6). If desired, mixtures of these catalysts may be used, however, the preferred catalyst is pyridine. Compound of Formula XII may in some cases exist in a polymeric form. If so, the monomeric form can be achieved via known procedures, one being through thermal depolymerization. Compound of Formula XIII in which Y¹ and Y²=Cl is phosgene, C(═O)Cl₂. However, other forms of phosgene, phosgene equivalents, can be utilized such as trichloromethyl chloroformate (compound of Formula XIII in which Y¹=Cl, and Y²=OCCl₃) or di(trichloromethyl)carbonate (compound of Formula XIII in which Y¹ and Y²=OCCl₃). The above process may be carried out at temperatures between about −78° C. and about 100° C. Preferably, the reaction is carried out between 0° C. and about 100° C. The above process to produce compounds of the present invention is preferably carried out at about atmospheric pressure although higher or lower pressures can be used if desired. Substantially equimolar amounts of reactants are preferably used although higher or lower amounts can be used if desired. The catalyst is normally used in lower amounts than that of both compounds of Formula XII and XIII. The compounds of Formula XII and XIII are generally commercially available or can be prepared according to known procedures.

Application of Method A (Scheme 4) as described previously for the synthesis of compound of Formula V to the synthesis of compound of Formula XVI is described below in Scheme 12. Compound of Formula XV (compound of Formula VIII where R¹²=Et and R¹²S—H is taken together to equal Et-S-L) is reacted with compound of Formula XIV (compound of Formula VII where G¹⁰, G¹¹, and G³⁰O) to afford compound of Formula XVI (compound of Formula V where G¹⁰, G¹¹, and G³⁰O, and R¹²=Et):

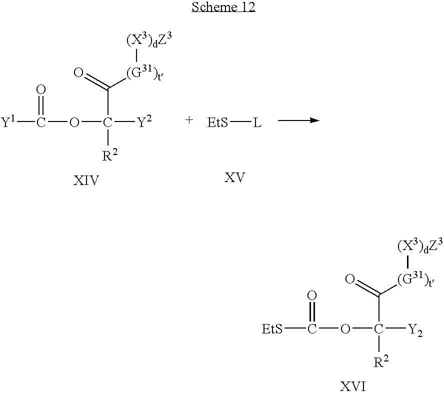

where Z³(X³)_{d}, d, t′, G³¹ and R² are as defined previously for compound of Formula I-A, L=metal cation such as Na or K, and Y¹ and Y²=halogen such as chlorine, bromine, or iodine.

Suitable solvents for use in the above process include, but are not limited to, ethers such as tetrahydrofuran (THF), glyme, diethyl ether and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used, however, the preferred solvent is diethyl ether. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; or pyridine. If desired, mixtures of these bases may be used, however, the preferred base is sodium hydride. The above process may be carried out at temperatures between about −78° C. and about 100° C. Preferably, the reaction is carried out between 0° C. and about 50° C. Preparation of the compounds of the present invention by the above process is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula XV except when L=Na or K, then no base is required. The compounds of Formula XV are generally commercially available or can be prepared according to known procedures. For example, R¹²S-L=EtS-Na, is commercially available. Conversion of Y² from Cl to Br or Cl to I in compound of Formula XVI can be prepared according to literature procedures. A general description of the synthesis of halogen exchange (Finkelstein reaction) is described in March, J. Advanced Organic Chemistry, 4th ed.; Wiley and Sons: New York, 1992; pp 430-431. Also, see synthesis Example 8 for conversion of Y² from Cl to I in compound of Formula XVI.

Application of Method A (Scheme 3) as described previously for the synthesis of compound of Formula IV to the synthesis of compound of Formula XVII is described below in Scheme 13. Compound of Formula XVI (compound of Formula V where R¹²=Et and G¹⁰, G¹¹, and G³⁰O) is reacted with compound of Formula VI (where t=1 and q=0 or 1) to afford compound of Formula XVII (compound of Formula IV in which t=1, G¹⁰, G¹¹, and G³⁰O, R¹²=Et, and q=0 or 1):

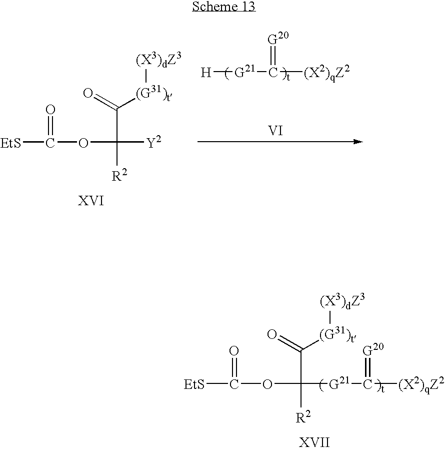

where Z²(X²)_{q}, Z³(X³)_{d}, d, t′, G²⁰, G²¹, G³¹, and R² are as defined previously for compound of Formula -A, and Y²=halogen such as chlorine, bromine, or iodine

Suitable solvents for use in the above process include, but are not limited to, ethers such as tetrahydrofuran (THF), glyme, diethyl ether and the like; dimethylformamide (DMF); dimethylsulfoxide (DMSO); acetonitrile; chlorinated solvents such as methylene chloride (CH₂Cl₂) or chloroform (CHCl₃). If desired, mixtures of these solvents may be used, however, the preferred solvent is THF. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or ciisopropylethylamine; an alkali metal carbonate such as sodium or potassium carbonate; or pyridine. If desired, mixtures of these bases may be used, however, the preferred base is diisopropylethylamine. The above process may be carried out at temperatures between about −78° C. and about 100° C. Preferably, the reaction is carried out between 0° C. and about 50° C. Preparation of the compounds of the present invention by the above process is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula VI. The compounds of Formula VI are generally commercially available or can be prepared according to known procedures.

Application of Method A (Scheme 2) as described previously for the synthesis of compound of Formula III to the synthesis of compound of Formula XVIII is described below in Scheme 14. Compound of Formula XVII (compound of Formula IV where R¹²=Et, G¹⁰, G¹¹, and G³⁰=O, and q=0 or 1) is reacted with a suitable halogenating agent to afford compound of Formula XVIII (compound of Formula III where G¹⁰, G¹¹, and G³⁰=O. Y¹=Cl, and q=0 or 1):

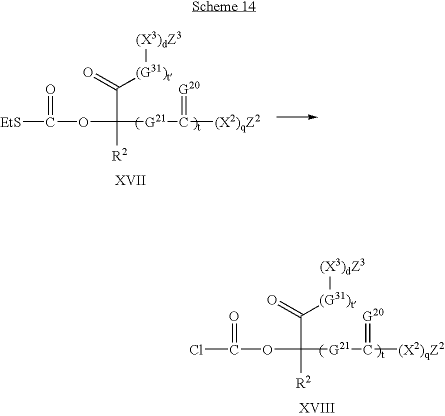

where Z²(X²)_{q}, Z³(X³)_{d}, d, t′, G²⁰, G²¹, G³¹, and R² are as defined previously for compound of Formula I-A and t=1.

Suitable halogenating agents include chlorine gas, thionyl chloride, and sulfuryl chloride, however, the preferred halogenating agent is sulfuryl chloride. Suitable solvents for use in the above process include, but are not limited to, hexanes, chlorinated solvents such as methylene chloride, dichloroethane, chloroform, carbon tetrachloride and the like, however, the reactions are normally run neat. The above process may be carried out at temperatures between about −78° C. and about 100° C. Preferably, the reaction is carried out between 0° C. and about 50° C. Preparation of the compounds of the present invention by the above process is preferably carried out at about atmospheric pressure although higher or lower pressures can be used if desired. Substantially equimolar amounts of reactants are preferably used although higher or lower amounts can be used if desired.

Application of Method A (Scheme 1) as described previously for the synthesis of compound of Formula I-A to the synthesis of compound of Formula XX is described below in Scheme 15. Compound of Formula XIX (compound of Formula II where m=1 and Z¹(X¹)ₘ—H equals fluoxetine hydrochloride) is reacted with compound of Formula XVIII (compound of Formula III where Y¹=Cl, G¹⁰, G¹¹, and G³⁰=O, t=1, and q=0 or 1) to afford compound of Formula XX (compound of Formula I-A where m=1, t=1, q=0 or 1, Z¹(X¹)ₘ=fluoxetine, and G¹⁰, G¹¹, and G³⁰⁼⁰):

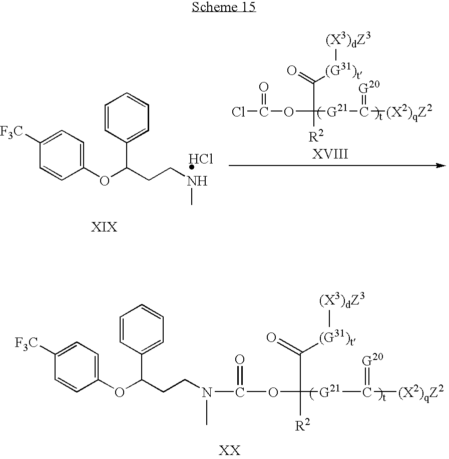

where Z²(X²)_{q}, Z³(X³)_{d}, d, t′, G²⁰, G²¹, G³¹, and R² are as defined previously for compound of Formula I-A.

In the reaction of a compound of Formula XVIII with a compound of Formula XIX to afford a compound of Formula XX, the following conditions can be used: suitable solvents for use in the above method include ethers such as tetrahydrofuran (THF), glyme, and the like; dimethylformamide (DMF); dimethylsulfoxide (DMSO); acetonitrile; chlorinated solvents such as methylene chloride (CH₂Cl₂) and chloroform (CHCl₃). If desired, mixtures of these solvents may be used, however, the preferred solvent is THF. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; 4-dimethylaminopyridine (DMAP) or pyridine. If desired, mixtures of these bases may be used, however, the preferred base is DMAP. The above method may be carried out at temperatures between about −78° C. and about 100° C. Preferably, the reaction is carried out at 22° C. Preparation of the compounds of the present invention by the above method is preferably carried out at about atmospheric pressure although higher or lower pressures can be used if desired. Substantially equimolar amounts of reactants are preferably used although higher or lower amounts can be used if desired. Generally, two equivalents of base are used per equivalent of starting material of compound of Formula XIX.

Application of Method A (Scheme 1) as described previously for the synthesis of compound of Formula I-A to the synthesis of compound of Formula XXII is described below in Scheme 16. Compound of Formula XXI (compound of Formula II where m=1 and Z¹(X¹)ₘ—H equals fluconazole) is reacted with compound of Formula XVIII (compound of Formula III where Y¹=Cl, G¹⁰, G¹¹, and G³⁰=O, t=1, and q=0 or 1) to afford compound of Formula XXII (compound of Formula I-A where m=1, t=1, q=0 or 1, Z¹(X¹)ₘ=fluconazole, and G¹⁰, G¹¹, and G³⁰=O):

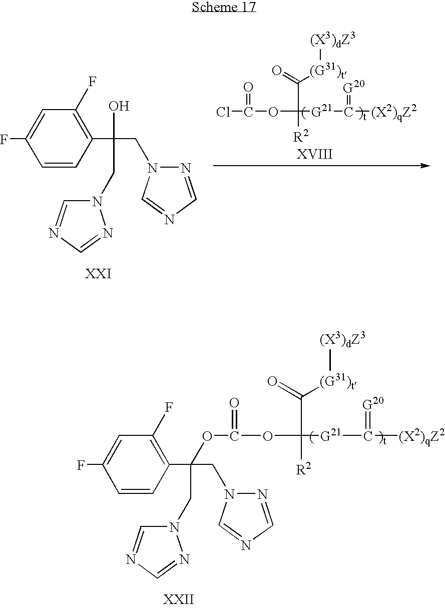

where Z²(X²)_{q}, Z³(X³)_{d}, d, t′, G²⁰, G²¹, G³¹, and R² are as defined previously for compound of Formula I-A.

In the reaction of a compound of Formula XXI with a compound of Formula XVIII to afford a compound of Formula XXII, the following conditions can be used: suitable solvents for use in the above method include ethers such as tetrahydrofuran (THF), glyme, and the like; dimethylformamide (DMF); dimethylsulfoxide (DMSO); acetonitrile; chlorinated solvents such as methylene chloride (CH₂Cl₂) and chloroform (CHCl₃). If desired, mixtures of these solvents may be used, however, the preferred solvent is THF. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; 4-dimethylaminopyridine (DMAP), potassium bis(trimethylsilyl)amide (KHMDS) or pyridine. If desired, mixtures of these bases may be used, however, the preferred base is KHMDS. The above method may be carried out at temperatures between about −78° C. and about 100° C. Preferably, the reaction is carried out at −78° C. to 0° C. Preparation of the compounds of the present invention by the above method is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula XXI.

Application of Method A (Scheme 1) as described previously for the synthesis of compound of Formula I-A to the synthesis of compound of Formula XXIV is described below in Scheme 17. Compound of Formula XXIII (compound of Formula II where m=1 and Z¹(X¹)ₘ—H equals nifedipine) is reacted with compound of Formula XVIII (compound of Formula III where Y¹=Cl, G¹⁰, G¹¹, and G³⁰=O, t=1, and q=0 or 1) to afford compound of Formula XXIV (compound of Formula I-A where m=1, t=1, q=0 or 1, Z¹(X¹)ₘ=nifedipine, and G¹⁰, G¹¹, and G³⁰=O):

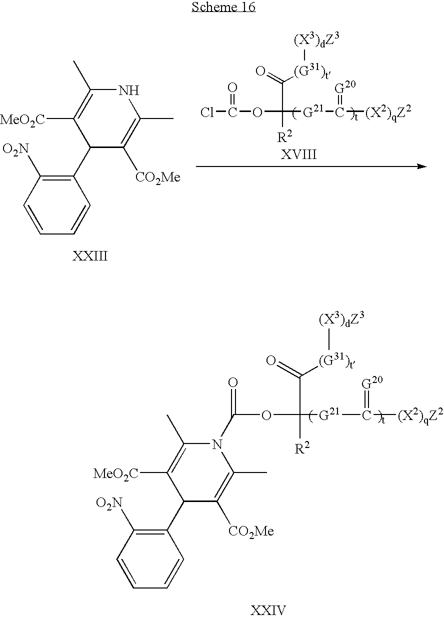

where Z²X²)_{q}, Z³X³)_{d}, d, t′, G²⁰, G²¹, G³¹, and R² are as defined previously for compound of Formula I-A.

In the reaction of a compound of Formula XXIII with a compound of Formula XVIII to afford a compound of Formula XXIV, the following conditions can be used: suitable solvents for use in the above method include ethers such as tetrahydrofuran (THF), glyme, and the like; dimethylformamide (DMF); dimethylsulfoxide (DMSO); acetonitrile; chlorinated solvents such as methylene chloride (CH₂Cl₂) and chloroform (CHCl₃). If desired, mixtures of these solvents may be used, however, the preferred solvent is THF. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine or diisopropylethylamine; an alkali metal carbonate such as sodium or potassium carbonate; 4-dimethylaminopyridine (DMAP), potassium bis(trimethylsilyl)amide (KHMDS) or pyridine. If desired, mixtures of these bases may be used, however, the preferred base is KHMDS. The above method may be carried out at temperatures between about −78° C. and about 100° C. Preferably, the reaction is carried out at −78° C. to 0° C. Preparation of the compounds of the present invention by the above method is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula XXIII.

Application of Method A (Scheme 1) as described previously for the synthesis of compound of Formula I-A to the synthesis of compound of Formula XXVI is described below in Scheme 18. Compound of Formula XXV (compound of Formula II where m=1 and Z¹(X¹)ₘ—H equals norfloxacin) is reacted with compound of Formula XVIII (compound of Formula III where Y′=Cl, G¹⁰, G¹¹, and G³⁰=O, t=1, and q=0 or 1) to afford compound of Formula XXVI (compound of Formula I-A where m=1, t

=1, q=0 or 1, Z¹(X¹)ₘ=norfloxacin, and G¹⁰, G¹¹, and G³⁰O):

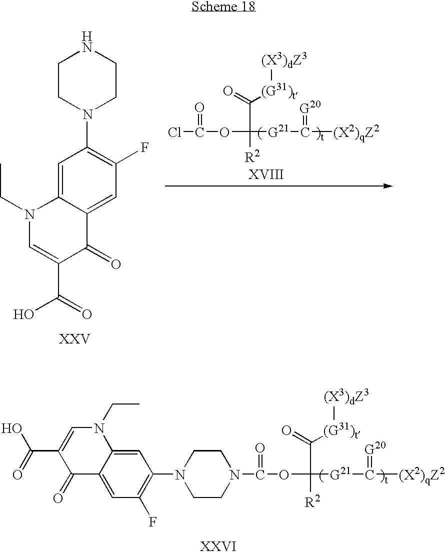

where Z²(X²)_{q}, Z³(X³)_{d}, d, t′, G²⁰, G²¹, G³′, and R² are as defined previously for compound of Formula I-A.

In the reaction of a compound of Formula XXV with a compound of Formula XVIII to afford a compound of Formula XXVI, the following conditions can be used: suitable solvents for use in the above method include ethers such as tetrahydrofuran (THF), glyme, and the like; dimethylformamide (DMF); dimethylsulfoxide (DMSO); acetonitrile; chlorinated solvents such as methylene chloride (CH₂Cl₂) and chloroform (CHCl₃) If desired, mixtures of these solvents may be used, however, the preferred solvent is CH₂Cl₂. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine, diisopropylethylamine, or trijsopropylamine; an alkali metal carbonate such as sodium or potassium carbonate; 4-dimethylaminopyridine (DMAP), potassium bis(trimethylsilyl)amide (KHMDS) or pyridine. If desired, mixtures of these bases may be used, however, the preferred base is triisopropylamine. The above method may be carried out at temperatures between about −78° C. and about 100° C. Preferably, the reaction is carried out at 22° C. Preparation of the compounds of the present invention by the above method is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula XXV.

Application of Method A (Scheme 1) as described previously for the synthesis of compound of Formula I-A to the synthesis of compound of Formula XXVIII is described below in Scheme 19. Compound of Formula XXVII (compound of Formula II where m=1 and Z¹(X¹)ₘ—H equals 4-acetamidophenol) is reacted with compound of Formula XVIII (compound of Formula III where Y′=Cl, G¹⁰, G¹¹, and G³⁰=O, t=1, and q=0 or 1) to afford compound of Formula XXVIII (compound of Formula I-A where m=1, t=1, q=0 or 1, Z¹(X¹)ₘ=4-acetamidophenol, and G¹⁰, G¹¹, and G³⁰=O):

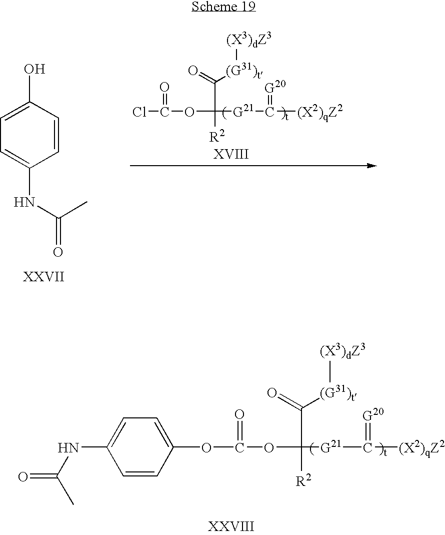

where Z²(X²)_{q}, Z³(X³)_{d}, d, t′, G²⁰, G²¹, G³¹, and R² are as defined previously for compound of Formula I-A.

In the reaction of a compound of Formula XXVII with a compound of Formula XVIII to afford a compound of Formula XXVIII, the following conditions can be used: suitable solvents for use in the above method include ethers such as tetrahydrofuran (THF), glyme, and the like; dimethylformamide (DMF); dimethylsulfoxide (DMSO); acetonitrile; chlorinated solvents such as methylene chloride (CH₂Cl₂) and chloroform (CHCl₃). If desired, mixtures of these solvents may be used, however, the preferred solvent is CH₂Cl₂. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine, diisopropylethylamine, or triisopropylamine; an alkali metal carbonate such as sodium or potassium carbonate; 4-dimethylaminopyridine (DMAP), potassium bis(trimethylsilyl)amide (KHMDS) or pyridine. If desired, mixtures of these bases may be used, however, the preferred base is potassium hydroxide. The above method may be carried out at temperatures between about −78° C. and about 100° C. Preferably, the reaction is carried out at 22° C. Preparation of the compounds of the present invention by the above method is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula XXVII.

Application of Method A (Scheme 1) as described previously for the synthesis of compound of Formula I-A to the synthesis of compound of Formula XXX is described below in Scheme 20. Compound of Formula XXIX (compound of Formula II where m=1 and Z¹(X¹)ₘ—H equals sulfamethoxazole) is reacted with compound of Formula XVIII (compound of Formula III where Y¹=Cl, G¹⁰, G¹¹, and G³⁰=O, t=1, and q=0 or 1) to afford compound of Formula XXX (compound of Formula I-A where m=1, t=1, q=0 or 1, Z¹(X¹)ₘ=sulfamethoxazole, and G¹⁰, G¹¹, and G³⁰=O):

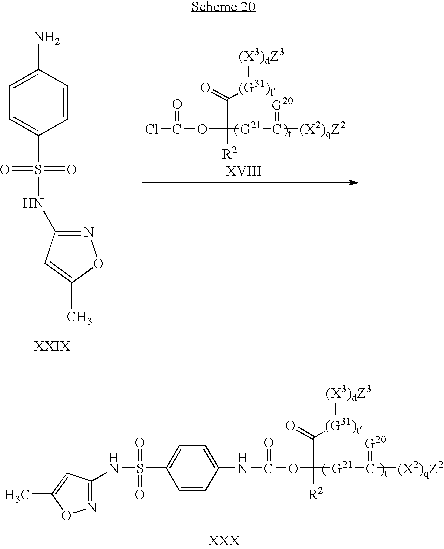

where Z²(X²)_{q}, Z³(X³)_{d}, d, t′, G²⁰, G²¹, G³¹, and R² are as defined previously for compound of Formula I-A.

In the reaction of a compound of Formula XXIX with a compound of Formula XVIII to afford a compound of Formula XXX, the following conditions can be used: suitable solvents for use in the above method include ethers such as tetrahydrofuran (THF), glyme, and the like; dimethylformamide (DMF); dimethylsulfoxide (DMSO); acetonitrile; chlorinated solvents such as methylene chloride (CH₂Cl₂) and chloroform (CHCl₃). If desired, mixtures of these solvents may be used, however, the preferred solvent is CH₂Cl₂. Suitable bases for use in the above process include, but are not limited to, metal hydrides such as sodium or potassium hydride; metal alkoxides such as sodium or potassium alkoxides; alkali metal hydroxides such as sodium or potassium hydroxide; tertiary amines such as triethylamine, diisopropylethylamine, or triisopropylamine; an alkali metal carbonate such as sodium or potassium carbonate; 4-dimethylaminopyridine (DMAP), potassium bis(trimethylsilyl)amide (KHMDS) or pyridine. If desired, mixtures of these bases may be used, however, the preferred base is triisopropylamine. The above method may be carried out at temperatures between about −78° C. and about 100° C. Preferably, the reaction is carried out at 22° C. Preparation of the compounds of the present invention by the above method is preferably carried out at about atmospheric pressure although higher or lower pressures can be used if desired. Substantially equimolar amounts of reactants are preferably used although higher or lower amounts can be used if desired. Generally, one equivalent of base is used per equivalent of starting material of compound of Formula XXIX.

Following the general methods described hereinbefore, the following compounds of Formula VII (where R²=H) as listed in Table 1 were prepared.

TABLE 1

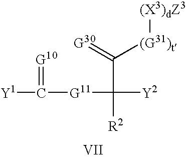

Listing of Compounds of Formula VII

| Cmpd # | Y¹ | G¹⁰ | G¹¹ | G³⁰ | Y² | G³¹ | t′ | d | (X³)_{d}Z³ |
|---|---|---|---|---|---|---|---|---|---|
| 1-1 | Cl | O | O | O | Cl | O | 1 | 0 | benzyl |
| 1-2 | Cl | O | O | O | Cl | O | 1 | 0 | ethyl |
| 1-3 | Cl | O | O | O | Cl | O | 1 | 0 | methyl |
| 1-4 | Cl | O | O | O | Cl | O | 1 | 0 | isopropyl |
| 1-5 | Cl | O | O | O | Cl | O | 1 | 0 | tert-butyl |
| 1-6 | Cl | O | O | O | Cl | O | 1 | 0 | n-butyl |

The following Examples are provided for guidance to the practitioner in order to practice the invention.

### EXAMPLE 1

Benzyl 2-chloro-2-[(chlorocarbonyl)oxy]acetate (Compound 1-1 of Table 1)

To a 3-neck round-bottom flask, equipped with nitrogen inlet, a thermometer and a solid addition funnel, was added benzyl glyoxylate (50.9 g, 300 mmol), pyridine (2.5 mL, 31.0 mmol) and 1500 mL of carbon tetrachloride. The solution was cooled with dry ice/acetone to −20° C. and triphosgene (230 g, 770 mmol) was added over 5 minutes, maintaining the temperature between −10° C. and −20° C. The reaction was gradually warmed to room temperature over 2 h, then warmed to 50° C. and was stirred at that temperature for 1 h. The reaction was then cooled and placed in the freezer overnight. The precipitates were filtered by gravity, washing with carbon tetrachloride. The solvent was removed in vacuo, with low heat, to yield 58 g of the desired benzyl 2-chloro-2-[(chlorocarbonyl)oxy]acetate as a clear colorless oil. ¹H-NMR (300 MHz, CDCl₃) δ (ppm): 5.24 (s, 2H), 6.45 (s, 1H), 7.32 (s, 5H).

### EXAMPLE 2

Ethyl 2-chloro-2-[(chlorocarbonyl)oxy]acetate (Compound 1-2 of Table 1)

Pyridine (0.145 mL, 1.79 mmol) was added to a solution of polymeric ethyl glyoxylate (18.51 g, 181 mmol) and triphosgene (48.5 g, 163 mmol) in dry THF at room temperature in a flask fitted with a reflux condenser and connected to a N₂ bubbler. After 10 min the flask was placed in a pre-heated 65° C. oil bath. After 21 h, the reaction was allowed to cool to room temperature, and then the mixture was concentrated under vacuum. Ether was added to the residue, the mixture was filtered through Celite, and the filtrate was concentrated, affording 34.6 g (87% yield) of a yellow oil. ¹H-NMR (300 MHz, CDCl₃) δ (ppm): 1.36 (t, 3H) 4.36 (q, 2H), 6.48 (s, 1H).

### EXAMPLE 3

Methyl 2-chloro-2-[(chlorocarbonyl)oxy]acetate (Compound 1-3 of Table 1)

The title compound was prepared according to the procedure described in Example 2 above, except methyl glyoxylate was substituted for ethyl glyoxylate. 1H-NMR (300 MHz, CDCl₃) δ (ppm): 3.92 (s, 3H), 6.52 (s, 1H).

### EXAMPLE 4

Isopropyl 2-chloro-2-[(chlorocarbonyl)oxy]acetate (Compound 1-4 of Table 1)

The title compound was prepared according to the procedure described in Example 2 above, except isopropyl glyoxylate was substituted for ethyl glyoxylate. ¹H-NMR (300 MHz, CDCl₃) δ (ppm): 1.33 (s, 3H), 1.35 (s, 3H), 5.2 (q, 1H), 6.44 (s, 1H).

### EXAMPLE 5

tert-Butyl 2-chloro-2-[(chlorocarbonyl)oxy]acetate (Compound 1-5 of Table 1)

The title compound was prepared according to the procedure described in Example 2 above, except tert-butyl glyoxylate was substituted for ethyl glyoxylate. ¹H-NMR (300 MHz, CDCl₃) δ (ppm): 1.53 (s, 9H), 6.35 (s, 1H).

### EXAMPLE 6

n-Butyl 2-chloro-2-[(chlorocarbonyl)oxy]acetate (Compound 1-6 of Table 1)

The title compound was prepared according to the procedure described in Example 2 above, except n-butyl glyoxylate was substituted for ethyl glyoxylate. ¹H-NMR (300 MHz, CDCl₃) δ (ppm): 0.94 (t, 3H), 1.40 (q, 2H), 1.68 (m, 2H), 4.28 (m, 2H), 6.49 (s, 1H).

Following the general methods described hereinbefore, the following compounds of Formula V (where R²=H) as listed in Table 2 were prepared.

TABLE 2

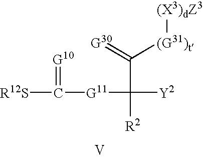

Listing of Compounds of Formula V

| Cmpd # | R¹² | G¹⁰ | G¹¹ | G³⁰ | Y² | G³¹ | t′ | d | (X³)_{d}Z³ |
|---|---|---|---|---|---|---|---|---|---|
| 2-1 | Et | O | O | O | Cl | O | 1 | 0 | ethyl |
| 2-2 | Et | O | O | O | I | O | 1 | 0 | ethyl |
| 2-3 | Et | O | O | O | Cl | O | 1 | 0 | n-butyl |
| 2-4 | Et | O | O | O | I | O | 1 | 0 | n-butyl |
| 2-5 | Et | O | O | O | Cl | O | 1 | 0 | isopropyl |
| 2-6 | Et | O | O | O | I | O | 1 | 0 | isopropyl |

The following Examples are provided for guidance to the practitioner in order to practice the invention.

### EXAMPLE 7

Chloro-ethylsulfanylcarbonyloxy-acetic acid ethyl ester (Compound 2-1 of Table 2)

A 1000 mL round bottom flask was charged with sodium ethylthiolate (13.3 g, 158 mmol) and 500 mL of dry diethyl ether. The mixture was cooled to −70° C. in an acetone dry ice bath. Ethyl 2-chloro-2-[(chlorocarbonyl)oxy]acetate (32.8 g, 155 mmol) was added as a solution in 20 mL of diethyl ether over 1.5 h at such a rate that the reaction temperature did not exceed −65° C. The reaction was allowed to warm to room temperature and stir for 16 h. The reaction was vacuum filtered, the filtrate dried (MgSO₄), gravity filtered, and-concentrated under reduced pressure to yield 33.5 g of a clear liquid. ¹H-NMR (300 MHz, CDCl₃) δ (Ppm): 1.33 (m, 6H), 2.94 (q, 2H), 4.31 (q, 2H), 6.65 (s, 1H).

### EXAMPLE 8

Iodo-ethylsulfanylcarbonyloxy-acetic acid ethyl ester (Compound 2-2 of Table 2)

To a stirred solution of chloro-ethylsulfanylcarbonyloxy-acetic acid ethyl ester (33.5 g, 148 mmol) in 160 mL of dry acetone was added NaI (28.8. g, 192 mmol). The mixture was stirred at room temperature for 4 h. The acetone was removed and the remaining slurry was diluted with 100 mL of diethyl ether. The mixture was filtered through Celite and concentrated under reduced pressure to yield a brown liquid. The liquid was redissolved in 50 mL of diethyl ether and gravity filtered to afford 37.1 g of a brown liquid. ¹H-NMR (300 MHz, CDCl₃) δ (ppm): 1.33 (m, 6H), 2.95 (q, 2H), 4.30 (q, 2H), 7.21 (s, 1H).

### EXAMPLE 9

Chloro-ethylsulfanylcarbonyloxy-acetic acid butyl ester (Compound 2-3 of Table 2)

The title compound was prepared according to the procedure described in Example 7 above except for the substitution of n-butyl 2-chloro-2-[(chlorocarbonyl)oxy]acetate for ethyl 2-chloro-2-[(chlorocarbonyl)oxy]acetate. ¹H-NMR (300 MHz, CDCl₃) δ (ppm): 0.95 (t, 3H), 1.33-1.43 (m, 5H), 1.67-1.72 (m, 2H), 2.93 (q, 2H), 4.25-4.30 (m, 2H), 6.65 (s, 1H).

### EXAMPLE 10

Iodo-ethylsulfanylcarbonyloxy-acetic acid butyl ester (Compound 2-4 of Table 2)

The title compound was prepared according to the procedure described in Example 8 above except for the substitution of chloro-ethylsulfanylcarbonyloxy-acetic acid butyl ester for chloro-ethylsulfanylcarbonyloxy-acetic acid ethyl ester. ¹H-NMR (300 MHz, CDCl₃) δ (ppm): 0.95 (t, 3H), 1.32-1.43 (m, 5H), 1.65-1.70 (m, 2H), 2.92-2.95 (m, 2H), 4.22-4.26 (m, 2H), 7.21 (s, 1H).

### EXAMPLE 11

Chloro-ethylsulfanylcarbonyloxy-acetic acid isopropyl ester (Compound 2-5 of Table 2)

The title compound was prepared according to the procedure described in Example 7 above except for the substitution of isopropyl 2-chloro-2-[(chlorocarbonyl)oxy]acetate for ethyl 2-chloro-2-[(chlorocarbonyl)oxy]acetate. ¹H-NMR (300 MHz, CDCl₃) δ (ppm): 1.30 (t, 9H), 2.90 (q, 2H), 5.10 (s, 1H), 6.60 (s, 1H).

### EXAMPLE 12

Iodo-ethylsulfanylcarbonyloxy-acetic acid isopropyl ester (Compound 2-6 of Table 2)

The title compound was prepared according to the procedure described in Example 8 above except for the substitution of chloro-ethylsulfanylcarbonyloxy-acetic acid isopropyl ester for chloro-ethylsulfanylcarbonyloxy-acetic acid ethyl ester. ¹H-NMR (300 MHz, CDCl₃) 8 (ppm): 1.30 (t, 9H), 2.90 (q, 2H), 5.10 (s, 1H), 7.15 (s, 1H).

Following the general methods described hereinbefore, the following compounds of Formula IV (where G¹⁰, G¹¹, G²⁰, G²¹, G³⁰, and G³¹=O, and R²=H) as listed in Table 3 were prepared.

TABLE 3

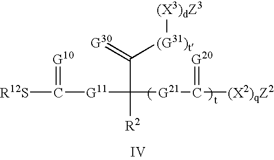

Listing of Compounds of Formula IV

| Cmpd # | R¹² | t′ | d | (X³)_{d}Z³ | t | q | (X²)_{q}Z² |
|---|---|---|---|---|---|---|---|
| 3-1 | Et | 1 | 0 | ethyl | 1 | 0 | 2-propyl |
| 3-2 | Et | 1 | 0 | ethyl | 1 | 0 | t-butyl |
| 3-3 | Et | 1 | 0 | ethyl | 1 | 0 | ethyl |
| 3-4 | Et | 1 | 0 | ethyl | 1 | 0 | 2-ethoxyphenyl |
| 3-5 | Et | 1 | 0 | ethyl | 1 | 0 | phenyl |
| 3-6 | Et | 1 | 0 | ethyl | 1 | 0 | 2,4-dichlorophenoxymethyl |
| 3-7 | Et | 1 | 0 | ethyl | 1 | 0 | 3-(2,4-dichlorophenoxy)propyl |
| 3-8 | Et | 1 | 0 | ethyl | 1 | 0 | 1-(2,4-dichlorophenoxy)ethyl |
| 3-9 | Et | 1 | 0 | ethyl | 1 | 0 | 2,5-dichloro-6-methoxyphenyl |
| 3-10 | Et | 1 | 0 | ethyl | 1 | 0 | 2,4,6-trimethylphenyl |
| 3-11 | Et | 1 | 0 | 2-propyl | 1 | 0 | phenyl |
| 3-12 | Et | 1 | 0 | 2-propyl | 1 | 0 | t-butyl |
| 3-13 | Et | 1 | 0 | 2-propyl | 1 | 0 | 1-methyl-1-cyclopropyl |
| 3-14 | Et | 1 | 0 | 2-propyl | 1 | 0 | 2-propyl |
| 3-15 | Et | 1 | 0 | 2-propyl | 1 | 0 | ethyl |
| 3-16 | Et | 1 | 0 | 2-propyl | 1 | 0 | N-acetyl-N-methyl-aminomethyl |
| 3-17 | Et | 1 | 0 | n-butyl | 1 | 0 | (diethoxyphosphoryl)methyl |
| 3-18 | Et | 1 | 0 | n-butyl | 1 | 0 | t-butyl |
| 3-19 | Et | 1 | 0 | 2-propyl | 1 | 0 | 3,7-dichloro-8-quinoline |

The following Examples are provided for guidance to the practitioner in order to practice the invention.

### EXAMPLE 13

2-Methylpropanoic acid ethoxycarbonyl-ethylsulfanylcarbonyloxy-methyl ester (Compound 3-1 of Table 3)

To a stirred ice cold solution of iodo-ethylsulfanylcarbonyloxy-acetic acid ethyl ester (5.4 g, 17.0 mmol) in 20 mL of dry THF was added 2-methylpropanoic acid (1.94 g, 22.1 mmol) followed by DIEA (2.85 g, 22.1 mmol). The reaction was allowed to stir at room temperature for 16 h. The reaction was diluted with 100 mL of diethyl ether, gravity filtered, and concentrated under reduced pressure. The liquid was suction filtered through a pad of flash grade silica gel and eluted with 20% methylene chloride/hexanes. ¹H-NMR (300 MHz, CDCl₃) δ (ppm): 1.24 (m, 6H), 1.33 (m, 6H), 2.66 (m, 1H), 2.90 (q, 2H), 4.28 (q, 2H), 5.92 (s, 1H).

### EXAMPLE 14

Pivalic acid ethoxycarbonyl-ethylsulfanylcarbonyloxy-methyl ester (Compound 3-2 of Table 3)

The title compound was prepared according to the procedure described in Example 13 above except for the substitution of pivalic acid for 2-methylpropanoic acid. ¹H-NMR (300 MHz, CDCl₃) 5 (ppm): 1.25 (s, 9H), 1.33 (m, 6H), 2.88 (q, 2H), 4.28 (q, 2H), 6.82 (s, 1H).

### EXAMPLE 15

Propionic acid ethoxycarbonyl-ethylsulfanylcarbonyloxy-methyl ester (Compound 3-3 of Table 3)

The title compound was prepared according to the procedure described in Example 13 above except for the substitution of propionic acid for 2-methylpropanoic acid. ¹H-NMR (300 MHz, CDCl₃) δ (ppm): 1.16 (t, 3H), 1.30 (m, 6H), 2.47 (q, 2H), 2.93 (q, 2H), 4.29 (q, 2H), 6.94 (s, 1H).

### EXAMPLE 16

2-Ethoxybenzoic acid ethoxycarbonyl-ethylsulfanylcarbonyloxy-methyl ester (Compound 3-4 of Table 3)

The title compound was prepared according to the procedure described in Example 13 above except for the substitution of 2-ethoxybenzoic acid for 2-methylpropanoic acid. ¹H-NMR (300 MHz, CDCl₃) δ (ppm): 1.23 (m, 6H), 1.44 (t, 3H), 2.92 (q, 2H), 4.12 (q, 2H), 4.31 (q, 2H), 6.96 (m, 2H), 7.15 (s, 1H), 7.43 (t, 1H), 7.92 (d, 1H).

### EXAMPLE 17

Benzoic acid ethoxycarbonyl-ethylsulfanylcarbonyloxy-methyl ester (Compound 3-5 of Table 3)

The title compound was prepared according to the procedure described in Example 13 above except for the substitution of benzoic acid for 2-methylpropanoic acid. ¹H-NMR (300 MHz, CDCl₃) δ (ppm): 1.32 (m, 6H), 2.93 (q, 2H), 4.35 (q, 2H), 7.19 (s, 1H), 7.35 (t, 2H), 7.48 (t, 1H), 8.10 (d, 2H).

### EXAMPLE 18

2,4-Dichlorophenoxyacetic acid ethoxycarbonyl-ethylsulfanylcarbonyloxy-methyl ester (Compound 3-6 of Table 3)

The title compound was prepared according to the procedure described in Example 13 above except for the substitution of 2,4-dichlorophenoxyacetic acid for 2-methylpropanoic acid. ¹H-NMR (300 MHz, CDCl₃) δ (ppm): 1.32 (m, 6H), 2.92 (q, 2H), 4.29 (q, 2H), 4.82 (s, 2H), 6.83 (d, 1H), 6.95 (s, 1H), 7.24 (dd, 1H), 7.38 (s, 1H).

### EXAMPLE 19

4-(2,4-Dichlorophenoxy)butyric acid ethoxycarbonyl-ethylsulfanylcarbonyloxy-methyl ester (Compound 3-7 of Table 3)

The title compound was prepared according to the procedure described in Example 13 above except for the substitution of 4-(2,4-diclorophenoxy)butyric acid for 2-methylpropanoic acid. ¹H-NMR (300 MHz, CDCl₃) 8 (ppm): 1.32 (m, 6H), 2.18 (m, 2H), 2.76 (t, 2H), 2.97 (q, 2H), 4.02 (q, 2H), 4.31 (q, 2H), 6.83 (d, 1H), 6.86 (s, 1H), 7.15 (d, 1H), 7.35 (d, 1H).

### EXAMPLE 20

2′-(2,4-Dichlorophenoxy)propionic acid ethoxycarbonyl-ethylsulfanylcarbonyloxy-methyl ester (Compound 3-8 of Table 3)

The title compound was prepared according to the procedure described in Example 13 above except for the substitution of 2′-(2,4-diclorophenoxy)propionic acid for 2-methylpropanoic acid. ¹H-NMR (300 MHz, CDCl₃) 5 (ppm): 1.32 (m, 6H), 1.74 (m, 3H), 2.91 (q, 2H), 4.26 (q, 2H), 4.88 (m, 1H), 6.88 (m, 1H), 6.95 (s, 1H), 7.18 (d, 1H), 7.38 (s, 1H).

### EXAMPLE 21

2,5-Dichloro-6-methoxybenzoic acid ethoxycarbonyl-ethylsulfanylcarbonyloxy-methyl ester (Compound 3-9 of Table 3)

The title compound was prepared according to the procedure described in Example 13 above except for the substitution of 2,5-dichloro-6-methoxybenzoic acid for 2-methylpropanoic acid. ¹H-NMR (300 MHz, CDCl₃) δ (ppm): 1.34 (m, 6H), 2.93(q, 2H), 3.94 (s, 3H), 4.34 (q, 2H), 7.16 (d, 1H), 7.18 (s, 1H), 7.39 (d, 1H).

### EXAMPLE 22

2,4,6-Trimethylbenzoic acid ethoxycarbonyl-ethylsulfanylcarbonyloxy-methyl ester (Compound 3-10 of Table 3)

The title compound was prepared according to the procedure described in Example 13 above except for the substitution of 2,4,6-trimethylbenzoic acid for 2-methylpropanoic acid. ¹H-NMR (300 MHz, CDCl₃) δ (ppm): 1.33 (m, 6H), 2.27 (s, 3H), 2.34 (s, 6H), 2.90 (q, 2H), 4.31 (q, 2H), 6.86 (s, 2H), 7.16 (s, 1H).

### EXAMPLE 23

Benzoic acid isopropoxycarbonyl-ethylsulfanylcarbonyloxy-methyl ester (Compound 3-11 of Table 3)

To a stirred ice cold solution of iodo-ethylsulfanylcarbonyloxy-acetic acid isopropyl ester (3.1 g, 9.3 mmol) in 40 mL of dry THF was added benzoic acid (1.5 g, 12.0 mmol) followed by DIEA (2.1 mL, 12.0 mmol). The reaction was allowed to stir at room temperature for 16 h. The solvent was removed under reduced pressure. The residue was dissolved in ether and washed 3 times with a saturated solution of sodium bicarbonate and once with brine. The ether layer was dried over MgSO₄ and concentrated to yield 2.0 g (66%) of the desired product, which was used without further purification. 1H-NMR (300 MHz, CDCl₃) δ (ppm): 1.30 (m, 9H), 2.90 (q, 2H), 5.15 (m, 1H), 7.13 (s, 1H), 7.55 (m, 3H), 8.10 (m, 2H).

### EXAMPLE 24

Pivalic acid isopropoxycarbonyl-ethyl sulfanylcarbonyloxy-methyl ester (Compound 3-12 of Table 3)

The title compound was prepared according to the procedure described in Example 23 above except for the substitution of pivalic acid for benzoic acid. ¹H-NMR (300 MHz, CDCl₃) 5 (ppm): 1.30 (m, 18H), 2.90 (q, 2H), 5.10 (m, 1H), 6.85 (s, 1H).

### EXAMPLE 25

1-Methyl-1-cyclopropanecarboxylic acid isopropoxycarbonyl-ethylsulfanylcarbonyloxy-methyl ester (Compound 3-13 of Table 3)

The title compound was prepared according to the procedure described in Example 23 above except for the substitution of 1-methyl-1-cyclopropanecarboxylic acid for benzoic acid. ¹H-NMR (300 MHz, CDCl₃) δ (ppm): 0.75 (d, 2H), 1.35 (m, 14H), 2.90 (m, 2H), 5.10 (m, 1H), 6.85 (s, 1H).

### EXAMPLE 26

Isobutyric acid isopropoxycarbonyl-ethylsulfanylcarbonyloxy-methyl ester (Compound 3-14 of Table 3)

The title compound was prepared according to the procedure described in Example 23 above except for the substitution of isobutyric acid for benzoic acid. ¹H-NMR (300 MHz, CDCl₃) δ (ppm): 1.25 (m, 15H), 2.65 (m, 1H), 2.90 (m, 2H), 5.10 (m, 1H), 6.90 (s, 1H).

### EXAMPLE 27

Propionic acid isopropoxycarbonyl-ethylsulfanylcarbonyloxy-methyl ester (Compound 3-15 of Table 3)

The title compound was prepared according to the procedure described in Example 23 above except for the substitution of propionic acid for benzoic acid. ¹H-NMR (300 MHz, CDCl₃) δ (ppm): 1.15 (t, 3H), 1.30 (m, 9H), 2.45 (q, 2H), 2.90 (q, 2H), 5.10 (m, 1H), 6.90 (s, 1H).

### EXAMPLE 28

Acetyl methyl carbamic acid isopropoxycarbonyl-ethylsulfanylcarbonyloxy-methyl ester (Compound 3-16 of Table 3)

The title compound was prepared according to the procedure described in Example 23 above except for the substitution of acetyl methyl carbamic acid for benzoic acid. ¹H-NMR (300 MHz; CDCl₃) & (ppm): 1.30 (m, 9H), 2.10 (d, 3H), 2.90 (m, 2H), 3.10 (d, 3H), 4.20 (m, 2H), 5.10 (m, 1H), 6.90 (s, 1H).

### EXAMPLE 29

Diethylphosphonoacetic acid butoxycarbonyl-ethylsulfanylcarbonyloxy-methyl ester (Compound 3-17 of Table 3)

The title compound was prepared according to the procedure described in Example 13 above except for the substitution of diethylphosphonoacetic acid for 2-methylpropanoic acid and iodoethylsulfanylcarbonyloxy-acetic acid butyl ester for iodoethylsulfanylcarbonyloxy-acetic acid ethyl ester. ¹H-NMR (300 MHz, CD₃OD) δ (ppm): 1.35 (t, 6H), 2.89 (q, 2H), 3.03 (d, 2H), 4.18 (q, 4H), 5.84 (s, 2H).

### EXAMPLE 30

Pivalic acid butoxycarbonyl-ethylsulfanylcarbonyloxy-methyl ester (Compound 3-18 of Table 3)

The title compound was prepared according to the procedure described in Example 13 above except for the substitution of pivalic acid for 2-methylpropanoic acid and iodoethylsulfanylcarbonyloxy-acetic acid butyl ester for iodoethylsulfanylcarbonyloxy-acetic acid ethyl ester. ¹H-NMR (300 MHz, CD₃OD) δ (ppm): 1.25 (s, 9H), 1.31-1.45 (m, 8H), 1.65 (m 2H), 2.95 (q, 2H), 4.25 (m, 2H), 6.92 (s, 1H).

### EXAMPLE 31

3,7-Dichloro-8-quinolinecarboxylic acid isopropoxycarbonyl-ethylsulfanylcarbonyloxy-methyl ester (Compound 3-19 of Table 3)

The title compound was prepared according to the procedure described in Example 23 above except for the substitution of 3,7-dichloro-8-quinolinecarboxylic acid for benzoic acid. ¹H-NMR (300 MHz, CDCl₃) d (ppm): 1.35 (m, 9H), 2.95 (q, 2H), 5.15 (m, 1H), 7.25 (s, 1H), 7.57 (d, 1H), 7.76 (d, 1H), 8.14 (s, 1H) 8.83 (s, 1H).

Following the general methods described hereinbefore, the following compounds of Formula III (where G¹⁰, G¹¹, G²⁰, G²¹, G³⁰, and G³¹=O and R²=H) as listed in Table 4 were prepared.

TABLE 4

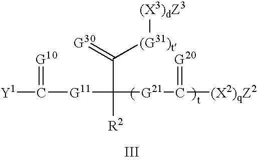

Listing of Compounds of Formula III

| Cmpd # | Y¹ | t′ | d | (X³)_{d}Z³ | t | q | (X²)_{q}Z² |
|---|---|---|---|---|---|---|---|
| 4-1 | Cl | 1 | 0 | ethyl | 1 | 0 | 2-propyl |
| 4-2 | Cl | 1 | 0 | ethyl | 1 | 0 | t-butyl |
| 4-3 | Cl | 1 | 0 | ethyl | 1 | 0 | ethyl |
| 4-4 | Cl | 1 | 0 | ethyl | 1 | 0 | 2-ethoxyphenyl |
| 4-5 | Cl | 1 | 0 | ethyl | 1 | 0 | phenyl |
| 4-6 | Cl | 1 | 0 | ethyl | 1 | 0 | 3-(2,4-dichlorophenoxy)propyl |
| 4-7 | Cl | 1 | 0 | ethyl | 1 | 0 | 1-(2,4-dichlorophenoxy)ethyl |
| 4-8 | Cl | 1 | 0 | ethyl | 1 | 0 | 2,5-dichloro-6-methoxyphenyl |
| 4-9 | Cl | 1 | 0 | ethyl | 1 | 0 | 2,4,6-trimethylphenyl |
| 4-10 | Cl | 1 | 0 | n-butyl | 1 | 0 | diethylphosphonomethyl |
| 4-11 | Cl | 1 | 0 | n-butyl | 1 | 0 | t-butyl |
| 4-12 | Cl | 1 | 0 | 2-propyl | 1 | 0 | 2-propyl |
| 4-13 | Cl | 1 | 0 | n-butyl | 1 | 0 | 2-propyl |
| 4-14 | Cl | 1 | 0 | 2-propyl | 1 | 0 | phenyl |
| 4-15 | Cl | 1 | 0 | 2-propyl | 1 | 0 | undecanyl |
| 4-16 | Cl | 1 | 0 | 2-propyl | 1 | 1 | 1-[4-(2-methylpropyl)phenyl]ethyl |

The following Examples are provided for guidance to the practitioner in order to practice the invention.

### EXAMPLE 32

2-Methylpropionic acid ethoxycarbonyl-chlorocarbonyloxy-methyl ester (Compound 4-1 of Table 4)

A 100 mL round bottom flask was charged with 2-methylpropionic acid ethoxycarbonyl-ethyl sulfanylcarbonyloxy-methyl ester (4.4 g, 15.8 mmol) and cooled to 5° C. Sulfuryl chloride (2.70 g, 20.0 mmol) was added over 1 min. After 30 min of stirring, the cooling bath was removed and the reaction was allowed to stir for 3 h at room temperature and then placed under vacuum. The material was used without purification. ¹H-NMR (300 MHz, CDCl₃) δ (ppm): 1.26 (m, 9H), 2.58 (m, 1H), 4.32 (q, 2H), 6.83 (s, 1H).

### EXAMPLE 33

Pivalic acid ethoxycarbonyl-chlorocarbonyloxy methyl ester (Compound 4-2 of Table 4)

The title compound was prepared according to the procedure described in Example 32 above except for the substitution of pivalic acid ethoxycarbonyl-ethylsulfanylcarbonyloxy methyl ester for isobutyric acid ethoxycarbonyl-ethylsulfanylcarbonyloxy-methyl ester. ¹H-NMR (300 MHz, CDCl₃) δ (ppm): 1.25 (s, 9H), 1.33 (t, 3H), 4.32 (q, 2H), 6.78 (s, 1H).

### EXAMPLE 34

Propionic acid ethoxycarbonyl-chlorocarbonyloxy-methyl ester (Compound 4-3 of Table 4)

The title compound was prepared according to the procedure described in Example 32 above except for the substitution propionic acid ethoxycarbonyl-ethylsulfanylcarbonyloxy-methyl ester for isobutyric acid ethoxycarbonyl-ethylsulfanylcarbonyloxy-methyl ester, using 1.4 equiv of sulfuryl chloride and starting the reaction at 5° C. ¹H-NMR (300 MHz, CDCl₃) δ (ppm): 1.21 (t, 3H), 1.35 (t, 3H), 2.53 (q, 2H), 4.32 (q, 2H), 6.84 (s, 1H).

### EXAMPLE 35

2-Ethoxybenzoic acid ethoxycarbonyl-chlorocarbonyloxy-methyl ester (Compound 4-4 of Table 4)

The title compound was prepared according to the procedure described in Example 32 above except for the substitution 2-ethoxybenzoic acid ethoxycarbonylethylsulfanyl-carbonyloxy methyl ester for isobutyric acid ethoxycarbonyl-ethylsulfanylcarbonyloxy-methyl ester, using 1.4 equiv of sulfuryl chloride and starting the reaction at 5° C. ¹H-NMR (300 MHz, CDCl₃) δ (ppm): 1.36 (t, 3H), 1.45 (t, 3H), 4.13 (q, 2H), 4.36 (q, 2H), 6.95 (m, 2H), 7.02 (s, 1H), 7.47 (t, 1H), 7.91(d, 1H).

### EXAMPLE 36

Benzoic acid ethoxycarbonyl-chlorocarbonyloxy-methyl ester (Compound 4-5 of Table 4)

The title compound was prepared according to the procedure described in Example 32 above except for the substitution benzoic acid ethoxycarbonyl-ethylsulfanylcarbonyloxy-methyl ester for isobutyric acid ethoxycarbonyl-ethylsulfanylcarbonyloxy-methyl ester, using 1.2 equiv of sulfuryl chloride and starting the reaction at 5° C. ¹H-NMR (300 MHz, CDCl₃) δ (ppm): 1.36 (t, 3H), 4.32 (q, 2H), 7.09 (s, 1H), 7.50 (t, 2H), 7.63 (t, 1H), 8.10 (d, 2H).

### EXAMPLE 37

2,4-Dichlorophenoxybutyric acid ethoxycarbonyl-chlorocarbonyloxy-methyl ester (Compound 4-6 of Table 4)

The title compound was prepared according to the procedure described in Example 32 above except for the substitution 2,4-dichlorophenoxybutyric acid ethoxycarbonylethyl-sulfanylcarbonyloxy-methyl ester for isobutyric acid ethoxycarbonyl ethylsulfanylcarbonyl-oxy-methyl ester, using 1.45 equiv of sulfuryl chloride and starting the reaction at 5° C. ¹H-NMR (300 MHz, CDCB) δ (ppm): 1.34 (t, 3H), 2.21 (m, 2H), 2.56 (t, 2H), 4.07 (t, 2H), 4.31 (q, 2H), 6.83 (d, 1H), 6.86 (s, 1H), 7.17 (d, 1H), 7.35 (d, 1H).

### EXAMPLE 38

2′-(2,4-Dichlorophenoxy)propionic acid ethoxycarbonyl-chlorocarbonyloxy-methyl ester (Compound 4-7 of Table 4)

The title compound was prepared according to the procedure described in Example 32 above except for the substitution 2′-(2,4-dichlorophenoxy)propionic acid ethoxycarbonyl-ethylsulfanylcarbonyloxy-methyl ester for isobutyric acid ethoxycarbonyl-ethylsulfanylcarbonyloxy-methyl ester, using 1.80 equiv of sulfuryl chloride and starting the reaction at 5° C. ¹H-NMR (300 MHz, CDCl₃) δ (ppm): 1.37 (t, 3H), 1.92 (m, 3H), 4.33 (q, 2H), 4.83 (m, 1H), 6.84 (m, 2H), 7.16 (dd, if), 7.38 (d, 1H).

### EXAMPLE 39

2,5-Dichloro-6-methoxybenzoic acid ethoxycarbonyl-chlorocarbonyloxy-methyl ester (Compound 4-8 of Table 4)

The title compound was prepared according to the procedure described in Example 32 above except for the substitution 2,5-dichloro-6-methoxybenzoic acid ethoxycarbonyl-ethylsulfanylcarbonyloxy-methyl ester for isobutyric acid ethoxycarbonyl-ethylsulfanylcarbonyloxy-methyl ester, using 1.45 equiv of sulfuryl chloride and starting the reaction at 5° C. ¹H-NMR (300 MHz, CDCB) δ (ppm): 1.34 (t, 3H), 3.95 (s, 3H), 4.36 (q, 2H), 7.08 (s, 1H), 7.18 (d, 1H), 7.42 (d, 1H).

### EXAMPLE 40

2,4,6-Trimethylbenzoic acid ethoxycarbonyl-chlorocarbonyloxy-methyl ester (Compound 4-9 of Table 4)

The title compound was prepared according to the procedure described in Example 32 above except for the substitution of 2,4,6-trimethylbenzoic acid ethoxycarbonyl-ethylsulfanylcarbonyloxy-methyl ester for isobutyric acid ethoxycarbonyl-ethylsulfanylcarbonyloxy-methyl ester, using 1.45 equiv of sulfuryl chloride and starting the reaction at 5° C. ¹H-NMR (300 MHz, CDCl₃) 8 (Ppm): 1.34 (t, 3H), 2.28 (s, 3H), 2.35 (s, 6H), 4.34 (q, 2H), 6.89 (s, 1H), 7.06 (s, 1H), 7.12 (s, 1H).

### EXAMPLE 41

Diethylphosphonoacetic acid butoxycarbonyl-chlorocarbonyloxy-methyl ester (Compound 4-10 of Table 4)

The title compound was prepared according to the procedure described in Example 32 above except for the substitution diethylphosphonoacetic acid butoxycarbonyl-ethylsulfanylcarbonyloxy-methyl ester for isobutyric acid ethoxycarbonyl-ethylsulfanylcarbonyloxy-methyl ester, using 1.9 equiv of sulfuryl chloride and starting the reaction at 5° C. ¹H-NMR (300 MHz, CDCl₃) δ (ppm): 0.96 (t, 3H), 1.37 (t, 6H), 1.72 (m, 2H), 2.01 (m, 2H), 3.15 (d, 2H), 4.27 (m, 6H), 6.85 (s, 1H).

### EXAMPLE 42

Pivalic acid butoxycarbonyl-chlorocarbonyloxy-methyl ester (Compound 4-11 of Table 4)

The title compound was prepared according to the procedure described in Example 32 above except for the substitution of pivalic acid butoxycarbonyl-ethylsulfanylcarbonyloxy-methyl ester for isobutyric acid ethoxycarbonyl-ethylsulfanylcarbonyloxy-methyl ester, using 1.9 equiv of sulfuryl chloride and starting the reaction at 5° C. ¹H-NMR (300 MHz, CDCl₃) 8 (ppm): 0.95 (t, 3H), 1.27 (s, 9H), 1.35-1.45 (m, 2H), 1.65-1.75 (m 2H), 4.31 (m, 2H), 6.85 (s, 1H).

### EXAMPLE 43

2-Methylpropanoic acid (chlorocarbonyloxy)-[(1-methylethoxy)carbonyl]methyl ester (Compound 4-12 of Table 4)

The title compound was prepared according to the procedure described in Example 32 above except for the substitution of isobutyric acid isobutoxycarbonyl-ethylsulfanylcarbonyloxy-methyl ester for isobutyric acid ethoxycarbonyl-ethylsulfanylcarbonyloxy-methyl ester, using 1.9 equiv of sulfuryl chloride and starting the reaction at 5° C. ¹H-NMR (300 MHz, CDCl₃), (ppm): 1.22-1.30 (m, 6H), 1.30-1.40 (m, 6H), 2.70 (heptet, 1H), 5.14 (heptet, 1H), 6.77 (s, 1H).

### EXAMPLE 44

2-Methylpropanoic acid (chlorocarbonyloxy)(butoxycarbonyl)methyl ester (Compound 4-13 of Table 4)

Diisopropylethylamine (0.57 g, 4.41 mmole) was added in three portions 2 min apart to a shaken solution of butyl 2-(ethylthiocarbonyloxy)-2-iodoethanoate (1.01 g, 2.92 mmol) and 2-methyl-2-propanoic acid (0.32 g, 3.63 mmole) in 1.11 g anhydrous tetrahydrofuran. One minute later, isobutyric acid (0.13 g, 1.47 mmole) was added to neutralize an accidental amine overcharge. After the mixture stood with occasional shaking for 16 h at room temperature, the slurry was concentrated in vacuo to 2.00 g, and the residue was extracted with three 3.4-5.5 g portions of ca. 2/1 w/w diethylether/hexanes. The supernatants were washed with 41.3 wt. % aqueous potassium carbonate solution and concentrated in vacuo to give 2-methylpropanoic acid (ethylthiocarbonyloxy)-(butoxycarbonyl)methyl ester (0.79 g, 2.58 mmol), as a pale yellow oil. This oil was dissolved in 1.09 g dichloromethane and 97% sulfuryl chloride (0.90 g, 6.67 mmol) was added in three portions 1 min apart at room temperature. After the resulting solution stood for another 68 min, it was concentrated in vacuo to constant weight to give 2-methylpropanoic acid (chlorocarbonyloxy)-(butoxycarbonyl)methyl ester (0.73 g, 2.60 mmole, 89% overall yield) as a yellow oil. ¹H-NMR (300 MHz, CDCl₃) 8 (ppm): 0.95 (t, 3H), 1.24 (d, 3H), 1.25 (d, 2H), 1.39 (sextet, 2H), 1.68 (p, 4H), 2.71 (septet, 1H), 6.99(s, 1H).

### EXAMPLE 45

Benzoic acid (chlorocarbonyloxy)(isopropoxycarbonyl)methyl ester (Compound 4-14 of Table 4)

Isopropyl 2-(ethylthiocarbonyloxy)-2-iodoethanoate (1.01 g, 3.04 mmol) was added to a well-mixed solution of benzoic acid (0.56 g, 4.59 mmol) and diisopropylethylamine (0.55 g, 4.25 mmol) in 2.20 g anhydrous tetrahydrofuran. This gave a slight exotherm and slow fading of the red color with precipitation of solid. After the mixture stood with occasional shaking for 4.3 h at room temperature, the slurry was concentrated in vacuo to 2.64 g and the residue was partitioned between 1.32 g deionized water containing 1.09 g 41.3% potassium carbonate solution and three 1.6-2.6 g hexanes extracts. The extracts were concentrated in vacuo to give benzoic acid (ethylthiocarbonyloxy)-(isopropoxycarbonyl)-methyl ester (0.87 g, 2.67 mmole), as a pale yellow oil. This oil was dissolved in 1.32 g dichloromethane and 97% sulfuryl chloride (0.89 g, 6.59 mmol) was added in three portions over 3 min at room temperature. The vigorous foaming soon subsided. After the resulting solution stood for another 2.4 h, it was concentrated in vacuo to constant weight to give benzoic acid (chlorocarbonyloxy)-(isopropoxycarbonyl)methyl ester (0.85 g, 2.83 mmole, 93% overall yield) as a yellow oil. ¹H-NMR (300 MHz, CDCl₃) δ (ppm): 1.34 (d, 6H), 5.20 (septet, 1H), 7.03 (s, 1H), 7.50 (t, 2H), 7.66 (t, H), 8.11 (d, 2H).

### EXAMPLE 46

Dodecanoic acid (chlorocarbonyloxy)(isopropoxycarbonyl)methyl ester (Compound 4-15 of Table 4)

Isopropyl 2-(ethylthiocarbonyloxy)-2-iodoethanoate (1.01 g, 3.04 mmol) was added to a well-mixed solution of dodecanoic acid (0.91 g, 4.54 mmol) and diisopropylethylamine (0.56 g, 4.33 mmol) in 2.12 g anhydrous tetrahydrofuran. This gave a slight exotherm and slow fading of the red color with precipitation of solid. After the mixture stood with occasional shaking for 21.7 h at room temperature, the slurry was concentrated in a vacuo to 2.9 g and the residue was extracted with four 2.8-3.9 g portions of hexanes. The extracts were washed with 0.94 g 41.3% potassium carbonate solution and concentrated in vacuo to give dodecanoic acid (ethylthiocarbonyloxy)(isopropoxycarbonyl)methyl ester (0.77 g, 1.90 mmol), as a pale yellow oil. This oil was dissolved in 1.42 g dichloromethane and 97% sulfuryl chloride (0.73 g, 5.41 mmol) was added in three portions over 9 min at room temperature. The vigorous foaming soon subsided. After the resulting solution stood for another 3.6 h, it was concentrated in vacuo to constant weight to give dodecanoic acid (chlorocarbonyloxy)-(isopropoxycarbonyl)methyl ester (0.75 g, 1.98 mmole, 65% overall yield) as a yellow oil. ¹H-NMR (300 MHz, CDCl₃) δ (ppm): 0.88 (t, 3H), 1.26 (bs, 16H), 1.31 (two equal doublets 1.3 Hz apart, 6H), 1.59 (p, 2H), 5.19 (septet, 1H), 6.78 (s, 1H).

### EXAMPLE 47

2-[4-(2-Methylpropyl)phenyl]propanoic acid (chlorocarbonyloxy)(isopropoxy-carbonyl)methyl ester (Compound 4-16 of Table 4)

Five 200 mg ibuprofen (α-methyl-4-(2-methylpropyl)benzeneacetic acid) tablets (total tablet wt. 1.64 g, 4.85 mmol) were stirred for 15 min with 3.56 g dichloromethane containing triisopropylamine (0.63 g, 4.87 mmol). Isopropyl 2-(ethylthiocarbonyloxy)-2-iodoethanoate (1.03 g, 3.10 mmol) was added with a 0.90 g dichloromethane pipet rinse, causing a slight exotherm and fading of a red color. After the mixture stood with occasional shaking for 28 h at room temperature, the slurry was concentrated in vacuo to 3.88 g and the residue was extracted with five 3.3-4.0 g portions of hexanes. The hexanes supernatants were washed with 41.3 wt. % aqueous potassium carbonate solution and concentrated in vacuo to give 2-[4-(2-methylpropyl)phenyl]propanoic acid (ethylthiocarbonyloxy)(isopropoxy-carbonyl)methyl ester (1.05 g, 2.56 mmol), as a pale yellow oil. This oil was dissolved in 1.52 g dichloromethane and 97% sulfuryl chloride (1.05 g, 7.78 mmol) was added in four portions over 5 min at room temperature. After the resulting solution stood for another 120 min, it was concentrated in vacuo to constant weight to give 2-[4-(2-methylpropyl)phenyl]propanoic acid (chloro-carbonyloxy)(isopropoxy-carbonyl)methyl ester (1.00 g, 84% overall yield) as a yellow oil. ¹H-NMR (300 MHz, CDCl₃) δ (ppm): 0.90 ppm (d, 6H), 1.14-1.20 (two d, total of 3H), 1.27 (d, 3H), 1.55 d (3H), 1.56 (septet, 1H), 1.84 (septet, 1H), 2.45 (dd, 2H), 3.82 and 3.84 (two equal q, total of 1H), 5.04 and 5.11 (two equal septets, total of 1H), 6.74 and 6.76 (two equal s, total of 1H), 7.11 (d, 2H), 7.20 (d, 2H).

Following Method A described hereinbefore, the following compound of Formula XX as listed in Table 5 was prepared.

TABLE 5

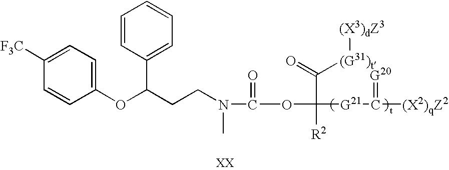

Listing of Compound of Formula XX

| Cmpd # | G²⁰ | G²¹ | G³¹ | R² | t′ | d | (X³)_{d}Z³ | t | q | (X²)_{q}Z² |
|---|---|---|---|---|---|---|---|---|---|---|
| 5-1 | O | O | O | H | 1 | 0 | 2- | 1 | 0 | 2- |
| | | | | | | | propyl | | | propyl |

The following Example is provided for guidance to the practitioner in order to practice the invention.

### EXAMPLE 48

2-Methylpropionic acid (N-methyl-3-phenyl-3-[(4-trifluoromethyl)phenoxy]propylcarbamoyloxy)-(2-propyloxycarbonyl)methyl ester (Compound 5-1 of Table 5)

2-Methylpropanoic acid (chlorocarbonyloxy)-[(1-methylethoxy)carbonyl]methyl ester (Compound 4-12, 28 μL, 0.12 mmol) was added via syringe to a solution of fluoxetine hydrochloride (Sigma Chemical Co., 42.5 mg, 0.123 mmol) and 4-(dimethylamino)pyridine (30.0 mg, 0.246 mmol) in THF (1.9 mL) at room temperature under N₂. The reaction was allowed to stir for 16 h. Then saturated aqueous ammonium chloride was added, the product was extracted into ethyl acetate, the extract was dried over Na₂SO₄, and the volatiles were removed in vacuo. The crude product was purified by flash chromatography on silica gel using a hexanes-ethyl acetate gradient. The title compound was isolated cleanly as a pale yellow oil in 74% yield and consisted of a mixture of diastereomers. ¹H-NMR (300 MHz, CDCl₃) δ (ppm): 1.14-1.28 (m, 12H), 2.10-2.32 (m, 2H), 3.53-3.75 (m, 1H), 2.93-2.95 (m, 3H), 3.51 (t, 2H), 5.05-5.17 (m, 1H), 5.17-5.27 (m, 1H), 6.70-6.75 (four singlets, 1H), 6.89 (d, 2H), 7.22-7.36 (m, 5H), 7.42 (d, 2H).

Following Method A described hereinbefore, the following compound of Formula XXII as listed in Table 6 was prepared.

TABLE 6

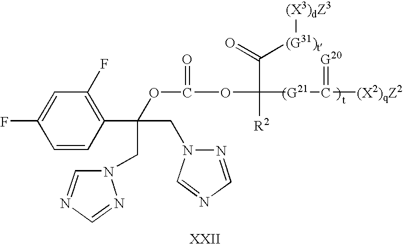

Listing of Compound of Formula XXII

| Cmpd # | G²⁰ | G²¹ | G³¹ | R² | t′ | d | (X³)_{d}Z³ | t | q | (X²)_{q}Z² |
|---|---|---|---|---|---|---|---|---|---|---|
| 6-1 | O | O | O | H | 1 | 0 | 2- | 1 | 0 | 2- |
| | | | | | | | propyl | | | propyl |

The following Example is provided for guidance to the practitioner in order to practice the invention.

### EXAMPLE 49

2-Methylpropionic acid [(2,4-difluoro-1-phenyl)-bis-(1H-1,2,4-triazol 1-ylmethyl)]methyloxycarbonyloxy-(2-propyloxycarbonyl)methyl ester (Compound 6-1 of Table 6)

Potassium bis(trimethylsilyl)amide (0.327 mL of a 0.5 M solution in toluene, 0.16 mmol) was added via syringe to a solution of fluconazole (Pfizer, 50.5 mg, 0.165 mmol) in THF (1.5 mL) at −78° C. under N₂. The reaction was allowed to stir at −78° C. for 10 min, then at 0° C. for 35 min, at which time 2-methylpropanoic acid (chlorocarbonyloxy)-[(1-methylethoxy)carbonyl]methyl ester (Compound 4-12, 76 μL, 0.16 mmol) was added via syringe. The reaction was allowed to warm slowly to room temperature over 22 h. Then saturated aqueous ammonium chloride was added, the product was extracted into ethyl acetate, the extract was dried over Na2SO₄, and the volatiles were removed in vacuo. The crude product was purified by flash chromatography on silica gel using an ethyl acetate-methanol gradient. The title compound was isolated as a pale yellow oil in 40% yield; unreacted fluconazole was also recovered (28%). ¹H-NMR (300 MHz, CDCl₃) δ (ppm): 1.22-1.25 (m, 6H), 1.28-1.31 (m, 6H), 2.70 (heptet, 1H), 5.06-5.24 (m, 5H), 6.73 (s, 1H), 6.73-6.81 (m, 1H), 6.82-6.96 (m, 2H), 7.85 (s, 1H), 7.86 (s, 1H), 8.08 (s, 1H), 8.12 (s, 1H).

Following Method A described hereinbefore, the following compound of Formula XXIV as listed in Table 7 was prepared.

TABLE 7

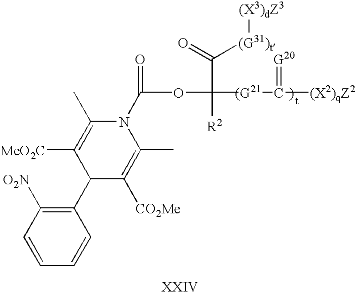

Listing of Compound of Formula XXIV

| Cmpd # | G²⁰ | G²¹ | G³¹ | R² | t′ | d | (X³)_{d}Z³ | t | q | (X²)_{q}Z² |
|---|---|---|---|---|---|---|---|---|---|---|
| 7-1 | O | O | O | H | 1 | 0 | 2- | 1 | 0 | 2- |
| | | | | | | | propyl | | | propyl |

The following Example is provided for guidance to the practitioner in order to practice the invention.

### EXAMPLE 50

2-Methylpropionic acid [2,6-Dimethyl-3,5-dicarbomethoxy-4-(2-nitrophenyl)-1,4-dihydropyridin-1-yl]carbonyloxy-(2-propyloxycarbonyl)methyl ester (Compound 7-1 of Table 7)

Potassium bis(trimethylsilyl)amide (0.296 mL of a 0.5 M solution in toluene, 0.148 mmol) was added via syringe to a solution of nifedipine (Sigma Chemical Co., 51.3 mg, 0.148 mmol) in THF (1.5 mL) at −78° C. under N₂. The reaction was allowed to stir at −78° C. for 15 min, then at 0° C. for 35 min, at which time 2-methylpropanoic acid (chlorocarbonyloxy)-[(1-methylethoxy)carbonyl]methyl ester (compound 4-12, 33 uL, 0.15 mmol) was added via syringe. The reaction was allowed to warm slowly to room temperature over 16 h. Then saturated aqueous ammonium chloride was added, the product was extracted into ethyl acetate, the extract was dried over Na₂SO₄, and the volatiles were removed under reduced pressure. The crude product was purified by flash chromatography on silica gel using a hexanes-ethyl acetate gradient. The title compound was isolated as a pale yellow oil in 12% yield; unreacted nifedipine was also recovered (54%). ¹H-NMR (300 MHz, CDCB) δ (ppm): 1.20-1.34 (m, 12H), 2.48 (s, 3H), 2.52 (s, 3H), 2.67 (heptet, 1H), 3.72 (s, 3H), 3.73 (s, 3H), 5.15 (heptet, 1H), 5.73 (s, 1H), 6.86 (s, 1H), 7.24-7.35 (m, 2H), 7.54 (t, 1H), 7.68 (d, 1H).

Following Method A described hereinbefore, the following compounds of Formula XXVI as listed in Table 8 were prepared.

TABLE 8

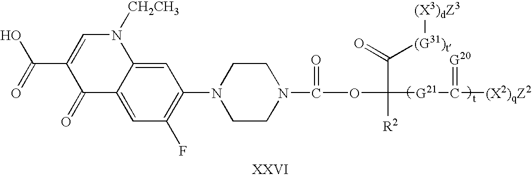

Listing of Compounds of Formula XXVI

| Cmpd # | G²⁰ | G²¹ | G³¹ | R² | t′ | d | (X³)_{d}Z³ | t | q | (X²)_{q}Z² |
|---|---|---|---|---|---|---|---|---|---|---|
| 8-1 | O | O | O | H | 1 | 0 | n-butyl | 1 | 0 | 2-propyl |
| 8-2 | O | O | O | H | 1 | 0 | 2-propyl | 1 | 0 | phenyl |
| 8-3 | O | O | O | H | 1 | 0 | n-butyl | 1 | 0 | undecyl |
| 8-4 | O | O | O | H | 1 | 0 | 2-propyl | 1 | 1 | 1-[4-(2-methyl- |
| | | | | | | | | | | propyl)phenyl]ethyl |

The following Examples are provided for guidance to the practitioner in order to practice the invention.

### EXAMPLE 51

7-(4-[(Butoxycarbonyl)(2-methylpropanoyloxy)methoxycarbonyl]-1-piperazinyl)-1-ethyl-6-fluoro-1,4-dihydro-4-oxo-3-quinolinecarboxylic acid (Compound 8-1 of Table 8)

2-Methylpropanoic acid (chlorocarbonyloxy)(butoxycarbonyl)methyl ester (0.22 g, 0.78 mmol) was added in two portions (0.19 g, 0.03 g) with shaking 2 min apart to a slurry of norfloxacin (0.22 g, 0.69 mmol) and triisopropylamine (0.15 g, 1.05 mmol) in 2.42 g CH₂Cl₂, causing a moderate exotherm and dissolution of much then almost all of the solid. The mixture stood another 10 min at room temperature before it was concentrated in vacuo to 0.78 g of gum and solid. This residue was repeatedly shaken with small portions of 3-4:1 w:w ethyl acetate:hexanes eluant which were added to the top of a 1.02 g (7.2 cm×0.65 cm ID) silica gel column. Evaporation of the eluate in 3 portions gave a total of 0.34 g of yellow to white solid crystals. The two colored portions of crystals were washed twice with diethyl ether/hexanes to give 0.05 g soluble gum and insoluble off-white crystals. The three portions of crystals were combined to give 7-(4-[(butoxycarbonyl)(2-methylpropanoyloxy)-methoxycarbonyl]-1-piperazinyl)-1-ethyl-6-fluoro-1,4-dihydro-4-oxo-3-quinoline-carboxylic acid (0.30 g, 77% yield). ¹H-NMR (300 MHz, CDCl₃) δ (ppm): 0.94 (t, 3H), 1.23 (d, 3H), 1.24 (d, 3H), 1.40 (sextet, 2H), 1.60 (t, 4H, area may include obscured water peak), 1.64 (p, 3H), 2.67 (septet, 1H), 3.31 (t, 4H), 3.77 (s, 4H), 4.24 (t, 2H), 4.33 (q, 2H), 6.85 (s, 1H), 6.86 (d, 1H)., 8.09 (d, 1H), and 8.68 (s, 1H). Mp=151.5-160.0° C. Reverse phase LC/electrospray MS gave a predominant LC peak with a positively charged P+1 ion at m/g 563.8 compared with a calculated m/g of 563.23 for the C₂₇H₃₄N₃O₉F parent ion of the assigned structure.

### EXAMPLE 52

7-(4-[(Isopropoxycarbonyl)(benzoyloxy)methoxycarbonyl]-1-piperazinyl)-1-ethyl-6-fluoro-1,4-dihydro-4-oxo-3-quinolinecarboxylic acid (Compound 8-2 of Table 8)

The title compound was prepared according to the procedure described in Example 51 above except for the substitution of benzoic acid (chlorocarbonyloxy)-(isopropoxycarbonyl)methyl ester for 2-methylpropanoic acid (chlorocarbonyl-oxy)(butoxycarbonyl)methyl ester. The yield of off-white crystals was 0.30 g (70% after correction for 7 wt. % ethyl acetate). Mp 135-143° C. Reverse phase LC/electrospray MS gave a predominant LC peak with a positively charged P+1 ion at m/g 583.7 compared with a calculated m/g of 583.20 for the C₂₉H₃₀N₃O₉F parent ion of the assigned structure.

### EXAMPLE 53

7-(4-[(Isopropoxycarbonyl)(dodecanoyloxy)methoxycarbonyl]-1-piperazinyl)-1-ethyl-6-fluoro-1,4-dihydro-4-oxo-3-quinolinecarboxylic acid (Compound 8-3 of Table 8)

The title compound was prepared according to the procedure described in Example 51 above except for the substitution of dodecanoic acid (chlorocarbonyl-oxy)(isopropoxycarbonyl)methyl ester for 2-methylpropanoic acid (chlorocarbonyloxy)(butoxycarbonyl)methyl ester. The yield of off-white crystals was 0.34 g (74%). Mp 105.5-109.2° C. Reverse phase LC/electrospray MS gave a predominant LC peak with a positively charged P+1 ion at m/g 661.8 compared with a calculated m/g of 661.34 for the C₃₄H₄₈N₃O₉F parent ion of the assigned structure.

### EXAMPLE 54

7-(4-[(2-[4-(2-Methylpropyl)phenyl]propanoyloxy)(isopropoxycarbonyl)methoxy-carbonyl]-1-piperazinyl)-1-ethyl-6-fluoro-1,4-dihydro-4-oxo-3-quinolinecarboxylic acid (Compound 8-4 of Table 8)

The title compound was prepared according to the procedure described in Example 51 above except for the substitution of 2-[4-(2-methylpropyl)phenyl]-propanoic acid (chlorocarbonyloxy)(isopropoxycarbonyl)methyl ester for 2-methylpropanoic acid (chlorocarbonyloxy)(butoxycarbonyl)methyl ester. The yield of off-white crystals was 0.41 g (89%). Mp 88.0-96.5° C. Reverse phase LC/electrospray MS gave a predominant LC peak with a positively charged P+1 ion at m/g 667.9 compared with a calculated m/g of 667.29 for the C₃₅H₄₂N₃O₉F parent ion of the assigned structure.

Following Method A described hereinbefore, the following compound of Formula XXVIII as listed in Table 9 was prepared.

TABLE 9

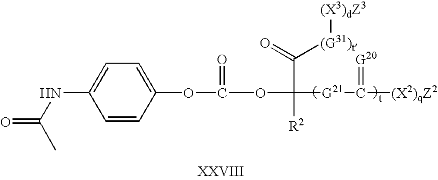

Listing of Compound of Formula XXVIII

| Cmpd # | G²⁰ | G²¹ | G³¹ | R² | t′ | d | (X³)_{d}Z³ | t | q | (X²)_{q}Z² |
|---|---|---|---|---|---|---|---|---|---|---|
| 9-1 | O | O | O | H | 1 | 0 | 2- | 1 | 0 | 2- |
| | | | | | | | propyl | | | propyl |

The following Example is provided for guidance to the practitioner in order to practice the invention.

### EXAMPLE 55

2-(2-Methylpropanoyloxy)-2-(4-acetamidophenoxycarbonyloxy)ethanoic acid 1-methylethyl ester (Compound 9-1 of Table 9)

4-Acetamidophenol (0.15 g, 0.99 mmol) was stirred for 8 min with 1.06 g absolute ethanol and fragmented 87.11 wt. % potassium hydroxide pellets (0.05 g, 0.78 meq) to form a homogeneous solution. Ethanol (0.96 g) was concentrated in vacuo, toluene (0.50 g) was added, and the mixture was concentrated in vacuo with repeated heating to give an additional 0.04 g weight loss. Dichloromethane (7.87 g) and 2-(chlorocarbonyloxy)-2-(2-methylpropanoyloxy)ethanoic acid 1-methylethyl ester (0.19 g, 0.71 mmol) were added with shaking without dissolving much of the gum. The mixture stood for 17 h at room temperature, causing solid to form in both the solvent and gum layers. The stirring bar was freed by breaking up the solid and the mixture was stirred for 3.3 h, forming a finely dispersed slurry. The slurry was concentrated in vacuo to a tan opaque gum which was extracted with four portions of ethyl acetate to separate crude (0.27 g, ca. 70% by NMR) 2-(2-methylpropanoyloxy)-2-(4-acetamidophenoxycarbonyloxy)ethanoic acid 1-methylethyl ester from insoluble solid. Short column silica gel chromatography using chloroform eluant gave purified 2-(2-methylpropanoyloxy)-2-(4-acetamidophenoxycarbonyloxy)-ethanoic acid 1-methylethyl ester (0.17 g, 63% yield) as a yellowish tan gum. Reverse phase LC/electrospray MS gave a predominant LC peak with a positively charged P+1 ion at m/g 381.7 compared with a calculated m/g of 381.14 for the C₁₈H₂₃NO₈ parent ion of the assigned structure.

Following Method A described hereinbefore, the following compound of Formula XXX as listed in Table 10 was prepared.

TABLE 10

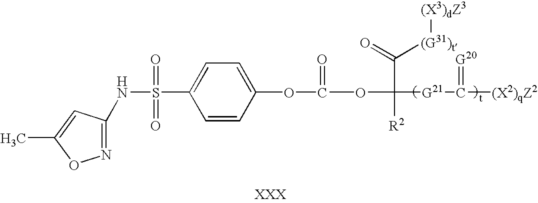

Listing of Compound of Formula XXX

| Cmpd # | G²⁰ | G²¹ | G³¹ | R² | t′ | d | (X³)_{d}Z³ | t | q | (X²)_{q}Z² |
|---|---|---|---|---|---|---|---|---|---|---|
| 10-1 | O | O | O | H | 1 | 0 | n-butyl | 1 | 0 | 2-propyl |

The following Example is provided for guidance to the practitioner in order to practice the invention.

### EXAMPLE 56

4-([(Butoxycarbonyl)(2-methylpropanoyloxy)methoxycarbonyl]amino)-N-(5-methyl-3-isoxazolyl)benzenesulfonamide (Compound 10-1 of Table 10)

2-Methylpropanoic acid (chlorocarbonyloxy)(butoxycarbonyl)methyl ester (0.21 g, 0.75 mmol) was added in two portions 2 min apart to a shaken slurry of 4-amino-N-(5-methyl-3-isoxazolyl)benzenesulfonamide (0.18 g, 0.71 mmol) in 1.51 g CH₂Cl₂ containing triisopropylamine (0.12 g, 0.84 mmol). This afforded a slightly delayed moderate exotherm and caused most of the solid to dissolve. After 10 min of cooling to room temperature, the mixture was concentrated in vacuo to 0.70 g, 0.67 g diethyl ether was added to induce crystallization, and the mixture was concentrated in vacuo to 0.65 g of partially crystalline gum. The mixture was repeatedly washed with small portions of diethyl ether, then with ethyl acetate/hexanes eluant and the supernatants were passed through a 1.14 g (7.72 cm×0.68 cm ID) silica gel column to give six fractions. Five of these fractions, totaling 0.35 g, appeared by NMR to contain significant amounts of the desired product. Normal phase preparative HPLC of the non-volatiles from the best of these fractions (0.13 g) gave high purity 4-([(butoxycarbonyl)(2-methyl-propanoyloxy)methoxycarbonyl]amino)-N-(5-methyl-3-isoxazolyl)benzene-sulfonamide (0.026 g, 7.3% yield) as a golden gum, along with an equal quantity of less pure material. Reverse phase LC/electrospray MS gave a predominant LC peak with a positively charged P+1 ion at m/g 497.7 compared with a calculated m/g of 497.15 for the C₂₁H₂₇N₃O₉S parent ion of the assigned structure.

Table A1 describes additional examples of compounds of Formula I which can be made using the procedures described hereinbefore, where R² is hydrogen, m=1, q=0, t=0 or 1 and the pharmaceutical which defines the pharmaceutical moiety of these examples is Z¹(X¹)ₘ—H. The following groups, Z¹(X¹), —H, X¹, G¹⁰, G¹¹, R¹, G²⁰, G²¹, t and Z² are defined within Table A1.

TABLE A1

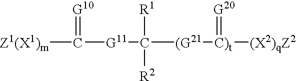

| Cmpd # | Z¹(X¹)ₘ—H | X¹ | G¹⁰ | G¹¹ | R¹ | G²⁰ | G²¹ | t | Z² |
|---|---|---|---|---|---|---|---|---|---|
| A1-1 | aletamine | N | O | O | CO₂H | O | O | 1 | methyl |
| A1-2 | aletamine | N | O | O | CO₂H | O | O | 1 | C(CH₃)₃ |
| A1-3 | aletamine | N | O | O | CO₂H | O | O | 1 | Ph |
| A1-4 | aletamine | N | O | O | CO₂H | O | O | 1 | 4-Me-Ph |
| A1-5 | aletamine | N | O | O | CO₂H | O | O | 1 | 2-pyridyl |
| A1-6 | aletamine | N | O | O | CO₂H | O | O | 1 | 4-pyridyl |
| A1-7 | aletamine | N | O | O | CO₂H | O | O | 1 | 2-furyl |
| A1-8 | aletamine | N | O | O | CO₂H | O | O | 1 | 2-thienyl |
| A1-9 | aletamine | N | O | O | CO₂H | — | — | 0 | Cl |
| A1-10 | aletamine | N | O | O | CO₂H | — | — | 0 | {N(Et)₃}⁺Cl⁻ |
| A1-11 | aletamine | N | O | O | CO₂H | O | O | 1 | CF₃CH₂ |
| A1-12 | aletamine | N | O | O | CO₂H | O | O | 1 | cyclopropyl |
| A1-13 | aletamine | N | O | O | CO₂H | O | O | 1 | 2-OMe-Ph |
| A1-14 | aletamine | N | O | O | CO₂H | O | O | 1 | MeSCH₂ |
| A1-15 | aletamine | N | O | O | CO₂H | O | O | 1 | MeOCH₂ |
| A1-16 | aletamine | N | O | O | CO₂H | S | S | 1 | Ph |
| A1-17 | aletamine | N | O | O | CO₂H | S | S | 1 | NMe₂ |
| A1-18 | aletamine | N | O | O | CO₂H | S | S | 1 | NBn₂ |
| A1-19 | aletamine | N | O | O | CO₂H | O | S | 1 | Ph |
| A1-20 | aletamine | N | O | O | CO₂H | O | S | 1 | Me |
| A1-21 | amphetamine | N | O | O | CO₂H | O | O | 1 | methyl |
| A1-22 | amphetamine | N | O | O | CO₂H | O | O | 1 | C(CH₃)₃ |
| A1-23 | amphetamine | N | O | O | CO₂H | O | O | 1 | Ph |
| A1-24 | amphetamine | N | O | O | CO₂H | O | O | 1 | 4-Me-Ph |
| A1-25 | amphetamine | N | O | O | CO₂H | O | O | 1 | 2-pyridyl |
| A1-26 | amphetamine | N | O | O | CO₂H | O | O | 1 | 4-pyridyl |
| A1-27 | amphetamine | N | O | O | CO₂H | O | O | 1 | 2-furyl |
| A1-28 | amphetamine | N | O | O | CO₂H | O | O | 1 | 2-thienyl |
| A1-29 | amphetamine | N | O | O | CO₂H | — | — | 0 | Cl |
| A1-30 | amphetamine | N | O | O | CO₂H | — | — | 0 | {N(Et)₃)⁺Cl⁻ |
| A1-31 | amphetamine | N | O | O | CO₂H | O | O | 1 | CF₃CH₂ |
| A1-32 | amphetamine | N | O | O | CO₂H | O | O | 1 | cyclopropyl |
| A1-33 | amphetamine | N | O | O | CO₂H | O | O | 1 | 2-OMe-Ph |
| A1-34 | amphetamine | N | O | O | CO₂H | O | O | 1 | MeSCH₂ |
| A1-35 | amphetamine | N | O | O | CO₂H | O | O | 1 | MeOCH₂ |
| A1-36 | amphetamine | N | O | O | CO₂H | S | S | 1 | Ph |
| A1-37 | amphetamine | N | O | O | CO₂H | S | S | 1 | NMe₂ |
| A1-38 | amphetamine | N | O | O | CO₂H | S | S | 1 | NBn₂ |
| A1-39 | amphetamine | N | O | O | CO₂H | O | S | 1 | Ph |
| A1-40 | amphetamine | N | O | O | CO₂H | O | S | 1 | Me |
| A1-41 | betahistine | N | O | O | CO₂H | O | O | 1 | methyl |
| A1-42 | betahistine | N | O | O | CO₂H | O | O | 1 | C(CH₃)₃ |
| A1-43 | betahistine | N | O | O | CO₂H | O | O | 1 | Ph |
| A1-44 | betahistine | N | O | O | CO₂H | O | O | 1 | 4-Me-Ph |
| A1-45 | betahistine | N | O | O | CO₂H | O | O | 1 | 2-pyridyl |
| A1-46 | betahistine | N | O | O | CO₂H | O | O | 1 | 4-pyridyl |
| A1-47 | betahistine | N | O | O | CO₂H | O | O | 1 | 2-furyl |
| A1-48 | betahistine | N | O | O | CO₂H | O | O | 1 | 2-thienyl |
| A1-49 | betahistine | N | O | O | CO₂H | — | — | 0 | Cl |
| A1-50 | betahistine | N | O | O | CO₂H | — | — | 0 | {N(Et)₃}⁺Cl⁻ |
| A1-51 | betahistine | N | O | O | CO₂H | O | O | 1 | CF₃CH₂ |
| A1-52 | betahistine | N | O | O | CO₂H | O | O | 1 | cyclopropyl |
| A1-53 | betahistine | N | O | O | CO₂H | O | O | 1 | 2-OMe-Ph |
| A1-54 | betahistine | N | O | O | CO₂H | O | O | 1 | MeSCH₂ |
| A1-55 | betahistine | N | O | O | CO₂H | O | O | 1 | MeOCH₂ |
| A1-56 | betahistine | N | O | O | CO₂H | S | S | 1 | Ph |
| A1-57 | betahistine | N | O | O | CO₂H | S | S | 1 | NMe₂ |
| A1-58 | betahistine | N | O | O | CO₂H | S | S | 1 | NBn₂ |
| A1-59 | betahistine | N | O | O | CO₂H | O | S | 1 | Ph |
| A1-60 | betahistine | N | O | O | CO₂H | O | S | 1 | Me |
| A1-61 | clonidine | N | O | O | CO₂H | O | O | 1 | methyl |
| A1-62 | clonidine | N | O | O | CO₂H | O | O | 1 | C(CH₃)₃ |
| A1-63 | clonidine | N | O | O | CO₂H | O | O | 1 | Ph |
| A1-64 | clonidine | N | O | O | CO₂H | O | O | 1 | 4-Me-Ph |
| A1-65 | clonidine | N | O | O | CO₂H | O | O | 1 | 2-pyridyl |
| A1-66 | clonidine | N | O | O | CO₂H | O | O | 1 | 4-pyridyl |
| A1-67 | clonidine | N | O | O | CO₂H | O | O | 1 | 2-furyl |
| A1-68 | clonidine | N | O | O | CO₂H | O | O | 1 | 2-thienyl |
| A1-69 | clonidine | N | O | O | CO₂H | — | — | 0 | Cl |
| A1-70 | clonidine | N | O | O | CO₂H | — | — | 0 | {N(Et)₃}⁺Cl⁻ |
| A1-71 | clonidine | N | O | O | CO₂H | O | O | 1 | CF₃CH₂ |
| A1-72 | clonidine | N | O | O | CO₂H | O | O | 1 | cyclopropyl |
| A1-73 | clonidine | N | O | O | CO₂H | O | O | 1 | 2-OMe-Ph |
| A1-74 | clonidine | N | O | O | CO₂H | O | O | 1 | MeSCH₂ |
| A1-75 | clonidine | N | O | O | CO₂H | O | O | 1 | MeOCH₂ |
| A1-76 | clonidine | N | O | O | CO₂H | S | S | 1 | Ph |
| A1-77 | clonidine | N | O | O | CO₂H | S | S | 1 | NMe₂ |
| A1-78 | clonidine | N | O | O | CO₂H | S | S | 1 | NBn₂ |
| A1-79 | clonidine | N | O | O | CO₂H | O | S | 1 | Ph |
| A1-80 | clonidine | N | O | O | CO₂H | O | S | 1 | Me |
| A1-81 | etintidine | N | O | O | CO₂H | O | O | 1 | methyl |
| A1-82 | etintidine | N | O | O | CO₂H | O | O | 1 | C(CH₃)₃ |
| A1-83 | etintidine | N | O | O | CO₂H | O | O | 1 | Ph |
| A1-84 | etintidine | N | O | O | CO₂H | O | O | 1 | 4-Me-Ph |
| A1-85 | etintidine | N | O | O | CO₂H | O | O | 1 | 2-pyridyl |
| A1-86 | etintidine | N | O | O | CO₂H | O | O | 1 | 4-pyridyl |
| A1-87 | etintidine | N | O | O | CO₂H | O | O | 1 | 2-furyl |
| A1-88 | etintidine | N | O | O | CO₂H | O | O | 1 | 2-thienyl |
| A1-89 | etintidine | N | O | O | CO₂H | — | — | 0 | Cl |
| A1-90 | etintidine | N | O | O | CO₂H | — | — | 0 | {N(Et)₃}⁺Cl⁻ |
| A1-91 | etintidine | N | O | O | CO₂H | O | O | 1 | CF₃CH₂ |
| A1-92 | etintidine | N | O | O | CO₂H | O | O | 1 | cyclopropyl |
| A1-93 | etintidine | N | O | O | CO₂H | O | O | 1 | 2-OMe-Ph |
| A1-94 | etintidine | N | O | O | CO₂H | O | O | 1 | MeSCH₂ |
| A1-95 | etintidine | N | O | O | CO₂H | O | O | 1 | MeOCH₂ |
| A1-96 | etintidine | N | O | O | CO₂H | S | S | 1 | Ph |
| A1-97 | etintidine | N | O | O | CO₂H | S | S | 1 | NMe₂ |
| A1-98 | etintidine | N | O | O | CO₂H | S | S | 1 | NBn₂ |
| A1-99 | etintidine | N | O | O | CO₂H | O | S | 1 | Ph |
| A1-100 | etintidine | N | O | O | CO₂H | O | S | 1 | Me |
| A1-101 | fenfluramine | N | O | O | CO₂H | O | O | 1 | methyl |
| A1-102 | fenfluramine | N | O | O | CO₂H | O | O | 1 | C(CH₃)₃ |
| A1-103 | fenfluramine | N | O | O | CO₂H | O | O | 1 | Ph |
| A1-104 | fenfluramine | N | O | O | CO₂H | O | O | 1 | 4-Me-Ph |
| A1-105 | fenfluramine | N | O | O | CO₂H | O | O | 1 | 2-pyridyl |
| A1-106 | fenfluramine | N | O | O | CO₂H | O | O | 1 | 4-pyridyl |
| A1-107 | fenfluramine | N | O | O | CO₂H | O | O | 1 | 2-furyl |
| A1-108 | fenfluramine | N | O | O | CO₂H | O | O | 1 | 2-thienyl |
| A1-109 | fenfluramine | N | O | O | CO₂H | — | — | 0 | Cl |
| A1-110 | fenfluramine | N | O | O | CO₂H | — | — | 0 | {N(Et)₃}⁺Cl⁻ |
| A1-111 | fenfluramine | N | O | O | CO₂H | O | O | 1 | CF₃CH₂ |
| A1-112 | fenfluramine | N | O | O | CO₂H | O | O | 1 | cyclopropyl |
| A1-113 | fenfluramine | N | O | O | CO₂H | O | O | 1 | 2-OMe-Ph |
| A1-114 | fenfluramine | N | O | O | CO₂H | O | O | 1 | MeSCH₂ |
| A1-115 | fenfluramine | N | O | O | CO₂H | O | O | 1 | MeOCH₂ |
| A1-116 | fenfluramine | N | O | O | CO₂H | S | S | 1 | Ph |
| A1-117 | fenfluramine | N | O | O | CO₂H | S | S | 1 | NMe₂ |
| A1-118 | fenfluramine | N | O | O | CO₂H | S | S | 1 | NBn₂ |
| A1-119 | fenfluramine | N | O | O | CO₂H | O | S | 1 | Ph |
| A1-120 | fenfluramine | N | O | O | CO₂H | O | S | 1 | Me |
| A1-121 | fludorex | N | O | O | CO₂H | O | O | 1 | methyl |
| A1-122 | fludorex | N | O | O | CO₂H | O | O | 1 | C(CH₃)₃ |
| A1-123 | fludorex | N | O | O | CO₂H | O | O | 1 | Ph |
| A1-124 | fludorex | N | O | O | CO₂H | O | O | 1 | 4-Me-Ph |
| A1-125 | fludorex | N | O | O | CO₂H | O | O | 1 | 2-pyridyl |
| A1-126 | fludorex | N | O | O | CO₂H | O | O | 1 | 4-pyridyl |
| A1-127 | fludorex | N | O | O | CO₂H | O | O | 1 | 2-furyl |
| A1-128 | fludorex | N | O | O | CO₂H | O | O | 1 | 2-thienyl |
| A1-129 | fludorex | N | O | O | CO₂H | — | — | 0 | Cl |
| A1-130 | fludorex | N | O | O | CO₂H | — | — | 0 | {N(Et)₃}⁺Cl⁻ |
| A1-131 | fludorex | N | O | O | CO₂H | O | O | 1 | CF₃CH₂ |
| A1-132 | fludorex | N | O | O | CO₂H | O | O | 1 | cyclopropyl |
| A1-133 | fludorex | N | O | O | CO₂H | O | O | 1 | 2-OMe-Ph |
| A1-134 | fludorex | N | O | O | CO₂H | O | O | 1 | MeSCH₂ |
| A1-135 | fludorex | N | O | O | CO₂H | O | O | 1 | MeOCH₂ |
| A1-136 | fludorex | N | O | O | CO₂H | S | S | 1 | Ph |
| A1-137 | fludorex | N | O | O | CO₂H | S | S | 1 | NMe₂ |
| A1-138 | fludorex | N | O | O | CO₂H | S | S | 1 | NBn₂ |
| A1-139 | fludorex | N | O | O | CO₂H | O | S | 1 | Ph |
| A1-140 | fludorex | N | O | O | CO₂H | O | S | 1 | Me |
| A1-141 | nifedipine | N | O | O | CO₂H | O | O | 1 | methyl |
| A1-142 | nifedipine | N | O | O | CO₂H | O | O | 1 | C(CH₃3)₃ |
| A1-143 | nifedipine | N | O | O | CO₂H | O | O | 1 | Ph |
| A1-144 | nifedipine | N | O | O | CO₂H | O | O | 1 | 4-Me-Ph |
| A1-145 | nifedipine | N | O | O | CO₂H | O | O | 1 | 2-pyridyl |
| A1-146 | nifedipine | N | O | O | CO₂H | O | O | 1 | 4-pyridyl |
| A1-147 | nifedipine | N | O | O | CO₂H | O | O | 1 | 2-furyl |
| A1-148 | nifedipine | N | O | O | CO₂H | O | O | 1 | 2-thienyl |
| A1-149 | nifedipine | N | O | O | CO₂H | — | — | 0 | Cl |
| A1-150 | nifedipine | N | O | O | CO₂H | — | — | 0 | {N(Et)₃}^{+Cl}⁻ |
| A1-151 | nifedipine | N | O | O | CO₂H | O | O | 1 | CF₃CH₂ |
| A1-152 | nifedipine | N | O | O | CO₂H | O | O | 1 | cyclopropyl |
| A1-153 | nifedipine | N | O | O | CO₂H | O | O | 1 | 2-OMe-Ph |
| A1-154 | nifedipine | N | O | O | CO₂H | O | O | 1 | MeSCH₂ |
| A1-155 | nifedipine | N | O | O | CO₂H | O | O | 1 | MeOCH₂ |
| A1-156 | nifedipine | N | O | O | CO₂H | S | S | 1 | Ph |
| A1-157 | nifedipine | N | O | O | CO₂H | S | S | 1 | NMe₂ |
| A1-158 | nifedipine | N | O | O | CO₂H | S | S | 1 | NBn₂ |
| A1-159 | nifedipine | N | O | O | CO₂H | O | S | 1 | Ph |
| A1-160 | nifedipine | N | O | O | CO₂H | O | S | 1 | Me |
| A1-161 | oxmetidine | N | O | O | CO₂H | O | O | 1 | methyl |
| A1-162 | oxmetidine | N | O | O | CO₂H | O | O | 1 | C(CH₃)₃ |
| A1-163 | oxmetidine | N | O | O | CO₂H | O | O | 1 | Ph |
| A1-164 | oxmetidine | N | O | O | CO₂H | O | O | 1 | 4-Me-Ph |
| A1-165 | oxmetidine | N | O | O | CO₂H | O | O | 1 | 2-pyridyl |
| A1-166 | oxmetidine | N | O | O | CO₂H | O | O | 1 | 4-pyridyl |
| A1-167 | oxmetidine | N | O | O | CO₂H | O | O | 1 | 2-furyl |
| A1-168 | oxmetidine | N | O | O | CO₂H | O | O | 1 | 2-thienyl |
| A1-169 | oxmetidine | N | O | O | CO₂H | — | — | 0 | Cl |
| A1-170 | oxmetidine | N | O | O | CO₂H | — | — | 0 | {N(Et)₃}⁺Cl⁻ |
| A1-171 | oxmetidine | N | O | O | CO₂H | O | O | 1 | CF₃CH₂ |
| A1-172 | oxmetidine | N | O | O | CO₂H | O | O | 1 | cyclopropyl |
| A1-173 | oxmetidine | N | O | O | CO₂H | O | O | 1 | 2-OMe-Ph |
| A1-174 | oxmetidine | N | O | O | CO₂H | O | O | 1 | MeSOH₂ |
| A1-175 | oxmetidine | N | O | O | CO₂H | O | O | 1 | MeOCH₂ |
| A1-176 | oxmetidine | N | O | O | CO₂H | S | S | 1 | Ph |
| A1-177 | oxmetidine | N | O | O | CO₂H | S | S | 1 | NMe₂ |
| A1-178 | oxmetidine | N | O | O | CO₂H | S | S | 1 | NBn₂ |
| A1-179 | oxmetidine | N | O | O | CO₂H | O | S | 1 | Ph |
| A1-180 | oxmetidine | N | O | O | CO₂H | O | S | 1 | Me |
| A1-181 | tetrahydrazoline | N | O | O | CO₂H | O | O | 1 | methyl |
| A1-182 | tetrahydrazoline | N | O | O | CO₂H | O | O | 1 | C(CH₃)₃ |
| A1-183 | tetrahydrazoline | N | O | O | CO₂H | O | O | 1 | Ph |
| A1-184 | tetrahydrazoline | N | O | O | CO₂H | O | O | 1 | 4-Me-Ph |
| A1-185 | tetrahydrazoline | N | O | O | CO₂H | O | O | 1 | 2-pyridyl |
| A1-186 | tetrahydrazoline | N | O | O | CO₂H | O | O | 1 | 4-pyridyl |
| A1-187 | tetrahydrazoline | N | O | O | CO₂H | O | O | 1 | 2-furyl |
| A1-188 | tetrahydrazoline | N | O | O | CO₂H | O | O | 1 | 2-thienyl |
| A1-189 | tetrahydrazoline | N | O | O | CO₂H | — | — | 0 | Cl |
| A1-190 | tetrahydrazoline | N | O | O | CO₂H | — | — | 0 | {N(Et)₃}⁺Cl⁻ |
| A1-191 | tetrahydrazoline | N | O | O | CO₂H | O | O | 1 | CF₃CH₂ |
| A1-192 | tetrahydrazoline | N | O | O | CO₂H | O | O | 1 | cyclopropyl |
| A1-193 | tetrahydrazoline | N | O | O | CO₂H | O | O | 1 | 2-OMe-Ph |
| A1-194 | tetrahydrazoline | N | O | O | CO₂H | O | O | 1 | MeSCH₂ |
| A1-195 | tetrahydrazoline | N | O | O | CO₂H | O | O | 1 | MeOCH₂ |
| A1-196 | tetrahydrazoline | N | O | O | CO₂H | S | S | 1 | Ph |
| A1-197 | tetrahydrazoline | N | O | O | CO₂H | S | S | 1 | NMe₂ |
| A1-198 | tetrahydrazoline | N | O | O | CO₂H | S | S | 1 | NBn₂ |
| A1-199 | tetrahydrazoline | N | O | O | CO₂H | O | S | 1 | Ph |
| A1-200 | tetrahydrazoline | N | O | O | CO₂H | O | S | 1 | Me |
| A1-201 | procaine | N | O | O | CO₂H | O | O | 1 | methyl |
| A1-202 | procaine | N | O | O | CO₂H | O | O | 1 | C(CH₃)₃ |
| A1-203 | procaine | N | O | O | CO₂H | O | O | 1 | Ph |
| A1-204 | procaine | N | O | O | CO₂H | O | O | 1 | 4-Me-Ph |
| A1-205 | procaine | N | O | O | CO₂H | O | O | 1 | 2-pyridyl |
| A1-206 | procaine | N | O | O | CO₂H | O | O | 1 | 4-pyridyl |
| A1-207 | procaine | N | O | O | CO₂H | O | O | 1 | 2-furyl |
| A1-208 | procaine | N | O | O | CO₂H | O | O | 1 | 2-thienyl |
| A1-209 | procaine | N | O | O | CO₂H | — | — | 0 | Cl |
| A1-210 | procaine | N | O | O | CO₂H | — | — | 0 | {N(Et)₃}⁺Cl⁻ |
| A1-211 | procaine | N | O | O | CO₂H | O | O | 1 | CF₃CH₂ |
| A1-212 | procaine | N | O | O | CO₂H | O | O | 1 | cyclopropyl |
| A1-213 | procaine | N | O | O | CO₂H | O | O | 1 | 2-OMe-Ph |
| A1-214 | procaine | N | O | O | CO₂H | O | O | 1 | MeSCH₂ |
| A1-215 | procaine | N | O | O | CO₂H | O | O | 1 | MeOCH₂ |
| A1-216 | procaine | N | O | O | CO₂H | S | S | 1 | Ph |
| A1-217 | procaine | N | O | O | CO₂H | S | S | 1 | NMe₂ |
| A1-218 | procaine | N | O | O | CO₂H | S | S | 1 | NBn₂ |
| A1-219 | procaine | N | O | O | CO₂H | O | S | 1 | Ph |
| A1-220 | procaine | N | O | O | CO₂H | O | S | 1 | Me |
| A1-221 | thiabendazole | N | O | O | CO₂H | O | O | 1 | methyl |
| A1-222 | thiabendazole | N | O | O | CO₂H | O | O | 1 | C(CH₃)₃ |
| A1-223 | thiabendazole | N | O | O | CO₂H | O | O | 1 | Ph |
| A1-224 | thiabendazole | N | O | O | CO₂H | O | O | 1 | 4-Me-Ph |
| A1-225 | thiabendazole | N | O | O | CO₂H | O | O | 1 | 2-pyridyl |
| A1-226 | thiabendazole | N | O | O | CO₂E | O | O | 1 | 4-pyridyl |
| A1-227 | thiabendazole | N | O | O | CO₂H | O | O | 1 | 2-furyl |
| A1-228 | thiabendazole | N | O | O | CO₂H | O | O | 1 | 2-thienyl |
| A1-229 | thiabendazole | N | O | O | CO₂H | — | — | 0 | Cl |
| A1-230 | thiabendazole | N | O | O | CO₂H | — | — | 0 | {N(Et)₃}⁺Cl⁻ |
| A1-231 | thiabendazole | N | O | O | CO₂H | O | O | 1 | CF₃CH₂ |
| A1-232 | thiabendazole | N | O | O | CO₂H | O | O | 1 | cyclopropyl |
| A1-233 | thiabendazole | N | O | O | CO₂H | O | O | 1 | 2-OMe-Ph |
| A1-234 | thiabendazole | N | O | O | CO₂H | O | O | 1 | MeSCH₂ |
| A1-235 | thiabendazole | N | O | O | CO₂H | O | O | 1 | MeOCH₂ |
| A1-236 | thiabendazole | N | O | O | CO₂H | S | S | 1 | Ph |
| A1-237 | thiabendazole | N | O | O | CO₂H | S | S | 1 | NMe₂ |
| A1-238 | thiabendazole | N | O | O | CO₂H | S | S | 1 | NBn₂ |
| A1-239 | thiabendazole | N | O | O | CO₂H | O | S | 1 | Ph |
| A1-240 | thiabendazole | N | O | O | CO₂H | O | S | 1 | Me |
| A1-241 | tolazoline | N | O | O | CO₂H | O | O | 1 | methyl |
| A1-242 | tolazoline | N | O | O | CO₂H | O | O | 1 | C(CH₃)₃ |
| A1-243 | tolazoline | N | O | O | CO₂H | O | O | 1 | Ph |
| A1-244 | tolazoline | N | O | O | CO₂H | O | O | 1 | 4-Me-Ph |
| A1-245 | tolazoline | N | O | O | CO₂H | O | O | 1 | 2-pyridyl |
| A1-246 | tolazoline | N | O | O | CO₂H | O | O | 1 | 4-pyridyl |
| A1-247 | tolazoline | N | O | O | CO₂H | O | O | 1 | 2-furyl |
| A1-248 | tolazoline | N | O | O | CO₂H | O | O | 1 | 2-thienyl |
| A1-249 | tolazoline | N | O | O | CO₂H | — | — | 0 | Cl |
| A1-250 | tolazoline | N | O | O | CO₂H | — | — | 0 | {N(Et)₃}⁺Cl⁻ |
| A1-251 | tolazoline | N | O | O | CO₂H | O | O | 1 | CF₃CH₂ |
| A1-252 | tolazoline | N | O | O | CO₂H | O | O | 1 | cyclopropyl |
| A1-253 | tolazoline | N | O | O | CO₂H | O | O | 1 | 2-OMe-Ph |
| A1-254 | tolazoline | N | O | O | CO₂H | O | O | 1 | MeSCH₂ |
| A1-255 | tolazoline | N | O | O | CO₂H | O | O | 1 | MeOCH₂ |
| A1-256 | tolazoline | N | O | O | CO₂H | S | S | 1 | Ph |
| A1-257 | tolazoline | N | O | O | CO₂H | S | S | 1 | NMe₂ |
| A1-258 | tolazoline | N | O | O | CO₂H | S | S | 1 | NBn₂ |
| A1-259 | tolazoline | N | O | O | CO₂H | O | S | 1 | Ph |
| A1-260 | tolazoline | N | O | O | CO₂H | O | S | 1 | Me |
| A1-261 | proparacaine | N | O | O | CO₂H | O | O | 1 | methyl |
| A1-262 | proparacaine | N | O | O | CO₂H | O | O | 1 | C(CH₃)₃ |
| A1-263 | proparacaine | N | O | O | CO₂H | O | O | 1 | Ph |
| A1-264 | proparacaine | N | O | O | CO₂H | O | O | 1 | 4-Me-Ph |
| A1-265 | proparacaine | N | O | O | CO₂H | O | O | 1 | 2-pyridyl |
| A1-266 | proparacaine | N | O | O | CO₂H | O | O | 1 | 4-pyridyl |
| A1-267 | proparacaine | N | O | O | CO₂H | O | O | 1 | 2-furyl |
| A1-268 | proparacaine | N | O | O | CO₂H | O | O | 1 | 2-thienyl |
| A1-269 | proparacaine | N | O | O | CO₂H | — | — | 0 | Cl |
| A1-270 | proparacaine | N | O | O | CO₂H | — | — | 0 | {N(Et)₃}⁺Cl⁻ |
| A1-271 | proparacaine | N | O | O | CO₂H | O | O | 1 | CF₃CH₂ |
| A1-272 | proparacaine | N | O | O | CO₂H | O | O | 1 | cyclopropyl |
| A1-273 | proparacaine | N | O | O | CO₂H | O | O | 1 | 2-OMe-Ph |
| A1-274 | proparacaine | N | O | O | CO₂H | O | O | 1 | MeSCH₂ |
| A1-275 | proparacaine | N | O | O | CO₂H | O | O | 1 | MeOCH₂ |
| A1-276 | proparacaine | N | O | O | CO₂H | S | S | 1 | Ph |
| A1-277 | proparacaine | N | O | O | CO₂H | S | S | 1 | NMe₂ |
| A1-278 | proparacaine | N | O | O | CO₂H | S | S | 1 | NBn₂ |
| A1-279 | proparacaine | N | O | O | CO₂H | O | S | 1 | Ph |
| A1-280 | proparacaine | N | O | O | CO₂H | O | S | 1 | Me |
| A1-281 | tocainide | N | O | O | CO₂H | O | O | 1 | methyl |
| A1-282 | tocainide | N | O | O | CO₂H | O | O | 1 | C(CH₃)₃ |
| A1-283 | tocainide | N | O | O | CO₂H | O | O | 1 | Ph |
| A1-284 | tocainide | N | O | O | CO₂H | O | O | 1 | 4-Me-Ph |
| A1-285 | tocainide | N | O | O | CO₂H | O | O | 1 | 2-pyridyl |
| A1-286 | tocainide | N | O | O | CO₂H | O | O | 1 | 4-pyridyl |
| A1-287 | tocainide | N | O | O | CO₂H | O | O | 1 | 2-furyl |
| A1-288 | tocainide | N | O | O | CO₂H | O | O | 1 | 2-thienyl |
| A1-289 | tocainide | N | O | O | CO₂H | — | — | 0 | Cl |
| A1-290 | tocainide | N | O | O | CO₂H | — | — | 0 | {N(Et)₃}⁺Cl⁻ |
| A1-291 | tocainide | N | O | O | CO₂H | O | O | 1 | CF₃CH₂ |
| A1-292 | tocainide | N | O | O | CO₂H | O | O | 1 | cyclopropyl |
| A1-293 | tocainide | N | O | O | CO₂H | O | O | 1 | 2-OMe-Ph |
| A1-294 | tocainide | N | O | O | CO₂H | O | O | 1 | MeSCH₂ |
| A1-295 | tocainide | N | O | O | CO₂H | O | O | 1 | MeOCH₂ |
| A1-296 | tocainide | N | O | O | CO₂H | S | S | 1 | Ph |
| A1-297 | tocainide | N | O | O | CO₂H | S | S | 1 | NMe₂ |
| A1-298 | tocainide | N | O | O | CO₂H | S | S | 1 | NBn₂ |
| A1-299 | tacainide | N | O | O | CO₂H | O | S | 1 | Ph |
| A1-300 | tocainide | N | O | O | CO₂H | O | S | 1 | Me |
| A1-301 | triamterene | N | O | O | CO₂H | O | O | 1 | methyl |
| A1-302 | triamterene | N | O | O | CO₂H | O | O | 1 | C(CH₃)₃ |
| A1-303 | triamterene | N | O | O | CO₂H | O | O | 1 | Ph |
| A1-304 | triamterene | N | O | O | CO₂H | O | O | 1 | 4-Me-Ph |
| A1-305 | triamterene | N | O | O | CO₂H | O | O | 1 | 2-pyridyl |
| A1-306 | triamterene | N | O | O | CO₂H | O | O | 1 | 4-pyridyl |
| A1-307 | triamterene | N | O | O | CO₂H | O | O | 1 | 2-furyl |
| A1-308 | triarnterene | N | O | O | CO₂H | O | O | 1 | 2-thienyl |
| A1-309 | triamterene | N | O | O | CO₂H | — | — | 0 | Cl |
| A1-310 | triamterene | N | O | O | CO₂H | — | — | 0 | {N(Et)₃}⁺Cl⁻ |
| A1-311 | triamterene | N | O | O | CO₂H | O | O | 1 | CF₃CH₂ |
| A1-312 | triamterene | N | O | O | CO₂H | O | O | 1 | cyclopropyl |
| A1-313 | triamterene | N | O | O | CO₂H | O | O | 1 | 2-OMe-Ph |
| A1-314 | triamterene | N | O | O | CO₂H | O | O | 1 | MeSCH₂ |
| A1-315 | triamterene | N | O | O | CO₂H | O | O | 1 | MeOCH₂ |
| A1-316 | triamterene | N | O | O | CO₂H | S | S | 1 | Ph |
| A1-317 | triamterene | N | O | O | CO₂H | S | S | 1 | NMe₂ |
| A1-318 | triamterene | N | O | O | CO₂H | S | S | 1 | NBn₂ |
| A1-319 | triamterene | N | O | O | CO₂H | O | S | 1 | Ph |
| A1-320 | triamterene | N | O | O | CO₂H | O | S | 1 | Me |
| A1-321 | bydroxyitraconazole | O | O | O | CO₂H | O | O | 1 | methyl |
| A1-322 | hydroxyitraconazole | O | O | O | CO₂H | O | O | 1 | C(CH₃)₃ |
| A1-323 | hydroxyitraconazole | O | O | O | CO₂H | O | O | 1 | Ph |
| A1-324 | hydroxyitraconazole | O | O | O | CO₂H | O | O | 1 | 4-Me-Ph |
| A1-325 | hydroxyitraconazole | O | O | O | CO₂H | O | O | 1 | 2-pyridyl |
| A1-326 | hydroxyitraconazole | O | O | O | CO₂H | O | O | 1 | 4-pyridyl |
| A1-327 | hydroxyitraconazole | O | O | O | CO₂H | O | O | 1 | 2-furyl |
| A1-328 | hydroxyitraconazole | O | O | O | CO₂H | O | O | 1 | 2-thienyl |
| A1-329 | hydroxyitraconazole | O | O | O | CO₂H | — | — | O | Cl |
| A1-330 | hydroxyitraconazole | O | O | O | CO₂H | — | — | 0 | {N(Et)₃}⁺Cl⁻ |
| A1-331 | hydroxyitraconazole | O | O | O | CO₂H | O | O | 1 | CF₃CH₂ |
| A1-332 | hydroxyitraconazole | O | O | O | CO₂H | O | O | 1 | cyclopropyl |
| A1-333 | hydroxyitraconazole | O | O | O | CO₂H | O | O | 1 | 2-OMe-Ph |
| A1-334 | hydroxyitraconazole | O | O | O | CO₂H | O | O | 1 | MeSCH₂ |
| A1-335 | hydroxyitraconazole | O | O | O | CO₂H | O | O | 1 | MeOCH₂ |
| A1-336 | hydroxyitraconazole | O | O | O | CO₂H | S | S | 1 | Ph |
| A1-337 | hydroxyitraconazole | O | O | O | CO₂H | S | S | 1 | NMe₂ |
| A1-338 | hydroxyitraconazole | O | O | O | CO₂H | S | S | 1 | NBn₂ |
| A1-339 | hydroxyitraconazole | O | O | O | CO₂H | O | S | 1 | Ph |
| A1-340 | hydroxyitraconazole | O | O | O | CO₂H | O | S | 1 | Me |
| A1-341 | posaconazole | O | O | O | CO₂H | O | O | 1 | methyl |
| A1-342 | posaconazole | O | O | O | CO₂H | O | O | 1 | C(CH₃)₃ |
| A1-343 | posaconazole | O | O | O | CO₂H | O | O | 1 | Ph |
| A1-344 | posaconazole | O | O | O | CO₂H | O | O | 1 | 4-Me-Ph |
| A1-345 | posaconazole | O | O | O | CO₂H | O | O | 1 | 2-pyridyl |
| A1-346 | posaconazole | O | O | O | CO₂H | O | O | 1 | 4-pyridyl |
| A1-347 | posaconazole | O | O | O | CO₂H | O | O | 1 | 2-furyl |
| A1-348 | posaconazole | O | O | O | CO₂H | O | O | 1 | 2-thienyl |
| A1-349 | posaconazole | O | O | O | CO₂H | — | — | 0 | Cl |
| A1-350 | posaconazole | O | O | O | CO₂H | — | — | 0 | {N(Et)₃}⁺Cl⁻ |
| A1-351 | posaconazole | O | O | O | CO₂H | O | O | 1 | CF₃CH₂ |
| A1-352 | posaconazole | O | O | O | CO₂H | O | O | 1 | cyclopropyl |
| A1-353 | posaconazole | O | O | O | CO₂H | O | O | 1 | 2-OMe-Ph |
| A1-354 | posaconazole | O | O | O | CO₂H | O | O | 1 | MeSCH₂ |
| A1-355 | posaconazole | O | O | O | CO₂H | O | O | 1 | MeOCH₂ |
| A1-356 | posaconazole | O | O | O | CO₂H | S | S | 1 | Ph |
| A1-357 | posaconazole | O | O | O | CO₂H | S | S | 1 | NMe₂ |
| A1-358 | posaconazole | O | O | O | CO₂H | S | S | 1 | NBn₂ |
| A1-359 | posaconazole | O | O | O | CO₂H | O | S | 1 | Ph |
| A1-360 | posaconazole | O | O | O | CO₂H | O | S | 1 | Me |
| A1-361 | voriconazole | O | O | O | CO₂H | O | O | 1 | methyl |
| A1-362 | voriconazole | O | O | O | CO₂H | O | O | 1 | C(CH₃)₃ |
| A1-363 | voriconazole | O | O | O | CO₂H | O | O | 1 | Ph |
| A1-364 | voriconazole | O | O | O | CO₂H | O | O | 1 | 4-Me-Ph |
| A1-365 | voriconazole | O | O | O | CO₂H | O | O | 1 | 2-pyridyl |
| A1-366 | voriconazole | O | O | O | CO₂H | O | O | 1 | 4-pyridyl |
| A1-367 | voriconazole | O | O | O | CO₂H | O | O | 1 | 2-furyl |
| A1-368 | voriconazole | O | O | O | CO₂H | O | O | 1 | 2-thienyl |
| A1-369 | voriconazole | O | O | O | CO₂H | — | — | 0 | Cl |
| A1-370 | voriconazole | O | O | O | CO₂H | — | — | 0 | {N(Et)₃}⁺Cl⁻ |
| A1-371 | voriconazole | O | O | O | CO₂H | O | O | 1 | CF₃CH₂ |
| A1-372 | voriconazole | O | O | O | CO₂H | O | O | 1 | cyclopropyl |
| A1-373 | voriconazole | O | O | O | CO₂H | O | O | 1 | 2-OMe-Ph |
| A1-374 | voriconazole | O | O | O | CO₂H | O | O | 1 | MeSCH₂ |
| A1-375 | voriconazole | O | O | O | CO₂H | O | O | 1 | MeOCH₂ |
| A1-376 | voriconazole | O | O | O | CO₂H | S | S | 1 | Ph |
| A1-377 | voriconazole | O | O | O | CO₂H | S | S | 1 | NMe₂ |
| A1-378 | voriconazole | O | O | O | CO₂H | S | S | 1 | NBn₂ |
| A1-379 | voriconazole | O | O | O | CO₂H | O | S | 1 | Ph |
| A1-380 | voriconazole | O | O | O | CO₂H | O | S | 1 | Me |
| A1-381 | genaconazole | O | O | O | CO₂H | O | O | 1 | methyl |
| A1-382 | genaconazole | O | O | O | CO₂H | O | O | 1 | C(CH₃)₃ |
| A1-383 | genaconazole | O | O | O | CO₂H | O | O | 1 | Ph |
| A1-384 | genaconazole | O | O | O | CO₂H | O | O | 1 | 4-Me-Ph |
| A1-385 | genaconazole | O | O | O | CO₂H | O | O | 1 | 2-pyridyl |
| A1-386 | genaconazole | O | O | O | CO₂H | O | O | 1 | 4-pyridyl |
| A1-387 | genaconazole | O | O | O | CO₂H | O | O | 1 | 2-furyl |
| A1-388 | genaconazole | O | O | O | CO₂H | O | O | 1 | 2-thienyl |
| A1-389 | genaconazole | O | O | O | CO₂H | — | — | 0 | Cl |
| A1-390 | genaconazole | O | O | O | CO₂H | — | — | 0 | {N(Et)₃}⁺Cl⁻ |
| A1-391 | genaconazole | O | O | O | CO₂H | O | O | 1 | CF₃CH₂ |
| A1-392 | genaconazole | O | O | O | CO₂H | O | O | 1 | cyclopropyl |
| A1-393 | genaconazole | O | O | O | CO₂H | O | O | 1 | 2-OMe-Ph |
| A1-394 | genaconazole | O | O | O | CO₂H | O | O | 1 | MeSCH₂ |
| A1-395 | genaconazole | O | O | O | CO₂H | O | O | 1 | MeOCH₂ |
| A1-396 | genaconazole | O | O | O | CO₂H | S | S | 1 | Ph |
| A1-397 | genaconazole | O | O | O | CO₂H | S | S | 1 | NMe₂ |
| A1-398 | genaconazole | O | O | O | CO₂H | S | S | 1 | NBn₂ |
| A1-399 | genaconazole | O | O | O | CO₂H | O | S | 1 | Ph |
| A1-400 | genaconazole | O | O | O | CO₂H | O | S | 1 | Me |
| A1-401 | fluconazole | O | O | O | CO₂H | O | O | 1 | methyl |
| A1-402 | fluconazole | O | O | O | CO₂H | O | O | 1 | C(CH₃)₃ |
| A1-403 | fluconazole | O | O | O | CO₂H | O | O | 1 | Ph |
| A1-404 | fluconazole | O | O | O | CO₂H | O | O | 1 | 4-Me-Ph |
| A1-405 | fluconazole | O | O | O | CO₂H | O | O | 1 | 2-pyridyl |
| A1-406 | fluconazole | O | O | O | CO₂H | O | O | 1 | 4-pyridyl |
| A1-407 | fluconazole | O | O | O | CO₂H | O | O | 1 | 2-furyl |
| A1-408 | fluconazole | O | O | O | CO₂H | O | O | 1 | 2-thienyl |
| A1-409 | fluconazole | O | O | O | CO₂H | — | — | 0 | Cl |
| A1-410 | fluconazole | O | O | O | CO₂H | — | — | 0 | {N(Et)₃}⁺Cl⁻ |
| A1-411 | fluconazole | O | O | O | CO₂H | O | O | 1 | CF₃CH₂ |
| A1-412 | fluconazole | O | O | O | CO₂H | O | O | 1 | cyclopropyl |
| A1-413 | fluconazole | O | O | O | CO₂H | O | O | 1 | 2-OMe-Ph |
| A1-414 | fluconazole | O | O | O | CO₂H | O | O | 1 | MeSCH₂ |
| A1-415 | fluconazole | O | O | O | CO₂H | O | O | 1 | MeOCH₂ |
| A1-416 | fluconazole | O | O | O | CO₂H | S | S | 1 | Ph |
| A1-417 | fluconazole | O | O | O | CO₂H | S | S | 1 | NMe₂ |
| A1.418 | fluconazole | O | O | O | CO₂H | S | S | 1 | NBn₂ |
| A1-419 | fluconazole | O | O | O | CO₂H | O | S | 1 | Ph |
| A1-420 | fluconazole | O | O | O | CO₂H | O | S | 1 | Me |
| A1-421 | metronidazole | O | O | O | CO₂H | O | O | 1 | methyl |
| A1-422 | metronidazole | O | O | O | CO₂H | O | O | 1 | C(CH₃)₃ |
| A1-423 | metronidazole | O | O | O | CO₂H | O | O | 1 | Ph |
| A1-424 | metronidazole | O | O | O | CO₂H | O | O | 1 | 4-Me-Ph |
| A1-425 | metronidazole | O | O | O | CO₂H | O | O | 1 | 2-pyridyl |
| A1-426 | metronidazole | O | O | O | CO₂H | O | O | 1 | 4-pyridyl |
| A1-427 | metronidazole | O | O | O | CO₂H | O | O | 1 | 2-furyl |
| A1-428 | metronidazole | O | O | O | CO₂H | O | O | 1 | 2-thienyl |
| A1-429 | metronidazole | O | O | O | CO₂H | — | — | 0 | Cl |
| A1-430 | metronidazole | O | O | O | CO₂H | — | — | 0 | {N(Et)₃}⁺Cl⁻ |
| A1-431 | metronidazole | O | O | O | CO₂H | O | O | 1 | CF₃CH₂ |
| A1-432 | metronidazole | O | O | O | CO₂H | O | O | 1 | cyclopropyl |
| A1-433 | metronidazole | O | O | O | CO₂H | O | O | 1 | 2-OMe-Ph |
| A1-434 | metronidazole | O | O | O | CO₂H | O | O | 1 | MeSOH₂ |
| A1-435 | metronidazole | O | O | O | CO₂H | O | O | 1 | MeOCH₂ |
| A1-436 | metronidazole | O | O | O | CO₂H | S | S | 1 | Ph |
| A1-437 | metronidazole | O | O | O | CO₂H | S | S | 1 | NMe₂ |
| A1-438 | metronidazole | O | O | O | CO₂H | S | S | 1 | NBn₂ |
| A1-439 | metronidazole | O | O | O | CO₂H | O | S | 1 | Ph |
| A1-440 | metronidazole | O | O | O | CO₂H | O | S | 1 | Me |
| A1-441 | temazepam | O | O | O | CO₂H | O | O | 1 | methyl |
| A1-442 | temazepam | O | O | O | CO₂H | O | O | 1 | C(CH₃)₃ |
| A1-443 | temazepam | O | O | O | CO₂H | O | O | 1 | Ph |
| A1-444 | temazepam | O | O | O | CO₂H | O | O | 1 | 4-Me-Ph |
| A1-445 | temazepam | O | O | O | CO₂H | O | O | 1 | 2-pyridyl |
| A1-446 | temazepam | O | O | O | CO₂H | O | O | 1 | 4-pyridyl |
| A1-447 | temazepam | O | O | O | CO₂H | O | O | 1 | 2-furyl |
| A1-448 | temazepam | O | O | O | CO₂H | O | O | 1 | 2-thienyl |
| A1-449 | temazepam | O | O | O | CO₂H | — | — | 0 | Cl |
| A1-450 | temazepam | O | O | O | CO₂H | — | — | 0 | {N(Et)₃}⁺Cl⁻ |
| A1-451 | temazepam | O | O | O | CO₂H | O | O | 1 | CF₃CH₂ |
| A1-452 | temazepam | O | O | O | CO₂H | O | O | 1 | cyclopropyl |
| A1-453 | temazepam | O | O | O | CO₂H | O | O | 1 | 2-OMe-Ph |
| A1-454 | temazepam | O | O | O | CO₂H | O | O | 1 | MeSCH₂ |
| A1-455 | temazepam | O | O | O | CO₂H | O | O | 1 | MeOCH₂ |
| A1-456 | temazepam | O | O | O | CO₂H | S | S | 1 | Ph |
| A1-457 | temazepam | O | O | O | CO₂H | S | S | 1 | NMe₂ |
| A1-458 | temazepam | O | O | O | CO₂H | S | S | 1 | NBn₂ |
| A1-459 | temazepam | O | O | O | CO₂H | O | S | 1 | Ph |
| A1-460 | temazepam | O | O | O | CO₂H | O | S | 1 | Me |
| A1-461 | buprenorphine | O | O | O | CO₂H | O | O | 1 | methyl |
| A1-462 | buprenorphine | O | O | O | CO₂H | O | O | 1 | C(CH₃)₃ |
| A1-463 | buprenorphine | O | O | O | CO₂H | O | O | 1 | Ph |
| A1-464 | buprenorphine | O | O | O | CO₂H | O | O | 1 | 4-Me-Ph |
| A1-465 | buprenorphine | O | O | O | CO₂H | O | O | 1 | 2-pyridyl |
| A1-466 | buprenorphine | O | O | O | CO₂H | O | O | 1 | 4-pyridyl |
| A1-467 | buprenorphine | O | O | O | CO₂H | O | O | 1 | 2-furyl |
| A1-468 | buprenorphine | O | O | O | CO₂H | O | O | 1 | 2-thienyl |
| A1-469 | buprenorphine | O | O | O | CO₂H | — | — | 0 | Cl |
| A1-470 | buprenorphine | O | O | O | CO₂H | — | — | 0 | {N(Et)₃}⁺Cl⁻ |
| A1-471 | buprenorphine | O | O | O | CO₂H | O | O | 1 | CF₃CH₂ |
| A1-472 | buprenorphine | O | O | O | CO₂H | O | O | 1 | cyclopropyl |
| A1-473 | buprenorphine | O | O | O | CO₂H | O | O | 1 | 2-OMe-Ph |
| A1-474 | buprenorphine | O | O | O | CO₂H | O | O | 1 | MeSCH₂ |
| A1-475 | buprenorphine | O | O | O | CO₂H | O | O | 1 | MeOCH₂ |
| A1-476 | buprenorphine | O | O | O | CO₂H | S | S | 1 | Ph |
| A1-477 | buprenorphine | O | O | O | CO₂H | S | S | 1 | NMe₂ |
| A1-478 | buprenorphine | O | O | O | CO₂H | S | S | 1 | NBn₂ |
| A1-479 | buprenorphine | O | O | O | CO₂H | O | S | 1 | Ph |
| A1-480 | buprenorphine | O | O | O | CO₂H | O | S | 1 | Me |
| A1-481 | cafiminol | O | O | O | CO₂H | O | O | 1 | methyl |
| A1-482 | cafiminol | O | O | O | CO₂H | O | O | 1 | C(CH₃)₃ |
| A1-483 | cafiminol | O | O | O | CO₂H | O | O | 1 | Ph |
| A1-484 | cafiminol | O | O | O | CO₂H | O | O | 1 | 4-Me-Ph |
| A1-485 | cafiminol | O | O | O | CO₂H | O | O | 1 | 2-pyridyl |
| A1-486 | cafiminol | O | O | O | CO₂H | O | O | 1 | 4-pyridyl |
| A1-487 | cafiminol | O | O | O | CO₂H | O | O | 1 | 2-furyl |
| A1-488 | cafiminol | O | O | O | CO₂H | O | O | 1 | 2-thienyl |
| A1-489 | cafiminol | O | O | O | CO₂H | — | — | 0 | Cl |
| A1-490 | cafiminol | O | O | O | CO₂H | — | — | 0 | {N(Et)₃}⁺Cl⁻ |
| A1-491 | cafiminol | O | O | O | CO₂H | O | O | 1 | CF₃CH₂ |
| A1-492 | cafiminol | O | O | O | CO₂H | O | O | 1 | cyclopropyl |
| A1-493 | cafiminol | O | O | O | CO₂H | O | O | 1 | 2-OMe-Ph |
| A1-494 | cafiminol | O | O | O | CO₂H | O | O | 1 | MeSCH₂ |
| A1-495 | cafiminol | O | O | O | CO₂H | O | O | 1 | MeOCH₂ |
| A1-496 | cafiminol | O | O | O | CO₂H | S | S | 1 | Ph |
| A1-497 | cafiminol | O | O | O | CO₂H | S | S | 1 | NMe₂ |
| A1-498 | cafiminol | O | O | O | CO₂H | S | S | 1 | NBn₂ |
| A1-499 | cafiminol | O | O | O | CO₂H | O | S | 1 | Ph |
| A1-500 | cafiminol | O | O | O | CO₂H | O | S | 1 | Me |
| A1-501 | pentazocine | O | O | O | CO₂H | O | O | 1 | methyl |
| A1-502 | pentazocine | O | O | O | CO₂H | O | O | 1 | C(CH₃)₃ |
| A1-503 | pentazocine | O | O | O | CO₂H | O | O | 1 | Ph |
| A1-504 | pentazocine | O | O | O | CO₂H | O | O | 1 | 4-Me-Ph |
| A1-505 | pentazocine | O | O | O | CO₂H | O | O | 1 | 2-pyridyl |
| A1-506 | pentazocine | O | O | O | CO₂H | O | O | 1 | 4-pyridyl |
| A1-507 | pentazocine | O | O | O | CO₂H | O | O | 1 | 2-furyl |
| A1-508 | pentazocine | O | O | O | CO₂H | O | O | 1 | 2-thienyl |
| A1-509 | pentazocine | O | O | O | CO₂H | — | — | 0 | Cl |
| A1-510 | pentazocine | O | O | O | CO₂H | — | — | 0 | {N(Et)₃}⁺Cl⁻ |
| A1-511 | pentazocine | O | O | O | CO₂H | O | O | 1 | CF₃CH₂ |
| A1-512 | pentazocine | O | O | O | CO₂H | O | O | 1 | cyclopropyl |
| A1-513 | pentazocine | O | O | O | CO₂H | O | O | 1 | 2-OMe-Ph |
| A1-514 | pentazocine | O | O | O | CO₂H | O | O | 1 | MeSCH₂ |
| A1-515 | pentazocine | O | O | O | CO₂H | O | O | 1 | MeOCH₂ |
| A1-516 | pentazocine | O | O | O | CO₂H | S | S | 1 | Ph |
| A1-517 | pentazocine | O | O | O | CO₂H | S | S | 1 | NMe₂ |
| A1-518 | pentazocine | O | O | O | CO₂H | S | S | 1 | NBn₂ |
| A1-519 | pentazocine | O | O | O | CO₂H | O | S | 1 | Ph |
| A1-520 | pentazocme | O | O | O | CO₂H | O | S | 1 | Me |
| A1-521 | scopolamine | O | O | O | CO₂H | O | O | 1 | methyl |
| A1-522 | scopolamine | O | O | O | CO₂H | O | O | 1 | C(CH₃)₃ |
| A1-523 | scopolamine | O | O | O | CO₂H | O | O | 1 | Ph |
| A1-524 | scopolamine | O | O | O | CO₂H | O | O | 1 | 4-Me-Ph |
| A1-525 | scopolamine | O | O | O | CO₂H | O | O | 1 | 2-pyridyl |
| A1-526 | scopolamine | O | O | O | CO₂H | O | O | 1 | 4-pyridyl |
| A1-527 | scopolamine | O | O | O | CO₂H | O | O | 1 | 2-furyl |
| A1-528 | scopolamine | O | O | O | CO₂H | O | O | 1 | 2-thienyl |
| A1-529 | scopolamine | O | O | O | CO₂H | — | — | 0 | Cl |
| A1-530 | scopolamine | O | O | O | CO₂H | — | — | 0 | {N(Et)₃}⁺Cl⁻ |
| A1-531 | scopolamine | O | O | O | CO₂H | O | O | 1 | CF₃CH₂ |
| A1-532 | scopolamine | O | O | O | CO₂H | O | O | 1 | cyclopropyl |
| A1-533 | scopolamine | O | O | O | CO₂H | O | O | 1 | 2-OMe-Ph |
| A1-534 | scopolamine | O | O | O | CO₂H | O | O | 1 | MeSCH₂ |
| A1-535 | scopolamine | O | O | O | CO₂H | O | O | 1 | MeOCH₂ |
| A1-536 | scopolamine | O | O | O | CO₂H | S | S | 1 | Ph |
| A1-537 | scopolamine | O | O | O | CO₂H | S | S | 1 | NMe₂ |
| A1-538 | scopolamine | O | O | O | CO₂H | S | S | 1 | NBn₂ |
| A1-539 | scopolamine | O | O | O | CO₂H | O | S | 1 | Ph |
| A1-540 | scopolamine | O | O | O | CO₂H | O | S | 1 | Me |
| A1-541 | tramadol | O | O | O | CO₂H | O | O | 1 | methyl |
| A1-542 | tramadol | O | O | O | CO₂H | O | O | 1 | C(CH₃)₃ |
| A1-543 | tramadol | O | O | O | CO₂H | O | O | 1 | Ph |
| A1-544 | tramadol | O | O | O | CO₂H | O | O | 1 | 4-Me-Ph |
| A1-545 | tramadol | O | O | O | CO₂H | O | O | 1 | 2-pyridyl |
| A1-546 | tramadol | O | O | O | CO₂H | O | O | 1 | 4-pyridyl |
| A1-547 | tramadol | O | O | O | CO₂H | O | O | 1 | 2-furyl |
| A1-548 | tramadol | O | O | O | CO₂H | O | O | 1 | 2-thienyl |
| A1-549 | tramadol | O | O | O | CO₂H | — | — | 0 | Cl |
| A1-550 | tramadol | O | O | O | CO₂H | — | — | 0 | {N(Et)₃}⁺Cl⁻ |
| A1-551 | tramadol | O | O | O | CO₂H | O | O | 1 | CF₃CH₂ |
| A1-552 | tramadol | O | O | O | CO₂H | O | O | 1 | cyclopropyl |
| A1-553 | tramadol | O | O | O | CO₂H | O | O | 1 | 2-OMe-Ph |
| A1-554 | tramadol | O | O | O | CO₂H | O | O | 1 | MeSCH₂ |
| A1-555 | tramadol | O | O | O | CO₂H | O | O | 1 | MeOCH₂ |
| A1-556 | tramadol | O | O | O | CO₂H | S | S | 1 | Ph |
| A1-557 | tramadol | O | O | O | CO₂H | S | S | 1 | NMe₂ |
| A1-558 | tramadol | O | O | O | CO₂H | S | S | 1 | NBn₂ |
| A1-559 | tramadol | O | O | O | CO₂H | O | S | 1 | Ph |
| A1-560 | tramadol | O | O | O | CO₂H | O | S | 1 | Me |
| A1-561 | quinidine | O | O | O | CO₂H | O | O | 1 | methyl |
| A1-562 | quinidine | O | O | O | CO₂H | O | O | 1 | C(CH₃)₃ |
| A1-563 | quinidine | O | O | O | CO₂H | O | O | 1 | Ph |
| A1-564 | quinidine | O | O | O | CO₂H | O | O | 1 | 4-Me-Ph |
| A1-565 | quinidine | O | O | O | CO₂H | O | O | 1 | 2-pyridyl |
| A1-566 | quinidine | O | O | O | CO₂H | O | O | 1 | 4-pyridyl |
| A1-567 | quinidine | O | O | O | CO₂H | O | O | 1 | 2-furyl |
| A1-568 | quinidine | O | O | O | CO₂H | O | O | 1 | 2-thienyl |
| A1-569 | quinidine | O | O | O | CO₂H | — | — | 0 | Cl |
| A1-570 | quinidine | O | O | O | CO₂H | — | — | 0 | {N(Et)₃}⁺Cl⁻ |
| A1-571 | quinidine | O | O | O | CO₂H | O | O | 1 | CF₃CH₂ |
| A1-572 | quinidine | O | O | O | CO₂H | O | O | 1 | cyclopropyl |
| A1-573 | quinidine | O | O | O | CO₂H | O | O | 1 | 2-OMe-Ph |
| A1-574 | quinidine | O | O | O | CO₂H | O | O | 1 | MeSCH₂ |
| A1-575 | quinidine | O | O | O | CO₂H | O | O | 1 | MeOCH₂ |
| A1-576 | quinidine | O | O | O | CO₂H | S | S | 1 | Ph |
| A1-577 | quinidine | O | O | O | CO₂H | S | S | 1 | NMe₂ |
| A1-578 | quinidine | O | O | O | CO₂H | S | S | 1 | NBn₂ |
| A1-579 | quinidine | O | O | O | CO₂H | O | S | 1 | Ph |
| A1-580 | quinidine | O | O | O | CO₂H | O | S | 1 | Me |
| A1-581 | methimazole | S | O | O | CO₂H | O | O | 1 | methyl |
| A1-582 | methiinazole | S | O | O | CO₂H | O | O | 1 | C(CH₃)₃ |
| A1-583 | methimazole | S | O | O | CO₂H | O | O | 1 | Ph |
| A1-584 | methimazole | S | O | O | CO₂H | O | O | 1 | 4-Me-Ph |
| A1-585 | methimazole | S | O | O | CO₂H | O | O | 1 | 2-pyridyl |
| A1-586 | methimazole | S | O | O | CO₂H | O | O | 1 | 4-pyridyl |
| A1-587 | methimazole | S | O | O | CO₂H | O | O | 1 | 2-furyl |
| A1-588 | methimazole | S | O | O | CO₂H | O | O | 1 | 2-thienyl |
| A1-589 | methimazole | S | O | O | CO₂H | — | — | 0 | Cl |
| A1-590 | methimazole | S | O | O | CO₂H | — | — | 0 | {N(Et)₃}⁺Cl⁻ |
| A1-591 | methimazole | S | O | O | CO₂H | O | O | 1 | CF₃CH₂ |
| A1-592 | methimazole | S | O | O | CO₂H | O | O | 1 | cyclopropyl |
| A1-593 | methimazole | S | O | O | CO₂H | O | O | 1 | 2-OMe-Ph |
| A1-594 | methimazole | S | O | O | CO₂H | O | O | 1 | MeSCH₂ |
| A1-595 | methimazole | S | O | O | CO₂H | O | O | 1 | MeOCH₂ |
| A1-596 | methimazole | S | O | O | CO₂H | S | S | 1 | Ph |
| A1-597 | methimazole | S | O | O | CO₂H | S | S | 1 | NMe₂ |
| A1-598 | methimazole | S | O | O | CO₂H | S | S | 1 | NBn₂ |
| A1-599 | methimazole | S | O | O | CO₂H | O | S | 1 | Ph |
| A1-600 | methimazole | S | O | O | CO₂H | O | S | 1 | Me |
| A1-601 | haloperidol | O | O | O | CO₂H | O | O | 1 | methyl |
| A1-602 | haloperidol | O | O | O | CO₂H | O | O | 1 | C(CH₃)₃ |
| A1-603 | haloperidol | O | O | O | CO₂H | O | O | 1 | Ph |
| A1-604 | haloperidol | O | O | O | CO₂H | O | O | 1 | 4-Me-Ph |
| A1-605 | haloperidol | O | O | O | CO₂H | O | O | 1 | 2-pyridyl |
| A1-606 | haloperidol | O | O | O | CO₂H | O | O | 1 | 4-pyridyl |
| A1-607 | haloperidol | O | O | O | CO₂H | O | O | 1 | 2-furyl |
| A1-608 | haloperidol | O | O | O | CO₂H | O | O | 1 | 2-thienyl |
| A1-609 | haloperidol | O | O | O | CO₂H | — | — | 0 | Cl |
| A1-610 | haloperidol | O | O | O | CO₂H | — | — | 0 | {N(Et)₃}⁺Cl⁻ |
| A1-611 | haloperidol | O | O | O | CO₂H | O | O | 1 | CF₃CH₂ |
| A1-612 | haloperidol | O | O | O | CO₂H | O | O | 1 | cyclopropyl |
| A1-613 | haloperidol | O | O | O | CO₂H | O | O | 1 | 2-OMe-Ph |
| A1-614 | haloperidol | O | O | O | CO₂H | O | O | 1 | MeSCH₂ |
| A1-615 | haloperidol | O | O | O | CO₂H | O | O | 1 | MeOCH₂ |
| A1-616 | haloperidol | O | O | O | CO₂H | S | S | 1 | Ph |
| A1-617 | haloperidol | O | O | O | CO₂H | S | S | 1 | NMe₂ |
| A1-618 | haloperidol | O | O | O | CO₂H | S | S | 1 | NBn₂ |
| A1-619 | haloperidol | O | O | O | CO₂H | O | S | 1 | Ph |
| A1-620 | haloperidol | O | O | O | CO₂H | O | S | 1 | Me |
| A1-621 | hydroxyzine | O | O | O | CO₂H | O | O | 1 | methyl |
| A1-622 | hydroxyzine | O | O | O | CO₂H | O | O | 1 | C(CH₃)₃ |
| A1-623 | hydroxyzine | O | O | O | CO₂H | O | O | 1 | Ph |
| A1-624 | hydroxyzine | O | O | O | CO₂H | O | O | 1 | 4-Me-Ph |
| A1-625 | hydroxyzine | O | O | O | CO₂H | O | O | 1 | 2-pyridyl |
| A1-626 | hydroxyzine | O | O | O | CO₂H | O | O | 1 | 4-pyridyl |
| A1-627 | hydroxyzine | O | O | O | CO₂H | O | O | 1 | 2-furyl |
| A1-628 | hydroxyzine | O | O | O | CO₂H | O | O | 1 | 2-thienyl |
| A1-629 | hydroxyzine | O | O | O | CO₂H | — | — | 0 | Cl |
| A1-630 | hydroxyzine | O | O | O | CO₂H | — | — | 0 | {N(Et)₃}⁺Cl⁻ |
| A1-631 | hydroxyzine | O | O | O | CO₂H | O | O | 1 | CF₃CH₂ |
| A1-632 | hydroxyzine | O | O | O | CO₂H | O | O | 1 | cyclopropyl |
| A1-633 | hydroxyzine | O | O | O | CO₂H | O | O | 1 | 2-OMe-Ph |
| A1-634 | hydroxyzine | O | O | O | CO₂H | O | O | 1 | MeSCH₂ |
| A1-635 | hydroxyzine | O | O | O | CO₂H | O | O | 1 | MeOCH₂ |
| A1-636 | hydroxyzine | O | O | O | CO₂H | S | S | 1 | Ph |
| A1-637 | hydroxyzine | O | O | O | CO₂H | S | S | 1 | NMe₂ |
| A1-638 | hydroxyzine | O | O | O | CO₂H | S | S | 1 | NBn₂ |
| A1-639 | hydroxyzine | O | O | O | CO₂H | O | S | 1 | Ph |
| A1-640 | hydroxyzine | O | O | O | CO₂H | O | S | 1 | Me |
| A1-641 | lorazepam | O | O | O | CO₂H | O | O | 1 | methyl |
| A1-642 | lorazepam | O | O | O | CO₂H | O | O | 1 | C(CH₃)₃ |
| A1-643 | lorazepam | O | O | O | CO₂H | O | O | 1 | Ph |
| A1-644 | lorazepam | O | O | O | CO₂H | O | O | 1 | 4-Me-Ph |
| A1-645 | lorazepam | O | O | O | CO₂H | O | O | 1 | 2-pyridyl |
| A1-646 | lorazepam | O | O | O | CO₂H | O | O | 1 | 4-pyridyl |
| A1-647 | lorazepam | O | O | O | CO₂H | O | O | 1 | 2-furyl |
| A1-648 | lorazepam | O | O | O | CO₂H | O | O | 1 | 2-thienyl |
| A1-649 | lorazepam | O | O | O | CO₂H | — | — | 0 | Cl |
| A1-650 | lorazepam | O | O | O | CO₂H | — | — | 0 | {N(Et)₃}⁺Cl⁻ |
| A1-651 | lorazepam | O | O | O | CO₂H | O | O | 1 | CF₃CH₂ |
| A1-652 | lorazepam | O | O | O | CO₂H | O | O | 1 | cyclopropyl |
| A1-653 | lorazepam | O | O | O | CO₂H | O | O | 1 | 2-OMe-Ph |
| A1-654 | lorazepam | O | O | O | CO₂H | O | O | 1 | MeSCH₂ |
| A1-655 | lorazepam | O | O | O | CO₂H | O | O | 1 | MeOCH₂ |
| A1-656 | lorazepam | O | O | O | CO₂H | S | S | 1 | Ph |
| A1-657 | lorazepam | O | O | O | CO₂H | S | S | 1 | NMe₂ |
| A1-658 | lorazepam | O | O | O | CO₂H | S | S | 1 | NBn₂ |
| A1-659 | lorazepam | O | O | O | CO₂H | O | S | 1 | Ph |
| A1-660 | lorazepam | O | O | O | CO₂H | O | S | 1 | Me |
| A1-661 | iodoquinol | O | O | O | CO₂H | O | O | 1 | methyl |
| A1-662 | iodoquinol | O | O | O | CO₂H | O | O | 1 | C(CH₃)₃ |
| A1-663 | iodoquinol | O | O | O | CO₂H | O | O | 1 | Ph |
| A1-664 | iodoquinol | O | O | O | CO₂H | O | O | 1 | 4-Me-Ph |
| A1-665 | iodoquinol | O | O | O | CO₂H | O | O | 1 | 2-pyridyl |
| A1-666 | iodoquinol | O | O | O | CO₂H | O | O | 1 | 4-pyridyl |
| A1-667 | iodoquinol | O | O | O | CO₂H | O | O | 1 | 2-furyl |
| A1-668 | iodoquinol | O | O | O | CO₂H | O | O | 1 | 2-thienyl |
| A1-669 | iodoquinol | O | O | O | CO₂H | — | — | 0 | Cl |
| A1-670 | iodoquinol | O | O | O | CO₂H | — | — | 0 | {N(Et)₃}⁺Cl⁻ |
| A1-671 | iodoquinol | O | O | O | CO₂H | O | O | 1 | CF₃CH₂ |
| A1-672 | iodoquinol | O | O | O | CO₂H | O | O | 1 | cyclopropyl |
| A1-673 | iodoquinol | O | O | O | CO₂H | O | O | 1 | 2-OMe-Ph |
| A1-674 | iodoquinol | O | O | O | CO₂H | O | O | 1 | MeSCH₂ |
| A1-675 | iodoquinol | O | O | O | CO₂H | O | O | 1 | MeOCH₂ |
| A1-676 | iodoquinol | O | O | O | CO₂H | S | S | 1 | Ph |
| A1-677 | iodoquinol | O | O | O | CO₂H | S | S | 1 | NMe₂ |
| A1-678 | iodoquinol | O | O | O | CO₂H | S | S | 1 | NBn₂ |
| A1-679 | iodoquinol | O | O | O | CO₂H | O | S | 1 | Ph |
| A1-680 | iodoquinol | O | O | O | CO₂H | O | S | 1 | Me |
| A1-681 | perphenazine | O | O | O | CO₂H | O | O | 1 | methyl |
| A1-682 | perphenazine | O | O | O | CO₂H | O | O | 1 | C(CH₃)₃ |
| A1-683 | perphenazine | O | O | O | CO₂H | O | O | 1 | Ph |
| A1-684 | perphenazine | O | O | O | CO₂H | O | O | 1 | 4-Me-Ph |
| A1-685 | perphenazine | O | O | O | CO₂H | O | O | 1 | 2-pyridyl |
| A1-686 | perphenazine | O | O | O | CO₂H | O | O | 1 | 4-pyridyl |
| A1-687 | perphenazine | O | O | O | CO₂H | O | O | 1 | 2-furyl |
| A1-688 | perphenazine | O | O | O | CO₂H | O | O | 1 | 2-thienyl |
| A1-689 | perphenazine | O | O | O | CO₂H | — | — | 0 | Cl |
| A1-690 | perphenazine | O | O | O | CO₂H | — | — | 0 | {N(Et)₃}⁺Cl⁻ |
| A1-691 | perphenazine | O | O | O | CO₂H | O | O | 1 | CF₃CH₂ |
| A1-692 | perphenazine | O | O | O | CO₂H | O | O | 1 | cyclopropyl |
| A1-693 | perphenazine | O | O | O | CO₂H | O | O | 1 | 2-OMe-Ph |
| A1-694 | perphenazine | O | O | O | CO₂H | O | O | 1 | MeSCH₂ |
| A1-695 | perphenazine | O | O | O | CO₂H | O | O | 1 | MeOCH₂ |
| A1-696 | perphenazine | O | O | O | CO₂H | S | S | 1 | Ph |
| A1-697 | perphenazine | O | O | O | CO₂H | S | S | 1 | NMe₂ |
| A1-698 | perphenazine | O | O | O | CO₂H | S | S | 1 | NBn₂ |
| A1-699 | perphenazine | O | O | O | CO₂H | O | S | 1 | Ph |
| A1-700 | perphenazine | O | O | O | CO₂H | O | S | 1 | Me |
| A1-701 | mazindol | O | O | O | CO₂H | O | O | 1 | methyl |
| A1-702 | mazindol | O | O | O | CO₂H | O | O | 1 | C(CH₃)₃ |
| A1-703 | mazindol | O | O | O | CO₂H | O | O | 1 | Ph |
| A1-704 | mazindol | O | O | O | CO₂H | O | O | 1 | 4-Me-Ph |
| A1-705 | mazindol | O | O | O | CO₂H | O | O | 1 | 2-pyridyl |
| A1-706 | mazindol | O | O | O | CO₂H | O | O | 1 | 4-pyridyl |
| A1-707 | mazindol | O | O | O | CO₂H | O | O | 1 | 2-furyl |
| A1-708 | mazindol | O | O | O | CO₂H | O | O | 1 | 2-thienyl |
| A1-709 | mazindol | O | O | O | CO₂H | — | — | O | Cl |
| A1-710 | mazindol | O | O | O | CO₂H | — | — | 0 | {N(Et)₃}⁺Cl⁻ |
| A1-711 | mazindol | O | O | O | CO₂H | O | O | 1 | CF₃CH₂ |
| A1-712 | mazindol | O | O | O | CO₂H | O | O | 1 | cyclopropyl |
| A1-713 | mazindol | O | O | O | CO₂H | O | O | 1 | 2-OMe-Ph |
| A1-714 | mazindol | O | O | O | CO₂H | O | O | 1 | MeSCH₂ |
| A1-715 | mazindol | O | O | O | CO₂H | O | O | 1 | MeOCH₂ |
| A1-716 | mazindol | O | O | O | CO₂H | S | S | 1 | Ph |
| A1-717 | mazindol | O | O | O | CO₂H | S | S | 1 | NMe₂ |
| A1-718 | mazindol | O | O | O | CO₂H | S | S | 1 | NBn₂ |
| A1-719 | mazindol | O | O | O | CO₂H | O | S | 1 | Ph |
| A1-720 | mazmdol | O | O | O | CO₂H | O | S | 1 | Me |
| A1-721 | astemizole | N | O | O | CO₂H | O | O | 1 | methyl |
| A1-722 | astemizole | N | O | O | CO₂H | O | O | 1 | C(CH₃)₃ |
| A1-723 | astemizole | N | O | O | CO₂H | O | O | 1 | Ph |
| A1-724 | astemizole | N | O | O | CO₂H | O | O | 1 | 4-Me-Ph |
| A1-725 | astemizole | N | O | O | CO₂H | O | O | 1 | 2-pyridyl |
| A1-726 | astemizole | N | O | O | CO₂H | O | O | 1 | 4-pyridyl |
| A1-727 | astemizole | N | O | O | CO₂H | O | O | 1 | 2-furyl |
| A1-728 | astemizole | N | O | O | CO₂H | O | O | 1 | 2-thienyl |
| A1-729 | astemizole | N | O | O | CO₂H | — | — | 0 | Cl |
| A1-730 | astemizole | N | O | O | CO₂H | — | — | 0 | {N(Et)₃}⁺Cl⁻ |
| A1-731 | astemizole | N | O | O | CO₂H | O | O | 1 | CF₃CH₂ |
| A1-732 | astemizole | N | O | O | CO₂H | O | O | 1 | cyclopropyl |
| A1-733 | astemizole | N | O | O | CO₂H | O | O | 1 | 2-OMe-Ph |
| A1-734 | astemizole | N | O | O | CO₂H | O | O | 1 | MeSCH₂ |
| A1-735 | astemizole | N | O | O | CO₂H | O | O | 1 | MeOCH₂ |
| A1-736 | astemizole | N | O | O | CO₂H | S | S | 1 | Ph |
| A1-737 | astemizole | N | O | O | CO₂H | S | S | 1 | NMe₂ |
| A1-738 | astemizole | N | O | O | CO₂H | S | S | 1 | NBn₂ |
| A1-739 | astemizole | N | O | O | CO₂H | O | S | 1 | Ph |
| A1-740 | astemizole | N | O | O | CO₂H | O | S | 1 | Me |
| A1-741 | amoxapine | N | O | O | CO₂H | O | O | 1 | methyl |
| A1-742 | amoxapine | N | O | O | CO₂H | O | O | 1 | C(CH₃)₃ |
| A1-743 | amoxapine | N | O | O | CO₂H | O | O | 1 | Ph |
| A1-744 | amoxapine | N | O | O | CO₂H | O | O | 1 | 4-Me-Ph |
| A1-745 | amoxapine | N | O | O | CO₂H | O | O | 1 | 2-pyridyl |
| A1-746 | amoxapine | N | O | O | CO₂H | O | O | 1 | 4-pyridyl |
| A1-747 | amoxapine | N | O | O | CO₂H | O | O | 1 | 2-furyl |
| A1-748 | amoxapine | N | O | O | CO₂H | O | O | 1 | 2-thienyl |
| A1-749 | amoxapine | N | O | O | CO₂H | — | — | 0 | Cl |
| A1-750 | amoxapine | N | O | O | CO₂H | — | — | 0 | {N(Et)₃}⁺Cl⁻ |
| A1-751 | amoxapine | N | O | O | CO₂H | O | O | 1 | CF₃CH₂ |
| A1-752 | amoxapine | N | O | O | CO₂H | O | O | 1 | cyclopropyl |
| A1-753 | amoxapine | N | O | O | CO₂H | O | O | 1 | 2-OMe-Ph |
| A1-754 | amoxapine | N | O | O | CO₂H | O | O | 1 | MeSCH₂ |
| A1-755 | amoxapine | N | O | O | CO₂H | O | O | 1 | MeOCH₂ |
| A1-756 | amoxapine | N | O | O | CO₂H | S | S | 1 | Ph |
| A1-757 | amoxapine | N | O | O | CO₂H | S | S | 1 | NMe₂ |
| A1-758 | amoxapine | N | O | O | CO₂H | S | S | 1 | NBn₂ |
| A1-759 | amoxapine | N | O | O | CO₂H | O | S | 1 | Ph |
| A1-760 | amoxapine | N | O | O | CO₂H | O | S | 1 | Me |
| A1-761 | ciprofloxacin | N | O | O | CO₂H | O | O | 1 | methyl |
| A1-762 | ciprofloxacin | N | O | O | CO₂H | O | O | 1 | C(CH₃)₃ |
| A1-763 | ciprofloxacin | N | O | O | CO₂H | O | O | 1 | Ph |
| A1-764 | ciprofloxacin | N | O | O | CO₂H | O | O | 1 | 4-Me-Ph |
| A1-765 | ciprofloxacin | N | O | O | CO₂H | O | O | 1 | 2-pyridyl |
| A1-766 | ciprofloxacin | N | O | O | CO₂H | O | O | 1 | 4-pyridyl |
| A1-767 | ciprofloxacin | N | O | O | CO₂H | O | O | 1 | 2-furyl |
| A1-768 | ciprofloxacin | N | O | O | CO₂H | O | O | 1 | 2-thienyl |
| A1-769 | ciprofloxacin | N | O | O | CO₂H | — | — | 0 | Cl |
| A1-770 | ciprofloxacin | N | O | O | CO₂H | — | — | 0 | {N(Et)₃}⁺Cl⁻ |
| A1-771 | ciprofloxacin | N | O | O | CO₂H | O | O | 1 | CF₃CH₂ |
| A1-772 | ciprofloxacin | N | O | O | CO₂H | O | O | 1 | cyclopropyl |
| A1-773 | ciprofloxacin | N | O | O | CO₂H | O | O | 1 | 2-OMe-Ph |
| A1-774 | ciprofloxacin | N | O | O | CO₂H | O | O | 1 | MeSCH₂ |
| A1-775 | ciprofloxacin | N | O | O | CO₂H | O | O | 1 | MeOCH₂ |
| A1-776 | ciprofloxacin | N | O | O | CO₂H | S | S | 1 | Ph |
| A1-777 | ciprofloxacin | N | O | O | CO₂H | S | S | 1 | NMe₂ |
| A1-778 | ciprofloxacin | N | O | O | CO₂H | S | S | 1 | NBn₂ |
| A1-779 | ciprofloxacin | N | O | O | CO₂H | O | S | 1 | Ph |
| A1-780 | ciprofloxacin | N | O | O | CO₂H | O | S | 1 | Me |
| A1-781 | chloroquine | N | O | O | CO₂H | O | O | 1 | methyl |
| A1-782 | chloroquine | N | O | O | CO₂H | O | O | 1 | C(CH₃)₃ |
| A1-783 | chloroquine | N | O | O | CO₂H | O | O | 1 | Ph |
| A1-784 | chloroquine | N | O | O | CO₂H | O | O | 1 | 4-Me-Ph |
| A1-785 | chloroquine | N | O | O | CO₂H | O | O | 1 | 2-pyridyl |
| A1-786 | chloroquine | N | O | O | CO₂H | O | O | 1 | 4-pyridyl |
| A1-787 | chloroquine | N | O | O | CO₂H | O | O | 1 | 2-furyl |
| A1-788 | chloroquine | N | O | O | CO₂H | O | O | 1 | 2-thienyl |
| A1-789 | chloroquine | N | O | O | CO₂H | — | — | 0 | Cl |
| A1-790 | chloroquine | N | O | O | CO₂H | — | — | 0 | {N(Et)₃}⁺Cl⁻ |
| A1-791 | chloroquine | N | O | O | CO₂H | O | O | 1 | CF₃CH₂ |
| A1-792 | chloroquine | N | O | O | CO₂H | O | O | 1 | cyclopropyl |
| A1-793 | chloroquine | N | O | O | CO₂H | O | O | 1 | 2-OMe-Ph |
| AI-794 | chloroquine | N | O | O | CO₂H | O | O | 1 | MeSCH₂ |
| A1-795 | chloroquine | N | O | O | CO₂H | O | O | 1 | MeOCH₂ |
| A1-796 | chloroquine | N | O | O | CO₂H | S | S | 1 | Ph |
| A1-797 | chloroquine | N | O | O | CO₂H | S | S | 1 | NMe₂ |
| A1-798 | chloroquine | N | O | O | CO₂H | S | S | 1 | NBn₂ |
| A1-799 | chloroquine | N | O | O | CO₂H | O | S | 1 | Ph |
| A1-800 | chloroquine | N | O | O | CO₂H | O | S | 1 | Me |
| A1-801 | chlordiazepoxide | N | O | O | CO₂H | O | O | 1 | methyl |
| A1-802 | chlordiazepoxide | N | O | O | CO₂H | O | O | 1 | C(CH₃)₃ |
| A1-503 | chlordiazepoxide | N | O | O | CO₂H | O | O | 1 | Ph |
| A1-804 | chlordiazepoxide | N | O | O | CO₂H | O | O | 1 | 4-Me-Ph |
| A1-805 | chlordiazepoxide | N | O | O | CO₂H | O | O | 1 | 2-pyridyl |
| A1-806 | chlordiazepoxide | N | O | O | CO₂H | O | O | 1 | 4-pyridyl |
| A1-807 | chlordiazepoxide | N | O | O | CO₂H | O | O | 1 | 2-furyl |
| A1-808 | chlordiazepoxide | N | O | O | CO₂H | O | O | 1 | 2-thienyl |
| A1-809 | chlordiazepoxide | N | O | O | CO₂H | — | — | 0 | Cl |
| A1-810 | chlordiazepoxide | N | O | O | CO₂H | — | — | 0 | {N(Et)₃}⁺Cl⁻ |
| A1-811 | chlordiazepoxide | N | O | O | CO₂H | O | O | 1 | CF₃CH₂ |
| A1-812 | chlordiazepoxide | N | O | O | CO₂H | O | O | 1 | cyclopropyl |
| A1-813 | chlordiazepoxide | N | O | O | CO₂H | O | O | 1 | 2-OMe-Ph |
| A1-814 | chlordiazepoxide | N | O | O | CO₂H | O | O | 1 | MeSCH₂ |
| A1-815 | chlordiazepoxide | N | O | O | CO₂H | O | O | 1 | MeOCH₂ |
| A1-816 | chlordiazepoxide | N | O | O | CO₂H | S | S | 1 | Ph |
| A1-817 | chlordiazepoxide | N | O | O | CO₂H | S | S | 1 | NMe₂ |
| A1-818 | chlordiazepoxide | N | O | O | CO₂H | S | S | 1 | NBn₂ |
| A1-819 | chlordiazepoxide | N | O | O | CO₂H | O | S | 1 | Ph |
| A1-820 | chlordiazepoxide | N | O | O | CO₂H | O | S | 1 | Me |
| A1-821 | clozapine | N | O | O | CO₂H | O | O | 1 | methyl |
| A1-822 | clozapine | N | O | O | CO₂H | O | O | 1 | C(CH₃)₃ |
| A1-823 | clozapine | N | O | O | CO₂H | O | O | 1 | Ph |
| A1-824 | clozapine | N | O | O | CO₂H | O | O | 1 | 4-Me-Ph |
| A1-825 | clozapine | N | O | O | CO₂H | O | O | 1 | 2-pyridyl |
| A1-826 | clozapine | N | O | O | CO₂H | O | O | 1 | 4-pyridyl |
| A1-827 | clozapine | N | O | O | CO₂H | O | O | 1 | 2-furyl |
| A1-828 | clozapine | N | O | O | CO₂H | O | O | 1 | 2-thienyl |
| A1-829 | clozapine | N | O | O | CO₂H | — | — | 0 | Cl |
| A1-830 | clozapine | N | O | O | CO₂H | — | — | 0 | {N(Et)₃}⁺Cl⁻ |
| A1-831 | clozapine | N | O | O | CO₂H | O | O | 1 | CF₃CH₂ |
| A1-832 | clozapine | N | O | O | CO₂H | O | O | 1 | cyclopropyl |
| A1-833 | clozapine | N | O | O | CO₂H | O | O | 1 | 2-OMe-Ph |
| A1-834 | clozapine | N | O | O | CO₂H | O | O | 1 | MeSCH₂ |
| A1-835 | clozapine | N | O | O | CO₂H | O | O | 1 | MeOCH₂ |
| A1-836 | clozapine | N | O | O | CO₂H | S | S | 1 | Ph |
| A1-837 | clozapine | N | O | O | CO₂H | S | S | 1 | NMe₂ |
| A1-838 | clozapine | N | O | O | CO₂H | S | S | 1 | NBn₂ |
| A1-839 | clozapine | N | O | O | CO₂H | O | S | 1 | Ph |
| A1-840 | clozapine | N | O | O | CO₂H | O | S | 1 | Me |
| A1-841 | desipramine | N | O | O | CO₂H | O | O | 1 | methyl |
| A1-842 | desipramine | N | O | O | CO₂H | O | O | 1 | C(CH₃)₃ |
| A1-843 | desipramine | N | O | O | CO₂H | O | O | 1 | Ph |
| A1-844 | desipramine | N | O | O | CO₂H | O | O | 1 | 4-Me-Ph |
| A1-845 | desipramine | N | O | O | CO₂H | O | O | 1 | 2-pyridyl |
| A1-846 | desipramine | N | O | O | CO₂H | O | O | 1 | 4-pyridyl |
| A1-847 | desipramine | N | O | O | CO₂H | O | O | 1 | 2-furyl |
| A1-848 | desipramine | N | O | O | CO₂H | O | O | 1 | 2-thienyl |
| A1-849 | desipramine | N | O | O | CO₂H | — | — | 0 | Cl |
| A1-850 | desipramine | N | O | O | CO₂H | — | — | 0 | {N(Et)₃}⁺Cl⁻ |
| A1-851 | desipramine | N | O | O | CO₂H | O | O | 1 | CF₃CH₂ |
| A1-852 | desipramine | N | O | O | CO₂H | O | O | 1 | cyclopropyl |
| A1-853 | desipramine | N | O | O | CO₂H | O | O | 1 | 2-OMe-Ph |
| A1-854 | desipramine | N | O | O | CO₂H | O | O | 1 | MeSCH₂ |
| A1-855 | desipramine | N | O | O | CO₂H | O | O | 1 | MeOCH₂ |
| A1-856 | desipramine | N | O | O | CO₂H | S | S | 1 | Ph |
| A1-857 | desipramine | N | O | O | CO₂H | S | S | 1 | NMe₂ |
| A1-858 | desipramine | N | O | O | CO₂H | S | S | 1 | NBn₂ |
| A1-859 | desipramine | N | O | O | CO₂H | O | S | 1 | Ph |
| A1-860 | desipramine | N | O | O | CO₂H | O | S | 1 | Me |
| A1-861 | enoxacin | N | O | O | CO₂H | O | O | 1 | methyl |
| A1-862 | enoxacin | N | O | O | CO₂H | O | O | 1 | C(CH₃)₃ |
| A1-563 | enoxacin | N | O | O | CO₂H | O | O | 1 | Ph |
| A1-864 | enoxacin | N | O | O | CO₂H | O | O | 1 | 4-Me-Ph |
| A1-865 | enoxacin | N | O | O | CO₂H | O | O | 1 | 2-pyridyl |
| A1-866 | enoxacin | N | O | O | CO₂H | O | O | 1 | 4-pyridyl |
| A1-867 | enoxacin | N | O | O | CO₂H | O | O | 1 | 2-furyl |
| A1-868 | enoxacin | N | O | O | CO₂H | O | O | 1 | 2-thienyl |
| A1-869 | enoxacin | N | O | O | CO₂H | — | — | 0 | Cl |
| A1-870 | enoxacin | N | O | O | CO₂H | — | — | 0 | {N(Et)₃}⁺Cl⁻ |
| A1-871 | enoxacin | N | O | O | CO₂H | O | O | 1 | CF₃CH₂ |
| A1-872 | enoxacin | N | O | O | CO₂H | O | O | 1 | cyclopropyl |
| A1-873 | enoxacin | N | O | O | CO₂H | O | O | 1 | 2-OMe-Ph |
| A1-874 | enoxacin | N | O | O | CO₂H | O | O | 1 | MeSCH₂ |
| A1-875 | enoxacin | N | O | O | CO₂H | O | O | 1 | MeOCH₂ |
| A1-876 | enoxacin | N | O | O | CO₂H | S | S | 1 | Ph |
| A1-877 | enoxacin | N | O | O | CO₂H | S | S | 1 | NMe₂ |
| A1-878 | enoxacin | N | O | O | CO₂H | S | S | 1 | NBn₂ |
| A1-879 | enoxacin | N | O | O | CO₂H | O | S | 1 | Ph |
| A1-880 | enoxacin | N | O | O | CO₂H | O | S | 1 | Me |
| A1-881 | lomefloxacin | N | O | O | CO₂H | O | O | 1 | methyl |
| A1-882 | lomefloxacin | N | O | O | CO₂H | O | O | 1 | C(CH₃)₃ |
| A1-883 | lomefloxacin | N | O | O | CO₂H | O | O | 1 | Ph |
| A1-884 | lomefloxacin | N | O | O | CO₂H | O | O | 1 | 4-Me-Ph |
| A1-885 | lomefloxacin | N | O | O | CO₂H | O | O | 1 | 2-pyridyl |
| A1-886 | lomefloxacin | N | O | O | CO₂H | O | O | 1 | 4-pyridyl |
| A1-887 | lomefloxacin | N | O | O | CO₂H | O | O | 1 | 2-furyl |
| A1-888 | lomefloxacin | N | O | O | CO₂H | O | O | 1 | 2-thienyl |
| A1-889 | lomefloxacin | N | O | O | CO₂H | — | — | 0 | Cl |
| A1-890 | lomefloxacin | N | O | O | CO₂H | — | — | 0 | {N(Et)₃}⁺Cl⁻ |
| A1-891 | lomefloxacin | N | O | O | CO₂H | O | O | 1 | CF₃CH₂ |
| A1-892 | lomefloxacin | N | O | O | CO₂H | O | O | 1 | cyclopropyl |
| A1-893 | lomefloxacin | N | O | O | CO₂H | O | O | 1 | 2-OMe-Ph |
| A1-894 | lomefloxacin | N | O | O | CO₂H | O | O | 1 | MeSCH₂ |
| A1-895 | lomefloxacin | N | O | O | CO₂H | O | O | 1 | MeOCH₂ |
| A1-896 | lomefloxacin | N | O | O | CO₂H | S | S | 1 | Ph |
| A1-897 | lomefloxacin | N | O | O | CO₂H | S | S | 1 | NMe₂ |
| A1-898 | lomefloxacin | N | O | O | CO₂H | S | S | 1 | NBn₂ |
| A1-899 | lomefloxacin | N | O | O | CO₂H | O | S | 1 | Ph |
| A1-900 | lomefloxacin | N | O | O | CO₂H | O | S | 1 | Me |
| A1-901 | mepivacaine | N | O | O | CO₂H | O | O | 1 | methyl |
| A1-902 | mepivacaine | N | O | O | CO₂H | O | O | 1 | C(CH₃)₃ |
| A1-903 | mepivacaine | N | O | O | CO₂H | O | O | 1 | Ph |
| A1-904 | mepivacaine | N | O | O | CO₂H | O | O | 1 | 4-Me-Ph |
| A1-905 | mepivacaine | N | O | O | CO₂H | O | O | 1 | 2-pyridyl |
| A1-906 | mepivacaine | N | O | O | CO₂H | O | O | 1 | 4-pyridyl |
| A1-907 | mepivacaine | N | O | O | CO₂H | O | O | 1 | 2-furyl |
| A1-908 | mepivacaine | N | O | O | CO₂H | O | O | 1 | 2-thienyl |
| A1-909 | mepivacaine | N | O | O | CO₂H | — | — | 0 | Cl |
| A1-910 | mepivacaine | N | O | O | CO₂H | — | — | 0 | {N(Et)₃}⁺Cl⁻ |
| A1-911 | mepivacaine | N | O | O | CO₂H | O | O | 1 | CF₃CH₂ |
| A1-912 | mepivacaine | N | O | O | CO₂H | O | O | 1 | cyclopropyl |
| A1-913 | mepivacaine | N | O | O | CO₂H | O | O | 1 | 2-OMe-Ph |
| A1-914 | mepivacaine | N | O | O | CO₂H | O | O | 1 | MeSCH₂ |
| A1-915 | mepivacaine | N | O | O | CO₂H | O | O | 1 | MeOCH₂ |
| A1-916 | mepivacaine | N | O | O | CO₂H | S | S | 1 | Ph |
| A1-917 | mepivacaine | N | O | O | CO₂H | S | S | 1 | NMe₂ |
| A1-918 | mepivacaine | N | O | O | CO₂H | S | S | 1 | NBn₂ |
| A1-919 | mepivacaine | N | O | O | CO₂H | O | S | 1 | Ph |
| A1-920 | mepivacaine | N | O | O | CO₂H | O | S | 1 | Me |
| A1-921 | molindone | N | O | O | CO₂H | O | O | 1 | methyl |
| A1-922 | molindone | N | O | O | CO₂H | O | O | 1 | C(CH₃)₃ |
| A1-923 | molindone | N | O | O | CO₂H | O | O | 1 | Ph |
| A1-924 | molindone | N | O | O | CO₂H | O | O | 1 | 4-Me-Ph |
| A1-925 | molindone | N | O | O | CO₂H | O | O | 1 | 2-pyridyl |
| A1-926 | molindone | N | O | O | CO₂H | O | O | 1 | 4-pyridyl |
| A1-927 | molindone | N | O | O | CO₂H | O | O | 1 | 2-furyl |
| A1-928 | molindone | N | O | O | CO₂H | O | O | 1 | 2-thienyl |
| A1-929 | molindone | N | O | O | CO₂H | — | — | 0 | Cl |
| A1-930 | molindone | N | O | O | CO₂H | — | — | 0 | {N(Et)₃}⁺Cl⁻ |
| A1-931 | molindone | N | O | O | CO₂H | O | O | 1 | CF₃CH₂ |
| A1-932 | molindone | N | O | O | CO₂H | O | O | 1 | cyclopropyl |
| A1-933 | molindone | N | O | O | CO₂H | O | O | 1 | 2-OMe-Ph |
| A1-934 | molindone | N | O | O | CO₂H | O | O | 1 | MeSCH₂ |
| A1-935 | molindone | N | O | O | CO₂H | O | O | 1 | MeOCH₂ |
| A1-936 | molindone | N | O | O | CO₂H | S | S | 1 | Ph |
| A1-937 | molindone | N | O | O | CO₂H | S | S | 1 | NMe₂ |
| A1-938 | molindone | N | O | O | CO₂H | S | S | 1 | NBn₂ |
| A1-939 | molindone | N | O | O | CO₂H | O | S | 1 | Ph |
| A1-940 | molindone | N | O | O | CO₂H | O | S | 1 | Me |
| A1-941 | naphazoline | N | O | O | CO₂H | O | O | 1 | methyl |
| A1-942 | naphazoline | N | O | O | COAl | O | O | 1 | C(CH₃)₃ |
| A1-943 | naphazoline | N | O | O | CO₂H | O | O | 1 | Ph |
| A1-944 | naphazoline | N | O | O | CO₂H | O | O | 1 | 4-Me-Ph |
| A1-945 | naphazoline | N | O | O | CO₂H | O | O | 1 | 2-pyridyl |
| A1-946 | naphazoline | N | O | O | CO₂H | O | O | 1 | 4-pyridyl |
| A1-947 | naphazoline | N | O | O | CO₂H | O | O | 1 | 2-furyl |
| A1-948 | naphazoline | N | O | O | CO₂H | O | O | 1 | 2-thienyl |
| A1-949 | naphazoline | N | O | O | CO₂H | — | — | 0 | Cl |
| A1-950 | naphazoline | N | O | O | CO₂H | — | — | 0 | {N(Et)₃}⁺Cl⁻ |
| A1-951 | naphazoline | N | O | O | CO₂H | O | O | 1 | CF₃CH₂ |
| A1-952 | naphazoline | N | O | O | CO₂H | O | O | 1 | cyclopropyl |
| A1-953 | naphazoline | N | O | O | CO₂H | O | O | 1 | 2-OMe-Ph |
| A1-954 | naphazoline | N | O | O | CO₂H | O | O | 1 | MeSCH₂ |
| A1-955 | naphazoline | N | O | O | CO₂H | O | O | 1 | MeOCH₂ |
| A1-956 | naphazoline | N | O | O | CO₂H | S | S | 1 | Ph |
| A1-957 | naphazoline | N | O | O | CO₂H | S | S | 1 | NMe₂ |
| A1-958 | naphazoline | N | O | O | CO₂H | S | S | 1 | NBn₂ |
| A1-959 | naphazoline | N | O | O | CO₂H | O | S | 1 | Ph |
| A1-960 | naphazoline | N | O | O | CO₂H | O | S | 1 | Me |
| A1-961 | norfloxacin | N | O | O | CO₂H | O | O | 1 | methyl |
| A1-962 | norfloxacin | N | O | O | CO₂H | O | O | 1 | C(CH₃)₃ |
| A1-963 | norfloxacin | N | O | O | CO₂H | O | O | 1 | Ph |
| A1-964 | norfloxacin | N | O | O | CO₂H | O | O | 1 | 4-Me-Ph. |
| A1-965 | norfloxacin | N | O | O | CO₂H | O | O | 1 | 2-pyridyl |
| A1-966 | norfloxacin | N | O | O | CO₂H | O | O | 1 | 4-pyridyl |
| A1-967 | norfloxacin | N | O | O | CO₂H | O | O | 1 | 2-furyl |
| A1-968 | norfloxacin | N | O | O | CO₂H | O | O | 1 | 2-thienyl |
| A1-969 | norfloxacin | N | O | O | CO₂H | — | — | 0 | Cl |
| A1-970 | norfloxacin | N | O | O | CO₂H | — | — | 0 | {N(Et)₃}⁺Cl⁻ |
| A1-971 | norfloxacin | N | O | O | CO₂H | O | O | 1 | CF₃CH₂ |
| A1-972 | norfloxacin | N | O | O | CO₂H | O | O | 1 | cyclopropyl |
| A1-973 | norfloxacin | N | O | O | CO₂H | O | O | 1 | 2-OMe-Ph |
| A1-974 | norfloxacin | N | O | O | CO₂H | O | O | 1 | MeSCH₂ |
| A1-975 | norfloxacin | N | O | O | CO₂H | O | O | 1 | MeOCH₂ |
| A1-976 | norfloxacin | N | O | O | CO₂H | S | S | 1 | Ph |
| A1-977 | norfloxacin | N | O | O | CO₂H | S | S | 1 | NMe₂ |
| A1-978 | norfloxacin | N | O | O | CO₂H | S | S | 1 | NBfl2 |
| A1-979 | norfloxacin | N | O | O | CO₂H | O | S | 1 | Ph |
| A1-980 | norfloxacin | N | O | O | CO₂H | O | S | 1 | Me |
| A1-981 | pimozide | N | O | O | CO₂H | O | O | 1 | methyl |
| A1-982 | pimozide | N | O | O | CO₂H | O | O | 1 | O(CH₃)₃ |
| A1-983 | pimozide | N | O | O | CO₂H | O | O | 1 | Ph |
| A1-984 | pimozide | N | O | O | CO₂H | O | O | 1 | 4-Me-Ph |
| A1-985 | pimozide | N | O | O | CO₂H | O | O | 1 | 2-pyridyl |
| A1-986 | pimozide | N | O | O | CO₂H | O | O | 1 | 4-pyridyl |
| A1-987 | pimozide | N | O | O | CO₂H | O | O | 1 | 2-furyl |
| A1-988 | pimozide | N | O | O | CO₂H | O | O | 1 | 2-thienyl |
| A1-989 | pimozide | N | O | O | CO₂H | — | — | 0 | Cl |
| A1-990 | pimozide | N | O | O | CO₂H | — | — | 0 | {N(Et)₃}⁺Cl⁻ |
| A1-991 | pimozide | N | O | O | CO₂H | O | O | 1 | CF₃CH₂ |
| A1-992 | pimozide | N | O | O | CO₂H | O | O | 1 | cyclopropyl |
| A1-993 | pimozide | N | O | O | CO₂H | O | O | 1 | 2-OMe-Ph |
| A1-994 | pimozide | N | O | O | CO₂H | O | O | 1 | MeSCH₂ |
| A1-995 | pimozide | N | O | O | CO₂H | O | O | 1 | MeOCH₂ |
| A1-996 | pimozide | N | O | O | CO₂H | S | S | 1 | Ph |
| A1-997 | pimozide | N | O | O | CO₂H | S | S | 1 | NMe₂ |
| A1-998 | pimozide | N | O | O | CO₂H | S | S | 1 | NBn₂ |
| A1-999 | pimozide | N | O | O | CO₂H | O | S | 1 | Ph |
| A1-1000 | pimozide | N | O | O | CO₂H | O | S | 1 | Me |
| A1-1001 | prazosin | N | O | O | CO₂H | O | O | 1 | methyl |
| A1-1002 | prazosin | N | O | O | CO₂H | O | O | 1 | C(CH₃)₃ |
| A1-1003 | prazosin | N | O | O | CO₂H | O | O | 1 | Ph |
| A1-1004 | prazosin | N | O | O | CO₂H | O | O | 1 | 4-Me-Ph |
| A1-1005 | prazosin | N | O | O | CO₂H | O | O | 1 | 2-pyridyl |
| A1-1006 | prazosin | N | O | O | CO₂H | O | O | 1 | 4-pyridyl |
| A1-1007 | prazosin | N | O | O | CO₂H | O | O | 1 | 2-furyl |
| A1-1008 | prazosin | N | O | O | CO₂H | O | O | 1 | 2-thienyl |
| A1-1009 | prazosin | N | O | O | CO₂H | — | — | 0 | Cl |
| A1-1010 | prazosin | N | O | O | CO₂H | — | — | 0 | {N(Et)₃}⁺Cl⁻ |
| A1-1011 | prazosin | N | O | O | CO₂H | O | O | 1 | CF₃CH₂ |
| A1-1012 | prazosin | N | O | O | CO₂H | O | O | 1 | cyclopropyl |
| A1-1013 | prazosin | N | O | O | CO₂H | O | O | 1 | 2-OMe-Ph |
| A1-1014 | prazosin | N | O | O | CO₂H | O | O | 1 | MeSCH₂ |
| A1-1015 | prazosin | N | O | O | CO₂H | O | O | 1 | MeOC112 |
| A1-1016 | prazosin | N | O | O | CO₂H | S | S | 1 | Ph |
| A1-1017 | prazosin | N | O | O | CO₂H | S | S | 1 | NMe₂ |
| A1-1018 | prazosin | N | O | O | CO₂H | S | S | 1 | NBn₂ |
| A1-1019 | prazosin | N | O | O | CO₂H | O | S | 1 | Ph |
| A1-1020 | prazosin | N | O | O | CO₂H | O | S | 1 | Me |
| A1-1021 | sulfamethizolee | N | O | O | CO₂H | O | O | 1 | methyl |
| A1-1022 | sulfamethizolee | N | O | O | CO₂H | O | O | 1 | C(CH₃)₃ |
| A1-1023 | sulfamethizolee | N | O | O | CO₂H | O | O | 1 | Ph |
| A1-1024 | sulfamethizolee | N | O | O | CO₂H | O | O | 1 | 4-Me-Ph |
| A1-1025 | sulfamethizolee | N | O | O | CO₂H | O | O | 1 | 2-pyridyl |
| A1-1026 | sulfamethizolee | N | O | O | CO₂H | O | O | 1 | 4-pyridyl |
| A1-1027 | sulfamethizolee | N | O | O | CO₂H | O | O | 1 | 2-furyl |
| A1-1028 | sulfamethizolee | N | O | O | CO₂H | O | O | 1 | 2-thienyl |
| A1-1029 | sulfamethizolee | N | O | O | CO₂H | — | — | 0 | Cl |
| A1-1030 | sulfamethizolee | N | O | O | CO₂H | — | — | 0 | {N(Et)₃}⁺Cl⁻ |
| A1-1031 | sulfamethizolee | N | O | O | CO₂H | O | O | 1 | CF₃CH₂ |
| A1-1032 | sulfamethizolee | N | O | O | CO₂H | O | O | 1 | cyclopropyl |
| A1-1033 | sulfamethizolee | N | O | O | CO₂H | O | O | 1 | 2-OMe-Ph |
| A1-1034 | sulfamethizolee | N | O | O | CO₂H | O | O | 1 | MeSCH₂ |
| A1-1035 | sulfamethizolee | N | O | O | CO₂H | O | O | 1 | MeOCH₂ |
| A1-1036 | sulfamethizolee | N | O | O | CO₂H | S | S | 1 | Ph |
| A1-1037 | sulfamethizolee | N | O | O | CO₂H | S | S | 1 | NMe₂ |
| A1-1038 | sulfamethizolee | N | O | O | CO₂H | S | S | 1 | NBn₂ |
| A1-1039 | sulfamethizolee | N | O | O | CO₂H | O | S | 1 | Ph |
| A1-1040 | sulfamethizolee | N | O | O | CO₂H | O | S | 1 | Me |
| A1-1041 | tacrine | N | O | O | CO₂H | O | O | 1 | methyl |
| A1-1042 | tacrine | N | O | O | CO₂H | O | O | 1 | C(CH₃)₃ |
| A1-1043 | tacrine | N | O | O | CO₂H | O | O | 1 | Ph |
| A1-1044 | tacrine | N | O | O | CO₂H | O | O | 1 | 4-Me-Ph |
| A1-1045 | tacrine | N | O | O | CO₂H | O | O | 1 | 2-pyridyl |
| A1-1046 | tacrine | N | O | O | CO₂H | O | O | 1 | 4-pyridyl |
| A1-1047 | tacrine | N | O | O | CO₂H | O | O | 1 | 2-furyl |
| A1-1048 | tacrine | N | O | O | CO₂H | O | O | 1 | 2-thienyl |
| A1-1049 | tacrine | N | O | O | CO₂H | — | — | 0 | Cl |
| A1-1050 | tacrine | N | O | O | CO₂H | — | — | 0 | {N(Et)₃}⁺Cl⁻ |
| A1-1051 | tacrine | N | O | O | CO₂H | O | O | 1 | CF₃CH₂ |
| A1-1052 | tacrine | N | O | O | CO₂H | O | O | 1 | cyclopropyl |
| A1-1053 | tacrine | N | O | O | CO₂H | O | O | 1 | 2-OMe-Ph |
| A1-1054 | tacrine | N | O | O | CO₂H | O | O | 1 | MeSCH₂ |
| A1-1055 | tacrine | N | O | O | CO₂H | O | O | 1 | MeOCH₂ |
| A1-1056 | tacrine | N | O | O | CO₂H | S | S | 1 | Ph |
| A1-1057 | tacrine | N | O | O | CO₂H | S | S | 1 | NMe₂ |
| A1-1058 | tacrine | N | O | O | CO₂H | S | S | 1 | NBn₂ |
| A1-1059 | tacrine | N | O | O | CO₂H | O | S | 1 | Ph |
| A1-1060 | tacrine | N | O | O | CO₂H | O | S | 1 | Me |
| A1-1061 | terazosin | N | O | O | CO₂H | O | O | 1 | methyl |
| A1-1062 | terazosin | N | O | O | CO₂H | O | O | 1 | C(CH₃)₃ |
| A1-1063 | terazosin | N | O | O | CO₂H | O | O | 1 | Ph |
| A1-1064 | terazosin | N | O | O | CO₂H | O | O | 1 | 4-Me-Ph |
| A1-1065 | terazosin | N | O | O | CO₂H | O | O | 1 | 2-pyridyl |
| A1-1066 | terazosin | N | O | O | CO₂H | O | O | 1 | 4-pyridyl |
| A1-1067 | terazosin | N | O | O | CO₂H | O | O | 1 | 2-furyl |
| A1-1068 | terazosin | N | O | O | CO₂H | O | O | 1 | 2-thienyl |
| A1-1069 | terazosin | N | O | O | CO₂H | — | — | 0 | Cl |
| A1-1070 | terazosin | N | O | O | CO₂H | — | — | 0 | {N(Et)₃}⁺Cl⁻ |
| A1-1071 | terazosin | N | O | O | CO₂H | O | O | 1 | CF₃CH₂ |
| A1-1072 | terazosin | N | O | O | CO₂H | O | O | 1 | cyclopropyl |
| A1-1073 | terazosin | N | O | O | CO₂H | O | O | 1 | 2-OMe-Ph |
| A1-1074 | terazosin | N | O | O | CO₂H | O | O | 1 | MeSCH₂ |
| A1-1075 | terazosin | N | O | O | CO₂H | O | O | 1 | MeOCH₂ |
| A1-1076 | terazosin | N | O | O | CO₂H | S | S | 1 | Ph |
| A1-1077 | terazosin | N | O | O | CO₂H | S | S | 1 | NMe₂ |
| A1-1078 | terazosin | N | O | O | CO₂H | S | S | 1 | NBn₂ |
| A1-1079 | terazosin | N | O | O | CO₂H | O | S | 1 | Ph |
| A1-1080 | terazosin | N | O | O | CO₂H | O | S | 1 | Me |
| A1-1081 | timolol | N | O | O | CO₂H | O | O | 1 | methyl |
| A1-1082 | timolol | N | O | O | CO₂H | O | O | 1 | O(CH₃)₃ |
| A1-1083 | timolol | N | O | O | CO₂H | O | O | 1 | Ph |
| A1-1084 | timolol | N | O | O | CO₂H | O | O | 1 | 4-Me-Ph |
| A1-1085 | timolol | N | O | O | CO₂H | O | O | 1 | 2-pyridyl |
| A1-1086 | timolol | N | O | O | CO₂H | O | O | 1 | 4-pyridyl |
| A1-1087 | timolol | N | O | O | CO₂H | O | O | 1 | 2-furyl |
| A1-1088 | timolol | N | O | O | CO₂H | O | O | 1 | 2-thienyl |
| A1-1089 | timolol | N | O | O | CO₂H | — | — | 0 | Cl |
| A1-1090 | timolol | N | O | O | CO₂H | — | — | 0 | {N(Et)₃}⁺Cl⁻ |
| A1-1091 | timolol | N | O | O | CO₂H | O | O | 1 | CF₃CH₂ |
| A1-1092 | timolol | N | O | O | CO₂H | O | O | 1 | cyclopropyl |
| A1-1093 | timolol | N | O | O | CO₂H | O | O | 1 | 2-OMe-Ph |
| A1-1094 | timolol | N | O | O | CO₂H | O | O | 1 | MeSCH₂ |
| A1-1095 | timolol | N | O | O | CO₂H | O | O | 1 | MeOCH₂ |
| A1-1096 | timolol | N | O | O | CO₂H | S | S | 1 | Ph |
| A1-1097 | timolol | N | O | O | CO₂H | S | S | 1 | NMe₂ |
| A1-1098 | timolol | N | O | O | CO₂H | S | S | 1 | NBn₂ |
| A1-1099 | timolol | N | O | O | CO₂H | O | S | 1 | Ph |
| A1-1100 | timolol | N | O | O | CO₂H | O | S | 1 | Me |
| A1-1121 | xylometazoline | N | O | O | CO₂H | O | O | 1 | methyl |
| A1-1122 | xylometazoline | N | O | O | CO₂H | O | O | 1 | C(CH₃)₃ |
| A1-1123 | xylometazoline | N | O | O | CO₂H | O | O | 1 | Ph |
| A1-1124 | xylometazoline | N | O | O | CO₂H | O | O | 1 | 4-Me-Ph |
| A1-1125 | xylometazoline | N | O | O | CO₂H | O | O | 1 | 2-pyridyl |
| A1-1126 | xylometazoline | N | O | O | CO₂H | O | O | 1 | 4-pyridyl |
| A1-1127 | xylometazoline | N | O | O | CO₂H | O | O | 1 | 2-furyl |
| A1-1128 | xylometazoline | N | O | O | CO₂H | O | O | 1 | 2-thienyl |
| A1-1129 | xylometazoline | N | O | O | CO₂H | — | — | 0 | Cl |
| A1-1130 | xylometazoline | N | O | O | CO₂H | — | — | 0 | {N(Et)₃}⁺Cl⁻ |
| A1-1131 | xylometazoline | N | O | O | CO₂H | O | O | 1 | CF₃CH₂ |
| A1-1132 | xylometazoline | N | O | O | CO₂H | O | O | 1 | cyclopropyl |
| A1-1133 | xylometazoline | N | O | O | CO₂H | O | O | 1 | 2-OMe-Ph |
| A1-1134 | xylometazoline | N | O | O | CO₂H | O | O | 1 | MeSOH₂ |
| A1-1135 | xylometazoline | N | O | O | CO₂H | O | O | 1 | MeOCH₂ |
| A1-1136 | xylometazoline | N | O | O | CO₂H | S | S | 1 | Ph |
| A1-1137 | xylometazoline | N | O | O | CO₂H | S | S | 1 | NMe₂ |
| A1-1138 | xylometazoline | N | O | O | CO₂H | S | S | 1 | NBn₂ |
| A1-1139 | xylometazoline | N | O | O | CO₂H | O | S | 1 | Ph |
| A1-1140 | xylometazoline | N | O | O | CO₂H | O | S | 1 | Me |
| A1-1141 | lovastatin | O | O | O | CO₂H | O | O | 1 | methyl |
| A1-1142 | lovastatin | O | O | O | CO₂H | O | O | 1 | C(CH₃)₃ |
| A1-1143 | lovastatin | O | O | O | CO₂H | O | O | 1 | Ph |
| A1-1144 | lovastatin | O | O | O | CO₂H | O | O | 1 | 4-Me-Ph |
| A1-1145 | lovastatin | O | O | O | CO₂H | O | O | 1 | 2-pyridyl |
| A1-1146 | lovastatin | O | O | O | CO₂H | O | O | 1 | 4-pyridyl |
| A1-1147 | lovastatin | O | O | O | CO₂H | O | O | 1 | 2-furyl |
| A1-1148 | lovastatin | O | O | O | CO₂H | O | O | 1 | 2-thienyl |
| A1-1149 | lovastatin | O | O | O | CO₂H | — | — | 0 | Cl |
| A1-1150 | lovastatin | O | O | O | CO₂H | — | — | 0 | {N(Et)₃}⁺Cl⁻ |
| A1-1151 | lovastatin | O | O | O | CO₂H | O | O | 1 | CF₃CH₂ |
| A1-1152 | lovastatin | O | O | O | CO₂H | O | O | 1 | cyclopropyl |
| A1-1153 | lovastatin | O | O | O | CO₂H | O | O | 1 | 2-OMe-Ph |
| A1-1154 | lovastatin | O | O | O | CO₂H | O | O | 1 | MeSCH₂ |
| A1-1155 | lovastatin | O | O | O | CO₂H | O | O | 1 | MeOCH₂ |
| A1-1156 | lovastatin | O | O | O | CO₂H | S | S | 1 | Ph |
| A1-1157 | lovastatin | O | O | O | CO₂H | S | S | 1 | NMe₂ |
| A1-1158 | lovastatin | O | O | O | CO₂H | S | S | 1 | NBn₂ |
| A1-1159 | lovastatin | O | O | O | CO₂H | O | S | 1 | Ph |
| A1-1160 | lovastatin | O | O | O | CO₂H | O | S | 1 | Me |
| A1-1161 | warfarin | O | O | O | CO₂H | O | O | 1 | methyl |
| A1-1162 | warfarin | O | O | O | CO₂H | O | O | 1 | C(CH₃)₃ |
| A1-1163 | warfarin | O | O | O | CO₂H | O | O | 1 | Ph |
| A1-1164 | warfarin | O | O | O | CO₂H | O | O | 1 | 4-Me-Ph |
| A1-1165 | warfarin | O | O | O | CO₂H | O | O | 1 | 2-pyridyl |
| A1-1166 | warfarin | O | O | O | CO₂H | O | O | 1 | 4-pyridyl |
| A1-1167 | warfarin | O | O | O | CO₂H | O | O | 1 | 2-furyl |
| A1-1168 | warfarin | O | O | O | CO₂H | O | O | 1 | 2-thienyl |
| A1-1169 | warfarin | O | O | O | CO₂H | — | — | 0 | Cl |
| A1-1170 | warfarin | O | O | O | CO₂H | — | — | 0 | {N(Et)₃}⁺Cl⁻ |
| A1-1171 | warfarin | O | O | O | CO₂H | O | O | 1 | CF₃CH₂ |
| A1-1172 | warfarin | O | O | O | CO₂H | O | O | 1 | cyclopropyl |
| A1-1173 | warfarin | O | O | O | CO₂H | O | O | 1 | 2-OMePh |
| A1-1174 | warfarin | O | O | O | CO₂H | O | O | 1 | MeSCH₂ |
| A1-1175 | warfarin | O | O | O | CO₂H | O | O | 1 | MeOCH₂ |
| A1-1176 | warfarin | O | O | O | CO₂H | S | S | 1 | Ph |
| A1-1177 | warfarin | O | O | O | CO₂H | S | S | 1 | NMe₂ |
| A1-1178 | warfarin | O | O | O | CO₂H | S | S | 1 | NBn₂ |
| A1-1179 | warfarin | O | O | O | CO₂H | O | S | 1 | Ph |
| A1-1180 | warfarin | O | O | O | CO₂H | O | S | 1 | Me |
| A1-1181 | atorvastatin | O | O | O | CO₂H | O | O | 1 | methyl |
| A1-1182 | atorvastatin | O | O | O | CO₂H | O | O | 1 | C(CH₃)₃ |
| A1-1183 | atorvastatin | O | O | O | CO₂H | O | O | 1 | Ph |
| A1-1184 | atorvastatin | O | O | O | CO₂H | O | O | 1 | 4-Me-Ph |
| A1-1185 | atarvastatin | O | O | O | CO₂H | O | O | 1 | 2-pyridyl |
| A1-1186 | atorvastatin | O | O | O | CO₂H | O | O | 1 | 4-pyridyl |
| A1-1187 | atorvastatin | O | O | O | CO₂H | O | O | 1 | 2-furyl |
| A1-1188 | atorvastatin | O | O | O | CO₂H | O | O | 1 | 2-thienyl |
| A1-1189 | atorvastatin | O | O | O | CO₂H | — | — | 0 | Cl |
| A1-1190 | atorvastatin | O | O | O | CO₂H | — | — | 0 | {N(Et)₃}⁺Cl⁻ |
| A1-1191 | atorvastatin | O | O | O | CO₂H | O | O | 1 | CF₃CH₂ |
| A1-1192 | atorvastatin | O | O | O | CO₂H | O | O | 1 | cyclopropyl |
| A1-1193 | atorvastatin | O | O | O | CO₂H | O | O | 1 | 2-OMe-Ph |
| A1-1194 | atorvastatin | O | O | O | CO₂H | O | O | 1 | MeSCH₂ |
| A1-1195 | atorvastatin | O | O | O | CO₂H | O | O | 1 | MeOCH₂ |
| A1-1196 | atorvastatin | O | O | O | CO₂H | S | S | 1 | Ph |
| A1-1197 | atorvastatin | O | O | O | CO₂H | S | S | 1 | NMe₂ |
| A1-1198 | atorvastatin | O | O | O | CO₂H | S | S | 1 | NBn₂ |
| A1-1199 | atorvastatin | O | O | O | CO₂H | O | S | 1 | Ph |
| A1-1200 | atorvastatin | O | O | O | CO₂H | O | S | 1 | Me |
| A1-1201 | atropine | O | O | O | CO₂H | O | O | 1 | methyl |
| A1-1202 | atropine | O | O | O | CO₂H | O | O | 1 | C(CH₃)₃ |
| A1-1203 | atropine | O | O | O | CO₂H | O | O | 1 | Ph |
| A1-1204 | atropine | O | O | O | CO₂H | O | O | 1 | 4-Me-Ph |
| A1-1205 | atropine | O | O | O | CO₂H | O | O | 1 | 2-pyridyl |
| A1-1206 | atropine | O | O | O | CO₂H | O | O | 1 | 4-pyridyl |
| A1-1207 | atropine | O | O | O | CO₂H | O | O | 1 | 2-furyl |
| A1-1208 | atropine | O | O | O | CO₂H | O | O | 1 | 2-thienyl |
| A1-1209 | atropine | O | O | O | CO₂H | — | — | 0 | Cl |
| A1-1210 | atropine | O | O | O | CO₂H | — | — | 0 | {N(Et)₃}⁺Cl⁻ |
| A1-1211 | atropine | O | O | O | CO₂H | O | O | 1 | CF₃CH₂ |
| A1-1212 | atropine | O | O | O | CO₂H | O | O | 1 | cyclopropyl |
| A1-1213 | atropine | O | O | O | CO₂H | O | O | 1 | 2-OMe-Ph |
| A1-1214 | atropine | O | O | O | CO₂H | O | O | 1 | MeSCH₂ |
| A1-1215 | atropine | O | O | O | CO₂H | O | O | 1 | MeOCH₂ |
| A1-1216 | atropine | O | O | O | CO₂H | S | S | 1 | Ph |
| A1-1217 | atropine | O | O | O | CO₂H | S | S | 1 | NMe₂ |
| A1-1218 | atropine | O | O | O | CO₂H | S | S | 1 | NBn₂ |
| A1-1219 | atropine | O | O | O | CO₂H | O | S | 1 | Ph |
| A1-1220 | atropine | O | O | O | CO₂H | O | S | 1 | Me |
| A1-1221 | bicalutamide | O | O | O | CO₂H | O | O | 1 | methyl |
| A1-1222 | bicalutamide | O | O | O | CO₂H | O | O | 1 | C(CH₃)₃ |
| A1-1223 | bicalutamide | O | O | O | CO₂H | O | O | 1 | Ph |
| A1-1224 | bicalutamide | O | O | O | CO₂H | O | O | 1 | 4-Me-Ph |
| A1-1225 | bicalutamide | O | O | O | CO₂H | O | O | 1 | 2-pyridyl |
| A1-1226 | bicalutamide | O | O | O | CO₂H | O | O | 1 | 4-pyridyl |
| A1-1227 | bicalutamide | O | O | O | CO₂H | O | O | 1 | 2-furyl |
| A1-1228 | bicalutamide | O | O | O | CO₂H | O | O | 1 | 2-thienyl |
| A1-1229 | bicalutamide | O | O | O | CO₂H | — | — | 0 | Cl |
| A1-1230 | bicalutamide | O | O | O | CO₂H | — | — | 0 | {N(Et)₃}⁺Cl⁻ |
| A1-1231 | bicalutamide | O | O | O | CO₂H | O | O | 1 | CF₃CH₂ |
| A1-1232 | bicalutamide | O | O | O | CO₂HI | O | O | 1 | cyclopropyl |
| A1-1233 | bicalutamide | O | O | O | CO₂H | O | O | 1 | 2-OMe-Ph |
| A1-1234 | bicalutamide | O | O | O | CO₂H | O | O | 1 | MeSCH₂ |
| AI-1235 | bicalutamide | O | O | O | CO₂H | O | O | 1 | MeOCH₂ |
| A1-1236 | bicalutamide | O | O | O | CO₂H | 5 | S | 1 | Ph |
| A1-1237 | bicalutamide | O | O | O | CO₂H | S | S | 1 | NMe₂ |
| A1-1238 | bicalutamide | O | O | O | CO₂H | S | S | 1 | NBn₂ |
| A1-1239 | bicalutamide | O | O | O | CO₂H | O | S | 1 | Ph |
| A1-1240 | bicalutamide | O | O | O | CO₂H | O | S | 1 | Me |
| A1-1241 | clobesol | O | O | O | CO₂H | O | O | 1 | methyl |
| A1-1242 | clobesol | O | O | O | CO₂H | O | O | 1 | C(CH₃)₃ |
| A1-1243 | clobesol | O | O | O | CO₂H | O | O | 1 | Ph |
| A1-1244 | clobesol | O | O | O | CO₂H | O | O | 1 | 4-Me-Ph |
| A1-1245 | clobesol | O | O | O | CO₂H | O | O | 1 | 2-pyridyl |
| A1-1246 | clobesol | O | O | O | CO₂H | O | O | 1 | 4-pyridyl |
| A1-1247 | clobesol | O | O | O | CO₂H | O | O | 1 | 2-furyl |
| A1-1248 | clobesol | O | O | O | CO₂H | O | O | 1 | 2-thienyl |
| A1-1249 | clobesol | O | O | O | CO₂H | — | — | 0 | Cl |
| A1-1250 | clobesol | O | O | O | CO₂H | — | — | {N(Et)₃}⁺Cl⁻ | |
| A1-1251 | clobesol | O | O | O | CO₂H | O | O | 1 | CF₃CH₂ |
| A1-1252 | clobesol | O | O | O | CO₂H | O | O | 1 | cyclopropyl |
| A1-1253 | clobesol | O | O | O | CO₂H | O | O | 1 | 2-OMe-Ph |
| A1-1254 | clobesol | O | O | O | CO₂H | O | O | 1 | MeSCH₂ |
| A1-1255 | clobesol | O | O | O | CO₂H | O | O | 1 | MeOCH₂ |
| A1-1256 | clobesol | O | O | O | CO₂H | S | S | 1 | Ph |
| A1-1257 | clobesol | O | O | O | CO₂H | S | S | 1 | NMe₂ |
| A1-1258 | clobesol | O | O | O | CO₂H | S | S | 1 | NBn₂ |
| A1-1259 | clobesol | O | O | O | CO₂H | O | S | 1 | Ph |
| A1-1260 | clobesol | O | O | O | CO₂H | O | S | 1 | Me |
| A1-1261 | fluticasone | O | O | O | CO₂H | O | O | 1 | methyl |
| | propionate | | | | | | | | |
| A1-1262 | fluticasone | O | O | O | CO₂H | O | O | 1 | C(CH₃)₃ |
| | propioriate | | | | | | | | |
| A1-1263 | fluticasone | O | O | O | CO₂H | O | O | 1 | Ph |
| | propionate | | | | | | | | |
| A1-1264 | fluticasone | O | O | O | CO₂H | O | O | 1 | 4-Me-Ph |
| | propionate | | | | | | | | |
| A1-1265 | fluticasone | O | O | O | CO₂H | O | O | 1 | 2-pyridyl |
| | propionate | | | | | | | | |
| A1-1266 | fluticasone | O | O | O | CO₂H | O | O | 1 | 4-pyridyl |
| | propionate | | | | | | | | |
| A1-1267 | fluticasone | O | O | O | CO₂H | O | O | 1 | 2-furyl |
| | propionate | | | | | | | | |
| A1-1268 | fluticasone | O | O | O | CO₂H | O | O | 1 | 2-thienyl |
| | propionate | | | | | | | | |
| A1-1269 | fluticasone | O | O | O | CO₂H | — | — | 0 | Cl |
| | propionate | | | | | | | | |
| A1-1270 | fluticasone | O | O | O | CO₂H | — | — | 0 | {N(Et)₃}⁺Cl⁻ |
| | propionate | | | | | | | | |
| A1-1271 | fluticasone | O | O | O | CO₂H | O | O | 1 | CF₃CH₂ |
| | propionate | | | | | | | | |
| A1-1272 | fluticasone | O | O | O | CO₂H | O | O | 1 | cyclopropyl |
| | propionate | | | | | | | | |
| A1-1273 | fluticasone | O | O | O | CO₂H | O | O | 1 | 2-OMe-Ph |
| | propionate | | | | | | | | |
| A1-1274 | fluticasone | O | O | O | CO₂H | O | O | 1 | MeSCH₂ |
| | propionate | | | | | | | | |
| A1-1275 | fluticasone | O | O | O | CO₂H | O | O | 1 | MeOCH₂ |
| | propionate | | | | | | | | |
| A1-1276 | fluticasone | O | O | O | CO₂H | S | S | 1 | Ph |
| | propionate | | | | | | | | |
| A1-1277 | fluticasone | O | O | O | CO₂H | S | S | 1 | NMe₂ |
| | propionate | | | | | | | | |
| A1-1278 | fluticasone | O | O | O | CO₂H | S | S | 1 | NBn₂ |
| | propionate | | | | | | | | |
| A1-1279 | fluticasone | O | O | O | CO₂H | O | S | 1 | Ph |
| | propionate | | | | | | | | |
| A1-1280 | fluticasone | O | O | O | CO₂H | O | S | 1 | Me |
| | propionate | | | | | | | | |
| A1-1301 | loperidine HCl | O | O | O | CO₂H | O | O | 1 | methyl |
| A1-1302 | loperidine HCl | O | O | O | CO₂H | O | O | 1 | C(CH₃)₃ |
| A1-1303 | loperidine HCl | O | O | O | CO₂H | O | O | 1 | Ph |
| A1-1304 | loperidine HCl | O | O | O | CO₂H | O | O | 1 | 4-Me-Ph |
| A1-1305 | loperidine HCl | O | O | O | CO₂H | O | O | 1 | 2-pyridyl |
| A1-1306 | loperidine HCl | O | O | O | CO₂H | O | O | 1 | 4-pyridyl |
| AI-1307 | loperidine HCl | O | O | O | CO₂H | O | O | 1 | 2-furyl |
| A1-1308 | loperidine HCl | O | O | O | CO₂H | O | O | 1 | 2-thienyl |
| A1-1309 | loperidine HCl | O | O | O | CO₂H | — | — | 0 | Cl |
| A1-1310 | loperidine HCl | O | O | O | CO₂H | — | — | 0 | {N(Et)₃}⁺Cl⁻ |
| A1-1311 | loperidine HCl | O | O | O | CO₂H | O | O | 1 | CF₃CH₂ |
| A1-1312 | loperidine HCl | O | O | O | CO₂H | O | O | 1 | cyclopropyl |
| A1-1313 | loperidine HCl | O | O | O | CO₂H | O | O | 1 | 2-OMe-Ph |
| A1-1314 | loperidine HCl | O | O | O | CO₂H | O | O | 1 | MeSCH₂ |
| A1-1315 | loperidine HCl | O | O | O | CO₂H | O | O | 1 | MeOCH₂ |
| A1-1316 | loperidine HCl | O | O | O | CO₂H | S | S | 1 | Ph |
| A1-1317 | loperidine HCl | O | O | O | CO₂H | S | S | 1 | NMe₂ |
| A1-1318 | loperidine HCl | O | O | O | CO₂H | S | S | 1 | NBn₂ |
| A1-1319 | loperidine HCl | O | O | O | CO₂H | O | S | 1 | Ph |
| A1-1320 | loperidine HCl | O | O | O | CO₂H | O | S | 1 | Me |
| A1-1321 | monozid | O | O | O | CO₂H | O | O | 1 | methyl |
| A1-1322 | monozid | O | O | O | CO₂H | O | O | 1 | C(CH₃)₃ |
| A1-1323 | monozid | O | O | O | CO₂H | O | O | 1 | Ph |
| A1-1324 | monozid | O | O | O | CO₂H | O | O | 1 | 4-Me-Ph |
| A1-1325 | monozid | O | O | O | CO₂H | O | O | 1 | 2-pyridyl |
| A1-1326 | monozid | O | O | O | CO₂H | O | O | 1 | 4-pyridyl |
| A1-1327 | monozid | O | O | O | CO₂H | .0 | O | 1 | 2-furyl |
| A1-1328 | monozid | O | O | O | CO₂H | O | O | 1 | 2-thienyl |
| A1-1329 | monozid | O | O | O | CO₂H | — | — | 0 | Cl |
| A1-1330 | monozid | O | O | O | CO₂H | — | — | 0 | {N(Et)₃}⁺Cl⁻ |
| A1-1331 | monozid | O | O | O | CO₂H | O | O | 1 | CF₃CH₂ |
| A1-1332 | monozid | O | O | O | CO₂H | O | O | 1 | cyclopropyl |
| A1-1333 | monozid | O | O | O | CO₂H | O | O | 1 | 2-OMe-Ph |
| A1-1334 | monozid | O | O | O | CO₂H | O | O | 1 | MeSCH₂ |
| A1-1335 | monozid | O | O | O | CO₂H | O | O | 1 | MeOCH₂ |
| A1-1336 | monozid | O | O | O | CO₂H | S | S | 1 | Ph |
| A1-1337 | monozid | O | O | O | CO₂H | S | S | 1 | NMe₂ |
| A1-1338 | monozid | O | O | O | CO₂H | S | S | 1 | NBn₂ |
| A1-1339 | monozid | O | O | O | CO₂H | O | S | 1 | Ph |
| A1-1340 | monozid | O | O | O | CO₂H | O | S | 1 | Me |
| A1-1341 | oxybutynin HCl | O | O | O | CO₂H | O | O | 1 | methyl |
| A1-1342 | oxybutynin HCl | O | O | O | CO₂H | O | O | 1 | C(CH₃)₃ |
| A1-1343 | oxybutynin HCl | O | O | O | CO₂H | O | O | 1 | Ph |
| A1-1344 | oxybutynin HCl | O | O | O | CO₂H | O | O | 1 | 4-Me-Ph |
| A1-1345 | oxybutynin HCl | O | O | O | CO₂H | O | O | 1 | 2-pyridyl |
| A1-1346 | oxybutynin HCl | O | O | O | CO₂H | O | O | 1 | 4-pyridyl |
| AI-1347 | oxybutynin HCl | O | O | O | CO₂H | O | O | 1 | 2-furyl |
| A1-1348 | oxybutynin HCl | O | O | O | CO₂H | O | O | 1 | 2-thienyl |
| A1-1349 | oxybutynin HCl | O | O | O | CO₂H | — | — | 0 | Cl |
| A1-1350 | oxybutynin HCl | O | O | O | CO₂H | — | — | 0 | {N(Et)₃}⁺Cl⁻ |
| A1-1351 | oxybutynin HCl | O | O | O | CO₂H | O | O | 1 | CF₃CH₂ |
| A1-1352 | oxybutynin HCl | O | O | O | CO₂H | O | O | 1 | cyclopropyl |
| A1-1353 | oxybutynin HCl | O | O | O | CO₂H | O | O | 1 | 2-OMe-Ph |
| A1-1354 | oxybutynin HCl | O | O | O | CO₂H | O | O | 1 | MeSCH₂ |
| A1-1355 | oxybutynin HCl | O | O | O | CO₂H | O | O | 1 | MeOCH₂ |
| A1-1356 | oxybutynin HCl | O | O | O | CO₂H | S | S | 1 | Ph |
| A1-1357 | oxybutynin HCl | O | O | O | CO₂H | S | S | 1 | NMe₂ |
| A1-1358 | oxybutynin HCl | O | O | O | CO₂H | S | S | 1 | NBn₂ |
| A1-1359 | oxybutynin HCl | O | O | O | CO₂H | O | S | 1 | Ph |
| A1-1360 | oxybutynin HCl | O | O | O | CO₂H | O | S | 1 | Me |
| A1-1361 | oxycodone | O | O | O | CO₂H | O | O | 1 | methyl |
| A1-1362 | oxycodone | O | O | O | CO₂H | O | O | 1 | C(CH₃)₃ |
| A1-1363 | oxycodone | O | O | O | CO₂H | O | O | 1 | Ph |
| A1-1364 | oxycodone | O | O | O | CO₂H | O | O | 1 | 4-Me-Ph |
| A1-1365 | oxycodone | O | O | O | CO₂H | O | O | 1 | 2-pyridyl |
| A1-1366 | oxycodone | O | O | O | CO₂H | O | O | 1 | 4-pyridyl |
| A1-1367 | oxycodone | O | O | O | CO₂H | O | O | 1 | 2-furyl |
| A1-1368 | oxycodone | O | O | O | CO₂H | O | O | 1 | 2-thienyl |
| A1-1369 | oxycodone | O | O | O | CO₂H | — | — | 0 | Cl |
| A1-1370 | oxycodone | O | O | O | CO₂H | — | — | 0 | {N(Et)₃}⁺Cl⁻ |
| A1-1371 | oxycodone | O | O | O | CO₂H | O | O | 1 | CF₃CH₂ |
| A1-1372 | oxycodone | O | O | O | CO₂H | O | O | 1 | cyclopropyl |
| A1-1373 | oxycodone | O | O | O | CO₂H | O | O | 1 | 2-OMe-Ph |
| A1-1374 | oxycodone | O | O | O | CO₂H | O | O | 1 | MeSCH₂ |
| A1-1375 | oxycodone | O | O | O | CO₂H | O | O | 1 | MeOCH₂ |
| A1-1376 | oxycodone | O | O | O | CO₂H | S | S | 1 | Ph |
| A1-1377 | oxycodone | O | O | O | CO₂H | S | S | 1 | NMe₂ |
| A1-1378 | oxycodone | O | O | O | CO₂H | S | S | 1 | NBn₂ |
| A1-1379 | oxycodone | O | O | O | CO₂H | O | S | 1 | Ph |
| A1-1380 | oxycodone | O | O | O | CO₂H | O | S | 1 | Me |
| A1-1381 | piroxicam | O | O | O | CO₂H | O | O | 1 | methyl |
| A1-1382 | piroxicam | O | O | O | CO₂H | O | O | 1 | C(CH₃)₃ |
| A1-1383 | piroxicam | O | O | O | CO₂H | O | O | 1 | Ph |
| A1-1384 | piroxicam | O | O | O | CO₂H | O | O | 1 | 4-Me-Ph |
| A1-1385 | piroxicam | O | O | O | CO₂H | O | O | 1 | 2-pyridyl |
| A1-1386 | piroxicam | O | O | O | CO₂H | O | O | 1 | 4-pyridyl |
| A1-1387 | piroxicam | O | O | O | CO₂H | O | O | 1 | 2-furyl |
| A1-1388 | piroxicam | O | O | O | CO₂H | O | O | 1 | 2-thienyl |
| A1-1389 | piroxicam | O | O | O | CO₂H | — | — | 0 | Cl |
| A1-1390 | piroxicam | O | O | O | CO₂H | — | — | 0 | {N(Et)₃}⁺Cl⁻ |
| A1-1391 | piroxicam | O | O | O | CO₂H | O | O | 1 | CF₃CH₂ |
| A1-1392 | piroxicam | O | O | O | CO₂H | O | O | 1 | cyclopropyl |
| A1-1393 | piroxicam | O | O | O | CO₂H | O | O | 1 | 2-OMe-Ph |
| A1-1394 | piroxicam | O | O | O | CO₂H | O | O | 1 | MeSCH₂ |
| A1-1395 | piroxicam | O | O | O | CO₂H | O | O | 1 | MeOCH₂ |
| A1-1396 | piroxicam | O | O | O | CO₂H | S | S | 1 | Ph |
| A1-1397 | piroxicam | O | O | O | CO₂H | S | S | 1 | NMe₂ |
| A1-1398 | piroxicam | O | O | O | CO₂H | S | S | 1 | NBn₂ |
| A1-1399 | piroxicam | O | O | O | CO₂H | O | S | 1 | Ph |
| A1-1400 | piroxicam | O | O | O | CO₂H | O | S | 1 | Me |
| A1-1401 | simvastatin | O | O | O | CO₂H | O | O | 1 | methyl |
| AI-1402 | simvastatin | O | O | O | CO₂H | O | O | 1 | C(CH₃)₃ |
| A1-1403 | simvastatin | O | O | O | CO₂H | O | O | 1 | Ph |
| A1-1404 | simvastatin | O | O | O | CO₂H | O | O | 1 | 4-Me-Ph |
| A1-1405 | simvastatin | O | O | O | CO₂H | O | O | 1 | 2-pyridyl |
| A1-1406 | simvastatin | O | O | O | CO₂H | O | O | 1 | 4-pyridyl |
| A1-1407 | simvastatin | O | O | O | CO₂H | O | O | 1 | 2-furyl |
| A1-1408 | simvastatin | O | O | O | CO₂H | O | O | 1 | 2-thienyl |
| A1-1409 | simvastatin | O | O | O | CO₂H | — | — | 0 | Cl |
| A1-1410 | simvastatin | O | O | O | CO₂H | — | — | 0 | {N(Et)₃}⁺Cl⁻ |
| A1-1411 | simvastatin | O | O | O | CO₂H | O | O | 1 | CF₃OH₂ |
| A1-1412 | simvastatin | O | O | O | CO₂H | O | O | 1 | cyclopropyl |
| A1-1413 | simvastatin | O | O | O | CO₂H | O | O | 1 | 2-OMe-Ph |
| A1-1414 | simvastatin | O | O | O | CO₂H | O | O | 1 | MeSCH₂ |
| A1-1415 | simvastatin | O | O | O | CO₂H | O | O | 1 | MeOOH₂ |
| A1-1416 | simvastatin | O | O | O | CO₂H | S | S | 1 | Ph |
| A1-1417 | simvastatin | O | O | O | CO₂H | S | S | 1 | NMe₂ |
| A1-1418 | simvastatin | O | O | O | CO₂H | S | S | 1 | NBn₂ |
| A1-1419 | simvastatin | O | O | O | CO₂H | O | S | 1 | Ph |
| A1-1420 | simvastatin | O | O | O | CO₂H | O | S | 1 | Me |
| A1-1421 | troglitazone | O | O | O | CO₂H | O | O | 1 | methyl |
| A1-1422 | troglitazone | O | O | O | CO₂H | O | O | 1 | C(CH₃)a |
| A1-1423 | troglitazone | O | O | O | CO₂H | O | O | 1 | Ph |
| A1-1424 | troglitazone | O | O | O | CO₂H | O | O | 1 | 4-Me-Ph |
| A1-1425 | troglitazone | O | O | O | CO₂H | O | O | 1 | 2-pyridyl |
| A1-1426 | troglitazone | O | O | O | CO₂H | O | O | 1 | 4-pyridyl |
| A1-1427 | troglitazone | O | O | O | CO₂H | O | O | 1 | 2-furyl |
| A1-1428 | troglitazone | O | O | O | CO₂H | O | O | 1 | 2-thienyl |
| A1-1429 | troglitazone | O | O | O | CO₂H | — | — | 0 | Cl |
| A1-1430 | troglitazone | O | O | O | CO₂H | — | — | 0 | {N(Et)₃}⁺Cl⁻ |
| A1-1431 | troglitazone | O | O | O | CO₂H | O | O | 1 | CF₃CH₂ |
| A1-1432 | troglitazone | O | O | O | CO₂H | O | O | 1 | cyclopropyl |
| A1-1433 | troglitazone | O | O | O | CO₂H | O | O | 1 | 2-OMe-Ph |
| A1-1434 | troglitazone | O | O | O | CO₂H | O | O | 1 | MeSCH₂ |
| A1-1435 | troglitazone | O | O | O | CO₂H | O | O | 1 | MeOCH₂ |
| A1-1436 | troglitazone | O | O | O | CO₂H | S | S | 1 | Ph |
| A1-1437 | troglitazone | O | O | O | CO₂H | S | S | 1 | NMe₂ |
| A1-1438 | troglitazone | O | O | O | CO₂H | S | S | 1 | NBn₂ |
| A1-1439 | troglitazone | O | O | O | CO₂H | O | S | 1 | Ph |
| A1-1440 | troglitazone | O | O | O | CO₂H | O | S | 1 | Me |
| A1-1441 | carbamazepam | N | O | O | CO₂H | O | O | 1 | methyl |
| A1-1442 | carbamazepam | N | O | O | CO₂H | O | O | 1 | C(CH₃)₃ |
| A1-1443 | carbamazepam | N | O | O | CO₂H | O | O | 1 | Ph |
| A1-1444 | carbamazepam | N | O | O | CO₂H | O | O | 1 | 4-Me-Ph |
| A1-1445 | carbamazepam | N | O | O | CO₂H | O | O | 1 | 2-pyridyl |
| A1-1446 | carbamazepam | N | O | O | CO₂H | O | O | 1 | 4-pyridyl |
| A1-1447 | carbamazepam | N | O | O | CO₂H | O | O | 1 | 2-furyl |
| A1-1448 | carbamazepam | N | O | O | CO₂H | O | O | 1 | 2-thienyl |
| A1-1449 | carbamazepam | N | O | O | CO₂H | — | — | 0 | Cl |
| A1-1450 | carbamazepam | N | O | O | CO₂H | — | — | 0 | {N(Et)₃}⁺Cl⁻ |
| A1-1451 | carbamazepam | N | O | O | CO₂H | O | O | 1 | CF₃CH₂ |
| A1-1452 | carbamazepam | N | O | O | CO₂H | O | O | 1 | cyclopropyl |
| A1-1453 | carbamazepam | N | O | O | CO₂H | O | O | 1 | 2-OMePh |
| A1-1454 | carbamazepam | N | O | O | CO₂H | O | O | 1 | MeSOH₂ |
| A1-1455 | carbamazepam | N | O | O | CO₂H | O | O | 1 | MeOCH₂ |
| A1-1456 | carbamazepam | N | O | O | CO₂H | S | S | 1 | Ph |
| A1-1457 | carbamazepam | N | O | O | CO₂H | S | S | 1 | NMe₂ |
| A1-1458 | carbamazepam | N | O | O | CO₂H | S | S | 1 | NBn₂ |
| A1-1459 | carbamazepam | N | O | O | CO₂H | O | S | 1 | Ph |
| A1-1460 | carbamazepam | N | O | O | CO₂H | O | S | 1 | Me |
| A1-1461 | amoxicillin | N | O | O | CO₂H | O | O | 1 | methyl |
| A1-1462 | amoxicillin | N | O | O | CO₂H | O | O | 1 | C(CH₃)₃ |
| A1-1463 | amoxicillin | N | O | O | COH | O | O | 1 | Ph |
| A1-1464 | amoxicillin | N | O | O | CO₂H | O | O | 1 | 4-Me-Ph |
| A1-1465 | amoxicillin | N | O | O | CO₂H | O | O | 1 | 2-pyridyl |
| A1-1466 | amoxicillin | N | O | O | CO₂H | O | O | 1 | 4-pyridyl |
| A1-1467 | amoxicillin | N | O | O | CO₂H | O | O | 1 | 2-furyl |
| A1-1468 | amoxicillin | N | O | O | CO₂H | O | O | 1 | 2-thienyl |
| A1-1469 | amoxicillin | N | O | O | CO₂H | — | — | 0 | Cl |
| A1-1470 | amoxicillin | N | O | O | CO₂H | — | — | 0 | {N(Et)₃}⁺Cl⁻ |
| A1-1471 | amoxicillin | N | O | O | CO₂H | O | O | 1 | CF₃CH₂ |
| A1-1472 | amoxicillin | N | O | O | CO₂H | O | O | 1 | cyclopropyl |
| A1-1473 | amoxicillin | N | O | O | CO₂H | O | O | 1 | 2-OMe-Ph |
| A1-1474 | amoxicillin | N | O | O | CO₂H | O | O | 1 | MeSCH₂ |
| A1-1475 | amoxicillin | N | O | O | CO₂H | O | O | 1 | MeOCH₂ |
| A1-1476 | amoxicillin | N | O | O | CO₂H | S | S | 1 | Ph |
| A1-1477 | amoxicillin | N | O | O | CO₂H | S | S | 1 | NMe₂ |
| A1-1478 | amoxicillin | N | O | O | CO₂H | S | S | 1 | NBn₂ |
| A1-1479 | amoxicihin | N | O | O | CO₂H | O | S | 1 | Ph |
| A1-1480 | amoxicillin | N | O | O | CO₂H | O | S | 1 | Me |
| A1-1481 | benazepril | N | O | O | CO₂H | O | O | 1 | methyl |
| A1-1482 | benazepril | N | O | O | CO₂H | O | O | 1 | C(CH₃)₃ |
| A1-1483 | benazepril | N | O | O | CO₂H | O | O | 1 | Ph |
| A1-1484 | benazepril | N | O | O | CO₂H | O | O | 1 | 4-Me-Ph |
| A1-1485 | benazepril | N | O | O | CO₂H | O | O | 1 | 2-pyridyl |
| A1-1486 | benazepril | N | O | O | CO₂H | O | O | 1 | 4-pyridyl |
| A1-1487 | benazepril | N | O | O | CO₂H | O | O | 1 | 2-furyl |
| A1-1488 | benazepril | N | O | O | CO₂H | O | O | 1 | 2-thienyl |
| A1-1489 | benazepril | N | O | O | CO₂H | — | — | 0 | Cl |
| A1-1490 | benazepril | N | O | O | CO₂H | — | — | 0 | {N(Et)₃}⁺Cl⁻ |
| A1-1491 | benazepril | N | O | O | CO₂H | O | O | 1 | CF₃CH₂ |
| A1-1492 | benazepril | N | O | O | CO₂H | O | O | 1 | cyclopropyl |
| A1-1493 | benazepril | N | O | O | CO₂H | O | O | 1 | 2-OMe-Ph |
| A1-1494 | benazepril | N | O | O | CO₂H | O | O | 1 | MeSCH₂ |
| A1-1495 | benazepril | N | O | O | CO₂H | O | O | 1 | MeOCH₂ |
| A1-1496 | benazepril | N | O | O | CO₂H | S | S | 1 | Ph |
| A1-1497 | benazepril | N | O | O | CO₂H | S | S | 1 | NMe₂ |
| A1-1498 | benazepril | N | O | O | CO₂H | S | S | 1 | NBn₂ |
| A1-1499 | benazepnil | N | O | O | CO₂H | O | S | 1 | Ph |
| A1-1500 | benazepril | N | O | O | CO₂H | O | S | 1 | Me |
| A1-1501 | cefachlor | N | O | O | CO₂H | O | O | 1 | methyl |
| A1-1502 | cefachlor | N | O | O | CO₂H | O | O | 1 | C(CH₃)a |
| A1-1503 | cefachlor | N | O | O | CO₂H | O | O | 1 | Ph |
| A1-1504 | cefachlor | N | O | O | CO₂H | O | O | 1 | 4-Me-Ph |
| A1-1505 | cefachlor | N | O | O | CO₂H | O | O | 1 | 2-pyridyl |
| A1-1506 | cefachlor | N | O | O | CO₂H | O | O | 1 | 4-pynidyl |
| A1-1507 | cefachlor | N | O | O | CO₂H | O | O | 1 | 2-furyl |
| A1-1508 | cefachlor | N | O | O | CO₂H | O | O | 1 | 2-thienyl |
| A1-1509 | cefachlor | N | O | O | CO₂H | — | — | 0 | Cl |
| A1-1510 | cefachlor | N | O | O | CO₂H | — | — | 0 | {N(Et)₃}⁺Cl⁻ |
| A1-1511 | cefachlor | N | O | O | CO₂H | O | O | 1 | CF₃CH₂ |
| A1-1512 | cefachlor | N | O | O | CO₂H | O | O | 1 | cyclopropyl |
| A1-1513 | cefachlor | N | O | O | CO₂H | O | O | 1 | 2-OMe-Ph |
| A1-1514 | cefachlor | N | O | O | CO₂H | O | O | 1 | MeSCH₂ |
| A1-1515 | cefachlor | N | O | O | CO₂H | O | O | 1 | MeOCH₂ |
| A1-1516 | cefachlor | N | O | O | CO₂H | S | S | 1 | Ph |
| A1-1517 | cefachlor | N | O | O | CO₂H | S | S | 1 | NMe₂ |
| A1-1518 | cefachlor | N | O | O | CO₂H | S | S | 1 | NBn₂ |
| A1-1519 | cefachlor | N | O | O | CO₂H | O | S | 1 | Ph |
| A1-1520 | cefachlor | N | O | O | CO₂H | O | S | 1 | Me |
| A1-1521 | cetirizine | N | O | O | CO₂H | O | O | 1 | methyl |
| A1-1522 | cetirizine | N | O | O | CO₂H | O | O | 1 | C(CH₃)₃ |
| A1-1523 | cetirizine | N | O | O | CO₂H | O | O | 1 | Ph |
| A1-1524 | cetirizine | N | O | O | CO₂H | O | O | 1 | 4-Me-Ph |
| A1-1525 | cetirizine | N | O | O | CO₂H | O | O | 1 | 2-pyridyl |
| A1-1526 | cetirizine | N | O | O | CO₂H | O | O | 1 | 4-pyridyl |
| A1-1527 | cetirizine | N | O | O | CO₂H | O | O | 1 | 2-furyl |
| A1-1528 | cetirizine | N | O | O | CO₂H | O | O | 1 | 2-thienyl |
| A1-1529 | cetirizinec | N | O | O | CO₂H | — | — | 0 | Cl |
| A1-1530 | cetirizine | N | O | O | CO₂H | — | — | 0 | {N(Et)₃}⁺Cl⁻ |
| A1-1531 | cetirizine | N | O | O | CO₂H | O | O | 1 | CF₃CH₂ |
| A1-1532 | cetirizine | N | O | O | CO₂H | O | O | 1 | cyclopropyl |
| A1-1533 | cetirizine | N | O | O | CO₂H | O | O | 1 | 2-OMe-Ph |
| Ai-1534 | cetirizine | N | O | O | CO₂H | O | O | 1 | MeSCH₂ |
| A1-1535 | cetirizine | N | O | O | CO₂H | O | O | 1 | MeOCH₂ |
| A1-1536 | cetirizine | N | O | O | CO₂H | S | S | 1 | Ph |
| A1-1537 | cetirizine | N | O | O | CO₂H | S | S | 1 | NMe₂ |
| A1-1538 | cetirizine | N | O | O | CO₂H | S | S | 1 | NBn₂ |
| A1-1539 | cetirizine | N | O | O | CO₂H | O | S | 1 | Ph |
| A1-1540 | cetirizine | N | O | O | CO₂H | O | S | 1 | Me |
| A1-1541 | cefadroxil | N | O | O | CO₂H | O | O | 1 | methyl |
| A1-1542 | cefadroxil | N | O | O | CO₂H | O | O | 1 | C(CH₃)₃ |
| A1-1543 | cefadroxil | N | O | O | CO₂H | O | O | 1 | Ph |
| A1-1544 | cefadroxil | N | O | O | CO₂H | O | O | 1 | 4-Me-Ph |
| A1-1545 | cefadroxil | N | O | O | CO₂H | O | O | 1 | 2-pyridyl |
| A1-1546 | cefadroxil | N | O | O | CO₂H | O | O | 1 | 4-pyridyl |
| A1-1547 | cefadroxil | N | O | O | CO₂H | O | O | 1 | 2-furyl |
| A1-1548 | cefadroxil | N | O | O | CO₂H | O | O | 1 | 2-thienyl |
| A1-1549 | cefadroxil | N | O | O | CO₂H | — | — | 0 | Cl |
| A1-1550 | cefadroxil | N | O | O | CO₂H | — | — | 0 | {N(Et)₃}⁺Cl⁻ |
| A1-1551 | cefadroxil | N | O | O | CO₂H | O | O | 1 | CF₃CH₂ |
| A1-1552 | cefadroxil | N | O | O | CO₂H | O | O | 1 | cyclopropyl |
| A1-1553 | cefadroxil | N | O | O | CO₂H | O | O | 1 | 2-OMe-Ph |
| A1-1554 | cefadroxil | N | O | O | CO₂H | O | O | 1 | MeSCH₂ |
| A1-1555 | cefadroxil | N | O | O | CO₂H | O | O | 1 | MeOCH₂ |
| A1-1556 | cefadroxil | N | O | O | CO₂H | S | S | 1 | Ph |
| A1-1557 | cefadroxil | N | O | O | CO₂H | S | S | 1 | NMe₂ |
| A1-1558 | cefadroxil | N | O | O | CO₂H | S | S | 1 | NBn₂ |
| A1-1559 | cefadroxil | N | O | O | CO₂H | O | S | 1 | Ph |
| A1-1560 | cefadroxil | N | O | O | CO₂H | O | S | 1 | Me |
| A1-1561 | clonazepam | N | O | O | CO₂H | O | O | 1 | methyl |
| A1-1562 | clonazepam | N | O | O | CO₂H | O | O | 1 | C(CH₃)₃ |
| A1-1563 | clonazepam | N | O | O | CO₂H | O | O | 1 | Ph |
| A1-1564 | clonazepam | N | O | O | CO₂H | O | O | 1 | 4-Me-Ph |
| AI-1565 | clonazepam | N | O | O | CO₂H | O | O | 1 | 2-pyridyl |
| A1-1566 | clonazepam | N | O | O | CO₂H | O | O | 1 | 4-pyridyl |
| A1-1567 | clonazepam | N | O | O | CO₂H | O | O | 1 | 2-furyl |
| A1-1568 | clonazepam | N | O | O | CO₂H | O | O | 1 | 2-thienyl |
| A1-1569 | clonazepam | N | O | O | CO₂H | — | — | 0 | Cl |
| A1-1570 | clonazepam | N | O | O | CO₂H | — | — | 0 | {N(Et)₃}⁺Cl⁻ |
| A1-1571 | clonazepam | N | O | O | CO₂H | O | O | 1 | CF₃CH₂ |
| A1-1572 | clonazepam | N | O | O | CO₂H | O | O | 1 | cyclopropyl |
| A1-1573 | clonazepam | N | O | O | COY | O | O | 1 | 2-OMe-Ph |
| A1-1574 | clonazepam | N | O | O | CO₂H | O | O | 1 | MeSCH₂ |
| A1-1575 | clonazepam | N | O | O | CO₂H | O | O | 1 | MeOCH₂ |
| A1-1576 | clonazepam | N | O | O | CO₂H | S | S | 1 | Ph |
| A1-1577 | clonazepam | N | O | O | CO₂H | S | S | 1 | NMe₂ |
| A1-1578 | clonazepam | N | O | O | CO₂H | S | S | 1 | NBn₂ |
| A1-1579 | clonazepam | N | O | O | CO₂H | O | S | 1 | Ph |
| A1-1580 | clonazepain | N | O | O | CO₂H | O | S | 1 | Me |
| A1-1581 | demethylimipramine | N | O | O | CO₂H | O | O | 1 | methyl |
| A1-1582 | demethylimipramine | N | O | O | CO₂H | O | O | 1 | C(CH₃)₃ |
| A1-1583 | demethylimipramine | N | O | O | CO₂H | O | O | 1 | Ph |
| A1-1584 | demethylimipramine | N | O | O | CO₂H | O | O | 1 | 4-Me-Ph |
| A1-1585 | demethylimipramine | N | O | O | CO₂H | O | O | 1 | 2-pyridyl |
| A1-1586 | demethylimipramine | N | O | O | CO₂H | O | O | 1 | 4-pyridyl |
| A1-1587 | demethylimipramine | N | O | O | CO₂H | O | O | 1 | 2-furyl |
| A1-1588 | demethylimipramine | N | O | O | CO₂H | O | O | 1 | 2-thienyl |
| A1-1589 | demethylimipramine | N | O | O | CO₂H | — | — | 0 | Cl |
| A1-1590 | demethylimipramine | N | O | O | CO₂H | — | — | 0 | {N(Et)₃}⁺Cl⁻ |
| A1-1591 | demethylimipramine | N | O | O | CO₂H | O | O | 1 | CF₃CH₂ |
| A1-1592 | demethylimipramine | N | O | O | CO₂H | O | O | 1 | cyclopropyl |
| A1-1593 | demethylimipramine | N | O | O | CO₂H | O | O | 1 | 2-OMe-Ph |
| A1-1594 | demethylimipramine | N | O | O | CO₂H | O | O | 1 | MeSCH₂ |
| A1-1595 | demethylimipramine | N | O | O | CO₂H | O | O | 1 | MeOCH₂ |
| A1-1596 | demethylimipramine | N | O | O | CO₂H | S | S | 1 | Ph |
| A1-1597 | demethylimipramine | N | O | O | CO₂H | S | 5 | 1 | NMe₂ |
| A1-1598 | demethylimipramine | N | O | O | CO₂H | S | S | 1 | NBn₂ |
| A1-1599 | demethylimipramine | N | O | O | CO₂H | O | S | 1 | Ph |
| A1-1600 | demethylimipramine | N | O | O | CO₂H | O | S | 1 | Me |
| A1-1601 | deprenil | O | O | O | CO₂H | O | O | 1 | methyl |
| A1-1602 | deprenil | O | O | O | CO₂H | O | O | 1 | C(CH₃)₃ |
| A1-1603 | deprenil | O | O | O | CO₂H | O | O | 1 | Ph |
| A1-1604 | deprenil | O | O | O | CO₂H | O | O | 1 | 4-Me-Ph |
| A1-1605 | deprenil | O | O | O | CO₂H | O | O | 1 | 2-pyridyl |
| A1-1606 | deprenil | O | O | O | CO₂H | O | O | 1 | 4-pyridyl |
| A1-1607 | deprenil | O | O | O | CO₂H | O | O | 1 | 2-furyl |
| A1-1608 | deprenil | O | O | O | CO₂H | O | O | 1 | 2-thienyl |
| A1-1609 | deprenil | O | O | O | CO₂H | — | — | 0 | Cl |
| A1-1610 | deprenil | O | O | O | CO₂H | — | — | 0 | {N(Et)₃}⁺Cl⁻ |
| A1-1611 | deprenil | O | O | O | CO₂H | O | O | 1 | CF₃CH₂ |
| A1-1612 | deprenil | O | O | O | CO₂H | O | O | 1 | cyclopropyl |
| A1-1613 | deprenil | O | O | O | CO₂H | O | O | 1 | 2-OMe-Ph |
| A1-1614 | deprenil | O | O | O | CO₂H | O | O | 1 | MeSCH₂ |
| A1-1615 | deprenil | O | O | O | CO₂H | O | O | 1 | MeOCH₂ |
| A1-1616 | deprenil | O | O | O | CO₂H | S | S | 1 | Ph |
| A1-1617 | deprenil | O | O | O | CO₂H | S | S | 1 | NMe₂ |
| A1-1618 | deprenil | O | O | O | CO₂H | S | S | 1 | NBn₂ |
| A1-1619 | deprenil | O | O | O | CO₂H | O | S | 1 | Ph |
| A1-1620 | deprenil | O | O | O | CO₂H | O | S | 1 | Me |
| A1-1621 | doxazosin | O | O | O | CO₂H | O | O | 1 | methyl |
| A1-1622 | doxazosin | O | O | O | CO₂H | O | O | 1 | C(CH₃)₃ |
| A1-1623 | doxazosin | O | O | O | CO₂H | O | O | 1 | Ph |
| A1-1624 | doxazosin | O | O | O | CO₂H | O | O | 1 | 4-Me-Ph |
| A1-1625 | doxazosin | O | O | O | CO₂H | O | O | 1 | 2-pyridyl |
| A1-1626 | doxazosin | O | O | O | CO₂H | O | O | 1 | 4-pyridyl |
| A1-1627 | doxazosin | O | O | O | CO₂H | O | O | 1 | 2-furyl |
| A1-1628 | doxazosin | O | O | O | CO₂H | O | O | 1 | 2-thienyl |
| A1-1629 | doxazosin | O | O | O | CO₂H | — | — | 0 | Cl |
| A1-1630 | doxazosin | O | O | O | CO₂H | — | — | 0 | {N(Et)₃}⁺Cl⁻ |
| A1-1631 | doxazosin | O | O | O | CO₂H | O | O | 1 | CF₃CH₂ |
| A1-1632 | doxazosin | O | O | O | CO₂H | O | O | 1 | cyclopropyl |
| A1-1633 | doxazosin | O | O | O | CO₂H | O | O | 1 | 2-OMe-Ph |
| A1-1634 | doxazosin | O | O | O | CO₂H | O | O | 1 | MeSCH₂ |
| A1-1635 | doxazosin | O | O | O | CO₂H | O | O | 1 | MeOCH₂ |
| A1-1636 | doxazosin | O | O | O | CO₂H | S | S | 1 | Ph |
| A1-1637 | doxazosin | O | O | O | CO₂H | S | S | 1 | NMe₂ |
| A1-1638 | doxazosin | O | O | O | CO₂H | S | S | 1 | NBn₂ |
| A1-1639 | doxazosin | O | O | O | CO₂H | O | S | 1 | Ph |
| A1-1640 | doxazosin | O | O | O | CO₂H | O | S | 1 | Me |
| A1-1641 | enalapril | O | O | O | CO₂H | O | O | 1 | methyl |
| A1-1642 | enalapril | O | O | O | CO₂H | O | O | 1 | C(CH₃)₃ |
| A1-1643 | enalapril | O | O | O | CO₂H | O | O | 1 | Ph |
| A1-1644 | enalapril | O | O | O | CO₂H | O | O | 1 | 4-Me-Ph |
| A1-1645 | enalapril | O | O | O | CO₂H | O | O | 1 | 2-pyridyl |
| A1-1646 | enalapril | O | O | O | CO₂H | O | O | 1 | 4-pyridyl |
| A1-1647 | enalapril | O | O | O | CO₂H | O | O | 1 | 2-furyl |
| A1-1648 | enalapril | O | O | O | CO₂H | O | O | 1 | 2-thienyl |
| A1-1649 | enalapril | O | O | O | CO₂H | — | — | 0 | Cl |
| A1-1650 | enalapril | O | O | O | CO₂H | — | — | 0 | {N(Et)₃}⁺Cl⁻ |
| A1-1651 | enalapril | O | O | O | CO₂H | O | O | 1 | CF₃CH₂ |
| A1-1652 | enalapril | O | O | O | CO₂H | O | O | 1 | cyclopropyl |
| A1-1653 | enalapril | O | O | O | CO₂H | O | O | 1 | 2-OMe-Ph |
| A1-1654 | enalapril | O | O | O | CO₂H | O | O | 1 | MeSCH₂ |
| A1-1655 | enalapril | O | O | O | CO₂H | O | O | 1 | MeOCH₂ |
| A1-1656 | enalapril | O | O | O | CO₂H | S | S | 1 | Ph |
| A1-1657 | enalapril | O | O | O | CO₂H | S | S | 1 | NMe₂ |
| A1-1658 | enalapril | O | O | O | CO₂H | S | S | 1 | NBn₂ |
| A1-1659 | enalapril | O | O | O | CO₂H | O | S | 1 | Ph |
| A1-1660 | enalapril | O | O | O | CO₂H | O | S | 1 | Me |
| A1-1661 | famciclovir | O | O | O | CO₂H | O | O | 1 | methyl |
| A1-1662 | famciclovir | O | O | O | CO₂H | O | O | 1 | C(CH₃)₃ |
| A1-1663 | famciclovir | O | O | O | CO₂H | O | O | 1 | Ph |
| A1-1664 | famciclovir | O | O | O | CO₂H | O | O | 1 | 4-Me-Ph |
| A1-1665 | famciclovir | O | O | O | CO₂H | O | O | 1 | 2-pyridyl |
| A1-1666 | famciclovir | O | O | O | CO₂H | O | O | 1 | 4-pyridyl |
| A1-1667 | famciclovir | O | O | O | CO₂H | O | O | 1 | 2-furyl |
| A1-1668 | famciclovir | O | O | O | CO₂H | O | O | 1 | 2-thienyl |
| A1-1669 | famciclovir | O | O | O | CO₂H | — | — | 0 | Cl |
| A1-1670 | famciclovir | O | O | O | CO₂H | — | — | 0 | {N(Et)₃}⁺Cl⁻ |
| A1-1671 | famciclovir | O | O | O | CO₂H | O | O | 1 | CF₃CH₂ |
| A1-1672 | famciclovir | O | O | O | CO₂H | O | O | 1 | cyclopropyl |
| A1-1673 | famciclovir | O | O | O | CO₂H | O | O | 1 | 2-OMe-Ph |
| A1-1674 | famciclovir | O | O | O | CO₂H | O | O | 1 | MeSCH₂ |
| A1-1675 | famciclovir | O | O | O | CO₂H | O | O | 1 | MeOCH₂ |
| A1-1676 | famciclovir | O | O | O | CO₂H | S | S | 1 | Ph |
| AI-1677 | famciclovir | O | O | O | CO₂H | S | S | 1 | NMe₂ |
| A1-1678 | famciclovir | O | O | O | CO₂H | S | S | 1 | NBn₂ |
| A1-1679 | famciclovir | O | O | O | CO₂H | O | S | 1 | Ph |
| A1-1680 | famciclovir | O | O | O | CO₂H | O | S | 1 | Me |
| AI-1681 | fluoxetine HCl | N | O | O | CO₂H | O | O | 1 | methyl |
| A1-1682 | fluoxetine HCl | N | O | O | CO₂H | O | O | 1 | C(CH₃)₃ |
| A1-1683 | fluoxetine HCl | N | O | O | CO₂H | O | O | 1 | Ph |
| A1-1684 | fluoxetine HCl | N | O | O | CO₂H | O | O | 1 | 4-Me-Ph |
| A1-1685 | fluoxetine HCl | N | O | O | CO₂H | O | O | 1 | 2-pyridyl |
| A1-1686 | fluoxetine HCl | N | O | O | CO₂H | O | O | 1 | 4-pyridyl |
| A1-1687 | fluoxetine HCl | N | O | O | CO₂H | O | O | 1 | 2-furyl |
| A1-1688 | fluoxetine HCl | N | O | O | CO₂H | O | O | 1 | 2-thienyl |
| A1-1689 | fluoxetine HCl | N | O | O | CO₂H | — | — | 0 | Cl |
| A1-1690 | fluoxetine HCl | N | O | O | CO₂H | — | — | 0 | {N(Et)₃}⁺Cl⁻ |
| A1-1691 | fluoxetine HCl | N | O | O | CO₂H | O | O | 1 | CF₃CH₂ |
| A1-1692 | fluoxetine HCl | N | O | O | CO₂H | O | O | 1 | cyclopropyl |
| A1-1693 | fluoxetine HCl | N | O | O | CO₂H | O | O | 1 | 2-OMe-Ph |
| A1-1694 | fluoxetine HCl | N | O | O | CO₂H | O | O | 1 | MeSCH₂ |
| A1-1695 | fluoxetine HCl | N | O | O | CO₂H | O | O | 1 | MeOCH₂ |
| A1-1696 | fluoxetine HCl | N | O | O | CO₂H | S | S | 1 | Ph |
| A1-1697 | fluoxetine HCl | N | O | O | CO₂H | S | S | 1 | NMe₂ |
| A1-1698 | fluoxetine HCl | N | O | O | CO₂H | S | S | 1 | NBn₂ |
| A1-1699 | fluoxetine HCl | N | O | O | CO₂H | O | S | 1 | Ph |
| A1-1700 | fluoxetine HCl | N | O | O | CO₂H | O | S | 1 | Me |
| A1-1701 | gabapentin | N | O | O | CO₂H | O | O | 1 | methyl |
| A1-1702 | gabapentin | N | O | O | CO₂H | O | O | 1 | C(CH₃)a |
| A1-1703 | gabapentin | N | O | O | CO₂H | O | O | 1 | Ph |
| A1-1704 | gabapentin | N | O | O | CO₂H | O | O | 1 | 4-Me-Ph |
| A1-1705 | gabapentin | N | O | O | CO₂H | O | O | 1 | 2-pyridyl |
| A1-1706 | gabapentin | N | O | O | CO₂H | O | O | 1 | 4-pyridyl |
| A1-1707 | gabapentin | N | O | O | CO₂H | O | O | 1 | 2-furyl |
| AJ-1708 | gabapentin | N | O | O | CO₂H | O | O | 1 | 2-thienyl |
| A1-1709 | gabapentin | N | O | O | CO₂H | — | — | 0 | Cl |
| A1-1710 | gabapentin | N | O | O | CO₂H | — | — | 0 | {N(Et)₃}⁺Cl⁻ |
| A1-1711 | gabapentin | N | O | O | CO₂H | O | O | 1 | CF₃CH₂ |
| A1-1712 | gabapentin | N | O | O | CO₂H | O | O | 1 | cyclopropyl |
| A1-1713 | gabapentin | N | O | O | CO₂H | O | O | 1 | 2-OMe-Ph |
| A1-1714 | gabapentin | N | O | O | CO₂H | O | O | 1 | MeSCH₂ |
| A1-1715 | gabapentin | N | O | O | CO₂H | O | O | 1 | MeOCH₂ |
| A1-1716 | gabapentin | N | O | O | CO₂H | S | S | 1 | Ph |
| A1-1717 | gabapentin | N | O | O | CO₂H | S | S | 1 | NMe₂ |
| A1-1718 | gabapentin | N | O | O | CO₂H | S | S | 1 | NBn₂ |
| A1-1719 | gabapentin | N | O | O | CO₂H | O | S | 1 | Ph |
| A1-1720 | gabapentin | N | O | O | CO₂H | O | S | 1 | Me |
| A1-1721 | methylphenidate | N | O | O | CO₂H | O | O | 1 | methyl |
| A1-1722 | methylphenidate | N | O | O | CO₂H | O | O | 1 | C(CH₃)₃ |
| A1-1723 | methylphenidate | N | O | O | CO₂H | O | O | 1 | Ph |
| A1-1724 | methylphenidate | N | O | O | CO₂H | O | O | 1 | 4-Me-Ph |
| A1-1725 | methylphenidate | N | O | O | CO₂H | O | O | 1 | 2-pyridyl |
| A1-1726 | methylphenidate | N | O | O | CO₂H | O | O | 1 | 4-pyridyl |
| A1-1727 | methylphenidate | N | O | O | CO₂H | O | O | 1 | 2-furyl |
| A1-1728 | methylphenidate | N | O | O | CO₂H | O | O | 1 | 2-thienyl |
| A1-1729 | methylphenidate | N | O | O | CO₂H | — | — | 0 | Cl |
| A1-1730 | methylphenidate | N | O | O | CO₂H | — | — | 0 | {N(Et)₃}⁺Cl⁻ |
| A1-1731 | methylphenidate | N | O | O | CO₂H | O | O | 1 | CF₃CH₂ |
| A1-1732 | methylphenidate | N | O | O | CO₂H | O | O | 1 | cyclopropyl |
| A1-1733 | methylphenidate | N | O | O | CO₂H | O | O | 1 | 2-OMe-Ph |
| A1-1734 | methylphenidate | N | O | O | CO₂H | O | O | 1 | MeSCH₂ |
| A1-1735 | methylphenidate | N | O | O | CO₂H | O | O | 1 | MeOCH₂ |
| A1-1736 | methylphenidate | N | O | O | CO₂H | S | S | 1 | Ph |
| A1-1737 | methylphenidate | N | O | O | CO₂H | S | S | 1 | NMe₂ |
| A1-1738 | methylphenidate | N | O | O | CO₂H | S | S | 1 | NBn₂ |
| A1-1739 | methylphenidate | N | O | O | CO₂H | O | S | 1 | Ph |
| A1-1740 | methylphenidate | N | O | O | CO₂H | O | S | 1 | Me |
| A1-1741 | olanzapine | N | O | O | CO₂H | O | O | 1 | methyl |
| A1-1742 | olanzapine | N | O | O | CO₂H | O | O | 1 | C(CH₃)₃ |
| A1-1743 | olanzapine | N | O | O | CO₂H | O | O | 1 | Ph |
| A1-1744 | olanzapine | N | O | O | CO₂H | O | O | 1 | 4-Me-Ph |
| A1-1745 | olanzapine | N | O | O | CO₂H | O | O | 1 | 2-pyridyl |
| A1-1746 | olanzapine | N | O | O | CO₂H | O | O | 1 | 4-pyridyl |
| A1-1747 | olanzapine | N | O | O | CO₂H | O | O | 1 | 2-furyl |
| A1-1748 | olanzapine | N | O | O | CO₂H | O | O | 1 | 2-thienyl |
| A1-1749 | alanzapine | N | O | O | CO₂H | — | — | 0 | Cl |
| A1-1750 | olanzapine | N | O | O | CO₂H | — | — | 0 | {N(Et)₃}⁺Cl⁻ |
| A1-1751 | olanzapine | N | O | O | CO₂H | O | O | 1 | CF₃CH₂ |
| A1-1752 | olanzapine | N | O | O | CO₂H | O | O | 1 | cyclopropyl |
| A1-1753 | olanzapine | N | O | O | CO₂H | O | O | 1 | 2-OMe-Ph |
| A1-1754 | olanzapine | N | O | O | CO₂H | O | O | 1 | MeSCH₂ |
| A1-1755 | olanzapine | N | O | O | CO₂H | O | O | 1 | MeOCH₂ |
| A1-1756 | olanzapine | N | O | O | CO₂H | S | S | 1 | Ph |
| A1-1757 | olanzapine | N | O | O | CO₂H | S | S | 1 | NMe₂ |
| A1-1758 | olanzapine | N | O | O | CO₂H | S | S | 1 | NBn₂ |
| A1-1759 | olanzapine | N | O | O | CO₂H | O | S | 1 | Ph |
| A1-1760 | olanzapine | N | O | O | CO₂H | O | S | 1 | Me |
| A1-1761 | lansoprazole | N | O | O | CO₂H | O | O | 1 | methyl |
| A1-1762 | lansoprazole | N | O | O | CO₂H | O | O | 1 | C(CH₃)₃ |
| A1-1763 | lansoprazole | N | O | O | CO₂H | O | O | 1 | Ph |
| A1-1764 | lansoprazole | N | O | O | CO₂H | O | O | 1 | 4-Me-Ph |
| A1-l765 | lansoprazole | N | O | O | CO₂H | O | O | 1 | 2-pyridyl |
| A1-1766 | lansoprazole | N | O | O | CO₂H | O | O | 1 | 4-pyridyl |
| A1-1767 | lansoprazole | N | O | O | CO₂H | O | O | 1 | 2-furyl |
| A1-1768 | lansoprazole | N | O | O | CO₂H | O | O | 1 | 2-thienyl |
| A1-1769 | lansoprazole | N | O | O | CO₂H | — | — | 0 | Cl |
| A1-1770 | lansoprazole | N | O | O | CO₂H | — | — | 0 | {N(Et)₃}⁺Cl⁻ |
| A1-1771 | lansoprazole | N | O | O | CO₂H | O | O | 1 | CF₃CH₂ |
| A1-1772 | lansoprazole | N | O | O | CO₂H | O | O | 1 | cyclopropyl |
| A1-1773 | lansoprazole | N | O | O | CO₂H | O | O | 1 | 2-OMe-Ph |
| A1-1774 | lansoprazole | N | O | O | CO₂H | O | O | 1 | MeSCH₂ |
| A1-1775 | lansoprazole | N | O | O | CO₂H | O | O | 1 | MeOCH₂ |
| A1-1776 | lansoprazole | N | O | O | CO₂H | S | S | 1 | Ph |
| A1-1777 | lansoprazole | N | O | O | CO₂H | S | S | 1 | NMe₂ |
| A1-1778 | lansoprazole | N | O | O | CO₂H | S | S | 1 | NBn₂ |
| A1-1779 | lansoprazole | N | O | O | CO₂H | O | S | 1 | Ph |
| A1-1780 | lansoprazole | N | O | O | CO₂H | O | S | 1 | Me |
| A1-1781 | omeprazole | N | O | O | CO₂H | O | O | 1 | methyl |
| A1-1782 | omeprazole | N | O | O | CO₂H | O | O | 1 | C(CH₃)₃ |
| A1-1783 | omeprazole | N | O | O | CO₂H | O | O | 1 | Ph |
| A1-1784 | omeprazole | N | O | O | CO₂H | O | O | 1 | 4-Me-Ph |
| A1-1785 | omeprazole | N | O | O | CO₂H | O | O | 1 | 2-pyridyl |
| A1-1786 | omeprazole | N | O | O | CO₂H | O | O | 1 | 4-pyridyl |
| A1-1787 | omeprazole | N | O | O | CO₂H | O | O | 1 | 2-furyl |
| A1-1788 | omeprazole | N | O | O | CO₂H | O | O | 1 | 2-thienyl |
| A1-1789 | omeprazole | N | O | O | CO₂H | — | — | 0 | Cl |
| A1-1790 | omeprazole | N | O | O | CO₂H | — | — | 0 | {N(Et)₃}⁺Cl⁻ |
| A1-1791 | omeprazole | N | O | O | CO₂H | O | O | 1 | CF₃CH₂ |
| A1-1792 | omeprazole | N | O | O | CO₂H | O | O | 1 | cyclopropyl |
| A1-1793 | omeprazole | N | O | O | CO₂H | O | O | 1 | 2-OMe-Ph |
| A1-1794 | omeprazole | N | O | O | CO₂H | O | O | 1 | MeSCH₂ |
| A1-1795 | omeprazole | N | O | O | CO₂H | O | O | 1 | MeOCH₂ |
| A1-1796 | omeprazole | N | O | O | CO₂H | S | S | 1 | Ph |
| A1-1797 | omeprazole | N | O | O | CO₂H | S | S | 1 | NMe₂ |
| A1-1798 | omeprazole | N | O | O | CO₂H | S | S | 1 | NBn₂ |
| A1-1799 | omeprazole | N | O | O | CO₂H | O | S | 1 | Ph |
| A1-1800 | omeprazole | N | O | O | CO₂H | O | S | 1 | Me |
| A1-1801 | omeprazole | O | O | O | CO₂H | O | O | 1 | methyl |
| A1-1802 | omeprazole | O | O | O | CO₂H | O | O | 1 | C(CH₃)₃ |
| A1-1803 | omeprazole | O | O | O | CO₂H | O | O | 1 | Ph |
| A1-1804 | omeprazole | O | O | O | CO₂H | O | O | 1 | 4-Me-Ph |
| A1-1805 | omeprazole | O | O | O | CO₂H | O | O | 1 | 2-pyridyl |
| A1-1806 | omeprazole | O | O | O | CO₂H | O | O | 1 | 4-pyridyl |
| A1-1807 | omeprazole | O | O | O | CO₂H | O | O | 1 | 2-furyl |
| A1-1808 | omeprazole | O | O | O | CO₂H | O | O | 1 | 2-thienyl |
| A1-1809 | omeprazole | O | O | O | CO₂H | — | — | 0 | Cl |
| A1-1810 | omeprazole | O | O | O | CO₂H | — | — | 0 | {N(Et)₃}⁺Cl⁻ |
| A1-1811 | omeprazole | O | O | O | CO₂H | O | O | 1 | CF₃CH₂ |
| A1-1812 | omeprazole | O | O | O | CO₂H | O | O | 1 | cyclopropyl |
| A1-1813 | omeprazole | O | O | O | CO₂H | O | O | 1 | 2-OMe-Ph |
| A1-1814 | omeprazole | O | O | O | CO₂H | O | O | 1 | MeSOH₂ |
| A1-1815 | omeprazole | O | O | O | CO₂H | O | O | 1 | MeOCH₂ |
| A1-1816 | omeprazole | O | O | O | CO₂H | S | S | 1 | Ph |
| A1-1817 | omeprazole | O | O | O | CO₂H | S | S | 1 | NMe₂ |
| A1-1818 | omeprazole | O | O | O | CO₂H | S | S | 1 | NBn₂ |
| A1-1819 | omeprazole | O | O | O | CO₂H | O | S | 1 | Ph |
| A1-1820 | omeprazole | O | O | O | CO₂H | O | S | 1 | Me |
| A1-1821 | phentermine | O | O | O | CO₂H | O | O | 1 | methyl |
| A1-1822 | phentermine | O | O | O | CO₂H | O | O | 1 | C(CH₃)₃ |
| A1-1823 | phentermine | O | O | O | CO₂H | O | O | 1 | Ph |
| A1-1824 | phentermine | O | O | O | CO₂H | O | O | 1 | 4-Me-Ph |
| A1-1825 | phentermine | O | O | O | CO₂H | O | O | 1 | 2-pyridyl |
| A1-1826 | phentermine | O | O | O | CO₂H | O | O | 1 | 4-pyridyl |
| A1-1827 | phentermine | O | O | O | CO₂H | O | O | 1 | 2-furyl |
| A1-1828 | phentermine | O | O | O | CO₂H | O | O | 1 | 2-thienyl |
| A1-1829 | phentermine | O | O | O | CO₂H | — | — | 0 | Cl |
| A1-1830 | phentermine | O | O | O | CO₂H | — | — | 0 | {N(Et)₃}⁺Cl⁻ |
| A1-1831 | phentermine | O | O | O | CO₂H | O | O | 1 | CF₃CH₂ |
| A1-1832 | phentermine | O | O | O | CO₂H | O | O | 1 | cyclopropyl |
| A1-1833 | phentermine | O | O | O | CO₂H | O | O | 1 | 2-OMe-Ph |
| A1-1834 | phentermine | O | O | O | CO₂H | O | O | 1 | MeSCH₂ |
| A1-1835 | phentermine | O | O | O | CO₂H | O | O | 1 | MeOCH₂ |
| A1-1836 | phentermine | O | O | O | CO₂H | S | S | 1 | Ph |
| A1-1837 | phentermine | O | O | O | CO₂H | S | S | 1 | NMe₂ |
| A1-1838 | phentermine | O | O | O | CO₂H | S | S | 1 | NBn₂ |
| A1-1839 | phentermine | O | O | O | CO₂H | O | S | 1 | Ph |
| A1-1840 | phentermine | O | O | O | CO₂H | O | S | 1 | Me |
| A1-1841 | paroxetine | N | O | O | CO₂H | O | O | 1 | methyl |
| A1-1842 | paroxetine | N | O | O | CO₂H | O | O | 1 | C(CH₃)a |
| A1-1843 | paroxetine | N | O | O | CO₂H | O | O | 1 | Ph |
| A1-1844 | paroxetine | N | O | O | CO₂H | O | O | 1 | 4-Me-Ph |
| A1-1845 | paroxetine | N | O | O | CO₂H | O | O | 1 | 2-pyridyl |
| A1-1846 | paroxetine | N | O | O | CO₂H | O | O | 1 | 4-pyridyl |
| A1-1847 | paroxetine | N | O | O | CO₂H | O | O | 1 | 2-furyl |
| A1-1848 | paroxetine | N | O | O | CO₂H | O | O | 1 | 2-thienyl |
| A1-1849 | paroxetine | N | O | O | CO₂H | — | — | 0 | Cl |
| A1-1850 | paroxetine | N | O | O | CO₂H | — | — | 0 | {N(Et)₃}⁺Cl⁻ |
| A1-1851 | paroxetine | N | O | O | CO₂H | O | O | 1 | CF₃CH₂ |
| A1-1852 | paroxetine | N | O | O | CO₂H | O | O | 1 | cyclopropyl |
| A1-1853 | paroxetine | N | O | O | CO₂H | O | O | 1 | 2-OMe-Ph |
| A1-1854 | paroxetine | N | O | O | CO₂H | O | O | 1 | MeSCH₂ |
| A1-1855 | paroxetine | N | O | O | CO₂H | O | O | 1 | MeOCH₂ |
| A1-1856 | paroxetine | N | O | O | CO₂H | S | S | 1 | Ph |
| A1-1857 | paroxetine | N | O | O | CO₂H | S | S | 1 | NMe₂ |
| A1-1858 | paroxetine | N | O | O | CO₂H | S | S | 1 | NBn₂ |
| A1-1859 | paroxetine | N | O | O | CO₂H | O | S | 1 | Ph |
| A1-1860 | paroxetine | N | O | O | CO₂H | O | S | 1 | Me |
| A1-1861 | quinapril | N | O | O | CO₂H | O | O | 1 | methyl |
| A1-1862 | quinapril | N | O | O | CO₂H | O | O | 1 | C(CH₃)₃ |
| A1-1863 | quinapril | N | O | O | CO₂H | O | O | 1 | Ph |
| A1-1864 | quinapril | N | O | O | CO₂H | O | O | 1 | 4-Me-Ph |
| A1-1865 | quinapril | N | O | O | CO₂H | O | O | 1 | 2-pyridyl |
| A1-1866 | quinapril | N | O | O | CO₂H | O | O | 1 | 4-pyridyl |
| A1-1867 | quinapril | N | O | O | CO₂H | O | O | 1 | 2-furyl |
| A1-1868 | quinapril | N | O | O | CO₂H | O | O | 1 | 2-thienyl |
| A1-1869 | quinapril | N | O | O | CO₂H | — | — | 0 | Cl |
| A1-1870 | quinapril | N | O | O | CO₂H | — | — | 0 | {N(Et)₃}⁺Cl⁻ |
| A1-1871 | quinapril | N | O | O | CO₂H | O | O | 1 | CF₃CH₂ |
| A1-1872 | quinapril | N | O | O | CO₂H | O | O | 1 | cyclopropyl |
| A1-1873 | quinapril | N | O | O | CO₂H | O | O | 1 | 2-OMe-Ph |
| A1-1874 | quinapril | N | O | O | CO₂H | O | O | 1 | MeSCH₂ |
| A1-1875 | quinapril | N | O | O | CO₂H | O | O | 1 | MeOCH₂ |
| A1-1876 | quinapril | N | O | O | CO₂H | S | S | 1 | Ph |
| A1-1877 | quinapril | N | O | O | CO₂H | S | S | 1 | NMe₂ |
| A1-1878 | quinapril | N | O | O | CO₂H | S | S | 1 | NBn₂ |
| A1-1879 | quinapril | N | O | O | CO₂H | O | S | 1 | Ph |
| A1-1880 | quinapril | N | O | O | CO₂H | O | S | 1 | Me |
| A1-1881 | sertraline | N | O | O | CO₂H | O | O | 1 | methyl |
| A1-1882 | sertraline | N | O | O | CO₂H | O | O | 1 | C(CH₃)a |
| A1-1883 | sertraline | N | O | O | CO₂H | O | O | 1 | Ph |
| A1-1884 | sertraline | N | O | O | CO₂H | O | O | 1 | 4-Me-Ph |
| A1-1885 | sertraline | N | O | O | CO₂H | O | O | 1 | 2-pyridyl |
| A1-1886 | sertraline | N | O | O | CO₂H | O | O | 1 | 4-pyridyl |
| A1-1887 | sertraline | N | O | O | CO₂H | O | O | 1 | 2-furyl |
| A1-1888 | sertraline | N | O | O | CO₂H | O | O | 1 | 2-thienyl |
| A1-1889 | sertraline | N | O | O | CO₂H | — | — | 0 | Cl |
| A1-1890 | sertraline | N | O | O | CO₂H | — | — | 0 | {N(Et)₃}⁺Cl⁻ |
| A1-1891 | sertraline | N | O | O | CO₂H | O | O | 1 | CF₃CH₂ |
| A1-1892 | sertraline | N | O | O | CO₂H | O | O | 1 | cyclopropyl |
| A1-1893 | sertraline | N | O | O | CO₂H | O | O | 1 | 2-OMe-Pli |
| A1-1894 | sertraline | N | O | O | CO₂H | O | O | 1 | MeSCH₂ |
| A1-1895 | sertraline | N | O | O | CO₂H | O | O | 1 | MeOCH₂ |
| A1-1896 | sertraline | N | O | O | CO₂H | S | S | 1 | Ph |
| A1-1897 | sertraline | N | O | O | CO₂H | S | S | 1 | NMe₂ |
| A1-1898 | sertraline | N | O | O | CO₂H | S | S | 1 | NBn₂ |
| A1-1899 | sertraline | N | O | O | CO₂H | O | S | 1 | Ph |
| A1-1900 | sertraline | N | O | O | CO₂H | O | S | 1 | Me |
| A1-1901 | bupropion | N | O | O | CO₂H | O | O | 1 | methyl |
| A1-1902 | bupropion | N | O | O | CO₂H | O | O | 1 | C(CH₃)a |
| A1-1903 | bupropion | N | O | O | CO₂H | O | O | 1 | Ph |
| A1-l904 | bupropion | N | O | O | CO₂H | O | O | 1 | 4-Me-Ph |
| A1-l905 | bupropion | N | O | O | CO₂H | O | O | 1 | 2-pyridyl |
| A1-1906 | bupropion | N | O | O | CO₂H | O | O | 1 | 4-pyridyl |
| A1-1907 | bupropion | N | O | O | CO₂H | O | O | 1 | 2-furyl |
| A1-1908 | bupropion | N | O | O | CO₂H | O | O | 1 | 2-thienyl |
| A1-1909 | bupropion | N | O | O | CO₂H | — | — | 0 | Cl |
| A1-1910 | bupropion | N | O | O | CO₂H | — | — | 0 | {N(Et)₃}⁺Cl⁻ |
| A1-1911 | bupropion | N | O | O | CO₂H | O | O | 1 | CF₃CH₂ |
| A1-1912 | bupropion | N | O | O | CO₂H | O | O | 1 | cyclopropyl |
| A1-1913 | bupropion | N | O | O | CO₂H | O | O | 1 | 2-OMe-Ph |
| A1-1914 | bupropion | N | O | O | CO₂H | O | O | 1 | MeSCH₂ |
| A1-1915 | bupropion | N | O | O | CO₂H | O | O | 1 | MeOCH₂ |
| A1-1916 | bupropion | N | O | O | CO₂H | S | S | 1 | Ph |
| A1-1917 | bupropion | N | O | O | CO₂H | S | S | 1 | NMe₂ |
| A1-1918 | bupropion | N | O | O | CO₂H | S | S | 1 | NBn₂ |
| A1-1919 | bupropion | N | O | O | CO₂H | O | S | 1 | Ph |
| A1-1920 | bupropion | N | O | O | CO₂H | O | S | 1 | Me |

Table A2: Compounds (A2-1)-(A2-1920) are compounds of Formula I where X¹, Z(X¹)ₘ—H, G¹⁰, G¹¹, R², G²⁰, G²¹, m, t, q and Z² are identical to those in Table A1 except for R¹ which equals CO₂Me.

Table A3: Compounds (A3-1)-(A3-1920) are compounds of Formula I where X¹, Z¹(X¹)ₘ—H, G¹⁰, G¹¹, R², G²⁰, G²1, m, t, q and Z² are identical to those in Table A1 except for R¹ which equals CO₂Et.

Table A4: Compounds (A4-1)-(A4-1920) are compounds of Formula I where X¹, Z¹(X¹)ₘ—H, G¹⁰, G¹¹, R², G²⁰, G²¹, m, t, q and Z² are identical to those in Table A1 except for R¹ which equals Co₂n-Pr.

Table A5: Compounds (A5-1)-(A5-1920) are compounds of Formula I where X¹, Z¹(X¹)ₘ—H, G¹⁰, G¹¹, R², G²⁰, G²1, m, t, q and Z² are identical to those in Table A1 except for R¹ which equals CO₂t-Bu.

Table A6: Compounds (A6-1)-(A6-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂cyclopropyl.

Table A7: Compounds (A7-1)-(A7-1920) are compounds of Formula I where X¹, Z¹(X¹)ₘ—H, G¹⁰, G¹¹, R², G²⁰, G²¹, m, t, q and Z² are identical to those in Table A1 except for R¹ which equals Co₂Bn.

Table A8: Compounds (A8-1)-(A8-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂CH₂CH₂Ph.

Table A9: Compounds (A9-1)-(A9-1920) are compounds of Formula I where X¹, Z¹(X¹)-H, G¹⁰, G¹¹, R², G²⁰, G²¹, m, t, q and Z² are identical to those in Table A1 except for R¹ which equals CO₂CH₂CF₃.

Table A10: Compounds (A10-1)-(A10-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂CH₂OMe.

Table A11: Compounds (A11-1)-(A11-1920) are compounds of Formula I where X¹, Z¹(X¹), —H, G¹⁰, G¹¹, R², G²⁰, G²¹, m, t, q and Z² are identical to those in Table A1 except for R¹ which equals Co₂allyl.

Table A12: Compounds (A12-1)-(A12-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂Ph.

Table A13: Compounds (A13-1)-(A13-1920) are compounds of Formula I where X¹, Z¹(X¹)ₘ—H, G¹⁰, G¹¹, R², G²⁰, G²¹, m, t, q and Z² are identical to those in Table A1 except for R¹ which equals Co₂-2(OMe)Ph.

Table A14: Compounds (A14-1)-(A14-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂-3-(OMe)Ph.

Table A15: Compounds (A15-1)-(A15-1920) are compounds of Formula I where X¹, Z¹(X¹), —H, G¹⁰, G¹¹, R², G²⁰, G²¹, m, t, q and Z² are identical to those in Table A1 except for R¹ which equals CO₂-4-(OMe)-Ph.

Table A16: Compounds (A16-1)-(A16-1920) are compounds of Formula I where X¹, Z¹(X¹)ₘ—H, G¹⁰, G¹¹, R², G²⁰, G²¹, m, t, q and Z² are identical to those in Table A1 except for R¹ which equals Co₂-2(Me)Ph.

Table A17: Compounds (A17-1)-(A17-1920) are compounds of Formula I where X¹, Z¹(X¹)ₘ—H, G¹⁰, G¹¹, R², G²⁰, G²¹, m, t, q and Z² are identical to those in Table A1 except for R¹ which equals Co₂-3(Me)Ph.

Table A18: Compounds (A18-1)-(A18-1920) are compounds of Formula I where X¹, Z¹(X)ₘ-H, G¹⁰, G¹¹, R², G²⁰, G²¹, m, t, q and Z² are identical to those in Table A1 except for R¹ which equals Co₂-4(Me)Ph.

Table A19: Compounds (A19-1)-(A19-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂-4(NO₂)Ph.

Table A20: Compounds (A20-1)-(A20-1920) are compounds of Formula I where X¹, Z¹(X¹)ₘ—H, G¹⁰, G¹¹, R², G²⁰, G²¹, m, t, q and Z² are identical to those in Table A1 except for R¹ which equals Co₂SiMe₃.

Table A21: Compounds (A21-1)-(A21-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)Me.

Table A22: Compounds (A22-1)-(A22-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)NMe₂.

Table A23: Compounds (A23-1)-(A23-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)NBn₂.

Table A24: Compounds (A24-1)-(A24-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(Me)Bn.

Table A25: Compounds (A25-1)-(A25-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(Me)Ph.

Table A26: Compounds (A26-1)-(A26-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)Ph.

Table A27: Compounds (A27-1)-(A27-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)NMe₂.

Table A28: Compounds (A28-1)-(A28-1920) are compounds of Formula I where X¹, Z¹(X¹)ₘ—H, G¹⁰, G¹¹, R², G²⁰, G²¹, m, t, q and Z² are identical to those in Table A1 except for R¹ which equals CO₂n-octyl.

Table A29: Compounds (A29-1)-(A29-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)ₙ-Bu.

Table A30: Compounds (A30-1)-(A30-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)N(H)CO₂Et.

Table A31: Compounds (A31-1)-(A31-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)N(H)Co₂t-Bu.

Table A32: Compounds (A32-1)-(A32-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)N═CHMe.

Table A33: Compounds (A33-1)-(A33-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)N═CMe₂.

Table A34: Compounds (A34-1)-(A34-1920) are compounds of Formula I where X¹, Z¹(X¹)ₘ—H, G¹⁰, G¹¹, R², G²⁰, G²¹, m, t, q and Z² are identical to those in Table AI except for R¹ which equals C(═O)N(H)N═CHPh.

Table A35: Compounds (A35-1)-(A35-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)SPh.

Table A36: Compounds (A36-1)-(A36-1920) are compounds of Formula I where X¹, Z¹(X¹)ₘ—H, G¹⁰, G¹¹, R², G²⁰, G²′, m, t, q and Z² are identical to those in Table A1 except for R¹ which equals C(═O)SEt.

Table A37: Compounds (A37-1)-(A37-1920) are compounds of Formula I where X¹, Z¹(X¹)ₘ—H, G¹⁰, G¹¹, R², G²⁰, G²′, m, t, q and Z² are identical to those in Table A1 except for R¹ which equals C(═O)Sn-Bu.

Table A38: Compounds (A38-1)-(A38-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)OMe.

Table A39: Compounds (A39-1)-(A39-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)OBn.

Table A40: Compounds (A40-1)-(A40-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)NH₂.

Table A41: Compounds (A41-1)-(A41-1920) are compounds of Formula I where X¹, Z¹(X¹)ₘ—H, G¹⁰, G¹¹, R², G²⁰, G²¹, m, t, q and Z² are identical to those in Table A1 except for R¹ which equals C(═O)N(H)OH.

Table B1 describes additional examples of compounds of Formula I which can be made using the procedures described hereinbefore, where R² is hydrogen, m=0, q=1, t=0 or 1 and the pharmaceutical which defines the pharmaceutical moiety of these examples is Z²(X²)—[(C=G²⁰)-G²¹]-H, Z²(X²)_{q}—H or Z²(X²). The following groups, Z¹, G¹⁰, G¹¹, R¹, G²⁰, G²¹, t, X² and Z²(X²)—[(C=G²⁰)-G²¹]-H, Z²(X²)_{q}—H or Z²(X²) are defined within Table B1.

TABLE B1

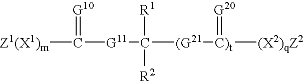

| | | | | | | | | | Z²(X²)_{q}—[(C═G²⁰)— |
|---|---|---|---|---|---|---|---|---|---|
| | | | | | | | | | G²¹]t-H or when t = 0, |
| Cmpd# | Z¹ | G¹⁰ | G¹¹ | R¹ | G²⁰ | G²¹ | t | X² | Z²(X²)_{q} or Z²(X²)_{q}—H |
| B1-1 | Me | O | O | CO₂H | — | — | 0 | N | bisacodyl |
| B1-2 | cyclohexyl | O | O | CO₂H | — | — | 0 | N | bisacodyl |
| B1-3 | t-Bu | O | O | CO₂H | — | — | 0 | N | bisacodyl |
| B1-4 | CF₃CH₂ | O | O | CO₂H | — | — | 0 | N | bisacodyl |
| B1-5 | allyl | O | O | CO₂H | — | — | 0 | N | bisacodyl |
| B1-6 | 4-(NO₂)Ph | O | O | CO₂H | — | — | 0 | N | bisacodyl |
| B1-7 | PhS | O | O | CO₂H | — | — | 0 | N | bisacodyl |
| B1-8 | N(Me)Ph | O | O | CO₂H | — | — | 0 | N | bisacodyl |
| B1-9 | N(H)OMe | O | O | CO₂H | — | — | 0 | N | bisacodyl |
| B1-10 | N(H)CO₂Et | O | O | CO₂H | — | — | 0 | N | bisacodyl |
| B1-11 | Me | O | O | CO₂H | — | — | 0 | N | bupivacaine |
| B1-12 | cyclohexyl | O | O | CO₂H | — | — | 0 | N | bupivacaine |
| B1-13 | t-Bu | O | O | CO₂H | — | — | 0 | N | bupivacaine |
| B1-14 | CF₃CH₂ | O | O | CO₂H | — | — | 0 | N | bupivacaine |
| B1-15 | allyl | O | O | CO₂H | — | — | 0 | N | bupivacaine |
| B1-16 | 4-(NO₂)Ph | O | O | CO₂H | — | — | 0 | N | bupivacaine |
| B1-17 | PhS | O | O | CO₂H | — | — | 0 | N | bupivacaine |
| B1-18 | N(Me)Ph | O | O | CO₂H | — | — | 0 | N | bupivacaine |
| B1-19 | N(H)OMe | O | O | CO₂H | — | — | 0 | N | bupivacaine |
| B1-20 | N(H)CO₂Et | O | O | CO₂H | — | — | 0 | N | bupivacaine |
| B1-21 | Me | O | O | CO₂H | — | — | 0 | N | chloroprocaine |
| B1-22 | cyclohexyl | O | O | CO₂H | — | — | 0 | N | chloroprocaine |
| B1-23 | t-Bu | O | O | CO₂H | — | — | 0 | N | chloroprocaine |
| B1-24 | CF₃CH₂ | O | O | CO₂H | — | — | 0 | N | chloroprocaine |
| B1-25 | allyl | O | O | CO₂H | — | — | 0 | N | chloroprocaine |
| B1-26 | 4-(NO₂)Ph | O | O | CO₂H | — | — | 0 | N | chloroprocaine |
| B1-27 | PhS | O | O | CO₂H | — | — | 0 | N | chloroprocaine |
| B1-28 | N(Me)Ph | O | O | CO₂H | — | — | 0 | N | chloroprocaine |
| B1-29 | N(H)OMe | O | O | CO₂H | — | — | 0 | N | chloroprocaine |
| B1-30 | N(H)CO₂Et | O | O | CO₂H | — | — | 0 | N | chloroprocaine |
| B1-31 | Me | O | O | CO₂H | — | — | 0 | N | tetracaine |
| B1-32 | cyclohexyl | O | O | CO₂H | — | — | 0 | N | tetracaine |
| B1-33 | t-Bu | O | O | CO₂H | — | — | 0 | N | tetracaine |
| B1-34 | CF₃CH₂ | O | O | CO₂H | — | — | 0 | N | tetracaine |
| B1-35 | allyl | O | O | CO₂H | — | — | 0 | N | tetracaine |
| B1-36 | 4-(NO₂)Ph | O | O | CO₂H | — | — | 0 | N | tetracaine |
| B1-37 | PhS | O | O | CO₂H | — | — | 0 | N | tetracaine |
| B1-38 | N(Me)Ph | O | O | CO₂H | — | — | 0 | N | tetracaine |
| B1-39 | N(H)OMe | O | O | CO₂H | — | — | 0 | N | tetracaine |
| B1-40 | N(H)CO₂Et | O | O | CO₂H | — | — | 0 | N | tetracaine |
| B1-41 | Me | O | O | CO₂H | — | — | 0 | N | acrivistine |
| B1-42 | cyclohexyl | O | O | CO₂H | — | — | 0 | N | acrivistine |
| B1-43 | t-Bu | O | O | CO₂H | — | — | 0 | N | acrivistine |
| B1-44 | CF₃CH₂ | O | O | CO₂H | — | — | 0 | N | acrivistine |
| B1-45 | allyl | O | O | CO₂H | — | — | 0 | N | acrivistine |
| B1-46 | 4-(NO₂)Ph | O | O | CO₂H | — | — | 0 | N | acrivistine |
| B1-47 | PhS | O | O | CO₂H | — | — | 0 | N | acrivistine |
| B1-48 | N(Me)Ph | O | O | CO₂H | — | — | 0 | N | acrivistine |
| B1-49 | N(H)OMe | O | O | CO₂H | — | — | 0 | N | acrivistine |
| B1-50 | N(H)CO₂Et | O | O | CO₂H | — | — | 0 | N | acrivistine |
| B1-51 | Me | O | O | CO₂H | — | — | 0 | N | amiodarone |
| B1-52 | cyclohexyl | O | O | CO₂H | — | — | 0 | N | amiodarone |
| B1-53 | t-Bu | O | O | CO₂H | — | — | 0 | N | amiodarone |
| B1-54 | CF₃CH₂ | O | O | CO₂H | — | — | 0 | N | amiodarone |
| B1-55 | allyl | O | O | CO₂H | — | — | 0 | N | amiodarone |
| B1-56 | 4-(NO₂)Ph | O | O | CO₂H | — | — | 0 | N | amiodarone |
| B1-57 | PhS | O | O | CO₂H | — | — | 0 | N | amiodarone |
| B1-58 | N(Me)Ph | O | O | CO₂H | — | — | 0 | N | amiodarone |
| B1-59 | N(H)OMe | O | O | GO2H | — | — | 0 | N | amiodarone |
| B1-60 | N(H)CO₂Et | O | O | CO₂H | — | — | 0 | N | amiodarone |
| B1-61 | Me | O | O | CO₂H | — | — | 0 | N | amitriptyline |
| B1-62 | cyclohexyl | O | O | CO₂H | — | — | 0 | N | amitriptyline |
| B1-63 | t-Bu | O | O | CO₂H | — | — | 0 | N | amitriptyline |
| B1-64 | CF₃CH₂ | O | O | CO₂H | — | — | 0 | N | amitriptyline |
| B1-65 | allyl | O | O | CO₂H | — | — | 0 | N | amitriptylme |
| B1-66 | 4-(NO₂)Ph | O | O | CO₂H | — | — | 0 | N | amitriptyline |
| B1-67 | PhS | O | O | CO₂H | — | — | 0 | N | amitriptyline |
| B1-68 | N(Me)Ph | O | O | CO₂H | — | — | 0 | N | amitriptyline |
| B1-69 | N(H)OMe | O | O | CO₂H | — | — | 0 | N | amitriptyline |
| B1-70 | N(H)CO₂Et | O | O | CO₂H | — | — | 0 | N | amitriptyline |
| B1-71 | Me | O | O | CO₂H | — | — | 0 | N | amrinone |
| B1-72 | cyclohexyl | O | O | CO₂H | — | — | 0 | N | amrinone |
| B1-73 | t-Bu | O | O | CO₂H | — | — | 0 | N | amrinone |
| B1-74 | CF₃CH₂ | O | O | CO₂H | — | — | 0 | N | amrinone |
| B1-75 | allyl | O | O | CO₂H | — | — | 0 | N | amrinone |
| B1-76 | 4-(NO₂)Ph | O | O | CO₂H | — | — | 0 | N | amrinone |
| B1-77 | PhS | O | O | CO₂H | — | — | 0 | N | amrinone |
| B1-78 | N(Me)Ph | O | O | CO₂H | — | — | 0 | N | amrinone |
| B1-79 | N(H)OMe | O | O | CO₂H | — | — | 0 | N | amrinone |
| B1-80 | N(H)CO₂Et | O | O | CO₂H | — | — | 0 | N | amrinone |
| B1-81 | Me | O | O | CO₂H | — | — | 0 | N | atropine |
| B1-82 | cyclohexyl | O | O | CO₂H | — | — | 0 | N | atropine |
| B1-83 | t-Bu | O | O | CO₂H | — | — | 0 | N | atropine |
| B1-84 | CF₃CH₂ | O | O | CO₂H | — | — | 0 | N | atropine |
| B1-85 | allyl | O | O | CO₂H | — | — | 0 | N | atropine |
| B1-86 | 4-(NO₂)Ph | O | O | CO₂H | — | — | 0 | N | atropine |
| B1-87 | PhS | O | O | CO₂H | — | — | 0 | N | atropine |
| BL-88 | N(Me)Ph | O | O | CO₂H | — | — | 0 | N | atropine |
| B1-89 | N(H)OMe | O | O | CO₂H | — | — | 0 | N | atropine |
| B1-90 | N(H)CO₂Et | O | O | CO₂H | — | — | 0 | N | atropine |
| B1-91 | Me | O | O | CO₂H | — | — | 0 | N | benzphetamine |
| B1-92 | cyclohexyl | O | O | 00211 | — | — | 0 | N | benzphetamine |
| B1-93 | t-Bu | O | O | CO₂H | — | — | 0 | N | benzphetamine |
| B1-94 | CF₃CH₂ | O | O | CO₂H | — | — | 0 | N | benzphetamine |
| B1-95 | allyl | O | O | CO₂H | — | — | 0 | N | benzphetamine |
| B1-96 | 4-(NO₂)Ph | O | O | CO₂H | — | — | 0 | N | benzphetamine |
| B1-97 | PhS | O | O | CO₂H | — | — | 0 | N | benzphetamine |
| B1-98 | N(Me)Ph | O | O | CO₂H | — | — | 0 | N | benzphetamine |
| B1-99 | N(H)OMe | O | O | CO₂H | — | — | 0 | N | benzphetamine |
| B1-100 | N(H)CO₂Et | O | O | CO₂H | — | — | 0 | N | benzphetamine |
| B1-101 | Me | O | O | CO₂H | — | — | 0 | N | beperiden |
| B1-102 | cyclohexyl | O | O | CO₂H | — | — | 0 | N | beperiden |
| B1-103 | t-Bu | O | O | CO₂H | — | — | 0 | N | beperiden |
| B1-104 | CF₃CH₂ | O | O | CO₂H | — | — | 0 | N | beperiden |
| B1-105 | allyl | O | O | CO₂H | — | — | 0 | N | beperiden |
| B1-106 | 4-(NO₂)Ph | O | O | CO₂H | — | — | 0 | N | beperiden |
| B1-107 | PhS | O | O | CO₂H | — | — | 0 | N | beperiden |
| B1-108 | N(Me)Ph | O | O | CO₂H | — | — | 0 | N | beperiden |
| B1-109 | N(H)OMe | O | O | CO₂H | — | — | 0 | N | beperiden |
| B1-110 | N(H)CO₂Et | O | O | CO₂H | — | — | 0 | N | beperiden |
| B1-111 | Me | O | O | CO₂H | — | — | 0 | N | bromopheniramine |
| B1-112 | cyclohexyl | O | O | CO₂H | — | — | 0 | N | bromopheniramine |
| B1-113 | t-Bu | O | O | CO₂H | — | — | 0 | N | bromopheniramine |
| B1-114 | CF₃CH₂ | O | O | CO₂H | — | — | 0 | N | bromopheniramine |
| B1-115 | allyl | O | O | CO₂H | — | — | 0 | N | bromopheniramine |
| B1-116 | 4-(NO₂)Ph | O | O | CO₂H | — | — | 0 | N | bromopheniramine |
| B1-117 | PhS | O | O | CO₂H | — | — | 0 | N | bromopheniramine |
| B1-118 | N(Me)Ph | O | O | CO₂H | — | — | 0 | N | bromopheniramine |
| B1-119 | N(H)OMe | O | O | CO₂H | — | — | 0 | N | bromopheniramine |
| B1-120 | N(H)CO₂Et | O | O | CO₂H | — | — | 0 | N | bromopheniramine |
| B1-121 | Me | O | O | CO₂H | — | — | 0 | N | clemastine |
| B1-122 | cyclohexyl | O | O | CO₂H | — | — | 0 | N | clemastine |
| B1-123 | t-Bu | O | O | CO₂H | — | — | 0 | N | clemastine |
| B1-124 | CF₃CH₂ | O | O | CO₂H | — | — | 0 | N | clemastine |
| B1-125 | allyl | O | O | CO₂H | — | — | 0 | N | clemastine |
| B1-126 | 4-(NO₂)Ph | O | O | CO₂H | — | — | 0 | N | clemastine |
| B1-127 | PhS | O | O | CO₂H | — | — | 0 | N | clemastine |
| B1-128 | N(Me)Ph | 0 | 0 | CO₂H | — | — | 0 | N | clemastine |
| B1-129 | N(H)OMe | O | O | CO₂H | — | — | 0 | N | clemastine |
| B1-130 | N(H)CO₂Et | O | O | CO₂H | — | — | 0 | N | clemastine |
| B1-131 | Me | O | O | CO₂H | — | — | 0 | N | clomiphene |
| B1-132 | cyclohexyl | O | O | CO₂H | — | — | 0 | N | clomiphene |
| B1-133 | t-Bu | O | O | CO₂H | — | — | 0 | N | clomiphene |
| B1-134 | CF₃CH₂ | O | O | CO₂H | — | — | 0 | N | clomiphene |
| B1-135 | allyl | O | O | CO₂H | — | — | 0 | N | clomiphene |
| B1-136 | 4-(NO₂)Ph | O | O | CO₂H | — | — | 0 | N | clomiphene |
| B1-137 | PhS | O | O | CO₂H | — | — | 0 | N | clomiphene |
| B1-138 | N(Me)Ph | O | O | CO₂H | — | — | 0 | N | clomiphene |
| B1-139 | N(H)OMe | O | O | CO₂H | — | — | 0 | N | clomiphene |
| B1-140 | N(H)CO₂Et | O | O | CO₂H | — | — | 0 | N | clomiphene |
| B1-141 | Me | O | O | CO₂H | — | — | 0 | N | cyclobenzaprine |
| B1-142 | cyclohexyl | O | O | CO₂H | — | — | 0 | N | cyclobenzaprine |
| B1-143 | t-Bu | O | O | CO₂H | — | — | 0 | N | cyclobenzaprine |
| B1-144 | CF₃CH₂ | O | O | CO₂H | — | — | 0 | N | cyclobenzaprine |
| B1-145 | allyl | O | O | CO₂H | — | — | 0 | N | cyclobenzaprine |
| B1-146 | 4-(NO₂)Ph | O | O | CO₂H | — | — | 0 | N | cyclobenzaprine |
| B1-147 | PhS | O | O | CO₂H | — | — | 0 | N | cyclobenzaprine |
| B1-148 | N(Me)Ph | O | O | CO₂H | — | — | 0 | N | cyclobenzaprine |
| B1-149 | N(H)OMe | O | O | CO₂H | — | — | 0 | N | cyclobenzaprine |
| B1-150 | N(H)CO₂Et | O | O | CO₂H | — | — | 0 | N | cyclobenzaprine |
| B1-151 | Me | O | O | CO₂H | — | — | 0 | N | cyclopentolate |
| B1-152 | cyclohexyl | O | O | CO₂H | — | — | 0 | N | cyclopentolate |
| B1-153 | t-Bu | O | O | CO₂H | — | — | 0 | N | cyclopentolate |
| B1-154 | CF₃CH₂ | O | O | CO₂H | — | — | 0 | N | cyclopentolate |
| B1-155 | allyl | O | O | CO₂H | — | — | 0 | N | cyclopentolate |
| B1-156 | 4-(NO₂)Ph | O | O | CO₂H | — | — | 0 | N | cyclopentolate |
| B1-157 | PhS | O | O | CO₂H | — | — | 0 | N | cyclopentolate |
| B1-158 | N(Me)Ph | O | O | CO₂H | — | — | 0 | N | cyclopentolate |
| B1-159 | N(H)OMe | O | O | CO₂H | — | — | 0 | N | cyclopentolate |
| B1-160 | N(H)CO₂Et | O | O | CO₂H | — | — | 0 | N | cyclopentolate |
| B1-161 | Me | O | O | CO₂H | — | — | 0 | N | dicyclomine |
| B1-162 | cyclohexyl | O | O | CO₂H | — | — | 0 | N | dicyclomine |
| B1-163 | t-Bu | O | O | CO₂H | — | — | 0 | N | dicyclomine |
| B1-164 | CF₃CH₂ | O | O | CO₂H | — | — | 0 | N | dicyclomine |
| B1-165 | allyl | O | O | CO₂H | — | — | 0 | N | dicyclomine |
| B1-166 | 4-(NO₂)Ph | O | O | CO₂H | — | — | 0 | N | dicyclomine |
| B1-167 | PhS | O | O | CO₂H | — | — | 0 | N | dicyclomine |
| B1-168 | N(Me)Ph | O | O | CO₂H | — | — | 0 | N | dicyclomine |
| B1-169 | N(H)OMe | O | O | CO₂H | — | — | 0 | N | dicyclomine |
| B1-170 | N(H)CO₂Et | O | O | CO₂H | — | — | 0 | N | dicyclomine |
| B1-171 | Me | O | O | CO₂H | — | — | 0 | N | diethylproprion |
| B1-172 | cyclohexyl | O | O | CO₂H | — | — | 0 | N | diethylproprion |
| B1-173 | t-Bu | O | O | CO₂H | — | — | 0 | N | diethylproprion |
| B1-174 | CF₃CH₂ | O | O | CO₂H | — | — | 0 | N | diethylproprion |
| B1-175 | allyl | O | O | CO₂H | — | — | 0 | N | diethylproprion |
| B1-176 | 4-(NO₂)Ph | O | O | CO₂H | — | — | 0 | N | diethylproprion |
| B1-177 | PhS | O | O | CO₂H | — | — | 0 | N | diethylproprion |
| B1-178 | N(Me)Ph | O | O | CO₂H | — | — | 0 | N | diethylproprion |
| B1-179 | N(H)OMe | O | O | CO₂H | — | — | 0 | N | diethylproprion |
| B1-180 | N(H)CO₂Et | O | O | CO₂H | — | — | 0 | N | diethylproprion |
| B1-181 | Me | O | O | CO₂H | — | — | 0 | N | diltiazem |
| B1-182 | cyclohexyl | O | O | CO₂H | — | — | 0 | N | diltiazem |
| B1-183 | t-Bu | O | O | CO₂H | — | — | 0 | N | diltiazem |
| B1-184 | CF₃CH₂ | O | O | CO₂H | — | — | 0 | N | diltiazem |
| B1-185 | allyl | O | O | CO₂H | — | — | 0 | N | diltiazem |
| B1-186 | 4-(NO₂)Ph | O | O | CO₂H | — | — | 0 | N | diltiazem |
| B1-187 | PhS | O | O | CO₂H | — | — | 0 | N | diltiazem |
| B1-188 | N(Me)Ph | O | O | CO₂H | — | — | 0 | N | diltiazem |
| B1-189 | N(H)OMe | O | O | CO₂H | — | — | 0 | N | diltiazem |
| B1-190 | N(H)CO2Et | O | O | CO₂H | — | — | 0 | N | diltiazem |
| B1-191 | Me | O | O | CO₂H | — | — | 0 | N | diphenhydramine |
| B1-192 | cyclohexyl | O | O | CO₂H | — | — | 0 | N | diphenhydramine |
| B1-193 | t-Bu | O | O | CO₂H | — | — | 0 | N | diphenhydramine |
| B1-194 | CF₃CH₂ | O | O | CO₂H | — | — | 0 | N | diphenhydramine |
| B1-195 | allyl | O | O | CO₂H | — | — | 0 | N | diphenhydramine |
| B1-196 | 4-(NO₂)Ph | O | O | CO₂H | — | — | 0 | N | diphenhydramine |
| B1-197 | PhS | O | O | CO₂H | — | — | 0 | N | diphenhydramine |
| B1-198 | N(Me)Ph | O | O | CO₂H | — | — | 0 | N | diphenhydramine |
| B1-199 | N(H)OMe | O | O | CO₂H | — | — | 0 | N | diphenhydramine |
| B1-200 | N(H)CO₂Et | O | O | CO₂H | — | — | 0 | N | diphenhydramine |
| B1-201 | Me | O | O | CO₂H | — | — | 0 | N | diphenidol |
| B1-202 | cyclohexyl | O | O | CO₂H | — | — | 0 | N | diphenidol |
| B1-203 | t-Bu | O | O | CO₂H | — | — | 0 | N | diphenidol |
| B1-204 | CF₃CH₂ | O | O | CO₂H | — | — | 0 | N | diphenidol |
| B1-205 | allyl | O | O | CO₂H | — | — | 0 | N | diphenidol |
| B1-206 | 4-(NO₂)Ph | O | O | CO₂H | — | — | 0 | N | diphenidol |
| B1-207 | PhS | O | O | CO₂H | — | — | 0 | N | diphenidol |
| B1-208 | N(Me)Ph | O | O | CO₂H | — | — | 0 | N | diphenidol |
| B1-209 | N(H)OMe | O | O | CO₂H | — | — | 0 | N | diphenidol |
| B1-210 | N(H)CO₂Et | O | O | CO₂H | — | — | 0 | N | diphenidol |
| B1-211 | Me | O | O | CO₂H | — | — | 0 | N | diphenoxylate |
| B1-212 | cyclohexyl | O | O | CO₂H | — | — | 0 | N | diphenoxylate |
| B1-213 | t-Bu | O | O | CO₂H | — | — | 0 | N | diphenoxylate |
| B1-214 | CF₃CH₂ | O | O | CO₂H | — | — | 0 | N | diphenoxylate |
| B1-215 | allyl | O | O | CO₂H | — | — | 0 | N | diphenoxylate |
| B1-216 | 4-(NO₂)Ph | O | O | CO₂H | — | — | 0 | N | diphenoxylate |
| B1-217 | PhS | O | O | CO₂H | — | — | 0 | N | diphenoxylate |
| B1-218 | N(Me)Ph | O | O | CO₂H | — | — | 0 | N | diphenoxylate |
| B1-219 | N(H)OMe | O | O | CO₂H | — | — | 0 | N | diphenoxylate |
| B1-220 | N(H)CO₂Et | O | O | CO₂H | — | — | 0 | N | diphenoxylate |
| B1-221 | Me | O | O | CO₂H | — | — | 0 | N | doxapram |
| B1-222 | cyclohexyl | O | O | CO₂H | — | — | 0 | N | doxapram |
| B1-223 | t-Bu | O | O | CO₂H | — | — | 0 | N | doxapram |
| B1-224 | CF₃CH₂ | O | O | CO₂H | — | — | 0 | N | doxapram |
| B1-225 | allyl | O | O | CO₂H | — | — | 0 | N | doxapram |
| B1-226 | 4-(NO₂)Ph | O | O | CO₂H | — | — | 0 | N | doxapram |
| B1-227 | PhS | O | O | CO₂H | — | — | 0 | N | doxapram |
| B1-228 | N(Me)Ph | O | O | CO₂H | — | — | 0 | N | doxapram |
| B1-229 | N(H)OMe | O | O | CO₂H | — | — | 0 | N | doxapram |
| B1-230 | N(H)CO₂Et | O | O | CO₂H | — | — | 0 | N | doxapram |
| B1-231 | Me | O | O | CO₂H | — | — | 0 | N | doxepin |
| B1-232 | cyclohexyl | O | O | CO₂H | — | — | 0 | N | doxepin |
| B1-233 | t-Bu | O | O | CO₂H | — | — | 0 | N | doxepin |
| B1-234 | CF₃CH₂ | O | O | CO₂H | — | — | 0 | N | doxepin |
| B1-235 | allyl | O | O | CO₂H | — | — | 0 | N | doxepin |
| B1-236 | 4-(NO₂)Ph | O | O | CO₂H | — | — | 0 | N | doxepin |
| B1-237 | PhS | O | O | CO₂H | — | — | 0 | N | doxepin |
| B1-238 | N(Me)Ph | O | O | CO₂H | — | — | 0 | N | doxepin |
| B1-239 | N(H)OMe | O | O | CO₂H | — | — | 0 | N | doxepin |
| B1-240 | N(H)CO₂Et | O | O | CO₂H | — | — | 0 | N | doxepin |
| B1-241 | Me | O | O | CO₂H | — | — | 0 | N | fentanyl |
| B1-242 | cyclohexyl | O | O | CO₂H | — | — | 0 | N | fentanyl |
| B1-243 | t-Bu | O | O | CO₂H | — | — | 0 | N | fentanyl |
| B1-244 | CF₃CH₂ | O | O | CO₂H | — | — | 0 | N | fentanyl |
| B1-245 | allyl | O | O | CO₂H | — | — | 0 | N | fentanyl |
| B1-246 | 4-(NO₂)Ph | O | O | CO₂H | — | — | 0 | N | fentanyl |
| B1-247 | PhS | O | O | CO₂H | — | — | 0 | N | fentanyl |
| B1-248 | N(Me)Ph | O | O | CO₂H | — | — | 0 | N | fentanyl |
| B1-249 | N(H)OMe | O | O | CO₂H | — | — | 0 | N | fentanyl |
| B1-250 | N(H)CO₂Et | O | O | CO₂H | — | — | 0 | N | fentanyl |
| B1-251 | Me | O | O | CO₂H | — | — | 0 | N | flavoxate |
| B1-252 | cyclohexyl | O | O | CO₂H | — | — | 0 | N | flavoxate |
| B1-253 | t-Bu | O | O | CO₂H | — | — | 0 | N | flavoxate |
| B1-254 | CF₃CH₂ | O | O | CO₂H | — | — | 0 | N | flavoxate |
| B1-255 | allyl | O | O | CO₂H | — | — | 0 | N | flavoxate |
| B1-256 | 4-(NO₂)Ph | O | O | CO₂H | — | — | 0 | N | flavoxate |
| B1-257 | PhS | O | O | CO₂H | — | — | 0 | N | flavoxate |
| B1-258 | N(Me)Ph | O | O | CO₂H | — | — | 0 | N | flavoxate |
| B1-259 | N(H)OMe | O | O | CO₂H | — | — | 0 | N | flavoxate |
| B1-260 | N(H)CO2Et | O | O | CO₂H | — | — | 0 | N | flavoxate |
| B1-261 | Me | O | O | CO₂H | — | — | 0 | N | flurazepam |
| B1-262 | cyclohexyl | O | O | CO₂H | — | — | 0 | N | flurazepam |
| B1-263 | t-Bu | O | O | CO₂H | — | — | 0 | N | flurazepam |
| B1-264 | CF₃CH₂ | O | O | CO₂H | — | — | 0 | N | flurazepam |
| B1-265 | allyl | O | O | CO₂H | — | — | 0 | N | flurazepam |
| B1-266 | 4-(NO₂)Ph | O | O | CO₂H | — | — | 0 | N | flurazepam |
| B1-267 | PhS | O | O | CO₂H | — | — | 0 | N | flurazepam |
| B1-268 | N(Me)Ph | O | O | CO₂H | — | — | 0 | N | flurazepam |
| B1-269 | N(H)OMe | O | O | CO₂H | — | — | 0 | N | flurazepam |
| B1-270 | N(H)CO₂Et | O | O | CO₂H | — | — | 0 | N | flurazepam |
| B1-271 | Me | O | O | CO₂H | — | — | 0 | N | levomethadyl |
| B1-272 | cyclohexyl | O | O | CO₂H | — | — | 0 | N | levomethadyl |
| B1-273 | t-Bu | O | O | CO₂H | — | — | 0 | N | levomethadyl |
| B1-274 | CF₃CH₂ | O | O | CO₂H | — | — | 0 | N | levomethadyl |
| B1-275 | allyl | O | O | CO₂H | — | — | 0 | N | levomethadyl |
| B1-276 | 4-(NO₂)Ph | O | O | CO₂H | — | — | 0 | N | levomethadyl |
| B1-277 | PhS | O | O | CO₂H | — | — | 0 | N | levomethadyl |
| B1-278 | N(Me)Ph | O | O | CO₂H | — | — | 0 | N | levomethadyl |
| B1-279 | N(H)OMe | O | O | CO₂H | — | — | 0 | N | levomethadyl |
| B1-280 | N(H)CO₂Et | O | O | CO₂H | — | — | 0 | N | levomethadyl |
| B1-281 | Me | O | O | CO₂H | — | — | 0 | N | loratadine |
| B1-282 | cyclohexyl | O | O | CO₂H | — | — | 0 | N | loratadine |
| B1-283 | t-Bu | O | O | CO₂H | — | — | 0 | N | loratadine |
| B1-284 | CF₃CH₂ | O | O | CO₂H | — | — | 0 | N | loratadine |
| B1-285 | allyl | O | O | CO₂H | — | — | 0 | N | loratadine |
| B1-286 | 4-(NO₂)Ph | O | O | CO₂H | — | — | 0 | N | loratadine |
| B1-287 | PhS | O | O | CO₂H | — | — | 0 | N | loratadine |
| B1-288 | N(Me)Ph | O | O | CO₂H | — | — | 0 | N | loratadine |
| B1-289 | N(H~OMe | O | O | CO₂H | — | — | 0 | N | loratadine |
| B1-290 | N(H)CO2Et | O | O | COdI | — | — | 0 | N | loratadine |
| B1-291 | Me | O | O | CO₂H | — | — | 0 | N | mechlorethamine |
| B1-292 | cyclohexyl | O | O | CO₂H | — | — | 0 | N | mechlorethamine |
| B1-293 | t-Bu | O | O | CO₂H | — | — | 0 | N | mechlorethamine |
| B1-294 | CF₃CH₂ | O | O | CO₂H | — | — | 0 | N | mechlorethamine |
| B1-295 | allyl | O | O | CO₂H | — | — | 0 | N | mechlorethamine |
| B1-296 | 4-(NO₂)Ph | O | O | CO₂H | — | — | 0 | N | mechlorethamine |
| B1-297 | PhS | O | O | CO₂H | — | — | 0 | N | mechlorethamine |
| B1-298 | N(Me)Ph | O | O | CO₂H | — | — | 0 | N | mechlorethamine |
| B1-299 | N(H)OMe | O | O | CO₂H | — | — | 0 | N | mechlorethamine |
| B1-300 | N(H)CO2Et | O | O | CO₂H | — | — | 0 | N | mechlorethamine |
| B1-301 | Me | O | O | CO₂H | — | — | 0 | N | meperidine |
| B1-302 | cyclohexyl | O | O | CO₂H | — | — | 0 | N | meperidine |
| B1-303 | t-Bu | O | O | CO₂H | — | — | 0 | N | meperidine |
| B1-304 | CF₃CH₂ | O | O | COdI | — | — | 0 | N | meperidine |
| B1-305 | allyl | O | O | CO₂H | — | — | 0 | N | meperidine |
| B1-306 | 4-(NO₂)Ph | O | O | CO₂H | — | — | 0 | N | meperidine |
| B1-307 | PhS | O | O | CO₂H | — | — | 0 | N | meperidine |
| B1-308 | N(Me)Ph | O | O | CO₂H | — | — | 0 | N | meperidine |
| B1-309 | N(H)OMe | O | O | CO₂H | — | — | 0 | N | meperidine |
| B1-310 | N(H)CO₂Et | O | O | CO₂H | — | — | 0 | N | meperidine |
| B1-311 | Me | O | O | CO₂H | — | — | 0 | N | mepivacaine |
| B1-312 | cyclohexyl | O | O | CO₂H | — | — | 0 | N | mepivacaine |
| B1-313 | t-Bu | O | O | CO₂H | — | — | 0 | N | mepivacaine |
| B1-314 | CF₃CH₂ | O | O | CO₂H | — | — | 0 | N | mepivacaine |
| B1-315 | allyl | O | O | CO₂H | — | — | 0 | N | mepivacaine |
| B1-316 | 4-(NO₂)Ph | O | O | CO₂H | — | — | 0 | N | mepivacaine |
| B1-317 | PhS | O | O | CO₂H | — | — | 0 | N | mepivacaine |
| B1-318 | N(Me)Ph | O | O | CO₂H | — | — | 0 | N | mepivacaine |
| B1-319 | N(H)OMe | O | O | CO₂H | — | — | 0 | N | mepivacaine |
| B1-320 | N(H)CO₂Et | O | O | CO₂H | — | — | 0 | N | mepivacaine |
| B1-321 | Me | O | O | CO₂H | — | — | 0 | N | methadone |
| B1-322 | cyclohexyl | O | O | CO₂H | — | — | 0 | N | methadone |
| B1-323 | t-Bu | O | O | CO₂H | — | — | 0 | N | methadone |
| B1-324 | CF₃CH₂ | O | O | CO₂H | — | — | 0 | N | methadone |
| B1-325 | allyl | O | O | CO₂H | — | — | 0 | N | methadone |
| B1-326 | 4-(NO₂)Ph | O | O | CO₂H | — | — | 0 | N | methadone |
| B1-327 | PhS | O | O | CO₂H | — | — | 0 | N | methadone |
| B1-328 | N(Me)Ph | O | O | CO₂H | — | — | 0 | N | methadone |
| B1-329 | N(H)OMe | O | O | CO₂H | — | — | 0 | N | methadone |
| B1-330 | N(H)CO₂Et | O | O | CO₂H | — | — | 0 | N | methadone |
| B1-331 | Me | O | O | CO₂H | — | — | 0 | N | minoxidil |
| B1-332 | cyclohexyl | O | O | CO₂H | — | — | 0 | N | minoxidil |
| B1-333 | t-Bu | O | O | CO₂H | — | — | 0 | N | minoxidil |
| B1-334 | CF₃CH₂ | O | O | CO₂H | — | — | 0 | N | minoxidil |
| B1-335 | allyl | O | O | CO₂H | — | — | 0 | N | minoxidil |
| B1-336 | 4-(NO₂)Ph | O | O | CO₂H | — | — | 0 | N | minoxidil |
| B1-337 | PhS | O | O | CO₂H | — | — | 0 | N | minoxidil |
| B1-338 | N(Me)Ph | O | O | CO₂H | — | — | 0 | N | minoxidil |
| B1-339 | N(H)OMe | O | O | CO₂H | — | — | 0 | N | minoxidil |
| B1-340 | N(H)CO₂Et | O | O | CO₂H | — | — | 0 | N | minoxidil |
| B1-341 | Me | O | O | CO₂H | — | — | 0 | N | naftifine |
| B1-342 | cyclohexyl | O | O | CO₂H | — | — | 0 | N | naftifine |
| B1-343 | t-Bu | O | O | CO₂H | — | — | 0 | N | naftifine |
| B1-344 | CF₃CH₂ | O | O | CO₂H | — | — | 0 | N | naftifine |
| B1-345 | allyl | O | O | CO₂H | — | — | 0 | N | naftifine |
| B1-346 | 4-(NO₂)Ph | O | O | CO₂H | — | — | 0 | N | naftifine |
| B1-347 | PhS | O | O | CO₂H | — | — | 0 | N | naftifine |
| B1-348 | N(Me)Ph | O | O | CO₂H | — | — | 0 | N | naftifine |
| B1-349 | N(H)OMe | O | O | CO₂H | — | — | 0 | N | naftifine |
| B1-350 | N(H)CO₂Et | O | O | CO₂H | — | — | 0 | N | naftifine |
| B1-351 | Me | O | O | CO₂H | — | — | 0 | N | orphenadrine |
| B1-352 | cyclohexyl | O | O | CO₂H | — | — | 0 | N | orphenadrine |
| B1-353 | t-Bu | O | O | CO₂H | — | — | 0 | N | orphenadrine |
| B1-354 | CF₃CH₂ | O | O | CO₂H | — | — | 0 | N | orphenadrine |
| B1-355 | ally1 | O | O | CO₂H | — | — | 0 | N | orphenadrine |
| B1-356 | 4-(NO₂)Ph | O | O | CO₂H | — | — | 0 | N | orphenadrine |
| B1-357 | PhS | O | O | CO₂H | — | — | 0 | N | orphenadrine |
| B1-358 | N(Me)Ph | O | O | CO₂H | — | — | 0 | N | orphenadrine |
| B1-359 | N(H)OMe | O | O | CO₂H | — | — | 0 | N | orphenadrine |
| B1-360 | N(H)CO₂Et | O | O | CO₂H | — | — | 0 | N | orphenadrine |
| B1-361 | Me | O | O | CO₂H | — | — | 0 | N | oxybutynin |
| B1-362 | cyclohexyl | O | O | CO₂H | — | — | 0 | N | oxybutynin |
| B1-363 | t-Bu | O | O | CO₂H | — | — | 0 | N | oxybutynin |
| B1-364 | CF₃CH₂ | O | O | CO₂H | — | — | 0 | N | oxybutynin |
| B1-365 | allyl | O | O | CO₂H | — | — | 0 | N | oxybutynin |
| B1-366 | 4-(NO₂)Ph | O | O | CO₂H | — | — | 0 | N | oxybutynin |
| B1-367 | PhS | O | O | CO₂H | — | — | 0 | N | oxybutynin |
| B1-368 | N(Me)Ph | O | O | CO₂H | — | — | 0 | N | oxybutynin |
| B1-369 | N(H)OMe | O | O | CO₂H | — | — | 0 | N | oxybutynin |
| B1-370 | N(H)CO₂Et | O | O | CO₂H | — | — | 0 | N | oxybutynin |
| B1-371 | Me | O | O | CO₂H | — | — | 0 | N | oxymetazoline |
| B1-372 | cyclohexyl | O | O | CO₂H | — | — | 0 | N | oxymetazohne |
| B1-373 | t-Bu | O | O | CQ2H | — | — | 0 | N | oxymetazoline |
| B1-374 | CF₃CH₂ | O | O | CO₂H | — | — | 0 | N | oxymetazoline |
| B1-375 | allyl | O | O | CO₂H | — | — | 0 | N | oxymetazoline |
| B1-376 | 4-(NO₂)Ph | O | O | CO₂H | — | — | 0 | N | oxymetazoline |
| B1-377 | PhS | O | O | CO₂H | — | — | 0 | N | oxymetazoline |
| B1-378 | N(Me)Ph | O | O | CO₂H | — | — | 0 | N | oxymetazoline |
| B1-379 | N(H)OMe | O | O | CO₂H | — | — | 0 | N | oxymetazoline |
| B1-380 | N(H)CO2Et | O | O | CO₂H | — | — | 0 | N | oxymetazoline |
| B1-381 | Me | O | O | CO₂H | — | — | 0 | N | phenoxybenzamine |
| B1-382 | cyclohexyl | O | O | CO₂H | — | — | 0 | N | phenoxybenzamine |
| B1-383 | t-Bu | O | O | CO₂H | — | — | 0 | N | phenoxybenzamine |
| B1-384 | CF₃CH₂ | O | O | CO₂H | — | — | 0 | N | phenoxybenzamine |
| B1-385 | allyl | O | O | CO₂H | — | — | 0 | N | phenoxybenzamine |
| B1-386 | 4-(NO₂)Ph | O | O | CO₂H | — | — | 0 | N | phenoxybenzamine |
| B1-387 | PhS | O | O | CO₂H | — | — | 0 | N | phenoxybenzamine |
| B1-388 | N(Me)Ph | O | O | CO₂H | — | — | 0 | N | phenoxybenzamine |
| B1-389 | N(H)OMe | O | O | CO₂H | — | — | 0 | N | phenoxybenzamine |
| B1-390 | N(H)CO₂Et | O | O | CO₂H | — | — | 0 | N | phenoxybenzamine |
| B1-391 | Me | O | O | CO₂H | — | — | 0 | N | pilocarpine |
| B1-392 | cyclohexyl | O | O | CO₂H | — | — | 0 | N | pilocarpine |
| B1-393 | t-Bu | O | O | CO₂H | — | — | 0 | N | pilocarpine |
| B1-394 | CF₃CH₂ | O | O | CO₂H | — | — | 0 | N | pilocarpine |
| B1-395 | allyl | O | O | CO₂H | — | — | 0 | N | pilocarpine |
| B1-396 | 4-(NO₂)Ph | O | O | CO₂H | — | — | 0 | N | pilocarpine |
| B1-397 | PhS | O | O | CO₂H | — | — | 0 | N | pilocarpine |
| B1-398 | N(Me)Ph | O | O | CO₂H | — | — | 0 | N | pilocarpine |
| B1-399 | N(H)OMe | O | O | CO₂H | — | — | 0 | N | pilocarpine |
| B1-400 | N(H)CO₂Et | O | O | CO₂H | — | — | 0 | N | pilocarpine |
| B1-401 | Me | O | O | CO₂H | — | — | 0 | N | pyrazinamide |
| B1-402 | cyclohexyl | O | O | CO₂H | — | — | 0 | N | pyrazinamide |
| B1-403 | t-Bu | O | O | CO₂H | — | — | 0 | N | pyrazinamide |
| B1-404 | CF₃CH₂ | O | O | CO₂H | — | — | 0 | N | pyrazinamide |
| B1-405 | allyl | O | O | CO₂H | — | — | 0 | N | pyrazinamide |
| B1-406 | 4-(NO₂)Ph | O | O | CO₂H | — | — | 0 | N | pyrazinamide |
| B1-407 | PhS | O | O | CO₂H | — | — | 0 | N | pyrazinamide |
| B1-408 | N(Me)Ph | O | O | CO₂H | — | — | 0 | N | pyrazinamide |
| B1-409 | N(H)OMe | O | O | CO₂H | — | — | 0 | N | pyrazinamide |
| B1-410 | N(H)CO₂Et | O | O | CO₂H | — | — | 0 | N | pyrazinamide |
| B1-411 | Me | O | O | CO₂H | — | — | 0 | N | pyroxidine |
| B1-412 | cyclohexyl | O | O | CO₂H | — | — | 0 | N | pyroxidine |
| BL-413 | t-Bu | O | O | CO₂H | — | — | 0 | N | pyroxidine |
| B1-414 | CF₃CH₂ | O | O | CO₂H | — | — | 0 | N | pyroxidine |
| B1-415 | allyl | O | O | CO₂H | — | — | 0 | N | pyroxidine |
| B1-416 | 4-(NO₂)Ph | O | O | CO₂H | — | — | 0 | N | pyroxidine |
| B1-417 | PhS | O | O | CO₂H | — | — | 0 | N | pyroxidine |
| B1-418 | N(Me)Ph | O | O | CO₂H | — | — | 0 | N | pyroxidine |
| B1-419 | N(H)OMe | O | O | CO₂H | — | — | 0 | N | pyroxidine |
| B1-420 | N(H)CO₂Et | O | O | CO₂H | — | — | 0 | N | pyroxidine |
| BL-421 | Me | O | O | CO₂H | — | — | 0 | N | risperidone |
| B1-422 | cyclohexyl | O | O | CO₂H | — | — | 0 | N | risperidone |
| B1-423 | t-Bu | O | O | CO₂H | — | — | 0 | N | risperidone |
| B1-424 | CF₃CH₂ | O | O | CO₂H | — | — | 0 | N | risperidone |
| B1-425 | allyl | O | O | CO₂H | — | — | 0 | N | risperidone |
| B1-426 | 4-(NO₂)Ph | O | O | CO₂H | — | — | 0 | N | risperidone |
| B1-427 | PhS | O | O | CO₂H | — | — | 0 | N | risperidone |
| B1-428 | N(Me)Ph | O | O | CO₂H | — | — | 0 | N | risperidone |
| B1-429 | N(H)OMe | O | O | CO₂H | — | — | 0 | N | risperidone |
| B1-430 | N(H)CO₂Et | O | O | CO₂H | — | — | 0 | N | risperidone |
| BL-431 | Me | O | O | CO₂H | — | — | 0 | N | sufentanil |
| B1-432 | cyclohexyl | O | O | CO₂H | — | — | 0 | N | sufentanil |
| B1-433 | t-Bu | O | O | CO₂H | — | — | 0 | N | sufentanil |
| B1-434 | CF₃CH₂ | O | O | CO₂H | — | — | 0 | N | sufentanil |
| B1-435 | allyl | O | O | CO₂H | — | — | 0 | N | sufentanil |
| B1-436 | 4-(NO₂)Ph | O | O | CO₂H | — | — | 0 | N | sufentanil |
| B1-437 | PhS | O | O | CO₂H | — | — | 0 | N | sufentanil |
| B1-438 | N(Me)Ph | O | O | CO₂H | — | — | 0 | N | sufentanil |
| B1-439 | N(H)OMe | O | O | CO₂H | — | — | 0 | N | sufentanil |
| B1-440 | N(H)CO₂Et | O | O | CO₂H | — | — | 0 | N | sufentanil |
| B1-441 | Me | O | O | CO₂H | — | — | 0 | N | tamoxifen |
| B1-442 | cyclohexyl | O | O | CO₂H | — | — | 0 | N | tamoxifen |
| B1-443 | t-Bu | O | O | CO₂H | — | — | 0 | N | tamoxifen |
| B1-444 | CF₃CH₂ | O | O | CO₂H | — | — | 0 | N | tamoxifen |
| B1-445 | allyl | O | O | CO₂H | — | — | 0 | N | tamoxifen |
| B1-446 | 4-(NO₂)Ph | O | O | CO₂H | — | — | 0 | N | tamoxifen |
| B1-447 | PhS | O | O | CO₂H | — | — | 0 | N | tamoxifen |
| B1-448 | N(Me)Ph | O | O | CO₂H | — | — | 0 | N | tamoxifen |
| B1-449 | N(H)OMe | O | O | CO₂H | — | — | 0 | N | tamoxifen |
| B1-450 | N(H)CO₂Et | O | O | CO₂H | — | — | 0 | N | tamoxifen |
| B1-451 | Me | O | O | CO₂H | — | — | 0 | N | terbinafine |
| B1-452 | cyclohexyl | O | O | CO₂H | — | — | 0 | N | terbinafine |
| B1-453 | t-Bu | O | O | CO₂H | — | — | 0 | N | terbinafine |
| B1-454 | CF₃CH₂ | O | O | CO₂H | — | — | 0 | N | terbinafine |
| B1-455 | allyl | O | O | CO₂H | — | — | 0 | N | terbinafine |
| B1-456 | 4-(NO₂)Ph | O | O | CO₂H | — | — | 0 | N | terbinafine |
| B1-457 | PhS | O | O | CO₂H | — | — | 0 | N | terbinafine |
| B1-458 | N(Me)Ph | O | O | CO₂H | — | — | 0 | N | terbinafine |
| B1-459 | N(H)OMe | O | O | CO₂H | — | — | 0 | N | terbinafine |
| B1-460 | N(H)CO₂Et | O | O | CO₂H | — | — | 0 | N | terbinafine |
| B1-461 | Me | O | O | CO₂H | — | — | 0 | N | trihexyphenidyl |
| B1-462 | cyclohexyl | O | O | CO₂H | — | — | 0 | N | trihexyphenidyl |
| B1-463 | t-Bu | O | O | CO₂H | — | — | 0 | N | trihexyphenidyl |
| B1-464 | CF₃CH₂ | O | O | CO₂H | — | — | 0 | N | trihexyphenidyl |
| B1-465 | allyl | O | O | CO₂H | — | — | 0 | N | trihexyphenidyl |
| B1-466 | 4-(NO₂)Ph | O | O | CO₂H | — | — | 0 | N | trihexyphenidyl |
| B1-467 | PhS | O | O | CO₂H | — | — | 0 | N | trihexyphenidyl |
| B1-468 | N(Me)Ph | O | O | CO₂H | — | — | 0 | N | trihexyphenidyl |
| B1-469 | N(H)OMe | O | O | CO₂H | — | — | 0 | N | trihexyphenidyl |
| B1-470 | N(H)CO₂Et | O | O | CO₂H | — | — | 0 | N | trihexyphenidyl |
| B1-471 | Me | O | O | CO₂H | — | — | 0 | N | troleandomycin |
| B1-472 | cyclohexyl | O | O | CO₂H | — | — | 0 | N | troleandomycin |
| B1-473 | t-Bu | O | O | CO₂H | — | — | 0 | N | troleandomycin |
| B1-474 | CF₃CH₂ | O | O | CO₂H | — | — | 0 | N | troleandomycin |
| B1-475 | allyl | O | O | CO₂H | — | — | 0 | N | troleandomycin |
| B1-476 | 4-(NO₂)Ph | O | O | CO₂H | — | — | 0 | N | troleandomycin |
| B1-477 | PhS | O | O | CO₂H | — | — | 0 | N | troleandomycin |
| B1-478 | N(Me)Ph | O | O | CO₂H | — | — | 0 | N | troleandomycin |
| B1-479 | N(H)OMe | O | O | CO₂H | — | — | 0 | N | troleandomycin |
| B1-480 | N(H)CO₂Et | O | O | CO₂H | — | — | 0 | N | troleandomycin |
| B1-481 | Me | O | O | CO₂H | — | — | 0 | N | verapamil |
| B1-482 | cyclohexyl | O | O | CO₂H | — | — | 0 | N | verapamil |
| B1-483 | t-Bu | O | O | CO₂H | — | — | 0 | N | verapamil |
| B1-484 | CF₃CH₂ | O | O | CO₂H | — | — | 0 | N | verapamil |
| B1-485 | allyl | O | O | CO₂H | — | — | 0 | N | verapamil |
| B1-486 | 4-(NO₂)Ph | O | O | CO₂H | — | — | 0 | N | verapamil |
| B1-487 | PhS | O | O | CO₂H | — | — | 0 | N | verapamil |
| B1-488 | N(Me)Ph | O | O | CO₂H | — | — | 0 | N | verapamil |
| B1-489 | N(H)OMe | O | O | CO₂H | — | — | 0 | N | verapamil |
| B1-490 | N(H)CO₂Et | O | O | CO₂H | — | — | 0 | N | verapamil |
| B1-491 | Me | O | O | CO₂H | — | — | 0 | N | caffeine |
| B1-492 | cyclohexyl | O | O | CO₂H | — | — | 0 | N | caffeine |
| B1-493 | t-Bu | O | O | CO₂H | — | — | 0 | N | caffeine |
| B1-494 | CF₃CH₂ | O | O | CO₂H | — | — | 0 | N | caffeine |
| B1-495 | allyl | O | O | CO₂H | — | — | 0 | N | caffeine |
| B1-496 | 4-(NO₂)Ph | O | O | CO₂H | — | — | 0 | N | caffeine |
| B1-497 | PhS | O | O | CO₂H | — | — | 0 | N | caffeine |
| B1-498 | N(Me)Ph | O | O | CO₂H | — | — | 0 | N | caffeine |
| B1-499 | N(H)OMe | O | O | CO₂H | — | — | 0 | N | caffeine |
| B1-500 | N(H)CO₂Et | O | O | CO₂H | — | — | 0 | N | caffeine |
| B1-501 | Me | O | O | CO₂H | — | — | 0 | N | cyproheptadine |
| B1-502 | cyclohexyl | O | O | CO₂H | — | — | 0 | N | cyproheptadine |
| B1-503 | t-Bu | O | O | CO₂H | — | — | 0 | N | cyproheptadine |
| B1-504 | CF₃CH₂ | O | O | CO₂H | — | — | 0 | N | cyproheptadine |
| B1-505 | allyl | O | O | CO₂H | — | — | 0 | N | cyproheptadine |
| B1-506 | 4-(NO₂)Ph | O | O | CO₂H | — | — | 0 | N | cyproheptadine |
| B1-507 | PhS | O | O | CO₂H | — | — | 0 | N | cyproheptadine |
| B1-508 | N(Me)Ph | O | O | CO₂H | — | — | 0 | N | cyproheptadine |
| B1-509 | N(H)OMe | O | O | CO₂H | — | — | 0 | N | cyproheptadine |
| B1-510 | N(H)CO₂Et | O | O | CO₂H | — | — | 0 | N | cyproheptadine |
| B1-511 | Me | O | O | CO₂H | — | — | 0 | N | pramoxine |
| B1-512 | cyclohexyl | O | O | CO₂H | — | — | 0 | N | pramoxine |
| B1-513 | t-Bu | O | O | CO₂H | — | — | 0 | N | pramoxine |
| B1-514 | CF₃CH₂ | O | O | CO₂H | — | — | 0 | N | pramoxine |
| B1-515 | allyl | O | O | CO₂H | — | — | 0 | N | pramoxine |
| B1-516 | 4-(NO₂)Ph | O | O | CO₂H | — | — | 0 | N | pramoxine |
| B1-517 | PhS | O | O | CO₂H | — | — | 0 | N | praniioxine |
| B1-518 | N(Me)Ph | O | O | CO₂H | — | — | 0 | N | pramoxine |
| B1-519 | N(H)OMe | O | O | CO₂H | — | — | 0 | N | pramoxine |
| B1-520 | N(H)CO₂Et | O | O | CO₂H | — | — | 0 | N | pramoxine |
| B1-521 | Me | O | O | CO₂H | — | — | 0 | O | iodoquinol |
| B1-522 | cyclohexyl | O | O | CO₂H | — | — | 0 | O | iodoquinol |
| B1-523 | t-Bu | O | O | CO₂H | — | — | 0 | O | iodoquinol |
| B1-524 | CF₃CH₂ | O | O | CO₂H | — | — | 0 | O | iodoquinol |
| B1-525 | allyl | O | O | CO₂H | — | — | 0 | O | iodoquinol |
| B1-526 | 4-(NO₂)Ph | O | O | CO₂H | — | — | 0 | O | iodoquinol |
| B1-527 | PhS | O | O | CO₂H | — | — | 0 | O | iodoquinol |
| B1-528 | N(Me)Ph | O | O | CO₂H | — | — | 0 | O | iodoquinol |
| B1-529 | N(H)OMe | O | O | CO₂H | — | — | 0 | O | iodoquinol |
| B1-530 | N(H)CO₂Et | O | O | CO₂H | — | — | 0 | O | iodoquinol |
| B1-531 | Me | O | O | CO₂H | — | — | 0 | O | metronidazole |
| B1-532 | cyclohexyl | O | O | CO₂H | — | — | 0 | O | metronidazole |
| B1-533 | t-Bu | O | O | CO₂H | — | — | 0 | O | metronidazole |
| B1-534 | CF₃CH₂ | O | O | CO₂H | — | — | 0 | O | metronidazole |
| B1-535 | allyl | O | O | CO₂H | — | — | 0 | O | metronidazole |
| B1-536 | 4-(NO₂)Ph | O | O | CO₂H | — | — | 0 | O | metronidazole |
| B1-537 | PhS | O | O | CO₂H | — | — | 0 | O | metronidazole |
| B1-538 | N(Me)Ph | O | O | CO₂H | — | — | 0 | O | metronidazole |
| B1-539 | N(H)OMe | O | O | CO₂H | — | — | 0 | O | metronidazole |
| B1-540 | N(H)CO₂Et | O | O | CO₂H | — | — | 0 | O | metronidazole |
| B1-541 | Me | O | O | CO₂H | — | — | 0 | N | papaverine |
| B1-542 | cyclohexyl | O | O | CO₂H | — | — | 0 | N | papaverine |
| B1-543 | t-Bu | O | O | CO₂H | — | — | 0 | N | papaverine |
| B1-544 | CF₃CH₂ | O | O | CO₂H | — | — | 0 | N | papaverine |
| B1-545 | allyl | O | O | CO₂H | — | — | 0 | N | papaverine |
| B1-546 | 4-(NO₂)Ph | O | O | CO₂H | — | — | 0 | N | papaverine |
| B1-547 | PhS | O | O | CO₂H | — | — | 0 | N | papaverine |
| B1-548 | N(Me)Ph | O | O | CO₂H | — | — | 0 | N | papaverine |
| B1-549 | N(H)OMe | O | O | CO₂H | — | — | 0 | N | papaverine |
| B1-550 | N(H)CO₂Et | O | O | CO₂H | — | — | 0 | N | papaverine |
| B1-551 | Me | O | O | CO₂H | — | — | 0 | N | tropicamide |
| B1-552 | cyclohexyl | O | O | CO₂H | — | — | 0 | N | tropicamide |
| B1-553 | t-Bu | O | O | CO₂H | — | — | 0 | N | tropicamide |
| B1-554 | CF₃CH₂ | O | O | CO₂H | — | — | 0 | N | tropicamide |
| B1-555 | allyL | O | O | CO₂H | — | — | 0 | N | tropicamide |
| B1-556 | 4-(NO₂)Ph | O | O | CO₂H | — | — | 0 | N | tropicamide |
| B1-557 | PhS | O | O | CO₂H | — | — | 0 | N | tropicamide |
| B1-558 | N(Me)Ph | O | O | CO₂H | — | — | 0 | N | tropicamide |
| B1-559 | N(H)OMe | O | O | CO₂H | — | — | 0 | N | tropicamide |
| B1-560 | N(H)CO₂Et | O | O | CO₂H | — | — | 0 | N | tropicamide |
| B1-561 | Me | O | O | CO₂H | — | — | 0 | N | halazepam |
| B1-562 | cyclohexyl | O | O | CO₂H | — | — | 0 | N | halazepam |
| B1-563 | t-Bu | O | O | CO₂H | — | — | 0 | N | halazepam |
| B1-564 | CF₃CH₂ | O | O | CO₂H | — | — | 0 | N | halazepam |
| B1-565 | allyl | O | O | CO₂H | — | — | 0 | N | halazepam |
| B1-566 | 4-(NO₂)Ph | O | O | CO₂H | — | — | 0 | N | halazepam |
| B1-567 | PhS | O | O | CO₂H | — | — | 0 | N | halazepam |
| B1-568 | N(Me)Ph | O | O | CO₂H | — | — | 0 | N | halazepam |
| B1-569 | N(H)OMe | O | O | CO₂H | — | — | 0 | N | halazepam |
| B1-570 | N(H)CO₂Et | O | O | CO₂H | — | — | 0 | N | halazepam |
| B1-571 | Me | O | O | CO₂H | — | — | 0 | O | mazindol |
| B1-572 | cyclohexyl | O | O | CO₂H | — | — | 0 | O | mazindol |
| B1-573 | t-Bu | O | O | CO₂H | — | — | 0 | O | mazindol |
| B1-574 | CF₃CH₂ | O | O | CO₂H | — | — | 0 | O | mazindol |
| B1-575 | allyl | O | O | CO₂H | — | — | 0 | O | mazindol |
| B1-576 | 4-(NO₂)Ph | O | O | CO₂H | — | — | 0 | O | mazindol |
| B1-577 | PhS | O | O | CO₂H | — | — | 0 | O | mazindol |
| B1-578 | N(Me)Ph | O | O | CO₂H | — | — | 0 | O | mazindol |
| B1-579 | N(H)OMe | O | O | CO₂H | — | — | 0 | O | mazindol |
| B1-580 | N(H)CO₂Et | O | O | CO₂H | — | — | 0 | O | mazindol |
| B1-581 | Me | O | O | CO₂H | — | — | 0 | O | hydroxyitraconazole |
| B1-582 | cyclohexyl | O | O | CO₂H | — | — | 0 | O | hydroxyitraconazole |
| B1-583 | t-Bu | O | O | CO₂H | — | — | 0 | O | hydroxyitraconazole |
| B1-584 | CF₃CH₂ | O | O | CO₂H | — | — | 0 | O | hydroxyitraconazole |
| B1-585 | allyl | O | O | CO₂H | — | — | 0 | O | hydroxyitraconazole |
| B1-586 | 4-(NO₂)Ph | O | O | CO₂H | — | — | 0 | O | hydroxyitraconazole |
| B1-587 | PhS | O | O | CO₂H | — | — | 0 | O | hydroxyitraconazole |
| B1-588 | N(Me)Ph | O | O | CO₂H | — | — | 0 | O | hydroxyitraconazole |
| B1-589 | N(H)OMe | O | O | CO₂H | — | — | 0 | O | hydroxyitraconazole |
| B1-590 | N(H)CO₂Et | O | O | CO₂H | — | — | 0 | O | hydroxyitraconazole |
| B1-591 | Me | O | O | CO₂H | — | — | 0 | O | posaconazole |
| B1-593 | t-Bu | O | O | CO₂H | — | — | 0 | O | posaconazole |
| B1-594 | CF₃CH₂ | O | O | COdI | — | — | 0 | O | posaconazole |
| B1-595 | allyl | O | O | CO₂H | — | — | 0 | O | posaconazole |
| B1-596 | 4-(NO₂)Ph | O | O | CO₂H | — | — | 0 | O | posaconazole |
| B1-597 | PhS | O | O | CO₂H | — | — | 0 | O | posaconazole |
| B1-598 | N(Me)Ph | O | O | CO₂H | — | — | 0 | O | posaconazole |
| B1-599 | N(H)OMe | O | O | CO₂H | — | — | 0 | O | posaconazole |
| B1-600 | N(H)CO₂Et | O | O | CO₂H | — | — | 0 | O | posaconazole |
| B1-601 | Me | O | O | CO₂H | — | — | 0 | O | voriconazole |
| B1-602 | cyclohexyl | O | O | CO₂H | — | — | 0 | O | voriconazole |
| B1-603 | t-Bu | O | O | CO₂H | — | — | 0 | O | voriconazole |
| B1-604 | CF₃CH₂ | O | O | CO₂H | — | — | 0 | O | voriconazole |
| B1-605 | allyl | O | O | CO₂H | — | — | 0 | O | voriconazole |
| B1-606 | 4-(NO₂)Ph | O | O | CO₂H | — | — | 0 | O | voriconazole |
| B1-607 | PhS | O | O | CO₂H | — | — | 0 | O | voriconazole |
| B1-608 | N(Me)Ph | O | O | CO₂H | — | — | 0 | O | voriconazole |
| B1-609 | N(H)OMe | O | O | CO₂H | — | — | 0 | O | voriconazole |
| B1-610 | N(H)CO₂Et | O | O | CO₂H | — | — | 0 | O | voriconazole |
| B1-611 | Me | O | O | CO₂H | — | — | 0 | O | fluconazole |
| B1-612 | cyclohexyl | O | O | CO₂H | — | — | 0 | O | fluconazole |
| B1-613 | t-Bu | O | O | CO₂H | — | — | 0 | O | fluconazole |
| B1-614 | CF₃CH₂ | O | O | CO₂H | — | — | 0 | O | fluconazole |
| B1-615 | allyl | O | O | CO₂H | — | — | 0 | O | fluconazole |
| B1-616 | 4-(NO₂)Ph | O | O | CO₂H | — | — | 0 | O | fluconazole |
| B1-617 | PhS | O | O | CO₂H | — | — | 0 | O | fluconazole |
| B1-618 | N(Me)Ph | O | O | CO₂H | — | — | 0 | O | fluconazole |
| B1-619 | N(H)OMe | O | O | CO₂H | — | — | 0 | O | fluconazole |
| B1-620 | N(H)CO₂Et | O | O | CO₂H | — | — | 0 | O | fluconazole |
| B1-621 | Me | O | O | CO₂H | — | — | 0 | O | genaconazole |
| B1-622 | cyclohexyl | O | O | CO₂H | — | — | 0 | O | genaconazole |
| B1-623 | t-Bu | O | O | CO₂H | — | — | 0 | O | genaconazole |
| B1-624 | CF₃CH₂ | O | O | CO₂H | — | — | 0 | O | genaconazole |
| B1-625 | allyl | O | O | CO₂H | — | — | 0 | O | genaconazole |
| B1-626 | 4-(NO₂)Ph | O | O | CO₂H | — | — | 0 | O | genaconazole |
| B1-627 | PhS | O | O | CO₂H | — | — | 0 | O | genaconazole |
| B1-628 | N(Me)Ph | O | O | CO₂H | — | — | 0 | O | genaconazole |
| B1-629 | N(H)OMe | O | O | CO₂H | — | — | 0 | O | genaconazole |
| B1-630 | N(H)CO₂Et | O | O | CO₂H | — | — | 0 | O | genaconazole |
| B1-631 | Me | O | O | CO₂H | — | — | 0 | N | aliconazole |
| B1-632 | cyclohexyl | O | O | CO₂H | — | — | 0 | N | aliconazole |
| B1-633 | t-Bu | O | O | CO₂H | — | — | 0 | N | aliconazole |
| B1-634 | CF₃CH₂ | O | O | CO₂H | — | — | 0 | N | aliconazole |
| B1-635 | allyl | O | O | CO₂H | — | — | 0 | N | aliconazole |
| B1-636 | 4-(NO₂)Ph | O | O | CO₂H | — | — | 0 | N | aliconazole |
| B1-637 | PhS | O | O | CO₂H | — | — | 0 | N | aliconazole |
| B1-638 | N(Me)Ph | O | O | CO₂H | — | — | 0 | N | aliconazole |
| B1-639 | N(H)OMe | O | O | CO₂H | — | — | 0 | N | aliconazole |
| B1-640 | N(H)CO₂Et | O | O | CO₂H | — | — | 0 | N | aliconazole |
| B1-641 | Me | O | O | CO₂H | — | — | 0 | N | becliconazole |
| B1-642 | cyclohexyl | O | O | CO₂H | — | — | 0 | N | beciconazole |
| B1-643 | t-Bu | O | O | CO₂H | — | — | 0 | N | beciconazole |
| B1-644 | CF₃CH₂ | O | O | CO₂H | — | — | 0 | N | beciconazole |
| B1-645 | allyl | O | O | CO₂H | — | — | 0 | N | becliconazole |
| B1-646 | 4-(NO₂)Ph | O | O | CO₂H | — | — | 0 | N | becliconazole |
| B1-647 | PhS | O | O | CO₂H | — | — | 0 | N | beciconazole |
| B1-648 | N(Me)Ph | O | O | CO₂H | — | — | 0 | N | becliconazole |
| B1-649 | N(H)OMe | O | O | CO₂H | — | — | 0 | N | becliconazole |
| B1-650 | N(H)CO₂Et | O | O | CO₂H | — | — | 0 | N | beciconazole |
| B1-651 | Me | O | O | CO₂H | — | — | 0 | N | brolaconazole |
| B1-652 | cyclohexyl | O | O | CO₂H | — | — | 0 | N | brolaconazole |
| B1-653 | t-Bu | O | O | CO₂H | — | — | 0 | N | brolaconazole |
| B1-654 | CF₃CH₂ | O | O | CO₂H | — | — | 0 | N | brolaconazole |
| B1-655 | aIlyl | O | O | CO₂H | — | — | 0 | N | brolaconazole |
| B1-656 | 4-(NO₂)Ph | O | O | CO₂H | — | — | 0 | N | brolaconazole |
| B1-657 | PhS | O | O | CO₂H | — | — | 0 | N | brolaconazole |
| B1-658 | N(Me)Ph | O | O | CO₂H | — | — | 0 | N | brolaconazole |
| B1-659 | N(H)OMe | O | O | CO₂H | — | — | 0 | N | brolaconazole |
| B1-660 | N(H)CO₂Et | O | O | CO₂H | — | — | 0 | N | brolaconazole |
| B1-661 | Me | O | O | CO₂H | — | — | 0 | N | butaconazole |
| B1-662 | cyclohexyl | O | O | CO₂H | — | — | 0 | N | butaconazole |
| B1-663 | t-Bu | O | O | CO₂H | — | — | 0 | N | butaconazole |
| B1-664 | CF₃CH₂ | O | O | CO₂H | — | — | 0 | N | butaconazole |
| B1-665 | allyl | O | O | CO₂H | — | — | 0 | N | butaconazole |
| B1-666 | 4-(NO₂)Ph | O | O | CO₂H | — | — | 0 | N | butaconazole |
| B1-667 | PhS | O | O | CO₂H | — | — | 0 | N | butaconazole |
| B1-668 | N(Me)Ph | O | O | CO₂H | — | — | 0 | N | butaconazole |
| B1-669 | N(H)OMe | O | O | CO₂H | — | — | 0 | N | butaconazole |
| B1-670 | N(H)CO₂Et | O | O | CO₂H | — | — | 0 | N | butaconazole |
| B1-671 | Me | O | O | CO₂H | — | — | 0 | N | clotrimazole |
| B1-672 | cyclohexyl | O | O | CO₂H | — | — | 0 | N | clotriinazole |
| B1-673 | t-Bu | O | O | CO₂H | — | — | 0 | N | clotrimazole |
| B1-674 | CF₃CH₂ | O | O | CO₂H | — | — | 0 | N | clotrimazole |
| B1-675 | allyl | O | O | CO₂H | — | — | 0 | N | clotrimazole |
| B1-676 | 4-(NO₂)Ph | O | O | CO₂H | — | — | 0 | N | clotrimazole |
| B1-677 | PhS | O | O | CO₂H | — | — | 0 | N | clotrimazole |
| B1-678 | N(Me)Ph | O | O | CO₂H | — | — | 0 | N | clotrimazole |
| B1-679 | N(H)OMe | O | O | CO₂H | — | — | 0 | N | clotrimazole |
| B1-680 | N(H)CO₂Et | O | O | CO₂H | — | — | 0 | N | clotrimazole |
| B1-681 | Me | O | O | CO₂H | — | — | 0 | N | croconazole |
| B1-682 | cyclohexyl | O | O | CO₂H | — | — | 0 | N | croconazole |
| B1-683 | t-Bu | O | O | CO₂H | — | — | 0 | N | croconazole |
| B1-684 | CF₃CH₂ | O | O | CO₂H | — | — | 0 | N | croconazole |
| B1-685 | allyl | O | O | CO₂H | — | — | 0 | N | croconazole |
| B1-686 | 4-(NO₂)Ph | O | O | CO₂H | — | — | 0 | N | croconazole |
| B1-687 | PhS | O | O | CO₂H | — | — | 0 | N | croconazole |
| B1-688 | N(Me)Ph | O | O | CO₂H | — | — | 0 | N | croconazole |
| B1-689 | N(H)OMe | O | O | CO₂H | — | — | 0 | N | croconazole |
| B1-690 | N(H)CO₂Et | O | O | CO₂H | — | — | 0 | N | croconazole |
| B1-691 | Me | O | O | CO₂H | — | — | 0 | N | econazole |
| B1-692 | cyclohexyl | O | O | CO₂H | — | — | 0 | N | econazole |
| B1-693 | t-Bu | O | O | CO₂H | — | — | 0 | N | econazole |
| B1-694 | CF₃CH₂ | O | O | CO₂H | — | — | 0 | N | econazole |
| B1-695 | allyl | O | O | CO₂H | — | — | 0 | N | econazole |
| B1-696 | 4-(NO₂)Ph | O | O | CO₂H | — | — | 0 | N | econazole |
| B1-697 | PhS | O | O | CO₂H | — | — | 0 | N | econazole |
| B1-698 | N(Me)Ph | O | O | CO₂H | — | — | 0 | N | econazole |
| B1-699 | N(H)OMe | O | O | CO₂H | — | — | 0 | N | econazole |
| B1-700 | N(H)CO₂Et | O | O | CO₂H | — | — | 0 | N | econazole |
| B1-701 | Me | O | O | CO₂H | — | — | 0 | N | democonazole |
| B1-702 | cyclohexyl | O | O | CO₂H | — | — | 0 | N | democonazole |
| B1-703 | t-Bu | O | O | CO₂H | — | — | 0 | N | democonazole |
| B1-704 | CF₃CH₂ | O | O | CO₂H | — | — | 0 | N | democonazole |
| B1-705 | aHyl | O | O | CO₂H | — | — | 0 | N | democonazole |
| B1-706 | 4-(NO₂)Ph | O | O | CO₂H | — | — | 0 | N | democonazole |
| B1-707 | PhS | O | O | CO₂H | — | — | 0 | N | democonazole |
| B1-708 | N(Me)Ph | O | O | CO₂H | — | — | 0 | N | democonazole |
| B1-709 | N(H)OMe | O | O | CO₂H | — | — | 0 | N | democonazole |
| B1-710 | N(H)CO₂Et | O | O | CO₂H | — | — | 0 | N | democonazole |
| B1-711 | Me | O | O | CO₂H | — | — | 0 | N | doconazole |
| B1-712 | cyclohexyl | O | O | CO₂H | — | — | 0 | N | doconazole |
| B1-713 | t-Bu | O | O | CO₂H | — | — | 0 | N | doconazole |
| B1-714 | CF₃CH₂ | O | O | CO₂H | — | — | 0 | N | doconazole |
| B1-715 | allyl | O | O | CO₂H | — | — | 0 | N | doconazole |
| B1-716 | 4-(NO₂)Ph | O | O | CO₂H | — | — | 0 | N | doconazole |
| B1-717 | PhS | O | O | CO₂H | — | — | 0 | N | doconazole |
| B1-718 | N(Me)Ph | O | O | CO₂H | — | — | 0 | N | doconazole |
| B1-719 | N(H)OMe | O | O | CO₂H | — | — | 0 | N | doconazale |
| B1-720 | N(H)CO₂Et | O | O | CO₂H | — | — | 0 | N | doconazole |
| B1-721 | Me | O | O | CO₂H | — | — | 0 | N | fenticonazole |
| B1-722 | cyclohexyl | O | O | CO₂H | — | — | 0 | N | fenticonazole |
| B1-723 | t-Bu | O | O | CO₂H | — | — | 0 | N | fenticonazole |
| B1-724 | CF₃CH₂ | O | O | CO₂H | — | — | 0 | N | fenticonazole |
| B1-725 | allyl | O | O | CO₂H | — | — | 0 | N | fenticonazole |
| B1-726 | 4-(NO₂)Ph | O | O | CO₂H | — | — | 0 | N | fenticonazo1e |
| B1-727 | PhS | O | O | CO₂H | — | — | 0 | N | fenticonazole |
| B1-728 | N(Me)Ph | O | O | CO₂H | — | — | 0 | N | fenticonazole |
| B1-729 | N(H)OMe | O | O | CO₂H | — | — | 0 | N | fenticonazole |
| B1-730 | N(H)CO₂Et | O | O | CO₂H | — | — | 0 | N | fenticonazole |
| B1-731 | Me | O | O | CO₂H | — | — | 0 | N | eberconazole |
| B1-732 | cyclohexyl | O | O | CO₂H | — | — | 0 | N | eberconazole |
| B1-733 | t-Bu | O | O | CO₂H | — | — | 0 | N | eberconazole |
| B1-734 | CF₃CH₂ | O | O | CO₂H | — | — | 0 | N | eberconazole |
| B1-735 | allyl | O | O | CO₂H | — | — | 0 | N | eberconazole |
| B1-736 | 4-(NO₂)Ph | O | O | CO₂H | — | — | 0 | N | eberconazole |
| B1-737 | PhS | O | O | CO₂H | — | — | 0 | N | eberconazole |
| B1-738 | N(Me)Ph | O | O | CO₂H | — | — | 0 | N | eberconazole |
| B1-739 | N(H)OMe | O | O | CO₂H | — | — | 0 | N | eberconazole |
| B1-740 | N(H)CO₂Et | O | O | CO₂H | — | — | 0 | N | eberconazole |
| B1-741 | Me | O | O | CO₂H | — | — | 0 | N | isoconazole |
| B1-742 | cyclohexyl | O | O | CO₂H | — | — | 0 | N | isoconazole |
| B1-743 | t-Bu | O | O | CO₂H | — | — | 0 | N | isoconazole |
| B1-744 | CF₃CH₂ | O | O | CO₂H | — | — | 0 | N | isoconazole |
| B1-745 | allyl | O | O | CO₂H | — | — | 0 | N | isoconazole |
| B1-746 | 4-(NO₂)Ph | O | O | CO₂H | — | — | 0 | N | isoconazole |
| B1-747 | PhS | O | O | CO₂H | — | — | 0 | N | isoconazole |
| B1-748 | N(Me)Ph | O | O | CO₂H | — | — | 0 | N | isoconazole |
| B1-749 | N(H)OMe | O | O | CO₂H | — | — | 0 | N | isoconazole |
| B1-750 | N(H)CO₂Et | O | O | CO₂H | — | — | 0 | N | isoconazole |
| B1-751 | Me | O | O | CO₂H | — | — | 0 | N | miconazole |
| B1-752 | cyclohexyl | O | O | CO₂H | — | — | 0 | N | miconazole |
| B1-753 | t-Bu | O | O | CO₂H | — | — | 0 | N | miconazole |
| B1-754 | CF₃CH₂ | O | O | 0021-I | — | — | 0 | N | miconazole |
| B1-755 | allyl | O | O | CO₂H | — | — | 0 | N | miconazole |
| B1-756 | 4-(NO₂)Ph | O | O | CO₂H | — | — | 0 | N | miconazole |
| B1-757 | PhS | O | O | CO₂H | — | — | 0 | N | miconazole |
| B1-758 | N(Me)Ph | O | O | CO₂H | — | — | 0 | N | miconazole |
| B1-759 | N(H)OMe | O | O | CO₂H | — | — | 0 | N | miconazole |
| B1-760 | N(H)CO₂Et | O | O | CO₂H | — | — | 0 | N | miconazole |
| B1-761 | Me | O | O | CO₂H | — | — | 0 | N | neticonazole |
| B1-762 | cyclohexyl | O | O | CO₂H | — | — | 0 | N | neticonazole |
| B1-763 | t-Bu | O | O | CO₂H | — | — | 0 | N | neticonazole |
| B1-764 | CF₃CH₂ | O | O | CO₂H | — | — | 0 | N | neticonazole |
| B1-765 | allyl | O | O | CO₂H | — | — | 0 | N | neticonazole |
| B1-766 | 4-(NO₂)Ph | O | O | CO₂H | — | — | 0 | N | neticonazole |
| B1-767 | PhS | O | O | CO₂H | — | — | 0 | N | neticonazole |
| B1-768 | N(Me)Ph | O | O | CO₂H | — | — | 0 | N | neticonazole |
| B1-769 | N(H)OMe | O | O | CO₂H | — | — | 0 | N | neticonazole |
| B1-770 | N(H)CO₂Et | O | O | CO₂H | — | — | 0 | N | neticonazole |
| B1-771 | Me | O | O | CO₂H | — | — | 0 | N | omoconazole |
| B1-772 | cyclohexyl | O | O | CO₂H | — | — | 0 | N | omoconazole |
| B1-773 | t-Bu | O | O | CO₂H | — | — | 0 | N | omoconazole |
| B1-774 | CF₃CH₂ | O | O | CO₂H | — | — | 0 | N | omoconazole |
| B1-775 | allyl | O | O | CO₂H | — | — | 0 | N | omoconazole |
| B1-776 | 4-(NO₂)Ph | O | O | CO₂H | — | — | 0 | N | omoconazole |
| B1-777 | PhS | 0 | O | CO₂H | — | — | 0 | N | omoconazole |
| B1-778 | N(Me)Ph | O | O | CO₂H | — | — | 0 | N | omoconazole |
| B1-779 | N(H)OMe | O | O | CO₂H | — | — | 0 | N | omoconazole |
| B1-780 | N(H)CO₂Et | O | O | CO₂H | — | — | 0 | N | omoconazole |
| B1-781 | Me | O | O | CO₂H | — | — | 0 | N | orconazole |
| B1-782 | cyclohexyl | O | O | CO₂H | — | — | 0 | N | orconazole |
| B1-783 | t-Bu | O | O | CO₂H | — | — | 0 | N | orconazole |
| B1-784 | CF₃CH₂ | O | O | CO₂H | — | — | 0 | N | orconazole |
| B1-785 | allyl | O | O | CO₂H | — | — | 0 | N | orconazole |
| B1-786 | 4-(NO₂)Ph | O | O | CO₂H | — | — | 0 | N | orconazole |
| B1-787 | PhS | O | O | CO₂H | — | — | 0 | N | orconazole |
| B1-788 | N(Me)Ph | O | O | CO₂H | — | — | 0 | N | orconazole |
| B1-789 | N(H)OMe | O | O | CO₂H | — | — | 0 | N | orconazole |
| B1-790 | N(H)CO₂Et | O | O | CO₂H | — | — | 0 | N | orconazole |
| B1-791 | Me | O | O | CO₂H | — | — | 0 | N | oxiconazole |
| B1-792 | cyclohexyl | O | O | CO₂H | — | — | 0 | N | oxiconazole |
| B1-793 | t-Bu | O | O | CO₂H | — | — | 0 | N | oxiconazole |
| B1-794 | CF₃CH₂ | O | O | CO₂H | — | — | 0 | N | oxiconazole |
| B1-795 | allyl | O | O | CO₂H | — | — | 0 | N | oxiconazole |
| B1-796 | 4-(NO₂)Ph | O | O | CO₂H | — | — | 0 | N | oxiconazole |
| B1-797 | PhS | O | O | CO₂H | — | — | 0 | N | oxiconazole |
| B1-798 | N(Me)Ph | O | O | CO₂H | — | — | 0 | N | oxiconazole |
| B1-799 | N(H)OMe | O | O | CO₂H | — | — | 0 | N | oxiconazole |
| B1-800 | N(H)CO₂Et | O | O | CO₂H | — | — | 0 | N | oxiconazole |
| B1-801 | Me | O | O | CO₂H | — | — | 0 | N | parconazole |
| B1-802 | cyclohexyl | O | O | CO₂H | — | — | 0 | N | parconazole |
| B1-803 | t-Bu | O | O | CO₂H | — | — | 0 | N | parconazole |
| B1-804 | CF₃CH₂ | O | O | CO₂H | — | — | 0 | N | parconazole |
| B1-805 | allyl | O | O | CO₂H | — | — | 0 | N | parconazole |
| B1-806 | 4-(NO₂)Ph | O | O | CO₂H | — | — | 0 | N | parconazole |
| B1-807 | PhS | O | O | CO₂H | — | — | 0 | N | parconazole |
| B1-808 | N(Me)Ph | O | O | CO₂H | — | — | 0 | N | parconazole |
| B1-809 | N(H)OMe | O | O | CO₂H | — | — | 0 | N | parconazole |
| B1-810 | N(H)CO₂Et | O | O | CO₂H | — | — | 0 | N | parconazole |
| B1-811 | Me | O | O | CO₂H | — | — | 0 | N | ravuconazole |
| B1-812 | cyclohexyl | O | O | CO₂H | — | — | 0 | N | ravuconazole |
| B1-813 | t-Bu | O | O | CO₂H | — | — | 0 | N | ravuconazole |
| B1-814 | CF₃CH₂ | O | O | CO₂H | — | — | 0 | N | ravuconazole |
| B1-815 | allyl | O | O | CO₂H | — | — | 0 | N | ravuconazole |
| B1-816 | 4-(NO₂)Ph | O | O | CO₂H | — | — | 0 | N | ravuconazole |
| B1-817 | PhS | O | O | CO₂H | — | — | 0 | N | ravuconazole |
| B1-818 | N(Me)Ph | O | O | CO₂H | — | — | 0 | N | ravuconazole |
| B1-819 | N(H)OMe | O | O | CO₂H | — | — | 0 | N | ravuconazole |
| B1-820 | N(H)CO₂Et | O | O | CO₂H | — | — | 0 | N | ravuconazole |
| B1-821 | Me | O | O | CO₂H | — | — | 0 | N | sertaconazole |
| B1-822 | cyclohexyl | O | O | CO₂H | — | — | 0 | N | sertaconazole |
| B1-823 | t-Bu | O | O | CO₂H | — | — | 0 | N | sertaconazole |
| B1-824 | CF₃CH₂ | O | O | CO₂H | — | — | 0 | N | sertaconazole |
| B1-825 | allyl | O | O | CO₂H | — | — | 0 | N | sertaconazole |
| B1-826 | 4-(NO₂)Ph | O | O | CO₂H | — | — | 0 | N | sertaconazole |
| B1-827 | PhS | O | O | CO₂H | — | — | 0 | N | sertaconazole |
| B1-828 | N(Me)Ph | O | O | CO₂H | — | — | 0 | N | sertaconazole |
| B1-829 | N(H)OMe | O | O | CO₂H | — | — | 0 | N | sertaconazole |
| B1-830 | N(H)CO₂Et | O | O | CO₂H | — | — | 0 | N | sertaconazole |
| B1-831 | Me | O | O | CO₂H | — | — | 0 | N | sulconazole |
| B1-832 | cyclohexyl | O | O | CO₂H | — | — | 0 | N | sulconazole |
| B1-833 | t-Bu | O | O | CO₂H | — | — | 0 | N | sulconazole |
| B1-834 | CF₃CH₂ | O | O | CO₂H | — | — | 0 | N | sulconazole |
| B1-835 | allyl | O | O | CO₂H | — | — | 0 | N | sulconazole |
| B1-836 | 4-(NO₂)Ph | O | O | CO₂H | — | — | 0 | N | sulconazole |
| B1-837 | PhS | O | O | CO₂H | — | — | 0 | N | sulconazole |
| B1-838 | N(Me)Ph | O | O | CO₂H | — | — | 0 | N | sulconazole |
| B1-839 | N(H)OMe | O | O | CO₂H | — | — | 0 | N | sulconazole |
| B1-840 | N(H)CO₂Et | O | O | CO₂H | — | — | 0 | N | sulconazole |
| B1-841 | Me | O | O | CO₂H | — | — | 0 | N | tioconazole |
| B1-842 | cyclohexyl | O | O | CO₂H | — | — | 0 | N | tioconazole |
| B1-843 | t-Bu | O | O | CO₂H | — | — | 0 | N | tioconazole |
| B1-844 | CF₃CH₂ | O | O | CO₂H | — | — | 0 | N | tioconazole |
| B1-845 | allyl | O | O | CO₂H | — | — | 0 | N | tioconazole |
| B1-846 | 4-(NO₂)Ph | O | O | CO₂H | — | — | 0 | N | tioconazole |
| B1-847 | PhS | O | O | CO₂H | — | — | 0 | N | tioconazole |
| B1-848 | N(Me)Ph | O | O | CO₂H | — | — | 0 | N | tioconazole |
| B1-849 | N(H)OMe | O | O | CO₂H | — | — | 0 | N | tioconazole |
| B1-850 | N(H)CO₂Et | O | O | CO₂H | — | — | 0 | N | tioconazole |
| B1-851 | Me | O | O | CO₂H | — | — | 0 | N | valconazole |
| B1-852 | cyclohexyl | O | O | CO₂H | — | — | 0 | N | valconazole |
| B1-853 | t-Bu | O | O | CO₂H | — | — | 0 | N | valconazole |
| B1-854 | CF₃CH₂ | O | O | CO₂H | — | — | 0 | N | vakonazole |
| B1-855 | allyl | O | O | CO₂H | — | — | 0 | N | valconazole |
| B1-856 | 4-(NO₂)Ph | O | O | CO₂H | — | — | 0 | N | valconazole |
| B1-857 | PhS | O | O | CO₂H | — | — | 0 | N | valconazale |
| B1-858 | N(Me)Ph | O | O | CO₂H | — | — | 0 | N | valconazole |
| B1-859 | N(H)OMe | O | O | CO₂H | — | — | 0 | N | valconazole |
| B1-860 | N(H)CO₂Et | O | O | CO₂H | — | — | 0 | N | valconazole |
| B1-861 | Me | O | O | CO₂H | — | — | 0 | N | zinoconazole |
| B1-862 | cyclohexyl | O | O | CO₂H | — | — | 0 | N | zinoconazole |
| B1-863 | t-Bu | O | O | CO₂H | — | — | 0 | N | zinoconazole |
| B1-864 | CF₃CH₂ | O | O | CO₂H | — | — | 0 | N | zinoconazole |
| B1-865 | allyl | O | O | CO₂H | — | — | 0 | N | zinoconazole |
| B1-866 | 4-(NO₂)Ph | O | O | CO₂H | — | — | 0 | N | zinoconazole |
| B1-867 | PhS | O | O | CO₂H | — | — | 0 | N | zinoconazole |
| B1-868 | N(Me)Ph | O | O | CO₂H | — | — | 0 | N | zinoconazole |
| B1-869 | N(H)OMe | O | O | CO₂H | — | — | 0 | N | zinoconazole |
| B1-870 | N(H)CO₂Et | O | O | CO₂H | — | — | 0 | N | zinoconazole |
| B1-871 | Me | O | O | CO₂H | O | O | 1 | C | cloxacillin |
| B1-872 | cyclohexyl | O | O | CO₂H | O | O | 1 | C | cloxacillin |
| B1-873 | t-Bu | O | O | CO₂H | O | O | 1 | C | cloxacillin |
| B1-874 | CF₃CH₂ | O | O | CO₂H | O | O | 1 | C | cloxacillin |
| B1-875 | alLyl | O | O | CO₂H | O | O | 1 | C | cloxacillin |
| B1-876 | 4-(NO₂)Ph | O | O | CO₂H | O | O | 1 | C | cloxacillin |
| B1-877 | PhS | O | O | CO₂H | O | O | 1 | C | cloxacillin |
| B1-878 | N(Me)Ph | O | O | CO₂H | O | O | 1 | C | cloxacillin |
| B1-879 | N(H)OMe | O | O | CO₂H | O | O | 1 | C | cloxacillin |
| B1-880 | N(H)CO₂Et | O | O | CO₂H | O | O | 1 | C | cloxacillin |
| B1-881 | Me | O | O | CO₂H | O | O | 1 | C | valproic acid |
| B1-882 | cyclohexyl | O | O | CO₂H | O | O | 1 | C | valproic acid |
| B1-883 | t-Bu | O | O | CO₂H | O | O | 1 | C | valproic acid |
| B1-884 | CF₃CH₂ | O | O | CO₂H | O | O | 1 | C | valproic acid |
| B1-885 | allyl | O | O | CO₂H | O | O | 1 | C | valproic acid |
| B1-886 | 4-(NO₂)Ph | O | O | CO₂H | O | O | 1 | C | valproic acid |
| B1-887 | PhS | O | O | CO₂H | O | O | 1 | C | valproic acid |
| B1-888 | N(Me)Ph | O | O | CO₂H | O | O | 1 | C | valproic acid |
| B1-889 | N(H)OMe | O | O | CO₂H | O | O | 1 | C | valproic acid |
| B1-890 | N(H)CO₂Et | O | O | CO₂H | O | O | 1 | C | valproic acid |
| B1-891 | Me | O | O | CO₂H | O | O | 1 | C | retinoic acid |
| B1-892 | cyclohexyl | O | O | CO₂H | O | O | 1 | C | retinoic acid |
| B1-893 | t-Bu | O | O | CO₂H | O | O | 1 | C | retinoic acid |
| B1-894 | CF₃CH₂ | O | O | CO₂H | O | O | 1 | C | retinoic acid |
| B1-895 | allyl | O | O | CO₂H | O | O | 1 | C | retinoic acid |
| B1-896 | 4-(NO₂)Ph | O | O | CO₂H | O | O | 1 | C | retinoic acid |
| B1-897 | PhS | O | O | CO₂H | O | O | 1 | C | retinoic acid |
| B1-898 | N(Me)Ph | O | O | CO₂H | O | O | 1 | C | retinoic acid |
| B1-899 | N(H)OMe | O | O | CO₂H | O | O | 1 | C | retinoic acid |
| B1-900 | N(H)CO₂Et | O | O | CO₂H | O | O | 1 | C | retinoic acid |
| B1-901 | Me | O | O | CO₂H | O | O | 1 | C | oxaprozin |
| B1-902 | cyclohexyl | O | O | CO₂H | O | O | 1 | C | oxaprozin |
| B1-903 | t-Bu | O | O | CO₂H | O | O | 1 | C | oxaprozin |
| B1-904 | CF₃CH₂ | O | O | CO₂H | O | O | 1 | C | oxaprozrn |
| B1-905 | allyl | O | O | CO₂H | O | O | 1 | C | oxaprozin |
| B1-906 | 4-(NO₂)Ph | O | O | CO₂H | O | O | 1 | C | oxaprozin |
| B1-907 | PhS | O | O | CO₂H | O | O | 1 | C | oxaprozin |
| B1-908 | N(Me)Ph | O | O | CO₂H | O | O | 1 | C | oxaprozin |
| B1-909 | N(H)OMe | O | O | CO₂H | O | O | 1 | C | oxaprozin |
| B1-910 | N(H)CO₂Et | O | O | CO₂H | O | O | 1 | C | oxaprozin |
| B1-911 | Me | O | O | CO₂H | O | O | 1 | C | naproxen |
| B1-912 | cyclohexyl | O | O | CO₂H | O | O | 1 | C | naproxen |
| B1-913 | t-Bu | O | O | CO₂H | O | O | 1 | C | naproxen |
| B1-914 | CF₃CH₂ | O | O | CO₂H | O | O | 1 | C | naproxen |
| B1-915 | allyl | O | O | CO₂H | O | O | 1 | C | naproxen |
| B1-916 | 4-(NO₂)Ph | O | O | CO₂H | O | O | 1 | C | naproxen |
| B1-917 | PhS | O | O | CO₂H | O | O | 1 | C | naproxen |
| B1-918 | N(Me)Ph | O | O | CO₂H | O | O | 1 | C | naproxen |
| B1-919 | N(H)OMe | O | O | CO₂H | O | O | 1 | C | naproxen |
| B1-920 | N(H)CO₂Et | O | O | CO₂H | O | O | 1 | C | naproxen |
| B1-921 | Me | O | O | CO₂H | O | O | 1 | C | monopril |
| B1-922 | cyclohexyl | O | O | CO₂H | O | O | 1 | C | monopril |
| B1-923 | t-Bu | O | O | CO₂H | O | O | 1 | C | monopril |
| B1-924 | CF₃CH₂ | O | O | CO₂H | O | O | 1 | C | monopril |
| B1-925 | allyl | O | O | CO₂H | O | O | 1 | C | monopril |
| B1-926 | 4-(NO₂)Ph | O | O | CO₂H | O | O | 1 | C | monopril |
| B1-927 | PhS | O | O | CO₂H | O | O | 1 | C | monopril |
| B1-928 | N(Me)Ph | O | O | CO₂H | O | O | 1 | C | monopril |
| B1-929 | N(H)OMe | O | O | CO₂H | O | O | 1 | C | monopril |
| B1-930 | N(H)CO₂Et | O | O | CO₂H | O | O | 1 | C | monopril |
| B1-931 | Me | O | O | CO₂H | O | O | 1 | C | ketorolac |
| B1-932 | cyclohexyl | O | O | CO₂H | O | O | 1 | C | ketorolac |
| B1-933 | t-Bu | O | O | CO₂H | O | O | 1 | C | ketorolac |
| B1-934 | CF₃CH₂ | O | O | CO₂H | O | O | 1 | C | ketorolac |
| B1-935 | allyl | O | O | CO₂H | O | O | 1 | C | ketorolac |
| B1-936 | 4-(NO₂)Ph | O | O | CO₂H | O | O | 1 | C | ketorolac |
| B1-937 | PhS | O | O | CO₂H | O | O | 1 | C | ketorolac |
| B1-938 | N(Me)Ph | O | O | CO₂H | O | O | 1 | C | ketorolac |
| B1-939 | N(H)OMe | O | O | CO₂H | O | O | 1 | C | ketorolac |
| B1-940 | N(H)CO₂Et | O | O | CO₂H | O | O | 1 | C | ketorolac |
| B1-941 | Me | O | O | CO₂H | O | O | 1 | C | ketoprofen |
| B1-942 | cyclohexyl | O | O | CO₂H | O | O | 1 | C | ketoprofen |
| B1-943 | t-Bu | O | O | CO₂H | O | O | 1 | C | ketoprofen |
| B1-944 | CF₃CH₂ | O | O | CO₂H | O | O | 1 | C | ketoprofen |
| B1-945 | allyl | O | O | CO₂H | O | O | 1 | C | ketoprofen |
| B1-946 | 4-(NO₂)Ph | O | O | CO₂H | O | O | 1 | C | ketoprofen |
| B1-947 | PhS | O | O | CO₂H | O | O | 1 | C | ketoprofen |
| B1-948 | N(Me)Ph | O | O | CO₂H | O | O | 1 | C | ketoprofen |
| B1-949 | N(H)OMe | O | O | CO₂H | O | O | 1 | C | ketoprofen |
| B1-950 | N(H)CO₂Et | O | O | CO₂H | O | O | 1 | C | ketoprofen |
| B1-951 | Me | O | O | CO₂H | O | O | 1 | C | indomethacin |
| B1-952 | cyclohexyl | O | O | CO₂H | O | O | 1 | C | indomethacin |
| B1-953 | t-Bu | O | O | CO₂H | O | O | 1 | C | indomethacin |
| B1-954 | CF₃CH₂ | O | O | CO₂H | O | O | 1 | C | indomethacin |
| B1-955 | allyl | O | O | CO₂H | O | O | 1 | C | indomethacin |
| B1-956 | 4-(NO₂)Ph | O | O | CO₂H | O | O | 1 | C | indomethacin |
| B1-957 | PhS | O | O | CO₂H | O | O | 1 | C | indomethacin |
| B1-958 | N(Me)Ph | O | O | CO₂H | O | O | 1 | C | indomethacin |
| B1-959 | N(H)OMe | O | O | CO₂H | O | O | 1 | C | indomethacin |
| B1-960 | N(H)CO₂Et | O | O | CO₂H | O | O | 1 | C | indomethacin |
| B1-961 | Me | O | O | CO₂H | O | O | 1 | C | ibuprofen |
| B1-962 | cyclohexyl | O | O | CO₂H | O | O | 1 | C | ibuprofen |
| B1-963 | t-Bu | O | O | CO₂H | O | O | 1 | C | ibuprofen |
| B1-964 | CF₃CH₂ | O | O | CO₂H | O | O | 1 | C | ibuprofen |
| B1-965 | allyl | O | O | CO₂H | O | O | 1 | C | ibuprofen |
| B1-966 | 4-(NO₂)Ph | O | O | CO₂H | O | O | 1 | C | ibuprofen |
| B1-967 | PhS | O | O | CO₂H | O | O | 1 | C | ibuprofen |
| B1-968 | N(Me)Ph | O | O | CO₂H | O | O | 1 | C | ibuprofen |
| B1-969 | N(H)OMe | O | O | CO₂H | O | O | 1 | C | ibuprofen |
| B1-970 | N(H)CO₂Et | O | O | CO₂H | O | O | 1 | C | ibuprofen |
| B1-971 | Me | O | O | CO₂H | O | O | 1 | C | gemfibrozil |
| B1-972 | cyclohexyl | O | O | CO₂H | O | O | 1 | C | gemfibrozil |
| B1-973 | t-Bu | O | O | CO₂H | O | O | 1 | C | gemfibrozil |
| B1-974 | CF₃CH₂ | O | O | CO₂H | O | O | 1 | C | gemfibrozil |
| B1-975 | allyl | O | O | CO₂H | O | O | 1 | C | gemfibrozil |
| B1-976 | 4-(NO₂)Ph | O | O | CO₂H | O | O | 1 | C | gemfibrozil |
| B1-977 | PhS | O | O | CO₂H | O | O | 1 | C | gemfibrozil |
| B1-978 | N(Me)Ph | O | O | CO₂H | O | O | 1 | C | gemfibrozil |
| B1-979 | N(H)OMe | O | O | CO₂H | O | O | 1 | C | gemfibrozil |
| B1-980 | N(H)CO₂Et | O | O | CO₂H | O | O | 1 | C | gemfibrozil |
| B1-981 | Me | O | O | CO₂H | O | O | 1 | C | flubiprofen |
| B1-982 | cyclohexyl | O | O | CO₂H | O | O | 1 | C | flurbiprofen |
| B1-983 | t-Bu | O | O | CO₂H | O | O | 1 | C | flurbiprofen |
| B1-984 | CF₃CH₂ | O | O | CO₂H | O | O | 1 | C | flurbiprofen |
| B1-985 | allyl | O | O | CO₂H | O | O | 1 | C | flurbiprofen |
| B1-986 | 4-(NO₂)Ph | O | O | CO₂H | O | O | 1 | C | flurbiprofen |
| B1-987 | PhS | O | O | CO₂H | O | O | 1 | C | flurbiprofen |
| B1-988 | N(Me)Ph | O | O | CO₂H | O | O | 1 | C | flurbiprofen |
| B1-989 | N(H)OMe | O | O | CO₂H | O | O | 1 | C | flurbiprofen |
| B1-990 | N(H)CO₂Et | O | O | CO₂H | O | O | 1 | C | flurbiprofen |
| B1-991 | Me | O | O | CO₂H | O | O | 1 | C | arthrocine |
| B1-992 | cyclohexyl | O | O | CO₂H | O | O | 1 | C | arthrocine |
| B1-993 | t-Bu | O | O | CO₂H | O | O | 1 | C | arthrocine |
| B1-994 | CF₃CH₂ | O | O | CO₂H | O | O | 1 | C | arthrocine |
| B1-995 | allyl | O | O | CO₂H | O | O | 1 | C | arthrocine |
| B1-996 | 4-(NO₂)Ph | O | O | CO₂H | O | O | 1 | C | arthrocine |
| B1-997 | PhS | O | O | CO₂H | O | O | 1 | C | arthrocine |
| B1-998 | N(Me)Ph | O | O | CO₂H | O | O | 1 | C | arthrocine |
| B1-999 | N(H)OMe | O | O | CO₂H | O | O | 1 | C | arthrocine |
| B1-1000 | N(H)CO₂Et | O | O | CO₂H | O | O | 1 | C | arthrocine |
| B1-1001 | Me | O | O | CO₂H | O | O | 1 | C | adapalene |
| B1-1002 | cyclohexyl | O | O | CO₂H | O | O | 1 | C | adapalene |
| B1-1003 | t-Bu | O | O | CO₂H | O | O | 1 | C | adapalene |
| B1-1004 | CF₃CH₂ | O | O | CO₂H | O | O | 1 | C | adapalene |
| B1-1005 | allyl | O | O | CO₂H | O | O | 1 | C | adapalene |
| B1-1006 | 4-(NO₂)Ph | O | O | CO₂H | O | O | 1 | C | adapalene |
| B1-1007 | PhS | O | O | CO₂H | O | O | 1 | C | adapalene |
| B1-1008 | N(Me)Ph | O | O | CO₂H | O | O | 1 | C | adapalene |
| B1-1009 | N(H)OMe | O | O | CO₂H | O | O | 1 | C | adapalene |
| B1-1010 | N(H)CO₂Et | O | O | CO₂H | O | O | 1 | C | adapalene |
| B1-1011 | Me | O | O | CO₂H | O | O | 1 | C | lansoprazole |
| B1-1012 | cyclohexyl | O | O | CO₂H | O | O | 1 | C | lansoprazole |
| B1-1013 | t-Bu | O | O | CO₂H | O | O | 1 | C | lansoprazole |
| B1-1014 | CF₃CH₂ | O | O | CO₂H | O | O | 1 | C | lansoprazole |
| B1-1015 | allyl | O | O | CO₂H | O | O | 1 | C | lansoprazole |
| B1-1016 | 4-(NO₂)Ph | O | O | CO₂H | O | O | 1 | C | lansoprazole |
| B1-1017 | PhS | O | O | CO₂H | O | O | 1 | C | lansoprazole |
| B1-1018 | N(Me)Ph | O | O | CO₂H | O | O | 1 | C | lansoprazole |
| B1-1019 | N(H)OMe | O | O | CO₂H | O | O | 1 | C | lansoprazole |
| B1-1020 | N(H)CO₂Et | O | O | CO₂H | O | O | 1 | C | lansoprazole |
| B1-1021 | Me | O | O | CO₂H | O | O | 1 | C | lovastatin |
| B1-1022 | cyclohexyl | O | O | CO₂H | O | O | 1 | C | lovastatin |
| B1-1023 | t-Bu | O | O | CO₂H | O | O | 1 | C | lovastatin |
| B1-1024 | CF₃CH₂ | O | O | CO₂H | O | O | 1 | C | lovastatin |
| B1-1025 | allyL | O | O | CO₂H | O | O | 1 | C | lovastatin |
| B1-1026 | 4-(NO₂)Ph | O | O | CO₂H | O | O | 1 | C | lovastatin |
| B1-1027 | PhS | O | O | CO₂H | O | O | 1 | C | lovastatin |
| B1-1028 | N(Me)Ph | O | O | CO₂H | O | O | 1 | C | lovastatin |
| B1-1029 | N(H)OMe | O | O | CO₂H | O | O | 1 | C | lovastatiin |
| B1-1030 | N(H)CO₂Et | O | O | CO₂H | O | O | 1 | C | lovastatin |
| H1-1031 | Me | O | O | CO₂H | O | O | 1 | C | warfarin |
| B1-1032 | cyclohexyl | O | O | CO₂H | O | O | 1 | C | warfarin |
| B1-1033 | t-Bu | O | O | CO₂H | O | O | 1 | C | warfarin |
| B1-1034 | CF₃CH₂ | O | O | CO₂H | O | O | 1 | C | warfarin |
| B1-1035 | allyl | O | O | CO₂H | O | O | 1 | C | warfarin |
| B1-1036 | 4-(NO₂)Ph | O | O | COAl | O | O | 1 | C | warfarin |
| B1-1037 | PhS | O | O | CO₂H | O | O | 1 | C | warfarin |
| B1-1038 | N(Me)Ph | O | O | CO₂H | O | O | 1 | C | warfarin |
| B1-1039 | N(H)OMe | O | O | CO₂H | O | O | 1 | C | warfarin |
| B1-1040 | N(H)CO₂Et | O | O | CO₂H | O | O | 1 | C | warfarin |
| B1-1041 | Me | O | O | CO₂H | — | — | 0 | N | tolteridine |
| B1-1042 | cycbohexyl | O | O | CO₂H | — | — | 0 | N | tolteridine |
| B1-1043 | t-Bu | O | O | CO₂H | — | — | 0 | N | tolteridine |
| B1-1044 | CF₃CH₂ | O | O | CO₂H | — | — | 0 | N | tolteridine |
| B1-1045 | allyl | O | O | CO₂H | — | — | 0 | N | tolteridine |
| B1-1046 | 4-(NO₂)Ph | O | O | CO₂H | — | — | 0 | N | tolteridine |
| B1-1047 | PhS | O | O | CO₂H | — | — | 0 | N | tolteridine |
| B1-1048 | N(Me)Ph | O | O | CO₂H | — | — | 0 | N | tolteridine |
| B1-1049 | N(H)OMe | O | O | CO₂H | — | — | 0 | N | tolteridine |
| B1-1050 | N(H)CO₂Et | O | O | CO₂H | — | — | 0 | N | tolteridine |
| B1-1051 | Me | O | O | CO₂H | — | — | 0 | N | ticlopidine |
| B1-1052 | cyclohexyl | O | O | CO₂H | — | — | 0 | N | ticlopidine |
| B1-1053 | t-Bu | O | O | CO₂H | — | — | 0 | N | ticlopidine |
| B1-1054 | CF₃CH₂ | O | O | CO₂H | — | — | 0 | N | ticlopidine |
| B1-1055 | allyl | O | O | CO₂H | — | — | 0 | N | ticiopidine |
| B1-1056 | 4-(NO₂)Ph | O | O | CO₂H | — | — | 0 | N | ticlopidine |
| B1-1057 | PhS | O | O | CO₂H | — | — | 0 | N | ticlopidine |
| B1-1058 | N(Me)Ph | O | O | CO₂H | — | — | 0 | N | ticlopidine |
| B1-1059 | N(H)OMe | O | O | CO₂H | — | — | 0 | N | ticlopidine |
| B1-1060 | N(H)CO₂Et | O | O | CO₂H | — | — | 0 | N | ticlopidine |
| B1-1061 | Me | O | O | CO₂H | — | — | 0 | N | sibutramine |
| B1-1062 | cyclohexyl | O | O | CO₂H | — | — | 0 | N | sibutramine |
| B1-1063 | t-Bu | O | O | CO₂H | — | — | 0 | N | sibutramine |
| B1-1064 | CF₃CH₂ | O | O | CO₂H | — | — | 0 | N | sibutramine |
| B1-1065 | allyl | O | O | CO₂H | — | — | 0 | N | sibutramine |
| B1-1066 | 4-(NO₂)Ph | O | O | CO₂H | — | — | 0 | N | sibutramine |
| B1-1067 | PhS | O | O | CO₂H | — | — | 0 | N | sibutramine |
| B1-1068 | N(Me)Ph | O | O | CO₂H | — | — | 0 | N | sibutramine |
| B1-1069 | N(H)OMe | O | O | CO₂H | — | — | 0 | N | sibutramine |
| B1-1070 | N(H)CO₂Et | O | O | CO₂H | — | — | 0 | N | sibutramine |
| B1-1071 | Me | O | O | CO₂H | — | — | 0 | N | propoxyphene |
| B1-1072 | cyclohexyl | O | O | CO₂H | — | — | 0 | N | propoxyphene |
| B1-1073 | t-Bu | O | O | CO₂H | — | — | 0 | N | propoxyphene |
| B1-1074 | CF₃CH₂ | O | O | CO₂H | — | — | 0 | N | propoxyphene |
| B1-1075 | allyl | O | O | CO₂H | — | — | 0 | N | propoxyphene |
| B1-1076 | 4-(NO₂)Ph | O | O | CO₂H | — | — | 0 | N | propoxyphene |
| B1-1077 | PhS | O | O | CO₂H | — | — | 0 | N | propoxyphene |
| B1-1078 | N(Me)Ph | O | O | CO₂H | — | — | 0 | N | propoxyphene |
| B1-1079 | N(H)OMe | O | O | CO₂H | — | — | 0 | N | propoxyphene |
| B1-1080 | N(H)CO₂Et | O | O | CO₂H | — | — | 0 | N | propoxyphene |
| B1-1081 | Me | O | O | CO₂H | — | — | 0 | N | nifurantin |
| B1-1082 | cyclohexyl | O | O | CO₂H | — | — | 0 | N | nifurantin |
| B1-1083 | t-Bu | O | O | CO₂H | — | — | 0 | N | nifurantin |
| B1-1084 | CF₃CH₂ | O | O | CO₂H | — | — | 0 | N | nifurantin |
| B1-1085 | allyl | O | O | CO₂H | — | — | 0 | N | nifurantin |
| B1-1086 | 4-(NO₂)Ph | O | O | CO₂H | — | — | 0 | N | nifurantin |
| B1-1087 | PhS | O | O | CO₂H | — | — | 0 | N | nifurantin |
| B1-1088 | N(Me)Ph | O | O | CO₂H | — | — | 0 | N | nifurantin |
| B1-1089 | N(H)OMe | O | O | CO₂H | — | — | 0 | N | nifurantin |
| B1-1090 | N(H)CO₂Et | O | O | CO₂H | — | — | 0 | N | nifurantin |
| B1-1091 | Me | O | O | CO₂H | — | — | 0 | N | nefazodone |
| B1-1092 | cyclohexyl | O | O | CO₂H | — | — | 0 | N | nefazodone |
| B1-1093 | t-Bu | O | O | CO₂H | — | — | 0 | N | nefazodone |
| B1-1094 | CF₃CH₂ | O | O | CO₂H | — | — | 0 | N | nefazodone |
| B1-1095 | allyl | O | O | CO₂H | — | — | 0 | N | nefazodone |
| B1-1096 | 4-(NO₂)Ph | O | O | CO₂H | — | — | 0 | N | nefazodone |
| B1-1097 | PhS | O | O | CO₂H | — | — | 0 | N | nefazodone |
| B1-1098 | N(Me)Ph | O | O | CO₂H | — | — | 0 | N | nefazodone |
| B1-1099 | N(H)OMe | O | O | CO₂H | — | — | 0 | N | nefazodone |
| B1-1100 | N(H)CO₂Et | O | O | CO₂H | — | — | 0 | N | nefazodone |
| B1-1101 | Me | O | O | CO₂H | — | — | 0 | N | donezapil |
| B1-1102 | cyclohexyl | O | O | CO₂H | — | — | 0 | N | donezapil |
| B1-1103 | t-Bu | O | O | CO₂H | — | — | 0 | N | donezapil |
| B1-1104 | CF₃CH₂ | O | O | CO₂H | — | — | 0 | N | donezapil |
| B1-1105 | allyl | O | O | CO₂H | — | — | 0 | N | donezapil |
| B1-1106 | 4-(NO₂)Ph | O | O | CO₂H | — | — | 0 | N | donezapil |
| B1-1107 | PhS | O | O | CO₂H | — | — | 0 | N | donezapil |
| B1-1108 | N(Me)PH | O | O | CO₂H | — | — | 0 | N | donezapil |
| B1-1109 | N(H)OMe | O | O | CO₂H | — | — | 0 | N | donezapil |
| B1-1110 | N(H)CO₂Et | O | O | CO₂H | — | — | 0 | N | donezapil |
| B1-1111 | Me | O | O | CO₂H | — | — | 0 | N | dicodid |
| B1-1112 | cyclohexyl | O | O | CO₂H | — | — | 0 | N | dicodid |
| B1-1113 | t-Bu | O | O | CO₂H | — | — | 0 | N | dicodid |
| B1-1114 | CF₃CH₂ | O | O | CO₂H | — | — | 0 | N | dicodid |
| B1-1115 | allyl | O | O | CO₂H | — | — | 0 | N | dicodid |
| B1-1116 | 4-(NO₂)Ph | O | O | CO₂H | — | — | 0 | N | dicodid |
| B1-1117 | PhS | O | O | CO₂H | — | — | 0 | N | dicodid |
| B1-1118 | N(Me)Ph | O | O | CO₂H | — | — | 0 | N | dicodid |
| B1-1119 | N(H)OMe | O | O | CO₂H | — | — | 0 | N | dicodid |
| B1-1120 | N(H)CO₂Et | O | O | CO₂H | — | — | 0 | N | dicodid |
| B1-1121 | Me | O | O | CO₂H | — | — | 0 | N | colchicine |
| B1-1122 | cyclohexyl | O | O | CO₂H | — | — | 0 | N | colchicine |
| B1-1123 | t-Bu | O | O | CO₂H | — | — | 0 | N | colchicine |
| B1-1124 | CF₃CH₂ | O | O | CO₂H | — | — | 0 | N | colchicine |
| B1-1125 | allyl | O | O | CO₂H | — | — | 0 | N | colchicine |
| B1-1126 | 4-(NO₂)Ph | O | O | CO₂H | — | — | 0 | N | colchicine |
| B1-1127 | PhS | O | O | CO₂H | — | — | 0 | N | colchicine |
| B1-1128 | N(Me)Ph | O | O | CO₂H | — | — | 0 | N | colchicine |
| B1-1129 | N(H)OMe | O | O | CO₂H | — | — | 0 | N | colchicine |
| B1-1130 | N(H)CO₂Et | O | O | CO₂H | — | — | 0 | N | colchicine |
| B1-1131 | Me | O | O | CO₂H | — | — | 0 | N | citalopram |
| B1-1132 | cyclohexyl | O | O | CO₂H | — | — | 0 | N | citalopram |
| B1-1133 | t-Bu | O | O | CO₂H | — | — | 0 | N | citalopram |
| B1-1134 | CF₃CH₂ | O | O | CO₂H | — | — | 0 | N | citalopram |
| B1-1135 | allyl | O | O | CO₂H | — | — | 0 | N | citalopram |
| B1-1136 | 4-(NO₂)Ph | O | O | CO₂H | — | — | 0 | N | citalopram |
| B1-1137 | PhS | O | O | CO₂H | — | — | 0 | N | citalopram |
| B1-1138 | N(Me)Ph | O | O | CO₂H | — | — | 0 | N | citalopram |
| B1-1139 | N(H)OMe | O | O | CO₂H | — | — | 0 | N | citalopram |
| B1-1140 | N(H)CO₂Et | O | O | CO₂H | — | — | 0 | N | citalopram |
| B1-1141 | Me | O | O | CO₂H | — | — | 0 | N | benzatropine |
| B1-1142 | cyclohexyl | O | O | CO₂H | — | — | 0 | N | benzatropine |
| B1-1143 | t-Bu | O | O | CO₂H | — | — | 0 | N | benzatropine |
| B1-1144 | CF₃CH₂ | O | O | CO₂H | — | — | 0 | N | benzatropine |
| B1-1145 | allyl | O | O | CO₂H | — | — | 0 | N | benzatropine |
| B1-1146 | 4-(NO₂)Ph | O | O | CO₂H | — | — | 0 | N | benzatropine |
| B1-1147 | PhS | O | O | CO₂H | — | — | 0 | N | benzatropine |
| B1-1148 | N(Me)Ph | O | O | CO₂H | — | — | 0 | N | benzatropine |
| B1-1149 | N(H)OMe | O | O | CO₂H | — | — | 0 | N | benzatropine |
| B1-1150 | N(H)CO₂Et | O | O | CO₂H | — | — | 0 | N | benzatropine |
| B1-1151 | EtS | O | O | CO₂H | — | — | 0 | N | bisacodyl |
| B1-1152 | EtO | O | O | CO₂H | — | — | 0 | N | bisacodyl |
| B1-1153 | t-BuO | O | O | CO₂H | — | — | 0 | N | bisacodyl |
| B1-1154 | cyclohexylO | O | O | CO₂H | — | — | 0 | N | bisacodyl |
| B1-1155 | iPrO | O | O | CO₂H | — | — | 0 | N | bisacodyl |
| B1-1156 | PhO | O | O | CO₂H | — | — | 0 | N | bisacodyl |
| B1-1157 | (HOCH₂— | O | O | CO₂H | — | — | 0 | N | bisacodyl |
| | CH₂)₂N | | | | | | | | |
| B1-1158 | N(H)NMe₂ | O | O | CO₂H | — | — | 0 | N | bisacodyl |
| B1-1159 | 4-(OH)Ph | O | O | CO₂H | — | — | 0 | N | bisacodyl |
| B1-1160 | 4-(NH₂)Ph | O | O | CO₂H | — | — | 0 | N | bisacodyl |
| B1-1161 | EtS | O | O | CO₂H | — | — | 0 | N | bupivacaine |
| B1-1162 | EtO | O | O | CO₂H | — | — | 0 | N | bupivacaine |
| B1-1163 | t-BuO | O | O | CO₂H | — | — | 0 | N | bupivacaine |
| B1-1164 | cyclohexylO | O | O | CO₂H | — | — | 0 | N | bupivacaine |
| B1-1165 | iPrO | O | O | CO₂H | — | — | 0 | N | bupivacaine |
| B1-1166 | PhO | O | O | CO₂H | — | — | 0 | N | bupivacaine |
| B1-1167 | (HOCH₂— | O | O | CO₂H | — | — | 0 | N | bupivacaine |
| | CH₂)₂N | | | | | | | | |
| B1-1168 | N(H)NMe₂ | O | O | CO₂H | — | — | 0 | N | bupivacaine |
| B1-1169 | 4-(OH)Ph | O | O | CO₂H | — | — | 0 | N | bupivacaine |
| B1-1170 | 4-(NH₂)Ph | O | O | CO₂H | — | — | 0 | N | bupivacaine |
| B1-1171 | EtS | O | O | CO₂H | — | — | 0 | N | chloroprocaine |
| B1-1172 | EtO | O | O | CO₂H | — | — | 0 | N | chloroprocaine |
| B1-1173 | t-BuO | O | O | CO₂H | — | — | 0 | N | chloroprocaine |
| B1-1174 | cyclohexylO | O | O | CO₂H | — | — | 0 | N | chloroprocaine |
| B1-1175 | iPrO | O | O | CO₂H | — | — | 0 | N | chloroprocaine |
| B1-1176 | PhO | O | O | CO₂H | — | — | 0 | N | chloroprocaine |
| B1-1177 | (HOCH₂— | O | O | CO₂H | — | — | 0 | N | chloroprocaine |
| | CH₂)₂N | | | | | | | | |
| B1-1178 | N(H)NMe₂ | O | O | CO₂H | — | — | 0 | N | chloroprocaine |
| B1-1179 | 4-(OH)Ph | O | O | CO₂H | — | — | 0 | N | chloroprocaine |
| B1-1180 | 4-(NH₂)Ph | O | O | CO₂H | — | — | 0 | N | chloroprocaine |
| B1-1181 | EtS | O | O | CO₂H | — | — | 0 | N | tetracaine |
| B1-1182 | EtO | O | O | CO₂H | — | — | 0 | N | tetracaine |
| B1-1183 | t-BuO | O | O | CO₂H | — | — | 0 | N | tetracaine |
| B1-1184 | cyclohexylO | O | O | CO₂H | — | — | 0 | N | tetracaine |
| B1-1185 | iPrO | O | O | CO₂H | — | — | 0 | N | tetracaine |
| B1-1186 | PhO | O | O | CO₂H | — | — | 0 | N | tetracaine |
| B1-1187 | (HOCH₂— | O | O | CO₂H | — | — | 0 | N | tetracaine |
| | CH₂)₂N | | | | | | | | |
| B1-1188 | N(H)NMe₂ | O | O | CO₂H | — | — | 0 | N | tetracaine |
| B1-1189 | 4-(OH)Ph | O | O | CO₂H | — | — | 0 | N | tetracaine |
| B1-1190 | 4-(NH₂)Ph | O | O | CO₂H | — | — | 0 | N | tetracaine |
| B1-1191 | EtS | O | O | CO₂H | — | — | 0 | N | acrivistine |
| B1-1192 | EtO | O | O | CO₂H | — | — | 0 | N | acrivistine |
| B1-1193 | t-BuO | O | O | CO₂H | — | — | 0 | N | acrivistine |
| B1-1194 | cyclohexylO | O | O | CO₂H | — | — | 0 | N | acrivistine |
| B1-1195 | iPrO | O | O | CO₂H | — | — | 0 | N | acrivistine |
| B1-1196 | PhO | O | O | CO₂H | — | — | 0 | N | acrivistine |
| B1-1197 | (HOCH₂— | O | O | CO₂H | — | — | 0 | N | acrivistine |
| | CH₂)₂N | | | | | | | | |
| B1-1198 | N(H)NMe₂ | O | O | CO₂H | — | — | 0 | N | acrivistine |
| B1-1199 | 4-(OH)Ph | O | O | CO₂H | — | — | 0 | N | acrivistine |
| B1-1200 | 4-(NH₂)Ph | O | O | CO₂H | — | — | 0 | N | acrivistine |
| B1-1201 | EtS | O | O | CO₂H | — | — | 0 | N | amiodarone |
| B1-1202 | EtO | O | O | CO₂H | — | — | 0 | N | amiodarone |
| B1-1203 | t-BuO | O | O | CO₂H | — | — | 0 | N | amiodarone |
| B1-1204 | cyclohexylO | O | O | CO₂H | — | — | 0 | N | amiodarone |
| B1-1205 | iPrO | O | O | CO₂H | — | — | 0 | N | amiodarone |
| B1-1206 | PhO | O | O | CO₂H | — | — | 0 | N | amiodarone |
| B1-1207 | (HOCH₂— | O | O | CO₂H | — | — | 0 | N | amiodarone |
| | CH₂)₂N | | | | | | | | |
| B1-1208 | N(H)NMe₂ | O | O | CO₂H | — | — | 0 | N | amiodarone |
| B1-1209 | 4-(OH)Ph | O | O | CO₂H | — | — | 0 | N | amiodarone |
| B1-1210 | 4-(NH₂)Ph | O | O | CO₂H | — | — | 0 | N | amiodarone |
| B1-1211 | EtS | O | O | CO₂H | — | — | 0 | N | amitriptyline |
| B1-1212 | EtO | O | O | CO₂H | — | — | 0 | N | amitriptyline |
| B1-1213 | t-BuO | O | O | CO₂H | — | — | 0 | N | amitriptyline |
| B1-1214 | cyclohexylO | O | O | CO₂H | — | — | 0 | N | amitriptyline |
| B1-1215 | iPrO | O | O | CO₂H | — | — | 0 | N | amitriptyline |
| B1-1216 | PhO | O | O | CO₂H | — | — | 0 | N | amitriptyline |
| B1-1217 | (HOCH₂— | O | O | CO₂H | — | — | 0 | N | amitriptyline |
| | CH₂)₂N | | | | | | | | |
| B1-1218 | N(H)NMe₂ | O | O | CO₂H | — | — | 0 | N | amitriptyhine |
| B1-1219 | 4-(OH)Ph | O | O | CO₂H | — | — | 0 | N | amitriptyline |
| B1-1220 | 4-(NH₂)Ph | O | O | CO₂H | — | — | 0 | N | amitriptyline |
| B1-1221 | EtS | O | O | CO₂H | — | — | 0 | N | amrinone |
| B1-1222 | EtO | O | O | CO₂H | — | — | 0 | N | amrinone |
| B1-1223 | t-BuO | O | O | CO₂H | — | — | 0 | N | amrinone |
| B1-1224 | cyclohexylO | O | O | CO₂H | — | — | 0 | N | amrinone |
| B1-1225 | iPrO | O | O | CO₂H | — | — | 0 | N | amrinone |
| B1-1226 | PhO | O | O | CO₂H | — | — | 0 | N | amrinone |
| B1-1227 | (HOCH₂— | O | O | CO₂H | — | — | 0 | N | amrinone |
| | CH₂)₂N | | | | | | | | |
| B1-1228 | N(H)NMe₂ | O | O | CO₂H | — | — | 0 | N | amrinone |
| B1-1229 | 4-(OH)Ph | O | O | CO₂H | — | — | 0 | N | amrinone |
| B1-1230 | 4-(NH₂)Ph | O | O | CO₂H | — | — | 0 | N | amrinone |
| B1-1231 | EtS | O | O | CO₂H | — | — | 0 | N | atropine |
| B1-1232 | EtO | O | O | CO₂H | — | — | 0 | N | atropine |
| B1-1233 | t-BuO | O | O | CO₂H | — | — | 0 | N | atropine |
| B1-1234 | cyclohexylO | O | O | CO₂H | — | — | 0 | N | atropine |
| B1-1235 | iPrO | O | O | CO₂H | — | — | 0 | N | atropine |
| B1-1236 | PhO | O | O | CO₂H | — | — | 0 | N | atropine |
| B1-1237 | (HOCH₂— | O | O | CO₂H | — | — | 0 | N | atropine |
| | CH₂)₂N | | | | | | | | |
| B1-1238 | N(H)NMe₂ | O | O | CO₂H | — | — | 0 | N | atropine |
| B1-1239 | 4-(OH)Ph | O | O | CO₂H | — | — | 0 | N | atropine |
| B1-1240 | 4-(NH₂)Ph | O | O | CO₂H | — | — | 0 | N | atropine |
| B1-1241 | EtS | O | O | CO₂H | — | — | 0 | N | benzphetamine |
| B1-1242 | EtO | O | O | CO₂H | — | — | 0 | N | benzphetamine |
| B1-1243 | t-BuO | O | O | CO₂H | — | — | 0 | N | benzphetamine |
| B1-1244 | cyclohexylO | O | O | CO₂H | — | — | 0 | N | benzphetamine |
| B1-1245 | iPrO | O | O | CO₂H | — | — | 0 | N | benzphetamine |
| B1-1246 | PhO | O | O | CO₂H | — | — | 0 | N | benzphetamine |
| B1-1247 | (HOCH₂— | O | O | CO₂H | — | — | 0 | N | benzphetamine |
| | CH₂)₂N | | | | | | | | |
| B1-1248 | N(H)NMe₂ | O | O | CO₂H | — | — | 0 | N | benzphetamine |
| B1-1249 | 4-(OH)Ph | O | O | CO₂H | — | — | 0 | N | benzphetamine |
| B1-1250 | 4-(NH₂)Ph | O | O | CO₂H | — | — | 0 | N | benzphetamine |
| B1-1251 | EtS | O | O | CO₂H | — | — | 0 | N | beperiden |
| B1-1252 | EtO | O | O | CO₂H | — | — | 0 | N | beperiden |
| B1-1253 | t-Bu0 | O | O | CO₂H | — | — | 0 | N | beperiden |
| B1-1254 | cyclohexylO | O | O | CO₂H | — | — | 0 | N | beperiden |
| B1-1255 | iPrO | O | O | CO₂H | — | — | 0 | N | beperiden |
| B1-1256 | PhO | O | O | CO₂H | — | — | 0 | N | beperiden |
| B1-1257 | (HOCH₂— | O | O | CO₂H | — | — | 0 | N | beperiden |
| | CH₂)₂N | | | | | | | | |
| B1-1258 | N(H)NMe₂ | O | O | CO₂H | — | — | 0 | N | beperiden |
| B1-1259 | 4-(OH)Ph | O | O | CO₂H | — | — | 0 | N | beperiden |
| B1-1260 | 4-(NH₂)Ph | O | O | CO₂H | — | — | 0 | N | beperiden |
| B1-1261 | EtS | O | O | CO₂H | — | — | 0 | N | bromopheniramine |
| B1-1262 | EtO | O | O | CO₂H | — | — | 0 | N | bromopheniramine |
| B1-1263 | t-BuO | O | O | CO₂H | — | — | 0 | N | bromopheniramine |
| B1-1264 | cyclohexylO | O | O | CO₂H | — | — | 0 | N | bromopheniramine |
| B1-1265 | iPrO | O | O | CO₂H | — | — | 0 | N | bromopheniramine |
| B1-1266 | PhO | O | O | CO₂H | — | — | 0 | N | bromopheniramine |
| B1-1267 | (HOCH₂— | O | O | CO₂H | — | — | 0 | N | bromopheniramine |
| | CH₂)₂N | | | | | | | | |
| B1-1268 | N(H)NMe₂ | O | O | CO₂H | — | — | 0 | N | bromopheniramine |
| B1-1269 | 4-(OH)Ph | O | O | CO₂H | — | — | 0 | N | bromopheniramine |
| B1-1270 | 4-(NH₂)Ph | O | O | CO₂H | — | — | 0 | N | bromopheniramine |
| B1-1271 | EtS | O | O | CO₂H | — | — | 0 | N | clemastine |
| B1-1272 | EtO | O | O | CO₂H | — | — | 0 | N | clemastine |
| B1-1273 | t-BuO | O | O | CO₂H | — | — | 0 | N | clemastine |
| B1-l274 | cyclohexylO | O | 0 | CO₂H | — | — | 0 | N | clemastine |
| B1-1275 | iPrO | O | O | CO₂H | — | — | 0 | N | clemastine |
| B1-1276 | PhO | O | O | C021i | — | — | 0 | N | clemastine |
| B1-1277 | (HOCH₂— | O | O | CO₂H | — | — | 0 | N | clemastine |
| | CH₂)₂N | | | | | | | | |
| B1-1278 | N(H)NMe₂ | O | O | CO₂H | — | — | 0 | N | clemastine |
| B1-1279 | 4-(OH)Ph | O | O | CO₂H | — | — | 0 | N | clemastine |
| B1-1280 | 4-(NH₂)Ph | O | O | CO₂H | — | — | 0 | N | clemastine |
| B1-1281 | EtS | O | O | CO₂H | — | — | 0 | N | clomiphene |
| B1-1282 | EtO | O | O | CO₂H | — | — | 0 | N | clomiphene |
| B1-1283 | t-BuO | O | O | CO₂H | — | — | 0 | N | clomiphene |
| B1-1284 | cyclohexylO | O | O | CO₂H | — | — | 0 | N | clomiphene |
| B1-1285 | iPrO | O | O | CO₂H | — | — | 0 | N | clomiphene |
| B1-1286 | PhO | O | O | CO₂H | — | — | 0 | N | clomiphene |
| B1-1287 | (HOCH₂— | O | O | CO₂H | — | — | 0 | N | clomiphene |
| | CH₂)₂N | | | | | | | | |
| B1-1288 | N(H)NMe₂ | O | O | CO₂H | — | — | 0 | N | clomiphene |
| B1-1289 | 4-(OH)Ph | O | O | CO₂H | — | — | 0 | N | clomiphene |
| B1-1290 | 4-(NH₂)Ph | O | O | CO₂H | — | — | 0 | N | clomiphene |
| B1-1291 | EtS | O | O | CO₂H | — | — | 0 | N | cyclobenzaprine |
| B1-1292 | EtO | O | O | CO₂H | — | — | 0 | N | cyclobenzaprine |
| B1-1293 | t-BuO | O | O | CO₂H | — | — | 0 | N | cyclobenzaprine |
| B1-1294 | cyclohexylO | O | O | CO₂H | — | — | 0 | N | cyclobenzaprine |
| B1-1295 | iPrO | O | O | CO₂H | — | — | 0 | N | cyclobenzaprine |
| B1-1296 | PhO | O | O | CO₂H | — | — | 0 | N | cyclobenzaprine |
| B1-1297 | (HOCH₂— | O | O | CO₂H | — | — | 0 | N | cyclobenzaprine |
| | CH₂)₂N | | | | | | | | |
| B1-1298 | N(H)NMe₂ | O | O | CO₂H | — | — | 0 | N | cyclobenzaprine |
| B1-1299 | 4-(OH)Ph | O | O | CO₂H | — | — | 0 | N | cyclobenzaprine |
| B1-1300 | 4-(NH₂)Ph | O | O | CO₂H | — | — | 0 | N | cyclobenzaprine |
| B1-1301 | EtS | O | O | CO₂H | — | — | 0 | N | cyclopentolate |
| B1-1302 | EtO | O | O | CO₂H | — | — | 0 | N | cyclopentolate |
| B1-1303 | t-BuO | O | O | CO₂H | — | — | 0 | N | cyclopentolate |
| B1-1304 | cyclohexylO | O | O | CO₂H | — | — | 0 | N | cyclopentolate |
| B1-1305 | iPrO | O | O | CO₂H | — | — | 0 | N | cyclopentolate |
| B1-1306 | PhO | O | O | CO₂H | — | — | 0 | N | cyclopentolate |
| B1-1307 | (HOCH₂— | O | O | CO₂H | — | — | 0 | N | cyclopentolate |
| | CH₂)₂N | | | | | | | | |
| B1-1308 | N(H)NMe₂ | O | O | CO₂H | — | — | 0 | N | cyclopentolate |
| B1-1309 | 4-(OH)Ph | O | O | CO₂H | — | — | 0 | N | cyclopentolate |
| B1-1310 | 4-(NH₂)Ph | O | O | CO₂H | — | — | 0 | N | cyclopentolate |
| B1-1311 | EtS | O | O | CO₂H | — | — | 0 | N | dicyclomine |
| B1-1312 | EtO | O | O | CO₂H1 | — | — | 0 | N | dicyclomine |
| B1-1313 | t-Bu0 | O | O | CO₂H | — | — | 0 | N | dicyclomine |
| B1-1314 | cyclohexylO | O | O | CO₂H | — | — | 0 | N | dicyclomine |
| B1-1315 | iPrO | O | O | CO₂H | — | — | 0 | N | dicyclomine |
| B1-1316 | PhO | O | O | CO₂H | — | — | 0 | N | dicyclomine |
| B1-1317 | (HOCH₂— | O | O | CO₂H | — | — | 0 | N | dicyclomine |
| | CH₂)₂N | | | | | | | | |
| B1-1318 | N(H)NMe₂ | O | O | CO₂H | — | — | 0 | N | dicyclomine |
| B1-1319 | 4-(OH)Ph | O | O | CO₂H | — | — | 0 | N | dicyclomine |
| B1-1320 | 4-(NH₂)Ph | O | O | CO₂H | — | — | 0 | N | dicyclomine |
| B1-1321 | EtS | O | O | CO₂H | — | — | 0 | N | diethylproprion |
| B1-1322 | EtO | O | O | CO₂H | — | — | 0 | N | diethylproprion |
| B1-1323 | t-BuO | O | O | CO₂H | — | — | 0 | N | diethylproprion |
| B1-1324 | cyclohexylO | O | O | CO₂H | — | — | 0 | N | diethylproprion |
| B1-1325 | iPrO | O | O | CO₂H | — | — | 0 | N | diethylproprion |
| B1-1326 | Ph0 | O | O | CO₂H | — | — | 0 | N | diethylproprion |
| B1-1327 | (HOCH₂— | O | O | CO₂H | — | — | 0 | N | diethylproprion |
| | CH₂)₂N | | | | | | | | |
| B1-1328 | N(H)NMe₂ | O | O | CO₂H | — | — | 0 | N | diethylproprion |
| B1-1329 | 4-(OH)Ph | O | O | CO₂H | — | — | 0 | N | diethylproprion |
| B1-1330 | 4-(NH₂)Ph | O | O | CO₂H | — | — | 0 | N | diethylproprion |
| B1-1331 | EtS | O | O | CO₂H | — | — | 0 | N | diltiazem |
| B1-1332 | EtO | O | O | CO₂H | — | — | 0 | N | diltiazem |
| B1-1333 | t-BuO | O | O | CO₂H | — | — | 0 | N | diltiazem |
| B1-1334 | cyclohexylO | O | O | CO₂H | — | — | 0 | N | diltiazem |
| B1-1335 | iPrO | O | O | CO₂H | — | — | 0 | N | diltiazem |
| B1-1336 | PhO | O | O | CO₂H | — | — | 0 | N | diltiazem |
| B1-1337 | (HOCH₂— | O | O | CO₂H | — | — | 0 | N | diltiazem |
| | CH₂)₂N | | | | | | | | |
| B1-1338 | N(H)NMe₂ | O | O | CO₂H | — | — | 0 | N | diltiazem |
| B1-1339 | 4-(OH)Ph | O | O | CO₂H | — | — | 0 | N | diltiazem |
| B1-1340 | 4-(NH₂)Ph | O | O | CO₂H | — | — | 0 | N | diltiazem |
| B1-1341 | EtS | O | O | CO₂H | — | — | 0 | N | diphenhydramine |
| B1-1342 | EtO | O | O | CO₂H | — | — | 0 | N | diphenhydramine |
| B1-1343 | t-BuO | O | O | CO₂H | — | — | 0 | N | diphenhydramine |
| B1-1344 | cyclohexylO | O | O | CO₂H | — | — | 0 | N | diphenhydramine |
| B1-1345 | iPrO | O | O | CO₂H | — | — | 0 | N | diphenhydramine |
| B1-1346 | PhO | O | O | CO₂H | — | — | 0 | N | diphenhydramine |
| B1-1347 | (HOCH₂— | O | O | CO₂H | — | — | 0 | N | diphenhydramine |
| | CH₂)₂N | | | | | | | | |
| B1-1348 | N(H)NMe₂ | O | O | CO₂H | — | — | 0 | N | diphenhydramine |
| B1-1349 | 4-(OH)Ph | O | O | CO₂H | — | — | 0 | N | diphenhydramine |
| B1-1350 | 4-(NH₂)Ph | O | O | CO₂H | — | — | 0 | N | diphenhydramine |
| B1-1351 | EtS | O | O | CO₂H | — | — | 0 | N | diphenidol |
| B1-1352 | EtO | O | O | CO₂H | — | — | 0 | N | diphenidol |
| B1-1353 | t-BuO | O | O | CO₂H | — | — | 0 | N | diphenidol |
| B1-1354 | cyclohexylO | O | O | GO2H | — | — | 0 | N | diphenidol |
| B1-1355 | iPr0 | O | O | CO₂H | — | — | 0 | N | diphenidol |
| B1-1356 | PhO | O | O | CO₂H | — | — | 0 | N | diphenidol |
| B1-1357 | (HOCH₂— | O | O | CO₂H | — | — | 0 | N | diphenidol |
| | CH₂)₂N | | | | | | | | |
| B1-1358 | N(H)NMe₂ | O | O | CO₂H | — | — | 0 | N | diphenidol |
| B1-1359 | 4-(OH)Ph | O | O | CO₂H | — | — | 0 | N | diphenidol |
| B1-1360 | 4-(NH₂)Ph | O | O | CO₂H | — | — | 0 | N | diphenidol |
| B1-1361 | EtS | O | O | CO₂H | — | — | 0 | N | diphenoxylate |
| B1-1362 | Et0 | O | O | CO₂H | — | — | 0 | N | diphenoxylate |
| B1-1363 | t-BuO | O | O | CO₂H | — | — | 0 | N | diphenoxylate |
| B1-1364 | cyclohexylO | O | O | CO₂H | — | — | 0 | N | diphenoxylate |
| B1-1365 | iPrO | O | O | CO₂H | — | — | 0 | N | diphenoxylate |
| B1-1366 | PhO | O | O | CO₂H | — | — | 0 | N | diphenoxylate |
| B1-1367 | (HOCH₂— | O | O | CO₂H | — | — | 0 | N | diphenoxylate |
| | CH₂)₂N | | | | | | | | |
| B1-1368 | N(H)NMe₂ | O | O | CO₂H | — | — | 0 | N | diphenoxylate |
| B1-1369 | 4-(OH)Ph | O | O | CO₂H | — | — | 0 | N | diphenoxylate |
| B1-1370 | 4-(NH₂)Ph | O | O | CO₂H | — | — | 0 | N | diphenoxylate |
| B1-1371 | EtS | O | O | CO₂H | — | — | 0 | N | doxapram |
| B1-1372 | EtO | O | O | CO₂H | — | — | 0 | N | doxapram |
| B1-1373 | t-BuO | O | O | CO₂H | — | — | 0 | N | doxapram |
| B1-1374 | cyclohexylO | O | O | CO₂H | — | — | 0 | N | doxapram |
| B1-1375 | iPrO | O | O | CO₂H | — | — | 0 | N | doxapram |
| B1-1376 | PhO | O | O | CO₂H | — | — | 0 | N | doxapram |
| B1-1377 | (HOCH₂— | O | O | CO₂H | — | — | 0 | N | doxapram |
| | CH₂)₂N | | | | | | | | |
| B1-1378 | N(H)NMe2 | O | O | CO₂H | — | — | 0 | N | doxapram |
| B1-1379 | 4-(OH)Ph | O | O | CO₂H | — | — | 0 | N | doxapram |
| B1-1380 | 4-(NH₂)Ph | O | O | CO₂H | — | — | 0 | N | doxapram |
| B1-1381 | EtS | O | O | CO₂H | — | — | 0 | N | doxepin |
| B1-1382 | EtO | O | O | CO₂H | — | — | 0 | N | doxepin |
| B1-1383 | t-BuO | O | O | CO₂H | — | — | 0 | N | doxepin |
| B1-1384 | cyclohexylO | O | O | CO₂H | — | — | 0 | N | doxepin |
| B1-1385 | iPrO | O | O | CO₂H | — | — | 0 | N | doxepm |
| B1-1386 | PhO | O | O | CO₂H | — | — | 0 | N | doxepin |
| B1-1387 | (HOCH₂— | O | O | CO₂H | — | — | 0 | N | doxepin |
| | CH₂)₂N | | | | | | | | |
| B1-1388 | N(H)NMe₂ | O | O | CO₂H | — | — | 0 | N | doxepin |
| B1-1389 | 4-(OH)Ph | O | O | CO₂H | — | — | 0 | N | doxepin |
| B1-1390 | 4-(NH₂)Ph | O | O | CO₂H | — | — | 0 | N | doxepin |
| B1-1391 | EtS | O | O | CO₂H | — | — | 0 | N | fentanyl |
| B1-1392 | EtO | O | O | CO₂H | — | — | 0 | N | fentanyl |
| B1-1393 | t-BuO | O | O | CO₂H | — | — | 0 | N | fentanyl |
| B1-1394 | cyclohexylO | O | O | CO₂H | — | — | 0 | N | fentanyl |
| B1-1395 | iPrO | O | O | CO₂H | — | — | 0 | N | fentanyl |
| B1-1396 | PhO | O | O | CO₂H | — | — | 0 | N | fentanyl |
| B1-1397 | (HOCH₂— | O | O | CO₂H | — | — | 0 | N | fentanyl |
| | CH₂)₂N | | | | | | | | |
| B1-1398 | N(H)NMe₂ | O | O | CO₂H | — | — | 0 | N | fentanyl |
| B1-1399 | 4-(OH)Ph | O | O | CO₂H | — | — | 0 | N | fentanyl |
| B1-1400 | 4-(NH₂)Ph | O | O | CO₂H | — | — | 0 | N | fentanyl |
| B1-1401 | EtS | O | O | CO₂H | — | — | 0 | N | flavoxate |
| B1-1402 | Et0 | O | O | CO₂H | — | — | 0 | N | flavoxate |
| B1-1403 | t-BuO | O | O | CO₂H | — | — | 0 | N | flavoxate |
| B1-1404 | cyclohexylO | O | O | CO₂H | — | — | 0 | N | flavoxate |
| B1-1405 | iPrO | O | O | CO₂H | — | — | 0 | N | flavoxate |
| B1-1406 | PhO | O | O | CO₂H | — | — | 0 | N | flavoxate |
| B1-1407 | (HOCH₂— | O | O | CO₂H | — | — | 0 | N | flavoxate |
| | CH₂)₂N | | | | | | | | |
| B1-1408 | N(H)NMe₂ | O | O | CO₂H | — | — | 0 | N | flavoxate |
| B1-1409 | 4-(OH)PhO | O | O | CO₂H | — | — | 0 | N | flavoxate |
| B1-1410 | 4-(NH₂)Ph | O | O | CO₂H | — | — | 0 | N | flavoxate |
| B1-1411 | EtS | O | O | CO₂H | — | — | 0 | N | flurazepam |
| B1-1412 | EtO | O | O | CO₂H | — | — | 0 | N | flurazepam |
| B1-1413 | t-BuO | O | O | CO₂H | — | — | 0 | N | flurazepam |
| B1-1414 | cyclohexylO | O | O | CO₂H | — | — | 0 | N | flurazepam |
| B1-1415 | iPrO | O | O | CO₂H | — | — | 0 | N | flurazepam |
| B1-1416 | PhO | O | O | CO₂H | — | — | 0 | N | flurazepam |
| B1-1417 | (HOCH₂— | O | O | CO₂H | — | — | 0 | N | flurazepam |
| | CH₂)₂N | | | | | | | | |
| B1-1418 | N(H)NMe₂ | O | O | CO₂H | — | — | 0 | N | flurazepam |
| B1-1419 | 4-(OH)Ph | O | O | CO₂H | — | — | 0 | N | flurazepam |
| B1-1420 | 4-(NH₂)Ph | O | O | CO₂H | — | — | 0 | N | flurazepam |
| B1-1421 | EtS | O | O | CO₂H | — | — | 0 | N | levomethadyl |
| B1-1422 | EtO | O | O | CO₂H | — | — | 0 | N | levomethadyl |
| B1-1423 | t-BuO | O | O | CO₂H | — | — | 0 | N | levomethadyl |
| B1-1424 | cyclohexylO | O | O | CO₂H | — | — | 0 | N | levornethadyl |
| B1-1425 | iPrO | O | O | CO₂H | — | — | 0 | N | levomethadyl |
| B1-1426 | PhO | O | O | CO₂H | — | — | 0 | N | levomethadyl |
| B1-1427 | (HOCH₂— | O | O | CO₂H | — | — | 0 | N | levomethadyl |
| | CH₂)₂N | | | | | | | | |
| B1-1428 | N(H)NMe₂ | O | O | CO₂H | — | — | 0 | N | levomethadyl |
| B1-1429 | 4-(OH)Ph | O | O | CO₂H | — | — | 0 | N | levomethadyl |
| B1-1430 | 4-(NH₂)Ph | O | O | CO₂H | — | — | 0 | N | levomethadyl |
| B1-1431 | EtS | O | O | CO₂H | — | — | 0 | N | loratadine |
| B1-1432 | EtO | O | O | CO₂H | — | — | 0 | N | loratadine |
| B1-1433 | t-BuO | O | O | CO₂H | — | — | 0 | N | loratadine |
| B1-1434 | cyclohexylO | O | O | CO₂H | — | — | 0 | N | loratadine |
| B1-1435 | iPrO | O | O | CO₂H | — | — | 0 | N | loratadine |
| B1-1436 | PhO | O | O | CO₂H | — | — | 0 | N | loratadine |
| B1-1437 | (HOCH₂— | O | O | CO₂H | — | — | 0 | N | loratadine |
| | CH₂)₂N | | | | | | | | |
| B1-1438 | N(H)NMe₂ | O | O | CO₂H | — | — | 0 | N | loratadine |
| B1-1439 | 4-(OH)Ph | O | O | CO₂H | — | — | 0 | N | loratadine |
| B1-1440 | 4-(NH₂)Ph | O | O | CO₂H | — | — | 0 | N | loratadine |
| B1-1441 | EtS | O | O | CO₂H | — | — | 0 | N | mechlorethamine |
| B1-1442 | EtO | O | O | CO₂H | — | — | 0 | N | mechlorethamine |
| B1-1443 | t-BuO | O | O | CO₂H | — | — | 0 | N | mechlorethamine |
| B1-1444 | cyclohexylO | O | O | CO₂H | — | — | 0 | N | mechlorethamine |
| B1-1445 | iPrO | O | O | CO₂H | — | — | 0 | N | mechlorethamirie |
| B1-1446 | PhO | O | O | CO₂H | — | — | 0 | N | mechlorethamine |
| B1-1447 | (HOCH₂— | O | O | CO₂H | — | — | 0 | N | mechlorethamine |
| | CH₂)₂N | | | | | | | | |
| B1-1448 | N(H)NMe₂ | O | O | CO₂H | — | — | 0 | N | mechlorethamine |
| B1-1449 | 4-(OH)Ph | O | O | CO₂H | — | — | 0 | N | mechlorethainine |
| B1-1450 | 4-(NH₂)Ph | O | O | CO₂H | — | — | 0 | N | mechlorethamine |
| B1-1451 | EtS | O | O | CO₂H | — | — | 0 | N | meperidine |
| B1-1452 | EtO | O | O | CO₂H | — | — | 0 | N | meperidine |
| B1-1453 | t-BuO | O | O | CO₂H | — | — | 0 | N | meperidine |
| B1-1454 | cyclohexylO | O | O | CO₂H | — | — | 0 | N | meperidine |
| B1-1455 | iPrO | O | O | CO₂H | — | — | 0 | N | meperidine |
| B1-1456 | PhO | O | O | CO₂H | — | — | 0 | N | meperidine |
| B1-1457 | (HOCH₂— | O | O | CO₂H | — | — | 0 | N | meperidine |
| | CH₂)₂N | | | | | | | | |
| B1-1458 | N(H)NMe₂ | O | O | CO₂H | — | — | 0 | N | meperidine |
| B1-1459 | 4-(OH)Ph | O | O | CO₂H | — | — | 0 | N | meperidine |
| B1-1460 | 4-(NH₂)Ph | O | O | CO₂H | — | — | 0 | N | meperidine |
| B1-1461 | EtS | O | O | CO₂H | — | — | 0 | N | mepivacaine |
| B1-1462 | EtO | O | O | CO₂H | — | — | 0 | N | mepivacaine |
| B1-1463 | t-BuO | O | O | CO₂H | — | — | 0 | N | mepivacaine |
| B1-1464 | cyclohexylO | O | O | CO₂H | — | — | 0 | N | mepivacaine |
| B1-1465 | iPrO | O | O | CO₂H | — | — | 0 | N | mepivacame |
| B1-1466 | PhO | O | O | CO₂H | — | — | 0 | N | mepivacaine |
| B1-1467 | (HOCH₂— | O | O | CO₂H | — | — | 0 | N | mepivacaine |
| | CH₂)₂N | | | | | | | | |
| B1-1468 | N(H)NMe₂ | O | O | CO₂H | — | — | 0 | N | mepivacaine |
| B1-1469 | 4-(OH)Ph | O | O | CO₂H | — | — | 0 | N | mepivacaine |
| B1-1470 | 4-(NH₂)Ph | O | O | CO₂H | — | — | 0 | N | mepivacaine |
| B1-1471 | EtS | O | O | CO₂H | — | — | 0 | N | methadone |
| B1-1472 | EtO | O | O | CO₂H | — | — | 0 | N | methadone |
| B1-1473 | t-BuO | O | O | CO₂H | — | — | 0 | N | methadone |
| B1-1474 | cyclohexylO | O | O | CO₂H | — | — | 0 | N | methadone |
| B1-1475 | iPrO | O | O | CO₂H | — | — | 0 | N | methadone |
| B1- 1476 | PhO | O | O | CO₂H | — | — | 0 | N | methadone |
| B1-1477 | (HOCH₂— | O | O | COdI | — | — | 0 | N | methadone |
| | CH₂)₂N | | | | | | | | |
| B1-1478 | N(H)NMe₂ | O | O | CO₂H | — | — | 0 | N | methadone |
| B1-1479 | 4-(OH)Ph | O | O | CO₂H | — | — | 0 | N | methadone |
| B1-1480 | 4-(NH₂)Ph | O | O | CO₂H | — | — | 0 | N | methadone |
| B1-1481 | EtS | O | O | CO₂H | — | — | 0 | N | minoxidil |
| B1-1482 | EtO | O | O | CO₂H | — | — | 0 | N | minoxidil |
| B1-1483 | t-BuO | O | O | CO₂H | — | — | 0 | N | minoxidil |
| B1-1484 | cyclohexylO | O | O | CO₂H | — | — | 0 | N | minoxidil |
| B1-1485 | iPrO | O | O | CO₂H | — | — | 0 | N | minoxidil |
| B1-1486 | PhO | O | O | CO₂H | — | — | 0 | N | minoxidil |
| B1-1487 | (HOCH₂— | O | O | CO₂H | — | — | 0 | N | minoxidil |
| | CH₂)₂N | | | | | | | | |
| B1-1488 | N(H)NMe₂ | O | O | CO₂H | — | — | 0 | N | mininoxidil |
| B1-1489 | 4-(OH)Ph | O | O | CO₂H | — | — | 0 | N | minoxidil |
| B1-1490 | 4-(NH₂)Ph | O | O | CO₂H | — | — | 0 | N | minoxidil |
| B1-1491 | EtS | O | O | CO₂H | — | — | 0 | N | naftifine |
| B1-1492 | EtO | O | O | CO₂H | — | — | 0 | N | naftifine |
| B1-1493 | t-BuO | O | O | CO₂H | — | — | 0 | N | naftffine |
| B1-1494 | cyclohexylO | O | O | CO₂H | — | — | 0 | N | naftifine |
| B1-1495 | iPrO | O | O | CO₂H | — | — | 0 | N | naftifine |
| B1-1496 | PhO | O | O | CO₂H | — | — | 0 | N | naftifine |
| B1-1497 | (HOCH₂— | O | O | CO₂H | — | — | 0 | N | naftifine |
| | CH₂)₂N | | | | | | | | |
| B1-1498 | N(H)NMe₂ | O | O | CO₂H | — | — | 0 | N | naftifine |
| B1-1499 | 4-(OH)Ph | O | O | CO₂H | — | — | 0 | N | naftifine |
| B1-1500 | 4-(NH₂)Ph | O | O | CO₂H | — | — | 0 | N | naftifine |
| B1-1501 | EtS | O | O | CO₂H | — | — | 0 | N | orphenadrine |
| B1-1502 | EtO | O | O | CO₂H | — | — | 0 | N | orphenadrine |
| B1-1503 | t-BuO | O | O | CO₂H | — | — | 0 | N | orphenadrine |
| B1-1504 | cyclohexylO | O | O | CO₂H | — | — | 0 | N | orphenadrine |
| B1-1505 | iPrO | O | O | CO₂H | — | — | 0 | N | orphenadrine |
| B1-1506 | PhO | O | O | CO₂H | — | — | 0 | N | orphenadrine |
| B1-1507 | (HOCH₂— | O | O | CO₂H | — | — | 0 | N | orphenadrine |
| | CH₂)₂N | | | | | | | | |
| B1-1508 | N(H)NMe₂ | O | O | CO₂H | — | — | 0 | N | orphenadrine |
| B1-1509 | 4-(OH)Ph | O | O | CO₂H | — | — | 0 | N | orphenadrine |
| B1-1510 | 4-(NH₂)Ph | O | O | CO₂H | — | — | 0 | N | orphenadrine |
| B1-1511 | EtS | O | O | CO₂H | — | — | 0 | N | oxybutynin |
| B1-1512 | EtO | O | O | CO₂H | — | — | 0 | N | oxybutynin |
| B1-1513 | t-BuO | O | O | CO₂H | — | — | 0 | N | oxybutynin |
| B1-1514 | cyclohexylO | O | O | CO₂H | — | — | 0 | N | oxybutynin |
| B1-1515 | iPrO | O | O | CO₂H | — | — | 0 | N | oxybutynin |
| B1-1516 | PhO | O | O | CO₂H | — | — | 0 | N | oxybutynin |
| B1-1517 | (HOCH2— | O | O | CO₂H | — | — | 0 | N | oxybutynin |
| | CH₂)₂N | | | | | | | | |
| B1-1518 | N(H)NMe₂ | O | O | CO₂H | — | — | 0 | N | oxybutynin |
| B1-1519 | 4-(OH)Ph | O | O | CO₂H | — | — | 0 | N | oxybutynin |
| B1-1520 | 4-(NH₂)Ph | O | O | CO₂H | — | — | 0 | N | oxybutynin |
| B1-1521 | EtS | O | O | CO₂H | — | — | 0 | N | oxymetazoline |
| B1-1522 | EtO | O | O | CO₂H | — | — | 0 | N | oxymetazoline |
| B1-1523 | t-BuO | O | O | CO₂H | — | — | 0 | N | oxymetazoline |
| B1-1524 | cyclohexylO | O | O | CO₂H | — | — | 0 | N | oxymetazoline |
| B1-1525 | iPrO | O | O | CO₂H | — | — | 0 | N | oxymetazoline |
| B1-1526 | PhO | O | O | CO₂H | — | — | 0 | N | oxymetazoline |
| B1-1527 | (HOCH₂— | O | O | CO₂H | — | — | 0 | N | oxymetazoline |
| | CH₂)₂N | | | | | | | | |
| B1-1528 | N(H)NMe₂ | O | O | CO₂H | — | — | 0 | N | oxymetazoline |
| B1-1529 | 4-(OH)Ph | O | O | CO₂H | — | — | 0 | N | oxymetazoline |
| B1-1530 | 4-(NH₂)Ph | O | O | CO₂H | — | — | 0 | N | oxymetazoline |
| B1-1531 | EtS | O | O | CO₂H | — | — | 0 | N | phenoxybenzamine |
| B1-1532 | EtO | O | O | CO₂H | — | — | 0 | N | phenoxybenzamine |
| B1-1533 | t-BuO | O | O | CO₂H | — | — | 0 | N | phenoxybenzamine |
| B1-1534 | cyclohexylO | O | O | CO₂H | — | — | 0 | N | phenoxybenzamine |
| B1-1535 | iPrO | O | O | CO₂H | — | — | 0 | N | phenoxybenzamine |
| B1-1536 | PhO | O | O | CO₂H | — | — | 0 | N | phenoxybenzamine |
| B1-1537 | (HOCH₂— | O | O | COAl | — | — | 0 | N | phenoxybenzamine |
| | CH₂)₂N | | | | | | | | |
| B1-1538 | N(H)NMe₂ | O | O | CO₂H | — | — | 0 | N | phenoxybenzamine |
| B1-1539 | 4-(OH)Ph | O | O | CO₂H | — | — | 0 | N | phenoxybenzamine |
| B1-1540 | 4-(NH₂)Ph | O | O | CO₂H | — | — | 0 | N | phenoxybenzamine |
| B1-1541 | EtS | O | O | CO₂H | — | — | 0 | N | pilocarpine |
| B1-1542 | EtO | O | O | CO₂H | — | — | 0 | N | pilocarpine |
| B1-1543 | t-BuO | O | O | CO₂H | — | — | 0 | N | pilocarpine |
| B1-1544 | cyclohexylO | O | O | CO₂H | — | — | 0 | N | pilocarpine |
| B1-1545 | iPrO | O | O | CO₂H | — | — | 0 | N | pilocarpine |
| B1-1546 | PhO | O | O | CO₂H | — | — | 0 | N | pilocarpine |
| B1-1547 | (HOCH₂— | O | O | CO₂H | — | — | 0 | N | pilocarpine |
| | CH₂)₂N | | | | | | | | |
| B1-1548 | N(H)NMe₂ | O | O | CO₂H | — | — | 0 | N | pilocarpine |
| B1-1549 | 4-(OH)Ph | O | O | CO₂H | — | — | 0 | N | pilocarpine |
| B1-1550 | 4-(NH₂)Ph | O | O | CO₂H | — | — | 0 | N | pilocarpine |
| B1-1551 | EtS | O | O | CO₂H | — | — | 0 | N | pyrazinamide |
| B1-1552 | EtO | O | O | CO₂H | — | — | 0 | N | pyrazinamide |
| B1-1553 | t-BuO | O | O | CO₂H | — | — | 0 | N | pyrazinamide |
| B1-1554 | cyclohexylO | O | O | CO₂H | — | — | 0 | N | pyrazinamide |
| B1-1555 | iPrO | O | O | CO₂H | — | — | 0 | N | pyrazinamide |
| B1-1556 | PhO | O | O | CO₂H | — | — | 0 | N | pyrazinamide |
| B1-1557 | (HOCH₂— | O | O | CO₂H | — | — | 0 | N | pyrazinamide |
| | CH₂)₂N | | | | | | | | |
| B1-1558 | N(H)NMe₂ | O | O | CO₂H | — | — | 0 | N | pyrazinamide |
| B1-1559 | 4-(OH)Ph | O | O | CO₂H | — | — | 0 | N | pyrazinamide |
| B1-1560 | 4-(NH₂)Ph | O | O | CO₂H | — | — | 0 | N | pyrazinamide |
| B1-1561 | EtS | O | O | CO₂H | — | — | 0 | N | pyroxidine |
| B1-1562 | EtO | O | O | CO₂H | — | — | 0 | N | pyroxidine |
| B1-1563 | t-BuO | O | O | CO₂H | — | — | 0 | N | pyroxidine |
| B1-1564 | cyclohexylO | O | O | CO₂H | — | — | 0 | N | pyroxidine |
| B1-1565 | iPrO | O | O | CO₂H | — | — | 0 | N | pyroxidine |
| B1-1566 | PhO | O | O | CO₂H | — | — | 0 | N | pyroxidine |
| B1-1567 | (HOCH₂— | O | O | CO₂H | — | — | 0 | N | pyroxidine |
| | CH₂)₂N | | | | | | | | |
| B1-1568 | N(H)NMe₂ | O | O | CO₂H | — | — | 0 | N | pyroxidine |
| B1-1569 | 4-(OH)Ph | O | O | CO₂H | — | — | 0 | N | pyroxidine |
| B1-1570 | 4-(NH₂)Ph | O | O | CO₂H | — | — | 0 | N | pyroxidine |
| B1-1571 | EtS | O | O | CO₂H | — | — | 0 | N | risperidone |
| B1-1572 | EtO | O | O | CO₂H | — | — | 0 | N | risperidone |
| B1-1573 | t-BuO | O | O | CO₂H | — | — | 0 | N | risperidone |
| B1-1574 | cyclohexylO | O | O | CO₂H | — | — | 0 | N | risperidone |
| B1-1575 | iPrO | O | O | CO₂H | — | — | 0 | N | risperidone |
| B1-1576 | Ph0 | O | O | CO₂H | — | — | 0 | N | risperidone |
| B1-1577 | (HOCH₂— | O | O | CO₂H | — | — | 0 | N | risperidone |
| | CH₂)₂N | | | | | | | | |
| B1-1578 | N(H)NMe₂ | O | O | CO₂H | — | — | 0 | N | risperidone |
| B1-1579 | 4-(OH)Ph | O | O | CO₂H | — | — | 0 | N | risperidone |
| B1-1580 | 4-(NH₂)Ph | O | O | CO₂H | — | — | 0 | N | risperidone |
| B1-1581 | EtS | O | O | CO₂H | — | — | 0 | N | sufentanil |
| B1-1582 | EtO | O | O | CO₂H | — | — | 0 | N | sufentanil |
| B1-1583 | t-BuO | O | O | CO₂H | — | — | 0 | N | sufentanil |
| B1-1584 | cyclohexylO | O | O | CO₂H | — | — | 0 | N | sufentanil |
| B1-1585 | iPrO | O | O | CO₂H | — | — | 0 | N | sufentanil |
| B1-1586 | PhO | O | O | CO₂H | — | — | 0 | N | sufentanil |
| B1-1587 | (HOCH₂— | O | O | CO₂H | — | — | 0 | N | sufentanil |
| | CH₂)₂N | | | | | | | | |
| B1-1588 | N(H)NMe₂ | O | O | CO₂H | — | — | 0 | N | sufentanil |
| B1-1589 | 4-(OH)Ph | O | O | CO₂H | — | — | 0 | N | sufentanil |
| B1-1590 | 4-(NH_{2)Ph} | O | O | CO₂H | — | — | 0 | N | sufentanil |
| B1-1591 | EtS | O | O | CO₂H | — | — | 0 | N | tamoxifen |
| B1-1592 | EtO | O | O | CO₂H | — | — | 0 | N | tamoxifen |
| B1-1593 | t-BuO | O | O | CO₂H | — | — | 0 | N | tamoxifen |
| B1-1594 | cyclohexylO | O | O | CO₂H | — | — | 0 | N | tamoxifen |
| B1-1595 | iPrO | O | O | CO₂H | — | — | 0 | N | tamoxifen |
| B1-1596 | PhO | O | O | CO₂H | — | — | 0 | N | tamoxifen |
| B1-1597 | (HOCH₂— | O | O | CO₂H | — | — | 0 | N | tamoxifen |
| | CH₂)₂N | | | | | | | | |
| B1-1598 | N(H)NMe₂ | O | O | CO₂H | — | — | 0 | N | tamoxifen |
| B1-1599 | 4-(OH)Ph | O | O | CO₂H | — | — | 0 | N | tamoxifen |
| B1-1600 | 4-(NH₂)Ph | O | O | CO₂H | — | — | 0 | N | tamoxifen |
| B1-1601 | EtS | O | O | CO₂H | — | — | 0 | N | terbinafine |
| B1-1602 | EtO | O | O | CO₂H | — | — | 0 | N | terbinafine |
| B1-1603 | t-BuO | O | O | CO₂H | — | — | 0 | N | terbinafine |
| B1-1604 | cyclohexylO | O | O | CO₂H | — | — | 0 | N | terbinafine |
| B1-1605 | iPrO | O | O | CO₂H | — | — | 0 | N | terbinafine |
| B1-1606 | PhO | O | O | CO₂H | — | — | 0 | N | terbinafine |
| B1-1607 | (HOCH₂— | O | O | CO₂H | — | — | 0 | N | terbinafine |
| | CH₂)₂N | | | | | | | | |
| B1-1608 | N(H)NMe₂ | O | O | CO₂H | — | — | 0 | N | terbinafine |
| B1-1609 | 4-(OH)Ph | O | O | CO₂H | — | — | 0 | N | terbinafine |
| B1-1610 | 4-(NH₂)Ph | O | O | CO₂H | — | — | 0 | N | terbinafine |
| B1-1611 | EtS | O | O | CO₂H | — | — | 0 | N | trihexyphenidyl |
| B1-1612 | EtO | O | O | CO₂H | — | — | 0 | N | trihexyphenidyl |
| B1-1613 | t-BuO | O | O | CO₂H | — | — | 0 | N | trihexyphenidyl |
| B1-1614 | cyclohexylO | O | O | CO₂H | — | — | 0 | N | trihexyphenidyl |
| B1-1615 | iPrO | O | O | CO₂H | — | — | 0 | N | trihexyphenidyl |
| B1-1616 | PhO | O | O | CO₂H | — | — | 0 | N | trihexyphenidyl |
| B1-1617 | (HOCH₂— | O | O | CO₂H | — | — | 0 | N | trihexyphenidyl |
| | CH₂)₂N | | | | | | | | |
| B1-1618 | N(H)NMe₂ | O | O | CO₂H | — | — | 0 | N | trihexyphenidyl |
| B1-1619 | 4-(OH)Ph | O | O | CO₂H | — | — | 0 | N | trihexyphenidyl |
| B1-1620 | 4-(NH₂)Ph | O | O | CO₂H | — | — | 0 | N | trihexyphenidyl |
| B1-1621 | EtS | O | O | CO₂H | — | — | 0 | N | troleandomycin |
| B1-1622 | EtO | O | O | CO₂H | — | — | 0 | N | troleandomycin |
| B1-1623 | t-BuO | O | O | CO₂H | — | — | 0 | N | troleandomycin |
| Bl-1624 | cyclohexylO | O | O | CO₂H | — | — | 0 | N | troleandomycin |
| B1-1625 | iPr0 | O | O | CO₂H | — | — | 0 | N | troleandomycin |
| B1-1626 | PhO | O | O | CO₂H | — | — | 0 | N | troleandomycin |
| B1-1627 | (HOCH₂— | O | O | CO₂H | — | — | 0 | N | troleandomycin |
| | CH₂)₂N | | | | | | | | |
| B1-1628 | N(H)NMe₂ | O | O | CO₂H | — | — | 0 | N | troleandomycin |
| B1-1629 | 4-(OH)Ph | O | O | CO₂H | — | — | 0 | N | troleandomycin |
| B1-1630 | 4-(NH₂)Ph | O | O | CO₂H | — | — | 0 | N | troleandomycin |
| B1-1631 | EtS | O | O | CO₂H | — | — | 0 | N | verapamil |
| B1-1632 | EtO | O | O | CO₂H | — | — | 0 | N | verapamil |
| B1-1633 | t-BuO | O | O | CO₂H | — | — | 0 | N | verapamil |
| B1-1634 | cyclohexylO | O | O | CO₂H | — | — | 0 | N | verapamil |
| B1-1635 | iPrO | O | O | CO₂H | — | — | 0 | N | verapamil |
| B1-1636 | PhO | O | O | CO₂H | — | — | 0 | N | verapamil |
| B1-1637 | (HOCH₂— | O | O | CO₂H | — | — | 0 | N | verapamil |
| | CH₂)₂N | | | | | | | | |
| B1-1638 | N(H)NMe₂ | O | O | CO₂H | — | — | 0 | N | verapamil |
| B1-1639 | 4-(OH)Ph | O | O | CO₂H | — | — | 0 | N | verapamil |
| B1-1640 | 4-(NH₂)Ph | O | O | CO₂H | — | — | 0 | N | verapamil |
| B1-1641 | EtS | O | O | CO₂H | — | — | 0 | N | caffeine |
| B1-1642 | EtO | O | O | CO₂H | — | — | 0 | N | caffeine |
| B1-1643 | t-BuO | O | O | CO₂H | — | — | 0 | N | caffeine |
| B1-1644 | cyclohexylO | O | O | CO₂H | — | — | 0 | N | caffeine |
| B1-1645 | iPrO | O | O | CO₂H | — | — | 0 | N | caffeine |
| B1-1646 | PhO | O | O | CO₂H | — | — | 0 | N | caffeine |
| B1-1647 | (HOCH₂— | O | O | CO₂H | — | — | 0 | N | caffeine |
| | CH₂)₂N | | | | | | | | |
| B1-1648 | N(H)NMe₂ | O | O | CO₂H | — | — | 0 | N | caffeine |
| B1-1649 | 4-(OH)Ph | O | O | CO₂H | — | — | 0 | N | caffeine |
| B1-1650 | 4-(NH₂)Ph | O | O | CO₂H | — | — | 0 | N | caffeine |
| B1-1651 | EtS | O | O | CO₂H | — | — | 0 | N | cyproheptadine |
| B1-1652 | EtO | O | O | CO₂H | — | — | 0 | N | cyproheptadine |
| B1-1653 | t-BuO | O | O | CO₂H | — | — | 0 | N | cyproheptadine |
| B1-1654 | cyclohexylO | O | O | CO₂H | — | — | 0 | N | cyproheptadine |
| B1-1655 | iPrO | O | O | CO₂H | — | — | 0 | N | cyproheptadine |
| B1-1656 | PhO | O | O | CO₂H | — | — | 0 | N | cyproheptadine |
| B1-1657 | (HOCH₂— | O | O | CO₂H | — | — | 0 | N | cyproheptadine |
| | CH₂)₂N | | | | | | | | |
| B1-1658 | N(H)NMe₂ | O | O | CO₂H | — | — | 0 | N | cyproheptadine |
| B1-1659 | 4-(OH)Ph | O | O | CO₂H | — | — | 0 | N | cyproheptadine |
| B1-1660 | 4-(NH₂)Ph | O | O | CO₂H | — | — | 0 | N | cyproheptadine |
| B1-1661 | EtS | O | O | CO₂H | — | — | 0 | N | pramoxine |
| B1-1662 | EtO | O | O | CO₂H | — | — | 0 | N | pramoxine |
| B1-1663 | t-BuO | O | O | CO₂H | — | — | 0 | N | pramoxine |
| B1-1664 | cyclohexylO | O | O | CO₂H | — | — | 0 | N | pramoxine |
| B1-1665 | iPrO | O | 0 | CO₂H | — | — | 0 | N | pramoxine |
| B1-1666 | PhO | O | O | CO₂H | — | — | 0 | N | pramoxine |
| B1-1667 | (HOCH₂— | O | O | CO₂H | — | — | 0 | N | pramoxine |
| | CH₂)₂N | | | | | | | | |
| B1-1668 | N(H)NMe₂ | O | O | CO₂H | — | — | 0 | N | pramoxine |
| B1-1669 | 4-(OH)Ph | O | O | CO₂H | — | — | 0 | N | pramoxine |
| B1-1670 | 4-(NH₂)Ph | O | O | CO₂H | — | — | 0 | N | pramoxine |
| B1-1671 | EtS | O | O | CO₂H | — | — | 0 | O | iodoquinol |
| B1-1672 | EtO | O | O | CO₂H | — | — | 0 | O | iodoquinol |
| B1-1673 | t-BuO | O | O | CO₂H | — | — | 0 | O | iodoquinol |
| B1-1674 | cyclohexylO | O | O | CO₂H | — | — | 0 | O | iodoquinol |
| B1-1675 | iPrO | O | O | CO₂H | — | — | 0 | O | iodoquinol |
| B1-1676 | PhO | O | O | CO₂H | — | — | 0 | O | iodoquinol |
| B1-1677 | (HOCH₂— | O | O | CO₂H | — | — | 0 | O | iodoquinol |
| | CH₂)₂N | | | | | | | | |
| B1-1678 | N(H)NMe₂ | O | O | CO₂H | — | — | 0 | O | iodoquinol |
| B1-1679 | 4-(OH)Ph | O | O | CO₂H | — | — | 0 | O | iodoquinol |
| B1-1680 | 4-(NH₂)Ph | O | O | CO₂H | — | — | 0 | O | iodoquinol |
| B1-1681 | EtS | O | O | CO₂H | — | — | 0 | O | metronidazole |
| B1-1682 | EtO | O | O | CO₂H | — | — | 0 | O | metronidazole |
| B1-1683 | t-BuO | O | O | CO₂H | — | — | 0 | O | metronidazole |
| B1-1684 | cyclohexylO | O | O | CO₂H | — | — | 0 | O | metronidazole |
| B1-1685 | iPrO | O | O | CO₂H | — | — | 0 | O | metronidazole |
| B1-1686 | PhO | O | O | CO₂H | — | — | 0 | O | metronidazole |
| B1-1687 | (HOCH₂— | O | O | CO₂H | — | — | 0 | O | metronidazole |
| | CH₂)₂N | | | | | | | | |
| B1-1688 | N(H)NMe₂ | O | O | CO₂H | — | — | 0 | O | metronidazole |
| B1-1689 | 4-(OH)Ph | O | O | CO₂H | — | — | 0 | O | metronidazole |
| B1-1690 | 4-(NH₂)Ph | O | O | CO₂H | — | — | 0 | O | metronidazole |
| B1-1691 | EtS | O | O | CO₂H | — | — | 0 | N | papaverine |
| B1-1692 | EtO | O | O | CO₂H | — | — | 0 | N | papaverine |
| B1-1693 | t-BuO | O | O | CO₂H | — | — | 0 | N | papaverine |
| B1-1694 | cyclohexylO | O | O | CO₂H | — | — | 0 | N | papaverine |
| B1-1695 | iPrO | O | O | CO₂H | — | — | 0 | N | papaverme |
| B1-1696 | PhO | O | O | CO₂H | — | — | 0 | N | papaverine |
| B1-1697 | (HOCH₂— | O | O | CO₂H | — | — | 0 | N | papaverine |
| | CH₂)₂N | | | | | | | | |
| B1-1698 | N(H)NMe₂ | O | O | CO₂H | — | — | 0 | N | papaverine |
| B1-1699 | 4-(OH)Ph | O | O | CO₂H | — | — | 0 | N | papaverine |
| B1-1700 | 4-(NH₂)Ph | O | O | CO₂H | — | — | 0 | N | papaverine |
| B1-1701 | EtS | O | O | CO₂H | — | — | 0 | N | tropicamide |
| B1-1702 | EtO | O | O | CO₂H | — | — | 0 | N | tropicamide |
| B1-1703 | t-BuO | O | O | CO₂H | — | — | 0 | N | tropicamide |
| B1-1704 | cyclohexylO | O | O | CO₂H | — | — | 0 | N | tropicamide |
| B1-1705 | iPrO | O | O | CO₂H | — | — | 0 | N | tropicamide |
| B1-1706 | PhO | O | O | CO₂H | — | — | 0 | N | tropicamide |
| B1-1707 | (HOCH₂— | O | O | CO₂H | — | — | 0 | N | tropicamide |
| | CH₂)₂N | | | | | | | | |
| B1-1708 | N(H)NMe₂ | O | O | CO₂H | — | — | 0 | N | tropicamide |
| B1-1709 | 4-(OH)Ph | O | O | CO₂H | — | — | 0 | N | tropicamide |
| B1-1710 | 4-(NH₂)Ph | O | O | CO₂H | — | — | 0 | N | tropicamide |
| B1-1711 | EtS | O | O | CO₂H | — | — | 0 | N | halazepam |
| B1-1712 | EtO | O | O | CO₂H | — | — | 0 | N | halazep am |
| B1-1713 | t-BuO | O | O | CO₂H | — | — | 0 | N | halazepam |
| B1-1714 | cyclohexylO | O | O | CO₂H | — | — | 0 | N | halazepam |
| B1-1715 | iPrO | O | O | CO₂H | — | — | 0 | N | halazepam |
| B1-1716 | PhO | O | O | CO₂H | — | — | 0 | N | halazepam |
| B1-1717 | (HOCH₂— | O | O | CO₂H | — | — | 0 | N | halazepam |
| | CH₂)₂N | | | | | | | | |
| B1-1718 | N(H)NMe₂ | O | O | CO₂H | — | — | 0 | N | halazepam |
| B1-1719 | 4-(OH)Ph | O | O | CO₂H | — | — | 0 | N | halazepam |
| B1-1720 | 4-(NH₂)Ph | O | O | CO₂H | — | — | 0 | N | halazepam |
| B1-1721 | EtS | O | O | CO₂H | — | — | 0 | O | mazindol |
| B1-1722 | EtO | O | O | CO₂H | — | — | 0 | O | mazindol |
| B1-1723 | t-BuO | O | O | CO₂H | — | — | 0 | O | mazindol |
| B1-1724 | cyclohexylO | O | O | CO₂H | — | — | 0 | O | mazindol |
| B1-1725 | iPrO | O | O | CO₂H | — | — | 0 | O | mazindol |
| B1-1726 | PhO | O | O | CO₂H | — | — | 0 | O | mazindol |
| B1-1727 | (HOCH₂— | O | O | CO₂H | — | — | 0 | O | mazindol |
| | CH₂)₂N | | | | | | | | |
| B1-1728 | N(H)NMe₂ | O | O | CO₂H | — | — | 0 | O | mazindol |
| B1-1729 | 4-(OH)Ph | O | O | CO₂H | — | — | 0 | O | mazindol |
| B1-1730 | 4-(NH₂)Ph | O | O | CO₂H | — | — | 0 | O | mazindol |
| B1-1731 | EtS | O | O | CO₂H | — | — | 0 | O | hydroxyitraconazole |
| B1-1732 | EtO | O | O | CO₂H | — | — | 0 | O | hydroxyitraconazole |
| B1-1733 | t-BuO | O | O | CO₂H | — | — | 0 | O | hydroxyitraconazole |
| B1-1734 | cyclohexylO | O | O | CO₂H | — | — | 0 | O | hydroxyitraconazole |
| B1-1735 | iPrO | O | O | CO₂H | — | — | 0 | O | hydroxyitraconazole |
| B1-1736 | PhO | O | O | CO₂H | — | — | 0 | O | hydroxyitraconazole |
| B1-1737 | (HOCH₂— | O | O | CO₂H | — | — | 0 | O | hydroxyitraconazole |
| | CH₂)₂N | | | | | | | | |
| B1-1738 | N(H)NMe₂ | O | O | CO₂H | — | — | 0 | O | hydroxyitraconazole |
| B1-1739 | 4-(OH)Ph | O | O | CO₂H | — | — | 0 | O | hydroxyitraconazole |
| B1-1740 | 4-(NH₂)Ph | O | O | CO₂H | — | — | 0 | O | hydroxyitraconazole |
| B1-1741 | EtS | O | O | CO₂H | — | — | 0 | O | posaconazole |
| B1-1742 | EtO | O | O | CO₂H | — | — | 0 | O | posaconazole |
| B1-1743 | t-BuO | O | O | CO₂H | — | — | 0 | O | posaconazole |
| B1-1744 | cyclohexylO | O | O | CO₂H | — | — | 0 | O | posaconazole |
| B1-1745 | iPrO | O | O | CO₂H | — | — | 0 | O | posaconazole |
| B1-1746 | PhO | O | O | CO₂H | — | — | 0 | O | posaconazole |
| B1-1747 | (HOCH₂— | O | O | CO₂H | — | — | 0 | O | posaconazole |
| | CH₂)₂N | | | | | | | | |
| B1-1748 | N(H)NMe₂ | O | O | CO₂H | — | — | 0 | O | posaconazole |
| B1-1749 | 4-(OH)Ph | O | O | CO₂H | — | — | 0 | O | posaconazole |
| B1-1750 | 4-(NH₂)Ph | O | O | CO₂H | — | — | 0 | O | posaconazole |
| B1-1751 | EtS | O | O | CO₂H | — | — | 0 | O | voriconazole |
| B1-1752 | EtO | O | O | CO₂H | — | — | 0 | O | voriconazole |
| B1-1753 | t-BuO | O | O | CO₂H | — | — | 0 | O | voriconazole |
| B1-1754 | cyclohexylO | O | O | CO₂H | — | — | 0 | O | voriconazole |
| B1-1755 | iPrO | O | O | CO₂H | — | — | 0 | O | voriconazole |
| B1-1756 | PhO | O | O | CO₂H | — | — | 0 | O | voriconazole |
| B1-1757 | (HOCH₂— | O | O | CO₂H | — | — | 0 | O | voriconazole |
| | CH₂)₂N | | | | | | | | |
| B1-1758 | N(H)NMe₂ | O | O | CO₂H | — | — | 0 | O | voriconazole |
| B1-1759 | 4-(OH)Ph | O | O | CO₂H | — | — | 0 | O | voriconazole |
| B1-1760 | 4-(NH₂)Ph | O | O | CO₂H | — | — | 0 | O | voriconazole |
| B1-1761 | EtS | O | O | CO₂H | — | — | 0 | O | fluconazole |
| B1-1762 | EtO | O | O | CO₂H | — | — | 0 | O | fluconazole |
| B1-1763 | t-BuO | O | O | CO₂H | — | — | 0 | O | fluconazole |
| B1-1764 | cyclohexylO | O | O | CO₂H | — | — | 0 | O | fluconazole |
| B1-1765 | iPrO | O | O | CO₂H | — | — | 0 | O | fluconazole |
| B1-1766 | PhO | O | O | CO₂H | — | — | 0 | O | fluconazole |
| B1-1767 | (HOCH₂— | O | O | CO₂H | — | — | 0 | O | fluconazole |
| | CH₂)₂N | | | | | | | | |
| B1-1768 | N(H)NMe₂ | O | O | CO₂H | — | — | 0 | O | fluconazole |
| B1-1769 | 4-(OH)Ph | O | O | CO₂H | — | — | 0 | O | fluconazole |
| B1-1770 | 4-(NH₂)Ph | O | O | CO₂H | — | — | 0 | O | fluconazole |
| B1-1771 | EtS | O | O | CO₂H | — | — | 0 | O | genaconazole |
| B1-1772 | EtO | O | O | CO₂H | — | — | 0 | O | genaconazole |
| B1-1773 | t-BuO | O | O | CO₂H | — | — | 0 | O | genaconazole |
| B1-1774 | cyclohexylO | O | O | CO₂H | — | — | 0 | O | genaconazole |
| B1-1775 | iPrO | O | O | CO₂H | — | — | 0 | O | genaconazole |
| B1-1776 | PhO | O | O | CO₂H | — | — | 0 | O | genaconazole |
| B1-1777 | (HOCH₂— | O | O | CO₂H | — | — | 0 | O | genaconazole |
| | CH₂)₂N | | | | | | | | |
| B1-1778 | N(H)NMe₂ | O | O | CO₂H | — | — | 0 | O | genaconazole |
| B1-1779 | 4-(OH)Ph | O | O | CO₂H | — | — | 0 | O | genaconazole |
| B1-1780 | 4-(NH₂)Ph | O | O | CO₂H | — | — | 0 | O | genaconazole |
| B1-1781 | EtS | O | O | CO₂H | — | — | 0 | N | aliconazole |
| B1-1782 | EtO | O | O | CO₂H | — | — | 0 | N | aliconazole |
| B1-1783 | t-BuO | O | O | CO₂H | — | — | 0 | N | aliconazole |
| B1-1784 | cyclohexylO | O | O | CO₂H | — | — | 0 | N | aliconazole |
| B1-1785 | iPrO | O | O | CO₂H | — | — | 0 | N | aliconazole |
| B1-1786 | PhO | O | O | CO₂H | — | — | 0 | N | aliconazole |
| B1-1787 | (HOCH₂— | O | O | CO₂H | — | — | 0 | N | aliconazole |
| | CH₂)₂N | | | | | | | | |
| B1-1788 | N(H)NMe₂ | O | O | CO₂H | — | — | 0 | N | aliconazole |
| B1-1789 | 4-(OH)Ph | O | O | CO₂H | — | — | 0 | N | aliconazole |
| B1-1790 | 4-(NH₂)Ph | O | O | CO₂H | — | — | 0 | N | aliconazole |
| B1-1791 | EtS | O | O | CO₂H | — | — | 0 | N | beciconazole |
| B1-1792 | EtO | O | O | CO₂H | — | — | 0 | N | becliconazole |
| B1-1793 | t-BuO | O | O | CO₂H | — | — | 0 | N | beciconazole |
| B1-1794 | cyclohexylO | O | O | CO₂H | — | — | 0 | N | becliconazole |
| B1-1795 | iPrO | O | O | CO₂H | — | — | 0 | N | becliconazole |
| B1-1796 | PhO | O | O | CO₂H | — | — | 0 | N | beciconazole |
| B1-1797 | (HOCH₂— | O | O | CO₂H | — | — | 0 | N | beciconazole |
| | CH₂)₂N | | | | | | | | |
| B1-1798 | N(H)NMe₂ | O | O | CO₂H | — | — | 0 | N | beciconazole |
| B1-1799 | 4-(OH)Ph | O | O | CO₂H | — | — | 0 | N | becliconazole |
| B1-1800 | 4-(NH₂)Ph | O | O | CO₂H | — | — | 0 | N | becliconazole |
| B1-1801 | EtS | O | O | CO₂H | — | — | 0 | N | brolaconazole |
| B1-1802 | EtO | O | O | CO₂H | — | — | 0 | N | brolaconazole |
| B1-1803 | t-BuO | O | O | CO₂H | — | — | 0 | N | brolaconazole |
| B1-1804 | cyclohexylO | O | O | CO₂H | — | — | 0 | N | brolaconazole |
| B1-1805 | iPrO | O | O | CO₂H | — | — | 0 | N | brolaconazole |
| B1-1806 | PhO | O | O | CO₂H | — | — | 0 | N | brolaconazole |
| B1-1807 | (HOCH₂— | O | O | CO₂H | — | — | 0 | N | brolaconazole |
| | CH₂)₂N | | | | | | | | |
| B1-1808 | N(H)NMe₂ | O | O | CO₂H | — | — | 0 | N | brolaconazole |
| B1-1809 | 4-(OH)Ph | O | O | CO₂H | — | — | 0 | N | brolaconazole |
| B1-1810 | 4-(NH₂)Ph | O | O | CO₂H | — | — | 0 | N | brolaconazole |
| B1-1811 | EtS | O | O | CO₂H | — | — | 0 | N | butaconazole |
| B1-1812 | EtO | O | O | CO₂H | — | — | 0 | N | butaconazole |
| B1-1813 | t-BuO | O | O | CO₂H | — | — | 0 | N | butaconazole |
| B1-1814 | cyclohexylO | O | O | CO₂H | — | — | 0 | N | butaconazole |
| B1-1815 | iPrO | O | O | CO₂H | — | — | 0 | N | butaconazole |
| B1-1816 | PhO | O | O | CO₂H | — | — | 0 | N | butaconazole |
| B1-1817 | (HOCH₂— | O | O | CO₂H | — | — | 0 | N | butaconazole |
| | CH₂)₂N | | | | | | | | |
| B1-1818 | N(H)NMe₂ | O | O | CO₂H | — | — | 0 | N | butaconazole |
| B1-1819 | 4-(OH)Ph | O | O | CO₂H | — | — | 0 | N | butaconazole |
| B1-1820 | 4-(NH₂)Ph | O | O | CO₂H | — | — | 0 | N | butaconazole |
| B1-1821 | EtS | O | O | CO₂H | — | — | 0 | N | clotrimazole |
| B1-1822 | EtO | O | O | CO₂H | — | — | 0 | N | clotrimazole |
| B1-1823 | t-BuO | O | O | CO₂H | — | — | 0 | N | clotrimazole |
| B1-1824 | cyclohexylO | O | O | CO₂H | — | — | 0 | N | clotrimazole |
| B1-1825 | iPrO | O | O | CO₂H | — | — | 0 | N | clotrimazole |
| B1-1826 | PhO | O | O | CO₂H | — | — | 0 | N | clotrimazole |
| B1-1827 | (HOCH₂— | O | O | CO₂H | — | — | 0 | N | clotrimazole |
| | CH₂)₂N | | | | | | | | |
| B1-1828 | N(H)NMe₂ | O | O | CO₂H | — | — | 0 | N | clotrimazole |
| B1-1829 | 4-(OH)Ph | O | O | CO₂H | — | — | 0 | N | clotrimazole |
| B1-1830 | 4-(NH₂)Ph | O | O | CO₂H | — | — | 0 | N | clotrimazole |
| B1-1831 | EtS | O | O | CO₂H | — | — | 0 | N | croconazole |
| B1-1832 | EtO | O | O | CO₂H | — | — | 0 | N | croconazole |
| B1-1833 | t-BuO | O | O | CO₂H | — | — | 0 | N | croconazole |
| B1-1834 | cyclohexylO | O | O | CO₂H | — | — | 0 | N | croconazole |
| B1-1835 | iPrO | O | O | CO₂H | — | — | 0 | N | croconazole |
| B1-1836 | PhO | O | O | CO₂H | — | — | 0 | N | croconazole |
| B1-1837 | (HOCH₂— | O | O | CO₂H | — | — | 0 | N | croconazole |
| | CH₂)₂N | | | | | | | | |
| B1-1838 | N(H)NMe₂ | O | O | CO₂H | — | — | 0 | N | croconazole |
| B1-1839 | 4-(OH)Ph | O | O | CO₂H | — | — | 0 | N | croconazole |
| B1-1840 | 4-(NH₂)Ph | O | O | CO₂H | — | — | 0 | N | croconazole |
| B1-1841 | EtS | O | O | CO₂H | — | — | 0 | N | econazole |
| B1-1842 | EtO | O | O | CO₂H | — | — | 0 | N | econazole |
| B1-1843 | t-BuO | O | O | CO₂H | — | — | 0 | N | econazole |
| B1-1844 | cyclohexylO | O | O | CO₂H | — | — | 0 | N | econazole |
| B1-1845 | iPrO | O | O | CO₂H | — | — | 0 | N | econazole |
| B1-1846 | PhO | O | O | COdI | — | — | 0 | N | econazole |
| B1-1847 | (HOCH_{2—} | O | O | CO₂H | — | — | 0 | N | econazole |
| | CH₂)₂N | | | | | | | | |
| B1-1848 | N(H)NMe₂ | O | O | CO₂H | — | — | 0 | N | econazole |
| B1-1849 | 4-(OH)Ph | O | O | CO₂H | — | — | 0 | N | econazole |
| B1-1850 | 4-(NH₂)Ph | O | O | CO₂H | — | — | 0 | N | econazole |
| B1-1851 | EtS | O | O | CO₂H | — | — | 0 | N | democonazole |
| B1-1852 | EtO | O | O | CO₂H | — | — | 0 | N | democonazole |
| B1-1853 | t-BuO | O | O | CO₂H | — | — | 0 | N | democonazole |
| B1-1854 | cyclohexylO | O | O | CO₂H | — | — | 0 | N | democonazole |
| B1-1855 | iPrO | O | O | CO₂H | — | — | 0 | N | democonazole |
| B1-1856 | PhO | O | O | CO₂H | — | — | 0 | N | democonazole |
| B1-1857 | (HOCH₂— | O | O | CO₂H | — | — | 0 | N | democonazole |
| | CH₂)₂N | | | | | | | | |
| B1-1858 | N(H)NMe₂ | O | O | CO₂H | — | — | 0 | N | democonazole |
| B1-1859 | 4-(OH)Ph | O | O | CO₂H | — | — | 0 | N | democonazole |
| B1-1860 | 4-(NH₂)Ph | O | O | CO₂H | — | — | 0 | N | democonazole |
| B1-1861 | EtS | O | O | CO₂H | — | — | 0 | N | doconazole |
| B1-1862 | EtO | O | O | CO₂H | — | — | 0 | N | doconazole |
| B1-1863 | t-BuO | O | O | CO₂H | — | — | 0 | N | doconazole |
| B1-1864 | cyclohexylO | O | O | CO₂H | — | — | 0 | N | doconazole |
| B1-1865 | iPrO | O | O | CO₂H | — | — | 0 | N | doconazole |
| B1-1866 | PhO | O | O | CO₂H | — | — | 0 | N | doconazole |
| B1-1867 | (HOCH₂— | O | O | CO₂H | — | — | 0 | N | doconazole |
| | CH₂)₂N | | | | | | | | |
| B1-1868 | N(H)NMe₂ | O | O | CO₂H | — | — | 0 | N | doconazole |
| B1-1869 | 4-(OH)Ph | O | O | CO₂H | — | — | 0 | N | doconazole |
| B1-1870 | 4-(NH₂)Ph | O | O | CO₂H | — | — | 0 | N | doconazole |
| B1-1871 | EtS | O | O | CO₂H | — | — | 0 | N | fenticonazole |
| B1-1872 | EtO | O | O | CO₂H | — | — | 0 | N | fenticonazole |
| B1-1873 | t-BuO | O | O | CO₂H | — | — | 0 | N | fenticonazole |
| B1-1874 | cyclohexylO | O | O | CO₂H | — | — | 0 | N | fenticonazole |
| B1- 1875 | iPrO | O | O | CO₂H | — | — | 0 | N | fenticonazole |
| B1-1876 | PhO | O | O | CO₂H | — | — | 0 | N | fenticonazole |
| B1-1877 | (HOCH₂— | O | O | CO₂H | — | — | 0 | N | fenticonazole |
| | CH₂)₂N | | | | | | | | |
| B1-1878 | N(H)NMe₂ | O | O | CO₂H | — | — | 0 | N | fenticonazole |
| B1-1879 | 4-(OH)Ph | O | O | CO₂H | — | — | 0 | N | fenticonazole |
| B1-1880 | 4-(NH₂)Ph | O | O | CO₂H | — | — | 0 | N | fenticonazole |
| B1-1881 | EtS | O | O | CO₂H | — | — | 0 | N | eberconazole |
| B1-1882 | EtO | O | O | CO₂H | — | — | 0 | N | eberconazole |
| B1-1883 | t-BuO | O | O | CO₂H | — | — | 0 | N | eberconazole |
| B1-1884 | cyclohexylO | O | O | CO₂H | — | — | 0 | N | eberconazole |
| B1-1885 | iPrO | O | O | CO₂H | — | — | 0 | N | eberconazole |
| B1-1886 | PhO | O | O | CO₂H | — | — | 0 | N | eberconazole |
| B1-1887 | (HOCH₂— | O | O | CO₂H | — | — | 0 | N | eberconazole |
| | CH₂)₂N | | | | | | | | |
| B1-1888 | N(H)NMe₂ | O | O | CO₂H | — | — | 0 | N | eberconazole |
| B1-1889 | 4-(OH)Ph | O | O | CO₂H | — | — | 0 | N | eberconazole |
| B1-1890 | 4-(NH₂)Ph | O | O | CO₂H | — | — | 0 | N | eberconazole |
| B1-1891 | EtS | O | O | CO₂H | — | — | 0 | N | isoconazole |
| B1-1892 | EtO | O | 0 | CO₂H | — | — | 0 | N | isoconazole |
| B1-1893 | t-BuO | O | O | CO₂H | — | — | 0 | N | isoconazole |
| B1-1894 | cyclohexylO | O | O | CO₂H | — | — | 0 | N | isoconazole |
| B1-1895 | iPrO | O | O | CO₂H | — | — | 0 | N | isoconazole |
| B1-1896 | PhO | O | O | CO₂H | — | — | 0 | N | isoconazole |
| B1-1897 | (HOCH₂— | O | O | CO₂H | — | — | 0 | N | isoconazole |
| | CH₂)₂N | | | | | | | | |
| B1-1898 | N(H)NMe₂ | O | O | CO₂H | — | — | 0 | N | isoconazole |
| B1-1899 | 4-(OH)Ph | O | O | CO₂H | — | — | 0 | N | isoconazole |
| B1-1900 | 4-(NH2)Ph | O | O | CO₂H | — | — | 0 | N | isoconazole |
| B1-1901 | EtS | O | O | CO₂H | — | — | 0 | N | miconazole |
| B1-1902 | EtO | O | O | CO₂H | — | — | 0 | N | miconazole |
| B1-1903 | t-BuO | O | O | CO₂H | — | — | 0 | N | miconazole |
| B1-1904 | cyclohexylO | O | O | CO₂H | — | — | 0 | N | miconazole |
| B1-1905 | iPrO | O | O | CO₂H | — | — | 0 | N | miconazole |
| B1-1906 | PhO | O | O | CO₂H | — | — | 0 | N | miconazole |
| B1-1907 | (HOCH₂— | O | O | CO₂H | — | — | 0 | N | miconazole |
| | CH₂)₂N | | | | | | | | |
| B1-1908 | N(H)NMe₂ | O | O | CO₂H | — | — | 0 | N | miconazole |
| B1-1909 | 4-(OH)Ph | O | O | CO₂H | — | — | 0 | N | miconazole |
| B1-1910 | 4-(NH₂)Ph | O | O | CO₂H | — | — | 0 | N | miconazole |
| B1-1911 | EtS | O | O | CO₂H | — | — | 0 | N | neticonazole |
| B1-1912 | EtO | O | O | CO₂H | — | — | 0 | N | neticonazole |
| B1-1913 | t-BuO | O | O | CO₂H | — | — | 0 | N | neticonazok |
| B1-1914 | cyclohexylO | O | O | CO₂H | — | — | 0 | N | neticonazole |
| B1-1915 | iPrO | O | O | CO₂H | — | — | 0 | N | neticonazole |
| B1-1916 | PhO | O | O | CO₂H | — | — | 0 | N | neticonazole |
| B1-1917 | (HOCH₂— | O | O | CO₂H | — | — | 0 | N | neticonazole |
| | CH₂)₂N | | | | | | | | |
| B1-1918 | N(H)NMe₂ | O | O | CO₂H | — | — | 0 | N | neticonazole |
| B1-1919 | 4-(OH)Ph | O | O | CO₂H | — | — | 0 | N | neticonazole |
| B1-1920 | 4-(NH₂)Ph | O | O | CO₂H | — | — | 0 | N | neticonazole |
| B1-1921 | EtS | O | O | CO₂H | — | — | 0 | N | omoconazole |
| B1-1922 | EtO | O | O | CO₂H | — | — | 0 | N | omoconazole |
| B1-1923 | t-BuO | O | O | CO₂H | — | — | 0 | N | omoconazole |
| B1-1924 | cyclohexylO | O | O | CO₂H | — | — | 0 | N | omoconazole |
| B1-1925 | iPrO | O | O | CO₂H | — | — | 0 | N | omoconazole |
| B1-1926 | PhO | O | O | CO₂H | — | — | 0 | N | omoconazole |
| B1-1927 | (HOCH₂— | O | O | CO₂H | — | — | 0 | N | omoconazole |
| | CH₂)₂N | | | | | | | | |
| B1-1928 | N(H)NMe₂ | O | O | CO₂H | — | — | 0 | N | omoconazole |
| B1-1929 | 4-(OH)Ph | O | O | CO₂H | — | — | 0 | N | omoconazole |
| B1-1930 | 4-(NH₂)Ph | O | O | CO₂H | — | — | 0 | N | omoconazole |
| B1-1931 | EtS | O | O | CO₂H | — | — | 0 | N | orconazole |
| B1-1932 | EtO | O | O | CO₂H | — | — | 0 | N | orconazole |
| B1-1933 | t-BuO | O | O | CO₂H | — | — | 0 | N | orconazole |
| B1-1934 | cyclohexylO | O | O | CO₂H | — | — | 0 | N | orconazole |
| B1-1935 | iPrO | O | O | CO₂H | — | — | 0 | N | orconazole |
| B1-1936 | PhO | O | O | CO₂H | — | — | 0 | N | orconazole |
| B1-1937 | (HOCH₂— | O | O | CO₂H | — | — | 0 | N | orconazole |
| | CH₂)₂N | | | | | | | | |
| B1-1938 | N(H)NMe₂ | O | O | CO₂H | — | — | 0 | N | orconazole |
| B1-1939 | 4-(OH)Ph | O | O | CO₂H | — | — | 0 | N | orconazole |
| B1-1940 | 4-(NH₂)Ph | O | O | CO₂H | — | — | 0 | N | orconazole |
| B1-1941 | EtS | O | O | CO₂H | — | — | 0 | N | oxiconazole |
| B1-1942 | EtO | O | O | CO₂H | — | — | 0 | N | oxiconazole |
| B1-1943 | tBuO | O | 0 | CO₂H | — | — | 0 | N | oxiconazole |
| B1-1944 | cyclohexylO | O | O | CO₂H | — | — | 0 | N | oxiconazole |
| B1-1945 | iPrO | O | O | CO₂H | — | — | 0 | N | oxiconazole |
| B1-1946 | PhO | O | O | CO₂H | — | — | 0 | N | oxiconazole |
| B1-1947 | (HOCH₂— | O | O | CO₂H | — | — | 0 | N | oxiconazole |
| | CH₂)₂N | | | | | | | | |
| B1-1948 | N(H)NMe₂ | O | O | CO₂H | — | — | 0 | N | oxiconazole |
| B1-1949 | 4-(OH)Ph | O | O | CO₂H | — | — | 0 | N | oxiconazole |
| B1-1950 | 4-(NH₂)Ph | O | O | CO₂H | — | — | 0 | N | oxiconazole |
| B1-1951 | EtS | O | O | CO₂H | — | — | 0 | N | parconazole |
| B1-1952 | EtO | O | O | CO₂H | — | — | 0 | N | parconazole |
| B1-1953 | t-BuO | O | O | CO₂H | — | — | 0 | N | parconazole |
| B1-1954 | cyclohexylO | O | O | CO₂H | — | — | 0 | N | parconazole |
| B1-1955 | iPrO | O | O | CO₂H | — | — | 0 | N | parconazole |
| B1-1956 | PhO | O | 0 | CO₂H | — | — | 0 | N | parconazole |
| B1-1957 | (HOCH₂— | O | O | CO₂H | — | — | 0 | N | parconazole |
| | CH₂)₂N | | | | | | | | |
| B1-1958 | N(H)NMe₂ | O | O | CO₂H | — | — | 0 | N | parconazole |
| B1-1959 | 4-(OH)Ph | O | O | CO₂H | — | — | 0 | N | parconazole |
| B1-1960 | 4-(NH₂)Ph | O | O | CO₂H | — | — | 0 | N | parconazole |
| B1-1961 | EtS | O | O | CO₂H | — | — | 0 | N | ravuconazole |
| B1-1962 | EtO | O | O | CO₂H | — | — | 0 | N | ravuconazole |
| B1-1963 | t-BuO | O | O | CO₂H | — | — | 0 | N | ravuconazole |
| B1-1964 | cyclohexylO | O | O | CO₂H | — | — | 0 | N | ravuconazole |
| B1-1965 | iPrO | O | O | CO₂H | — | — | 0 | N | ravuconazole |
| B1-1966 | PhO | O | O | CO₂H | — | — | 0 | N | ravuconazole |
| B1-1967 | (HOCH₂— | O | O | CO₂H | — | — | 0 | N | ravuconazole |
| | CH₂)₂N | | | | | | | | |
| B1-1968 | N(H)NMe₂ | O | O | CO₂H | — | — | 0 | N | ravuconazole |
| B1-1969 | 4-(OH)Ph | O | O | CO₂H | — | — | 0 | N | ravuconazole |
| B1-1970 | 4-(NH₂)Ph | O | O | CO₂H | — | — | 0 | N | ravuconazole |
| B1-1971 | EtS | O | O | CO₂H | — | — | 0 | N | sertaconazole |
| B1-1972 | EtO | O | O | CO₂H | — | — | 0 | N | sertaconazole |
| B1-1973 | t-BuO | O | O | CO₂H | — | — | 0 | N | sertaconazole |
| B1-1974 | cyclohexylO | O | O | CO₂H | — | — | 0 | N | sertaconazole |
| B1-1975 | iPrO | O | O | CO₂H | — | — | 0 | N | sertaconazole |
| B1-1976 | PhO | O | O | CO₂H | — | — | 0 | N | sertaconazole |
| B1-1977 | (HOCH₂— | O | O | CO₂H | — | — | 0 | N | sertaconazole |
| | CH₂)₂N | | | | | | | | |
| B1-1978 | N(H)NMe₂ | O | O | CO₂H | — | — | 0 | N | sertaconazole |
| B1-1979 | 4-(OH)Ph | O | O | CO₂H | — | — | 0 | N | sertaconazole |
| B1-1980 | 4-(NH₂)Ph | O | O | CO₂H | — | — | 0 | N | sertaconazole |
| B1-1981 | EtS | O | O | CO₂H | — | — | 0 | N | sulconazole |
| B1-1982 | EtO | O | O | CO₂H | — | — | 0 | N | sulconazole |
| B1-1983 | t-BuO | O | O | CO₂H | — | — | 0 | N | sulconazole |
| B1-1984 | cyclohexylO | O | O | CO₂H | — | — | 0 | N | sulconazole |
| B1-1985 | iPrO | O | O | CO₂H | — | — | 0 | N | sulconazole |
| B1-1986 | PhO | O | O | CO₂H | — | — | 0 | N | sulconazole |
| B1-1987 | (HOCH₂— | O | O | CO₂H | — | — | 0 | N | sulconazole |
| | CH₂)₂N | | | | | | | | |
| B1-1988 | N(H)NMe₂ | O | O | CO₂H | — | — | 0 | N | sulconazole |
| B1-1989 | 4-(OH)Ph | O | O | CO₂H | — | — | 0 | N | sulconazole |
| B1-1990 | 4-(NH₂)Ph | O | O | CO₂H | — | — | 0 | N | sulconazole |
| B1-1991 | EtS | O | O | CO₂H | — | — | 0 | N | tioconazole |
| B1-1992 | EtO | O | O | CO₂H | — | — | 0 | N | tioconazole |
| B1-1993 | t-BuO | O | O | CO₂H | — | — | 0 | N | tioconazole |
| B1-1994 | cyclohexylO | O | O | CO₂H | — | — | 0 | N | tioconazole |
| B1-1995 | iPrO | O | O | CO₂H | — | — | 0 | N | tioconazole |
| B1-1996 | Ph0 | O | O | CO₂H | — | — | 0 | N | tioconazole |
| B1-1997 | (HOCH₂— | O | O | CO₂H | — | — | 0 | N | tioconazole |
| | CH₂)₂N | | | | | | | | |
| B1-1998 | N(H)NMe₂ | O | O | CO₂H | — | — | 0 | N | tioconazole |
| B1-1999 | 4-(OH)Ph | O | O | CO₂H | — | — | 0 | N | tioconazole |
| B1-2000 | 4-(NH₂)Ph | O | O | CO₂H | — | — | 0 | N | tioconazole |
| B1-2001 | EtS | O | O | CO₂H | — | — | 0 | N | valconazole |
| B1-2002 | EtO | O | O | CO₂H | — | — | 0 | N | valconazole |
| B1-2003 | t-BuO | O | O | CO₂H | — | — | 0 | N | valconazole |
| B1-2004 | cyclohexylO | O | O | CO₂H | — | — | 0 | N | valconazole |
| B1-2005 | iPrO | O | O | CO₂H | — | — | 0 | N | valconazole |
| B1-2006 | PhO | O | O | CO₂H | — | — | 0 | N | valconazole |
| B1-2007 | (HOCH₂— | O | O | CO₂H | — | — | 0 | N | valconazole |
| | CH₂)₂N | | | | | | | | |
| B1-2008 | N(H)NMe₂ | O | O | CO₂H | — | — | 0 | N | valconazole |
| B1-2009 | 4-(OH)Ph | O | O | CO₂H | — | — | 0 | N | valconazole |
| B1-2010 | 4-(NH₂)Ph | O | O | CO₂H | — | — | 0 | N | valconazole |
| B1-2011 | EtS | O | O | CO₂H | — | — | 0 | N | zinoconazole |
| B1-2012 | EtO | O | O | CO₂H | — | — | 0 | N | zinoconazole |
| B1-2013 | t-BuO | O | O | CO₂H | — | — | 0 | N | zinoconazole |
| B1-2014 | cyclohexylO | O | O | CO₂H | — | — | 0 | N | zinoconazole |
| B1-2015 | iPrO | O | O | CO₂H | — | — | 0 | N | zinoconazole |
| B1-2016 | PhO | O | O | CO₂H | — | — | 0 | N | zinoconazole |
| B1-2017 | (HOCH₂— | O | O | CO₂H | — | — | 0 | N | zinoconazole |
| | CH₂)₂N | | | | | | | | |
| B1-2018 | N(H)NMe₂ | O | O | CO₂H | — | — | 0 | N | zinoconazole |
| B1-2019 | 4-(OH)Ph | O | O | CO₂H | — | — | 0 | N | zinoconazole |
| B1-2020 | 4-(NH₂)Ph | O | O | CO₂H | — | — | 0 | N | zinoconazole |
| B1-2021 | EtS | O | O | CO₂H | O | O | 1 | C | cloxacillin |
| B1-2022 | EtO | O | O | CO₂H | O | O | 1 | C | cloxacillin |
| B1-2023 | t-BuO | O | O | CO₂H | O | O | 1 | C | cloxacillin |
| B1-2024 | cyclohexylO | O | O | CO₂H | O | O | 1 | C | cloxacillin |
| B1-2025 | iPrO | O | O | CO₂H | O | O | 1 | C | cloxacillin |
| B1-2026 | PhO | O | O | CO₂H | O | O | 1 | C | cloxacillin |
| B1-2027 | (HOCH₂— | O | O | CO₂H | O | O | 1 | C | cloxacillin |
| | CH₂)₂N | | | | | | | | |
| B1-2028 | N(H)NMe₂ | O | O | CO₂H | O | O | 1 | C | cloxacillin |
| B1-2029 | 4-(OH)Ph | O | O | CO₂H | O | O | 1 | C | cloxacillin |
| B1-2030 | 4-(NH₂)Ph | O | O | CO₂H | O | O | 1 | C | cloxacillin |
| B1-2031 | EtS | O | O | CO₂H | O | O | 1 | C | valproic acid |
| B1-2032 | EtO | O | O | CO₂H | O | O | 1 | C | valproic acid |
| B1-2033 | t-BuO | O | O | CO₂H | O | O | 1 | C | valproic acid |
| B1-2034 | cyclohexylO | O | O | CO₂H | O | O | 1 | C | valproic acid |
| B1-2035 | iPrO | O | O | CO₂H | O | O | 1 | C | valproic acid |
| B1-2036 | PhO | O | O | CO₂H | O | O | 1 | C | valproic acid |
| B1-2037 | (HOCH₂— | O | O | CO₂H | O | O | 1 | C | valproic acid |
| | CH₂)₂N | | | | | | | | |
| B1-2038 | N(H)NMe₂ | O | O | CO₂H | O | O | 1 | C | valproic acid |
| B1-2039 | 4-(OH)Ph | O | O | CO₂H | O | O | 1 | C | valproic acid |
| B1-2040 | 4-(NH₂)Ph | O | O | CO₂H | O | O | 1 | C | valproic acid |
| B1-2041 | EtS | O | O | CO₂H | O | O | 1 | C | retinoic acid |
| B1-2042 | EtO | O | O | CO₂H | O | O | 1 | C | retinoic acid |
| B1-2043 | t-BuO | O | O | CO₂H | O | O | 1 | C | retinoic acid |
| B1-2044 | cyclohexylO | O | O | CO₂H | O | O | 1 | C | retinoic acid |
| B1-2045 | iPrO | O | O | CO₂H | O | O | 1 | C | retinoic acid |
| B1-2046 | PhO | O | O | CO₂H | O | O | 1 | C | retinoic acid |
| B1-2047 | (HOCH₂— | O | O | CO₂H | O | O | 1 | C | retinoic acid |
| | CH₂)₂N | | | | | | | | |
| B1-2048 | N(H)NMe₂ | O | O | CO₂H | O | O | 1 | C | retinoic acid |
| B1-2049 | 4-(OH)Ph | O | O | CO₂H | O | O | 1 | C | retinoic acid |
| B1-2050 | 4-(NH₂)Ph | O | O | CO₂H | O | O | 1 | C | retinoic acid |
| B1-2051 | EtS | O | O | CO₂H | O | O | 1 | C | oxaprozin |
| B1-2052 | EtO | O | O | CO₂H | O | O | 1 | C | oxaprozin |
| B1-2053 | t-BuO | O | O | CO₂H | O | O | 1 | C | oxaprozin |
| B1-2054 | cyclohexylO | O | O | CO₂H | O | O | 1 | C | oxaprozin |
| B1-2055 | iPrO | O | O | CO₂H | O | O | 1 | C | oxaprozin |
| B1-2056 | PhO | O | O | CO₂H | O | O | 1 | C | oxaprozin |
| B1-2057 | (HOCH₂— | O | O | CO₂H | O | O | 1 | C | oxaprozin |
| | CH₂)₂N | | | | | | | | |
| B1-2058 | N(H)NMe₂ | O | O | CO₂H | O | O | 1 | C | oxaprozin |
| B1-2059 | 4-(OH)Ph | O | O | CO₂H | O | O | 1 | C | oxaprozin |
| B1-2060 | 4-(NH₂)Ph | O | O | CO₂H | O | O | 1 | C | oxaprozin |
| B1-2061 | EtS | O | O | CO₂H | O | O | 1 | C | naproxen |
| B1-2062 | EtO | O | O | CO₂H | O | O | 1 | C | naproxen |
| B1-2063 | t-BuO | O | O | CO₂H | O | O | 1 | C | naproxen |
| B1-2064 | cyclohexylO | O | O | CO₂H | O | O | 1 | C | naproxen |
| B1-2065 | iPrO | O | O | CO₂H | O | O | 1 | C | naproxen |
| B1-2066 | PhO | O | O | CO₂H | O | O | 1 | C | naproxen |
| B1-2067 | (HOCH₂— | O | O | CO₂H | O | O | 1 | C | naproxen |
| | CH₂)₂N | | | | | | | | |
| B1-2068 | N(H)NMe₂ | O | O | CO₂H | O | O | 1 | C | naproxen |
| B1-2069 | 4-(OH)Ph | O | O | CO₂H | O | O | 1 | C | naproxen |
| B1-2070 | 4-(NH₂)Ph | O | O | CO₂H | O | O | 1 | C | naproxen |
| B1-2071 | EtS | O | O | CO₂H | O | O | 1 | C | monopril |
| B1-2072 | EtO | O | O | CO₂H | O | O | 1 | C | monopril |
| B1-2073 | t-BuO | O | O | CO₂H | O | O | 1 | C | monopril |
| B1-2074 | cyclohexylO | O | O | CO₂H | O | O | 1 | C | monopril |
| B1-2075 | iPrO | O | O | CO₂H | O | O | 1 | C | monopril |
| B1-2076 | PhO | O | O | CO₂H | O | O | 1 | C | monopril |
| B1-2077 | (HOCH₂— | O | O | CO₂H | O | O | 1 | C | monopril |
| | CH₂)₂N | | | | | | | | |
| B1-2078 | N(H)NMe₂ | O | O | CO₂H | O | O | 1 | C | monopril |
| B1-2079 | 4-(OH)Ph | O | O | CO₂H | O | O | 1 | C | monopril |
| B1-2080 | 4-(NH₂)Ph | O | O | CO₂H | O | O | 1 | C | monopril |
| B1-2081 | EtS | O | O | CO₂H | O | O | 1 | C | ketorolac |
| B1-2082 | EtO | O | O | CO₂H | O | O | 1 | C | ketorolac |
| B1-2083 | t-BuO | O | O | CO₂H | O | O | 1 | C | ketorolac |
| B1-2084 | cyclohexylO | O | O | CO₂H | O | O | 1 | C | ketorolac |
| B1-2085 | iPrO | O | O | CO₂H | O | O | 1 | C | ketorolac |
| B1-2086 | PhO | O | O | CO₂H | O | O | 1 | C | ketorolac |
| B1-2087 | (HOCH₂— | O | O | CO₂H | O | O | 1 | C | ketorolac |
| | CH₂)₂N | | | | | | | | |
| B1-2088 | N(H)NMe₂ | O | O | CO₂H | O | O | 1 | C | ketorolac |
| B1-2089 | 4-(OH)Ph | O | O | CO₂H | O | O | 1 | C | ketorolac |
| B1-2090 | 4-(NH₂)Ph | O | O | CO₂H | O | O | 1 | C | ketorolac |
| B1-2091 | EtS | O | O | CO₂H | O | O | 1 | C | ketoprofen |
| B1-2092 | EtO | O | O | CO₂H | O | O | 1 | C | ketoprofen |
| B1-2093 | t-BuO | O | O | CO₂H | O | O | 1 | C | ketoprofen |
| B1-2094 | cyclohexylO | O | O | CO₂H | O | O | 1 | C | ketoprofen |
| B1-2095 | iPrO | O | O | CO₂H | O | O | 1 | C | ketoprofen |
| B1-2096 | Ph0 | O | O | CO₂H | O | O | 1 | C | ketoprofen |
| B1-2097 | (HOCH₂— | O | O | CO₂H | O | O | 1 | C | ketoprofen |
| | CH₂)₂N | | | | | | | | |
| B1-2098 | N(H)NMe₂ | O | O | CO₂H | O | O | 1 | C | ketoprofen |
| B1-2099 | 4-(OH)Ph | O | O | CO₂H | O | O | 1 | C | ketoprofen |
| B1-2100 | 4-(NH₂)Ph | O | O | CO₂H | O | O | 1 | C | ketoprofen |
| B1-2101 | EtS | O | O | CO₂H | O | O | 1 | C | indomethacin |
| B1-2102 | EtO | O | O | CO₂H | O | O | 1 | C | indomethacin |
| B1-2103 | t-BuO | O | O | CO₂H | O | O | 1 | C | indomethacin |
| B1-2104 | cyclohexylO | O | O | CO₂H | O | O | 1 | C | indomethacin |
| B1-2105 | iPrO | O | O | CO₂H | O | O | 1 | C | indomethacin |
| B1-2106 | PhO | O | O | CO₂H | O | O | 1 | C | indomethacin |
| B1-2107 | (HOCH₂— | O | O | CO₂H | O | O | 1 | C | indomethacin |
| | CH₂)₂N | | | | | | | | |
| B1-2108 | N(H)NMe₂ | O | O | CO₂H | O | O | 1 | C | indomethacin |
| B1-2109 | 4-(OH)Ph | O | O | CO₂H | O | O | 1 | C | indomethacin |
| B1-2110 | 4-(NH₂)Ph | O | O | CO₂H | O | O | 1 | C | indomethacin |
| B1-2111 | EtS | O | O | CO₂H | O | O | 1 | C | ibuprofen |
| B1-2112 | EtO | O | O | CO₂H | O | O | 1 | C | ibuprofen |
| B1-2113 | i-BuO | O | O | CO₂H | O | O | 1 | C | ibuprofen |
| B1-2114 | cyclohexylO | O | O | CO₂H | O | O | 1 | C | ibuprofen |
| B1-2115 | iPrO | O | O | CO₂H | O | O | 1 | C | ibuprofen |
| B1-2116 | PhO | O | O | CO₂H | O | O | 1 | C | ibuprofen |
| B1-2117 | (HOCH₂— | O | O | CO₂H | O | O | 1 | C | ibuprofen |
| | CH₂)₂N | | | | | | | | |
| B1-2118 | N(H)NMe₂ | O | O | CO₂H | O | O | 1 | C | ibuprofen |
| B1-2119 | 4-(OH)Ph | O | O | CO₂H | O | O | 1 | C | ibuprofen |
| B1-2120 | 4-(NH₂)Ph | O | O | CO₂H | O | O | 1 | C | ibuprofen |
| B1-2121 | EtS | O | O | CO₂H | O | O | 1 | C | gemfibrozil |
| B1-2122 | EtO | O | O | CO₂H | O | O | 1 | C | gemfibrozil |
| B1-2123 | t-BuO | O | O | CO₂H | O | O | 1 | C | gemfibrozil |
| B1-2124 | cyclohexylO | O | O | CO₂H | O | O | 1 | C | gemfibrozil |
| B1-2125 | iPrO | O | O | CO₂H | O | O | 1 | C | gemfibrozil |
| B1-2126 | PhO | O | O | CO₂H | O | O | 1 | C | gemfibrozil |
| B1-2127 | (HOCH₂— | O | O | CO₂H | O | O | 1 | C | gemfibrozil |
| | CH₂)₂N | | | | | | | | |
| B1-2128 | N(H)NMe₂ | O | O | CO₂H | O | O | 1 | C | gemfibrozil |
| B1-2129 | 4-(OH)Ph | O | O | CO₂H | O | O | 1 | C | gemfibrozil |
| B1-2130 | 4-(NH₂)Ph | O | O | CO₂H | O | O | 1 | C | gemfibrozil |
| B1-2131 | EtS | O | O | CO₂H | O | O | 1 | C | flurbiprofen |
| B1-2132 | EtO | O | O | CO₂H | O | O | 1 | C | flurbiprofen |
| B1-2133 | t-BuO | O | O | CO₂H | O | O | 1 | C | flurbiprofen |
| B1-2134 | cyclohexylO | O | O | CO₂H | O | O | 1 | C | flurbiprofen |
| B1-2135 | iPrO | O | O | CO₂H | O | O | 1 | C | flurbiprofen |
| B1-2136 | PhO | O | O | CO₂H | O | O | 1 | C | flurbiprofen |
| B1-2137 | (HOCH₂— | O | O | CO₂H | O | O | 1 | C | flurbiprofen |
| | CH₂)₂N | | | | | | | | |
| B1-2138 | N(H)NMe₂ | O | O | CO₂H | O | O | 1 | C | flurbiprofen |
| B1-2139 | 4-(OH)Ph | O | O | CO₂H | O | O | 1 | C | flurbiprofen |
| B1-2140 | 4-(NH₂)Ph | O | O | CO₂H | O | O | 1 | C | flurbiprofen |
| B1-2141 | EtS | O | O | CO₂H | O | O | 1 | C | arthrocine |
| B1-2142 | EtO | O | O | CO₂H | O | O | 1 | C | arthrocine |
| B1-2143 | t-BuO | O | O | CO₂H | O | O | 1 | C | arthrocine |
| B1-2144 | cyclohexylO | O | O | CO₂H | O | O | 1 | C | arthrocine |
| B1-2145 | iPrO | O | O | CO₂H | O | O | 1 | C | arthrocine |
| B1-2146 | PhO | O | O | CO₂H | O | O | 1 | C | arthrocine |
| B1-2147 | (HOCH₂— | O | O | CO₂H | O | O | 1 | C | arthrocine |
| | CH₂)₂N | | | | | | | | |
| B1-2148 | N(H)NMe₂ | O | O | CO₂H | O | O | 1 | C | arthrocine |
| B1-2149 | 4-(OH)Ph | O | O | CO₂H | O | O | 1 | C | arthrocine |
| B1-2150 | 4-(NH₂)Ph | O | O | CO₂H | O | O | 1 | C | arthrocine |
| B1-2151 | EtS | O | O | CO₂H | O | O | 1 | C | adapalene |
| B1-2152 | EtO | O | O | CO₂H | O | O | 1 | C | adapalene |
| B1-2153 | t-BuO | O | O | CO₂H | O | O | 1 | C | adapalene |
| B1-2154 | cyclohexylO | O | O | CO₂H | O | O | 1 | C | adapalene |
| B1-2155 | iPrO | O | O | CO₂H | O | O | 1 | C | adapalene |
| B1-2156 | PhO | O | O | CO₂H | O | O | 1 | C | adapalene |
| B1-2157 | (HOCH₂— | O | O | CO₂H | O | O | 1 | C | adapalene |
| | CH₂)₂N | | | | | | | | |
| B1-2158 | N(H)NMe₂ | O | O | CO₂H | O | O | 1 | C | adapalene |
| B1-2159 | 4-(OH)Ph | O | O | CO₂H | O | O | 1 | C | adapalene |
| B1-2160 | 4-(NH₂)Ph | O | O | CO₂H | O | O | 1 | C | adapalene |
| B1-2161 | EtS | O | O | CO₂H | O | O | 1 | C | lansoprazole |
| B1-2162 | EtO | O | O | CO₂H | O | O | 1 | C | lansoprazole |
| B1-2163 | t-BuO | O | O | CO₂H | O | O | 1 | C | lansoprazole |
| B1-2164 | cyclohexylO | O | O | CO₂H | O | O | 1 | C | lansoprazole |
| B1-2165 | iPrO | O | O | CO₂H | O | O | 1 | C | lansoprazole |
| B1-2166 | PhO | O | O | CO₂H | O | O | 1 | C | lansoprazole |
| B1-2167 | (HOCH₂— | O | O | CO₂H | O | O | 1 | C | lansoprazole |
| | CH₂)₂N | | | | | | | | |
| B1-2168 | N(H)NMe₂ | O | O | CO₂H | O | O | 1 | C | lansoprazole |
| B1-2169 | 4-(OH)Ph | O | O | CO₂H | O | O | 1 | C | lansoprazole |
| B1-2170 | 4-(NH₂)Ph | O | O | CO₂H | O | O | 1 | C | lansoprazole |
| B1-2171 | EtS | O | O | CO₂H | O | O | 1 | C | lovastatin |
| B1-2172 | EtO | O | O | CO₂H | O | O | 1 | C | lovastatin |
| B1-2173 | t-Bu0 | O | O | CO₂H | O | O | 1 | C | lovastatin |
| B1-2174 | cyclohexylO | O | O | CO₂H | O | O | 1 | C | lovastatin |
| B1-2175 | iPrO | O | O | CO₂H | O | O | 1 | C | lovastatin |
| B1-2176 | PhO | O | O | CO₂H | O | O | 1 | C | lovastatin |
| B1-2177 | (HOCH₂— | O | O | CO₂H | O | O | 1 | C | lovastatin |
| | CH₂)₂N | | | | | | | | |
| B1-2178 | N(H)NMe₂ | O | O | CO₂H | O | O | 1 | C | lovastatin |
| B1-2179 | 4-(OH)Ph | O | O | CO₂H | O | O | 1 | C | lovastatin |
| B1-2180 | 4-(NH₂)Ph | O | O | CO₂H | O | O | 1 | C | lovastatin |
| B1-2181 | EtS | O | O | CO₂H | O | O | 1 | C | warfarin |
| B1-2182 | EtO | O | O | CO₂H | O | O | 1 | C | warfarin |
| B1-2183 | t-Bu0 | O | O | CO₂H | O | O | 1 | C | warfarin |
| B1-2184 | cyclohexylO | O | O | CO₂H | O | O | 1 | C | warfarin |
| B1-2185 | iPrO | O | O | CO₂H | O | O | 1 | C | warfarin |
| B1-2186 | PhO | O | O | CO₂H | O | O | 1 | C | warfarin |
| B1-2187 | (HOCH₂— | O | O | CO₂H | O | O | 1 | C | warfarin |
| | CH₂)₂N | | | | | | | | |
| B1-2188 | N(H)NMe₂ | O | O | CO₂H | O | O | 1 | C | warfarin |
| B1-2189 | 4-(OH)Ph | O | O | CO₂H | O | O | 1 | C | warfarin |
| B1-2190 | 4-(NH₂)Ph | O | O | CO₂H | O | O | 1 | C | warfarin |
| B1-2191 | EtS | O | O | CO₂H | — | — | 0 | N | tolteridine |
| B1-2192 | EtO | O | O | CO₂H | — | — | 0 | N | tolteridine |
| B1-2193 | t-BuO | O | O | CO₂H | — | — | 0 | N | tolteridine |
| B1-2194 | cyclohexylO | O | O | CO₂H | — | — | 0 | N | tolteridine |
| B1-2195 | iPrO | O | O | CO₂H | — | — | 0 | N | tolteridine |
| B1-2196 | PhO | O | O | CO₂H | — | — | 0 | N | tolteridine |
| B1-2197 | (HOCH₂— | O | O | CO₂H | — | — | 0 | N | tolteridine |
| | CH₂)₂N | | | | | | | | |
| B1-2198 | N(H)NMe₂ | O | O | CO₂H | — | — | 0 | N | tolteridine |
| B1-2199 | 4-(OH)Ph | O | O | CO₂H | — | — | 0 | N | tolteridine |
| B1-2200 | 4-(NH₂)Ph | O | O | CO₂H | — | — | 0 | N | tolteridine |
| B1-2201 | EtS | O | O | CO₂H | — | — | 0 | N | ticlopidine |
| B1-2202 | EtO | O | O | CO₂H | — | — | 0 | N | ticlopidine |
| B1-2203 | t-BuO | O | O | CO₂H | — | — | 0 | N | ticlopidine |
| B1-2204 | cyclohexylO | O | O | CO₂H | — | — | 0 | N | ticlopidine |
| B1-2205 | iPrO | O | O | CO₂H | — | — | 0 | N | ticlopidine |
| B1-2206 | PhO | O | O | CO₂H | — | — | 0 | N | ticlopidine |
| B1-2207 | (HOCH₂— | O | O | CO₂H | — | — | 0 | N | ticlopidine |
| | CH₂)₂N | | | | | | | | |
| B1-2208 | N(H)NMe₂ | O | O | CO₂H | — | — | 0 | N | ticlopidine |
| B1-2209 | 4-(OH)Ph | O | O | CO₂H | — | — | 0 | N | ticlopidine |
| B1-2210 | 4-(NH₂)Ph | O | O | CO₂H | — | — | 0 | N | ticlopidine |
| B1-2211 | EtS | O | O | CO₂H | — | — | 0 | N | sibutramine |
| B1-2212 | EtO | O | O | CO₂H | — | — | 0 | N | sibutramine |
| B1-2213 | t-BuO | O | O | CO₂H | — | — | 0 | N | sibutramine |
| B1-2214 | cyclohexylO | O | O | CO₂H | — | — | 0 | N | sibutramine |
| B1-2215 | iPrO | O | O | CO₂H | — | — | 0 | N | sibutramine |
| B1-2216 | PhO | O | O | CO₂H | — | — | 0 | N | sibutramine |
| B1-2217 | (HOCH₂— | O | O | CO₂H | — | — | 0 | N | sibutramine |
| | CH₂)₂N | | | | | | | | |
| B1-2218 | N(H)NMe₂ | O | O | CO₂H | — | — | 0 | N | sibutramine |
| B1-2219 | 4-(OH)Ph | O | O | CO₂H | — | — | 0 | N | sibutramine |
| B1-2220 | 4-(NH₂)Ph | O | O | CO₂H | — | — | 0 | N | sibutramine |
| B1-2221 | EtS | O | O | CO₂H | — | — | 0 | N | propoxyphene |
| B1-2222 | EtO | O | O | CO₂H | — | — | 0 | N | propoxyphene |
| B1-2223 | t-BuO | O | O | CO₂H | — | — | 0 | N | propoxyphene |
| B1-2224 | cyclohexylO | O | O | CO₂H | — | — | 0 | N | propoxyphene |
| B1-2225 | iPrO | O | O | CO₂H | — | — | 0 | N | propoxyphene |
| B1-2226 | PhO | O | O | CO₂H | — | — | 0 | N | propoxyphene |
| B1-2227 | (HOCH₂— | O | O | CO₂H | — | — | 0 | N | propoxyphene |
| | CH₂)₂N | | | | | | | | |
| B1-2228 | N(H)NMe₂ | O | O | —CO₂H | — | — | 0 | N | propoxyphene |
| B1-2229 | 4-(OH)Ph | O | O | CO₂H | — | — | 0 | N | propoxyphene |
| B1-2230 | 4-(NH₂)Ph | O | O | CO₂H | — | — | 0 | N | propoxyphene |
| B1-2231 | EtS | O | O | CO₂H | — | — | 0 | N | nifurantin |
| B1-2232 | EtO | O | 0 | CO₂H | — | — | 0 | N | nifurantin |
| B1-2233 | t-BuO | O | O | CO₂H | — | — | 0 | N | nifurantin |
| B1-2234 | cyclohexylO | O | O | CO₂H | — | — | 0 | N | nifurantin |
| B1-2235 | iPrO | O | O | CO₂H | — | — | 0 | N | nifurantin |
| B1-2236 | PhO | O | O | CO₂H | — | — | 0 | N | nifurantin |
| B1-2237 | (HOCH₂— | O | O | CO₂H | — | — | 0 | N | nifurantin |
| | CH₂)₂N | | | | | | | | |
| B1-2238 | N(H)NMe₂ | O | O | CO₂H | — | — | 0 | N | nifurantin |
| B1-2239 | 4-(OH)Ph | O | O | CO₂H | — | — | 0 | N | nifurantin |
| B1-2240 | 4-(NH₂)Ph | O | O | CO₂H | — | — | 0 | N | nifurantin |
| B1-2241 | EtS | O | O | CO₂H | — | — | 0 | N | nefazodone |
| B1-2242 | EtO | O | O | CO₂H | — | — | 0 | N | nefazodone |
| B1-2243 | t-BuO | O | O | CO₂H | — | — | 0 | N | nefazodone |
| B1-2244 | cyclohexylO | O | O | CO₂H | — | — | 0 | N | nefazodone |
| B1-2245 | iPrO | O | O | CO₂H | — | — | 0 | N | nefazodone |
| B1-2246 | PhO | O | O | CO₂H | — | — | 0 | N | nefazodone |
| B1-2247 | (HOCH₂— | O | O | CO₂H | — | — | 0 | N | nefazodone |
| | CH₂)₂N | | | | | | | | |
| B1-2248 | N(H)NMe₂ | O | O | CO₂H | — | — | 0 | N | nefazodone |
| B1-2249 | 4-(OH)Ph | O | O | CO₂H | — | — | 0 | N | nefazodone |
| B1-2250 | 4-(NH₂)Ph | O | O | CO₂H | — | — | 0 | N | nefazodone |
| B1-2251 | EtS | O | O | CO₂H | — | — | 0 | N | donezapil |
| B1-2252 | EtO | O | O | CO₂H | — | — | 0 | N | donezapil |
| B1-2253 | t-BuO | O | O | CO₂H | — | — | 0 | N | donezapil |
| B1-2254 | cyclohexylO | O | O | CO₂H | — | — | 0 | N | donezapil |
| B1-2255 | iPrO | O | O | CO₂H | — | — | 0 | N | donezapil |
| B1-2256 | PhO | O | O | CO₂H | — | — | 0 | N | donezapil |
| B1-2257 | (HOCH₂— | O | O | CO₂H | — | — | 0 | N | donezapil |
| | CH₂)₂N | | | | | | | | |
| B1-2258 | N(H)NMe₂ | O | O | CO₂H | — | — | 0 | N | donezapil |
| B1-2259 | 4-(OH)Ph | O | O | CO₂H | — | — | 0 | N | donezapil |
| B1-2260 | 4-(NH₂)Ph | O | O | CO₂H | — | — | 0 | N | donezapil |
| B1-2261 | EtS | O | O | CO₂H | — | — | 0 | N | dicodid |
| B1-2262 | EtO | O | O | CO₂H | — | — | 0 | N | dicodid |
| B1-2263 | t-BuO | O | O | CO₂H | — | — | 0 | N | dicodid |
| B1-2264 | cyclohexylO | O | O | CO₂H | — | — | 0 | N | dicodid |
| B1-2265 | iPrO | O | O | CO₂H | — | — | 0 | N | dicodid |
| B1-2266 | PhO | O | O | CO₂H | — | — | 0 | N | dicodid |
| B1-2267 | (HOCH₂— | O | O | CO₂H | — | — | 0 | N | dicodid |
| | CH₂)₂N | | | | | | | | |
| B1-2268 | N(H)NMe₂ | O | O | CO₂H | — | — | 0 | N | dicodid |
| B1-2269 | 4-(OH)Ph | O | O | CO₂H | — | — | 0 | N | dicodid |
| B1-2270 | 4-(NH₂)Ph | O | O | CO₂H | — | — | 0 | N | dicodid |
| B1-2271 | EtS | O | O | CO₂H | — | — | 0 | N | coichicine |
| B1-2272 | EtO | O | O | CO₂H | — | — | 0 | N | coichicine |
| B1-2273 | t-BuO | O | O | CO₂H | — | — | 0 | N | coichicine |
| B1-2274 | cyclohexylO | O | O | CO₂H | — | — | 0 | N | coichicine |
| B1-2275 | iPrO | O | O | CO₂H | — | — | 0 | N | coichicine |
| B1-2276 | Ph0 | O | O | CO₂H | — | — | 0 | N | coichicine |
| B1-2277 | (HOCH₂— | O | O | CO₂H | — | — | 0 | N | coichicine |
| | CH₂)₂N | | | | | | | | |
| B1-2278 | N(H)NMe₂ | O | O | CO₂H | — | — | 0 | N | coichicine |
| B1-2279 | 4-(OH)Ph | O | O | CO₂H | — | — | 0 | N | coichicine |
| B1-2280 | 4-(NH₂)Ph | O | O | CO₂H | — | — | 0 | N | coichicine |
| B1-2281 | EtS | O | O | CO₂H | — | — | 0 | N | citalopram |
| B1-2282 | EtO | O | O | CO₂H | — | — | 0 | N | citalopram |
| B1-2283 | t-BuO | O | O | CO₂H | — | — | 0 | N | citalopram |
| B1-2284 | cyclohexylO | O | O | CO₂H | — | — | 0 | N | citalopram |
| B1-2285 | iPrO | O | O | GCO₂H | — | — | 0 | N | citalopram |
| B1-2286 | Ph0 | O | O | CO₂H | — | — | 0 | N | citalopram |
| B1-2287 | (HOCH₂— | O | O | CO₂H | — | — | 0 | N | citalopram |
| | CH₂)₂N | | | | | | | | |
| B1-2288 | N(H)NMe₂ | O | O | CO₂H | — | — | 0 | N | citalopram |
| B1-2289 | 4-(OH)Ph | O | O | CO₂H | — | — | 0 | N | citalopram |
| B1-2290 | 4-(NH₂)Ph | O | O | CO₂H | — | — | 0 | N | citalopram |
| B1-2291 | EtS | O | O | CO₂H | — | — | 0 | N | benzatropine |
| B1-2292 | EtO | O | O | CO₂H | — | — | 0 | N | benzatropine |
| B1-2293 | t-BuO | O | O | CO₂H | — | — | 0 | N | benzatropine |
| B1-2294 | cyclohexylO | O | O | CO₂H | — | — | 0 | N | benzatropine |
| B1-2295 | iPrO | O | O | CO₂H | — | — | 0 | N | benzatropine |
| B1-2296 | PhO | O | O | CO₂H | — | — | 0 | N | benzatropine |
| B1-2297 | (HOCH₂— | O | O | CO₂H | — | — | 0 | N | benzatropine |
| | CH₂)₂N | | | | | | | | |
| B1-2298 | N(H)NMe₂ | O | O | CO₂H | — | — | 0 | N | benzatropine |
| B1-2299 | 4-(OH)Ph | O | O | CO₂H | — | — | 0 | N | benzatropine |
| B1-2300 | 4-(NH₂)Ph | O | O | CO₂H | — | — | 0 | N | benzatropine |

Table B2: Compounds (B2-1)-(B2-2300) are compounds of Formula I where X², Z¹, G¹⁰, G¹¹, R², G²⁰, G²¹′ m, t, q and Z²(X²)—[(C=G²⁰)-G²¹]-H, Z²(X²)_{q}—H or Z²(X²) are identical to those in Table B1 except for R¹ which equals Co₂Me.

Table B3: Compounds (B3-1)-(B3-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂Et.

Table B4: Compounds (B4-1)-(B4-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B 1 except for R¹ which equals CO₂₁t-Pr. Table B5: Compounds (B5-1)-(B5-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂t-Bu.

Table B6: Compounds (B6-1)-(B6-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals Co₂cyclopropyl.

Table B7: Compounds (B7-1)-(B7-2300) are compounds of Formula I where X², Z¹¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals Co₂Bn.

Table B8: Compounds (B8-1)-(B8-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals Co₂CH₂CH₂Ph.

Table B9: Compounds (B9-1)-(B9-2300) are compounds of Formula I where X², Z¹, G¹⁰, G¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂CH2CF₃.

Table B10: Compounds (B10-1)-(B10-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals Co₂CH₂OMe.

Table B11: Compounds (B11-1)-(B11-2300) are compounds of Formula I where X², Z¹, G¹⁰, G¹⁰, R², G²⁰, G²¹, m, t, q and Z²(X²)—[(C=G²⁰)-G²¹]-H, Z²(X²)_{q}—H or Z²) are identical to those in Table B1 except for R¹ which equals Co₂allyl.

Table B12: Compounds (B12-1)-(B12-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂Ph.

Table B13: Compounds (B13-1)-(B13-2300) are compounds of Formula I where X², Z¹, G¹⁰, G¹¹, R², G²0, G²¹, m, t, q and Z²(X²)—[(C=G²⁰)-G²¹]-H, Z²(X²)_{q}—H or Z²(X²) are identical to those in Table B1 except for R¹ which equals CO₂-2(OMe)Ph.

Table B14: Compounds (B14-1)-(B14-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂-3-(OMe)Ph.

Table B15: Compounds (B15-1)-(B15-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂-4-(OMe)-Ph.

Table B16: Compounds (B16-1)-(B16-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals Co₂-2(Me)Ph.

Table B17: Compounds (B17-1)-(B17-2300) are compounds of Formula I where X², Z¹, G¹⁰, G¹¹, R², G²⁰, G², m, t, q and Z²(X²)—[(C=G²⁰)-G²¹]-H, Z²(X²)_{q}—H or Z²(X²) are identical to those in Table B1 except for R¹ which equals Co₂-3(Me)Ph.

Table-B18: Compounds (B18-1)-(B18-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂-4(Me)Ph.

Table B19: Compounds (B19-1)-(B19-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂-4(NO₂)Ph.

Table B20: Compounds (B20-1)-(B20-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂SiMe₃.

Table B21: Compounds (B21-1)-(B21-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(H)Me.

Table B22: Compounds B22-1)-(B22-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(—O)NMe₂.

Table B23: Compounds (B23-1)-(B23-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)NBn₂. Table B24: Compounds (B24-1)-(B24-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(Me)Bn.

Table B25: Compounds (B25-1)-(B25-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(Me)Ph.

Table B26: Compounds B26-1)-(B26-2300) are compounds of Formula I where X², Z¹, G¹⁰, G¹¹, R², G²⁰, G²¹, m, t, q and Z²(X²)—[(C=G²0)-G²¹]-H, Z²(2)_{q}—H or Z²(X²) are identical to those in Table B1 except for R¹ which equals C(═O)N(E)Ph.

Table B27: Compounds (B27-1)-(B27-2300) are compounds of Formula I where X², Z¹, G¹⁰, G¹¹, R², G²⁰, G²¹, m, t, q and Z²(X²)—[(C=G²⁰)G²¹]-H, Z²(X²)_{q}—H or Z²(X¹) are identical to those in Table B1 except for R¹ which equals C(═O)N(H)NMe₂.

Table B28: Compounds (B28-1)-(B28-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals CO₂-octyl.

Table B29: Compounds (B29-1)-(B29-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(H)ₙ-Bu.

Table B30: Compounds (B30-1)-(B30-2300) are compounds of Formula I where X², Z¹¹, G¹⁰, G¹¹, R², G²⁰, G²¹, m, t, q and Z²(X²)—[(C=G²⁰)-G²¹]-H, Z²(X²)_{q}—H or Z²(X²), are identical to those in Table B1 except for R¹ which equals C(═O)N(H)N(H)Co₂Et.

Table B31: Compounds (B31-1)-(B31-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B13 except for R¹ which equals C(═O)N(H)N(H)Co₂t-Bu.

Table B32: Compounds (B32-1)-(B32-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B13 except for R¹ which equals C(═O)N(H)N═CHMe.

Table B33: Compounds (B33-1)-(B33-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(H)N═CMe₂.

Table B34: Compounds (B34-1)-(B34-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(H)N═CHPh.

Table B35: Compounds (B35-1)-(B3-52300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)SPh.

Table B36: Compounds (B36-1)-(B36-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)SEt.

Table B37: Compounds (B37-1)-(B37-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)Sn-Bu.

Table B38: Compounds (B38-1)-(B38-2300) are compounds of Formula I where X², Z¹, G¹⁰, G¹¹, R², G²⁰, G²¹, m, t, q and Z²(X²)—[(C=G²⁰)-G²]-H, Z²(X²)_{q}H or Z²(X²) are identical to those in Table B1 except for R¹ which equals C(═O)N(H)OMe.

Table B39: Compounds (B39-1)-(B39-2300) are compounds of Formula I where X², Z¹, G¹⁰, G¹¹, R², G²⁰; G²¹, m, t, q and Z²(X²)—[(C=G²⁰)-G²¹]-H, Z²(X²)_{q}—H or Z²(X²) are identical to those in Table B1 except for R¹ which equals C(═O)N(H)OBn.

Table B40: Compounds (B40-1)-(B40-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(H)NH₂.

Table B41: Compounds (B41-1)-(B41-2300) are compounds of Formula I where X², Z¹, G¹⁰, G¹¹, R², G²⁰, G²¹, m, t, q and Z²(X²)—[(C=G²⁰)-G²¹]-H, Z²(X²)_{q}—H or Z²(X²) are identical to those in Table B1 except for R¹ which equals C(═O)N(H)OH.

Table C describes additional examples of compounds of Formula I which can be made using the procedures described hereinbefore, where R² is hydrogen, m=1, q=0, t=0 or 1 and the pharmaceutical (a veterinary drug) which defines the pharmaceutical moiety of these examples is Z¹(X¹)ₘ—H. The following groups, Z¹(X¹)ₘ—H, X¹, G¹⁰, G¹¹, R¹, G²⁰, G²¹, t and Z² are defined within Table C.

TABLE C

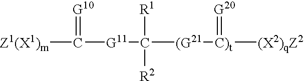

| Cmpd # | Z¹(X¹)ₘ-H | X¹ | G¹⁰ | G¹¹ | R¹ | G²⁰ | G²¹ | t | Z² |
|---|---|---|---|---|---|---|---|---|---|
| C-1 | albendazole | N | O | O | CO₂H | O | O | 1 | methyl |
| C-2 | clioquinol | O | O | O | CO₂H | O | O | 1 | C(CH₃)₃ |
| C-3 | febantel | N | O | O | CO₂H | O | O | 1 | Ph |
| C-4 | fenbendazole | N | O | O | CO₂H | O | O | 1 | 4Me(Ph) |
| C-5 | guaifenesin | O | O | O | CO₂H | — | — | 0 | Cl |
| C-6 | mibolerone | O | O | O | CO₂H | — | — | 0 | {N(Et)₃}⁺Cl⁻ |
| C-7 | omeprazole | N | O | O | CO₂H | — | — | 0 | {N(Me)₂Ph}⁺Cl⁻ |
| C-8 | oxazepam | O | O | O | CO₂Me | O | O | 1 | methyl |
| C-9 | piroxicam | N | O | O | CO₂Me | O | O | 1 | C(CH₃)₃ |
| C-10 | primidone | N | O | O | CO₂Me | O | O | 1 | Ph |
| C-11 | procainamide | N | O | O | CO₂Me | O | O | 1 | 4Me(Ph) |
| C-12 | thiabendazole | N | O | O | CO₂Me | — | — | 0 | Cl |
| C-13 | warfarin | O | O | O | CO₂Me | — | — | 0 | {N(Et)₃}⁺Cl⁻ |
| C-14 | albendazole | N | O | O | CO₂Me | — | — | 0 | {N(Me)₂Ph}⁺Cl⁻ |
| C-15 | clioquinol | O | O | O | CO₂Et | O | O | 1 | methyl |
| C-16 | febantel | N | O | O | CO₂Et | — | — | 0 | Cl |
| C-17 | fenbendazole | N | O | S | CO₂n-Pr | O | O | 1 | methyl |
| C-18 | guaifenesin | O | O | S | CO₂n-Pr | — | — | 0 | Cl |
| C-19 | mibolerone | O | O | O | CO₂n-Bu | O | O | 1 | methyl |
| C-20 | omeprazole | N | O | O | CO₂n-Bu | O | O | 1 | C(CH₃)₃ |
| C-21 | oxazepam | O | O | O | CO₂n-Bu | O | O | 1 | Ph |
| C-22 | piroxicam | N | O | S | CO₂n-Bu | O | O | 1 | 4Me(Ph) |
| C-23 | primidone | N | O | S | CO₂n-Bu | O | O | 1 | CF₃CH₂ |
| C-24 | procainamide | N | O | O | CO₂n-Bu | — | — | 0 | Cl |
| C-25 | thiabendazole | N | O | O | CO₂n-Bu | — | — | 0 | {N(Et)₃}⁺Cl⁻ |
| C-26 | warfarin | O | O | O | CO₂n-Bu | — | — | 0 | {N(Me)₂Ph}⁺Cl⁻ |
| C-27 | albendazole | N | O | O | CO₂n-Bu | — | — | 0 | O—C(CH₃)₃ |
| C-28 | clioquinol | O | O | O | CO₂—Bn | O | O | 1 | methyl |
| C-29 | febantel | N | O | O | CO₂—Bn | O | O | 1 | C(CH₃)₃ |
| C-30 | fenbendazole | N | O | O | CO₂—Bn | O | O | 1 | Ph |
| C-31 | guaifenesin | O | O | S | CO₂—Bn | O | O | 1 | 4Me(Ph) |
| C-32 | mibolerone | O | O | S | CO₂—Bn | O | O | 1 | CF₃CH₂ |
| C-33 | omeprazole | N | O | O | CO₂—Bn | — | — | 0 | Cl |
| C-34 | oxazepam | O | O | O | CO₂—Bn | — | — | 0 | {N(Et)₃}⁺Cl⁻ |
| C-35 | piroxicam | N | O | O | CO₂—Bn | — | — | 0 | {N(Me)₂Ph}⁺Cl⁻ |
| C-36 | primidone | N | O | O | CO₂—Bn | O | O | 0 | O—C(CH₃)₃ |
| C-37 | procainamide | N | O | O | COSMe | O | O | 1 | methyl |
| C-38 | thiabendazole | N | O | S | COSMe | O | O | 1 | C(CH₃)₃ |
| C-39 | warfarin | O | O | O | COSMe | — | — | 0 | Cl |
| C-40 | albendazole | N | O | O | COSMe | O | O | 0 | {N(Et)₃}⁺Cl⁻ |
| C-41 | clioquinol | O | O | S | COSEt | O | O | 1 | methyl |
| C-42 | febantel | N | O | O | COSEt | — | — | 0 | Cl |
| C-43 | fenbendazole | N | O | O | COSn-Bu | O | O | 1 | methyl |
| C-44 | guaifenesin | O | O | S | COSn-Bu | — | — | 0 | Cl |
| C-45 | mibolerone | O | O | O | COSBn | O | O | 1 | methyl |
| C-46 | omeprazole | N | O | O | COSBn | — | — | 0 | Cl |
| C-47 | oxazepam | O | O | O | CONHMe | O | O | 1 | methyl |
| C-48 | piroxicam | N | O | O | CONHMe | O | O | 1 | C(CH₃)₃ |
| C-49 | primidone | N | O | O | CONHEt | O | O | 1 | methyl |
| C-50 | procainamide | N | O | O | CONHn-Pr | O | O | 1 | methyl |
| C-51 | thiabendazole | N | O | O | CONHn-Bu | O | O | 1 | methyl |
| C-52 | warfarin | O | O | O | CONHn-Bu | O | O | 1 | C(CH₃)₃ |
| C-53 | albendazole | N | O | O | CONHn-Bu | O | O | 1 | Ph |
| C-54 | clioquinol | O | O | O | CONHn-Bu | O | O | 1 | 4Me(Ph) |
| C-55 | febantel | N | O | O | CONHn-Bu | O | O | 1 | CF₃CH₂ |
| C-56 | fenbendazole | N | O | O | CONHn-Bu | — | — | 0 | Cl |
| C-57 | guaifenesin | O | O | O | CONHn-Bu | O | O | O | {N(Et)₃}⁺Cl⁻ |
| C-58 | mibolerone | O | O | O | CONH—Bn | O | O | 1 | methyl |
| C-59 | omeprazole | N | O | O | CONH—Bn | O | O | 1 | C(CH₃)₃ |
| C-60 | oxazepam | O | O | O | CONH—Bn | — | — | 0 | {N(Et)₃}⁺Cl⁻ |
| C-61 | piroxicam | N | O | O | CONNH₂ | O | O | 1 | methyl |
| C-62 | primidone | N | O | O | CONHNHMe | O | O | 1 | methyl |
| C-63 | procainamide | N | O | O | CONHNHEt | O | O | 1 | methyl |
| C-64 | thiabendazole | N | O | O | CONHNHPr | O | O | 1 | methyl |
| C-65 | warfarin | O | O | O | CONHNHt-Bu | O | O | 1 | C(CH₃)₃ |
| C-66 | albendazole | N | O | O | CONHNHCO₂Me | O | O | 1 | C(CH₃)₃ |
| C-67 | clioquinol | O | O | O | CONHNHCO₂Et | O | O | 1 | C(CH₃)₃ |
| C-68 | febantel | N | O | O | CONHNHCO₂t-Bu | O | O | 1 | C(CH₃)₃ |
| C-69 | fenbendazole | N | O | O | CONHNHCO₂Bn | O | O | 1 | C(CH₃)₃ |
| C-70 | guaifenesin | O | O | O | CONHN═CHMe | O | O | 1 | methyl |
| C-71 | mibolerone | O | O | O | CONHN═CHMe | — | — | 0 | {N(Et)₃}⁺Cl⁻ |
| C-72 | omeprazole | N | O | O | CONHN═CHPh | O | O | 1 | methyl |
| C-73 | oxazepam | O | O | O | CONHN═CHPh | O | O | 1 | C(CH₃)₃ |
| C-74 | disopyramide | N | O | O | CO₂H | O | O | 1 | methyl |
| C-75 | disopyramide | N | O | O | CO₂H | O | O | 1 | C(CH₃)₃ |
| C-76 | disopyramide | N | O | O | CO₂H | O | O | 1 | Ph |
| C-77 | disopyramide | N | O | O | CO₂H | O | O | 1 | 4Me(Ph) |
| C-78 | disopyramide | N | O | O | CO₂H | — | — | 0 | Cl |
| C-79 | disopyramide | N | O | O | CO₂H | — | — | 0 | {N(Et)₃}⁺Cl⁻ |
| C-80 | disopyramide | N | O | O | CO₂H | — | — | 0 | {N(Me)₂Ph}⁺Cl⁻ |
| C-81 | disopyramide | N | O | O | CO₂Me | O | O | 1 | methyl |
| C-82 | disopyramide | N | O | O | CO₂Me | O | O | 1 | C(CH₃)₃ |
| C-83 | disopyramide | N | O | O | CO₂Me | O | O | 1 | Ph |
| C-84 | disopyramide | N | O | O | CO₂Me | O | O | 1 | 4Me(Ph) |
| C-85 | disopyramide | N | O | O | CO₂Me | — | — | 0 | Cl |
| C-86 | aminocaproic acid | N | O | O | CO₂Me | — | — | 0 | {N(Et)₃}⁺Cl⁻ |
| C-87 | aminocaproic acid | N | O | O | CO₂Me | — | — | 0 | {N(Me)₂Ph}⁺Cl⁻ |
| C-88 | aminocaproic acid | N | O | O | CO₂Et | O | O | 1 | methyl |
| C-89 | aminocaproic acid | N | O | O | CO₂Et | — | — | 0 | Cl |
| C-90 | aminophylline | N | O | S | CO₂n-Pr | O | O | 1 | methyl |
| C-91 | aminophylline | N | O | S | CO₂n-Pr | — | — | 0 | Cl |
| C-92 | aminophylline | N | O | O | CO₂n-Bu | O | O | 1 | methyl |
| C-93 | aminophylline | N | O | O | CO₂n-Bu | O | O | 1 | C(CH₃)₃ |
| C-94 | aminophylline | N | O | O | CO₂n-Bu | O | O | 1 | Ph |
| C-95 | aminophylline | N | O | S | CO₂n-Bu | O | O | 1 | 4Me(Ph) |
| C-96 | aminophylline | N | O | S | CO₂n-Bu | O | O | 1 | CF₃CH₂ |
| C-97 | aminophylline | N | O | O | CO₂n-Bu | — | — | 0 | Cl |
| C-98 | aminophylline | N | O | O | CO₂n-Bu | — | — | 0 | {N(Et)₃}⁺Cl⁻ |
| C-99 | aminophylline | N | O | O | CO₂n-Bu | — | — | 0 | {N(Me)₂Ph}⁺Cl⁻ |
| C-100 | aminophylline | N | O | O | CO₂n-Bu | — | — | 0 | O—C(CH₃)₃ |
| C-101 | amprolium | N | O | O | CO₂-Bn | O | O | 1 | methyl |
| C-102 | amprolium | N | O | O | CO₂-Bn | O | O | 1 | O(CH₃)₃ |
| C-103 | amprolium | N | O | O | CO₂-Bn | O | O | 1 | Ph |
| C-104 | amprolium | N | O | S | CO₂-Bn | O | O | 1 | 4Me(Ph) |
| C-105 | amprolium | N | O | S | CO₂-Bn | O | O | 1 | CF₃CH₂ |
| C-106 | atipamezole | N | O | O | CO₂-Bn | — | — | 0 | Cl |
| C-107 | atipamezole | N | O | O | CO₂-Bn | — | — | 0 | {N(Et)₃}⁺Cl⁻ |
| C-108 | atipamezole | N | O | O | CO₂-Bn | — | — | 0 | {N(Me)₂Ph}⁺Cl⁻ |
| C-109 | atipamezole | N | O | O | CO₂-Bn | — | — | 0 | OC(CH₃)₃ |
| C-110 | atipamezole | N | O | O | COSMe | O | O | 1 | methyl |
| C-111 | atipamezole | N | O | S | COSMe | O | O | 1 | C(CH₃)₃ |
| C-112 | atipamezole | N | O | O | COSMe | — | — | 0 | Cl |
| C-113 | atipamezole | N | O | O | COSMe | — | — | 0 | {N(Et)₃}⁺Cl⁻ |
| C-114 | benazepril | N | O | S | COSEt | O | O | 1 | methyl |
| C-115 | benazepril | N | O | O | COSEt | — | — | 0 | Cl |
| C-116 | benazepril | N | O | O | COSn-Bu | O | O | 1 | methyl |
| C-117 | benazepril | N | O | S | COSn-Bu | — | — | 0 | Cl |
| C-118 | benazepril | N | O | O | COSBn | O | O | 1 | methyl |
| C-119 | benazepril | N | O | O | COSBn | — | — | 0 | Cl |
| C-120 | benazepril | N | O | O | CONHMe | O | O | 1 | methyl |
| C-121 | benazepril | N | O | O | CONHMe | O | O | 1 | C(CH₃)₃ |
| C-122 | benazepril | N | O | O | CONHEt | O | O | 1 | methyl |
| C-123 | cisapride | N | O | O | CONHn-Pr | O | O | 1 | methyl |
| C-124 | detomidine | N | O | O | CONHn-Bu | O | O | 1 | methyl |
| C-125 | enalapril | O | O | O | CONHn-Bu | O | O | 1 | C(CH₃)₃ |
| C-126 | enalapril | O | O | O | CONHn-Bu | O | O | 1 | Ph |
| C-127 | enalapril | O | O | O | CONHn-Bu | O | O | 1 | 4Me(Ph) |
| C-128 | enalapril | O | O | O | CONHn-Bu | O | O | 1 | CF₃CH₂ |
| C-129 | enalapril | O | O | O | CONHn-Bu | — | — | 0 | Cl |
| C-130 | enalapril | O | O | O | CONHn-Bu | — | — | 0 | {N(Et)}⁺Cl⁻ |
| C-131 | enalapril | N | O | O | CONH—Bn | O | O | 1 | methyl |
| C-132 | enalapril | N | O | O | CONH—Bn | O | O | 1 | C(CH₃)₃ |
| C-133 | enalapril | N | O | O | CONH—Bn | — | — | 0 | {N(Et)₃}⁺Cl⁻ |
| C-134 | enalapril | N | O | O | CONNH₂ | O | O | 1 | methyl |
| C-135 | enalapril | N | O | O | CONHNHMe | O | O | 1 | methyl |
| C-136 | enalapril | N | O | O | CONHNHEt | O | O | 1 | methyl |
| C-137 | enalapril | N | O | O | CONHNHPr | O | O | 1 | methyl |
| C-138 | enalapril | N | O | O | CONHNHt-Bu | O | O | 1 | C(CH₃)₃ |
| C-139 | enalapril | N | O | O | CONHNHCO₂Me | O | O | 1 | C(CH₃)₃ |
| C-140 | enalapril | N | O | O | CONHNHCO₂Et | O | O | 1 | C(CH₃)₃ |
| C-141 | enalapril | N | O | O | CONHNHCO₂t-Bu | O | O | 1 | C(CH₃)₃ |
| C-142 | enalapril | N | O | O | CONHNHCO₂Bn | O | O | 1 | C(CH₃)₃ |
| C-143 | enalapril | N | O | O | CONHN═CHMe | O | O | 1 | methyl |
| C-144 | enalapril | N | O | O | CONHN═CHMe | — | — | 0 | {N(Et)₃}⁺Cl⁻ |
| C-145 | enalapril | N | O | O | CONHN═CHPh | O | O | 1 | methyl |
| C-146 | enalapril | N | O | O | CONHN═CHPh | O | O | 1 | C(CH₃)₃ |
| C-147 | enalapril | N | O | O | CO₂H | O | O | 1 | methyl |
| C-148 | enalapril | N | O | O | CO₂H | O | O | 1 | C(CH₃)₃ |
| C-149 | enalapril | N | O | O | CO₂H | O | O | 1 | Ph |
| C-150 | enalapril | N | O | O | CO₂H | O | O | 1 | 4Me(Ph) |
| C-151 | enalapril | N | O | O | CO₂H | — | — | 0 | Cl |
| C-152 | enalapril | N | O | O | CO₂H | — | — | 0 | {N(Et)₃}⁺Cl⁻ |
| C-153 | enalapril | N | O | O | CO₂H | — | — | 0 | {N(Me)₂Ph}⁺Cl⁻ |
| C-154 | enalapril | N | O | O | CO₂Me | O | O | 1 | methyl |
| C-155 | enalapril | N | O | O | CO₂Me | O | O | 1 | C(CH₃)₃ |
| C-156 | enalapril | N | O | O | CO₂Me | O | O | 1 | Ph |
| C-157 | fluconazole | N | O | O | CO₂H | O | O | 1 | methyl |
| C-158 | fluconazole | N | O | O | CO₂H | O | O | 1 | C(CH₃)₃ |
| C-159 | fluconazole | N | O | O | CO₂Me | O | O | 1 | Ph |
| C-160 | fluconazole | N | O | O | CO₂Me | O | O | 1 | 4Me(Ph) |
| C-161 | fluconazole | N | O | O | CO₂Me | — | — | 0 | Cl |
| C-162 | fluconazole | N | O | O | CO₂Me | — | — | 0 | {N(Et)₃}⁺Cl⁻ |
| C-163 | fluconazole | N | O | O | CO₂Me | — | — | 0 | {N(Me)₂Ph}⁺Cl⁻ |
| C-164 | fluconazole | N | O | O | CO₂Me | O | O | 1 | methyl |
| C-165 | fluconazole | N | O | O | CO₂Me | O | O | 1 | C(CH₃)₃ |
| C-166 | fluconazole | N | O | O | CO₂Et | O | O | 1 | Ph |
| C-167 | fluconazole | N | O | O | CO₂n-Pr | O | O | 1 | 4Me(Ph) |
| C-168 | fluconazole | N | O | O | CO₂n-Bu | — | — | 0 | Cl |
| C-169 | fluconazole | N | O | O | CO₂—Bn | — | — | 0 | {N(Et)₃}⁺Cl⁻ |
| C-170 | fluconazole | N | O | O | COSMe | — | — | 0 | {N(Me)₂Ph}⁺Cl⁻ |
| C-171 | fluconazole | N | O | O | COSEt | O | O | 1 | methyl |
| C-172 | fluconazole | N | O | O | COSn-Bu | O | O | 1 | C(CH₃)₃ |
| C-173 | fluconazole | N | O | O | CONHMe | O | O | 1 | Ph |
| C-174 | fluconazole | N | O | O | CONHEt | O | O | 1 | 4Me(Ph) |
| C-175 | fluconazole | N | O | O | CONHn-Bu | — | — | 0 | Cl |
| C-176 | fluconazole | N | O | O | CONH—Bn | — | — | 0 | {N(Et)₃}⁺Cl⁻ |
| C-177 | fluconazole | N | O | O | CONNH₂ | — | — | 0 | {N(Me)₂Ph}⁺Cl⁻ |
| C-178 | fluconazole | N | O | O | CONHNHMe | O | O | 1 | methyl |
| C-179 | fluconazole | N | O | O | CONHNHEt | O | O | 1 | C(CH₃)₃ |
| C-180 | fluconazole | N | O | O | CONHNHPr | O | O | 1 | Ph |
| C-181 | fluconazole | N | O | O | CONHNHt-Bu | O | O | 1 | 4Me(Ph) |
| C-182 | fluconazole | N | O | O | CONHNHCO₂Me | — | — | 0 | Cl |
| C-183 | fluconazole | N | O | O | CONHNHCO₂Et | — | — | 0 | {N(Et)₃}⁺Cl⁻ |
| C-184 | fluconazole | N | O | O | CONHNHCO₂t-Bu | — | — | 0 | {N(Me)₂Ph}⁺Cl⁻ |
| C-185 | fluconazole | N | O | O | CONHNHCO₂Bn | O | O | 1 | methyl |
| C-186 | fluconazole | N | O | O | CONHN═CHMe | O | O | 1 | C(CH₃)₃ |
| C-187 | fluconazole | N | O | O | CONHN═CHMe | O | O | 1 | 4Me(Ph) |
| C-188 | fluconazole | N | O | O | CONHN═CHPh | — | — | 0 | Cl |
| C-189 | fluconazole | N | O | O | CONHN═CHPh | — | — | 0 | {N(Et)₃}⁺Cl⁻ |
| C-190 | hetacillin | N | O | O | CO₂Me | O | O | 1 | 4Me(Ph) |
| C-191 | imidacloprid | N | O | O | CO₂Me | — | — | 0 | Cl |
| C-192 | imidacloprid | N | O | O | CO₂Me | — | — | 0 | {N(Et)₃}⁺Cl⁻ |
| C-193 | imidacloprid | N | O | O | CO₂Me | — | — | 0 | {N(Me)₂Ph}⁺Cl⁻ |
| C-194 | imidacloprid | N | O | O | CO₂Et | O | O | 1 | methyl |
| C-195 | imidacloprid | N | O | O | CO₂Et | — | — | 0 | Cl |
| C-196 | imidacloprid | N | O | S | CO₂n-Pr | O | O | 1 | methyl |
| C-197 | imidacloprid | N | O | S | CO₂n-Pr | — | — | 0 | Cl |
| C-198 | imidacloprid | N | O | O | CO₂n-Bu | O | O | 1 | methyl |
| C-199 | imidacloprid | N | O | O | CO₂n-Bu | O | O | 1 | C(CH₃)₃ |
| C-200 | imidacloprid | N | O | O | CO₂n-Bu | O | O | 1 | Ph |
| C-201 | imidacloprid | N | O | S | CO₂n-Bu | O | O | 1 | 4Me(Ph) |
| C-202 | imidacloprid | N | O | S | CO₂n-Bu | O | O | 1 | CF₃CH₂ |
| C-203 | imidacloprid | N | O | O | CO₂n-Bu | — | — | 0 | Cl |
| C-204 | imidacloprid | N | O | O | CO₂n-Bu | — | — | 0 | {N(Et)₃}⁺Cl⁻ |
| C-205 | imidacloprid | N | O | O | CO₂n-Bu | — | — | 0 | {N(Me)₂Ph}⁺Cl⁻ |
| C-206 | imidacloprid | N | O | O | CO₂n-Bu | — | — | 0 | OC(CH₃)₃ |
| C-207 | imidacloprid | N | O | O | CO₂—Bn | O | O | 1 | methyl |
| C-208 | imidacloprid | N | O | O | CO₂—Bn | O | O | 1 | C(CH₃)₃ |
| C-209 | imidacloprid | N | O | O | CO₂—Bn | O | O | 1 | Ph |
| C-210 | imidacloprid | N | O | S | CO₂—Bn | O | O | 1 | 4Me(Ph) |
| C-211 | imidacloprid | N | O | S | CO₂—Bn | O | O | 1 | CF₃CH₂ |
| C-212 | imidacloprid | N | O | O | CO₂—Bn | — | — | 0 | Cl |
| C-213 | imidacloprid | N | O | O | CO₂—Bn | — | — | 0 | {N(Et)₃}⁺Cl⁻ |
| C-214 | imidacloprid | N | O | O | CO₂—Bn | — | — | 0 | {N(Me)₂Ph}⁺Cl⁻ |
| C-215 | imidacloprid | N | O | O | CO₂—Bn | — | — | 0 | OC(CH₃)₃ |
| C-216 | imidacloprid | N | O | O | COSMe | O | O | 1 | methyl |
| C-217 | imidacloprid | N | O | S | COSMe | O | O | 1 | C(CH₃)₃ |
| C-218 | imidacloprid | N | O | O | COSMe | — | — | 0 | Cl |
| C-219 | imidacloprid | N | O | O | COSMe | — | — | 0 | {N(Et)₃}⁺Cl⁻ |
| C-220 | imidacloprid | N | O | S | COSEt | O | O | 1 | methyl |
| C-221 | imidacloprid | N | O | O | COSEt | — | — | 0 | Cl |
| C-222 | imidacloprid | N | O | O | COSn-Bu | O | O | 1 | methyl |
| C-223 | imidacloprid | N | O | S | COSn-Bu | — | — | 0 | Cl |
| C-224 | imidacloprid | N | O | O | COSBn | O | O | 1 | methyl |
| C-225 | imidacloprid | N | O | O | COSBn | — | — | 0 | Cl |
| C-226 | imidacloprid | N | O | O | CONHMe | O | O | 1 | methyl |
| C-227 | imidacloprid | N | O | O | CONHMe | O | O | 1 | C(CH₃)₃ |
| C-228 | imidacloprid | N | O | O | CONHEt | O | O | 1 | methyl |
| C-229 | imidacloprid | N | O | O | CONHn-Pr | O | O | 1 | methyl |
| C-230 | imidacloprid | N | O | O | CONHn-Bu | O | O | 1 | methyl |
| C-231 | imidacloprid | N | O | O | CONHn-Bu | O | O | 1 | C(CH₃)₃ |
| C-232 | imidacloprid | N | O | O | CONHn-Bu | O | O | 1 | Ph |
| C-233 | imidacloprid | N | O | O | CONHn-Bu | O | O | 1 | 4Me(Ph) |
| C-234 | imidacloprid | N | O | O | CONHn-Bu | O | O | 1 | CF₃CH₂ |
| C-235 | imidacloprid | N | O | O | CONHn-Bu | — | — | 0 | Cl |
| C-236 | imidacloprid | N | O | O | CONHn-Bu | — | — | 0 | {N(Et)₃}⁺Cl⁻ |
| C-237 | imidacloprid | N | O | O | CONH—Bn | O | O | 1 | methyl |
| C-238 | imidacloprid | N | O | O | CONH—Bn | O | O | 1 | C(CH₃)₃ |
| C-239 | imidacloprid | N | O | O | CONH—Bn | — | — | 0 | {N(Et)₃}⁺Cl⁻ |
| C-240 | imidacloprid | N | O | O | CONNH₂ | O | O | 1 | methyl |
| C-241 | ketamine | N | O | O | CONHNHMe | O | O | 1 | methyl |
| C-242 | ketamine | N | O | O | CONHNHEt | O | O | 1 | methyl |
| C-243 | ketamine | N | O | O | CONHNHPr | O | O | 1 | methyl |
| C-244 | ketamine | N | O | O | CONHNHt-Bu | O | O | 1 | C(CH₃)₃ |
| C-245 | ketamine | N | O | O | CONHNHCO₂Me | O | O | 1 | C(CH₃)₃ |
| C-246 | ketamine | N | O | O | CONHNHCO₂Et | O | O | 1 | C(CH₃)₃ |
| C-247 | ketamine | N | O | O | CONHNHCO₂t-Bu | O | O | 1 | C(CH₃)₃ |
| C-248 | ketamine | N | O | O | CONHNHCO₂Bn | O | O | 1 | C(CH₃)₃ |
| C-249 | ketamine | N | O | O | CONHN═CHMe | O | O | 1 | methyl |
| C-250 | ketamine | N | O | O | CONHN═CHMe | — | — | 0 | {N(Et)₃}⁺Cl⁻ |
| C-251 | ketamine | N | O | O | CONHN═CHPh | O | O | 1 | methyl |
| C-252 | ketamine | N | O | O | CONHN═CHPh | O | O | 1 | C(CH₃)₃ |
| C-253 | lidocaine | N | O | O | CO₂H | O | O | 1 | methyl |
| C-254 | lincomycin | N | O | O | CO₂H | O | O | 1 | C(CH₃)₃ |
| C-255 | lomustine | N | O | O | CO₂H | O | O | 1 | Ph |
| C-256 | lomustine | N | O | O | CO₂H | O | O | 1 | 4Me(Ph) |
| C-257 | lomustine | N | O | O | CO₂H | — | — | 0 | Cl |
| C-258 | lomustine | N | O | O | CO₂H | — | — | 0 | {N(Et)₃}⁺Cl⁻ |
| C-259 | lomustine | N | O | O | CO₂H | — | — | 0 | {N(Me)₂Ph}⁺Cl⁻ |
| C-260 | lomustine | N | O | O | CO₂Me | O | O | 1 | methyl |
| C-261 | lomustine | N | O | O | CO₂Me | O | O | 1 | C(CH₃)₃ |
| C-262 | lomustine | N | O | O | CO₂Me | O | O | 1 | Ph |
| C-263 | lomustine | N | O | O | CO₂Me | O | O | 1 | 4Me(Ph) |
| C-264 | lomustine | N | O | O | CO₂Me | — | — | 0 | Cl |
| C-265 | lomustine | N | O | O | CO₂Me | — | — | 0 | {N(Et)₃}⁺Cl⁻ |
| C-266 | lomustine | N | O | O | CO₂Me | — | — | 0 | {N(Me)₂Ph}⁺Cl⁻ |
| C-267 | lomustine | N | O | O | CO₂Et | O | O | 1 | methyl |
| C-268 | lomustine | N | O | O | CO₂Et | — | — | 0 | Cl |
| C-269 | lomustine | N | O | S | CO₂n-Pr | O | O | 1 | methyl |
| C-270 | lomustine | N | O | S | CO₂n-Pr | — | — | 0 | Cl |
| C-271 | lomustine | N | O | O | CO₂n-Bu | O | O | 1 | methyl |
| C-272 | lomustine | N | O | O | CO₂n-Bu | O | O | 1 | C(CH₃)₃ |
| C-273 | mechlorethamine | N | O | O | CO₂n-Bu | O | O | 1 | Ph |
| C-274 | mechlorethamine | N | O | S | CO₂n-Bu | O | O | 1 | 4Me(Ph) |
| C-275 | mechlorethamine | N | O | S | CO₂n-Bu | O | O | 1 | CF₃CH₂ |
| C-276 | mechlorethamine | N | O | O | CO₂n-Bu | — | — | 0 | Cl |
| C-277 | mechlorethamine | N | O | O | CO₂n-Bu | — | — | 0 | {N(Et)₃}⁺Cl⁻ |
| C-278 | mechlorethamine | N | O | O | CO₂n-Bu | — | — | 0 | {N(Me)₂Ph}⁺Cl⁻ |
| C-279 | mechlorethamine | N | O | O | CO₂n-Bu | — | — | 0 | O—C(CH₃)₃ |
| C-280 | meclofenamic acid | N | O | O | CO₂—Bn | O | O | 1 | methyl |
| C-281 | mercaptopurine | N | O | O | CO₂—Bn | O | O | 1 | C(CH₃)₃ |
| C-282 | methotrexate | N | O | O | CO₂—Bn | O | O | 1 | Ph |
| C-283 | mexiletine | N | O | S | CO₂—Bn | O | O | 1 | 4Me(Ph) |
| C-284 | mexiletine | N | O | S | CO₂—Bn | O | O | 1 | CF₃CH₂ |
| C-285 | mexiletine | N | O | O | CO₂—Bn | — | — | 0 | Cl |
| C-286 | mexiletine | N | O | O | CO₂—Bn | — | — | 0 | {N(Et)₃}⁺Cl⁻ |
| C-287 | mexiletine | N | O | O | CO₂—Bn | — | — | 0 | {N(Me)₂Ph}⁺Cl⁻ |
| C-288 | mexiletine | N | O | O | CO₂—Bn | — | — | 0 | OC(CH₃)₃ |
| C-289 | mexiletine | N | O | O | COSMe | O | O | 1 | methyl |
| C-290 | mexiletine | N | O | S | COSMe | O | O | 1 | C(CH₃)₃ |
| C-291 | mexiletine | N | O | O | COSMe | — | — | 0 | Cl |
| C-292 | mexiletine | N | O | O | COSMe | — | — | 0 | {N(Et)₃}⁺Cl⁻ |
| C-293 | mexiletine | N | O | S | COSEt | O | O | 1 | methyl |
| C-294 | mexiletine | N | O | O | COSEt | — | — | 0 | Cl |
| C-295 | mexiletine | N | O | O | COSn-Bu | O | O | 1 | methyl |
| C-296 | mexiletine | N | O | S | COSn-Bu | — | — | 0 | Cl |
| C-297 | mexiletine | N | O | O | COSBn | O | O | 1 | methyl |
| C-298 | mexiletine | N | O | O | COSBn | — | — | 0 | Cl |
| C-299 | mexiletine | N | O | O | CONHMe | O | O | 1 | methyl |
| C-300 | mexiletine | N | O | O | CONHMe | O | O | 1 | C(CH₃)₃ |
| C-301 | mexiletine | N | O | O | CONHEt | O | O | 1 | methyl |
| C-302 | mexiletine | N | O | O | CONHn-Pr | O | O | 1 | methyl |
| C-303 | mexiletine | N | O | O | CONHn-Bu | O | O | 1 | methyl |
| C-304 | mexiletine | N | O | O | CONHn-Bu | O | O | 1 | C(CH₃)₃ |
| C-305 | mexiletine | N | O | O | CONHn-Bu | O | O | 1 | Ph |
| C-306 | mexiletine | N | O | O | CONHn-Bu | O | O | 1 | 4Me(Ph) |
| C-307 | mexiletine | N | O | O | CONHn-Bu | O | O | 1 | CF₃CH₂ |
| C-308 | mexiletine | N | O | O | CONHn-Bu | — | — | 0 | Cl |
| C-309 | mexiletine | N | O | O | CONHn-Bu | — | — | 0 | {N(Et)₃}⁺Cl⁻ |
| C-310 | ormetoprim | N | O | O | CONH—Bn | O | O | 1 | methyl |
| C-311 | prostaglandin E1 | O | O | O | CONH—Bn | O | O | 1 | C(CH₃)₃ |
| C-312 | quinacrine | N | O | O | CONH—Bn | — | — | 0 | {N(Et)₃}⁺Cl⁻ |
| C-313 | quinacrine | N | O | O | CONNH₂ | O | O | 1 | methyl |
| C-314 | quinacrine | N | O | O | CONHNHMe | O | O | 1 | methyl |
| C-315 | quinacrine | N | O | O | CONHNHEt | O | O | 1 | methyl |
| C-316 | quinacrine | N | O | O | CONHNHPr | O | O | 1 | methyl |
| C-317 | quinacrine | N | O | O | CONHNHt-Bu | O | O | 1 | C(CH₃)₃ |
| C-318 | quinacrine | N | O | O | CONHNHCO₂Me | O | O | 1 | C(CH₃)₃ |
| C-319 | quinacrine | N | O | O | CONHNHCO₂Et | O | O | 1 | C(CH₃)₃ |
| C-320 | quinacrine | N | O | O | CONHNHCO₂t-Bu | O | O | 1 | C(CH₃)₃ |
| C-321 | quinacrine | N | O | O | CONHNHCO₂Bn | O | O | 1 | C(CH₃)₃ |
| C-322 | quinacrine | N | O | O | CONHN═CHMe | O | O | 1 | methyl |
| C-323 | quinacrine | N | O | O | CONHN═CHMe | — | — | 0 | {N(Et)₃}⁺Cl⁻ |
| C-324 | quinacrine | N | O | O | CONHN═CHPh | O | O | 1 | methyl |
| C-325 | quinacrine | N | O | O | CONHN═CHPh | O | O | 1 | C(CH₃)₃ |
| C-326 | quinidine | N | O | O | CO₂H | O | O | 1 | methyl |
| C-327 | quinidine | N | O | O | CO₂H | O | O | 1 | C(CH₃)₃ |
| C-328 | quinidine | N | O | O | CO₂H | O | O | 1 | Ph |
| C-329 | quinidine | N | O | O | CO₂H | O | O | 1 | 4Me(Ph) |
| C-330 | quinidine | N | O | O | CO₂H | — | — | 0 | Cl |
| C-331 | quinidine | N | O | O | CO₂H | — | — | 0 | {N(Et)₃}⁺Cl⁻ |
| C-332 | quinidine | N | O | O | CO₂H | O | O | 0 | {N(Me)₂Ph}⁺Cl⁻ |
| C-333 | quinidine | N | O | O | CO₂Me | — | — | 1 | methyl |
| C-334 | quinidine | N | O | O | CO₂Me | O | O | 1 | C(CH₃)₃ |
| C-335 | quinidine | N | O | O | CO₂Me | O | O | 1 | Ph |
| C-336 | quinidine | N | O | O | CO₂Me | O | O | 1 | 4Me(Ph) |
| C-337 | quinidine | N | O | O | CO₂Me | — | — | 0 | Cl |
| C-338 | quinidine | N | O | O | CO₂Me | — | — | 0 | {N(Et)₃}⁺Cl⁻ |
| C-339 | quinidine | N | O | O | CO₂Me | — | — | 0 | {N(Me)₂Ph}⁺Cl⁻ |
| C-340 | quinidine | N | O | O | CO₂Et | O | O | 1 | methyl |
| C-341 | quinidine | N | O | O | CO₂Et | O | O | 0 | Cl |
| C-342 | quinidine | N | O | S | CO₂n-Pr | O | O | 1 | methyl |
| C-343 | quinidine | N | O | S | CO₂n-Pr | — | — | 0 | Cl |
| C-344 | quinidine | N | O | O | CO₂n-Bu | O | O | 1 | methyl |
| C-345 | sulfachlor- | N | O | O | CO₂n-Bu | O | O | 1 | C(CH₃)₃ |
| | pyridazine | | | | | | | | |
| C-346 | sulfadiazine | N | O | O | CO₂n-Bu | O | O | 1 | Ph |
| C-347 | sulfamethoxazole | N | O | S | CO₂n-Bu | O | O | 1 | 4Me(Ph) |
| C-348 | theophylline | N | O | S | CO₂n-Bu | O | O | 1 | CF₃CH₂ |
| C-349 | theophylline | N | O | O | CO₂n-Bu | — | — | 0 | Cl |
| C-350 | theophylline | N | O | O | CO₂n-Bu | — | — | 0 | {N(Et)₃}⁺Cl⁻ |
| C-351 | theophylline | N | O | O | CO₂n-Bu | — | — | 0 | {N(Me)₂Ph}⁺Cl⁻ |
| C-352 | theophylline | N | O | O | CO₂n-Bu | — | — | 0 | OC(CH₃)₃ |
| C-353 | theophylline | N | O | O | CO₂—Bn | O | O | 1 | methyl |
| C-354 | theophylline | N | O | O | CO₂—Bn | O | O | 1 | C(CH₃)₃ |
| C-355 | theophylline | N | O | O | CO₂—Bn | O | O | 1 | Ph |
| C-356 | theophylline | N | O | S | CO₂—Bn | O | O | 1 | 4Me(Ph) |
| C-357 | theophylline | N | O | S | CO₂—Bn | O | O | 1 | CF₃CH₂ |
| C-358 | theophylline | N | O | O | CO₂—Bn | — | — | 0 | Cl |
| C-359 | theophylline | N | O | O | CO₂—Bn | — | — | 0 | {N(Et)₃}⁺Cl⁻ |
| C-360 | theophylline | N | O | O | CO₂—Bn | — | — | 0 | {N(Me)₂Ph}⁺Cl⁻ |
| C-361 | theophylline | N | O | O | CO₂—Bn | O | O | 0 | OC(CH₃)₃ |
| C-362 | theophylline | N | O | O | COSMe | O | O | 1 | methyl |
| C-363 | theophylline | N | O | S | COSMe | O | O | 1 | C(CH₃)₃ |
| C-364 | theophylline | N | O | O | COSMe | — | — | 0 | Cl |
| C-365 | theophylline | N | O | O | COSMe | — | — | 0 | {N(Et)₃}⁺Cl⁻ |
| C-366 | theophylline | N | O | S | COSEt | O | O | 1 | methyl |
| C-367 | theophylline | N | O | O | COSEt | — | — | 0 | Cl |
| C-368 | theophylline | N | O | O | COSn-Bu | O | O | 1 | methyl |
| C-369 | theophylline | N | O | S | COSn-Bu | — | — | 0 | Cl |
| C-370 | theophylline | N | O | O | COSBn | O | O | 1 | methyl |
| C-371 | theophylline | N | O | O | COSBn | — | — | 0 | Cl |
| C-372 | theophylline | N | O | O | CONHMe | O | O | 1 | methyl |
| C-373 | theophylline | N | O | O | CONHMe | O | O | 1 | C(CH₃)₃ |
| C-374 | theophylline | N | O | O | CONHEt | O | O | 1 | methyl |
| C-375 | theophylline | N | O | O | CONHn-Pr | O | O | 1 | methyl |
| C-376 | theophylline | N | O | O | CONHn-Bu | O | O | 1 | methyl |
| C-377 | tiletamine | N | O | O | CONHn-Bu | O | O | 1 | C(CH₃)₃ |
| C-378 | tiletamine | N | O | O | CONHn-Bu | O | O | 1 | Ph |
| C-379 | tiletamine | N | O | O | CONHn-Bu | O | O | 1 | 4Me(Ph) |
| C-380 | tiletamine | N | O | O | CONHn-Bu | O | O | 1 | CF₃CH₂ |
| C-381 | tiletamine | N | O | O | CONHn-Bu | — | — | 0 | Cl |
| C-382 | tiletamine | N | O | O | CONHn-Bu | — | — | 0 | {N(Et)₃}⁺Cl⁻ |
| C-383 | tiletamine | N | O | O | CONH—Bn | O | O | 1 | methyl |
| C-384 | tiletamine | N | O | O | CONH—Bn | O | O | 1 | C(CH₃)₃ |
| C-385 | tiletamine | N | O | O | CONH—Bn | — | — | 0 | {N(Et)₃}⁺Cl⁻ |
| C-386 | tiletamine | N | O | O | CONNH₂ | O | O | 1 | methyl |
| C-387 | tiletamine | N | O | O | CONHNHMe | O | O | 1 | methyl |
| C-388 | tiletamine | N | O | O | CONHNHEt | O | O | 1 | methyl |
| C-389 | tiletamine | N | O | O | CONHNHPr | O | O | 1 | methyl |
| C-390 | tiletamine | N | O | O | CONHNHt-Bu | O | O | 1 | C(CH₃)₃ |
| C-391 | tiletamine | N | O | O | CONHNHCO₂Me | O | O | 1 | C(CH₃)₃ |
| C-392 | tiletamine | N | O | O | CONHNHCO₂Et | O | O | 1 | C(CH₃)₃ |
| C-393 | tiletamine | N | O | O | CONHNHCO₂t-Bu | O | O | 1 | C(CH₃)₃ |
| C-394 | tiletamine | N | O | O | CONHNHCO₂Bn | O | O | 1 | C(CH₃)₃ |
| C-395 | tiletamine | N | O | O | CONHN═CHMe | O | O | 1 | methyl |
| C-396 | tiletamine | N | O | O | CONHN═CHMe | — | — | 0 | {N(Et)₃}⁺Cl⁻ |
| C-397 | tiletamine | N | O | O | CONHN═CHPh | O | O | 1 | methyl |
| C-398 | tiletamine | N | O | O | CONHN═CHPh | O | O | 1 | C(CH₃)₃ |
| C-399 | tacainide | N | O | O | CO₂H | O | O | 1 | methyl |
| C-400 | tocainide | N | O | O | CO₂H | O | O | 1 | C(CH₃)₃ |
| C-401 | tocainide | N | O | O | CO₂H | O | O | 1 | Ph |
| C-402 | tocainide | N | O | O | CO₂H | O | O | 1 | 4Me(Ph) |
| C-403 | tocainide | N | O | O | CO₂H | — | — | 0 | Cl |
| C-404 | tocainide | N | O | O | CO₂H | — | — | 0 | {N(Et)₃}⁺Cl⁻ |
| C-405 | tocainide | N | O | O | CO₂H | — | — | 0 | {N(Me)₂Ph}⁺Cl⁻ |
| C-406 | tocainide | N | O | O | CO₂Me | O | O | 1 | methyl |
| C-407 | tocainide | N | O | O | CO₂Me | O | O | 1 | C(CH₃)₃ |
| C-408 | tocainide | N | O | O | CO₂Me | O | O | 1 | Ph |
| C-409 | tocainide | N | O | O | CO₂Me | O | O | 1 | 4Me(Ph) |
| C-410 | tocainide | N | O | O | CO₂Me | — | — | 0 | Cl |
| C-411 | tocainide | N | O | O | CO₂Me | — | — | 0 | {N(Et)₃}⁺Cl⁻ |
| C-412 | tocainide | N | O | O | CO₂Me | — | — | 0 | {N(Me)₂Ph}⁺Cl⁻ |
| C-413 | tocainide | N | O | O | CO₂Et | O | O | 1 | methyl |
| C-414 | tocainide | N | O | O | CO₂Et | — | — | 0 | Cl |
| C-415 | tocainide | N | O | S | CO₂n-Pr | O | O | 1 | methyl |
| C-416 | tocainide | N | O | S | CO₂n-Pr | — | — | 0 | Cl |
| C-417 | tocainide | N | O | O | CO₂n-Bu | O | O | 1 | methyl |
| C-418 | tocainide | N | O | O | CO₂n-Bu | O | O | 1 | C(CH₃)₃ |
| C-419 | tocainide | N | O | O | CO₂n-Bu | O | O | 1 | Ph |
| C-420 | tocainide | N | O | S | CO₂n-Bu | O | O | 1 | 4Me(Ph) |
| C-421 | tocainide | N | O | S | CO₂n-Bu | O | O | 1 | CF₃CH₂ |
| C-422 | tocainide | N | O | O | CO₂n-Bu | — | — | 0 | Cl |
| C-423 | tocainide | N | O | O | CO₂n-Bu | — | — | 0 | {N(Et)₃}⁺Cl⁻ |
| C-424 | tocainide | N | O | O | CO₂n-Bu | — | — | 0 | {N(Me)₂Ph}⁺Cl⁻ |
| C-425 | tocainide | N | O | O | CO₂n-Bu | — | — | 0 | OC(CH₃)₃ |
| C-426 | vincristine | N | O | O | CO₂—Bn | O | O | 1 | methyl |
| C-427 | xylazine | N | O | O | CO₂—Bn | O | O | 1 | C(CH₃)₃ |
| C-428 | xylazine | N | O | O | CO₂—Bn | O | O | 1 | Ph |
| C-429 | xylazine | N | O | S | CO₂—Bn | O | O | 1 | 4Me(Ph) |
| C-430 | xylazine | N | O | S | CO₂—Bn | O | O | 1 | CF₃CH₂ |
| C-431 | xylazine | N | O | O | CO₂—Bn | — | — | 0 | Cl |
| C-432 | xylazine | N | O | O | CO₂—Bn | — | — | 0 | {N(Et)₃}⁺Cl⁻ |
| C-433 | xylazine | N | O | O | CO₂—Bn | — | — | 0 | {N(Me)₂Ph}⁺Cl⁻ |
| C-434 | xylazine | N | O | O | CO₂—Bn | — | — | 0 | OC(CH₃)₃ |
| C-435 | xylazine | N | O | O | COSMe | O | O | 1 | methyl |
| C-436 | xylazine | N | O | S | COSMe | O | O | 1 | C(CH₃)₃ |
| C-437 | xylazine | N | O | O | COSMe | — | — | 0 | Cl |
| C-438 | xylazine | N | O | O | COSMe | — | — | 0 | {N(Et)₃}⁺Cl⁻ |
| C-439 | xylazine | N | O | S | COSEt | O | O | 1 | methyl |
| C-440 | xylazine | N | O | O | COSEt | — | — | 0 | Cl |
| C-441 | xylazine | N | O | O | COSn-Bu | O | O | 1 | methyl |
| C-442 | xylazine | N | O | S | COSn-Bu | — | — | 0 | Cl |
| C-443 | xylazine | N | O | O | COSBn | O | O | 1 | methyl |
| C-444 | xylazine | N | O | O | COSBn | — | — | 0 | Cl |
| C-445 | xylazine | N | O | O | CONHMe | O | O | 1 | methyl |
| C-446 | xylazine | N | O | O | CONHMe | O | O | 1 | C(CH₃)₃ |
| C-447 | xylazine | N | O | O | CONHEt | O | O | 1 | methyl |
| C-448 | xylazine | N | O | O | CONHn-Pr | O | O | 1 | methyl |
| C-449 | xylazine | N | O | O | CONHn-Bu | O | O | 1 | methyl |
| C-450 | xylazine | N | O | O | CONHn-Bu | O | O | 1 | C(CH₃)₃ |
| C-451 | xylazine | N | O | O | CONHn-Bu | O | O | 1 | Ph |
| C-452 | xylazine | N | O | O | CONHn-Bu | O | O | 1 | 4Me(Ph) |
| C-453 | xylazine | N | O | O | CONHn-Bu | O | O | 1 | CF₃CH₂ |
| C-454 | xylazine | N | O | O | CONHn-Bu | — | — | 0 | Cl |
| C-455 | xylazine | N | O | O | CONHn-Bu | — | — | 0 | {N(Et)₃}⁺Cl⁻ |
| C-456 | xylazine | N | O | O | CONH—Bn | O | O | 1 | methyl |
| C-457 | xylazine | N | O | O | CONH—Bn | O | O | 1 | C(CH₃)₃ |
| C-458 | xylazine | N | O | O | CONH—Bn | — | — | 0 | {N(Et)₃}⁺Cl⁻ |
| C-459 | xylazine | N | O | O | CONNH₂ | O | O | 1 | methyl |
| C-460 | xylazine | N | O | O | CONHNHMe | O | O | 1 | methyl |
| C-461 | xylazine | N | O | O | CONHNHEt | O | O | 1 | methyl |
| C-462 | xylazine | N | O | O | CONHNHPr | O | O | 1 | methyl |
| C-463 | xylazine | N | O | O | CONHNHt-Bu | O | O | 1 | C(CH₃)₃ |
| C-464 | xylazine | N | O | O | CONHNHCO₂Me | O | O | 1 | C(CH₃)₃ |
| C-465 | xylazine | N | O | O | CONHNHCO₂Et | O | O | 1 | C(CH₃)₃ |
| C-466 | xylazine | N | O | O | CONHNHCO₂t-Bu | O | O | 1 | C(CH₃)₃ |
| C-467 | xylazine | N | O | O | CONHNHCO₂Bn | O | O | 1 | C(CH₃)₃ |
| C-468 | xylazine | N | O | O | CONHN═CHMe | O | O | 1 | methyl |
| C-469 | xylazine | N | O | O | CONHN═CHMe | — | — | 0 | {N(Et)₃}⁺Cl⁻ |
| C-470 | xylazine | N | O | O | CONHN═CHPh | O | O | 1 | methyl |
| C-471 | xylazine | N | O | O | CONHN═CHPh | O | O | 1 | C(CH₃)₃ |

Table D describes additional examples of compounds of Formula I which can be made using the procedures described hereinbefore, where R² is hydrogen, m=0, q=1, t=0 or 1 and the pharmaceutical (a veterinary drug) which defines the 5 pharmaceutical moiety of these examples is Z²(X²)—[(C=G²0)-G²¹]-H, Z²(X²)_{q}—H or Z²(X²).). The following groups, Z¹, G¹⁰, G¹¹, R¹, G²⁰, G²¹, t, X² and Z²(X²)—[(C=G²⁰)-G²¹]-H, Z²(X²)_{q}—H or Z²(X²) are defined within Table D.

TABLE D

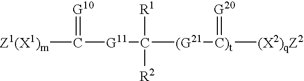

| | | | | | | | | | Z²(X²)_{q}-[(C═G²⁰)-G²¹]ₜ-H |
|---|---|---|---|---|---|---|---|---|---|
| | | | | | | | | | or when t = 0, Z²(X²)_{q} or |
| Cmpd # | Z¹ | G¹⁰ | G¹¹ | R¹ | G²⁰ | G²¹ | t | X² | Z²(X²)_{q}-H |
| D-1 | methyl | O | O | CO₂H | — | — | 0 | N | albendazole |
| D-2 | methyl | O | O | CO₂H | — | — | 0 | O | clioquinol |
| D-3 | n-propyl | O | O | CO₂H | — | — | 0 | N | febantel |
| D-4 | n-propyl | O | O | CO₂H | — | — | 0 | N | fenbendazole |
| D-5 | C(CH₃)₃ | O | O | CO₂H | — | — | 0 | O | guaifenesin |
| D-6 | C(CH₃)₃ | O | O | CO₂H | — | — | 0 | O | mibolerone |
| D-7 | Ph | O | O | CO₂H | — | — | 0 | N | omeprazole |
| D-8 | Ph | O | O | CO₂Me | — | — | 0 | O | oxazepam |
| D-9 | 4Cl(Ph) | O | O | CO₂Me | — | — | 0 | N | piroxicam |
| D-10 | 4Cl(Ph) | O | O | CO₂Me | — | — | 0 | N | primidone |
| D-11 | methyl | O | O | CO₂Me | — | — | 0 | N | procainamide |
| D-12 | methyl | O | O | CO₂Me | — | — | 0 | N | thiabendazole |
| D-13 | n-propyl | O | O | CO₂Me | — | — | 0 | O | warfarin |
| D-14 | n-propyl | O | O | CO₂Me | — | — | 0 | N | albendazole |
| D-15 | Ph | O | O | CO₂Et | — | — | 0 | O | clioquinol |
| D-16 | Ph | O | O | CO₂Et | — | — | 0 | N | febantel |
| D-17 | methyl | O | S | CO₂n-Pr | — | — | 0 | N | fenbendazole |
| D-18 | n-propyl | O | S | CO₂n-Pr | — | — | 0 | O | guaifenesin |
| D-19 | C(CH₃)₃ | O | O | CO₂n-Bu | — | — | 0 | O | mibolerone |
| D-20 | Ph | O | O | CO₂n-Bu | — | — | 0 | N | omeprazole |
| D-21 | PhO | O | O | CO₂n-Bu | — | — | 0 | O | oxazepam |
| D-22 | PhNH | O | S | CO₂n-Bu | — | — | 0 | N | piroxicam |
| D-23 | 4Cl(Ph) | O | S | CO₂n-Bu | — | — | 0 | N | primidone |
| D-24 | 4Cl(Ph)O | O | O | CO₂n-Bu | — | — | 0 | N | procainamide |
| D-25 | 4Cl(Ph)NH | O | O | CO₂n-Bu | — | — | 0 | N | thiabendazole |
| D-26 | pyrid-2-yl | O | O | CO₂n-Bu | — | — | 0 | O | warfarin |
| D-27 | pyrid-4-yl | O | O | CO₂n-Bu | — | — | 0 | N | albendazole |
| D-28 | methyl | O | O | CO₂—Bn | — | — | 0 | O | clioquinol |
| D-29 | n-propyl | O | O | CO₂—Bn | — | — | 0 | N | febantel |
| D-30 | C(CH₃)₃ | O | O | CO₂—Bn | — | — | 0 | N | fenbendazole |
| D-31 | Ph | O | S | CO₂—Bn | — | — | 0 | O | guaifenesin |
| D-32 | PhO | O | S | CO₂—Bn | — | — | 0 | O | mibolerone |
| D-33 | PhNH | O | O | CO₂—Bn | — | — | 0 | N | omeprazole |
| D-34 | 4Cl(Ph) | O | O | CO₂—Bn | — | — | 0 | O | oxazepam |
| D-35 | 4Cl(Ph)O | O | O | CO₂—Bn | — | — | 0 | N | piroxicam |
| D-36 | 4Cl(Ph)NH | O | O | CO₂—Bn | — | — | 0 | N | primidone |
| D-37 | pyrid-2-yl | O | O | COSMe | — | — | 0 | N | procainamide |
| D-38 | pyrid-4-yl | O | S | COSMe | — | — | 0 | N | thiabendazole |
| D-39 | methyl | O | O | COSMe | — | — | 0 | O | warfarin |
| D-40 | n-propyl | O | O | COSMe | — | — | 0 | N | albendazole |
| D-41 | C(CH₃)₃ | O | S | COSEt | — | — | 0 | O | clioquinol |
| D-42 | Ph | O | O | COSEt | — | — | 0 | N | febantel |
| D-43 | PhO | O | O | COSn-Bu | — | — | 0 | N | fenbendazole |
| D-44 | Ph—NH | O | S | COSn-Bu | — | — | 0 | O | guaifenesin |
| D-45 | 4Cl(Ph) | O | O | COSBn | — | — | 0 | O | mibolerone |
| D-46 | 4Cl(Ph)O | O | O | COSBn | — | — | 0 | N | omeprazole |
| D-47 | 4Cl(Ph)NH | O | O | CONHMe | — | — | 0 | O | oxazepam |
| D-48 | pyrid-2-yl | O | O | CONHMe | — | — | 0 | N | piroxicam |
| D-49 | pyrid-4-yl | O | O | CONHEt | — | — | 0 | N | primidone |
| D-50 | methyl | O | O | CONHn-Pr | — | — | 0 | N | procainamide |
| D-51 | n-propyl | O | O | CONHn-Bu | — | — | 0 | N | thiabendazole |
| D-52 | C(CH₃)₃ | O | O | CONHn-Bu | — | — | 0 | O | warfarin |
| D-53 | Ph | O | O | CONHn-Bu | — | — | 0 | N | albendazole |
| D-54 | PhO | O | O | CONHn-Bu | — | — | 0 | O | clioquinol |
| D-55 | Ph—NH | O | O | CONHn-Bu | — | — | 0 | N | febantel |
| D-56 | 4C1(Ph) | O | O | CONHn-Bu | — | — | 0 | N | fenbendazole |
| D-57 | 4Cl(Ph)O | O | O | CONHn-Bu | — | — | 0 | O | guaifenesin |
| D-58 | 4Cl(Ph)NH | O | O | CONH—Bn | — | — | 0 | O | mibolerone |
| D-59 | pyrid-2-yl | O | O | CONH—Bn | — | — | 0 | N | omeprazole |
| D-60 | pyrid-4-yl | O | O | CONH—Bn | — | — | 0 | O | oxazepam |
| D-61 | methyl | O | O | CONNH₂ | — | — | 0 | N | piroxicam |
| D-62 | n-propyl | O | O | CONHNHMe | — | — | 0 | N | primidone |
| D-63 | C(CH₃)₃ | O | O | CONHNHEt | — | — | 0 | N | procainamide |
| D-64 | Ph | O | O | CONHNHPr | — | — | 0 | N | thiabendazole |
| D-65 | PhO | O | O | CONHNHt-Bu | — | — | 0 | O | warfarin |
| D-66 | Ph—NH | O | O | CONHNHCO₂Me | — | — | 0 | N | albendazole |
| D-67 | 4Cl(Ph) | O | O | CONHNHCO₂Et | — | — | 0 | O | clioquinol |
| D-68 | 4Cl(Ph)O | O | O | CONHNHCO₂t-Bu | — | — | 0 | N | febantel |
| D-69 | 4Cl(Ph)NH | O | O | CONHNHCO₂Bn | — | — | 0 | N | fenbendazole |
| D-70 | pyrid-2-yl | O | O | CONHN═CHMe | — | — | 0 | O | guaifenesin |
| D-71 | pyrid-4-yl | O | O | CONHN═CHMe | — | — | 0 | O | mibolerone |
| D-72 | methyl | O | O | CONHN═CHPh | — | — | 0 | N | omeprazole |
| D-73 | n-propyl | O | O | CONHN═CHPh | — | — | 0 | O | oxazepam |
| D-74 | methyl | O | O | CO₂H | O | O | 1 | C | captopril |
| D-75 | ethyl | O | O | CO₂H | O | O | 1 | C | captopril |
| D-76 | n-propyl | O | O | CO₂H | O | O | 1 | C | captopril |
| D-77 | C(CH₃)₃ | O | O | CO₂H | O | O | 1 | C | captopril |
| D-78 | MeO | O | O | CO₂H | O | O | 1 | C | captopril |
| D-79 | EtO | O | O | CO₂H | O | O | 1 | C | captopril |
| D-80 | n-PrO | O | O | CO₂H | O | O | 1 | C | captopril |
| D-81 | MeNH | O | O | CO₂Me | O | O | 1 | C | captopril |
| D-82 | EtNH | O | O | CO₂Me | O | O | 1 | C | captopril |
| D-83 | n-PrNH | O | O | CO₂Me | O | O | 1 | C | captopril |
| D-84 | n-PrO | O | O | CO₂Me | O | O | 1 | C | captopril |
| D-85 | PhO | O | O | CO₂Me | O | O | 1 | C | captopril |
| D-86 | Ph—NH | O | O | CO₂Me | O | O | 1 | C | captopril |
| D-87 | 4Cl(Ph) | O | O | CO₂Me | O | O | 1 | C | captopril |
| D-88 | 4Cl(Ph)O | O | O | CO₂Et | O | O | 1 | C | captopril |
| D-89 | 4Cl(Ph)NH | O | O | CO₂Et | O | O | 1 | C | captopril |
| D-90 | MeO | O | S | CO₂n-Pr | O | O | 1 | C | captopril |
| D-91 | EtO | O | S | CO₂n-Pr | O | O | 1 | C | captopril |
| D-92 | n-PrO | O | O | CO₂n-Bu | O | O | 1 | C | captopril |
| D-93 | MeNH | O | O | CO₂n-Bu | O | O | 1 | C | captopril |
| D-94 | EtNH | O | O | CO₂n-Bu | O | O | 1 | C | captopril |
| D-95 | n-PrNH | O | S | CO₂n-Bu | O | O | 1 | C | captopril |
| D-96 | n-PrO | O | S | CO₂n-Bu | O | O | 1 | C | captopril |
| D-97 | PhO | O | O | CO₂n-Bu | O | O | 1 | C | captopril |
| D-98 | Ph—NH | O | O | CO₂n-Bu | O | O | 1 | C | captopril |
| D-99 | 4Cl(Ph) | O | O | CO₂n-Bu | O | O | 1 | C | captopril |
| D-100 | 4Cl(Ph)O | O | O | CO₂n-Bu | O | O | 1 | C | captopril |
| D-101 | MeO | O | O | CO₂—Bn | O | O | 1 | C | cephapirin |
| D-102 | EtO | O | O | CO₂—Bn | O | O | 1 | C | cephapirin |
| D-103 | n-PrO | O | O | CO₂—Bn | O | O | 1 | C | cephapirin |
| D-104 | MeNH | O | S | CO₂—Bn | O | O | 1 | C | cephapirin |
| D-105 | EtNH | O | S | CO₂—Bn | O | O | 1 | C | cephapirin |
| D-106 | n-PrNH | O | O | CO₂—Bn | O | O | 1 | C | cephapirin |
| D-108 | PhO | O | O | CO₂—Bn | O | O | 1 | C | cephapirin |
| D-109 | Ph—NH | O | O | CO₂—Bn | O | O | 1 | C | cephapirin |
| D-110 | 4Cl(Ph) | O | O | COSMe | O | O | 1 | C | cephapirin |
| D-111 | 4Cl(Ph)O | O | S | COSMe | O | O | 1 | C | cephapirin |
| D-112 | 4Cl(Ph)NH | O | O | COSMe | O | O | 1 | C | cephapirin |
| D-113 | MeO | O | O | COSMe | O | O | 1 | C | cephapirin |
| D-114 | EtO | O | S | COSEt | O | O | 1 | C | cephapirin |
| D-115 | n-PrO | O | O | COSEt | O | O | 1 | C | cephapirin |
| D-116 | MeNH | O | O | COSn-Bu | O | O | 1 | C | cephapirin |
| D-117 | EtNH | O | S | COSn-Bu | O | O | 1 | C | cephapirin |
| D-118 | n-PrNH | O | O | COSBn | O | O | 1 | C | cephapirin |
| D-119 | n-PrO | O | O | COSBn | O | O | 1 | C | cephapirin |
| D-120 | PhO | O | O | CONHMe | O | O | 1 | C | cephapirin |
| D-121 | PhNH | O | O | CONHMe | O | O | 1 | C | cephapirin |
| D-122 | 4Cl(Ph) | O | O | CONHEt | O | O | 1 | C | cephapirin |
| D-123 | 4Cl(Ph)O | O | O | CONHn-Pr | O | O | 1 | C | cephapirin |
| D-124 | 4Cl(Ph)NH | O | O | CONHn-Bu | O | O | 1 | C | cephapirin |
| D-125 | MeO | O | O | CONHn-Bu | O | O | 1 | C | cephapirin |
| D-126 | EtO | O | O | CONHn-Bu | O | O | 1 | C | cephapirin |
| D-127 | n-PrO | O | O | CONHn-Bu | O | O | 1 | C | cephapirin |
| D-128 | MeNH | O | O | CONHn-Bu | O | O | 1 | C | cephapirin |
| D-129 | EtNH | O | O | CONHn-Bu | O | O | 1 | C | cephapirin |
| D-130 | n-PrNH | O | O | CONHn-Bu | O | O | 1 | C | cephapirin |
| D-131 | n-PrO | O | O | CONH—Bn | O | O | 1 | C | cephapirin |
| D-132 | PhO | O | O | CONH—Bn | O | O | 1 | C | cephapirin |
| D-133 | Ph—NH | O | O | CONH—Bn | O | O | 1 | C | cephapirin |
| D-134 | 4Cl(Ph) | O | O | CONNH₂ | O | O | 1 | C | cephapirin |
| D-135 | 4Cl(Ph)O | O | O | CONHNHMe | O | O | 1 | C | cephapirin |
| D-136 | 4Cl(Ph)NH | O | O | CONHNHEt | O | O | 1 | C | cephapirin |
| D-137 | MeO | O | O | CONHNHPr | O | O | 1 | C | cephapirin |
| D-138 | EtO | O | O | CONHNHt-Bu | O | O | 1 | C | cephapirin |
| D-139 | n-PrO | O | O | CONHNHCO₂Me | O | O | 1 | C | cephapirin |
| D-140 | MeNH | O | O | CONHNHCO₂Et | O | O | 1 | C | cephapirin |
| D-141 | EtNH | O | O | CONHNHCO₂t-Bu | O | O | 1 | C | cephapirin |
| D-142 | n-PrNH | O | O | CONHNHCO₂Bn | O | O | 1 | C | cephapirin |
| D-143 | n-PrO | O | O | CONHN═CHMe | O | O | 1 | C | cephapirin |
| D-144 | PhO | O | O | CONHN═CHMe | O | O | 1 | C | cephapirin |
| D-145 | Ph—NH | O | O | CONHN═CHPh | O | O | 1 | C | cephapirin |
| D-146 | 4Cl(Ph)NH | O | O | CONHN═CHPh | O | O | 1 | C | cephapirin |
| D-147 | methyl | O | O | CO₂H | — | — | 0 | N | cephapirin |
| D-148 | ethyl | O | O | CO₂H | — | — | 0 | N | cephapirin |
| D-149 | n-propyl | O | O | CO₂H | — | — | 0 | N | cephapirin |
| D-150 | C(CH₃)₃ | O | O | CO₂H | — | — | 0 | N | cephapirin |
| D-151 | MeO | O | O | CO₂H | — | — | 0 | N | cephapirin |
| D-152 | EtO | O | O | CO₂H | — | — | 0 | N | cephapirin |
| D-153 | n-PrO | O | O | CO₂H | — | — | 0 | N | cephapirin |
| D-154 | MeNH | O | O | CO₂Me | — | — | 0 | N | cephapirin |
| D-155 | EtNH | O | O | CO₂Me | — | — | 0 | N | cephapirin |
| D-156 | n-PrNH | O | O | CO₂Me | — | — | 0 | N | cephapirin |
| D-157 | n-PrO | O | O | CO₂Me | — | — | 0 | N | cephapirin |
| D-158 | PhO | O | O | CO₂Me | — | — | 0 | N | cephapirin |
| D-159 | PhNH | O | O | CO₂Me | — | — | 0 | N | cephapirin |
| D-160 | 4Cl(Ph) | O | O | CO₂Me | — | — | 0 | N | cephapirin |
| D-161 | 4Cl(Ph)O | O | O | CO₂Et | — | — | 0 | N | cephapiirin |
| D-162 | 4Cl(Ph)NH | O | O | CO₂Et | — | — | 0 | N | cephapirin |
| D-163 | Ph—NH | O | S | CO₂n-Pr | — | — | 0 | N | cephapirin |
| D-164 | 4Cl(Ph) | O | S | CO₂n-Pr | — | — | 0 | N | cephapirin |
| D-165 | 4Cl(Ph)O | O | O | CO₂n-Bu | — | — | 0 | N | cephapirin |
| D-166 | 4Cl(Ph)NH | O | O | CO₂n-Bu | — | — | 0 | N | cephapirin |
| D-167 | pyrid-2-yl | O | O | CO₂n-Bu | — | — | 0 | N | cephapirin |
| D-168 | pyrid-4-yl | O | S | CO₂n-Bu | — | — | 0 | N | cephapirin |
| D-169 | MeO | O | S | CO₂n-Bu | — | — | 0 | N | cephapirin |
| D-170 | EtO | O | O | CO₂n-Bu | — | — | 0 | N | cephapirin |
| D-171 | n-PrO | O | O | CO₂n-Bu | — | — | 0 | N | cephapirin |
| D-172 | PhO | O | O | CO₂n-Bu | — | — | 0 | N | cephapirin |
| D-173 | MeO | O | O | CO₂n-Bu | O | O | 1 | C | ketoprofen |
| D-174 | EtO | O | O | CO₂—Bn | O | O | 1 | C | ketoprofen |
| D-175 | n-PrO | O | O | CO₂—Bn | O | O | 1 | C | ketoprofen |
| D-176 | (CH₃)₃CO | O | O | CO₂—Bn | O | O | 1 | C | ketoprofen |
| D-177 | MeNH | O | S | CO₂—Bn | O | O | 1 | C | ketoprofen |
| D-178 | EtNH | O | S | CO₂—Bn | O | O | 1 | C | ketoprofen |
| D-179 | n-PrNH | O | O | CO₂—Bn | O | O | 1 | C | ketoprofen |
| D-180 | (CH₃)₃CNH | O | O | CO₂—Bn | O | O | 1 | C | ketoprofen |
| D-181 | PhO | O | O | CO₂—Bn | O | O | 1 | C | ketoprofen |
| D-182 | PhNH | O | O | CO₂—Bn | O | O | 1 | C | ketoprofen |
| D-183 | 4Cl(Ph) | O | O | COSMe | O | O | 1 | C | ketaprofen |
| D-184 | 4Cl(Ph)O | O | S | COSMe | O | O | 1 | C | ketoprofen |
| D-185 | 4Cl(Ph)NH | O | O | COSMe | O | O | 1 | C | ketoprofen |
| D-186 | MeO | O | O | COSMe | O | O | 1 | C | ketoprofen |
| D-187 | EtO | O | S | COSEt | O | O | 1 | C | ketoprofen |
| D-188 | n-PrO | O | O | COSEt | O | O | 1 | C | ketoprofen |
| D-189 | (CH₃)₃CO | O | O | COSn-Bu | O | O | 1 | C | ketoprofen |
| D-190 | MeNH | O | S | COSn-Bu | O | O | 1 | C | ketoprofen |
| D-191 | EtNH | O | O | COSBn | O | O | 1 | C | ketoprofen |
| D-192 | n-PrNH | O | O | COSBn | O | O | 1 | C | ketoprofen |
| D-193 | (CH₃)₃CNH | O | O | CONHMe | O | O | 1 | C | ketoprofen |
| D-194 | PhO | O | O | CONHMe | O | O | 1 | C | ketoprofen |
| D-195 | Ph—NH | O | O | CONHEt | O | O | 1 | C | ketoprofen |
| D-196 | 4Cl(Ph) | O | O | CONHn-Pr | O | O | 1 | C | ketoprofen |
| D-197 | 4Cl(Ph)O | O | O | CONHn-Bu | O | O | 1 | C | ketoprofen |
| D-198 | 4Cl(Ph)NH | O | O | CONHn-Bu | O | O | 1 | C | ketoprofen |
| D-199 | MeO | O | O | CONHn-Bu | O | O | 1 | C | ketoprofen |
| D-200 | EtO | O | O | CONHn-Bu | O | O | 1 | C | ketoprofen |
| D-201 | n-PrO | O | O | CONHn-Bu | O | O | 1 | C | ketoprofen |
| D-202 | (CH₃)₃CO | O | O | CONHn-Bu | O | O | 1 | C | ketoprofen |
| D-203 | MeNH | O | O | CONHn-Bu | O | O | 1 | C | ketoprofen |
| D-204 | EtNH | O | O | CONH—Bn | O | O | 1 | C | ketoprofen |
| D-205 | n-PrNH | O | O | CONH—Bn | O | O | 1 | C | ketoprofen |
| D-206 | (CH₃)₃CNH | O | O | CONH—Bn | O | O | 1 | C | ketoprofen |
| D-207 | PhO | O | O | CONNH₂ | O | O | 1 | C | ketoprofen |
| D-208 | Ph—NH | O | O | CONHNHMe | O | O | 1 | C | ketoprofen |
| D-209 | 4Cl(Ph) | O | O | CONHNHEt | O | O | 1 | C | ketoprofen |
| D-210 | 4Cl(Ph)O | O | O | CONHNHPr | O | O | 1 | C | ketoprofen |
| D-211 | 4CH₃O(Ph)O | O | O | CONHNHt-Bu | O | O | 1 | C | ketoprofen |
| D-212 | 4NO₂(Ph)O | O | O | CONHNHCO₂Me | O | O | 1 | C | ketoprofen |
| D-213 | 4Cl(Ph)NH | O | O | CONHNHCO₂Et | O | O | 1 | C | ketoprofen |
| D-214 | 4MeO(Ph)NH | O | O | CONHNHCO₂t-Bu | O | O | 1 | C | ketoprofen |
| D-215 | MeO | O | O | CONHNHCO₂Bn | O | O | 1 | C | ketoprofen |
| D-216 | EtO | O | O | CONHN═CHMe | O | O | 1 | C | ketoprofen |
| D-217 | n-PrO | O | O | CONHN═CHMe | O | O | 1 | C | ketoprofen |
| D-218 | (CH₃)₃CO | O | O | CONHN═CHPh | O | O | 1 | C | ketoprofen |
| D-219 | MeNH | O | O | CONHN═CHPh | O | O | 1 | C | ketoprofen |
| D-220 | EtNH | O | O | CO₂H | O | O | 1 | C | ketoprofen |
| D-221 | n-PrNH | O | O | CO₂H | O | O | 1 | C | ketoprofen |
| D-222 | (CH₃)₃CNH | O | O | CO₂H | O | O | 1 | C | ketoprofen |
| D-223 | PhO | O | O | CO₂H | O | O | 1 | C | ketoprofen |
| D-224 | PhNH | O | O | CO₂H | O | O | 1 | C | ketoprofen |
| D-225 | 4Cl(Ph) | O | O | CO₂H | O | O | 1 | C | ketoprofen |
| D-226 | 4Cl(Ph)O | O | O | CO₂H | O | O | 1 | C | ketoprofen |
| D-227 | 4CH₃O(Ph)O | O | O | CO₂Me | O | O | 1 | C | ketoprofen |
| D-228 | 4NO₂(Ph)O | O | O | CO₂Me | O | O | 1 | C | ketoprofen |
| D-229 | 4Cl(Ph)NH | O | O | CO₂Me | O | O | 1 | C | ketoprofen |
| D-230 | 4MeO(Ph)NH | O | O | CO₂Me | O | O | 1 | C | ketoprofen |
| D-231 | PhNH | O | O | CO₂Me | O | O | 1 | C | ketoprofen |
| D-234 | 4Cl(Ph)NH | O | O | CO₂Et | O | O | 1 | C | ketoprofen |
| D-235 | 4MeO(Ph)NH | O | O | CO₂Et | O | O | 1 | C | ketoprofen |
| D-236 | PhO | O | S | CO₂n-Pr | O | O | 1 | C | ketoprofen |
| D-237 | PhNH | O | S | CO₂n-Pr | O | O | 1 | C | ketoprofen |
| D-238 | methyl | O | O | CO₂n-Bu | O | O | 1 | C | ketoprofen |
| D-239 | ethyl | O | O | CO₂n-Bu | O | O | 1 | C | ketoprofen |
| D-240 | n-propyl | O | O | CO₂n-Bu | O | O | 1 | C | ketoprofen |
| D-241 | C(CH₃)₃ | O | S | CO₂n-Bu | O | O | 1 | C | ketoprofen |
| D-242 | MeO | O | S | CO₂n-Bu | O | O | 1 | C | ketoprofen |
| D-243 | EtO | O | O | CO₂n-Bu | O | O | 1 | C | ketoprofen |
| D-244 | n-PrO | O | O | CO₂n-Bu | O | O | 1 | C | ketoprofen |
| D-245 | MeNH | O | O | CO₂n-Bu | O | O | 1 | C | ketoprofen |
| D-246 | EtNH | O | O | CO₂n-Bu | O | O | 1 | C | ketoprofen |
| D-247 | n-PrNH | O | O | CO₂—Bn | O | O | 1 | C | valproic acid |
| D-248 | n-PrO | O | O | CO₂—Bn | O | O | 1 | C | valproic acid |
| D-249 | PhO | O | O | CO₂—Bn | O | O | 1 | C | valproic acid |
| D-250 | Ph—NH | O | S | CO₂—Bn | O | O | 1 | C | valproic acid |
| D-251 | 4Cl(Ph) | O | S | CO₂—Bn | O | O | 1 | C | valproic acid |
| D-252 | 4Cl(Ph)O | O | O | CO₂—Bn | O | O | 1 | C | valproic acid |
| D-253 | 4CH₃O(Ph)O | O | O | CO₂—Bn | O | O | 1 | C | valproic acid |
| D-254 | 4NO₂(Ph)O | O | O | CO₂—Bn | O | O | 1 | C | valproic acid |
| D-255 | 4Cl(Ph)NH | O | O | CO₂—Bn | O | O | 1 | C | valproic acid |
| D-256 | 4F(Ph)NH | O | O | COSMe | O | O | 1 | C | valproic acid |
| D-257 | 4MeO(Ph)NH | O | S | COSMe | O | O | 1 | C | valproic acid |
| D-258 | pyrid-2-yl | O | O | COSMe | O | O | 1 | C | valproic acid |
| D-259 | pyrid-4-yl | O | O | COSMe | O | O | 1 | C | valproic acid |
| D-260 | MeO | O | S | COSEt | O | O | 1 | C | valproic acid |
| D-261 | EtO | O | O | COSEt | O | O | 1 | C | valproic acid |
| D-262 | n-PrO | O | O | COSn-Bu | O | O | 1 | C | valproic acid |
| D-263 | PhO | O | S | COSn-Bu | O | O | 1 | C | valproic acid |
| D-264 | MeNH | O | O | COSBn | O | O | 1 | C | valproic acid |
| D-265 | PhNH | O | O | COSBn | O | O | 1 | C | valproic acid |
| D-266 | MeO | O | O | CONHMe | O | O | 1 | C | valproic acid |
| D-267 | EtO | O | O | CONHMe | O | O | 1 | C | valproic acid |
| D-268 | n-PrO | O | O | CONHEt | O | O | 1 | C | valproic acid |
| D-269 | (CH₃)₃CO | O | O | CONHn-Pr | O | O | 1 | C | valproic acid |
| D-270 | MeNH | O | O | CONHn-Bu | O | O | 1 | C | valproic acid |
| D-271 | EtNH | O | O | CONHn-Bu | O | O | 1 | C | valproic acid |
| D-272 | n-PrNH | O | O | CONHn-Bu | O | O | 1 | C | valproic acid |
| D-273 | (CH₃)₃CNH | O | O | CONHn-Bu | O | O | 1 | C | valproic acid |
| D-274 | PhO | O | O | CONHn-Bu | O | O | 1 | C | valproic acid |
| D-275 | PhNH | O | O | CONHn-Bu | O | O | 1 | C | valproic acid |
| D-276 | 4Cl(Ph) | O | O | CONHn-Bu | O | O | 1 | C | valproic acid |
| D-277 | 4Cl(Ph)O | O | O | CONH—Bn | O | O | 1 | C | valproic acid |
| D-278 | 4Cl(Ph)NH | O | O | CONH—Bn | O | O | 1 | C | valproic acid |
| D-279 | MeO | O | O | CONH—Bn | O | O | 1 | C | valproic acid |
| D-280 | EtO | O | O | CONNH₂ | O | O | 1 | C | valproic acid |
| D-281 | n-PrO | O | O | CONHNHMe | O | O | 1 | C | valproic acid |
| D-282 | (CH₃)₃CO | O | O | CONHNHEt | O | O | 1 | C | valproic acid |
| D-283 | MeNH | O | O | CONHNHPr | O | O | 1 | C | valproic acid |
| D-284 | EtNH | O | O | CONHNHt-Bu | O | O | 1 | C | valproic acid |
| D-285 | n-PrNH | O | O | CONHNHCO₂Me | O | O | 1 | C | valproic acid |
| D-286 | (CH₃)₃CNH | O | O | CONHNHCO₂Et | O | O | 1 | C | valproic acid |
| D-287 | PhO | O | O | CONHNHCO₂t-Bu | O | O | 1 | C | valproic acid |
| D-288 | PhNH | O | O | CONHNHCO₂Bn | O | O | 1 | C | valproic acid |
| D-289 | 4Cl(Ph) | O | O | CONHN═CHMe | O | O | 1 | C | valproic acid |
| D-290 | 4Cl(Ph)O | O | O | CONHN═CHMe | O | O | 1 | C | valproic acid |
| D-291 | 4Cl(Ph)NH | O | O | CONHN═CHPh | O | O | 1 | C | valproic acid |
| D-292 | MeO | O | O | CONHN═CHPh | O | O | 1 | C | valproic acid |
| D-293 | EtO | O | O | CO₂H | O | O | 1 | C | valproic acid |
| D-294 | n-PrO | O | O | CO₂H | O | O | 1 | C | valproic acid |
| D-295 | (CH₃)₃CO | O | O | CO₂H | O | O | 1 | C | valproic acid |
| D-296 | MeNH | O | O | CO₂H | O | O | 1 | C | valproic acid |
| D-297 | EtNH | O | O | CO₂H | O | O | 1 | C | valproic acid |
| D-298 | n-PrNH | O | O | CO₂H | O | O | 1 | C | valproic acid |
| D-299 | (CH₃)₃CNH | O | O | CO₂H | O | O | 1 | C | valproic acid |
| D-300 | PhO | O | O | CO₂Me | O | O | 1 | C | valproic acid |
| D-301 | PhNH | O | O | CO₂Me | O | O | 1 | C | valproic acid |
| D-302 | 4Cl(Ph) | O | O | CO₂Me | O | O | 1 | C | valproic acid |
| D-303 | 4Cl(Ph)O | O | O | CO₂Me | O | O | 1 | C | valproic acid |
| D-304 | 4CH₃O(Ph)O | O | O | CO₂Me | O | O | 1 | C | valproic acid |
| D-305 | 4NO₂(Ph)O | O | O | CO₂Me | O | O | 1 | C | valproic acid |
| D-306 | 4Cl(Ph)NH | O | O | CO₂Me | O | O | 1 | C | valproic acid |
| D-307 | 4F(Ph)NH | O | O | CO₂Et | O | O | 1 | C | valproic acid |
| D-308 | 4MeO(Ph)NH | O | O | CO₂Et | O | O | 1 | C | valproic acid |
| D-309 | MeO | O | S | CO₂n-Pr | O | O | 1 | C | valproic acid |
| D-310 | EtO | O | S | CO₂n-Pr | O | O | 1 | C | valproic acid |
| D-311 | n-PrO | O | O | CO₂n-Bu | O | O | 1 | C | valproic acid |
| D-312 | (CH₃)₃CO | O | O | CO₂n-Bu | O | O | 1 | C | valproic acid |
| D-313 | MeNH | O | O | CO₂n-Bu | O | O | 1 | C | valproic acid |
| D-314 | EtNH | O | S | CO₂n-Bu | O | O | 1 | C | valproic acid |
| D-315 | n-PrNH | O | S | CO₂n-Bu | O | O | 1 | C | valproic acid |
| D-316 | (CH₃)₃CNH | O | O | CO₂n-Bu | O | O | 1 | C | valproic acid |
| D-317 | PhO | O | O | CO₂n-Bu | O | O | 1 | C | valproic acid |
| D-318 | PhNH | O | O | CO₂n-Bu | O | O | 1 | C | valproic acid |
| D-319 | 4Cl(Ph) | O | O | CO₂n-Bu | O | O | 1 | C | valproic acid |
| D-320 | methyl | O | O | CO₂—Bn | — | — | 0 | N | acepromazine |
| D-321 | ethyl | O | O | CO₂—Bn | — | — | 0 | N | aminopropazine |
| D-322 | n-propyl | O | O | CO₂—Bn | — | — | 0 | N | amiodarone |
| D-323 | C(CH₃)₃ | O | S | CO₂—Bn | — | — | 0 | N | amitriptyline |
| D-324 | MeO | O | S | CO₂—Bn | — | — | 0 | N | atropine |
| D-325 | EtO | O | O | CO₂—Bn | — | — | 0 | N | atropine |
| D-326 | n-PrO | O | O | CO₂—Bn | — | — | 0 | O | atropine |
| D-327 | MeNH | O | O | CO₂—Bn | — | — | 0 | O | atropine |
| D-328 | EtNH | O | O | CO₂—Bn | — | — | 0 | N | azaperone |
| D-329 | n-PrNH | O | O | COSMe | — | — | 0 | N | buspirone |
| D-330 | n-PrO | O | S | COSMe | — | — | 0 | N | chlorpheniramine |
| D-331 | PhO | O | O | COSMe | — | — | 0 | N | clemastine |
| D-332 | Ph—NH | O | O | COSMe | — | — | 0 | N | clomipramine |
| D-333 | 4Cl(Ph) | O | S | COSEt | — | — | 0 | N | cyproheptadine |
| D-334 | 4Cl(Ph)O | O | O | COSEt | — | — | 0 | N | diethylcarbamazine |
| D-335 | 4CH₃O(Ph)O | O | O | COSn-Bu | — | — | 0 | N | diltiazem |
| D-336 | 4NO₂(Ph)O | O | S | COSn-Bu | — | — | 0 | N | diphenhydramine |
| D-337 | 4Cl(Ph)NH | O | O | COSBn | — | — | 0 | N | diphenoxylate |
| D-338 | 4F(Ph)NH | O | O | COSBn | — | — | 0 | N | doxapram |
| D-339 | 4MeO(Ph)NH | O | O | CONHMe | — | — | 0 | N | doxepin |
| D-340 | pyrid-2-yl | O | O | CONHMe | — | — | 0 | N | doxylamine |
| D-341 | pyrid-4-yl | O | O | CONHEt | — | — | 0 | N | droperidol |
| D-342 | MeO | O | O | CONHn-Pr | — | — | 0 | N | fentanyl |
| D-343 | EtO | O | O | CONHn-Bu | — | — | 0 | N | fentanyl |
| D-344 | n-PrO | O | O | CONHn-Bu | — | — | 0 | N | fluconazole |
| D-345 | PhO | O | O | CONHn-Bu | — | — | 0 | N | fluconazole |
| D-346 | MeNH | O | O | CONHn-Bu | — | — | 0 | N | fluconazole |
| D-347 | Ph—NH | O | O | CONHn-Bu | — | — | 0 | N | fluconazole |
| D-348 | EtNH | O | O | CONHn-Bu | — | — | 0 | N | fluconazole |
| D-349 | n-PrNH | O | O | CONHn-Bu | — | — | 0 | N | fluconazole |
| D-350 | (CH₃)₃CNH | O | O | CONH—Bn | — | — | 0 | N | fluconazole |
| D-351 | PhO | O | O | CONH—Bn | — | — | 0 | N | fluconazole |
| D-352 | Ph—NH | O | O | CONH—Bn | — | — | 0 | N | fluconazole |
| D-353 | 4Cl(Ph) | O | O | CONNH₂ | — | — | 0 | N | fluconazole |
| D-354 | methyl | O | O | CONHNHMe | — | — | 0 | N | fluconazole |
| D-355 | ethyl | O | O | CONHNHEt | — | — | 0 | N | fluconazole |
| D-356 | n-propyl | O | O | CONHNHPr | — | — | 0 | N | fluconazole |
| D-357 | C(CH₃)₃ | O | O | CONHNHt-Bu | — | — | 0 | N | fluconazole |
| D-358 | MeO | O | O | CONHNHCO₂Me | — | — | 0 | N | fluconazole |
| D-359 | EtO | O | O | CONHNHCO₂Et | — | — | 0 | N | fluconazole |
| D-360 | n-PrO | O | O | CONHNHCO₂t-Bu | — | — | 0 | N | fluconazole |
| D-361 | MeNH | O | O | CONHNHCO₂Bn | — | — | 0 | N | fluconazole |
| D-362 | EtNH | O | O | CONHN═CHMe | — | — | 0 | N | fluconazole |
| D-363 | n-PrNH | O | O | CONHN═CHMe | — | — | 0 | N | fluconazole |
| D-364 | (CH₃)₃CNH | O | O | CONHN═CHPh | — | — | 0 | N | fluconazole |
| D-365 | PhO | O | O | CONHN═CHPh | — | — | 0 | N | fluconazole |
| D-366 | methyl | O | O | CO₂H | — | — | 0 | N | fluconazole |
| D-367 | ethyl | O | O | CO₂H | — | — | 0 | N | fluconazole |
| D-368 | n-propyl | O | O | CO₂H | — | — | 0 | N | fluconazole |
| D-369 | C(CH₃)₃ | O | O | CO₂H | — | — | 0 | N | fluconazole |
| D-370 | MeO | O | O | CO₂H | — | — | 0 | N | fluconazole |
| D-371 | EtO | O | O | CO₂H | — | — | 0 | N | fluconazole |
| D-372 | n-PrO | O | O | CO₂H | — | — | 0 | N | fluconazole |
| D-373 | MeNH | O | O | CO₂Me | — | — | 0 | N | fluconazole |
| D-374 | EtNH | O | O | CO₂Me | — | — | 0 | N | fluconazole |
| D-375 | n-PrNH | O | O | CO₂Me | — | — | 0 | N | fluconazole |
| D-376 | n-PrO | O | O | CO₂Me | — | — | 0 | N | fluconazole |
| D-377 | PhO | O | O | CO₂Me | — | — | 0 | N | flucanazole |
| D-378 | Ph—NH | O | O | CO₂Me | — | — | 0 | N | fluconazole |
| D-379 | 4Cl(Ph) | O | O | CO₂Me | — | — | 0 | N | fluconazole |
| D-380 | 4Cl(Ph)O | O | O | CO₂Et | — | — | 0 | N | fluconazole |
| D-381 | 4Cl(Ph)NH | O | O | CO₂Et | — | — | 0 | N | fluconazole |
| D-382 | MeO | O | S | CO₂n-Pr | — | — | 0 | N | fluconazole |
| D-383 | EtO | O | S | CO₂n-Pr | — | — | 0 | N | fluconazole |
| D-384 | n-PrO | O | O | CO₂n-Bu | — | — | 0 | N | fluconazole |
| D-385 | MeNH | O | O | CO₂n-Bu | — | — | 0 | N | fluconazole |
| D-386 | EtNH | O | O | CO₂n-Bu | — | — | 0 | N | fluconazole |
| D-387 | n-PrNH | O | S | CO₂n-Bu | — | — | 0 | N | fluconazole |
| D-388 | n-PrO | O | S | CO₂n-Bu | — | — | 0 | O | fluconazole |
| D-389 | PhO | O | O | CO₂n-Bu | — | — | 0 | O | fluconazole |
| D-390 | PhNH | O | O | CO₂n-Bu | — | — | 0 | O | fluconazole |
| D-391 | 4Cl(Ph) | O | O | CO₂n-Bu | — | — | 0 | O | fluconazole |
| D-392 | 4Cl(Ph)O | O | O | CO₂n-Bu | — | — | 0 | O | fluconazole |
| D-393 | MeO | O | O | CO₂—Bn | — | — | 0 | O | fluconazole |
| D-394 | EtO | O | O | CO₂—Bn | — | — | 0 | O | fluconazole |
| D-395 | n-PrO | O | O | CO₂—Bn | — | — | 0 | O | fluconazole |
| D-396 | (CH₃)₃CO | O | S | CO₂—Bn | — | — | 0 | O | fluconazole |
| D-397 | MeNH | O | S | CO₂—Bn | — | — | 0 | O | fluconazole |
| D-398 | EtNH | O | O | CO₂—Bn | — | — | 0 | O | fluconazole |
| D-399 | n-PrNH | O | O | CO₂—Bn | — | — | 0 | O | fluconazole |
| D-400 | (CH₃)₃CNH | O | O | CO₂—Bn | — | — | 0 | O | fluconazole |
| D-401 | PhO | O | O | CO₂—Bn | — | — | 0 | O | fluconazole |
| D-402 | Ph—NH | O | O | COSMe | — | — | 0 | O | fluconazole |
| D-403 | 4Cl(Ph) | O | S | COSMe | — | — | 0 | O | fluconazole |
| D-404 | methyl | O | O | COSMe | — | — | 0 | O | fluconazole |
| D-405 | ethyl | O | O | COSMe | — | — | 0 | O | fluconazole |
| D-406 | n-propyl | O | S | COSEt | — | — | 0 | O | fluconazole |
| D-407 | C(CH₃)₃ | O | O | COSEt | — | — | 0 | O | fluconazole |
| D-408 | MeO | O | O | COSn-Bu | — | — | 0 | O | fluconazole |
| D-409 | EtO | O | S | COSn-Bu | — | — | 0 | O | fluconazole |
| D-410 | n-PrO | O | O | COSBn | — | — | 0 | O | fluconazole |
| D-411 | MeNH | O | O | COSBn | — | — | 0 | O | fluconazole |
| D-412 | EtNH | O | O | CONHMe | — | — | 0 | O | fluconazole |
| D-413 | n-PrNH | O | O | CONHMe | — | — | 0 | O | fluconazole |
| D-414 | n-PrO | O | O | CONHEt | — | — | 0 | O | fluconazole |
| D-415 | PhO | O | O | CONHn-Pr | — | — | 0 | N | hydrocodone |
| D-416 | Ph—NH | O | O | CONHn-Bu | — | — | 0 | O | hydrocodone |
| D-417 | methyl | O | O | CONHn-Bu | — | — | 0 | N | hydroxyzine |
| D-418 | ethyl | O | O | CONHn-Bu | — | — | 0 | N | itraconazole |
| D-419 | n-propyl | O | O | CONHn-Bu | — | — | 0 | N | levamisole |
| D-420 | C(CH₃)₃ | O | O | CONHn-Bu | — | — | 0 | N | meclizine |
| D-421 | MeO | O | O | CONHn-Bu | — | — | 0 | N | meclizine |
| D-422 | EtO | O | O | CONHn-Bu | — | — | 0 | N | meclizine |
| D-423 | n-PrO | O | O | CONH—Bn | — | — | 0 | N | meclizine |
| D-424 | MeNH | O | O | CONH—Bn | — | — | 0 | N | meclizine |
| D-425 | EtNH | O | O | CONH—Bn | — | — | 0 | N | meclizine |
| D-426 | n-PrNH | O | O | CONNH₂ | — | — | 0 | N | meclizine |
| D-427 | n-PrO | O | O | CONHNHMe | — | — | 0 | N | meclizine |
| D-428 | PhO | O | O | CONHNHEt | — | — | 0 | N | meclizine |
| D-429 | Ph—NH | O | O | CONHNHPr | — | — | 0 | N | meclizine |
| D-430 | 4Cl(Ph) | O | O | CONHNHt-Bu | — | — | 0 | N | meclizine |
| D-431 | 4Cl(Ph)O | O | O | CONHNHCO₂Me | — | — | 0 | N | meclizine |
| D-432 | 4Cl(Ph)NH | O | O | CONHNHCO₂Et | — | — | 0 | N | meclizine |
| D-433 | MeO | O | O | CONHNHCO₂t-Bu | — | — | 0 | N | mecizine |
| D-434 | EtO | O | O | CONHNHCO₂Bn | — | — | 0 | N | meclizine |
| D-435 | n-PrO | O | O | CONHN═CHMe | — | — | 0 | N | meclizine |
| D-436 | MeNH | O | O | C0NHN═CHMe | — | — | 0 | N | meclizine |
| D-437 | EtNH | O | O | CONHN═CHPh | — | — | 0 | N | meclizine |
| D-438 | n-PrNH | O | O | CONHN═CHPh | — | — | 0 | N | meclizine |
| D-439 | methyl | O | O | CO₂H | — | — | 0 | N | meperidine |
| D-440 | ethyl | O | O | CO₂H | — | — | 0 | N | meperidine |
| D-441 | n-propyl | O | O | CO₂H | — | — | 0 | N | meperidine |
| D-442 | C(CH₃)₃ | O | O | CO₂H | — | — | 0 | N | meperidine |
| D-443 | MeO | O | O | CO₂H | — | — | 0 | N | meperidine |
| D-444 | EtO | O | O | CO₂H | — | — | 0 | N | meperidine |
| D-445 | n-PrO | O | O | CO₂H | — | — | 0 | N | meperidine |
| D-446 | MeNH | O | O | CO₂Me | — | — | 0 | N | meperidine |
| D-447 | EtNH | O | O | CO₂Me | — | — | 0 | N | meperidine |
| D-448 | n-PrNH | O | O | CO₂Me | — | — | 0 | N | meperidine |
| D-449 | n-PrO | O | O | CO₂Me | — | — | 0 | N | meperidine |
| D-450 | PhO | O | O | CO₂Me | — | — | 0 | N | meperidine |
| D-451 | Ph—NH | O | O | CO₂Me | — | — | 0 | N | meperidine |
| D-452 | 4Cl(Ph) | O | O | CO₂Me | — | — | 0 | N | meperidine |
| D-453 | 4Cl(Ph)O | O | O | CO₂Et | — | — | 0 | O | meperidine |
| D-454 | 4Cl(Ph)NH | O | O | CO₂Et | — | — | 0 | O | meperidine |
| D-455 | methyl | O | O | CO₂n-Pr | — | — | 0 | O | meperidine |
| D-456 | ethyl | O | O | CO₂n-Pr | — | — | 0 | O | meperidine |
| D-457 | n-propyl | O | O | CO₂n-Bu | — | — | 0 | N | meperidine |
| D-458 | C(CH₃)₃ | O | O | CO₂n-Bu | — | — | 0 | N | meperidine |
| D-459 | MeO | O | O | CO₂n-Bu | — | — | 0 | N | meperidine |
| D-460 | EtO | O | S | CO₂n-Bu | — | — | 0 | N | meperidine |
| D-461 | n-PrO | O | S | CO₂n-Bu | — | — | 0 | N | meperidine |
| D-462 | MeNH | O | O | CO₂n-Bu | — | — | 0 | N | meperidine |
| D-463 | EtNH | O | O | CO₂n-Bu | — | — | 0 | N | meperidine |
| D-464 | n-PrNH | O | O | CO₂n-Bu | — | — | 0 | N | meperidine |
| D-465 | n-PrO | O | O | CO₂n-Bu | — | — | 0 | N | meperidine |
| D-466 | PhO | O | O | CO₂—Bn | — | — | 0 | N | meperidine |
| D-467 | Ph—NH | O | O | CO₂—Bn | — | — | 0 | N | meperidine |
| D-468 | 4Cl(Ph) | O | O | CO₂—Bn | — | — | 0 | N | meperidine |
| D-469 | 4Cl(Ph)O | O | S | CO₂—Bn | — | — | 0 | N | meperidine |
| D-470 | 4MeO(Ph)O | O | S | CO₂—Bn | — | — | 0 | N | meperidine |
| D-471 | 4NO₂(Ph)O | O | O | CO₂—Bn | — | — | 0 | N | meperidine |
| D-472 | 4Cl(Ph)NH | O | O | CO₂—Bn | — | — | 0 | N | meperidine |
| D-473 | 4F(Ph)NH | O | O | CO₂—Bn | — | — | 0 | N | meperidine |
| D-474 | 4MeO(Ph)NH | O | O | CO₂—Bn | — | — | 0 | N | meperidine |
| D-475 | pyrid-2-yl | O | O | COSMe | — | — | 0 | N | meperidine |
| D-476 | pyrid-4-yl | O | S | COSMe | — | — | 0 | N | meperidine |
| D-477 | methyl | O | O | COSMe | — | — | 0 | N | meperidine |
| D-478 | ethyl | O | O | COSMe | — | — | 0 | N | meperidine |
| D-479 | methyl | O | S | COSEt | — | — | 0 | N | meperidine |
| D-480 | ethyl | O | O | COSEt | — | — | 0 | N | meperidine |
| D-481 | n-propyl | O | O | COSn-Bu | — | — | 0 | N | meperidine |
| D-482 | C(CH₃)₃ | O | S | COSn-Bu | — | — | 0 | N | meperidine |
| D-483 | MeO | O | O | COSBn | — | — | 0 | N | meperidine |
| D-484 | EtO | O | O | COSBn | — | — | 0 | N | meperidine |
| D-485 | n-PrO | O | O | CONHMe | — | — | 0 | N | meperidine |
| D-486 | MeNH | O | O | CONEMe | — | — | 0 | N | meperidine |
| D-487 | EtNH | O | O | CONHEt | — | — | 0 | N | meperidine |
| D-488 | n-PrNH | O | O | CONHn-Pr | — | — | 0 | N | meperidine |
| D-489 | n-PrO | O | O | CONHn-Bu | — | — | 0 | N | meperidine |
| D-490 | PhO | O | O | CONHn-Bu | — | — | 0 | N | meperidine |
| D-491 | Ph—NH | O | O | CONHn-Bu | — | — | 0 | N | meperidine |
| D-492 | 4Cl(Ph) | O | O | CONHn-Bu | — | — | 0 | N | meperidine |
| D-493 | 4Cl(Ph)O | O | O | CONHn-Bu | — | — | 0 | N | meperidine |
| D-494 | 4CH₃O(Ph)O | O | O | CONHn-Bu | — | — | 0 | N | meperidine |
| D-495 | 4NO₂(Ph)O | O | O | CONHn-Bu | — | — | 0 | N | meperidine |
| D-496 | 4Cl(Ph)NH | O | O | CONH—Bn | — | — | 0 | N | methenamine |
| D-497 | 4F(Ph)NH | O | O | CONH—Bn | — | — | 0 | N | methenamine |
| D-498 | 4MeO(Ph)NH | O | O | CONH—Bn | — | — | 0 | N | methenamine |
| D-499 | pyrid-2-yl | O | O | CONNH₂ | — | — | 0 | N | methenamine |
| D-500 | pyrid-4-yl | O | O | CONHNHMe | — | — | 0 | N | methenamine |
| D-501 | methyl | O | O | CONHNHEt | — | — | 0 | N | methenamine |
| D-502 | ethyl | O | O | CONHNHPr | — | — | 0 | N | methenamine |
| D-503 | n-propyl | O | O | CONHNHt-Bu | — | — | 0 | N | methenamine |
| D-504 | C(CH₃)₃ | O | O | CONHNHCO₂Me | — | — | 0 | N | methenamine |
| D-505 | MeO | O | O | CONHNHCO₂Et | — | — | 0 | N | methenamine |
| D-506 | EtO | O | O | CONHNHCO₂t-Bu | — | — | 0 | N | methenamine |
| D-507 | n-PrO | O | O | CONHNHCO₂Bn | — | — | 0 | N | methenamine |
| D-508 | MeNH | O | O | CONHN═CHMe | — | — | 0 | N | methenamine |
| D-509 | EtNH | O | O | CONHN═CHMe | — | — | 0 | N | methenamine |
| D-510 | n-PrNH | O | O | CONHN═CHPh | — | — | 0 | N | methenamine |
| D-511 | n-PrO | O | O | CONHN═CHPh | — | — | 0 | N | methenamine |
| D-512 | PhO | O | O | CO₂H | — | — | 0 | N | methenamine |
| D-513 | Ph—NH | O | O | CO₂H | — | — | 0 | N | morantel |
| D-514 | 4Cl(Ph) | O | O | CO₂H | — | — | 0 | N | morantel |
| D-515 | 4Cl(Ph)O | O | O | CO₂H | — | — | 0 | N | morantel |
| D-516 | 4CH₃O(Ph)O | O | O | CO₂H | — | — | 0 | N | morantel |
| D-517 | 4NO₂(Ph)O | O | O | CO₂H | — | — | 0 | N | morantel |
| D-518 | 4Cl(Ph)NH | O | O | CO₂H | — | — | 0 | N | morantel |
| D-519 | 4F(Ph)NH | O | O | CO₂Me | — | — | 0 | N | morantel |
| D-520 | 4MeO(Ph)NH | O | O | CO₂Me | — | — | 0 | N | morantel |
| D-521 | pyrid-2-yl | O | O | CO₂Me | — | — | 0 | N | morantel |
| D-522 | pyrid-4-yl | O | O | CO₂Me | — | — | 0 | N | morantel |
| D-523 | methyl | O | O | CO₂Me | — | — | 0 | N | morantel |
| D-524 | ethyl | O | O | CO₂Me | — | — | 0 | N | morantel |
| D-525 | n-propyl | O | O | CO₂Me | — | — | 0 | N | morantel |
| D-526 | C(CH₃)₃ | O | O | CO₂Et | — | — | 0 | N | morantel |
| D-527 | MeO | O | O | CO₂Et | — | — | 0 | N | morantel |
| D-528 | EtO | O | O | CO₂n-Pr | — | — | 0 | N | morantel |
| D-529 | n-PrO | O | O | CO₂n-Pr | — | — | 0 | N | morantel |
| D-530 | MeNH | O | O | CO₂n-Bu | — | — | 0 | N | morantel |
| D-531 | EtNH | O | O | CO₂n-Bu | — | — | 0 | N | morantel |
| D-532 | n-PrNH | O | O | CO₂n-Bu | — | — | 0 | N | morantel |
| D-533 | n-PrO | O | O | CO₂n-Bu | — | — | 0 | N | naltrexone |
| D-534 | PhO | O | O | CO₂n-Bu | — | — | 0 | N | naltrexone |
| D-535 | Ph—NH | O | S | CO₂n-Bu | — | — | 0 | N | naltrexone |
| D-536 | 4Cl(Ph) | O | S | CO₂n-Bu | — | — | 0 | N | naltrexone |
| D-537 | 4Cl(Ph)O | O | S | CO₂n-Bu | — | — | 0 | N | naltrexone |
| D-538 | 4CH₃O(Ph)O | O | O | CO₂n-Bu | — | — | 0 | N | naltrexone |
| D-539 | 4NO₂(Ph)O | O | O | CO₂—Bn | — | — | 0 | N | naltrexone |
| D-540 | 4Cl(Ph)NH | O | O | CO₂—Bn | — | — | 0 | N | naltrexone |
| D-541 | 4F(Ph)NH | O | O | CO₂—Bn | — | — | 0 | N | naltrexone |
| D-542 | 4MeO(Ph)NH | O | O | CO₂—Bn | — | — | 0 | N | naltrexone |
| D-543 | pyrid-2-yl | O | O | CO₂—Bn | — | — | 0 | N | naltrexone |
| D-544 | pyrid-4-yl | O | O | CO₂—Bn | — | — | 0 | N | naltrexone |
| D-545 | methyl | O | O | CO₂—Bn | — | — | 0 | N | oxybutynin |
| D-546 | ethyl | O | O | CO₂—Bn | — | — | 0 | N | oxybutynin |
| D-547 | n-propyl | O | O | CO₂—Bn | — | — | 0 | N | oxybutynin |
| D-548 | C(CH₃)₃ | O | O | COSMe | — | — | 0 | N | oxybutynin |
| D-549 | MeO | O | O | COSMe | — | — | 0 | N | oxybutynin |
| D-550 | EtO | O | O | COSMe | — | — | 0 | N | oxybutynin |
| D-551 | n-PrO | O | O | COSMe | — | — | 0 | N | oxybutynin |
| D-552 | MeNH | O | O | COSEt | — | — | 0 | N | oxybutynin |
| D-553 | EtNH | O | O | COSEt | — | — | 0 | N | pentazocine |
| D-554 | n-PrNH | O | O | COSn-Bu | — | — | 0 | N | pentazocine |
| D-555 | n-PrO | O | O | COSn-Bu | — | — | 0 | N | pentazocine |
| D-556 | PhO | O | O | COSBn | — | — | 0 | N | pentazocine |
| D-557 | Ph—NH | O | O | COSBn | — | — | 0 | N | pentazocine |
| D-558 | 4Cl(Ph) | O | O | CONHMe | — | — | 0 | N | pentazocine |
| D-559 | 4Cl(Ph)O | O | O | CONHMe | — | — | 0 | N | pentazocine |
| D-560 | 4MeO(Ph)O | O | O | CONHEt | — | — | 0 | N | pentazocine |
| D-561 | methyl | O | O | CONHn-Pr | — | — | 0 | N | pentazocine |
| D-562 | ethyl | O | O | CONHn-Bu | — | — | 0 | N | pentazocine |
| D-563 | n-propyl | O | O | CONHn-Bu | — | — | 0 | N | pentazocine |
| D-564 | C(CH₃)₃ | O | O | CONHn-Bu | — | — | 0 | N | pentazocine |
| D-565 | MeO | O | O | CONHn-Bu | — | — | 0 | N | pentazocine |
| D-566 | EtO | O | O | CONHn-Bu | — | — | 0 | N | pentazocine |
| D-567 | n-PrO | O | O | CONHn-Bu | — | — | 0 | N | pentazocine |
| D-568 | MeNH | O | O | CONHn-Bu | — | — | 0 | N | pentazocine |
| D-569 | EtNH | O | O | CONH—Bn | — | — | 0 | N | pentazocine |
| D-570 | n-PrNH | O | O | CONH—Bn | — | — | 0 | N | pentazocine |
| D-571 | n-PrO | O | O | CONH—Bn | — | — | 0 | N | pentazocine |
| D-572 | PhO | O | O | CONNH₂ | — | — | 0 | N | pentazocine |
| D-573 | Ph—NH | O | O | CONHNHMe | — | — | 0 | N | pentazocine |
| D-574 | 4Cl(Ph) | O | O | CONHNHEt | — | — | 0 | N | pentazocine |
| D-575 | 4Cl(Ph)O | O | O | CONHNHPr | — | — | 0 | N | pentazocine |
| D-576 | 4CH₃O(Ph)O | O | O | CONHNHt-Bu | — | — | 0 | N | pentazocine |
| D-577 | 4NO₂(Ph)O | O | S | CONHNHCO₂Me | — | — | 0 | N | pentazocine |
| D-578 | 4Cl(Ph)NH | O | S | CONHNHCO₂Et | — | — | 0 | N | pentazocine |
| D-579 | 4F(Ph)NH | O | S | CONHNHCO₂t-Bu | — | — | 0 | N | pentazocine |
| D-580 | 4MeO(Ph)NH | O | O | CONHNHCO₂Bn | — | — | 0 | N | pentazocine |
| D-581 | pyrid-2-yl | O | O | CONHN═CHMe | — | — | 0 | N | pentazocine |
| D-582 | pyrid-4-yl | O | O | CONHN═CHMe | — | — | 0 | N | pentazocine |
| D-583 | methyl | O | O | CO₂H | — | — | 0 | N | pentazocine |
| D-584 | ethyl | O | O | CO₂H | — | — | 0 | N | promazine |
| D-585 | n-propyl | O | O | CO₂H | — | — | 0 | N | pyrantel |
| D-586 | C(CH₃)₃ | O | O | CO₂H | — | — | 0 | N | selegiline |
| D-587 | MeO | O | O | CO₂H | — | — | 0 | N | tiamulin |
| D-588 | EtO | O | O | CO₂H | — | — | 0 | N | verapamil |
| D-589 | n-PrO | O | O | CO₂H | — | — | 0 | N | verapamil |
| D-590 | MeNH | O | O | CO₂Me | — | — | 0 | N | verapamil |
| D-591 | EtNH | O | O | CO₂Me | — | — | 0 | N | verapamil |
| D-592 | n-PrNH | O | O | CO₂Me | — | — | 0 | N | verapamil |
| D-593 | n-PrO | O | O | CO₂Me | — | — | 0 | N | verapamil |
| D-594 | PhO | O | O | CO₂Me | — | — | 0 | N | verapamil |
| D-595 | Ph—NH | O | O | CO₂Me | — | — | 0 | N | verapamil |
| D-596 | 4Cl(Ph) | O | O | CO₂Me | — | — | 0 | N | verapamil |
| D-597 | 4Cl(Ph)O | O | O | CO₂Et | — | — | 0 | N | verapamil |
| D-598 | 4CH₃O(Ph)O | O | O | CO₂Et | — | — | 0 | N | verapamil |
| D-599 | 4NO₂(Ph)O | O | O | CO₂n-Pr | — | — | 0 | N | verapamil |
| D-600 | 4Cl(Ph)NH | O | O | CO₂n-Pr | — | — | 0 | N | verapamil |
| D-601 | 4F(Ph)NH | O | O | CO₂n-Bu | — | — | 0 | N | verapamil |
| D-602 | 4MeO(Ph)NH | O | O | CO₂n-Bu | — | — | 0 | N | verapamil |
| D-603 | pyrid-2-yl | O | O | CO₂n-Bu | — | — | 0 | N | verapamil |
| D-604 | pyrid-4-yl | O | O | CO₂n-Bu | — | — | 0 | N | verapamil |
| D-605 | 4NO₂(Ph)O | O | O | CO₂n-Bu | — | — | 0 | N | verapamil |
| D-606 | 4Cl(Ph)NH | O | O | CO₂n-Bu | — | — | 0 | N | verapamil |
| D-610 | pyrid-4-yl | O | O | CO₂—Bn | — | — | 0 | N | verapamil |
| D-611 | methyl | O | O | CO₂—Bn | — | — | 0 | N | verapamil |
| D-612 | ethyl | O | O | CO₂—Bn | — | — | 0 | N | verapamil |
| D-613 | n-propyl | O | O | CO₂—Bn | — | — | 0 | N | verapamil |
| D-614 | C(CH₃)₃ | O | O | CO₂—Bn | — | — | 0 | N | verapamil |
| D-615 | MeO | O | S | CO₂—Bn | — | — | 0 | N | verapamil |
| D-616 | EtO | O | S | CO₂—Bn | — | — | 0 | N | verapamil |
| D-617 | n-PrO | O | S | CO₂—Bn | — | — | 0 | N | verapamil |
| D-618 | MeNH | O | O | CO₂—Bn | — | — | 0 | N | verapamil |
| D-619 | EtNH | O | O | COSMe | — | — | 0 | N | verapamil |
| D-620 | n-PrNH | O | O | COSMe | — | — | 0 | N | verapamil |
| D-621 | n-PrO | O | O | COSMe | — | — | 0 | N | verapamil |
| D-622 | PhO | O | O | COSMe | — | — | 0 | N | verapamil |
| D-623 | Ph—NH | O | O | COSEt | — | — | 0 | N | verapamil |
| D-624 | 4Cl(Ph) | O | O | COSEt | — | — | 0 | N | verapamil |
| D-625 | 4Cl(Ph)O | O | O | COSn-Bu | — | — | 0 | N | verapamil |
| D-626 | 4CH₃O(Ph)O | O | O | COSn-Bu | — | — | 0 | N | verapamil |
| D-627 | 4NO₂(Ph)O | O | O | COSBn | — | — | 0 | N | verapamil |
| D-628 | 4Cl(Ph)NH | O | O | COSBn | — | — | 0 | N | verapamil |
| D-629 | 4F(Ph)NH | O | O | CONHMe | — | — | 0 | N | verapamil |
| D-630 | 4MeO(Ph)NH | O | O | CONHMe | — | — | 0 | N | verapamil |
| D-631 | pyrid-2-yl | O | O | CONHEt | — | — | 0 | N | verapamil |
| D-632 | pyrid-4-yl | O | O | CONHn-Pr | — | — | 0 | N | verapamil |

### Escherichia coli 8739 and Staphylococcus aureus 6538 Minimum Inhibitory Concentration (MIC) Test Methods and Results

Several compounds of this invention were tested for biological activity in vitro against Escherichia coli 8739 and Staphylococcus aureus 6538 described below. The following test procedures were employed.

MIC Determination: The lowest concentration of test compound required to inhibit Escherichia coli 8739 and Staphylococcus aureus 6538 was determined by a high resolution minimum inhibitory concentration (HRMIC) test. Varying amounts of the test compounds were added to minimal salts glucose medium (Maniatis, T., Fritsch, E. F., Sambrook, J. 1982 Molecular Cloning, page 68) supplemented with 0.1% yeast extract (M9GY) in a 96-well microtiter plate. Ten-fold serial dilutions were performed on a Biomek 2000 Workstation to obtain a range of closely spaced concentrations of test compound as shown in FIG. 1. A cell suspension of inoculum, adjusted to provide 10⁶ CFU/mL in each well, was added to the microtiter plate. Microtiter plates were incubated at 30° C. for 24 h and then were checked for the presence or absence of microbial growth in each well. The concentration of compound in the first microtiter well demonstrating no growth was the minimum inhibitory concentration (MIC) for the test compound.

[image]

TABLE 11
MIC (ppm AI*) of Kathon CG Biocide, Norfloxacin and compound
8-1 vs S. aureus 6538 and E. coli 8739:

| | S. aureus 6538 | E. coli 8739 |
|---|---|---|
| Compound | Gram-positive | Gram-negative |
| Kathon CG Biocide | 0.4 | 0.75 |
| Norfloxacin | 0.9 | 0.2 |
| 8-1 | 0.06 | 2.5 |

TABLE 12
MIC (ppm AI*) of compounds 8-2, 8-3 and 8-4 vs S. aureus 6538 and
E. coli 8739:

| | S. aureus 6538 | E. coli 8739 |
|---|---|---|
| Compound | Gram-positive | Gram-negative |
| 8-2 | 0.45 | 3 |
| 8-3 | 4.5 | 40 |
| 8-4 | 0.7 | >1000 |

We claim:

1. A pharmaceutical moiety represented by Z¹(X¹), wherein
X¹ is an oxygen atom, a sulfur atom, a phosphorous atom or a nitrogen atom attached to Z¹,
Z¹ represents the remainder of said pharmaceutical moiety,
m is 1, and
Z¹(X¹)ₘ—H represents the complete pharmaceutical compound, said pharmaceutical moiety Z¹(X¹)ₘ being substituted with a second moiety on X¹, said second moiety comprising a substituent which enhances or changes the properties of said pharmaceutical compound, said substituent having the formula 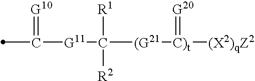
wherein
G¹⁰, G¹¹ and G²⁰ are each independently an oxygen atom or a sulfur atom,
G²¹ is an oxygen atom, a sulfur atom or NR³,
q is 0,
t is 0 or 1,
●- represents the connection point of said substituent to said pharmaceutical moiety Z¹(X¹)ₘ,
Z²(X²)_{q} is a hydrogen atom, alkyl, alkylcarbonyloxyalkyl, alkylcarbonyl, alkenylcarbonyl, alkynylcarbonyl, hydroxyalkyl, alkylsulfonylalkyl, alkylcarbonylaminoalkyl, arylcarbonylaminoalkyl, heteroarylcarbonylaminoalkyl, haloalkyl, alkenyl, acetylaminoalkenyl, alkylcarbonylaminoalkenyl, arylcarbonylaminoalkenyl, heteroarylcarbonylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkyltlioaalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and alkylNR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, alkylcarbonylalkyl, alkenylcarbonylalkyl, alkynylcarbonylalkyl, beterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl, or heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(—N-G²²)R² when q is 0 and t is 1,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M⁻}, OR³, S(O)ⱼR³ or SO₂NR³R⁴ when both q and t are 0 wherein M is halo, hydroxy, alkoxy or the anion of a carboxylic acid and j is 0, 1 or 2,
R¹ is 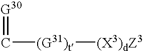
wherein
G³⁰ is an oxygen atom or a sulfur atom,
G³¹ is an oxygen atom, a sulfur atom or NR³,
d is 0,
t′ is 0 or 1,
Z³(X³)_{d}, when d is 0 and t′ is 1, is a hydrogen atom, alkyl, alkylcarbonyloxyalkyl, alkylcarbonyl, alkylcarbonylalkyl, alkenylcarbonyl, alkenylcarbonylalkyl, alkynylcarbonyl, alkynylcarbonylalkyl, hydroxyalkyl, alkylsulfonylalkyl, alkylcarbonylaminoalkyl, arylcarbonylaminoalkyl, heteroarylcarbonylalkylamino, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloaLkenylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, arylcarbonyloxyalkyl, arylcarbonylalkyl, aroxycarbonylalkyl, or aryl, arylcarbonyloxyalkyl, arylcarbonylalkyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkylcarbonyloxyalkyl, aralkylcarbonylalkyl, aralkoxycarbonylalkyl, aralkenyl, aralkynyl, arcycloalkyl, aroxyalkyl, or aralkyl, aralkylcarbonyloxyalkyl, aralkylcarbonylalkyl, aralkoxycarbonylalkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyloxyalkyl, heteroarylcarbonylalkyl, heteroaroxycarbonylalkyl, or heteroaryl, heteroarylcarbonyloxyalkyl, heteroarylarylcarbonylalkyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl, or heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl substituted with one or more substituents independently selected from halo, hydroxy, cyano, nitro, alkyl, alkenyl, alkynyl, haloalkyl, haloalkenyl, haloalkynyl, aLkoxy, haloalkoxy, SO₂NR³R⁴ and NR³R⁴ wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyciylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl, aroxycarbonylalkyl, or arylcarbonyl, aralkylcarbonyl, aralkenylcarbonyl, aralkynylcarbonyl, aroxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl, or heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring, or
the pharmaceutically acceptable salts, isomers, tautomers, enantiomers and mixtures thereof.

2. A pharmaceutical moiety represented by Z¹(X¹)ₘ wherein
X¹ is an oxygen atom, a sulfur atom, a phosphorous atom or a nitrogen atom attached to Z¹,
Z¹ represents the remainder of said pharmaceutical moiety,
m is 1, and
Z¹(X¹)ₘ—H represents the complete pharmaceutical compound,
said pharmaceutical moiety Z¹(X¹)ₘ being substituted with a second moiety on X¹, said second moiety comprising a substituent which enhances or changes the properties of said pharmaceutical compound, said substituent having the formula 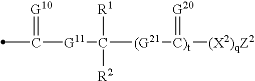
wherein
G¹⁰, G¹¹ and G²⁰ are each independently an oxygen atom or a sulfur atom,
G²¹ is an oxygen atom, a sulfur atom or NR³,
q is 0 or 1,
●- represents the connection point of said substituent to said pharmaceutical moiety Z¹(X¹)ₘ,
X² is an oxygen atom, a sulfur atom, a phosphorous atom, a nitrogen atom or a carbon atom attached to Z²,
t is 0 or 1,
Z²(X²)_{q}(C(=G²⁰)G²¹)ₜ is a second pharmaceutical moiety when q is 1 wherein Z²(X²)_{q}(C(=G²⁰)G²¹)ₜ or Z²(X²)_{q}(C(=G²⁰)G²′)ₜ—H represents the second pharmaceutical,
Z² represents the remainder of said second pharmaceutical moiety,
R¹ is 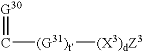
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³′ is an oxygen atom or a sulfur atom,
Z³(X³)_{d}(G³¹)_{t′} is a second alternative pharmaceutical moiety when d is 1 wherein Z³(X³)_{d}(G³′)ₜ—H represents the second alternative pharmaceutical,
Z³ represents the remainder of said second alternative pharmaceutical moiety,
Z³(X³)_{d}, when d is 0 and t′ is 1, is a hydrogen atom, alkyl, alkylcarbonyloxyalkyl, alkylcarbonyl, alkenylcarbonyl, alkynylcarbonyl, hydroxyalkyl, alkylsulfonylalkyl, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴ wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
R² is a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl, aroxycarbonylalkyl, or arylcarbonyl, aralkylcarbonyl, aralkenylcarbonyl, aralkynylcarbonyl, aroxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroarylcarbonyl, heteroaralkylcarbonyl, heteroaralkenylcarbonyl, heteroaralkynylcarbonyl, heteroaroxyearbonylalkyl, heterocyclylcarboryl, heterocyclyloxycarbonylalkyl, or heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, alkyl, alkenyl, alkynyl, haloalkyl, haloalkenyl, haloalkynyl, alkoxy and haloalkoxy, or R³ and R⁴ taken together with the nitrogen atom to which they are attached form a 5- or 6-membered saturated or unsaturated heterocyclic ring,
(q+d) is 1, or
the pharmaceutically acceptable salts, isomers, tautomers, enantiomers and mixtures thereof.

3. A pharmaceutical compound of the formula 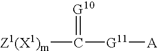
wherein
A is 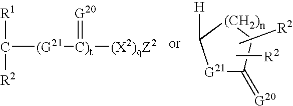
G¹⁰, G¹¹ and G²⁰ are each independently an oxygen atom or a sulfur atom,
G²¹ is an oxygen atom, a sulfur atom or NR³,
X¹ is an oxygen atom, a sulfur atom, a phosphorous atom or a nitrogen atom attached to Z¹,
X² is an oxygen atom, a sulfur atom, a phosphorous atom, a nitrogen atom or a carbon atom attached to Z²,
m, q and t are each independently 0 or 1,
n is 1 or 2,
Z¹(X¹)ₘ is a pharmaceutical moiety when m is 1 wherein Z¹(X¹)-H represents the pharmaceutical,
Z²(X²)_{q}(C(=G²⁰)G²¹)ₜ is a pharmaceutical moiety when q is 1 wherein Z²(X²)_{q}(C(=G²⁰)G²′)ₜ—H or Z²(X²)_{q}(C(=G²⁰)G²′)ₜ represents the pharmaceutical,
Z¹(X¹)ₘ, when m is 0, is a hydrogen atom, halo, alkyl, alkylcarbonyloxyalkyl, alkylcarbonyl, hydroxyalkyl, alkylsulfonylalkyl, acetylaminoalkyl, alkylcarbonylaminoalkyl, arylcarbonylaminoalkyl, heteroarylcarbonylaminoalkyl, haloalkyl, alkenyl, acetylaminoalkenyl, alkylcarbonylaminoalkenyl, arylcarbonylaminoalkenyl, heteroarylcarbonylaminoalkenyl, haloalkenyl, alkynyl, haloaLkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkenyl, alkoxyalkynyl, alkoxycarbonyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, OR³, S(O)ⱼR³, carboxyalkyl, carboxyalkenyl, carboxyalkynyl, aryl, arylcarbonyl, arylcarbonylalkyl, aroxycarbonyl, aroxycarbonylalkyl, or aryl, arylcarbonyl, arylcarbonylalkyl, aroxycarbonyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkenyl, aralkynyl, arcycloalkyl, aroxyalkyl, aralkylcarbonyl, aralkenylcarbonyl, aralkylcarbonylalkyl, aralkenylcarbonylalkyl, or aralkyl, aralkenyl, aralkynyl, arcycloalkyl, aroxyalkyl, aralkylcarbonyl, aralkenylcarbonyl, aralkylcarbonylalkyl, aralkenylcarbonylalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyl, heteroarylcarbonylalkyl, heteroaroxycarbonyl, heteroaroxycarbonylalkyl, or heteroaryl, heteroarylcarbonyl, heteroarylcarbonylalkyl, heteroaroxycarbonyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, heteroaralkylcarbonyl, heteroaralkylcarbonylalkyl, heteroaralkenylcarbonyl, heteroaralkenylcarbonylalkyl, or heteroaralkyl, heteroaralkenyl, heteroaralkynyl, heteroaralkylcarbonyl, heteroaralkylcarbonylalkyl, heteroaralkenylcarbonyl, heteroaralkenylcarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylalkyl, heterocyclylalkenyl, heterocyclylalkynyl, heterocyclylcarbonyl, heterocyclylcarbonylalkyl, heterocyclyloxycarbonyl, heterocyclyloxycarbonylalkyl, or heterocyclyl, heterocyclylalkyl, heterocyclylalkenyl, heterocyclylalkynyl, heterocyclylcarbonyl, heterocyclylcarbonylalkyl, heterocyclyloxycarbonyl, heterocyclyloxycarbonylalkyl substituted with one or more substituents independently selected from halo, hydroxy, cyano, nitro, alkyl, alkenyl, alkvnyl, haloalkyl, haloalkenyl, haloalkynyl, alkoxy, haloalkoxy, SO₂NR³R⁴ and NR³R⁴ wherein j is 0, 1 or 2,
Z²(X²)_{q} is a hydrogen atom, alkyl, alkylcarbonyloxyalkyl, alkylcarbonyl, alkenylcarbonyl, alkynylcarbonyl, hydroxyalkyl, alkylsulfonylalkyl, alkylcarbonylaminoalkyl, arylcarbonylaminoalkyl, heteroarylcarbonylaminoalkyl, haloalkyl, alkenyl, acetylaminoalkenyl, alkylcarbonylaminoalkenyl, arylcarbonylaminoalkenyl, heteroarylcarbonylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, heterocyclyl, heterocyclylalkyl, heterocyclylalkenyl, heterocyclylalkynyl, alkoxyalkyl, alkoxyalkoxyalkyl, alkoxyalkenyl, alkoxyalkynyl, alkoxycarbonyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, NR³R⁴, SO₂NR³R⁴, carboxyalkyl, carboxyalkenyl, carboxyalkynyl, dialkoxyphosphorylalkyl, aryl, aryl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and alkylNR³R⁴, aralkyl, aralkenyl, aralkynyl, arcycloalkyl, aroxyalkyl, or aralkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroaryl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, nitro, cyano, alkyl, alkenyl, alkynyl, haloalkyl, haloalkenyl, haloalkynyl, alkoxy, haloalkoxy, Sv2NR³R⁴ and NR³R⁴, alkylcarbonylalkyl, alkenylcarbonylalkyl, alkynylcarbonylalkyl, heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl, or heterocyclylcarbonyl, heterocyclylcarbonylalkyl, heterocyclyloxycarbonyl, heterocyclyloxycarbonylalkyl, arylcarbonyl, arylcarbonylalkyl, aralkylcarbonyl, aralkylcarbonylalkyl, aroxycarbonyl, aroxycarbonylalkyl, aralkoxycarbonyl, aralkoxycarbonylalkyl, heteroarylcarbonyl, heteroarylcarbonylalkyl, heteroaroxycarbonyl, heteroaroxycarbonylalkyl substituted with one or more substituents independently selected from halo, hydroxy, nitro, cyano, alkyl, alkenyl, alkynyl, haloalkyl, haloalkenyl, haloalkynyl, alkoxy, haloalkoxy, SO₂NR³R⁴ and NR³R⁴, and C(═N-G²²)R² when q is 0 and t is 1,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M-}, OR³, S(O)ⱼR³ or SO₂NR³R⁴ when both q and t are 0 wherein M is halo, hydroxy, alkoxy or the anion of a carboxylic acid and j is 0, 1 or 2,
R¹ is 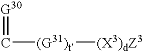
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³¹)ₜ, is a pharmaceutical moiety when d is 1 wherein Z³(X³)_{d}(G³′)ₜ—H represents the pharmaceutical,
Z³(X³)_{d}, when d is 0 and t′ is 1, is a hydrogen atom, alkyl, alkylcarbonyloxyalkyl, alkylcarbonyl, alkylcarbonylalkyl, alkenylcarbonyl, alkenylcarbonylalkyl, alkynylcarbonyl, alkynylcarbonylalkyl, hydroxyalkyl, alkylsulfonylalkyl, alkylcarbonylaminoalkyl, arylcarbonylaminoalkyl, heteroarylcarbonylalkylamino, acetylaminoalkyl, haloalkyl, alkenyl, acetylaminoalkenyl, haloalkenyl, alkynyl, haloalkynyl, cycloalkyl, cycloalkenyl, carboxycycloalkyl, carboxycycloalkenyl, cycloalkylalkyl, cycloalkylalkenyl, cycloalkenylalkyl, cycloalkenylalkenyl, cycloalkylalkynyl, cycloalkenylalkynyl, carboxycycloalkylalkyl, carboxycycloalkylalkenyl, carboxycycloalkenylalkyl, carboxycycloalkenylalkenyl, carboxycycloalkylalkynyl, carboxycycloalkenylalkynyl, alkoxyalkyl, alkoxyalkoxyalkyl, alkoxyalkenyl, alkoxyalkynyl, alkoxycarbonylalkyl, alkoxycarbonylalkenyl, alkoxycarbonylalkynyl, haloalkoxyalkyl, haloalkoxyalkenyl, haloalkoxyalkynyl, alkylthioalkyl, alkylthioalkenyl, alkylthioalkynyl, haloalkylthioalkyl, haloalkylthioalkenyl, haloalkylthioalkynyl, carboxyalkyl, carboxyalkenyl, carboxyalkynyl, NR³R⁴, OR³, S(O)ⱼR³, aryl, arylcarbonyloxyalkyl, arylcarbonylalkyl, aroxycarbonylalkyl, or aryl, arylcarbonyloxyalkyl, arylcarbonylalkyl, aroxycarbonylalkyl substituted with one or more substituents independently selected from halo, nitro, hydroxy, cyano, thiocyanato, alkyl, alkylsulfonylalkyl, alkenyl, alkynyl, haloalkyl, haloalkenyl, haloalkynyl, alkoxy, haloalkoxy, SO₂NR³R⁴ and NR³R⁴, aralkyl, aralkylcarbonyloxyalkyl, aralkylcarbonylalkyl, aralkoxycarbonylalkyl, aralkenyl, aralkynyl, arcycloalkyl, aroxyalkyl, or aralkyl, aralkylcarbonyloxyalkyl, aralkylcarbonylalkyl, aralkoxycarbonylalkyl, aralkenyl, aralkynyl, arcycloalkyl, aroxyalkyl substituted with one or more substituents independently selected from halo, nitro, hydroxy, cyano, alkyl, cycloalkyl, alkenyl, alkynyl, haloalkyl, haloalkenyl, haloalkynyl, alkoxy, haloalkoxy, SO₂NR³R⁴ and NR³R⁴, heteroaryl, heteroarylcarbonyloxyalkyl, heteroarylcarbonylalkyl, heteroaroxycarbonylalkyl, or heteroaryl, heteroarylcarbonyloxyalkyl, heteroarylarylcarbonylalkyl, heteroaroxycarbonylalkyl substituted with one or more substituents independently selected from halo, nitro, hydroxy, cyano, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl, or heteroaralkyl, heteroaralkenyl, heteroaralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl, or heterocyclyl, heterocyclylcarbonyloxyalkyl, heterocyclylcarbonylalkyl, heterocyclyloxycarbonylalkyl, heterocyclylalkyl, heterocyclylalkenyl, heterocyclylalkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴ wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
each R² is independently a hydrogen atom, alkyl, alkenyl, alkynyl, alkoxyalkyl, alkoxyalkenyl, alkoxyalkynyl, alkylthioalkyl, alkylthioalkenyl, alkylthioalkynyl, carboxy, a carboxylate salt, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl, or alkyl, alkenyl, alkynyl, alkoxyalkyl, alkoxyalkenyl, alkoxyalkynyl, alkylthioalkyl, alkylthioalkenyl, alkylthioalkynyl, carboxyalkyl, carboxyalkenyl, carboxyalkynyl, alkoxycarbonyl, alkoxycarbonylalkyl, alkoxycarbonylalkenyl, alkoxycarbonylalkynyl, alkylcarbonyl, alkenylcarbonyl, alkynylcarbonyl, cycloalkyl, cycloalkenyl, cycloalkylalkyl, cycloalkenylalkyl, cycloalkylalkenyl, cycloalkenylalkenyl, cycloalkylalkynyl, cycloalkenylalkynyl, heterocyclyl, heterocyclylalkyl, heterocyclylalkenyl, heterocyclylalkynyl substituted with one or more substituents independently selected from halo, cyano, hydroxy, nitro, SO₂NR³R⁴ and NR³R⁴, aryl or aryl substituted with one or more substituents independently selected from halo, hydroxy, cyano, nitro, alkyl, alkenyl, alkynyl, haloalkyl, haloalkenyl, haloalkynyl, alkoxy, haloalkoxy, carboxy, alkoxycarbonyl, SO₂NR³R⁴ and NR³R⁴, aralkyl, aralkenyl, aralkynyl or aralkyl, aralkenyl, aralkynyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, arylcarbonyl, aralkylcarbonyl, aralkenylcarbonyl, aralkynylcarbonyl, aroxycarbolnylalkyl, or arylcarbonyl, aralkylcarbonyl, aralkenylcarbonyl, aralkynylcarbonyl, aroxycarbonylalkyl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaryl or heteroaryl substituted with one or more substituents independently selected from halo, hydroxy, cyano, nitro, alkyl, alkenyl, alkynyl, haloalkyl, haloalkenyl, haloalkynyl, alkoxy, haloalkoxy, SO₂NR³R⁴ and NR³R⁴, heteroaralkyl, heteroaralkenyl, heteroaralkynyl or heteroaralkyl, heteroaralkenyl, heteroaralkynyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴, heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl, or heteroarylcarbonyl, heteroaralkylcarbonyl, heteroaralkenylcarbonyl, heteroaralkynylcarbonyl, heteroaroxycarbonylalkyl, heterocyclylcarbonyl, heterocyclyloxycarbonylalkyl substituted with one or more substituents independently selected from halo, cyano, hydroxy, nitro, alkyl, alkenyl, alkynyl, haloalkyl, haloalkenyl, haloalkynyl, alkoxy, haloalkoxy, SO₂NR³R⁴ and NR³R⁴,
R³, R⁴ and R⁵ are each independently a hydrogen atom, alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl, alkynyl, or alkyl, cycloalkyl, cycloalkenyl, cycloalkylalkyl, cycloalkylalkenyl, cycloalkylalkynyl, cycloalkenylalkyl, cycloalkenylalkenyl, cycloalkenylalkynyl, carboxyalkyl, carboxyalkenyl, carboxyalkynyl, alkoxyalkyl, alkenyl or alkynyl substituted with one or more halo, aryl, aralkyl, aralkenyl, aralkynyl, or aryl, aralkyl, aralkenyl, aralkynyl substituted with one or more substituents independently selected from halo, alkyl, alkenyl, alkynyl, haloalkyl, haloalkenyl, haloalkynyl, alkoxy and haloalkoxy, heteroaryl, heteroaralkyl, heteroaralkenyl, heteroaralkynyl, or heteroaryl, heteroaralkyl, heteroaralkenyl, heteroaralkynyl substituted with one or more substituents independently selected from halo, alkyl, alkenyl, alkynyl, haloalkyl, haloalkenyl, haloalkynyl, alkoxy and haloalkoxy, heterocyclyl, heterocyclylalkyl, heterocyclylalkenyl, heterocyclylalkynyl, or heterocyclyl, heterocyclylalkyl, heterocyclylalkenyl, heterocyclylalkynyl substituted with one or more substituents independently selected from halo, alkyl, alkenyl, alkynyl, haloalkyl, haloalkeryl, haloalkynyl, alkoxy and haloalkoxy, or R³ and R⁴ taken together with the nitrogen atom to which they are attached form a 5- or 6-membered saturated or unsaturated heterocyclic ring, or
A is 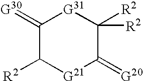
wherein each R², G²⁰, G²¹, G³⁰ and G³¹ are as previously defined,
provided that when both m and q are 0, A is 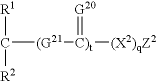
and
within the definition of R¹
d is 1,
G³⁰, G³¹, Z³, X³ and t′ are as previously defined and
Z³(X³)_{d}(G³′)ₜ is a pharmaceutical moiety wherein Z³(X³)_{d}(G³′)ₜ—H represents the pharmaceutical, or
the pharmaceutically acceptable salts, isomers, tautomers, enantiomers and mixtures thereof.

4. The pharmaceutical compound of claim 3 wherein t is 1, m is 1, q is 0, (X¹)ₘ is a nitrogen atom and Z¹(X¹)ₘ—H is a pharmaceutical, or the pharmaceutically acceptable salts, isomers, tautomers, enantiomers and mixtures thereof.

5. The pharmaceutical compound of claim 3 wherein t is 1, m is 1, q is 0, (X¹)ₘ is a phosphorous, oxygen or sulfur atom and Z¹(X¹), —H is a pharmaceutical, or t is 1, m is 0, q is 1, (X²)_{q} is a phosphorous, oxygen or sulfur atom and Z²(X²)_{q}-[(C=G²⁰)-G²¹]ₜ-H is a pharmaceutical, or the pharmaceutically acceptable salts, isomers, tautomers, enantiomers and mixtures thereof.

6. The pharmaceutical compound of claim 3 wherein t is 1, m is 0, q is 1, (2)_{q} is a carbon atom and Z²(X²)_{q}—[(C=G²⁰)-G²¹]ₜ—H is a pharmaceutical, or the pharmaceutically acceptable salts, isomers, tautomers, enantiomers and mixtures thereof.

7. The pharmaceutical compound of claim 3 wherein t is 0, m is 1, q is 0, (X¹)ₘ is a nitrogen, phosphorous, oxygen or sulfur atom and Z¹(X¹)ₘ—H is a pharmaceutical, or t is 0, m is 0, q is 1, (X²)_{q} is a nitrogen, phosphorous, oxygen, sulfur or carbon atom and Z²(X²)_{q}-[(C=G²⁰)-G²¹]ₜ—H is a pharmaceutical or
Z²(X²)_{q}—[(C=G²⁰)-G²¹]ₜ is a pharmaceutical, or the pharmaceutically acceptable salts, isomers, tautomers, enantiomers and mixtures thereof.

8. The pharmaceutical compound of claim 3 wherein t is 0 or 1, m is 1, q is 1, (X¹)ₘ is a nitrogen, phosphorous, oxygen or sulfur atom, (X²)_{q} is a nitrogen, phosphorous, oxygen, sulfur or carbon atom, Z¹(X¹)ₘ—H is a pharmaceutical and Z²(X²)_{q}—(C=G²⁰-G²′)ₜ—H or Z²(X²)_{q}—(C=G²⁰-G²¹)ₜ is a pharmaceutical, or the pharmaceutically acceptable salts, isomers, tautomers, enantiomers and mixtures thereof.

9. The pharmaceutical compound of claim 3 wherein A is 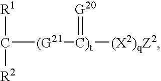
m and q are 0,
Z¹(X¹)ₘ and Z²(X²)_{q} are non-pharmaceutical moieties,
R¹ is 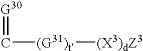
wherein
G³⁰ is an oxygen atom or a sulfur atom,
G³¹ is an oxygen atom, a sulfur atom or NR³,
d is 1,
t is 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³¹)ₜ, is a pharmaceutical moiety wherein Z³(X³)_{d}(G³′)ₜ—H represents the pharmaceutical, or
the pharmaceutically acceptable salts, isomers, tautomers, enantiomers and mixtures thereof.

10. The pharmaceutical compound of claim 3 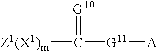
wherein 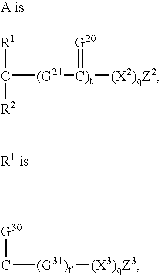
(d+m+q) is 1 or 2, or
the pharmaceutically acceptable salts, isomers, tautomers, enantiomers and mixtures thereof.

11. The pharmaceutical compound of claim 3 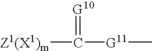
wherein 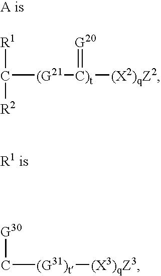
both q and t are 1,
X² is a carbon atom, or
the pharmaceutically acceptable salts, isomers, tautomers, enantiomers and mixtures thereof.

12. The pharmaceutical compound of claim 3 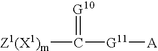
wherein
A is 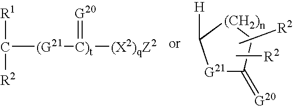
G¹⁰, G¹¹ and G²⁰ are each independently an oxygen atom or a sulfur atom,
G²¹ is an oxygen atom, a sulfur atom or NR³,
X¹ is an oxygen atom, a sulfur atom, a phosphorous atom or a nitrogen atom attached to Z¹,
X² is an oxygen atom, a sulfur atom, a phosphorous atom, a nitrogen atom or a carbon atom attached to Z²,
m, q and t are each independently 0 or 1,
n is 1 or 2,
Z¹(X¹)ₘ is a pharmaceutical moiety when m is 1 wherein Z¹(X¹)ₘ—H represents the pharmaceutical,
Z²(X²)(C(=G²′)G²1)ₜ is a pharmaceutical moiety when q is I wherein Z²(X²)_{q}(C(=G²⁰)G²¹)ₜ or Z²(X²)_{q}(C(=G²⁰)G²¹)ₜ—H represents the pharmaceutical, Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C3-CB)alkyl(C₂-C₁₀)alkenyl, cyclo(C₃-C₈)alkenyl(C₁-C₁₀)alkyl, cyclo(C3-Cs)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydrox-y, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylearbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,
Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-CB)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C3-CB)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀) alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀) alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,
R¹ is 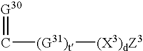
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³¹)_{t′} is a pharmaceutical moiety when d is 1 wherein Z³(X³)_{d}(G³′)ₜ—H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀) alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-Cs)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₆)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C3-CB)alkyl(C₂-C₁₀)alkynyl, cyclo(C3-Cg)alkenyl(C₁-C₁₀)alk-yl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
A is 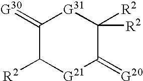
wherein each R², G²⁰, G²¹, G³⁰ and G³¹ are as previously defined,
provided that when both m and q are 0, A is 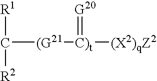
and
within the definition of R¹
d is 1,
G³⁰, G³¹, Z³, X³ and t′ are as previously defined and
Z³(X³)_{d}(G³¹)_{t′} is a pharmaceutical moiety wherein Z³(X³)_{d}(G³′)ₜ, —H represents the pharmaceutical, or
the pharmaceutically acceptable salts, isomers, tautomers, enantiomers and mixtures thereof.

13. A human health pharmaceutical compound of the formula 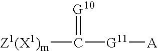
wherein
A is 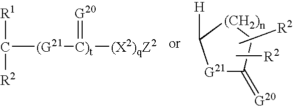
G¹⁰, G¹¹ and G²⁰ are each independently an oxygen atom or a sulfur atom,
G²¹ is an oxygen atom, a sulfur atom or NR³,
X¹ is an oxygen atom, a sulfur atom, a phosphorous atom or a nitrogen atom attached to Z¹,
X² is an oxygen atom, a sulfur atom, a phosphorous atom, a nitrogen atom or a carbon atom attached to Z²,
m is 1,
q and t are each independently 0 or 1,
n is 1 or 2,
Z¹(X¹)ₘ is a pharmaceutical moiety when m is 1 wherein Z¹(X¹)ₘ—H represents the pharmaceutical selected from the group consisting of aletamine, amoxapine, amoxicillin, amphetamine, atorvastatin, benazepril, betahistine, bupropion, carbamazepam, cefachlor, cefadroxil, centerdrine, chlordiazepoxide, chloroquine, ciprofloxacin, clonazepam, clonidine, clozapine, demethylimipramine, deprenil, desipramine, enoxacin, etintidine, fenfluramine, fludorex, fluoxetine hydrochloride, gabapentin, lansoprazole, mepivacaine, methylphenidate, molindone, naphazoline, norfloxacin, olanzapine, omeprazole, oxmetidine, paroxetine, phentermine, pimozide, piroxicam, posaconazole, prazosin, procaine, propanolol, proparacaine, quinapril, sertraline, sulfamethizole, tacrine, temazepam, terazosin, tetrahydrazoline, thiabendazole, timolol, tocainide, tolazoline, tramadol, triamterene, troglitazone and xylometazoline when X¹ is a nitrogen atom, or
the pharmaceutical selected from the group consisting of atorvastatin, atropine, bicalutamide, buprenorphine, cafiminol, clobesol, deprenil, doxazosin, enalapril, famciclovir, fluconazole, fluticasone propionate, genaconazole, haloperidol, hydroxyitraconazole, hydroxyzine, iodoquinol, loperidine hydrochloride, lorazepam, lovastatin, mazindol, metronidazole, oxycodone, quinidine, scopolamine, simvastatin, tramadol and voriconazole when X¹ is an oxygen atom, or
the pharmaceutical methimazole when X¹ is a sulfur atom,
Z²(X²)_{q}(C(=G²⁰)G²¹)ₜ is a pharmaceutical moiety when q is 1 wherein
Z²(X²)_{q}(C(=G²⁰)G²¹)ₜ or Z²(X²)_{q}(C(=G²⁰)G²′)ₜ—H represents the pharmaceutical,
Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀) alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C2-C₁₀)alkenyl, halo(C₁-C₁₀) alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C2-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR¹R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR—R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,
R¹ is 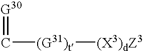
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³′)ₜ, is a pharmaceutical moiety when d is 1 wherein Z³(X³)_{d}(G³′)ₜ—H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C2-C to)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀) alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
A is 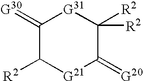
wherein each R², G²⁰, G²¹, G³⁰ and G³¹ are as previously defined,
provided that when both m and q are 0, A is 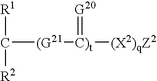
and
within the definition of R¹
d is 1,
G³⁰, G³¹, Z³, X³ and t′ are as previously defined and
Z³(X³)_{d}(G³¹)ₜ is a pharmaceutical moiety wherein Z³(X³)_{d}(G³′)_{t′}-H represents the pharmaceutical, or
the pharmaceutically acceptable salts, isomers, tautomers, enantiomers and mixtures thereof.

14. A human health pharmaceutical compound of the formula 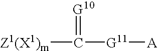
wherein
A is 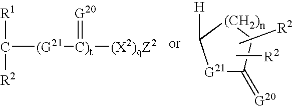
G¹⁰, G¹¹ and G²⁰ are each independently an oxygen atom or a sulfur atom,
G²¹ is an oxygen atom, a sulfur atom or NR³,
X¹ is an oxygen atom, a sulfur atom, a phosphorous atom or a nitrogen atom attached to Z¹,
X² is an oxygen atom, a sulfur atom, a phosphorous atom, a nitrogen atom or a carbon atom attached to Z²,
m and t are each independently 0 or 1,
q is 1,
n is 1 or 2,
Z¹(X¹)ₘ is a pharmaceutical moiety when m is 1 wherein Z¹(X¹)ₘ—H represents the pharmaceutical,
Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²¹)ₜ—H represents the pharmaceutical selected from the group consisting of acrivistine, aliconazole, amiodarone, amitriptyline, amoxapine, amrinone, astemizole, atropine, becliconazole, benzatropine, benzphetamine, beperiden, bisacodyl, brolaconazole, bromopheniramine, bupivacaine, caffeine, chloroprocaine, citalopram, clemastine, clomiphene, clotrimazole, colchicine, croconazole, cyclobenzaprine, cyclopentolate, cyproheptadine, democonazole, dicodid, dicyclomine, diethylproprion, diphenhydramine, diphenidol, diltiazem, diphenoxylate hydrochloride, doconazole, donezapil hydrochloride, doxapram, doxepin, eberconazole, econazole, fentanyl, fenticonazole, flavoxate, fluazepam, fluconazole, halazepam, hydroxyitraconazole, isoconazole, lansoprazole, levomethadyl, loratadine, mechlorethamine, meperidine, mepivacaine, methadone, methimazole, miconazole, minoxidil, naftifine, nefazodone, neticonazole, nifurantin, omoconazole, orconazole, orphenadrine, oxiconazole, oxybutynin, oxymetazoline, papaverine, parconazole, phenoxybenzamine, pilocarpine, pramoxine, propoxyphene, pyrazinamide, pyroxidine, ravuconazole, retinoic acid, risperidone, sertaconazole, sibutramine sufentanil, sulconazole, tamoxifen, terbinafine, ticlopidine, tioconazole, tolteridine, trihexyphenidyl, troleandomycin, tropicamide, valconazole, verapamil and zinoconazole when X² is a nitrogen atom, or
the pharmaceutical selected from the group consisting of fluconazole, genaconazole, hydroxyitraconazole, iodoquinol, lovastatin, mazindol, metronidazole, posaconazole, voriconazole and warfarin when X² is an oxygen atom, or
the pharmaceutical selected from the group consisting of adapalene, arthrocine, cloxacillin, flurbiprofen, gemfibrozil, hydroxyitraconazole, ibuprofen, indomethacin, ketoprofen, ketorolac, loratadine, monopril, oxaprozin, valproic acid and verapamil when X² is an carbon atom,
Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl; carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C3-CB)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C3-Cs)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,
R¹ is 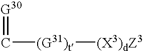
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³¹)ₜ, is a pharmaceutical moiety when d is 1 wherein Z³(X³)_{d}(G³′)ₜ—H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-CB)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₁-C₈)alkenyl(C₂-C₁₀)alkenyl, carboxycyclo(C₃-C₈)alkyl(C₂-C₁₀)alkynyl, carboxycyclo(C₁-C₁₀)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀) alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R¹ when both d and t′ are 0 and j is 0, 1 or 2,
each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀) alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C3-Cg)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀) alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
A is 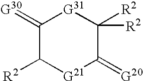
wherein each R², G²⁰, G²¹, G³⁰ and G³¹ are as previously defined,
provided that when both m and q are 0, A is 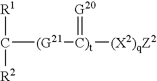
and
within the definition of R¹
d is 1,
G³⁰, G³¹, Z³, X³ and t′ are as previously defined and
Z³(X³)_{d}(G³¹)ₜ is a pharmaceutical moiety wherein Z³(X³)_{d}(G³′)ₜ—H represents the pharmaceutical, or
the pharmaceutically acceptable salts, isomers, tautomers, enantiomers and mixtures thereof.

15. A veterinary pharmaceutical compound of the formula 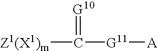
wherein
A is 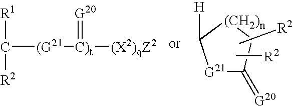
G¹⁰, G¹¹ and G²⁰ are each independently an oxygen atom or a sulfur atom,
G²¹ is an oxygen atom, a sulfur atom or NR³,
X¹ is an oxygen atom, a sulfur atom, a phosphorous atom or a nitrogen atom attached to Z¹,
X² is an oxygen atom, a sulfur atom, a phosphorous atom, a nitrogen atom or a carbon atom attached to Z²,
m is 1,
q and t are each independently 0 or 1,
n is 1 or 2,
Z¹(X¹)ₘ is a pharmaceutical moiety when m is 1 wherein Z¹(X¹)ₘ—H represents the pharmaceutical selected from the group consisting of albendazole, aminocaproic acid, aminophylline, amprolium, atipamezole, benazepril, cisapride, detomridine, disopyramide, enalapril, febantel, fluconazole, imidacloprid, ketamine, lidocaine, lincomycin, lomustine, mechlorethamine, meclofenamic acid, mercaptopurine, methotrexate, mexiletine, ormetoprim, piroxicam, primidone, procainamide, prostaglandin E1, quinacrine, quinidine, sulfachlorpyridazine, sulfadiazine, sulfamethoxazole, theophylline, thiabendazole, tiletamine, tocainide, vincristine and xylazine when X¹ is a nitrogen atom, or
the pharmaceutical selected from the group consisting of clioquinol, enalapril, guaifenesin, mibolerone, oxazepam, prostaglandin E1 and warfarin when X¹ is an oxygen atom,
Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²′)ₜ-H represents the pharmaceutical,
Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀) alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C3-CB)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,
R¹ is 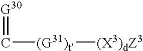
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³′)ₜ, is a pharmaceutical moiety when d is 1 wherein Z³(X³)_{d}(G³¹)ₜ—H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C4)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C2-C₁₋₆)alkynyl, halo(C₁-C₁₀)alkyl, halo(C₂-C₁₀)alkenyl, halo(C₂-C₁₀)alklynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
A is 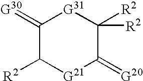
wherein each R², G²⁰, G²¹, G³⁰ and G³′ are as previously defined,
provided that when both m and q are 0, A is 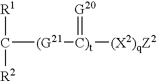
and
within the definition of R¹
d is 1,
G³⁰, G³¹, Z³, X³ and t′ are as previously defined and
Z³(X³)_{d}(G³¹)_{t′} is a pharmaceutical moiety wherein Z³(X³)_{d}(G³′)_{t′}-H represents the pharmaceutical, or
the pharmaceutically acceptable salts, isomers, tautomers, enantiomers and mixtures thereof.

16. A veterinary pharmaceutical compound of the formula 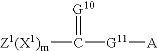
wherein
A is 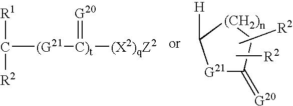
G¹⁰, G¹¹ and G²⁰ are each independently an oxygen atom or a sulfur atom,
G²¹ is an oxygen atom, a sulfur atom or NR³,
X¹ is an oxygen atom, a sulfur atom, a phosphorous atom or a nitrogen atom attached to Z¹,
X² is an oxygen atom, a sulfur atom, a phosphorous atom, a nitrogen atom or a carbon atom attached to Z²,
m and t are each independently 0 or 1,
q is 1,
n is 1 or 2,
Z¹(X¹)ₘ is a pharmaceutical moiety when m is 1 wherein Z¹(X¹)ₘ—H represents the pharmaceutical,
Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²′)ₜ—H represents the pharmaceutical selected from the group consisting of acepromazine, albendazole, aminopropazine, amiodarone, amitriptyline, atropine, azaperone, buspirone, captopril, cephapirin, chlorpheniramine, clemastine, clomipramine, cyproheptadine, diethylcarbamazine, diltiazem, diphenhydramine, diphenoxylate, doxapram, doxepin, doxylamine, droperidol, febantel, fenbendazole, fentanyl, hetacillin, hydrocodone, hydroxyzine, itraconazole, levamisole, meclizine, meperidine, methenamine, morantel, naltrexone, omeprazole, oxybutynin, pentazocine, piroxicam, primidone, procainamide, promazine, pyrantel, selegiline, thiabendazole, tiamulin and verapamil when X² is a nitrogen atom, or
the pharmaceutical selected from the group consisting of clioquinol, guaifenesin, mibolerone, oxazepam and warfarin when X² is an oxygen atom, or
the pharmaceutical selected from the group consisting of ketoprofen and valproic acid when X² is a carbon atom,
Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C3-Cs)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀) alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,
R¹ is 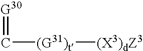
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³1)_{t′} is a pharmaceutical moiety when d is 1 wherein Z³(X³)_{d}(G³′)ₜ—H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanatq, (C₁-C₁₀)alkyl, (C₁-C₁₀)alkylstlfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(Cs-C8)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
each R² is independently a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR¹R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
A is 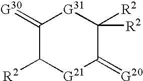
wherein each R², G²⁰, G²¹, G³⁰ and G³¹ are as previously defined,
provided that when both m and q are 0, A is 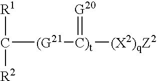
and
within the definition of R¹
d is 1,
G³⁰, G³¹, Z³, X³ and t′ are as previously defined and
Z³(X³)_{d}(G³¹)_{t′} is a pharmaceutical moiety wherein Z³(X³)_{d}(G³′)_{t′}-H represents the pharmaceutical, or
the pharmaceutically acceptable salts, isomers, tautomers, enantiomers and mixtures thereof.

17. A pharmaceutical compound of the formula 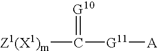
wherein
A is 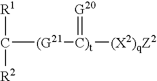
G¹⁰, G¹¹ and G²⁰ are each independently an oxygen atom or a sulfur atom,
G²¹ is an oxygen atom, a sulfur atom or NR³,
X¹ is a nitrogen atom attached to Z¹,
X² is an oxygen atom, a sulfur atom, a phosphorous atom, a nitrogen atom or a carbon atom attached to Z²,
m is 1,
q and t are each independently 0 or 1,
Z¹(X¹)ₘ is a pharmaceutical moiety selected from 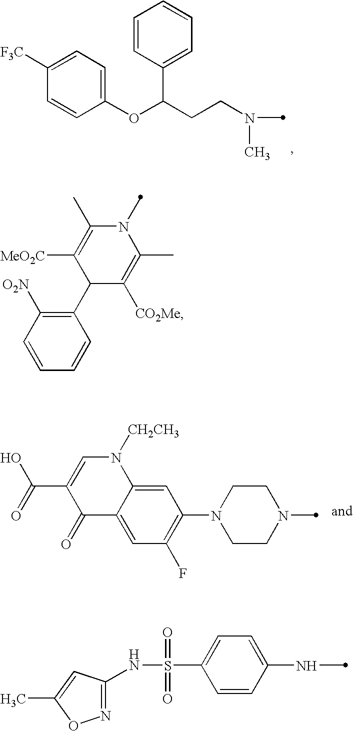
wherein Z¹(X¹)ₘ—H represents the respective pharmaceutical selected from 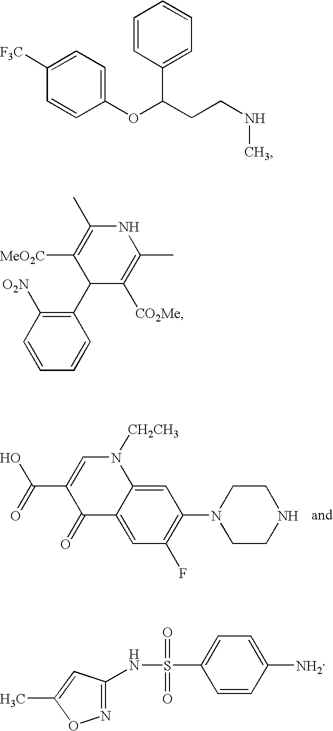
-● represents the connection point between said pharmaceutical moiety and the moiety represented by 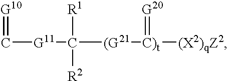
Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²′)ₜ—H represents the pharmaceutical,
Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₈)alkoxy(C₁-C₈)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀) alkenyl, halo(C₁-C₁₀)alkylthio(C₂-C₁₀)alkynyl, SO₂NR³R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,
Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,
G²¹ is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M⁻}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,
R¹ is 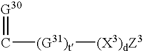
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³¹)ₜ, is a pharmaceutical moiety when d is 1 wherein Z³(X³)_{d}(G³′)_{t′}-H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-Cs)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R⁴ when both d and t′ are 0 and j is 0, 1 or 2,
(d+q)is 0 or 1,
R² is a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C₄)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
the pharmaceutically acceptable salts, isomers, tautomers, enantiomers and mixtures thereof.

18. A pharmaceutical compound of the formula 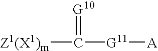
wherein
A is 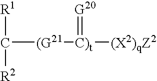
G¹⁰, G¹¹ and G²⁰ are each independently an oxygen atom or a sulfur atom,
G²¹ is an oxygen atom, a sulfur atom or NR³,
X¹ is an oxygen atom attached to Z¹,
X² is an oxygen atom, a sulfur atom, a phosphorous atom, a nitrogen atom or a carbon atom attached to Z²,
m is 1,
q and t are each independently 0 or 1,
Z¹(X¹)ₘ is a pharmaceutical moiety selected from 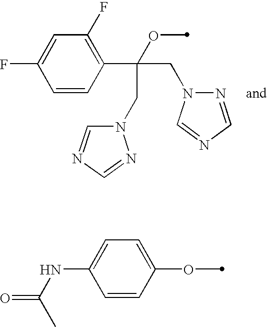
wherein Z¹(X¹), —H represents the respective pharmaceutical selected from 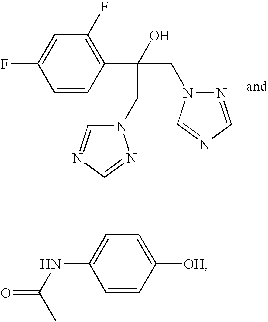
-● represents the connection point between said pharmaceutical moiety and the moiety represented by 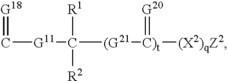
Z²(X²)_{q}(C(=G²⁰)G²¹)ₜ is a pharmaceutical moiety when q is 1 wherein Z²(X²)_{q}(C(=G²⁰)G²¹)ₜ or Z²(X²)_{q}(C(=G²⁰)G²′)ₜ-H represents the pharmaceutical,
Z¹(X¹)ₘ, when m is 0, is a hydrogen atom, halo, (C₁-C₂₀)alkyl, (C₁-C₁₀)alkylcarbonyloxy(C₁-C₁₀)alkyl, (C₁-C₂₀)alkylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR¹R⁴, NR³R⁴, OR³, S(O)ⱼR³, carboxy(C₁-C₂₀)alkyl, carboxy(C₂-C₂₀)alkenyl, carboxy(C₂-C₂₀)alkynyl, aryl, arylcarbonyl, arylcarbonyl(C₁-C₁₀)alkyl, aroxycarbonyl, aroxycarbonyl(C₁-C₁₀)alkyl, or aryl, arylcarbonyl, arylcarbonyl(C₁-C₁₀)alkyl, aroxycarbonyl, aroxycarbonyl(C₁-C₁₀)alkyl substituted with one or more substituents independently selected from halo, nitro, cyano, hydroxy, (C₁-C₁₀) alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴, and NR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, ar(C₁-C₁₀)alkylcarbonyl, ar(C₁-C₁₀)alkylcarbonyl(C₁-C₁₀)alkyl, ar(C₂-C₁₀)alkenylcarbonyl, ar(C₂-C₁₀)alkenylcarbonyl(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyl, heteroarylcarbonyl(C₁-C₁₀)alkyl, heteroaroxycarbonyl, heteroaroxycarbonyl(C₁-C₁₀)alkyl, or heteroaryl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl, or heteroar(C₁-C₁₀)alkyl, heteroar(C₂-C₁₀)alkenyl, heteroar(C₂-C₁₀)alkynyl, heteroar(C₁-C₁₀)alkylcarbonyl, heteroar(C₁-C₁₀)alkylcarbonyl(C₁-C₁₀)alkyl, heteroar(C₂-C₁₀)alkenylcarbonyl, heteroar(C₂-C₁₀)alkenylcarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴ wherein j is 0, 1 or 2,
Z²(X²)_{q} is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₂₀)alkenylcarbonyl, (C₁-C₂₀)alkynylcarbonyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, halo(C₁-C₂₀)alkyl, (C₂-C₂₀)alkenyl, halo(C₂-C₂₀)alkenyl, (C₁-C₁₀)alkylcarbonylamino(C₂-C₁₀)alkenyl, arylcarbonylamino(C₂-C₁₀)alkenyl, heteroarylcarbonylamino(C₂-C₁₀)alkenyl, (C₂-C₂₀)alkynyl, halo(C₂-C₂₀)alkynyl, cyclo(C₃-C₈)alkyl, cyclo(C3-CB)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C3-Cg)alkenyl(C₂-C₁₀)alkynyl, carboxycyclo(C₃-C₈)alkyl(C₁-C₁₀)alkyl, carboxy(C₃-C₈)cycloalkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C3-CB)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₅)alkoxy(C₁-C₅)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, SO₂NR³R⁴, NR³R⁴, carboxy(C₁-C₂₀)alkyl, carboxy(C₂-C₂₀)alkenyl, carboxy(C₂-C₂₀)alkynyl, di(C₁-C₁₀)alkoxyphosphoryl(C₁-C₁₀)alkyl, aryl, aryl substituted with one or more substituents independently selected from halo, nitro, cyano, hydroxy, (C₁-C₁₀)alkyl, (C₁-C₁₀)alkylsulfonyl(C₁-C₁₀)alkyl, (C₁-C₁₀)alkylsulfonyl, thiocyanato, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, C(═O)OR², C(═O)SR², C(═S)OR², C(═S)SR², C(═O)NR³R⁴, C(═S)NR³R⁴, C(═O)R², C(═S)R², C(═N—R³)R², C(═N—OR³)R², C(═N—NR³R⁴)R², OP(═O)(OR²)₂, SO₂NR³R⁴, NR³R⁴ and (C₁-C₁₀)alkylNR³R⁴, ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alknyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, (C₁-C₁₀)alkylcarbonyl(C₁-C₁₀)alkyl, (C₂-C₁₀)alkenylcarbonyl(C₁-C₁₀)alkyl, (C₂-C₁₀)alkynylcarbonyl(C₁-C₁₀)alkyl, heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl, or heterocyclyl, heterocyclyl(C₁-C₁₀)alkyl, heterocyclyl(C₂-C₁₀)alkenyl, heterocyclyl(C₂-C₁₀)alkynyl, heterocyclylcarbonyl, heterocyclylcarbonyl(C₁-C₁₀)alkyl, heterocyclyloxycarbonyl, heterocyclyloxycarbonyl(C₁-C₁₀)alkyl, arylcarbonyl, arylcarbonyl(C₁-C₁₀)alkyl, ar(C₁-C₁₀)alkylcarbonyl, ar(C₁-C₁₀)alkylcarbonyl(C₁-C₁₀)alkyl, aroxycarbonyl, aroxycarbonyl(C₁-C₁₀)alkyl, ar(C₁-C₁₀)alkoxycarbonyl, ar(C₁-C₁₀)alkoxycarbonyl(C₁-C₁₀)alkyl, heteroarylcarbonyl, heteroarylcarbonyl(C₁-C₁₀)alkyl, heteroaroxycarbonyl, heteroaroxycarbonyl(C₁-C₁₀)alkyl substituted with one or more substituents independently selected from halo, hydroxy, nitro, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, and C(═N-G²²)R² when q is 0 and t is 1,
G²² is OR³, OCOR³, S(O)ⱼR³, OS(O)ⱼR³, NR³R⁴, OSO₂NR³R⁴, OP(═O)OR³NR³R⁴, OP(═O)(OR³)₂ or N═CR³R⁴,
j is 0, 1 or 2,
Z²(X²)_{q} is halo, NR³R⁴, {(NR³R⁴R⁵)+M-}, OR³, S(O)ⱼR³ or SO₂NR³R⁴ when both q and t are 0 wherein M⁻ is halo, hydroxy, (C₁-C₈)alkoxy or the anion of a carboxylic acid and j is 0, 1 or 2,
R¹ is 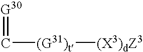
wherein
G³⁰ is an oxygen atom or a sulfur atom,
G³¹ is an oxygen atom, a sulfur atom or NR³,
t′ and d are each independently 0 or 1,
X³ is an oxygen atom, a sulfur atom, a nitrogen atom, a phosphorous atom or a carbon atom attached to Z³ when t′ is 0, a nitrogen atom attached to Z³ when t′ is 1 and G³¹ is NR³, or a carbon atom attached to Z³ when t′ is 1 and G³¹ is an oxygen atom or a sulfur atom,
Z³(X³)_{d}(G³¹)_{t′} is a pharmaceutical moiety when d is 1 wherein Z³(X³)_{d}(G³¹)_{t′}-H represents the pharmaceutical,
Z³(X³)_{d}, when d is 0 and t′ is 1, is a hydrogen atom, (C₁-C₂₀)alkyl, (C₁-C₁₀)alkylcarbonyloxy(C₁-C₁₀)alkyl, (C₁-C₂₀)alkylcarbonyl, (C₁-C₁₀)alkylcarbonyl(C₁-C₁₀)alkyl, hydroxy(C₁-C₂₀)alkyl, (C₁-C₁₀)alkylsulfonyl(C₁-C₁₀)alkyl, (C₁-C₁₀)alkylcarbonylamino(C₁-C₁₀)alkyl, arylcarbonylamino(C₁-C₁₀)alkyl, heteroarylcarbonylamino(C₁-C₁₀)alkyl, acetylamino(C₁-C₁₀)alkyl, halo(C₁-C₂₀)alkyl, (C₂-C₁₀)alkenyl, (C₂-C₁₀)alkenylcarbonyl(C₁-C₁₀)alkyl, acetylamino(C₂-C₁₀)alkenyl, halo(C₂-C₁₀)alkenyl, (C₂-C₁₀)alkynyl, (C₂-C₁₀)alkynylcarbonyl(C₁-C₁₀)alkyl, halo(C₂-C₁₀)alkynyl, cyclo(C₃-C₈)alkyl, cyclo(C₃-C₈)alkenyl, carboxycyclo(C₃-C₈)alkyl, carboxycyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkenyl(C₁-C₁₀)alkyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, carboxycyclo(C₃-C₈)alkyl(C₁-C₁₀)alkyl, carboxycyclo(C₃-C₈)alkyl(C₂-C₁₀)alkenyl, carboxycyclo(C₃-C₈)alkenyl(C₁-C₁₀)alkyl, carboxycyclo(C₃-C₈)alkenyl(C₂-C₁₀)alkenyl, carboxycyclo(C₃-C₈)alkyl(C₂-C₁₀)alkynyl, carboxycyclo(C₃-C₈)alkenyl(C₂-C₁₀)alkynyl, (C₁-C₁₀)alkoxy(C₁-C₁₀)alkyl, (C₁-C₁₀)alkoxy(C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, halo(C₁-C₁₀)alkoxy(C₁-C₁₀)alkyl, halo(C₁-C₁₀)alkoxy(C₂-C₁₀)alkenyl, halo(C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, halo(C₁-C₁₀)alkylthio(C₁-C₁₀)alkyl, halo(C₁-C₁₀)alkylthio(C₂-C₁₀)alkenyl, halo(C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, NR³R⁴, OR³, S(O)ⱼR³, aryl, arylcarbonyloxy(C₁-C₁₀)alkyl, arylcarbonyl(C₁-C₁₀)alkyl, aroxycarbonyl(C₁-C₁₀)alkyl, or aryl, arylcarbonyloxy(C₁-C₁₀)alkyl, arylcarbonyl(C₁-C₁₀)alkyl, aroxycarbonyl(C₁-C₁₀)alkyl substituted with one or more substituents independently selected from halo, nitro, hydroxy, cyano, thiocyanato, (C₁-C₁₀)alkyl, (C₁-C₁₀)alkylsulfonyl(C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl, or ar(C₁-C₁₀)alkyl, ar(C₁-C₁₀)alkylcarbonyloxy(C₁-C₁₀)alkyl, ar(C₁-C₁₀)alkylcarbonyl(C₁-C₁₀)alkyl, ar(C₁-C₁₀)alkoxycarbonyl(C₁-C₁₀)alkyl, ar(C₂-C₁₀)alkenyl, ar(C₂-C₁₀)alkynyl, arcyclo(C₃-C₈)alkyl, aroxy(C₁-C₁₀)alkyl substituted with one or more substituents independently selected from halo, nitro, hydroxy, cyano, (C₁-C₁₀)alkyl, cyclo(C₃-C₈)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl, or heteroaryl, heteroarylcarbonyloxy(C₁-C₁₀)alkyl, heteroarylcarbonyl(C₁-C₁₀)alkyl, heteroaroxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroar(C₁-C₁₀)alkyl, heteroar(C₂-C₁₀)alkenyl, heteroar(C₂-C₁₀)alkynyl, or heteroar(C₁-C₁₀)alkyl, heteroar(C₂-C₁₀)alkenyl, heteroar(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heterocyclyl, heterocyclylcarbonyloxy(C₁-C₁₀)alkyl, hetercyclylcarbonyl(C₁-C₁₀)alkyl, heterocyclyloxycarbonyl(C₁-C₁₀)alkyl, or heterocyclyl, heterocyclylcarbonyloxy(C₁-C₁₀)alkyl, heterocyclylcarbonyl(C₁-C₁₀)alkyl, heterocyclyloxycarbonyl(C₁-C₁₀)alkyl substituted with one or more substituents independently selected from halo, nitro, hydroxy, cyano, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, wherein j is 0, 1 or 2,
Z³(X³)_{d} is halo, NR³R⁴, OR³, N(R³)—N═CR³R⁴, S(O)ⱼR³ or SO₂NR³R¹ when both d and t′ are 0 and is 0, 1 or 2,
(d+q)is 0 or 1,
R² is a hydrogen atom, (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C2-C₁₀)alkenyl, (C₁-C₁₀)alkylthio(C₂-C₁₀)alkynyl, carboxy, a carboxylate salt, carboxy(C₁-C₂₀)alkyl, carboxy(C₂-C₂₀)alkenyl, carboxy(C₂-C₂₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₂₀)alkenylcarbonyl, (C₂-C₂₀)alkynylcarbonyl, Cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl, or (C₁-C₂₀)alkyl, (C₂-C₁₀)alkenyl, (C₂-C₁₀)alkynyl, (C₁-C₁₀)alkoxy(C₁-C₁₀)alkyl, (C₁-C₁₀)alkoxy(C₂-C₁₀)alkenyl, (C₁-C₁₀)alkoxy(C₂-C₁₀)alkynyl, (C₁-C₁₀)alkylthio(C₁-C₁₀)alkyl, (C₁-C₁₀)alkylthio(C₂-C₁₀)alkenyl, (C₁-C₁₀)alkylthio(C₂-C₁₀)alkynyl, carboxy(C₁-C₂₀)alkyl, carboxy(C₂-C₁₀)alkenyl, carboxy(C₂-C₁₀)alkynyl, (C₁-C₂₀)alkoxycarbonyl, (C₁-C₁₀)alkoxycarbonyl(C₁-C₁₀)alkyl, (C₁-C₁₀)alkoxycarbonyl(C₂-C₁₀)alkenyl, (C₁-C₁₀)alkoxycarbonyl(C₂-C₁₀)alkynyl, (C₁-C₂₀)alkylcarbonyl, (C₂-C₁₀)alkenylcarbonyl, (C₂-C₁₀)alkynylcarbonyl, cyclo(C₃-C₈)alkyl, cyclo(C₃-C₈)alkenyl, cyclo(C₃-C₈)alkyl(C₁-C₁₀)alkyl, cyclo(C₃-C₈)alkenyl(C₁-C₁₀)alkyl, cyclo(C₃-C₈)alkyl(C₂-C₁₀)alkenyl, cyclo(C₃-C₈)alkenyl(C₂-C₁₀)alkenyl, cyclo(C₃-C₈)alkyl(C₂-C₁₀)alkynyl, cyclo(C₃-C₈)alkenyl(C₂-C₁₀)alkynyl, heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, cyano, hydroxy, nitro, SO₂NR³R⁴ and NR³R⁴, aryl or aryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, carboxy, (C₁-C4)alkoxycarbonyl, SO₂NR³R⁴ and NR³R⁴, ar(C₁-C₁₀)alkyl, ar(C₂-C₁₀)alkenyl, ar(C₂-C₁₀)alkynyl, or ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl, or arylcarbonyl, ar(C₁-C₁₀)alkylcarbonyl, ar(C₂-C₁₀)alkenylcarbonyl, ar(C₂-C₁₀)alkynylcarbonyl, aroxycarbonyl(C₁-C₁₀)alkyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroaryl, heteroaryl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy and NR³R⁴, heteroar(C₁-C₁₀)alkyl, heteroar(C₂-C₁₀)alkenyl, heteroar(C₂-C₁₀)alkynyl or heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl, or heteroarylcarbonyl, heteroar(C₁-C₁₀)alkylcarbonyl, heteroar(C₂-C₁₀)alkenylcarbonyl, heteroar(C₂-C₁₀)alkynylcarbonyl, heteroaroxycarbonyl(C₁-C₁₀)alkyl, heterocyclylcarbonyl, heterocyclyloxycarbonyl(C₁-C₁₀)alkyl substituted with one or more substituents independently selected from halo, cyano, hydroxy, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R¹ and R² taken together with the carbon atom to which they are attached form a 5-7 membered saturated or unsaturated ring,
R³, R⁴ and R⁵ are each independently a hydrogen atom, (C₁-C₂₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl, (C₂-C₁₀)alkenyl, (C₂-C₁₀)alkynyl, or (C₁-C₁₀)alkyl, cyclo(C₃-C₈)alkyl, cyclo(C₃-C₈)alkenyl, cyclo(C₃-C₈)alkyl(C₁-C₁₀)alkyl, cyclo(C₃-C₈)alkyl(C₂-C₁₀)alkenyl, cyclo(C₃-C₈)alkyl(C₂-C₁₀)alkynyl, cyclo(C₃-C₈)alkenyl(C₁-C₁₀)alkyl, cyclo(C₃-C₈)alkenyl(C₂-C₁₀)alkenyl, cyclo(C₃-C₈)alkenyl(C₂-C₁₀)alkynyl, carboxy(C₁-C₂₀)alkyl, carboxy(C₂-C₁₀)alkenyl, carboxy(C₂-C₁₀)alkynyl, (C₁-C₁₀)alkoxy(C₁-C₁₀)alkyl; (C₂-C₁₀)alkenyl or (C₂-C₁₀)alkynyl substituted with one or more halo, aryl, ar(C₁-C₁₀)alkyl, ar(C₂-C₁₀)alkenyl, ar(C₂-C₁₀)alkynyl or aryl, ar(C₁-C₁₀)alkyl, ar(C₂-C₁₀)alkenyl, ar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heteroaryl, heteroar(C₁-C₁₀)alkyl, heteroar(C₂-C₁₀)alkenyl, heteroar(C₂-C₁₀)alkynyl or heteroaryl, heteroar(C₁-C₁₀)alkyl, heteroar(C₂-C₁₀)alkenyl, heteroar(C₂-C₁₀)alkynyl substituted with one or more substituents independently selected from halo, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy and halo(C₁-C₁₀)alkoxy, heterocyclyl, heterocyclyl(C₁-C₁₀)alkyl, heterocyclyl(C₂-C₁₀)alkenyl, heterocyclyl(C₂-C₁₀)alkynyl, or heterocyclyl, heterocyclyl(C₁-C₁₀)alkyl, heterocyclyl(C₂-C₁₀)alkenyl, heterocyclyl(C₂-C₁₀)alkynyl substituted with one or more substituents independently selected from halo, hydroxy, cyano, nitro, (C₁-C₁₀)alkyl, (C₂-C₁₀)alkenyl, (C₂-C₁₀)alkynyl, halo(C₁-C₁₀)alkyl, halo(C₂-C₁₀)alkenyl, halo(C₂-C₁₀)alkynyl, (C₁-C₁₀)alkoxy, halo(C₁-C₁₀)alkoxy, SO₂NR³R⁴ and NR³R⁴, or R³ and R⁴ taken together with the nitrogen atom to which they are attached form a 5- or 6-membered saturated or unsaturated heterocyclic ring, or
the pharmaceutically acceptable salts, isomers, tautomers, enantiomers and mixtures thereof.

19. The pharmaceutical composition of any one of claims 3-18 comprising a pharmaceutical compound of this invention and a pharmaceutically acceptable carrier.

20. The composition of claim 19 containing from about 0.1% to about 99% by weight of said pharmaceutical compound.

21. A method of controlling pain or disease symptoms in a warm-blooded animal exhibiting pain or disease symptoms comprising administering thereto a pharmaceutically effective amount of a compound of any one of claims 3-18.

22. A method of controlling pain or disease symptoms in a warm-blooded animal exhibiting pain or disease symptoms comprising administering thereto a pharmaceutically effective amount of the composition of claim 19.

23. A method of controlling pain or disease symptoms in a warm-blooded animal exhibiting pain or disease symptoms comprising administering thereto a pharmaceutically effective amount of the composition of claim 20.
